(19) United States
(12) Patent Application Publication
(10) Pub. No.: US 2020/0208114 A1
(43) Pub. Date: Jul. 2, 2020

(54) TAXONOMY AND USE OF BONE MARROW STROMAL CELL

(71) Applicants: The Broad Institute, Inc., Cambridge, MA (US); Massachusetts Institute of Technology, Cambridge, MA (US); The General Hospital Corporation, Boston, MA (US); President and Fellows of Harvard College, Cambridge, MA (US)

(72) Inventors: Ninib Baryawno, Boston, MA (US); Dariusz Przybylski, Cambridge, MA (US); Monika Kowalczyk, Cambridge, MA (US); Aviv Regev, Cambridge, MA (US); David T. Scadden, Boston, MA (US)

(21) Appl. No.: 16/709,710

(22) Filed: Dec. 10, 2019

Related U.S. Application Data
(60) Provisional application No. 62/777,606, filed on Dec. 10, 2018.
(60) Provisional application No. 62/808,177, filed on Feb. 20, 2019.

(51) Int. Cl. C12N 5/0775; G01N 33/50; C12N 15/11; C12N 9/22; A61K 35/28; C12Q 1/6886

(52) U.S. Cl. CPC C12N 5/0663; G01N 33/5044; C12N 15/11; C12N 9/22; A61K 35/28; C12Q 2600/136; C12N 2800/80; C12N 2310/20; C12N 2510/00; C12N 2503/02; C12Q 2600/158; C12Q 1/6886

(57) ABSTRACT

Described herein are signatures that characterize a particular stromal cell state, type, and/or subtype. In some embodiments, the signatures can characterize a dysfunctional stromal cell. In some embodiments, the signatures can be used to diagnose, treat, and/or prevent a disease. In some embodiments, the signatures can characterize remodeling in a bone marrow microenvironment. Also described herein are cell populations having a specific signature and modulated cells that can be modulate to have a specific signature.

### CROSS-REFERENCE TO RELATED APPLICATIONS

This application claims the benefit of U.S. Provisional Application No. 62/777,606 filed Dec. 10, 2018. This application claims the benefit of U.S. Provisional Application No. 62/808,177, filed Feb. 20, 2019. The entire contents of the above-identified applications are hereby fully incorporated herein by reference.

### STATEMENT REGARDING FEDERALLY SPONSORED RESEARCH

This invention was made with government support under Grant No. DK107784 granted by National Institutes of Health. The government has certain rights in the invention.

### SEQUENCE LISTING

This application contains a sequence listing filed in electronic form as an ASCII.txt file entitled BROD-4510US_ST25.txt, created on Dec. 4, 2019 and having a file size of 13 KB. The content of the sequence listing is incorporated herein in its entirety.

### TECHNICAL FIELD

The subject matter disclosed herein is generally directed to bone marrow stromal cell populations, gene signatures and profiles of bone marrow stromal cells, characterizing and modulating aspects of bone marrow stromal cell(s), identification of distinct normal and dysfunctional bone marrow stromal cell populations, types, subtypes, gene signatures and profiles, and identification of modifications to bone marrow microenvironment in both health and disease. The subject matter disclosed herein is generally directed to modulation of bone marrow stromal cells to treat disease.

### BACKGROUND

The tissue microenvironment of stem cell niches maintains and regulates stem cell function through cellular interactions and secreted factors (Scadden, 2014; Schofield, 1978). Hematopoiesis provides a paradigm for understanding mammalian stem cells and their niches, with pivotal understanding from numerous in vivo studies on the critical role of several non-hematopoietic niche cells as regulators of hematopoietic stem cell (HSC) function (Calvi et al., 2003; Ding et al., 2012; Kunisaki et al., 2013; Mendez-Ferrer et al., 2010; Zhang et al., 2003).

One major component are multipotent mesenchymal stem/stromal cells (MSCs), non-hematopoietic cells derived from the mesoderm with potential to differentiate into bone, fat and cartilage in vitro (Kfoury and Scadden, 2015). While MSCs are found in most tissues, their diversity and lineage relationships are incompletely understood. For instance, several subtypes of MSCs have been described in specialized niches that regulate HSC maintenance. Most of these cells are located in the perivascular space and associated with either arteriole or sinusoidal blood vessels, produce key niche factors such as Cxcl12 and Stem Cell Factor (SCF, also known as Kitl) (Morrison and Scadden, 2014), and are identified by Leptin receptor [Lepr-cre] (Ding and Morrison, 2013; Ding et al., 2012), Nestin [Nes-GFP] (Mendez-Ferrer et al., 2010) or Ng2 (Cspg4) [NG2-CreER] (Kunisaki et al., 2013) expression. However, it remains unclear if these markers delineate distinct or overlapping cell populations.

Other non-hematopoietic cells, including endothelial cells (ECs) and MSC-descendent osteolineage cells (OLCs), also play roles as niche cells. Endothelial cells produce Cxcl12, SCF, and other niche factors and are critical regulators of HSC function (Butler et al., 2010; Ding et al., 2012; Doan et al., 2013; Hooper et al., 2009; Itkin et al., 2016; Kobayashi et al., 2010; Kusumbe et al., 2016). OLCs are critical for HSC homing after lethal irradiation and bone marrow transplantation (Lo Celso et al., 2009), modulate hematopoietic progenitor function and lineage maturation (Ding and Morrison, 2013; Yu et al., 2016; Yu et al., 2015), and dysfunction in some of them has been implicated in myelodysplasia and leukemia development (Dong et al., 2016; Kode et al., 2014; Raaijmakers et al., 2010; Zambetti et al., 2016).

However, despite extensive studies, the HSC niche remains incompletely defined in terms of its cellular and molecular composition, limiting our ability to prospectively isolate and functionally characterize niche cells. Previous profiling studies of MSCs were performed in bulk and relied on reporter genes to purify cell populations (Morrison and Scadden, 2014), which may either analyze a mixed population (if marker expression is more promiscuous than assumed), only cover a subset (if the marker is overly specific), or fail to detect unknown or transient states.

Citation or identification of any document in this application is not an admission that such a document is available as prior art to the present invention.

### SUMMARY

In some exemplary embodiments, described herein are methods of remodeling a stromal cell landscape comprising administering a modulating agent to a subject or a cell population that induces a shift in the stromal cell landscape from a disease-associated stromal cell landscape to a homeostatic stromal cell landscape.

In some exemplary embodiments, the shift in stromal cells from a disease-associated stromal cell landscape to a homeostatic stromal cell landscape comprises a change in the proportion of preosteoblasts. In some exemplary embodiments, the change in the proportion of preosteoblasts comprises a change in the relative proportion of OLC-1 cells to OLC-2 cells. In some exemplary embodiments, the change in the relative proportion of OLC-1 cells to OLC-2 cells comprises a decrease in OLC-1 cells and an increase in OLC-2 cells.

In some exemplary embodiments, the shift in stromal cells from a disease-associated stromal cell landscape to a homeostatic stromal cell landscape comprises a change in the relative proportion of bone marrow derived endothelial cell subtypes. In some exemplary embodiments, the change in the relative proportion of bone marrow derived endothelial cell subtypes comprises an increase in sinusoidal bone marrow derived endothelial cells and a decrease in arterial bone marrow derived endothelial cells.

In some exemplary embodiments, the shift in stromal cells from a disease-associated stromal cell landscape to a homeostatic stromal cell landscape comprises a change in the relative proportion of chondrocyte subtypes. In some exemplary embodiments, the change in the relative proportion of chondrocyte subtypes comprises a decrease in chondrocyte hypertrophic cell subtype and an increase in chondrocyte progenitor cell subtype.

In some exemplary embodiments, the shift in stromal cells from a disease-associated stromal cell landscape to a homeostatic stromal cell landscape comprises a change in the relative proportion of fibroblast subtypes. In some exemplary embodiments, the change in the relative proportion of fibroblast subtypes comprises an increase in fibroblast subtype-3 and a decrease in fibroblast subtype-4.

In some exemplary embodiments, the shift in stromal cells from a disease-associated stromal cell landscape to a homeostatic stromal cell landscape comprises a change in the relative proportion in mesenchymal stem/stromal cell (MSC) subtypes. In some exemplary embodiments, the change in the relative proportion in mesenchymal stem/stromal cell (MSC) sub-types comprises a decrease in MSC-2 subtype and an increase in MSC-3 and MSC-4 subtypes.

In some exemplary embodiments, the shift in the stromal cell landscape comprises a change in the distance in gene expression space between OLC-1, OLC-2, bone marrow derived endothelial cell subtypes, chondrocyte subtypes, fibroblast subtypes, mesenchymal stem/stromal cell (MSC) subtypes, or a combination thereof. In some exemplary embodiments, the distance is measured by a Euclidean distance, Pearson coefficient, Spearman coefficient, or a combination thereof. In some exemplary embodiments, the gene expression space comprises 10 or more genes, 20 or more genes, 30 or more genes, 40 or more genes, 50 or more genes, 100 or more genes, 500 or more genes, or 1000 or more genes. In some exemplary embodiments, remodeling the stromal cell landscape comprises increasing or decreasing the expression of one or more genes, gene programs, gene expression cassettes, gene expression signatures, or a combination thereof. In some exemplary embodiments, the change in the gene expression space is characterized by a change in the expression of one or more genes as in any of Tables 1-8 or an expression signature derived therefrom. In some exemplary embodiments, identifying differences in stromal cell states in the shift in the stromal cell landscape comprises comparing a gene expression distribution of a stromal cell type or subtype in the diseased stromal cell landscape with a gene expression distribution of the stromal cell type or subtype in the homeostatic stromal cell landscape as determined by single cell RNA-sequencing (scRNA-seq).

In some exemplary embodiments, the shift in the stromal cell landscape from a disease-associated stromal cell landscape to a homeostatic stromal cell landscape increases committed MSCs and decreases osteoprogenitor cells.

In some exemplary embodiments, the subject suffers from a hematological disease. In some exemplary embodiments, the hematological disease is a blood cancer. In some embodiments, the blood cancer is leukemia. In some embodiments, the blood cancer is acute lymphocytic leukemia, acute myeloid leukemia, chronic lymphocytic leukemia, chronic myeloid leukemia, hairy cell leukemia, myelodysplastic syndromes, acute promyelocytic leukemia, or myeloproliferative neoplasm.

In some exemplary embodiments, the cell population comprises a single cell type and/or subtype, a combination of cell types and/or subtypes, a cell-based therapeutic, an explant, or an organoid. In some exemplary embodiments, the cell population is a non-hematological stromal cell or cell population. In some exemplary embodiments, the cell or cell population is a MSC, OLC, bone marrow derived endothelial cell, chondrocyte, or a fibroblast cell or cell population. In some exemplary embodiments, the modulating agent is a therapeutic antibody, antibody fragment, antibody-like protein scaffold, aptamer, polypeptide, protein, genetic modifying agent, small molecule, small molecule degrader, or combination thereof. In some exemplary embodiments, the genetic modifying agent is a CRISPR-Cas system, a TALEN, a Zn-finger nuclease, or a meganuclease.

In some exemplary embodiments, described herein is an isolated or engineered mesenchymal stem/stromal cell (MSC) or MSC cell population, wherein the MSC or MSC cell population is characterized by a gene signature comprised of one or more genes of Table 1. In some exemplary embodiments, the MSC or MSC cell population is characterized by a gene signature comprised of one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4. In some exemplary embodiments, the MSC or MSC cell population does not express one or more of Thy1, Ly6a (Sca-1), NG2 (Cspg4) or Nestin (Nes). In some exemplary embodiments, the gene signature comprises one or more of Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxcl1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1.

In some exemplary embodiments, described herein is an isolated or engeinered osteolineage cell (OLC) or OLC population, where the isolated or engineered OLC or OLC population is characterized by a gene signature comprising one or more genes of Table 2. In some exemplary embodiments, the OLC or OLC population is characterized by a gene signature comprising one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1. In some exemplary embodiments, the OLC or OLC population expresses Bglap and Spp1. In some exemplary embodiments, the gene signature further comprises one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmem119, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Pre1p, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbpl11, Col3a1, Ptn, Col6a2, Tnn, Npy, Col6a1, Omd, Dcn, Tgfbi, Col6a3, or Acan. In some exemplary embodiments, the gene signature further comprises one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13.

In some exemplary embodiments, described herein is an isolated or engineered pericyte or pericyte population, wherein the isolated or engineered pericyte is characterized by a gene signature comprising one or more genes in Table 3. In some exemplary embodiments, the gene signature further comprises one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, Il6, Rarres2, Vstm4, Pgf, Pdgfa, Col4a2, Igfbp7, Col4a1, Fst, Rtn4r11, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, or Cygp. In some exemplary embodiments, the gene signature further comprises one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11. In some exemplary embodiments, the gene signature further comprises one or more Acta2, Myh11, Mcam, Jag1, and Il6.

In some exemplary embodiments, described herein is an isolated or engineered chondrocyte or chondrocyte population, wherein the isolated or engineered chondrocyte population is characterized by a gene signature comprising one or more genes in Table 4. In some exemplary embodiments, the gene signature comprises one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst11, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, Il17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a. In some exemplary embodiments, the gene signature comprises one or more of Sox9, Col11a2, Acan, or Col2a1. In some exemplary embodiments, the gene signature comprises one or more of Runx2, Ihh, Mef2c, or Col10a1. In some exemplary embodiments, the gene signature further comprises one or more of Grem1, Runx2, Sp7, Alp1, or Spp1. In some exemplary embodiments, the chondrocyte expresses one or more of Ihh, Pth1r, Mef2c, Col10a1, Ibsp, Mmp13, Grem1. In some exemplary embodiments, the gene signature comprises one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, Il17b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

In some exemplary embodiments, described herein is an isolated or engineered fibroblast or fibroblast population, wherein the isolated or engineered fibroblast or fibroblast population is characterized by a gene signature comprising one or more genes of Table 5. In some exemplary embodiments, the gene signature further comprises one or more of Scx, Barx1, Trpsl, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgb1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc. In some exemplary embodiments, the gene signature comprises one or more of Fibronectin-1 (Fn1), Fibroblast Specific Protein-1 (S100a4), Col1a1, Col1a2, Lum, Col22a1, or Twist2. In some exemplary embodiments, the gene signature comprises one or more of Sox9, Acan, and Col2a1. In some exemplary embodiments, the gene signature comprises one or more of Cd34, Ly6a, Pdgfra, Thy1 and Cd44, and not Cdh5, or Acta2. In some exemplary embodiments, the gene signature comprises one or more of Sox-9, Scleraxis (Scx), Spp1, Cspg4, CD73 (Nt5e), and Cartilage Intermediate Layer Protein (Cilp). In some exemplary embodiments, the gene signature further comprises one or more of S1004a, Dcn, Sema3c, or Cxcl12.

In some exemplary embodiments, described herein is an isolated or engineered bone marrow derived endothelial cell (BMEC) or BMEC population, wherein the isolated or engineered fibroblast or fibroblast population is characterized by a gene signature comprising one or more genes of Table 6. In some exemplary embodiments, the gene signature comprises one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x. In some exemplary embodiments, the gene signature comprises one or more of Flt4 (Vegfr-3) and Ly6a (Sca-1), wherein Ly6a expression, when present in the gene signature, is reduced as compared to a suitable control. In some exemplary embodiments, the gene signature comprises one or more of Pecam1, Cdh5, Cd34, Tek, Lepr, Cxcl12, or Kitl. In some exemplary embodiments the gene signature comprises one or more of Flt4, Ly6a, Icam1, or Sele. In some exemplary embodiments, the gene signature comprises one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

In some exemplary embodiments, described herein are methods of treating a hematological disease comprising: administering to a subject in need thereof the isolated or engineered cell or cell population as described in greater detail herein.

In some exemplary embodiments, described herein are methods of screening for one or more agents capable of modulating a stromal cell state, comprising: contacting a stromal cell population having an initial cell state with a test modulating agent or library of modulating agents, wherein the stromal cell population optionally contains leukemia cells; determining one or more fractions of stromal cell states including one or more fraction(s) of a mesenchymal stem/stromal cell (MSC), an OLC, a chondrocyte, a fibroblast, a pericyte, a bone marrow derived endothelial cell (BMEC), or a combination thereof; and selecting modulating agents that shifts the initial stromal cell state to a desired stromal cell state, wherein the desired stromal cell fraction in the stromal cell population is above a set cutoff limit. In some exemplary embodiments, determining one or more fractions of stromal cell states further comprises determining one or more MSC subtype, one or more OLC types, one or more chondrocyte types, one or more fibroblast types, one or more BMEC types, one or more pericyte subtype, or a combination thereof. In some exemplary embodiments, the stromal cell population is obtained from a subject to be treated. In some exemplary embodiments, determining one or more fractions of stromal cell states comprises identifying a MSC gene signature, an OLC gene signature, a chondrocyte gene signature, a fibroblast gene signature, a BMEC gene signature, a pericyte gene signature.

In some exemplary embodiments, the MSC gene signature comprises:

a. one or more genes of Table 1;

b. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or

c. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxcl1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1;

and wherein the MCS optionally does not express one or more of Thy1, Ly6a (Sca-1), NG2 (Cspg4) or Nestin (Nes).

In some exemplary embodiments, the OLC gene signature comprises:

a. one or more genes of Table 2;

b. one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;

c. one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmeml19, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbp11, Col3a1, Ptn, Col6a2, Tnn, Npy, Col6a1, Omd, Dcn, Tgfbi, Col6a3, or Acan;

d. one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13;

and wherein the OLC optionally expresses Bglap and Spp1.

In some exemplary embodiments, the chondrocyte gene signature comprises:

a. one or more genes of Table 4;

b. one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst1, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, I17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;

c. one or more of Sox9, Col11a2, Acan, or Col2a1;

d. one or more of Runx2, Ihh, Mef2c, or Col10a1;

e. one or more of Grem1, Runx2, Sp7, Alp1, or Spp1;

f. one or more of Ihh, Pth1r, Mef2c, Col10a1, Ibsp, Mmp13, Grem1; or

g. one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, 1117b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit4l, Egr1, Runx2, or Cxcl12.

the fibroblast gene signature comprises:

a. one or more genes of Table 5;

b. one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;

c. Fibronectin-1 (Fn1), Fibroblast Specific Protein-1 (S100a4), Col1a1, Col1a2, Lum, Col22a1, or Twist2;

d. one or more of Sox9, Acan, and Col2a1;

e. Cd34, Ly6a, Pdgfra, Thy1 and Cd44, and not Cdh5, or Acta2;

f. one or more of Sox-9, Scleraxis (Scx), Spp1, Cspg4, CD73 (Nt5e), and Cartilage Intermediate Layer Protein (Cilp); or

g. one or more of S1004a, Dcn, Sema3c, or Cxcl12.

In some exemplary embodiments, the the BMEC gene signature comprises:

a. one or more genes of Table 6;

b. one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x;

c. one or more of Flt4 (Vegfr-3) or Ly6a (Sca-1);

d. one or more of Pecam1, Cdh5, Cd34, Tek, Lepr, Cxcl12, or Kitl;

e. one or more of Flt4, Ly6a, Icam1, or Sele;

f. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, lfitm2; or

g. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

In some exemplary embodiments, the pericyte gene signature comprises:

a. one or more genes in Table 3;

b. one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, Cygp;

c. one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11; or d. one or more of Acta2, Myh11, Mcam, Jag1, or Il6.

In some exemplary embodiments, the modulating agent that shifts the initial stromal cell state to the desired stromal cell state is capable of remodeling in a hematological disease.

In some exemplary embodiments, described herein are methods of screening for one or more agents capable of modulating osteogenic and/or adipogenic differentiation in a hematological disease comprising: contacting a cell population with a test modulating agent, wherein the cell population comprises MSC(s), OLC(s), and leukemia cells; and selecting modulating agents that change the regulation of one or more of Grem1, Bmp4, Sp7, Runx2, Bglap1, Bglap2, Bglap3, Adipoq, Wisp2, Mgp, Igbfp5, Igbfp3, Mmp2, Mmp11, or Mmp13.

In some exemplary embodiments, described herein are methods of screening for one or more agents capable of remodeling in a hematological disease comprising:

contacting a cell population with a test modulating agent, wherein the cell population comprises MSC(s), OLC(s), and leukemia cells; and

selecting modulating agents that

a. change the proportion of prerosteoblasts in the cell population;

b. change the relative proportion of OLC-1 to OLC-2 in the cell population;

c. change the relative proportion of hypertrophic chondrocytes to progenitor chondrocytes in the cell population;

d. change the relative proportion of subtype-3 (Cluster 16) fibroblasts to subtype-4 fibroblasts (Cluster 3); or

e. a combination thereof.

In some exemplary embodiments, described herein are methods of detecting a mesenchymal stem/stromal cell (MSC) from a population of stromal cells comprising:

detecting in a sample the expression or activity of a MSC gene expression signature,

wherein detection of the MSC gene expression signature indicates MSCs in the sample, and

wherein the MSC gene expression signature comprises:

a. one or more genes of Table 1;

b. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or

c. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxcl1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1;

and wherein the MCS optionally does not express one or more of Thy1, Ly6a (Sca-1), NG2 (Cspg4) or Nestin (Nes).

In some exemplary embodiments, described herein are methods of detecting an osteolineage cell (OLC) from a population of stromal cells comprising:

detecting in a sample the expression or activity of an OLC gene expression signature,

wherein detection of the OLC gene expression signature indicates OLCs in the sample, and

wherein the OLC gene expression signature comprises

a. one or more genes of Table 2;

b. one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;

c. one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmeml19, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbp11, Col3a1, Ptn, Col6a2, Tnn, Npy, Col6a1, Omd, Dcn, Tgfbi, Col6a3, or Acan;

d. one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13];

and wherein the OLC optionally expresses Bglap and Spp1.

In some exemplary embodiments, described herein are methods of detecting a chondrocyte from a population of stromal cells comprising:

detecting in a sample the expression or activity of a chondrocyte gene expression signature,

wherein detection of the chondrocyte gene expression signature indicates chondrocytes in the sample, and

wherein the chondrocyte gene expression signature comprises

a. one or more genes of Table 4;

b. one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst1, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, Il17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;

c. one or more of Sox9, Col11a2, Acan, or Col2a1;

d. one or more of Runx2, Ihh, Mef2c, or Col10a1;

e. one or more of Grem1, Runx2, Sp7, Alp1, or Spp1;

f. one or more of Ihh, Pth1r, Mef2c, Col10a1, Ibsp, Mmp13, Grem1; or

g. one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, Il17b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

In some exemplary embodiments, described herein are methods of detecting a fibroblast from a population of stromal cells comprising:

detecting in a sample the expression or activity of a fibroblast gene expression signature,

wherein detection of the fibroblast gene expression signature indicates fibroblasts in the sample, and

wherein the fibroblast gene expression signature comprises

a. one or more genes of Table 5;

b. one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;

c. Fibronectin-1 (Fn1), Fibroblast Specific Protein-1 (S100a4), Col1a1, Col1a2, Lum, Col22a1, or Twist2;

d. one or more of Sox9, Acan, and Col2a1;

e. Cd34, Ly6a, Pdgfra, Thy1 and Cd44, and not Cdh5, or Acta2;

f. one or more of Sox-9, Scleraxis (Scx), Spp1, Cspg4, CD73 (Nt5e), and Cartilage Intermediate Layer Protein (Cilp); or

g. one or more of S1004a, Dcn, Sema3c, or Cxcl12.

In some exemplary embodiments, described herein are methods of detecting a bone marrow derived endothelial cell (BMEC) from a population of stromal cells comprising:

detecting in a sample the expression or activity of a BMEC gene expression signature,

wherein detection of the BMEC gene expression signature indicates BMECs in the sample, and

wherein the fibroblast gene expression signature comprises

a. one or more genes of Table 6;

b. one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x;

c. one or more of Flt4 (Vegfr-3) or Ly6a (Sca-1);

d. one or more of Pecam1, Cdh5, Cd34, Tek, Lepr, Cxcl12, or Kitl;

e. one or more of Flt4, Ly6a, Icam1, or Sele;

f. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, lfitm2; or

g. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

In some exemplary embodiments, described herein are methods of detecting a pericyte from a population of stromal cells comprising:

detecting in a sample the expression or activity of a pericyte gene expression signature,

wherein detection of the pericyte gene expression signature indicates pericyte s in the sample, and

wherein the fibroblast gene expression signature comprises

a. one or more genes in Table 3;

b. one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, Cygp;

c. one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11; or

d. one or more of Acta2, Myh11, Mcam, Jag1, or Il6.

In some exemplary embodiments, the sample is obtained from the blood or bone marrow.

In some exemplary embodiments, described herein are methods of preparing a mesenchymal stem/stromal cell (MSC) enriched cell population a stromal cell population comprising:

enriching the population of stromal cells for cells that have an MSC gene signature, wherein the gene signature comprises

a. one or more genes of Table 1;

b. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or

c. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxcl1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1;

and wherein the MCS optionally does not express one or more of Thy1, Ly6a (Sca-1), NG2 (Cspg4) or Nestin (Nes).

In some exemplary embodiments, described herein are methods of preparing an osteolineage (OLC) enriched cell population a stromal cell population comprising:

enriching the population of stromal cells for cells that have an OLC gene signature, wherein the gene signature comprises

a. one or more genes of Table 2;

b. one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;

c. one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmeml19, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbp11, Col3a1, Ptn, Col6a2, Tnn, Npy, Col6a1, Omd, Dcn, Tgfbi, Col6a3, or Acan;

d. one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13;

and wherein the OLC optionally expresses Bglap and Spp1.

In some exemplary embodiments, described herein are methods of preparing a chondrocyte enriched cell population a stromal cell population comprising:

enriching the population of stromal cells for cells that have a chondrocyte gene signature, wherein the gene signature comprises

a. one or more genes of Table 4;

b. one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst1, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, Il17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;

c. one or more of Sox9, Col11a2, Acan, or Col2a1;

d. one or more of Runx2, Ihh, Mef2c, or Col10a1;

e. one or more of Grem1, Runx2, Sp7, Alp1, or Spp1;

f. one or more of Ihh, Pth1r, Mef2c, Col10a1, Ibsp, Mmp13, Grem1; or

g. one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, 117b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

In some exemplary embodiments, described herein are methods of preparing a fibroblast enriched cell population a stromal cell population comprising:

enriching the population of stromal cells for cells that have a fibroblast gene signature, wherein the gene signature comprises

a. one or more genes of Table 5;

b. one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;

c. Fibronectin-1 (Fn1), Fibroblast Specific Protein-1 (S100a4), Col1a1, Col1a2, Lum, Col22a1, or Twist2;

d. one or more of Sox9, Acan, and Col2a1;

e. Cd34, Ly6a, Pdgfra, Thy1 and Cd44, and not Cdh5, or Acta2;

f. one or more of Sox-9, Scleraxis (Scx), Spp1, Cspg4, CD73 (Nt5e), and Cartilage Intermediate Layer Protein (Cilp); or

g. one or more of S1004a, Dcn, Sema3c, or Cxcl12.

In some exemplary embodiments, described herein are methods of preparing a bone marrow derived endothelial cell (BMEC) enriched cell population a stromal cell population comprising:

enriching the population of stromal cells for cells that have a BMEC gene signature, wherein the gene signature comprises

a. one or more genes of Table 6;

b. one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x;

c. one or more of Flt4 (Vegfr-3) or Ly6a (Sca-1);

d. one or more of Pecam1, Cdh5, Cd34, Tek, Lepr, Cxcl12, or Kitl;

e. one or more of Flt4, Ly6a, Icam1, or Sele;

f. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, lfitm2; or

g. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

In some exemplary embodiments, described herein are methods of preparing a pericyte enriched cell population a stromal cell population comprising:

enriching the population of stromal cells for cells that have a pericyte gene signature, wherein the gene signature comprises

a. one or more genes in Table 3;

b. one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, Cygp;

c. one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, Col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11; or

d. one or more of Acta2, Myh11, Mcam, Jag1, or Il6.

In some exemplary embodiments, enriching the population of stromal cells comprises determining an MSC, an OLC, a chondrocyte, a BMEC, a fibroblast, a pericyte gene signature, or a combination thereof, wherein the gene signature(s) are determined by single cell RNA sequencing.

In some exemplary embodiments, described herein are methods of detecting a hematological disease comprising:

a. determining a fraction of:
i. OLC-1 cells,
ii. OLC-2 cells,
iii. bone marrow derived endothelial cells (BMECs);
iv. chondrocytes;
v. fibroblasts; and

b. diagnosing the neurodegenerative disease in the subject when
i. the relative proportion of OLC-1 cells to OLC-2 cells is changed as compared to a suitable control;
ii. the fraction of OLC-1 cells is increased as compared to a suitable control;
iii. the fraction of OLC-2 cells is decreased as compared to a suitable control;
iv. the relative proportion of bone marrow derived endothelial fractions is changed as compared to a suitable control;
v. a fraction of sinusoidal BMECs is decreased as compared to a suitable control;
vi. a fraction of arterial BMECs is increased as compared to a suitable control;
vii. the relative proportion of chondrocyte fractions is changed as compared to a suitable control;
viii. a chondrocyte hypertorphic cell subtype is increased as compared to a suitable control;
ix. a chondrocyte progenitor cell subtype is decreased as compared to a suitable control;
x. a fibroblast subtype is changed as compared to a suitable control;
xi. a fibroblast subtype-3 is decreased; as compared to a suitable control
xii. a fibroblast subtype-4 is increased as compared to a suitable control;
xiii. the relative proportion of MSC fractions is changed as compared to a suitable control;
ixx. a MSC-2 fraction is increased as compared to a suitable control;
xx. a MSC-3 fraction is decreased as compared to a suitable control;
xxi. a MSC-4 fraction is decreased as compared to a suitable control; or
xxii. a combination thereof.

In some exemplary embodiments, the hematological disease is a blood cancer. In some exemplary embodiments, the blood cancer is a leukemia. In some exemplary embodiments, the blood cancer is acute lymphocytic leukemia, acute myeloid leukemia, chronic lymphocytic leukemia, chronic myeloid leukemia, hairy cell leukemia, myelodysplastic syndrome, acute promyelocytic leukemia, or myeloproliferative neoplasm.

In some exemplary embodiments, described herein are methods of treating a hematological disease in a subject in need thereof, comprising: detecting a hematological disease as in a subject according a method of detecting a hematological disease described herein and administering an effective amount of a hematological disease treatment to the subject.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1H—FIG. 1A) Overview of Single Cell analysis. Bone marrow (BM) cells were isolated, hematopoietic cells were filtered out by cell sorting (FACS). Single cell transcriptomes were generated with 10× GemCode™ platform and analyzed. FIG. 1B) BM stroma cells clusters visualized with t-SNE maps. FIG. 1C) BM stroma maps (as in FIG. 1B) with expression (as shown) of six genes that are broadly characteristic to six major cell types. FIG. 1D) Relative expression of top differential genes in clusters—single cell resolution (largest clusters down-sampled for visibility). Right—characteristic genes. Top bar and vertical lines indicate clusters (FIG. 1B). FIG. 1E) Number of cells in each cluster. FIG. 1F) Correlation (Pearson) of average gene expression among clusters (marked by side bars). FIG. 1G) Cluster graph abstraction (AGA) from Scanpy package. FIG. 1H) Single-cell diffusion map visualization (force-directed layout) of strongly connected clusters (from FIGS. 1G-F)).

FIGS. 2A-2K—FIG. 2A) Gating strategy for isolation of bone marrow stroma. FIG. 2B) RNA-seq data quality measures (UMI and genes in cells). FIG. 2C) t-SNE map of the bone marrow (BM) cells (hematopoietic and non-hematopoietic) (obtained as in FIG. 2A). FIG. 2D) As in FIG. 2C but with hematopoietic clusters markers avg. expression marked in dark gray. FIG. 2E) As in FIG. 2C but with colors marking samples. FIG. 2F) All cluster single cell diffusion map visualization (force-directed layout, clusters indicated by shades of gray). FIG. 2G) (left) t-SNEs (as in FIG. 1B) of select genes from MSC and EC (gray scale—transcript level in TP10K). (right) corresponding distributions in logarithmic scale. FIG. 2H) Number of cells belonging to major cell types. FIG. 2I) Cell proliferation status marked on t-SNE maps of BM stroma (red—high expression of cell-cycle genes). FIGS. 2J-2K) FACS analysis of MSC and EC clusters.

FIGS. 3A-3F—FIG. 3A) MSC (cluster-1), location in BM stroma map. FIG. 3B) (left) Expression maps of select MSC genes; (right) Expression distributions in logarithmic scale (violin plots by clusters). FIG. 3C) Top MSC differential genes (relative transcription level, averaged per cluster) in five gene categories: characteristic—Known, transcription factors (TFs), found in cell membrane (Surface), Secreted, and Other. FIG. 3D) MSC sub-clusters on diffusion map (as in FIG. 1H). FIG. 3E) MSC sub-clusters (zoomed-in original t-SNE map. FIG. 3F) Expression distributions of select genes in MSC sub-clusters (linear scale, censored).

FIGS. 4A-4F—FIG. 4A) t-SNE maps of bone marrow stroma with color coded transcription levels (TP10K) of additional characteristic genes used by various research groups for labeling mesenchymal stem cell populations. FIG. 4B) as in FIG. 4A, additional genes. FIG. 4C) Average expression (ln(TP10K+1)) in all BM stroma clusters of top secreted genes that were also differentially expressed in MSCs (cluster 1). D) MSC sub-clusters diffusion map (2D projection, eigenvectors 1 and 3). FIG. 4E) Top differentially expressed genes among MSC sub-clusters (single cell view, relative expression z-score). FIG. 4F) Expression distributions of select genes in MSC sub-clusters (linear scale, censored).

FIGS. 5A-5M—FIG. 5A) OLC-1 (cluster 7) and OLC-2 (cluster 8) locations in BM stroma map. FIG. 5B) expression maps of characteristic OLC-1 genes. FIG. 5C) Top OLC-1 differential genes (relative transcription level, averaged per cluster). FIG. 5D) OLC-1,2 sub-clusters on diffusion map (as in FIG. 1H). FIG. 5E) OLC-1,2 sub-clusters (zoomed-in original t-SNE map). FIGS. 5F-5J) Expression distributions of select genes in OLC-1 sub-clusters (linear scale, censored). FIG. 5K) OLC-2 sub-clusters (zoomed-in original t-SNE map). FIGS. 51-5M) Expression distributions of select genes in OLC-2 sub-clusters (linear scale, censored).

FIGS. 6A-6E—FIG. 6A) Expression distributions of select genes in logarithmic scale (violin plots, clusters identity indicated on x-axis and by color as in main FIG. 1B) FIG. 6B). Expression of select genes marked on t-SNE maps of BM stroma. FIG. 6C) Top differentially expressed genes among OLC (cluster 7) sub-clusters (single cell view, relative expression z-score).

FIG. 6D) Top differentially expressed genes among cluster 8 sub-clusters (single cell view, relative expression z-score). FIG. 6E) Expression distributions of select genes in cluster 8 sub-clusters (linear scale, censored).

FIGS. 7A-7G—FIG. 7A) Five chondroid clusters in BM stroma map. FIG. 7B) (left) Expression maps of characteristic chondrocyte genes; (right) expression distributions in logarithmic scale. FIG. 7C) Chondroid clusters on diffusion map. FIG. 7D) Five fibroblastic clusters in BM stroma map. FIG. 7E) (left) Expression maps of fibroblast characteristic genes; (right) expression distributions in logarithmic scale. FIG. 7F) Fibroblastic clusters on diffusion map. FIG. 7G) Expression distribution of Cxcl12 among fibroblastic clusters (logarithmic scale).

FIGS. 8A-8F—FIG. 8A) Top chondrocyte differential genes (relative transcription level, averaged per cluster) in five categories: known, transcription factors (TFs), cell surface, secreted, and other. FIG. 8B) Expression maps of select chondrocyte related genes. FIG. 8C) Top differentially expressed genes among chondrocyte clusters (single cell view, relative expression z-score). FIG. 8D) As in A but for fibroblast clusters. FIGS. 8E-8F) Expression maps and expression distributions (logarithmic scale) of select genes related to fibroblastic clusters.

FIGS. 9A-9E—FIG. 9A) Three endothelial cell (EC) clusters in BM stroma map. FIG. 9B) (left) Expression maps of select characteristic EC genes; (right) expression distributions in logarithmic scale. FIG. 9C) Top EC differential genes (relative transcription level, averaged per cluster). FIG. 9D) EC clusters diffusion map on force-directed layout. FIG. 9E) Expression distributions of select genes in EC clusters (linear scale, censored).

FIGS. 10A-10C—FIG. 10A) (left) expression maps of select characteristic EC genes; (right) expression distributions in logarithmic scale. FIG. 10B). Top differentially expressed genes among three EC clusters (single cell view, relative expression z-score). FIG. 10C) Expression distributions in logarithmic scale of Tek.

FIGS. 11A-11F—FIG. 11A) Pericyte cluster in BM stroma map. FIG. 11B) (left) Expression maps of characteristic pericyte genes; (right) expression distributions in logarithmic scale. FIG. 11C) Top pericyte differential genes (relative transcription level, averaged per cluster). FIG. 11D) Pericyte sub-clusters (zoomed-in original t-SNE map. FIG. 11E) Average expression (ln(TP10K+1)) of select hematopoietic niche genes. Three pericyte sub-clusters indicated. FIG. 11F) Expression distributions of select genes in pericyte sub-clusters (linear scale, censored).

FIGS. 12A-12C—FIG. 12A) (left) expression maps of select characteristic pericyte genes; (right) expression distributions in logarithmic scale. FIG. 12B) Top differentially expressed genes among pericyte sub-clusters (single cell view, relative expression z-score). FIG. 12C) As in FIG. 12A, for additional genes.

FIGS. 13A-13K-Control and Leukemic BM stroma t-SNE map—FIG. 13A) clusters assignment (colors as in FIG. 1B), FIG. 13B) Control (light grey) and Leukemic (dark grey) cells colored. FIG. 13C) Changes of BM stroma relative cluster sizes under leukemia. Bars—average percentage of cells in cluster. Error bars−95% confidence interval of the binomial fit mean. FIG. 13D) As in FIG. 13C but for MSC and OLC sub-clusters. FIG. 13E) Average transcription of Grem1 gene in MSC and OLC sub-clusters. FIG. 13F) Changes in average transcription of BM niche modelling genes (MSC and OLC-1). Error bars for standard errors of mean. FIG. 13G) As in FIG. 13F for but for hypoxia factor Hif-2a (Epasl). FIG. 131H) As in FIG. 13F but for hematopoietic regulators (MSC, OLC-1, sBMEC). I) As in FIG. 13G but comparing changing expression of Cxcl2 and Kitl in sBMEC, aBMEC, progenitor EC (cluster 11) and pericytes. FIG. 13J) As in FIG. 13G but looking at changes of Cxcl12, Kit1, and Angpt1 in MSC-like fibroblasts (cluster 9). FIG. 13K) Summary plot. Each horizontal rectangle corresponds to a cell type. Sub-cluster names next to cell-type symbolic representations. Circles mark expression levels of key niche factors (Kitl and Cxcl12). Dark triangles indicate changes in size of clusters (sub-clusters) under Leukemia. Colored triangles indicate changes in relative expression under leukemia. ‘C’-mark next to triangles indicate cluster level change (i.e. refers to all cells in a rectangle).

FIGS. 14A-14J—FIG. 14A) Spleen weight of leukemic mice used for single-cell RNA-sequencing experiments. Spleen weights for matched controls (n=5) and leukemic mice (n=4). (student t-test, *p<0.05). FIG. 14B) Donor chimerism and leukemic blast appearance. FIG. 14C) Frequency of myeloid cells in peripheral blood from control and leukemic mice characterized by FACS analysis (FIG. 14B). FIG. 14D) t-SNE map of leukemic and control data (as in FIGS. 7A-7B), samples colored. FIG. 14E) Relative cluster sizes of leukemic and control samples in log scale. Bars for mean percentage of cells. Error bars for 95% confidence interval of the mean estimate. FIG. 14F) Significant, differentially transcribed, secreted genes (adjusted p-value <0.05) up- (red) and down- (blue) regulated in leukemia). Dot size proportional to absolute value of the base-2 log of fold-change. FIG. 14G) As in F but for cell surface expressed genes. FIG. 14H) As in F but for transcription factor coding genes. FIG. 14I) Average transcription of Wisp2 gene in MSC and OLC sub-clusters. FIG. 14J) Changes in average transcription of Hifla gene in MSC (cluster 1) and OLC-1 (cluster 7). Error bars for standard errors

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−20%, +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”. It is noted that in this disclosure and particularly in the claims and/or paragraphs, terms such as “comprises”, “comprised”, “comprising” and the like can have the meaning attributed to it in U. S. Patent law; e.g., they can mean “includes”, “included”, “including”, and the like; and that terms such as “consisting essentially of” and “consists essentially of” have the meaning ascribed to them in U. S. Patent law, e.g., they allow for elements not explicitly recited, but exclude elements that are found in the prior art or that affect a basic or novel characteristic of the invention.

The recitation of numerical ranges by endpoints includes all numbers and fractions subsumed within the respective ranges, as well as the recited endpoints.

Whereas the terms “one or more” or “at least one” or “X or more”, where X is a number and understand to mean X or increases one by one of X, such as one or more or at least one member(s) or “X or more” of a group of members, is clear per se, by means of further exemplification, the term encompasses inter alia a reference to any one of said members, or to any two or more of said members, such as, e.g., any >3, >4, >5, >6 or >7 etc. of said members, and up to all said members.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide various signatures, profiles, programs, and/or modules, that can be unique bone marrow stromal cell types, subtypes, states, and remodeling of the bone marrow microenvironment. The various signatures, profiles, programs, and/or modules unique bone marrow stromal cell types, subtypes, states, and remodeling of the bone marrow microenvironment can be used to identify and characterize specific cell populations. Thus, also described herein are bone marrow stromal cell populations that can be uniquely characterized, isolated, enriched for, and/or engineered to have and/or express a cell-state and/or cell type/subtype specific signature, profile, module, and/or program. Also described herein are isolated, enriched, modulated and/or engineered bone marrow stromal cell populations. The modulated and engineered cells can be modulated using a suitable modulating agent to express specific signatures, profiles, programs, and/or modules(s), such as those described here unique to any one of Clusters 1-17, or a subtype thereof, where the initial cell type or state of the cell before modulation or engineering is different than after exposure to the modulating agent.

Also described herein are methods of detecting the stromal cell signatures, profiles, programs, and/or modules described herein. The methods of detecting the stromal cell signatures can be used in methods of diagnosing and treatment. In some embodiments, the methods can include detecting one or more stromal cell signatures, profiles, programs, and/or modules and treating and/or diagnosing a subject based on the presence, absence, or change in one or more particular stromal cell signature, profile, program, and/or module. Also described herein are methods of treating that include administering a modulating agent to subject. In some embodiments the modulating agent can alter in vivo the type and/or state of a stromal cell. In some embodiments, modulated cells can be generated ex vivo and administered to a subject in need thereof to enhance the presence of a desired cell population in the subject.

Also described herein are methods of modulating cells and methods of screening modulating agents.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Bone Marrow Stromal Cell Populations

Described herein are bone marrow stromal cells (also referred to herein as simply “stromal cells”) that can be uniquely characterized, isolated, enriched for, and/or engineered to have and/or express a cell-state and/or cell type/subtype specific signature, profile, module, and/or program.

Biomarkers, signatures and molecular targets described herein can be associated with the bone marrow microenvironment, immune cell dysfunction, and/or activation. In some embodiments, some of the biomarkers, signatures, and/or molecular targets described herein correlate with the loss of effector function of the immune cells and are advantageously distinct, separate or uncoupled from, or independent of the immune cell activation status. In some embodiments, one or more of the biomarkers, marker signatures and molecular targets correlate with immune cell activation and are advantageously distinct, separate or uncoupled from, or independent of the immune cell dysfunction status. As described elsewhere herein, gene signatures and/or gene modules that are uniquely associated with cell types and subtypes, including in normal and in dysfunctional cell states, and molecular nodes that control them and can be analyzed and can uniquely identify a particular cell state (e.g. normal or dysfunctional) and/or type. In some embodiments, the biomarkers, signatures, and/or molecular targets described herein can be used to evaluate bone marrow microenvironments and response, such as to specifically evaluate and target a dysfunctional state while leaving normal activation programs intact.

As used herein, “cell state” is used to describe elements of a cell's identity. Cell state can be thought of as the characteristic profile or phenotype of a cell, which can be transient or permanent. Cell states can arise transiently during a process that can occur over a period of time. Temporal progression from one cell state to another can be unidirectional (e.g., during differentiation, or following an environmental stimulus) or can be in a state of vacillation that is not necessarily unidirectional and in which the cell may return to the origin state. Vacillating processes can be oscillatory (e.g., cell-cycle or circadian rhythm) or can transition between states with no predefined order (e.g., due to stochastic, or environmentally controlled, molecular events). These processes may occur transiently within a stable cell type (such as in a transient environmental response), or may lead to a new, distinct type (such as in differentiation). Wagner et al., 2016. Nat Biotechnol. 34(111): 1145-1160.

### Bone Marrow Stromal Cell Signatures

Described herein are distinct cell populations that can be identified within a bone marrow stromal cell population by the unique signature of the specific bone marrow cell population.

As used herein a signature may encompass any gene or genes, or protein or proteins, whose expression profile or whose occurrence is associated with a specific cell type, subtype, or cell state of a specific cell type or subtype within a population of cells. Increased or decreased expression or activity or prevalence may be compared between different cells in order to characterize or identify for instance specific cell (sub)populations. A gene signature as used herein, may thus refer to any set of up- and down-regulated genes between different cells or cell (sub)populations derived from a gene-expression profile. For example, a gene signature may comprise a list of genes differentially expressed in a distinction of interest. It is to be understood that also when referring to proteins (e.g. differentially expressed proteins), such may fall within the definition of “gene” signature.

The signatures as defined herein (being it a gene signature, protein signature or other genetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. blood samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of immune cells that are linked to particular pathological condition (e.g. cancer), or linked to a particular outcome or progression of the disease, or linked to a particular response to treatment of the disease.

The signature according to certain embodiments of the present invention may comprise or consist of one or more genes and/or proteins, such as for instance 1, 2, 3, 4, 5, 6,7, 8, 9, 10, 11, 12, 13, 14, 15, 16, 17, 18, 19, 20, 21, 22, 23, 24, 25, 26, 27, 28, 29, 30, 31, 32,33, 34, 35, 36, 37, 38, 39, 40, 41, 42, 43, 44, 45, 46, 47, 48, 59, or 50 or more. In certain embodiments, the signature may comprise or consist of two or more genes and/or proteins, such as for instance 2, 3, 4, 5, 6, 7, 8, 9, 10, 11, 12, 13, 14, 15, 16, 17, 18, 19, 20, 21, 22, 23,24, 25, 26, 27, 28, 29, 30, 31, 32, 33, 34, 35, 36, 37, 38, 39, 40, 41, 42, 43, 44, 45, 46, 47, 48,59, or 50 or more. In certain embodiments, the signature may comprise or consist of three or more genes and/or proteins, such as for instance 3, 4, 5, 6, 7, 8, 9, 10, 11, 12, 13, 14, 15, 16,17, 18, 19, 20, 21, 22, 23, 24, 25, 26, 27, 28, 29, 30, 31, 32, 33, 34, 35, 36, 37, 38, 39, 40, 41,42, 43, 44, 45, 46, 47, 48, 59, or 50 or more. In certain embodiments, the signature may comprise or consist of four or more genes and/or proteins, such as for instance 4, 5, 6, 7, 8, 9,10, 11, 12, 13, 14, 15, 16, 17, 18, 19, 20, 21, 22, 23, 24, 25, 26, 27, 28, 29, 30, 31, 32, 33, 34,35, 36, 37, 38, 39, 40, 41, 42, 43, 44, 45, 46, 47, 48, 59, or 50 or more. In certain embodiments, the signature may comprise or consist of five or more genes and/or proteins, such as for instance 5, 6, 7, 8, 9, 10, 11, 12, 13, 14, 15, 16, 17, 18, 19, 20, 21, 22, 23, 24, 25,26, 27, 28, 29, 30, 31, 32, 33, 34, 35, 36, 37, 38, 39, 40, 41, 42, 43, 44, 45, 46, 47, 48, 59, or 50 or more. In certain embodiments, the signature may comprise or consist of six or more genes and/or proteins, such as for instance 6, 7, 8, 9, 10, 11, 12, 13, 14, 15, 16, 17, 18, 19, 20,21, 22, 23, 24, 25, 26, 27, 28, 29, 30, 31, 32, 33, 34, 35, 36, 37, 38, 39, 40, 41, 42, 43, 44, 45,46, 47, 48, 59, or 50 or more. In certain embodiments, the signature may comprise or consist of seven or more genes and/or proteins, such as for instance 7, 8, 9, 10, 11, 12, 13, 14, 15, 16,17, 18, 19, 20, 21, 22, 23, 24, 25, 26, 27, 28, 29, 30, 31, 32, 33, 34, 35, 36, 37, 38, 39, 40, 41,42, 43, 44, 45, 46, 47, 48, 59, or 50 or more. In certain embodiments, the signature may comprise or consist of eight or more genes and/or proteins, such as for instance 8, 9, 10 or more. In certain embodiments, the signature may comprise or consist of nine or more genes and/or proteins, such as for instance 9, 10, 11, 12, 13, 14, 15, 16, 17, 18, 19, 20, 21, 22, 23,24, 25, 26, 27, 28, 29, 30, 31, 32, 33, 34, 35, 36, 37, 38, 39, 40, 41, 42, 43, 44, 45, 46, 47, 48,59, or 50 or more. In certain embodiments, the signature may comprise or consist of ten or more genes and/or proteins, such as for instance 10, 11, 12, 13, 14, 15, 16, 17, 18, 19, 20, 21,22, 23, 24, 25, 26, 27, 28, 29, 30, 31, 32, 33, 34, 35, 36, 37, 38, 39, 40, 41, 42, 43, 44, 45, 46,47, 48, 59, or 50 or more.

Described herein are genes and gene products differentially upregulated or downregulated in stromal cells, which thus provide useful markers, marker signatures and molecular targets specifically for stromal cells. In some embodiments, a signature can include a combination of genes of Table 1, Table 2, Table 3, Table 4, Table 5, Table 6, Table 7, and/or Table 8. It is to be understood that a signature according to the invention can, for instance, also include a combination of genes or proteins.

It is to be understood that “differentially expressed” genes/proteins include genes/proteins which are up- or down-regulated as well as genes/proteins which are turned on or off. When referring to up- or down-regulation, in certain embodiments, such up- or downregulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a negative control cell or than an average signal generated for the marker by a population of negative control cells, e.g., at least 2-fold, at least 4-fold, at least 10-fold, at least 20-fold, at least 30-fold, at least 40-fold, at least 50-fold higher or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of negative control cells. The upregulation and/or downregulation of gene or gene product, including the amount, may be included as part of the gene signature or expression profile.

A “deviation” of a first value from a second value may generally encompass any direction (e.g., increase: first value >second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±×SD or ±2×SD or ±3×SD, or ±×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

As discussed herein, differentially expressed genes/proteins may be differentially expressed on a single cell level, or may be differentially expressed on a cell population level. Preferably, the differentially expressed genes/proteins as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein of the signature, such as for instance at least to, at least three, at least four, at least five, at least six, or all genes/proteins of the signature.

Signatures may be functionally validated as being uniquely associated with a particular immune phenotype. Induction or suppression of a particular signature may consequentially be associated with or causally drive a particular immune phenotype.

In various embodiments and described in greater detail elsewhere herein signatures (e.g. gene signatures, protein signature, and/or other genetic signature) can be analyzed based on single cell analyses (e.g. single cell RNA sequencing) or alternatively based on cell population analyses, as is defined herein elsewhere.

As used herein the term “signature gene” used interchangeably with “gene signature” refers to any gene or genes whose expression profile is associated with a specific cell type, subtype, or cell state of a specific cell type or subtype within a population of cells. The signature gene(s) can be used to indicate the presence of a cell type, a subtype of the cell type, the state of the microenvironment of a population of cells, and/or the overall status of the entire cell population. Furthermore, the signature gene(s) can be indicative of cells within a population of cells in vivo. Not being bound by a theory, the signature gene(s) can be used to deconvolute the cells present in a tumor based on comparing them to data from bulk analysis of a tumor sample. The signature gene(s) can indicate the presence of one particular cell type or subtype. In one embodiment, the signature gene(s) can indicate that dysfunctional or activated tumor infiltrating T-cells are present. The presence of cell types within a tumor may indicate that the tumor will be resistant to a treatment. In one embodiment, the signature gene(s) of the present invention are applied to bulk sequencing data from a tumor sample to transform the data into information relating to disease outcome and personalized treatments. In one embodiment, the signature gene(s) can be used to detect multiple cell states that occur in a subpopulation of tumor cells that are linked to resistance to targeted therapies and progressive tumor growth. In some embodiments, immune cell states of tumor infiltrating lymphocytes are detected.

The signature gene(s) can be detected by immunofluorescence, mass cytometry (CyTOF), FACS, drop-seq, RNA-seq, single cell qPCR, MERFISH (multiplex (in situ) RNA FISH), microarray and/or by in situ hybridization. Other methods including, but not limited to, absorbance assays and colorimetric assays are known in the art and can be used herein. In some embodiments, measuring expression of signature genes can include measuring protein expression levels. Protein expression levels can be measured, for example, by performing a Western blot, an ELISA or binding to an antibody array. In another aspect, measuring expression of said genes comprises measuring RNA expression levels. RNA expression levels may be measured by performing RT-PCR, Northern blot, an array hybridization, or RNA sequencing methods. Methods of detecting a signature, such as a gene signature, are described in greater detail elsewhere herein.

Signatures may be functionally validated as being uniquely associated with a particular immune phenotype. Induction or suppression of a particular signature may consequentially be associated with or causally drive a particular immune or other desired phenotype.

Systematic characterization of non-hematopoietic cells of the mouse bone marrow, as demonstrated in the Working Examples and described elsewhere herein, provides for classification into various cell types, six broad cell types with 17 cell subsets, with discrete distinctions, differentiation continuums and HSC niche regulatory function. Each of the subsets is characterized by numerous differentially expressed genes, including but not limited to transcription factors, surface antigens, and secreted products. The differentially expressed genes include certain “known” genes, that is genes whose expression has previously been indicated to be associated with certain cell types, but which are insufficient to draw the distinctions between cell populations demonstrated, described, and provided herein. The cell types comprise mesenchymal stromal cells (MSC), osteolineage cells, chondrocytes, endothelial cells, and pericytes. The following tables provide genes showing the greatest differential expression in the various distinct bone marrow stromal cell clusters and can be used to characterize and identify distinct bone marrow stromal cell types and subtypes. While the expression patterns confirm differential expression of certain “known” genes for certain cell types, those genes may also be differentially expressed in other cell types. That is, for example, while differential expression of certain genes may be associated with MSCs, differential expression of those genes is also observed among clusters other than cluster 1. Further, the Working Examples herein can demonstrate that expression patterns of the differentially expressed genes can be used to uniquely identify distinct bone marrow stromal cell types and subtypes. Unexpected subtypes of cells found within these cell groups include two types of osteoblasts, four chondrocyte populations and three types of endothelial cells.

The distinct profiles of the cell subsets notably include hematopoietic regulatory genes, indicated participation in hematopoietic regulation, often disrupted by the emergence of leukemia.

TABLE 1
Differentially expressed genes in cluster 1 (MSCs)

| | |
|---|---|
| “known” | Cxcl12, Adipoq, Kitl, Lepr |
| Transcription | Cebpa, Zeb2, Runx2, Ebf1, Foxc1, Cebpb, |
| Factors | Ar, Fos, Id4, Klf6, Irf1, Runx2, Jun, Snaj2, |
| | Maf, Zthx4, Id3, Egr1, Junb |
| Surface | Hp, Lpl, Gdpd2, Serping, Dpep1, Grem1, |
| | Pappa, Chrdl1, Fbln5, Vcam1, Kng1, |
| | H2-Q10, Cdh11, Mme, Tmem176b, Csf1, |
| | H2-K1, Serpine2, H2-D1, Tnc, Cdh2, Pdgtra |
| Secreted | Esm1, Gas6, Cxcl14, Sfrp4, Wisp2, Agt, |
| | Il34, Fst, Fgf7, Il1rn, C2, Igfpb4, Serpina1, |
| | Cb1n1, Apoe, Ibsp, Igfbp5, Gpx3, Pdzrn4, |
| | Rarres2, Vegfa |
| Other | 1500009L16Rik, Serpina3g, Cyp1b1, Ebt3, |
| | Arrdc4, Kng2, Slc26a7, Marc1, Ms4ad4, |
| | Wdr86, Serpina3c, Tmem176a, Cldn10, Trt, |
| | Gpr88, Nnmt, Gm4951, Cd1d1, Plpp3, Ackr4 |

TABLE 2
Differentially expressed genes in cluster 7, 8 (OLCs)

| | |
|---|---|
| “known” | Bglap, Spp1 |
| Transcription | Vdr, Satb2, Sp7, Runx2, Tbx2, Zeb2, Dlx5, |
| Factors | Dlx6, Zfhx4, Hey1, Irx5, Id3, Mxd4, Mef2c, |
| | Esr1, Maf, Smad6, Sox4, Cebpb, Meis3 |
| Surface | Mmp13, Tnc, Cfh, Alp1, Lrp4, Cdh11, |
| | Casm1, Cdh2, Slit2, Bmp3, Cdh15, Fat3, |
| | Pard6g, Litr, Cp, Ptprd, Olfml3 Fign, Cd63, |
| | Fap |
| Secreted | Dmp1, Angpt4, Chn1, Ibsp, Wisp1, Wif1, |
| | Metrnl, Vldlr, Podnl1, Col22a1, Ndnf, |
| | Mmp14, Pgf, Lox11, Mfap2, Srpx2, Ag-t, |
| | Tmem59, Vstm4, Col8a1, Cxcl12 |
| Other | Bglap2, Car3, Kcnk2, Slc36a2, Ifitm5, |
| | Hpgd, Limch1, Gm44029, Hvcn1, Tnfrsf19, |
| | Col13al, Fam78b, Gja1, Cnn2, Ppfibp2, |
| | Cldn10, Dapk2, Tmp1, Bglap3, Ramp1 |

TABLE 3
Differentially expressed genes in cluster 12 (pericytes)

| | |
|---|---|
| “known” | Acta2, Myh11, Mcam |
| Transcription | Hey1, Nr2f2, Tbx2, Ebf1, Ebf2, Foxs1, Id3, |
| Factors | Met2c, Cebpb, Zfxh3, Nr4a1, Klf9, Zeb2, |
| | Prrx1, Meox2, Junb, Id4, Zfp467, Irf1, Arid5b |
| Surface | Atp1b2, Aoc3, Sncq, Itga7, Aspn, Steap4, |
| | Thy1, Filip1I, Parm1, Agtr1a, Olfml2a, Cald1, |
| | Ednra, Col18a1, Serpini1, Bcam, Rrad, Pdgfrb, |
| | Col5a3, Pde5a |
| Secreted | Notch3, Myl1, Tinagl1, Art3, Ngf, Sparcl1, |
| | Il6, Rarres2, Vstm4, Pgf, Pdgfa, Col4a2, Igfbp7, |
| | Col4a1, Fst, Rtn4lrl1, Adamts1, Il34, Gpc6, Cscl1 |
| Other | Bgs5, Tagln, Higd1p, Nrip2, Gucv1a3, H2-M9, |
| | Des, Olfr558, Lmod1, Gucy1b3, Kcnk3, Pdlim3, |
| | Gm13861, Mrvi1, Pln, Gm13889, Ral11a, Cygp |

TABLE 4
Differentially expressed genes in
cluster 2, 10, 13, 17 (chondrocyte lineage)

| | |
|---|---|
| “known” | Sox9, Col11a2, Acan, Col2a1 |
| Transcription | Barx1, Pitx1, Foxd1, Osr2, Tbx18, Runx3, Osr2, |
| Factors | Tbx18, Runx3, Peg3, Bhlhe41, Batf3, Plagl1, |
| | Sp7, Sox8, Lef1, Shox2, Zbtb20, Foxa3, Mef2c, |
| | Egr2, Pax1, Runx2 |
| Surface | Prg4, Cpe, Mfi2, Scara3, Cpm, Chst11, Unc5q, |
| | Col11a1, Slc2a5, Slc26a2, Cspg4, Prc1, Fgfr3, |
| | Nid2, ,Spon1, Slc40a, Efemp1, Susd5, Fxyd3, |
| | Alpl, Corin, Tpd52l1, Sema3d, F5, Slc38a3 |
| Secreted | Cytl1, Rbp4, Vit, Clip, Fam19a5, Col9a3, |
| | Col9a1, Col9a2, Matn3, Hapln1, Sfrp5, Notum, |
| | Mia, lhh, Mgst2, Rarres1, Gpld1, Il17b, Bglap |
| Other | 1500015O10Rik, Itm2a, Crispld1, Meg3, Cenpp, |
| | Fxyd2, 3110079O15Rik, Lect1, Papss2, SAyt8, |
| | Stmn1, Lockd, Chil1, Calml3, Ncmap, Serpina1d, |
| | Serpina 1b, Serpina 1c, Sic6a1, Serpina1a |

TABLE 5
Differentially expressed genes in cluster 9, 15, 16 (fibroblasts)

| | |
|---|---|
| “known” | S100a4, Fn1, Col1a1, Col1a2, Lum, Col22a1, Twist2 |
| Transcription | Scx, Barx1, Trps1, Hoxd9, Pitx1, Prrx1, Rora, |
| Factors | Prrx2, Meox2, Ebf2, Osr2, Ebf1, Dlx3, |
| | Zfhx2, Meox1, Etv4, Mkx |
| Surface | Dcn, Clu, Abi3bp, Prelp, Lox, Tnxb, Col3a1, Vcan, |
| | Vi, Mfap5, Col14a1, Aspn, Pdpn, Pdgfra, F13a1, |
| | Clic5, Gpr1, Emilin2, Has1, Mtap4, Gas2, |
| | Ntng1, Serpinf1, Postn |
| Secreted | Angptl7, Clip2, Clip, Sod3, Slurp1, Spp1, Clec3b, |
| | Igfbp6, Thds4, Dpt, Gsn, Fndc1, Pla1a, Adamtsl5, |
| | Figf, Htra4, Rspo2, Mstn, Ptx4, Spock3, |
| | Cpxm2, Itgbl1 |
| Other | Anxa8, Fxyd5, Fxyd6, Egln3, Ptgis, Il33, Fgf9, Tppp3, |
| | Crlp1, Mustn1, Celf2, Tmod2, Ly6a, Fez1, Lysmd2, |
| | Pcsk6, 2210407C18Rik, Aldh1a3, Rtn1, Rab37, |
| | Lnmd, Chodl, Fam159b, Prph, Insc |

TABLE 6
Differentially expressed genes in cluster 0, 6, 11 (ECs)

| | |
|---|---|
| “known” | Kdr, Cdh5, Thbd, Emcn, Ly6e, Pecam1 Ly6a |
| Transcription | Matb, Pparg, Nr2f2, Irf8, Ets1, Sox17, Sox11, |
| Factors | Bcl6b, Gata2, Tcf15, Meox1, Sox7, Tshz2 |
| Surface | Tfpi, Gpm6a, Ackr1, Mrc1, Stab1, Vcam1, |
| | Tek, Flt1, Ramp3, Icam2, Podxl, Cd34, Mcam, |
| | Sdpr, Bcam, Tspan13, Fabp5, Vim, Kitl |
| Secreted | Lrg1, Dnase1l3, Sepp1, Egfl7, Pde2a, Gpihbp1, |
| | Sema3g, Ramp2, Cd3001g, C1qtnf9, Sparcl1, |
| | Tinagl1, Pdgfb |
| Other | Ubd, Stab2, Fabp4, Cldn5, Rgs4, Ecscr, Cyyr1, |
| | Ly6c1, Magix, Cav1, Gngt2, Myct1, Tmsb4x |

### Gene Modules

Also described herein are gene modules that are uniquely associated with the dysfunctional stromal cell subsets, including activated and repressed subsets, and key molecular nodes that control them. The present markers, marker signatures and molecular targets thus provide for new ways to evaluate and modulate stromal responses, such as to invading cancers. The gene modules described herein can be associated with a dysfunctional stromal microenvironment.

Described herein are genes and gene products differentially upregulated in stromal cell subsets, including subsets rendered dysfunctional in a hematological disease, such as leukemia, thus providing useful markers, marker signatures and molecular targets specifically for dysfunction in stromal cells.

### Stromal Cells and Cell Populations

Described herein are stromal cells and cell populations that can be characterized by a signature described elsewhere herein. The stromal cell(s) can be derived from bone marrow. In some embodiments, the stromal cell can have a signature where the signature is unique to a stromal cell type and/or state. Such signatures are described in greater detail elsewhere herein. In some embodiments, the stromal cell population can contain one or more cell types and/or states. Isolated and enriched cell populations can be generated from a mixed cell population to form isolated and enriched stromal cell populations. Isolated and/or enriched cells can be engineered and/or modulated such that they express a specific signature and/or are of a specific cell type and/or state.

In some exemplary embodiments, described herein is an isolated or engineered mesenchymal stem/stromal cell (MSC) or MSC cell population, wherein the MSC or MSC cell population is characterized by a gene signature comprised of one or more genes of Table 1. In some exemplary embodiments, the MSC or MSC cell population is characterized by a gene signature comprised of one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4. In some exemplary embodiments, the MSC or MSC cell population does not express one or more of Thy1, Ly6a (Sca-1), NG2 (Cspg4) or Nestin (Nes). In some exemplary embodiments, the gene signature comprises one or more of Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxcl1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1.

In some exemplary embodiments, described herein is an isolated or engeinered osteolineage cell (OLC) or OLC population, where the isolated or engineered OLC or OLC population is characterized by a gene signature comprising one or more genes of Table 2. In some exemplary embodiments, the OLC or OLC population is characterized by a gene signature comprising one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1. In some exemplary embodiments, the OLC or OLC population expresses Bglap and Spp1. In some exemplary embodiments, the gene signature further comprises one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmem119, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbpl11, Col3a1, Ptn, Col6a2, Tnn, Npy, Col6a1, Omd, Dcn, Tgfbi, Col6a3, or Acan. In some exemplary embodiments, the gene signature further comprises one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13.

In some exemplary embodiments, described herein is an isolated or engineered pericyte or pericyte population, wherein the isolated or engineered pericyte is characterized by a gene signature comprising one or more genes in Table 3. In some exemplary embodiments, the gene signature further comprises one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, or Cygp. In some exemplary embodiments, the gene signature further comprises one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11. In some exemplary embodiments, the gene signature further comprises one or more Acta2, Myh11, Mcam, Jag1, and Il6.

In some exemplary embodiments, described herein is an isolated or engineered chondrocyte or chondrocyte population, wherein the isolated or engineered chondrocyte population is characterized by a gene signature comprising one or more genes in Table 4. In some exemplary embodiments, the gene signature comprises one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst11, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, Il17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a. In some exemplary embodiments, the gene signature comprises one or more of Sox9, Col11a2, Acan, or Col2a1. In some exemplary embodiments, the gene signature comprises one or more of Runx2, Ihh, Mef2c, or Col10a1. In some exemplary embodiments, the gene signature further comprises one or more of Grem1, Runx2, Sp7, Alp1, or Spp1. In some exemplary embodiments, the chondrocyte expresses one or more of Ihh, Pth1r, Mef2c, Col10a1, Ibsp, Mmp13, Grem1. In some exemplary embodiments, the gene signature comprises one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, Il17b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

In some exemplary embodiments, described herein is an isolated or engineered fibroblast or fibroblast population, wherein the isolated or engineered fibroblast or fibroblast population is characterized by a gene signature comprising one or more genes of Table 5. In some exemplary embodiments, the gene signature further comprises one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgb1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc. In some exemplary embodiments, the gene signature comprises one or more of Fibronectin-1 (Fn1), Fibroblast Specific Protein-1 (S100a4), Col1a1, Col1a2, Lum, Col22a1, or Twist2. In some exemplary embodiments, the gene signature comprises one or more of Sox9, Acan, and Col2a1. In some exemplary embodiments, the gene signature comprises one or more of Cd34, Ly6a, Pdgfra, Thy1 and Cd44, and not Cdh5, or Acta2. In some exemplary embodiments, the gene signature comprises one or more of Sox-9, Scleraxis (Scx), Spp1, Cspg4, CD73 (Nt5e), and Cartilage Intermediate Layer Protein (Cilp). In some exemplary embodiments, the gene signature further comprises one or more of S1004a, Dcn, Sema3c, or Cxcl12.

In some exemplary embodiments, described herein is an isolated or engineered bone marrow derived endothelial cell (BMEC) or BMEC population, wherein the isolated or engineered fibroblast or fibroblast population is characterized by a gene signature comprising one or more genes of Table 6. In some exemplary embodiments, the gene signature comprises one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x. In some exemplary embodiments, the gene signature comprises one or more of Flt4 (Vegfr-3) and Ly6a (Sca-1), wherein Ly6a expression, when present in the gene signature, is reduced as compared to a suitable control. In some exemplary embodiments, the gene signature comprises one or more of Pecam1, Cdh5, Cd34, Tek, Lepr, Cxcl12, or Kitl. In some exemplary embodiments,

These and other aspects, objects, features, and advantages of the example embodiments will become apparent to those having ordinary skill in the art upon consideration of the following detailed description of example embodiments. the gene signature comprises one or more of Flt4, Ly6a, Icam1, or Sele. In some exemplary embodiments, the gene signature comprises one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

In some embodiments, the isolated, enriched, modulated, and/or engineered cell or cell population can be a Cluster 1, 2, 3, 4, 5, 6, 7, 8, 9, 10, 11, 12, 13, 15, 16, 17, or a subtype of any one of said Clusters as further provided and described elsewhere herein, particularly in the Working Examples herein. In some embodiments, the isolated, enriched, modulated, and/or engineered cell or cell population can have the same signature a cell of Cluster 1, 2, 3, 4, 5, 6, 7, 8, 9, 10, 11, 12, 13, 15, 16, 17, or a subtype of any one of said Clusters as further provided and described elsewhere herein, particularly in the Working Examples herein.

### Isolated and Enriched Cell Populations

Single or multiple cells can be isolated from a sample containing a mixture of cell types and/or cell states based on a signature. In some embodiments, the isolated cell population can be substantially pure. As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

In some embodiments, the isolated cell population can contain only a single cell state or cell type and can be said to be substantially free of additional cell states or cell types. As used herein, “substantially free” can mean an object species is present at non-detectable or trace levels so as not to interfere with the properties of a composition or process.

In some embodiments, isolation of stromal cells of a specific type and/or cells sate can produce an enriched population of cells that is enriched for a particular cell state and/or type. In some embodiments a cell can be enriched for a particular signature or profile. As used herein the term “enriched” can refer to increasing the amount or presence of one species in a mixed population of species relative to its amount prior to enrichment or relative to one or more other species in the mixed population. In some embodiments, an enriched population can be a substantially pure population, but such level of purity is not required to be said to be an enriched population. In some embodiments, a species in a population can be increased 1-100 fold or more in the enriched population. In some embodiments, a species in a population can be increased about 1 to 1,000 percent or more in the enriched population.

Described herein are embodiments of an isolated stromal cell and isolated stromal cell populations characterised in that the cell comprises the signature of dysfunction as defined above; to a population of said cells; to a composition or pharmaceutical composition comprising said stromal cell or said stromal cell population; and to a method for eliciting a response in a subject comprising administering to the subject said stromal cell or said stromal cell population or said pharmaceutical composition.

Described herein are isolated stromal cells that can have a specific cell identity, type and/or state. A generally applicable framework that utilizes a cell phenotype analysis technique, e.g. massively parallel single-cell RNA seq, can be used to identify cell identity, type, and/or state of an in vivo system (e.g. a stromal cell in vivo system). In vivo systems identified as having specific identity, type, and/or state can be isolated, maintained, stored, and/or used (e.g. in an ex vivo system or as a treatment that can be administered to a subject in need thereof) as desired and as described elsewhere herein. In some embodiments, the isolated cells can be used to screen for modulating agents. Methods of screening modulating agents are described elsewhere herein. In some embodiments, the specific cell state of interest to be identified can be a homeostatic cell state. In some embodiments, the specific cell state of interest to be identified can be dysfunctional or diseased cell state. In some embodiments, the specific cell type can be any one of the cell types of Clusters 1-17 as described in greater detail in the Working Examples herein. A stromal cell type, subtype, and/or a particular cell state (such as homeostatic or dysfunctional/diseased cell-state) can be identified as described elsewhere herein, such as by a unique signature. In some embodiments, the specific cell state of interest is a diseased or dysfunctional cell state, such as one that is associated with a hematological or hemopoietic disease or dysfunction.

### Methods of Preparing Isolated and Enriched Bone Marrow Stromal Cell Populations.

Isolated and enriched stromal cells and populations thereof can be generated by detecting a signature in one or more of the cells and separating them from a parent or sample population based on that signature. Signatures and methods of measuring and detecting said signatures are described in greater detail elsewhere herein. In some embodiments, the isolated or enriched cell(s) can be further cultured, expanded, manipulated, engineered, modified, and/or modulated. Such methods are described in greater detail elsewhere herein and/or will be appreciated by those of ordinary skill in the art.

In some exemplary embodiments, described herein are methods of preparing a mesenchymal stem/stromal cell (MSC) enriched cell population a stromal cell population comprising:

enriching the population of stromal cells for cells that have an MSC gene signature, wherein the gene signature comprises

a. one or more genes of Table 1;

b. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or

c. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxcl1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1;

and wherein the MCS optionally does not express one or more of Thy1, Ly6a (Sca-1), NG2 (Cspg4) or Nestin (Nes).

In some exemplary embodiments, described herein are methods of preparing an osteolineage (OLC) enriched cell population a stromal cell population comprising: enriching the population of stromal cells for cells that have an OLC gene signature, wherein the gene signature comprises

a. one or more genes of Table 2;

b. one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;

c. one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmeml19, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbp11, Col3a1, Ptn, Col6a2, Tnn, Npy, Col6a1, Omd, Dcn, Tgfbi, Col6a3, or Acan;

d. one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13;

and wherein the OLC optionally expresses Bglap and Spp1.

In some exemplary embodiments, described herein are methods of preparing a chondrocyte enriched cell population a stromal cell population comprising:

enriching the population of stromal cells for cells that have a chondrocyte gene signature, wherein the gene signature comprises

a. one or more genes of Table 4;

b. one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst1, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, Il7b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;

c. one or more of Sox9, Col11a2, Acan, or Col2a1;

d. one or more of Runx2, Ihh, Mef2c, or Col10a1;

e. one or more of Grem1, Runx2, Sp7, Alp1, or Spp1;

f. one or more of Ihh, Pth1r, Mef2c, Col10a1, Ibsp, Mmp13, Grem1; or

g. one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, Il7b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

In some exemplary embodiments, described herein are methods of preparing a fibroblast enriched cell population a stromal cell population comprising:

enriching the population of stromal cells for cells that have a fibroblast gene signature, wherein the gene signature comprises

a. one or more genes of Table 5;

b. one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;

c. Fibronectin-1 (Fn1), Fibroblast Specific Protein-1 (S100a4), Col1a1, Col1a2, Lum, Col22a1, or Twist2;

d. one or more of Sox9, Acan, and Col2a1;

e. Cd34, Ly6a, Pdgfra, Thy1 and Cd44, and not Cdh5, or Acta2;

f. one or more of Sox-9, Scleraxis (Scx), Spp1, Cspg4, CD73 (Nt5e), and Cartilage Intermediate Layer Protein (Cilp); or

g. one or more of S1004a, Dcn, Sema3c, or Cxcl12.

In some exemplary embodiments, described herein are methods of preparing a bone marrow derived endothelial cell (BMEC) enriched cell population a stromal cell population comprising:

enriching the population of stromal cells for cells that have a BMEC gene signature, wherein the gene signature comprises

a. one or more genes of Table 6;

b. one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x;

c. one or more of Flt4 (Vegfr-3) or Ly6a (Sca-1);

d. one or more of Pecam1, Cdh5, Cd34, Tek, Lepr, Cxcl12, or Kitl;

e. one or more of Flt4, Ly6a, Icam1, or Sele;

f. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, lfitm2; or

g. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

In some exemplary embodiments, described herein are methods of preparing a pericyte enriched cell population a stromal cell population comprising:

enriching the population of stromal cells for cells that have a pericyte gene signature, wherein the gene signature comprises

a. one or more genes in Table 3;

b. one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, Cygp;

c. one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11; or

d. one or more of Acta2, Myh11, Mcam, Jag1, or Il6.

In some exemplary embodiments, enriching the population of stromal cells comprises determining an MSC, an OLC, a chondrocyte, a BMEC, a fibroblast, a pericyte gene signature, or a combination thereof, wherein the gene signature(s) are determined by single cell RNA sequencing.

### Modified/Engineered Stromal Cell Populations

Described herein are modified and engineered stromal cells that can be engineered/modified to have a specific cell identity, type, and/or state. In some embodiments, cells (e.g. stromal cells) can be exposed to a modulating agent or method that is effective to modulate the identity, type, and/or state of the stromal cell prior to identification and/or isolation. Exposure of the cells to the agent can occur in vitro, ex vivo, or in vivo. In some embodiments, exposure of a stromal to the modulation agent can generate a stromal having a homeostatic cell state. In some embodiments, exposure of a stromal cell to the modulation agent can generate a stromal cell having a dysfunctional cell state. The identity, type, and/or state can be identified via an appropriate method which are described elsewhere herein, such as a method of detecting a signature in the engineered stromal cell. In some embodiments, a generally applicable framework that utilizes a cell phenotype analysis technique, e.g. massively parallel single-cell RNA seq, can be used to identify cell identity, type, and/or state of stromal cells. A homeostatic or activated cell-state in an stromal cell can be identified as described elsewhere herein. Other appropriate methods of analysis are described in greater detail elsewhere herein.

A gene, signature (e.g. a gene signature), and/or immune cell may be modified ex vivo. A gene, gene signature or immune cell may be modified in vivo. Not being bound by a theory, modifying immune and/or other cells (e.g. other stromal cells) in vivo, such that dysfunctional cells are decreased, can provide a therapeutic effect, including but not limited to enhancing an immune response and/or remodeling the bone marrow stromal cell landscape, and/or remodeling the bone marrow microenvironment in a subject. A gene, gene signature or immune cell may be modified by any suitable modulating agent. Methods of modulating cells, screening and identifying suitable modulating agents, and suitable modulating agents are described in greater detail elsewhere herein.

Methods of preparing the modified/engineered stromal cells is described in greater detail elsewhere herein.

### Cell Culture

As described elsewhere herein, a stromal cell population can include a single cell type or sub-type, a combination of cell types and/or subtypes, cell-based therapeutic, an explant, or an organoid derived using one or more of the methods disclosed herein. Such methods can include culturing the cells. Populations of cells can contain one or more cell type and/or cell state. Cells can be derived from a subject. The subject can be a human. The subject can be a non-human mammal.

In certain embodiments, the single cell type or subtype or combination of cell types and/or subtypes comprises a bone marrow stromal cell, an immune cell, intestinal cell, liver cell, kidney cell, lung cell, brain cell, epithelial cell, endoderm cell, neuron, ectoderm cell, islet cell, acinar cell, oocyte, sperm, blood cell, hematopoietic cell, hepatocyte, skin/keratinocyte, melanocyte, bone/osteocyte, hair/dermal papilla cell, cartilage/chondrocyte, fat cell/adipocyte, skeletal muscular cell, endothelium cell, cardiac muscle/cardiomyocyte, trophoblast, tumor cell, tumor microenvironment (TME) cell and combinations thereof.

In certain embodiments, the single cell type or sub-type is pluripotent, multipotent, and/or or the combination of cell types and/or subtypes comprises one or more stem cells. The one or more stem cells may be selected from the group consisting of lymphoid stem cells, mesenchymal stem cells, myeloid stem cells, neural stem cells, skeletal muscle satellite cells, epithelial stem cells, endodermal and neuroectodermal stem cells, germ cells, extraembryonic and embryonic stem cells, mesenchymal stem cells, intestinal stem cells, embryonic stem cells, and induced pluripotent stem cells (iPSCs).

As used herein, the term “stem cell” refers to a multipotent cell having the capacity to self-renew and to differentiate into multiple cell lineages.

As used herein, the term “epithelial stem cell” refers to a multipotent cell which has the potential to become committed to multiple cell lineages, including cell lineages resulting in epithelial cells.

The tumor microenvironment (TME) is the cellular environment in which the tumor exists, including surrounding blood vessels, immune cells, cancer associated fibroblasts (CAFs), bone marrow-derived inflammatory cells, lymphocytes, signaling molecules and the extracellular matrix (ECM).

Tumor infiltrating lymphocytes (TILs) are lymphocytes that penetrate a tumor.

In certain embodiments, a cell-based therapeutic includes engraftment of the cells of the present invention. As used herein, the term “engraft” or “engraftment” refers to the process of cell incorporation into a tissue of interest in vivo through contact with existing cells of the tissue.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, the term “organoid” or “epithelial organoid” refers to a cell cluster or aggregate that resembles an organ, or part of an organ, and possesses cell types relevant to that particular organ.

As used herein, a “subject” is a vertebrate, including any member of the class mammalia.

As used herein, a “mammal” refers to any mammal including but not limited to human, mouse, rat, sheep, monkey, goat, rabbit, hamster, horse, cow or pig.

A “non-human mammal”, as used herein, refers to any mammal that is not a human.

General techniques useful in the practice of this invention in cell culture and media uses are known in the art (e.g., Large Scale Mammalian Cell Culture (Hu et al. 1997. Curr Opin Biotechnol 8: 148); Serum-free Media (K. Kitano. 1991. Biotechnology 17: 73); or Large Scale Mammalian Cell Culture (Curr Opin Biotechnol 2: 375, 1991). The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture, such as temperature of 37° C., 5% v/v CO₂ and >95% humidity.

Methods related to stem cells and differentiating stem cells are known in the art (see, e.g., “Teratocarcinomas and embryonic stem cells: A practical approach” (E. J. Robertson, ed., IRL Press Ltd. 1987); “Guide to Techniques in Mouse Development” (P. M. Wasserman et al. eds., Academic Press 1993); “Embryonic Stem Cells: Methods and Protocols” (Kursad Turksen, ed., Humana Press, Totowa N.J., 2001); “Embryonic Stem Cell Differentiation in Vitro” (M. V. Wiles, Meth. Enzymol. 225: 900, 1993); “Properties and uses of Embryonic Stem Cells: Prospects for Application to Human Biology and Gene Therapy” (P. D. Rathjen et al., al., 1993). Differentiation of stem cells is reviewed, e.g., in Robertson. 1997. Meth Cell Biol 75: 173; Roach and McNeish. 2002. Methods Mol Biol 185: 1-16; and Pedersen. 1998. Reprod Fertil Dev 10: 31). For further elaboration of general techniques useful in the practice of this invention, the practitioner can refer to standard textbooks and reviews in cell biology, tissue culture, and embryology (see, e.g., Culture of Human Stem Cells (R. Ian Freshney, Glyn N. Stacey, Jonathan M. Auerbach—2007); Protocols for Neural Cell Culture (Laurie C. Doering—2009); Neural Stem Cell Assays (Navjot Kaur, Mohan C. Vemuri—2015); Working with Stem Cells (Henning Ulrich, Priscilla Davidson Negraes—2016); and Biomaterials as Stem Cell Niche (Krishnendu Roy—2010)).

Organoid technology has been previously described for example, for bone marrow, brain, retinal, stomach, lung, thyroid, small intestine, colon, liver, kidney, pancreas, prostate, mammary gland, fallopian tube, taste buds, salivary glands, and esophagus (see, e.g., Clevers, Modeling Development and Disease with Organoids, Cell. 2016 Jun. 16; 165(7):1586-1597).

For further methods of cell culture solutions and systems, see International Patent publication WO2014159356A1.

The culture methods described herein can be applied in other contexts throughout this specification as will be appreciated by those of ordinary skill in the art.

### Methods of Detecting a Bone Marrow Stromal Cell Signature

Described herein are methods of identifying genes and gene product that are differentially expressed in bone marrow stromal cells and subsets thereof. In certain embodiments, determining expression comprises detecting RNA levels. In certain embodiments, determining expression comprises detecting protein levels. Accordingly, any suitable method can be used, such as but not limited to RNA-Seq, antibodies (for example to detect surface markers) and the like.

In certain example embodiments, assessing the cell (sub)types and states present in the in sample may comprise analysis of expression matrices from the scRNA-seq expression data, performing dimensionality reduction, graph-based clustering and deriving list of cluster-specific genes in order to identify cell types and/or states present in the in vivo system. These marker genes may then be used throughout to relate one cell state to another. For example, these marker genes can be used to relate stromal cell (sub)types and/or states to the homeostatic and/or active cell (sub(types) and/or states. The same analysis may then be applied to the source material for the sample or a control. From both sets of sc-RNAseq analysis an initial distribution of gene expression data is obtained. In certain embodiments, the distribution may be a count-based metric for the number of transcripts of each gene present in a cell. Further the clustering and gene expression matrix analysis allow for the identification of key genes in the homeostatic cell-state and the stromal cell state, such as differences in the expression of key transcription factors. In certain example embodiments, this may be done conducting differential expression analysis. For example, in the Working Examples below, differential gene expression analysis identified that different stromal cell types and/or cell states have differential gene expression signatures, such as those stromal cells of Clusters 1-17 and subtypes therein and those that are dysfunctional in a diseased state. In some embodiments, the signature, program and/or module can include one or more genes as set forth in any one of Tables 1-8 and combinations thereof. The methods disclosed herein can both identify key markers of different stromal cell types and/or states and potential targets for modulation to shift the expression distribution of the stromal cells from an initial state and/or type to another. Again, turning to the Examples provided herein, the single cell transcriptomic steps of the methods disclosed herein were used to identify that the stromal cells can be of 6 broad classes, 17 types (as identified as Clusters 1-17 in the Working Examples herein) and several sub-types therein and can be present in different cell states (such as dysfunctional and normal) had differential expression of one or more genes as set forth in at least Tables 1-8 or a combination thereof. Modulation of stromal cells is discussed in greater detail elsewhere herein.

In some aspects, identification of a specific stromal cell type/subtype and/or state can include detecting a shift, such as a statistically significant shift, in the cell-state as indicated by a modulated (e.g. an increased distance) in the gene expression space between a first type/subtype and/or cell state to a second cell type/subtype and/or cell state. In some aspects the first or the second cell state is a dysfunctional or diseased cell state. In some embodiments, the dysfunction or diseased cell state is the result of bone marrow micro environment remodeling by a cancer cell or cell population. In certain embodiments, the distance is measured by a Euclidean distance, Pearson coefficient, Spearman coefficient, or combination thereof.

In certain embodiments, the gene expression space comprises 10 or more genes, 20 or more genes, 30 or more genes, 40 or more genes, 50 or more genes, 100 or more genes, 500 or more genes, or 1000 or more genes. In certain embodiments, the expression space defines one or more cell pathways. In certain embodiments, the expression space is a transcriptome of the target in vivo system.

In certain embodiments, the shift in cell type and/or cell states that increases the distance in gene expression space between homeostatic cell-state and/or dysfunctional or diseased is a statistically significant shift in the gene expression distribution of the homeostatic and/or activated cell-state toward that of the dysfunctional or diseased cell state. The statistically significant shift may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%. The statistical shift may include the overall transcriptional identity or the transcriptional identity of one or more genes, gene expression cassettes, or gene expression signatures of the dysfunctional or diseased cell state compared cell state (i.e., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% of the genes, gene expression cassettes, or gene expression signatures are statistically shifted in a gene expression distribution). A shift of 0% means that there is no difference to the homeostatic and/or activated cell state. A gene distribution may be the average or range of expression of particular genes, gene expression cassettes, or gene expression signatures in the homeostatic and/or dysfunctional or diseased cell-state (e.g., a plurality of a cell of interest from a subject may be sequenced and a distribution is determined for the expression of genes, gene expression cassettes, or gene expression signatures). In certain embodiments, the distribution is a count-based metric for the number of transcripts of each gene present in a cell. A statistical difference between the distributions indicates a shift. The one or more genes, gene expression cassettes, or gene expression signatures may be selected to compare transcriptional identity based on the one or more genes, gene expression cassettes, or gene expression signatures having the most variance as determined by methods of dimension reduction (e.g., tSNE analysis). In certain embodiments, comparing a gene expression distribution comprises comparing the initial cells with the lowest statistically significant shift as compared to the homeostatic and/or dysfunctional or diseased cell state (e.g., determining shifts when comparing only the dysfunctional or diseased cells with a shift of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10% to the homeostatic cell state). In certain example embodiments, statistical shifts may be determined by defining a homeostatic, activated, and/or diseased/dysfunctional state score.

For example, a gene list of key genes enriched in a homeostatic/activated model may be defined. To determine the fractional contribution to a cell's transcriptome to that gene list, the total log (scaled UMI+1) expression values for gene with the list of interest are summed and then divided by the total amount of scaled UMI detected in that cell giving a proportion of a cell's transcriptome dedicated to producing those genes. Thus, statistically significant shifts may be shifts in an initial score for the homeostatic score towards the dysfunctional or diseased score.

Other methods for assessing differences in the dysfunctional or diseased and homeostatic stromal cells may be employed. In certain example embodiments, an assessment of differences in the dysfunctional or diseased and homeostatic stromal cell proteome may be used to further identify key differences in cell type and sub-types or cells. states. For example, isobaric mass tag labeling and liquid chromatography mass spectroscopy may be used to determine relative protein abundances in the ex vivo and in vivo systems. Description provided elsewhere herein further disclosure on leveraging proteome analysis within the context of the methods disclosed herein.

Methods of detecting activation of a stromal cell are also described herein. In some embodiments, the method of detecting activation of a stromal cell comprising detection of a gene expression signature of activation selected from the group of:

a) a signature comprising or consisting of one or more markers selected from the group consisting of Cxcl12, Adipoq, Kit1, Lepr, Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4;

b) a signature comprising or consisting of one or more markers selected from the group consisting of Bglap, Spp1, Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;

c) a signature comprising or consisting of one or more markers selected from the group consisting of Acta2, Myh11, Mcam, Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn41r11, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, or Cygp;

d) a signature comprising or consisting of one or more markers selected from the group consisting of Sox9, Col11a2, Acan, Col2a1, Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst1, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, Il17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;

e) a signature comprising or consisting of one or more markers selected from the group consisting of S100a4, Fn1, Col1a1, Col1a2, Lum, Col22a1, Twist2, Scx, Barx1, Trps1, Hoxd9, Pitx1, Prrx1, Rora, Prrx2, Meox2, Ebf2, Osr2, Ebf1, DIx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gprl, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;

f) a signature comprising or consisting of one or more markers selected from the group consisting of Kdr, Cdh5, Thbd, Emcn, Ly6e, Pecam1 Ly6a, Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasell3, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x; or g) a signature comprising or consisting of two or more markers each independently selected from any one of the groups as defined in any one of a) to f).

In some exemplary embodiments, described herein are methods of detecting a mesenchymal stem/stromal cell (MSC) from a population of stromal cells comprising: detecting in a sample the expression or activity of a MSC gene expression signature, wherein detection of the MSC gene expression signature indicates MSCs in the sample, and wherein the MSC gene expression signature comprises:

a. one or more genes of Table 1;

b. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or

c. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxcl1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1;

and wherein the MCS optionally does not express one or more of Thy1, Ly6a (Sca-1), NG2 (Cspg4) or Nestin (Nes).

In some exemplary embodiments, described herein are methods of detecting an osteolineage cell (OLC) from a population of stromal cells comprising:

detecting in a sample the expression or activity of an OLC gene expression signature,

wherein detection of the OLC gene expression signature indicates OLCs in the sample, and

wherein the OLC gene expression signature comprises

a. one or more genes of Table 2;

b. one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;

c. one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmeml19, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbp11, Col3a1, Ptn, Col6a2, Tnn, Npy, Col6a1, Omd, Dcn, Tgfbi, Col6a3, or Acan;

d. one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13];

and wherein the OLC optionally expresses Bglap and Spp1.

In some exemplary embodiments, described herein are methods of detecting a chondrocyte from a population of stromal cells comprising:

detecting in a sample the expression or activity of a chondrocyte gene expression signature,

wherein detection of the chondrocyte gene expression signature indicates chondrocytes in the sample, and

wherein the chondrocyte gene expression signature comprises

a. one or more genes of Table 4;

b. one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst11, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, Il17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;

c. one or more of Sox9, Col11a2, Acan, or Col2a1;

d. one or more of Runx2, Ihh, Mef2c, or Col10a1;

e. one or more of Grem1, Runx2, Sp7, Alp1, or Spp1;

f. one or more of Ihh, Pth1r, Mef2c, Col10a1, Ibsp, Mmp13, Grem 1; or

g. one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, Il17b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

In some exemplary embodiments, described herein are methods of detecting a fibroblast from a population of stromal cells comprising:

detecting in a sample the expression or activity of a fibroblast gene expression signature,

wherein detection of the fibroblast gene expression signature indicates fibroblasts in the sample, and

wherein the fibroblast gene expression signature comprises

a. one or more genes of Table 5;

b. one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;

c. Fibronectin-1 (Fn1), Fibroblast Specific Protein-1 (S100a4), Col1a1, Col1a2, Lum, Col22a1, or Twist2;

d. one or more of Sox9, Acan, and Col2a1;

e. Cd34, Ly6a, Pdgfra, Thy1 and Cd44, and not Cdh5, or Acta2;

f. one or more of Sox-9, Scleraxis (Scx), Spp1, Cspg4, CD73 (Nt5e), and Cartilage Intermediate Layer Protein (Cilp); or

g. one or more of S1004a, Dcn, Sema3c, or Cxcl12.

In some exemplary embodiments, described herein are methods of detecting a bone marrow derived endothelial cell (BMEC) from a population of stromal cells comprising: detecting in a sample the expression or activity of a BMEC gene expression signature,

wherein detection of the BMEC gene expression signature indicates BMECs in the sample, and

wherein the fibroblast gene expression signature comprises

a. one or more genes of Table 6;

b. one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x;

c. one or more of Flt4 (Vegfr-3) or Ly6a (Sca-1);

d. one or more of Pecam1, Cdh5, Cd34, Tek, Lepr, Cxcl12, or Kitl;

e. one or more of Flt4, Ly6a, Icam1, or Sele;

f. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, lfitm2; or

g. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

In some exemplary embodiments, described herein are methods of detecting a pericyte from a population of stromal cells comprising:

detecting in a sample the expression or activity of a pericyte gene expression signature,

wherein detection of the pericyte gene expression signature indicates pericyte s in the sample, and

wherein the fibroblast gene expression signature comprises

a. one or more genes in Table 3;

b. one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, Cygp;

c. one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11; or

d. one or more of Acta2, Myh11, Mcam, Jag1, or Il6.

In some exemplary embodiments, the sample is obtained from the blood or bone marrow.

### Gene Expression Space and Expression Signatures

As is also discussed elsewhere herein a “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g. differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be detected by analysis of expression profiles of single-cells within a population of cells from isolated samples (e.g. tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g. cancer grade), or linked to a particular outcome or progression of the disease (e.g. metastasis), or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing different tumor cells or tumor cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

In further aspects, the invention relates to gene signatures, protein signature, and/or other genetic or epigenetic signature of particular stromal cell subpopulations, as defined herein elsewhere.

scRNA-seq may be obtained from cells using standard techniques known in the art. Some exemplary scRNA-seq techniques are discussed elsewhere herein. As discussed elsewhere herein, a collection of mRNA levels for a single cell can be called an expression profile (or expression signature) and is often represented mathematically by a vector in gene expression space. See e.g. Wagner et al., 2016. Nat. Biotechnol; 34(111): 1145-1160. This is a vector space that has a dimension corresponding to each gene, with the value of the ith coordinate of an expression profile vector representing the number of copies of mRNA for the ith gene. Note that real cells only occupy an integer lattice in gene expression space (because the number of copies of mRNA is an integer), but it is assumed herein that cells can move continuously through a real-valued G dimensional vector space.

As an individual cell changes the genes it expresses over time, it moves in gene expression space and describes a trajectory. As a population of cells develops and grows, a distribution on gene expression space evolves over time. When a single cell from such a population is measured with single cell RNA sequencing, a noisy estimate of the number of molecules of mRNA for each gene is obtained. The measured expression profile of this single cell is represented as a sample from a probability distribution on gene expression space. This sampling captures both (a) the randomness in the single cell RNA sequencing measurement process (due to sub-sampling reads, technical issues, etc.) and (b) the random selection of a cell from a population. This probability distribution is treated as nonparametric in the sense that it is not specified by any finite list of parameters.

A precise mathematical notion for a developmental process as a generalization of a stochastic process is provided below. A goal of the methods disclosed herein is to infer the ancestors and descendants of subpopulations evolving according to an unknown developmental, disease, and/or other physiological process and/or corresponding to a specific cell state at the beginning, end, or any point during the developmental process. While not bound by a particular theory, this may be possible over short time scales because it is reasonable to assume that cells don't change too much and therefore it can be inferred which cells go where. It will be appreciated that “developmental” when used in this context is not limited to the “growth/maturity” of an organism/cell, but rather refers to any characteristic that can change temporally and/or spatially such that the characteristic can be said to “develop” over time and/or space through a “developmental process”.

In certain example embodiments, the following definitions to define a precise notion of the developmental trajectory of an individual cell and its descendants are used. It is a continuous path in gene expression that bifurcates with every cell division. Formally, consider a cell x(o)∈ [symbol] ^{G}. Let k(t)≥0 specify the number of descendants at time t, where k(0)=1. A single cell developmental trajectory is a continuous function

$$x \text{:} [ 0 , T ) \to \underbrace{{G}^{} \times {G}^{} \times … \times {G}^{}}_{k ( t ) times} .$$

This means that x(t) is a k(t)-tuple of cells, each represented by a vector [symbol] ^{G}:

x(t)=(x₁(t), . . . ,xₖ₍ₜ₎(t)).

Cells x₁(t), . . . , xₖ₍ₜ₎(t) as the descendants of x(o).

[symbol] ^{G} and R^{G} are used interchangeably.

Note that the temporal dynamics of an individual cell cannot be directly measured because scRNA-Seq is a destructive measurement process: scRNA-Seq lyses cells so it is only possible to measure the expression profile of a cell at a single point in time. As a result, it is not possible to directly measure the descendants of that cell, and it is (usually) not possible to directly measure which cells share a common ancestor with ordinary scRNA-Seq. Therefore, the full trajectory of a specific cell is unobservable. However, one can learn something about the probable trajectories of individual cells by measuring snapshots from an evolving population.

Published methods typically represent the aggregate trajectory of a population of cells with a graph. While this recapitulates the branching path traveled by the descendants of an individual cell, it may over-simplify the stochastic nature of developmental processes. Individual cells have the potential to travel through different paths, but in reality any given cell travels one and only one such path. The methods disclosed herein help to describe this potential, which might not be a represented by a graph as a union of one-dimensional paths.

Instead, a developmental process is defined to be a time-varying distribution on gene expression space. The word distribution is used to refer to an object that assigns mass to regions of [symbol] _{G}. Note that a distinction is made between distribution and probability distribution, which necessarily has total mass 1. Distributions are formally defined as generalized functions (such as the delta function δ_{X}) that act on test functions. A used herein, a “distribution” is the same as a measure. One simple example of a distribution of cells is that a set of cells x₁, . . . , xₙ can be represented by the distribution

$$= {\sum }_{i = 1}^{n} {δ}_{{x}_{i}} .$$

Similarly, a set of single cell trajectories may be represented x₁(t), . . . , xₙ(t) with a distribution over trajectories. A developmental process [symbol] ₜ is a time-varying distribution on gene expression space. A developmental process generalizes the definition of stochastic process. A developmental process with total mass 1 for all time is a (continuous time) stochastic process, i.e. an ordered set of random variables with a particular dependence structure. Recall that a stochastic process is determined by its temporal dependence structure, i.e. the coupling between random variables at different time points. The coupling of a pair of random variables refers to the structure of their joint distribution. The notion of coupling for developmental processes is the same as for stochastic processes, except with general distributions replacing probability distributions.

A coupling of a pair of distributions P, Q on R^{G} is a distribution π on R^{G}×R^{G} with the property that π has P and Q as its two marginals. A coupling is also called a transport map.

As a distribution on the product space R^{G}×R^{G}, a transport map π assigns a number π(A, B) to any pair of sets A, B ⊂R^{G}.

π(A,B)=∫_{x∈A}∫_{y∈B}π(x,y)dxdy.

When π is the coupling of a developmental process, this number π(A, B) represents the mass transported from A to B by the developmental or other process. This is the amount of mass coming from A and going to B. When a particular destination is note specified, the quantity π(A, ⋅) specifies the full distribution of mass coming from A. This action may be referred to as pushing A through the transport map π. More generally, we can also push a distribution μ forward through the transport map π via integration

μ [symbol] ∫π(x,⋅)dμ(x).

The reverse operation is referred to as pulling a set B back through π. The resulting distribution π(⋅, B) encodes the mass ending up at B. Distributions can also be pulled back through π in a similar way:

μ [symbol] ∫π(⋅,y)dμ(y).

This may also be referred as back-propagating the distribution μ (and to pushing μ forward as forward propagation).

Recall that a stochastic process is Markov if the future is independent of the past, given the present. Equivalently, it is fully specified by its couplings between pairs of time points. A general stochastic process can be specified by further higher order couplings. Markov developmental processes, which are defined in the same way:

A Markov developmental process Pₜ is a time-varying distribution on R^{G} that is completely specified by couplings between pairs of time points. It is an interesting question to what extent developmental processes are Markov. On gene expression space, they are likely not Markov because, for example, the history of gene expression can influence chromatin modifications, which may not themselves be reflected in the observed expression profile but could still influence the subsequent evolution of the process. However, it is possible that developmental processes could be considered Markov on some augmented space.

A definition of descendants and ancestors of subgroups of cells evolving according to a Markov developmental process is now provided. The earlier definition of descendants is extended as follows: Consider a set of cells S⊂R^{G} which live at time t₁ are part of a population of cells evolving according to a Markov developmental process Pₜ. Let π denote the transport map for Pₜ from time t₁ to time t₂. The descendants of S at time t₂ are obtained by pushing S through the transport map π. Note that if a developmental process is not Markov, then the descendants of S are not well defined. The descendants would depend on the cells that gave rise to S, which we refer to as the ancestors of S.

Definition 6 (ancestors in a Markov developmental process). Consider a set of cells S ⊂R^{G} which live at time t₂ and are part of a population of cells evolving according to a Markov developmental process Pₜ. Let π denote the transport map for Pₜ from time t₂ to time t₁. The ancestors of S at time t₁ are obtained by pushing S through the transport map π.

### Empirical Developmental Processes

In certain aspects, a goal of the embodiments disclosed herein is to track the evolution of a developmental process from a scRNA-Seq time course. Suppose we are given input data consisting of a sequence of sets of single cell expression profiles, collected at T different time slices of development. Mathematically, this time series of expression profiles is a sequence of sets S₁, . . . , S_{T} ⊂R^{G} collected at times t₁, . . . , t_{T} ∈R.

Developmental time series. A developmental time series is a sequence of samples from a developmental process Pₜ on R^{G}. This is a sequence of sets S₁, . . . , S_{N} ⊂R^{G}. Each Sᵢ is a set of expression profiles in R^{G} drawn i.i.d from the probability distribution obtained by normalizing the distribution Pₜᵢ tohavetotalmass1. From this input data, we form an empirical version of the developmental process. Specifically, at each time point tᵢ we form the empirical probability distribution supported on the data x∈Sᵢ is formed. This is summarized inin the following definition:

Empirical developmental process. An empirical developmental process [symbol] is a time vary-ing distribution constructed from a developmental time course S₁, . . . , S_{N}:

$${{t}_{i}}_{} = \frac{1}{\vert {S}_{i} \vert} {\sum }_{x ∈ {S}_{i}} {δ}_{x} .$$

he empirical developmental process is undefined for t ∈/{t₁, . . . , t_{N}}.

Our goal is to recover information about a true, unknown developmental process Pₜ from the empirical developmental process [symbol] . The measurement process of single cell RNA-Seq destroys the coupling, and the observed empirical developmental process does not come with an informative coupling between successive time points. Over short time scales, it is reasonable to assume that cells do not change too much and therefore inferences regarding which cells go where and estimate the coupling.

This may be done with optimal transport: the transport map it that minimizes the total work required for redistributing [symbol] to [symbol] is selected. One motivation for minimizing this objective, is a deep relationship between optimal transport and dynamical systems that provides a direct connection to Waddington's landscape: the optimal transport problem can formulated as a least-action advection of one distribution into another according to an unknown velocity field (see Theorem 1 in Section 6 below). At a high level, differentiation follows a velocity field on gene expression space, and the potential inducing this velocity field is in direct correspondence with Waddington's landscape¹.

Optimal Transport for scRNA-Seq Time Series

A process for how to compute probabilistic flows from a time series of single cell gene expression profiles by using optimal transport (S1) is provided. The embodiments disclosed herein show how to compute an optimal coupling of adjacent time points by solving a convex optimization problem.

Optimal transport defines a metric between probability distributions; it measures the total distance that mass must be transported to transform one distribution into another. For two measures P and Q on R^{G}, a transport plan is a measure on the product space R^{G}×R^{G} that has marginals P and Q. In probability theory, this is also called a coupling. Intuitively, a transport plan πcan be interpreted as follows: if one picks a point mass at position x, then π(x, ⋅) gives the distribution over points where x might end up.

If c(x, y) denotes the cost² of transporting a unit mass from x to y, then the expected cost under a transport plan π is given by

∫∫c(x,y)π(x,y)dxdy.

The optimal transport plan minimizes the expected cost subject to marginal constraints:

$$\begin{matrix}{minimize}_{π} & \int \int c ( x , y ) π ( x , y ) dxdy \\ subject to & \int π ( x , • ) dx = \\ & \int π ( • , y ) dy = .\end{matrix}$$

Note that this is a linear program in the variable it because the objective and constraints are both linear in π. Note that the optimal objective value defines the transport distance between P and Q (it is also called the Earthmover's distance or Wasserstein distance). Unlike most other ways to compare distributions (such as KL-divergence or total variation), optimal transport takes the geometry of the underlying space into account. For example, the KL-Divergence is infinite for any two distributions with disjoint support, but the transport distance between two unit masses depends on their separation.

When the measures P and Q are supported on finite subsets of R^{G}, the transport plan is a matrix whose entries give transport probabilities and the linear program above is finite dimensional. In this context, empirical distributions are formed from the sets of samples S₁, . . . , S_{T}:

$${{t}_{i}}_{} = \frac{1}{\vert {S}_{i} \vert} {\sum }_{x ∈ {S}_{i}} {δ}_{x} ,$$

were δ_{X} denotes the Dirac delta function centered at x ∈R^{G}. These empirical distributions [symbol] are definitely supported, and so it is possible solve the linear program[1] with P= [symbol] and Q=

[symbol]

However, the classical formulation [1] does not allow cells to grow (or die) during transportation (because it was designed to move piles of dirt and conserve mass). When the classical formulation is applied to a time series with two distinct subpopulations proliferating at different rates³, the transport map will artificially transport mass between the subpopulations to account for the relative proliferation. Therefore, we modify the classical formulation of optimal transport in equation [1] is modified to allow cells to grow at different rates.

Is it assumed that a cell's measured expression profile x determines its growth rate g(x). This is reasonable because many genes are involved in cell proliferation (e.g. cell cycle genes). It is further assumed g(x) is a known function (based on knowledge of gene expression) representing the exponential increase in mass per unit time, but also note that the growth rate can be allowed to be miss-specified by leveraging techniques from unbalanced transport (S2). In practice, g(x) is defined in terms of the expression levels of genes involved in cell proliferation.

Derivation of Transport with Growth

For any cell x ∈Sᵢ₋₁, let r(x, y) be the fraction of x that transitions towards y. Then the amount of probability mass from x that ends up at y (after proliferation) is

r(x,y)g(x)^{Δt},

where Δₜ=tᵢ₊₁−tᵢ. The total amount of mass that comes from x can be written two ways:

$${\sum }_{y ∈ {S}_{i + 1}} r ( x , y ) {g ( x )}^{Δ t} \approx {g ( x )}^{Δ t} d {{t}_{i}}_{} ( x ) .$$

This gives us a first constraint. Similarly, there is also the constraint that the total mass observed at y is equal to the sum of masses coming from each x and ending up at y. In symbols,

$$d {{t}_{i + 1}}_{} ( y ) {\sum }_{x ∈ {S}_{i}} {g ( x )}^{Δ t} \approx {\sum }_{x ∈ {S}_{i}} r ( x , y ) {g ( x )}^{Δ t} for each y ∈ {S}_{i + 1} .$$

The factor x∈Sᵢ g(x)^{Δt} on the left hand side accounts for the overall proliferation of all the cells from Sᵢ. Note that this factor is required so that the constraints are consistent: when one sums up both sides of the first constraint over x, this must equal the result of summing up both sides of the second constraint over y. Finally, for convenience these constraints are rewritten in terms of the optimization variable

π(x,y)=r(x,y)g(x)^{Δ}^{ₜ}.

Therefore, to compute the transport map between the empirical distributions of expression profiles observed at time tᵢ and tᵢ₊₁, the following linear program is set up:

$$\begin{matrix}{minimize}_{π} & {\sum }_{x ∈ {S}_{i}} {\sum }_{y ∈ {S}_{i + 1}} c ( x , y ) π ( x , y ) \\ subject to & {\sum }_{x ∈ {S}_{i}} π ( x , y ) \approx d {{t}_{i + 1}}_{} ( y ) {\sum }_{x ∈ {S}_{i}} {g ( x )}^{Δ t} \\ & {\sum }_{y ∈ {S}_{i + 1}} π ( x , y ) \approx d {{t}_{i}}_{} ( x ) {g ( x )}^{Δ t}\end{matrix}$$

### Regularization and Algorithmic Considerations

Fast algorithms have been recently developed to solve an entropically regularized version of the transport linear program (S3). Entropic regularization means adding the entropy H(π)=E_{π} log π to the objective function, which penalizes deterministic transport plans (a purely deterministic transport plan would have only one nonzero entry in each row). Entropic regularization speeds up the computations because it makes the optimization problem strongly convex, and gradient ascent on the dual can be realized by successive diagonal matrix scalings (S3). These are very fast operations. This scaling algorithm has also been extended to work in the setting of unbalanced transport, where equality constraints are relaxed to bounds on KL-divergence (S2). This allows the growth rate function g(x) to be misspecified to some extent.

Both entropic regularization and unbalanced transport may be used. To compute the transport map between the empirical distributions of expression profiles observed at time tᵢ and tᵢ₊₁, the embodiments disclosed herein solve the following optimization problem:

$$\begin{matrix}{minimize}_{π} & {\sum }_{x ∈ {S}_{i}} {\sum }_{y ∈ {S}_{i + 1}} c ( x , y ) π ( x , y ) - ϵℋ ( π ) \\ subject to & KL [ {\sum }_{x ∈ {S}_{i}} π ( x , y ) ‖ d {{t}_{i + 1}}_{} ( y ) {\sum }_{x ∈ {S}_{i}} {g ( x )}^{Δ t} ] \leq \frac{1}{{λ}_{1}} \\ & KL [ {\sum }_{y ∈ {S}_{i + 1}} π ( x , y ) ‖ d {{t}_{i}}_{} ( x ) {g ( x )}^{Δ t} ] \leq \frac{1}{{λ}_{2}}\end{matrix}$$

where ε, λ₁ and λ₂ are regularization parameters. This is a convex optimization problem in the matrix variable π∈R^{N}^{ᵢ}^{×N}^{ᵢ₊₁}, where Nᵢ=|Sᵢ| is the number of cells sequenced at time tᵢ. It takes about 5 seconds to solve this unbalanced transport problem using the scaling algorithm of Chizat et al. 2016 (S2) on a standard laptop with Nᵢ≈5000. Note that the densities (on the discrete set Sᵢ) of the empirical distributions specified in equation [2] are simply d [symbol] (x)=1. However, in principle one could use nonuniform empirical distributions (e.g. i Nᵢ if one wanted to include information about cell quality).

To summarize: given a sequence of expression profiles S₁, . . . , S_{T}, the optimization problem [5] for each successive pair of time points Sᵢ, Sᵢ₊₁ is solved. This gives us a sequence of transport maps.

To make this more precise, consider a single cell y∈Sᵢ. The column π(⋅, y) of the transport map π from tᵢ₋₁ to tᵢ describes the contributions to y of the cells in Sᵢ₋₁. This is the origin of y at the time point tᵢ₋₁. Similarly, the row r(y, ⋅) of the transition map from tᵢ to tᵢ₊₁ describes the probabilities y would transition to cells in Sᵢ₊₁. These are the fates of y, i.e. the descendants of y.

The origin of y further back in time may be computed via matrix multiplication: the contributions to y of cells in Sᵢ₋₂ are given by a column of the matrix

\tilde{π}_{[i-2,i]}=π[_{i-2,i-1]}π_{[i-1,i]}.

This matrix [symbol] represents the inferred transport from time point tᵢ₋₂ to tᵢ, and note it with a tilde to distinguish it from the maps computed directly from adjacent time points. Note that, in principle, the transport between any non-consecutive pairs of time points Sᵢ, Sⱼ, may be directly computed but it is not anticipated that the principle of optimal transport to be as reliable over long time gaps.

Finally, note that expression profiles can be interpolated between pairs of time points by averaging a cell's expression profile at time tᵢ with its fated expression profiles at time tᵢ₊₁.

### Transport Maps Encode Regulatory Information

Transport maps can encode regulatory information, and provided herein are methods on how to set up a regression to fit a regulatory function to our sequence of transport maps. It is assumed that a cell's trajectory is cell-autonomous and, in fact, depends only on its own internal gene expression. This is wrong as it ignores paracrine signaling between cells, and we return to discuss models that include cell-cell communication at the end of this section. However, this assumption is powerful because it exposes the time-dependence of the stochastic process Pₜ as arising from pushing an initial measure through a differential equation:

\dot{x}=ƒ(x).

Here f is a vector field that prescribes the flow of a particle x. The biological motivation for estimating such a function f is that it encodes information about the regulatory networks that create the equations of motion in gene-expression space.

It is proposed to set up a regression to learn a regulatory function f that models the fate of a cell at time tᵢ₊₁ as a function of its expression profile at time tᵢ. For motivation that the transport maps might contain information about the underlying regulatory dynamics, we appeal to a classical theorem establishing a dynamical formulation of optimal transport.

Theorem 1 (Benamou and Brenier, 2001). The optimal objective value of the transport problem [1] is equal to the optimal objective value of the following optimization problem.

$$\begin{matrix}{minimize}_{ρ , v} & {\int }_{0}^{⊥} {‖ v ( t , x ) ‖}^{2} ρ ( t , x ) dtdx \\ subject to & ρ ( 0 , • ) = , ρ ( 1 , • ) = \\ & ∇ \cdot ( ρ v ) = \frac{\partial ρ}{\partial t}\end{matrix} .$$

In this theorem, v is a vector-valued velocity field that advects4 the distribution p from P to Q, and the objective value to be minimized is the kinetic energy of the flow (mass×squared velocity). Intuitively, the theorem shows that a transport map π can be seen as a point-to-point summary of a least-action continuous time flow, according to an unknown velocity field. While the optimization problem [8] can be reformulated as a convex optimization problem, and modified to allow for variable growth rates, it is inherently infinite dimensional and therefore difficult to solve numerically.

It is therefore proposed a tractable approach to learn a static regulatory function f from our sequence of transport maps. This approach involves sampling pairs of points using the couplings from optimal transport, and solving a regression to learn a regulatory function that predicts the fate of a cell at time tᵢ₊₁ as a function of its expression profile at time tᵢ:

### Regulatory Network Regression

For each pair of time points tᵢ, tᵢ₊₁, we consider the pair of random variables Xₜ, Xₜ jointly distributed according to r_{[t, t]}, (which we obtained from the i i+1 i i+1 transport map π[tᵢ, tᵢ₊₁] by removing the effect of proliferation as in equation [3]). We set up the following optimization problem over regulatory functions f:

$${min}_{f ∈ ℱ} {𝔼}_{r} {‖ \frac{{X}_{{t}_{i}} - {X}_{{t}_{i + 1}}}{{Δ}_{t}} - f ( {X}_{{t}_{i}} ) ‖}^{2} .$$

Here F specifies a parametric function class to optimize over.

### Cell Non-Autonomous Processes

This section discusses an approach to cell-cell communication. Note that the gradient flow [8] only makes sense for cell autonomous processes. Otherwise, the rate of change in expression x⋅ is not just a function of a cell's own expression vector x(t), but also of other expression vectors from other cells. We can accommodate cell non-autonomous processes by allowing f to also depend on the full distribution Pₜ

$$\frac{dx}{dt} = f ( x , {t}_{} ) .$$

### Extensions to Continuous Time.

In this section it is discussed how this method could be improved by going beyond pairs of time points to track the continuous evolution of Pₜ. It is begun by pointing out a peculiar behavior of the method: whenever we have a time point with few sampled cells, our method is forced through an information bottleneck. As an extreme example—suppose there is a time point with only one cell. Everything would transition through that single cell, which is absurd! In this extreme case, we would be better off ignoring the time point. It is therefore proposed a smoothed approach that shares information between time slices and gracefully improves as data is added.

The continuous-time formulation is based on locally-weighted averaging, an elementary interpolation technique. Recall that given noisy function evaluations yᵢ≈f(xᵢ), one can interpolate f by averaging the yᵢ for all xᵢ close to a point of interest x:

$$f ( x ) \approx {\sum }_{i} {α}_{i} f ( {x}_{i} ) ,$$

where aᵢ are weights that give more influence to nearby points

In this setup, it is sought to interpolate a distribution-valued function Pₜ from the collections of i.i.d. samples S₁, . . . , S_{T}. We can interpolate a distribution-valued function by computing the barycenter (or centroid) of nearby time points with respect to the optimal transport metric. The transport barycenter of

$${\sum }_{i = 1}^{T} {α}_{i} {W}^{2} ( {i}_{} , ) ,$$

where W (P, Q) denotes the transport distance (or Wasserstein distance) between P and Q. The transport distance is defined by the optimal value of the transport problem [1]. The weights αᵢ can be chosen to interpolate about time point t by setting, for example,

$${\sum }_{i = 1}^{T} {α}_{i} {G}^{2} ( {{t}_{i}}_{} , ) ,$$

where G(P, Q) denotes our modified transport distance from equation [5]. To solve this optimization problem, we can fix the support of Q to the samples observed at all time points ∪Tᵢ₌₁Sᵢ. Then we can apply the scaling algorithm for unbalanced bary centers due to Chizat et a1.

However, fixing the support of the barycenter ahead of time may not be completely satisfactory, and this motivates further research in the computation of transport bary centers: can we design an algorithm to solve for the barycenter Q without fixing the support in advance? Is there a dynamic formulation for bary centers analogous to the Brenier Benamou formula of Theorem 1, and can be leveraged to better learn gene regulatory networks?

Finally, this section is concluded with the observation that this continuous-time approach could pro-vide a principled approach to sequential experimental design. Optimal time points can be identified for further data collection by examining the loss function (fit of barycenter) across time, and adding data where the fit is poor. Moreover, this continuous time approach can also be used to test the principle of optimal transport by withholding some time points and testing the quality of the interpolation against the held-out truth.

### Nucleic Acid Barcode, Barcode, and Unique Molecular Identifier (UMI)

The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA such that multiple species can be sequenced together.

Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell.

In preferred embodiments, sequencing is performed using unique molecular identifiers (UMI). The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR or multiple displacement amplification (MDA).

In certain embodiments, an UMI with a random sequence of between 4 and 20 base pairs is added to a template, which is amplified and sequenced. In preferred embodiments, the UMI is added to the 5′ end of the template. Sequencing allows for high resolution reads, enabling accurate detection of true variants. As used herein, a “true variant” will be present in every amplified product originating from the original clone as identified by aligning all products with a UMI. Each clone amplified will have a different random UMI that will indicate that the amplified product originated from that clone. Background caused by the fidelity of the amplification process can be eliminated because true variants will be present in all amplified products and background representing random error will only be present in single amplification products (See e.g., Islam S. et al., 2014. Nature Methods No:11, 163-166). Not being bound by a theory, the UMI's are designed such that assignment to the original can take place despite up to 4-7 errors during amplification or sequencing. Not being bound by a theory, an UMI may be used to discriminate between true barcode sequences.

Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associate with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type NNNNNNNNNNNN.

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing based methods. For example, variable length probes or primers can be used to distinguish barcodes (for example, origin-specific barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

### Barcodes Reversibly Coupled to Solid Substrate

In some embodiments, the origin-specific barcodes are reversibly coupled to a solid or semisolid substrate. In some embodiments, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules.

Barcode with Cleavage Sites

A nucleic acid barcode may be cleavable from a specific binding agent, for example, after the specific binding agent has bound to a target molecule. In some embodiments, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, at least one cleavage site is oriented such that the cleavage at the site releases the origin-specific barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage.

### Barcode Adapters

In some embodiments, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

Barcode with Capture Moiety

In some embodiments, an origin-specific barcode further includes a capture moiety, covalently or non-covalently linked. Thus, in some embodiments the origin-specific barcode, and anything bound or attached thereto, that include a capture moiety are captured with a specific binding agent that specifically binds the capture moiety. In some embodiments, the capture moiety is adsorbed or otherwise captured on a surface. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include: incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments, the specific binding agent has been immobilized for example on a solid support, thereby isolating the origin-specific barcode.

### Other Barcoding Embodiments

DNA barcoding is also a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102(23):8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

A desirable locus for DNA barcoding can be standardized so that large databases of sequences for that locus can be developed. Most of the taxa of interest have loci that are sequencable without species-specific PCR primers. CBOL Plant Working Group, “A DNA barcode for land plants” PNAS 106(31): 12794-12797 (2009). Further, these putative barcode loci are believed short enough to be easily sequenced with current technology. Kress et al., “DNA barcodes: Genes, genomics, and bioinformatics” PNAS 105(8):2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105(8):2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31): 12569 (2009).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94).

Unique Molecular Identifiers are short (usually 4-10 bp) random barcodes added to transcripts during reverse-transcription. They enable sequencing reads to be assigned to individual transcript molecules and thus the removal of amplification noise and biases from RNA-seq data. Since the number of unique barcodes (4N, N—length of UMI) is much smaller than the total number of molecules per cell (˜106), each barcode will typically be assigned to multiple transcripts. Hence, to identify unique molecules both barcode and mapping location (transcript) must be used. UMI-sequencing typically consists of paired-end reads where one read from each pair captures the cell and UMI barcodes while the other read consists of exonic sequence from the transcript. UMI-sequencing typically consists of paired-end reads where one read from each pair captures the cell and UMI barcodes while the other read consists of exonic sequence from the transcript.

In some embodiments, the nucleic acids of the library are flanked by switching mechanism at 5′ end of RNA templates (SMART). SMART is a technology that allows the efficient incorporation of known sequences at both ends of cDNA during first strand synthesis, without adaptor ligation. The presence of these known sequences is crucial for a number of downstream applications including amplification, RACE, and library construction. While a wide variety of technologies can be employed to take advantage of these known sequences, the simplicity and efficiency of the single-step SMART process permits unparalleled sensitivity and ensures that full-length cDNA is generated and amplified. (see, e.g., Zhu et al., 2001, Biotechniques. 30 (4): 892-7.

After processing the reads from a UMI experiment, the following conventions are often used: 1. The UMI is added to the read name of the other paired read. 2. Reads are sorted into separate files by cell barcode ° For extremely large, shallow datasets, a cell barcode may be added to the read name as well to reduce the number of files. A cell barcode indicates the cell from which mRNA is captured (e.g., Drop-Seq or Seq-Well).

### Sequencing Methods.

In one approach, the present invention relates to a PCR-amplification based approach to derive genetic information from single-cell RNA-seq libraries.

The method generally involves two PCR steps and size selection. Initially, a library is constructed wherein each sequence comprises a SMART sequence at the 5′ end and the 3′ end, a genetic region of interest at the 5′ end and a UMI and Cell BC at the 3′ end, e.g., 5′ SMART-genetic region of interest-UMI-Cell BC-SMART 3′.

A first PCR product is generated by amplifying sequences with a biotinylated 5′ primer comprising a binding site for a second PCR product and a sequence complementary to a specific gene of interest and a 3′ SMART primer complementary to the SMART sequence at the 3′ end of the nucleic acid to generate a first PCR product. The binding site for the second PCR product may be a partial Illumina sequencing primer binding site or an oligomer for sequencing kit, such as a NEBNext® oligos for Illumina® sequencing (see, e.g., https://www.neb.com/applications/library-preparation-for-next-generati n-sequencing/illumina-library-preparation/products).

The 5′ primer comprising the binding site for the second PCR product to amplify the first PCR product may further comprise a sequence to bind a flow cell, a sequence allowing multiple sequencing libraries to be sequenced simultaneously and/or a sequence providing an additional primer binding site. The sequence to bind a flow cell may be a P7 sequence and the flow cell may be an Illumina® flowcell.

In another embodiment, the SMART primer complementary to the SMART sequence at the 3′ end of the nucleic acid to amplify the first PCR product may further comprise a sequence to allow fragments to bind a flowcell. The sequence to allow fragments to bind a flowcell may be a P5 sequence.

Regardless of the library construction method, submitted libraries may consist of a sequence of interest flanked on either side by adapter constructs. On each end, these adapter constructs may have flow cell binding sites, P5 and P7, which allow the library fragment to attach to the flow cell surface. The P5 and P7 regions of single-stranded library fragments anneal to their complementary oligos on the flowcell surface. The flow cell oligos act as primers and a strand complementary to the library fragment is synthesized. The original strand is washed away, leaving behind fragment copies that are covalently bonded to the flowcell surface in a mixture of orientations. 1,000 copies of each fragment are generated by bridge amplification, creating clusters. For simplification, the diagram shows only one copy (out of 1,000) in each cluster, and only two clusters (out of 30-50 million). The P5 region is cleaved, resulting in clusters containing only fragments which are attached by the P7 region. This ensures that all copies are sequenced in the same direction. The sequencing primer anneals to the P5 end of the fragment, and begins the sequencing by synthesis process. Index reads are only performed when a sample is barcoded. When Read 1 is finished, everything from Read 1 is removed and an index primer is added, which anneals at the P7 end of the fragment and sequences the barcode. Everything is stripped from the template, which forms clusters by bridge amplification as in Read 1. This leaves behind fragment copies that are covalently bonded to the flowcell surface in a mixture of orientations. This time, P7 is cut instead of P5, resulting in clusters containing only fragments which are attached by the P5 region. This ensures that all copies are sequences in the same direction (opposite Read 1). The sequencing primer anneals to the P7 region and sequences the other end of the template.

In another embodiment, the sequence allowing multiple sequencing libraries to be sequenced simultaneously may be an INDEX sequence. The INDEX allows multiple sequencing libraries to be sequenced simultaneously (and demultiplexed using Illumina's bcl2fastq command). See, e.g., https://support.illumina.com/downloads/illumina-customer-sequence-letter.html for exemplary INDEX sequences.

In another embodiment, the 5′ primer comprising the binding site for the second PCR product to amplify the first PCR product may further comprise a NEXTERA sequence. See, e.g., https://support.illumina.com/downloads/illumina-customer-sequence-letter.html and U.S. Pat. Nos. 5,965,443, and 6,437,109 and European Patent No. 0927258, for exemplary NEXTERA sequences.

In another embodiment, the sequence providing an additional primer binding site may be a custom readl primer binding site (CR1P) for sequencing. CR1P is a Custom Readl Primer binding site that is used for Drop-Seq and Seq-Well library sequencing. CR1P may comprise the sequence: GCCTGTCCGCGGAAGCAGTGGTATCAACGCAGAGTAC (SEQ ID NO: 1) (see e.g., Gierahn et al., Nature Methods 14, 395-398 (2017).

Biotin-NEXT-GENE-for: Biotinylation enables purification of the desired product following the first PCR reaction. NEXT creates a binding site for the second PCR product as well as a partial primer binding site for standard Illumina sequencing kits. NEXT may be any sequence that allows targeted enrichment and then select addition of sequencing handles. GENE is a sequence complementary to the WTA, designed to amplify a specific region of interest (usually an exon).

SMART-rev: The SMART sequence is used in Drop-seq and Seq-Well to generate WTA libraries. Because the polyT-unique molecular identifier-unique cellular barcode (polyT-UMI-CB) sequence is followed by the SMART sequence, and the template switching oligo (TSO) also contains the SMART sequence, WTA libraries have the SMART sequence as a PCR binding site on both the 5′ and the 3′ end.

P7-INDEX-NEXTERA: The P7 sequence allows fragments to bind the Illumina flowcell. The INDEX allows multiple sequencing libraries to be sequenced simultaneously (and demultiplexed using Illumina's bcl2fastq command). The NEXTERA sequence provides a primer binding site for Illumina's standard Read2 sequencing primer mix.

SMART-CR1P-P5: The SMART sequence is the same as in SMART-rev. CRIP is a Custom Read1 Primer binding site that is used for Drop-Seq and Seq-Well library sequencing. The P5 sequence allows fragments to bind the Illumina flowcell. Note that the primer design can be easily modified for compatibility with additional single-cell RNA-seq technologies (SMART) or sequencing technologies (NEXTERA, CRIP).

The method also provides for biotin enrichment of the first PCR product. Biotinylation of the primer to amplify the gene, region or mutation of interest from the library allows for the purification of the PCR product of interest. Because the libraries are flanked with SMART sequences on both ends, the vast majority of the first PCR product would be amplification of the entire library. Without the biotinylated primer, enrichment of the gene, region or mutation of interest would be insufficient to efficiently and confidently call genetic mutations. Biotin enrichment may be accomplished by streptavidin binding of the biotinylated first PCR product. The streptavidin bead kilobaseBINDER kit (Thermo Fisher Cat #60101) allows for isolation of large biotinylated DNA fragments.

Gene specific primers may be mixed for simultaneous detection of multiple mutations. Libraries may also be mixed for simultaneous detection of mutations in multiple samples. However, mixed primers sometimes may not detect multiple mutations in the same gene as only the shortest fragment will be detected.

The present method may be adapted to identify any gene, region or mutation of interest and to identify cells containing specific genes, regions or mutations, deletions, insertions, indels, or translocations of interest.

A gene or groups of genes of interest may be, for example, one or more genes that are part of or make up a homeostatic stromal cell gene expression signature, a dysfunctional stromal cell gene expression signature, or a combination thereof. The gene or groups of genes of interest may be, for example, a hematological disease-related gene of interest. Hematological diseases of interest are described in greater detail else where herein.

### Sequencing and Library Construction

In some embodiments, RNA-seq can be used. As used herein, RNA-seq methods refer to high-throughput single-cell RNA-sequencing protocols. RNA-seq includes, but is not limited to, Drop-seq, Seq-Well, InDrop and 1Cell Bio. RNA-seq methods also include, but are not limited to, smart-seq2, TruSeq, CEL-Seq, STRT, ChIRP-Seq, GRO-Seq, CLIP-Seq, Quartz-Seq, or any other similar method known in the art (see, e.g., “Sequencing Methods Review” Illumina® Technology, https://www.illumina.com/content/dam/illumina-marketing/documents/products/research_reviews/sequencing-methods-review.pdf. See e.g., Wagner et al., 2016. Nat Biotechnol. 34(111): 1145-1160.

In some embodiments, sequence adapters can be used. As used herein, sequence adapters or sequencing adapters or adapters include primers that may include additional sequences involved in for example, but not limited to, flowcell binding, cluster generation, library generation, sequencing primers, sequences for Seq-Well, and/or custom read sequencing primers. Universal primer recognition sequences

The present invention may encompass incorporation of SMART sequences into the library. Switching mechanism at 5′ end of RNA template (SMART) is a technology that allows the efficient incorporation of known sequences at both ends of cDNA during first strand synthesis, without adaptor ligation. The presence of these known sequences is crucial for a number of downstream applications including amplification, RACE, and library construction. While a wide variety of technologies can be employed to take advantage of these known sequences, the simplicity and efficiency of the single-step SMART process permits unparalleled sensitivity and ensures that full-length cDNA is generated and amplified. (see, e.g., Zhu et al., 2001, Biotechniques. 30 (4): 892-7.

A pooled set of nucleic acids that are tagged refer to a plurality of nucleic acid molecules that results from incorporating an identifiable sequence tag into a pool of sample-tagged nucleic acids, by any of various methods. In some embodiments, the tag serves instead as a minimal sequence adapter for adding nucleic acids onto sample-tagged nucleic acids, rendering the pool compatible with a particular DNA sequencing platform or amplification strategy.

In some embodiments, a 3′ barcoded single cell RNA library can be generated. The 3′ barcoded single cell RNA library includes a plurality of nucleic acids, each nucleic acid including a gene of interest, a unique molecular identifier (UMI) and a cell barcode (cell BC). The cell barcode is located on the 3′ end of the transcript. As the single cell RNA library comprises a cell barcode on the 3′ end of the transcripts, at least a subset of the library from the 3′ barcoded single cell RNA library contains a transcript of interest at least 1 kb away from the 3′ end of the transcript. The 5′ side of transcripts are typically underrepresented in standard 3′ barcoded libraries.

In a preferred embodiment, each nucleic acid sequence is flanked by switching mechanism at 5′ end of RNA template (SMART) sequences at the 5′ end and 3′ end, that is, in this embodiment, an exemplary nucleic acid in the library would be 5′ SMART-genetic region of interest-UMI-Cell BC-SMART 3′.

Multiple technologies have been described that massively parallelize the generation of single cell RNA seq libraries that can be used in the present disclosure. As used herein, RNA-seq methods refer to high-throughput single-cell RNA-sequencing protocols. RNA-seq includes, but is not limited to, Drop-seq, Seq-Well, InDrop and 1Cell Bio. RNA-seq methods also include, but are not limited to, smart-seq2, TruSeq, CEL-Seq, STRT, ChIRP-Seq, GRO-Seq, CLIP-Seq, Quartz-Seq, or any other similar method known in the art (see, e.g., “Sequencing Methods Review” Illumina® Technology, Sequencing Methods Review available at illumina.com.

In certain embodiments, the invention involves plate based single cell RNA sequencing (see, e.g., Picelli, S. et al., 2014, “Full-length RNA-seq from single cells using Smart-seq2” Nature protocols 9, 171-181, doi: 10. 1038/nprot.2014.006).

In some embodiments, Drop-sequence methods or Drop-seq are contemplated for the present invention and can be used. Cells come in different types, sub-types and activity states, which are classify based on their shape, location, function, or molecular profiles, such as the set of RNAs that they express. RNA profiling is in principle particularly informative, as cells express thousands of different RNAs. Approaches that measure for example the level of every type of RNA have until recently been applied to “homogenized” samples—in which the contents of all the cells are mixed together. Methods to profile the RNA content of tens and hundreds of thousands of individual human cells have been recently developed, including from brain tissues, quickly and inexpensively. To do so, special microfluidic devices have been developed to encapsulate each cell in an individual drop, associate the RNA of each cell with a ‘cell barcode’ unique to that cell/drop, measure the expression level of each RNA with sequencing, and then use the cell barcodes to determine which cell each RNA molecule came from. See, e.g., methods of Macosko et al., 2015, Cell 161, 1202-1214 and Klein et al., 2015, Cell 161, 1187-1201 are contemplated for the present invention.

In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. Furthermore, incorporation of microfluidics technology enhances system integration and automation. Microfluidic reactions are generally conducted in microdroplets or microwells. The ability to conduct reactions in microdroplets depends on being able to merge different sample fluids and different microdroplets. See, e.g., US Patent Publication No. 20120219947. See also international patent application serial no. PCT/US2014/058637 for disclosure regarding a microfluidic laboratory on a chip.

Droplet/microwell microfluidics offers significant advantages for performing high-throughput screens and sensitive assays. Droplets allow sample volumes to be significantly reduced, leading to concomitant reductions in cost. Manipulation and measurement at kilohertz speeds enable up to 108 discrete biological entities (including, but not limited to, individual cells or organelles) to be screened in a single day. Compartmentalization in droplets increases assay sensitivity by increasing the effective concentration of rare species and decreasing the time required to reach detection thresholds. Droplet microfluidics combines these powerful features to enable currently inaccessible high-throughput screening applications, including single-cell and single-molecule assays. See, e.g., Guo et al., Lab Chip, 2012, 12, 2146-2155.

Drop-Sequence methods and apparatus provides a high-throughput single-cell RNA-Seq and/or targeted nucleic acid profiling (for example, sequencing, quantitative reverse transcription polymerase chain reaction, and the like) where the RNAs from different cells are tagged individually, allowing a single library to be created while retaining the cell identity of each read. A combination of molecular barcoding and emulsion-based microfluidics to isolate, lyse, barcode, and prepare nucleic acids from individual cells in high-throughput is used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode ˜10,000-100,000 cells.

InDrop™, also known as in-drop seq, involves a high-throughput droplet-microfluidic approach for barcoding the RNA from thousands of individual cells for subsequent analysis by next-generation sequencing (see, e.g., Klein et al., Cell 161(5), pp 1187-1201, 21 May 2015). Specifically, in in-drop seq, one may use a high diversity library of barcoded primers to uniquely tag all DNA that originated from the same single cell. Alternatively, one may perform all steps in drop.

Well-based biological analysis or Seq-Well is also contemplated for the present invention. The well-based biological analysis platform, also referred to as Seq-well, facilitates the creation of barcoded single-cell sequencing libraries from thousands of single cells using a device that contains 100,000 40-micron wells. Importantly, single beads can be loaded into each microwell with a low frequency of duplicates due to size exclusion (average bead diameter 35.μm). By using a microwell array, loading efficiency is greatly increased compared to drop-seq, which requires poisson loading of beads to avoid duplication at the expense of increased cell input requirements. Seq-well, however, is capable of capturing nearly 100% of cells applied to the surface of the device.

Seq-well is a methodology which allows attachment of a porous membrane to a container in conditions which are benign to living cells. Combined with arrays of picoliter-scale volume containers made, for example, in PDMS, the platform provides the creation of hundreds of thousands of isolated dialysis chambers which can be used for many different applications. The platform also provides single cell lysis procedures for single cell RNA-seq, whole genome amplification or proteome capture; highly multiplexed single cell nucleic acid preparation (about 100× increase over current approaches); highly parallel growth of clonal bacterial populations thus providing synthetic biology applications as well as basic recombinant protein expression; selection of bacterial that have increased secretion of a recombinant product possible product could also be small molecule metabolite which could have considerable utility in chemical industry and biofuels; retention of cells during multiple microengraving events; long term capture of secreted products from single cells; and screening of cellular events. Principles of the present methodology allow for addition and subtraction of materials from the containers, which has not previously been available on the present scale in other modalities.

Seq-Well also enables stable attachment (through multiple established chemistries) of porous membranes to PDMS nanowell devices in conditions that do not affect cells. Based on requirements for downstream assays, amines are functionalized to the PDMS device and oxidized to the membrane with plasma. With regard to general cell culture uses, the PDMS is amine functionalized by air plasma treatment followed by submersion in an aqueous solution of poly(lysine) followed by baking at 80° C. For processes that require robust denaturing conditions, the amine must be covalently linked to the surface. This is accomplished by treating the PDMS with air plasma, followed by submersion in an ethanol solution of amine-silane, followed by baking at 80° C., followed by submersion in 0.2% phenylene diisothiocyanate (PDITC) DMF/pyridine solution, followed by baking, followed by submersion in chitosan or poly(lysine) solution. For functionalization of the membrane for protein capture, membrane can be amine-silanized using vapor deposition and then treated in solution with NHS-biotin or NHS-maleimide to turn the amine groups into the crosslinking species.

After functionalization, the device is loaded with cells (bacterial, mammalian or yeast) in compatible buffers. The cell-laden device is then brought in contact with the functionalized membrane using a clamping device. A plain glass slide is placed on top of the membrane in the clamp to provide force for bringing the two surfaces together. After an hour incubation, as one hour is a preferred time span, the clamp is opened and the glass slide is removed. The device can then be submerged in any aqueous buffer for days without the membrane detaching, enabling repetitive measurements of the cells without any cell loss. The covalently-linked membrane is stable in many harsh buffers including guanidine hydrochloride which can be used to robustly lyse cells. If the pore size of the membrane is small, the products from the lysed cells will be retained in each well. The lysing buffer can be washed out and replaced with a different buffer which allows binding of biomolecules to probes preloaded in the wells. The membrane can then be removed, enabling addition of enzymes to reverse transcribe or amplify nucleic acids captured in the wells after lysis. Importantly, the chemistry enables removal of one membrane and replacement with a membrane with a different pore size to enable integration of multiple activities on the same array.

As discussed, while the platform has been optimized for the generation of individually barcoded single-cell sequencing libraries following confinement of cells and mRNA capture beads (Macosko, et al. Cell. 2015 May 21; 161(5): 1202-1214), it is capable of multiple levels of data acquisition. The platform is compatible with other assays and measurements performed with the same array. For example, profiling of human antibody responses by integrated single-cell analysis is discussed with regard to measuring levels of cell surface proteins (Ogunniyi, A. O., B. A. Thomas, T. J. Politano, N. Varadarajan, E. Landais, P. Poignard, B. D. Walker, D. S. Kwon, and J. C. Love, “Profiling Human Antibody Responses by Integrated Single-Cell Analysis” Vaccine, 32(24), 2866-2873.) The authors demonstrate a complete characterization of the antigen-specific B cells induced during infections or following vaccination, which enables and informs one of skill in the art how interventions shape protective humoral responses. Specifically, this disclosure combines single-cell profiling with on-chip image cytometry, microengraving, and single-cell RT-PCR.

The invention provides a method for creating a single-cell sequencing library comprising: merging one uniquely barcoded mRNA capture microbead with a single-cell in an emulsion droplet having a diameter of 75-125 μm; lysing the cell to make its RNA accessible for capturing by hybridization onto RNA capture microbead; performing a reverse transcription either inside or outside the emulsion droplet to convert the cell's mRNA to a first strand cDNA that is covalently linked to the mRNA capture microbead; pooling the cDNA-attached microbeads from all cells; and preparing and sequencing a single composite RNA-Seq library.

The invention provides a method for preparing uniquely barcoded mRNA capture microbeads, which has a unique barcode and diameter suitable for microfluidic devices comprising: 1) performing reverse phosphoramidite synthesis on the surface of the bead in a pool-and-split fashion, such that in each cycle of synthesis the beads are split into four reactions with one of the four canonical nucleotides (T, C, G, or A) or unique oligonucleotides of length two or more bases; 2) repeating this process a large number of times, at least two, and optimally more than twelve, such that, in the latter, there are more than 16 million unique barcodes on the surface of each bead in the pool. (See http://www.ncbi.nlm.nih.gov/pmc/articles/PMC206447).

In another embodiment, the invention encompasses making beads specific to the panel of desired mutations or mutations plus mRNA and a capture of both. In one embodiment, one or more mutation hot spots may be near the 3′ end.

Generally, the invention provides a method for preparing a large number of beads, particles, microbeads, nanoparticles, or the like with unique nucleic acid barcodes comprising performing polynucleotide synthesis on the surface of the beads in a pool-and-split fashion such that in each cycle of synthesis the beads are split into subsets that are subjected to different chemical reactions; and then repeating this split-pool process in two or more cycles, to produce a combinatorially large number of distinct nucleic acid barcodes. Invention further provides performing a polynucleotide synthesis wherein the synthesis may be any type of synthesis known to one of skill in the art for “building” polynucleotide sequences in a step-wise fashion. Examples include, but are not limited to, reverse direction synthesis with phosphoramidite chemistry or forward direction synthesis with phosphoramidite chemistry. Previous and well-known methods synthesize the oligonucleotides separately then “glue” the entire desired sequence onto the bead enzymatically. Applicants present a complexed bead and a novel process for producing these beads where nucleotides are chemically built onto the bead material in a high-throughput manner. Moreover, Applicants generally describe delivering a “packet” of beads which allows one to deliver millions of sequences into separate compartments and then screen all at once.

The invention further provides an apparatus for creating a single-cell sequencing library via a microfluidic system, comprising: an oil-surfactant inlet comprising a filter and a carrier fluid channel, wherein said carrier fluid channel further comprises a resistor; an inlet for an analyte comprising a filter and a carrier fluid channel, wherein said carrier fluid channel further comprises a resistor; an inlet for mRNA capture microbeads and lysis reagent comprising a filter and a carrier fluid channel, wherein said carrier fluid channel further comprises a resistor; said carrier fluid channels have a carrier fluid flowing therein at an adjustable or predetermined flow rate; wherein each said carrier fluid channels merge at a junction; and said junction being connected to a mixer, which contains an outlet for drops.

A mixture comprising a plurality of microbeads adorned with combinations of the following elements: bead-specific oligonucleotide barcodes created by the discussed methods; additional oligonucleotide barcode sequences which vary among the oligonucleotides on an individual bead and can therefore be used to differentiate or help identify those individual oligonucleotide molecules; additional oligonucleotide sequences that create substrates for downstream molecular-biological reactions, such as oligo-dT (for reverse transcription of mature mRNAs), specific sequences (for capturing specific portions of the transcriptome, or priming for DNA polymerases and similar enzymes), or random sequences (for priming throughout the transcriptome or genome). In an embodiment, the individual oligonucleotide molecules on the surface of any individual microbead contain all three of these elements, and the third element includes both oligo-dT and a primer sequence.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 1251, 3H, and 1311), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added.

Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

The fluorescent label may be a fluorescent protein, such as blue fluorescent protein, cyan fluorescent protein, green fluorescent protein, red fluorescent protein, yellow fluorescent protein or any photoconvertible protein. Colormetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code.

In an advantageous embodiment, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation.

The invention discussed herein enables high throughput and high-resolution delivery of reagents to individual emulsion droplets that may contain cells, organelles, nucleic acids, proteins, etc. through the use of monodisperse aqueous droplets that are generated by a microfluidic device as a water-in-oil emulsion. The droplets are carried in a flowing oil phase and stabilized by a surfactant. In one aspect single cells or single organelles or single molecules (proteins, RNA, DNA) are encapsulated into uniform droplets from an aqueous solution/dispersion. In a related aspect, multiple cells or multiple molecules may take the place of single cells or single molecules. The aqueous droplets of volume ranging from 1 pL to 10 nL work as individual reactors. Disclosed embodiments provide 10⁴ to 10⁵ single cells in droplets which can be processed and analyzed in a single run.

To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination.

Each species of droplet is introduced at a confluence point in a main microfluidic channel from separate inlet microfluidic channels. Preferably, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length is selected such that faster species of droplets catch up to the slowest species. Size constraints of the channel prevent the faster moving droplets from passing the slower moving droplets resulting in a train of droplets entering a merge zone. Multi-step chemical reactions, biochemical reactions, or assay detection chemistries often require a fixed reaction time before species of different type are added to a reaction. Multi-step reactions are achieved by repeating the process multiple times with a second, third or more confluence points each with a separate merge point. Highly efficient and precise reactions and analysis of reactions are achieved when the frequencies of droplets from the inlet channels are matched to an optimized ratio and the volumes of the species are matched to provide optimized reaction conditions in the combined droplets.

Fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc. In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be affected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. In another embodiment, the expansion and/or contraction of the liquid reservoir may be combined with other flow-controlling devices and methods, e.g., as discussed herein. Non-limiting examples of devices able to cause the expansion and/or contraction of a liquid reservoir include pistons.

Key elements for using microfluidic channels to process droplets include: (1) producing droplet of the correct volume, (2) producing droplets at the correct frequency and (3) bringing together a first stream of sample droplets with a second stream of sample droplets in such a way that the frequency of the first stream of sample droplets matches the frequency of the second stream of sample droplets. Preferably, bringing together a stream of sample droplets with a stream of premade library droplets in such a way that the frequency of the library droplets matches the frequency of the sample droplets.

Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions, e.g., a library may contain plurality of different compounds at a range of concentrations encapsulated as separate library elements for screening their effect on cells or enzymes, alternatively a library could be composed of a plurality of different primer pairs encapsulated as different library elements for targeted amplification of a collection of loci, alternatively a library could contain a plurality of different antibody species encapsulated as different library elements to perform a plurality of binding assays. The introduction of a library of reaction conditions onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second. However, in practice droplets pack with oil between them that slowly drains. Over time the carrier fluid drains from the library droplets and the number density of the droplets (number/mL) increases. Hence, a simple fixed rate of infusion for the drive fluid does not provide a uniform rate of introduction of the droplets into the microfluidic channel in the substrate. Moreover, library-to-library variations in the mean library droplet volume result in a shift in the frequency of droplet introduction at the confluence point. Thus, the lack of uniformity of droplets that results from sample variation and oil drainage provides another problem to be solved. For example, if the nominal droplet volume is expected to be 10 pico-liters in the library, but varies from 9 to 11 pico-liters from library-to-library then a 10,000 pico-liter/second infusion rate will nominally produce a range in frequencies from 900 to 1,100 droplet per second. In short, sample to sample variation in the composition of dispersed phase for droplets made on chip, a tendency for the number density of library droplets to increase over time and library-to-library variations in mean droplet volume severely limit the extent to which frequencies of droplets may be reliably matched at a confluence by simply using fixed infusion rates. In addition, these limitations also have an impact on the extent to which volumes may be reproducibly combined. Combined with typical variations in pump flow rate precision and variations in channel dimensions, systems are severely limited without a means to compensate on a run-to-run basis. The foregoing facts not only illustrate a problem to be solved, but also demonstrate a need for a method of instantaneous regulation of microfluidic control over microdroplets within a microfluidic channel.

Combinations of surfactant(s) and oils must be developed to facilitate generation, storage, and manipulation of droplets to maintain the unique chemical/biochemical/biological environment within each droplet of a diverse library. Therefore, the surfactant and oil combination must (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition to the requirements on the droplet library function and stability, the surfactant-in-oil solution must be coupled with the fluid physics and materials associated with the platform. Specifically, the oil solution must not swell, dissolve, or degrade the materials used to construct the microfluidic chip, and the physical properties of the oil (e.g., viscosity, boiling point, etc.) must be suited for the flow and operating conditions of the platform.

Droplets formed in oil without surfactant are not stable to permit coalescence, so surfactants must be dissolved in the oil that is used as the continuous phase for the emulsion library. Surfactant molecules are amphiphilic—part of the molecule is oil soluble, and part of the molecule is water soluble. When a water-oil interface is formed at the nozzle of a microfluidic chip for example in the inlet module discussed herein, surfactant molecules that are dissolved in the oil phase adsorb to the interface. The hydrophilic portion of the molecule resides inside the droplet and the fluorophilic portion of the molecule decorates the exterior of the droplet. The surface tension of a droplet is reduced when the interface is populated with surfactant, so the stability of an emulsion is improved. In addition to stabilizing the droplets against coalescence, the surfactant should be inert to the contents of each droplet and the surfactant should not promote transport of encapsulated components to the oil or other droplets.

A droplet library may be made up of a number of library elements that are pooled together in a single collection (see, e.g., US Patent Publication No. 2010002241). Libraries may vary in complexity from a single library element to 1015 library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” are also referred to herein as an “emulsion library” or “emulsion libraries.” These terms are used interchangeably throughout the specification.

A cell library element may include, but is not limited to, hybridomas, B-cells, primary cells, cultured cell lines, cancer cells, stem cells, cells obtained from tissue, or any other cell type. Cellular library elements are prepared by encapsulating a number of cells from one to hundreds of thousands in individual droplets. The number of cells encapsulated is usually given by Poisson statistics from the number density of cells and volume of the droplet. However, in some cases the number deviates from Poisson statistics as discussed in Edd et al., “Controlled encapsulation of single-cells into monodisperse picolitre drops.” Lab Chip, 8(8): 1262-1264, 2008. The discrete nature of cells allows for libraries to be prepared in mass with a plurality of cellular variants all present in a single starting media and then that media is broken up into individual droplet capsules that contain at most one cell. These individual droplets capsules are then combined or pooled to form a library consisting of unique library elements. Cell division subsequent to, or in some embodiments following, encapsulation produces a clonal library element.

A bead-based library element may contain one or more beads, of a given type and may also contain other reagents, such as antibodies, enzymes or other proteins. In the case where all library elements contain different types of beads, but the same surrounding media, the library elements may all be prepared from a single starting fluid or have a variety of starting fluids. In the case of cellular libraries prepared in mass from a collection of variants, such as genomically modified, yeast or bacteria cells, the library elements will be prepared from a variety of starting fluids.

Often it is desirable to have exactly one cell per droplet with only a few droplets containing more than one cell when starting with a plurality of cells or yeast or bacteria, engineered to produce variants on a protein. In some cases, variations from Poisson statistics may be achieved to provide an enhanced loading of droplets such that there are more droplets with exactly one cell per droplet and few exceptions of empty droplets or droplets containing more than one cell.

Examples of droplet libraries are collections of droplets that have different contents, ranging from beads, cells, small molecules, DNA, primers, antibodies. Smaller droplets may be in the order of femtoliter (fL) volume drops, which are especially contemplated with the droplet dispensors. The volume may range from about 5 to about 600 fL. The larger droplets range in size from roughly 0.5 micron to 500 micron in diameter, which corresponds to about 1 pico liter to 1 nano liter. However, droplets may be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges.

The droplets comprised within the emulsion libraries of the present invention may be contained within an immiscible oil which may comprise at least one fluorosurfactant. In some embodiments, the fluorosurfactant comprised within immiscible fluorocarbon oil is a block copolymer consisting of one or more perfluorinated polyether (PFPE) blocks and one or more polyethylene glycol (PEG) blocks. In other embodiments, the fluorosurfactant is a triblock copolymer consisting of a PEG center block covalently bound to two PFPE blocks by amide linking groups. The presence of the fluorosurfactant (similar to uniform size of the droplets in the library) is critical to maintain the stability and integrity of the droplets and is also essential for the subsequent use of the droplets within the library for the various biological and chemical assays discussed herein. Fluids (e.g., aqueous fluids, immiscible oils, etc.) and other surfactants that may be utilized in the droplet libraries of the present invention are discussed in greater detail herein.

The present invention provides an emulsion library which may comprise a plurality of aqueous droplets within an immiscible oil (e.g., fluorocarbon oil) which may comprise at least one fluorosurfactant, wherein each droplet is uniform in size and may comprise the same aqueous fluid and may comprise a different library element. The present invention also provides a method for forming the emulsion library which may comprise providing a single aqueous fluid which may comprise different library elements, encapsulating each library element into an aqueous droplet within an immiscible fluorocarbon oil which may comprise at least one fluorosurfactant, wherein each droplet is uniform in size and may comprise the same aqueous fluid and may comprise a different library element, and pooling the aqueous droplets within an immiscible fluorocarbon oil which may comprise at least one fluorosurfactant, thereby forming an emulsion library.

For example, in one type of emulsion library, all different types of elements (e.g., cells or beads), may be pooled in a single source contained in the same medium. After the initial pooling, the cells or beads are then encapsulated in droplets to generate a library of droplets wherein each droplet with a different type of bead or cell is a different library element. The dilution of the initial solution enables the encapsulation process. In some embodiments, the droplets formed will either contain a single cell or bead or will not contain anything, i.e., be empty. In other embodiments, the droplets formed will contain multiple copies of a library element. The cells or beads being encapsulated are generally variants on the same type of cell or bead. In one example, the cells may comprise cancer cells of a tissue biopsy, and each cell type is encapsulated to be screened for genomic data or against different drug therapies. Another example is that 1011 or 1015 different type of bacteria; each having a different plasmid spliced therein, are encapsulated. One example is a bacterial library where each library element grows into a clonal population that secretes a variant on an enzyme.

In another example, the emulsion library may comprise a plurality of aqueous droplets within an immiscible fluorocarbon oil, wherein a single molecule may be encapsulated, such that there is a single molecule contained within a droplet for every 20-60 droplets produced (e.g., 20, 25, 30, 35, 40, 45, 50, 55, 60 droplets, or any integer in between). Single molecules may be encapsulated by diluting the solution containing the molecules to such a low concentration that the encapsulation of single molecules is enabled. In one specific example, a LacZ plasmid DNA was encapsulated at a concentration of 20 fM after two hours of incubation such that there was about one gene in 40 droplets, where 10 m droplets were made at 10 kHz per second. Formation of these libraries rely on limiting dilutions.

Methods of the invention involve forming sample droplets. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety.

In certain embodiments, the carrier fluid may contain one or more additives, such as agents which reduce surface tensions (surfactants). Surfactants can include Tween, Span, fluorosurfactants, and other agents that are soluble in oil relative to water. In some applications, performance is improved by adding a second surfactant to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing.

In certain embodiments, the droplets may be surrounded by a surfactant which stabilizes the droplets by reducing the surface tension at the aqueous oil interface. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglyceryl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

By incorporating a plurality of unique tags into the additional droplets and joining the tags to a solid support designed to be specific to the primary droplet, the conditions that the primary droplet is exposed to may be encoded and recorded. For example, nucleic acid tags can be sequentially ligated to create a sequence reflecting conditions and order of same. Alternatively, the tags can be added independently appended to solid support. Non-limiting examples of a dynamic labeling system that may be used to bioninformatically record information can be found at US Provisional Patent Application entitled “Compositions and Methods for Unique Labeling of Agents” filed Sep. 21, 2012 and Nov. 29, 2012. In this way, two or more droplets may be exposed to a variety of different conditions, where each time a droplet is exposed to a condition, a nucleic acid encoding the condition is added to the droplet each ligated together or to a unique solid support associated with the droplet such that, even if the droplets with different histories are later combined, the conditions of each of the droplets are remain available through the different nucleic acids. Non-limiting examples of methods to evaluate response to exposure to a plurality of conditions can be found at US Provisional Patent Application entitled “Systems and Methods for Droplet Tagging” filed Sep. 21, 2012.

Applications of the disclosed device may include use for the dynamic generation of molecular barcodes (e.g., DNA oligonucleotides, fluorophores, etc.) either independent from or in concert with the controlled delivery of various compounds of interest (drugs, small molecules, siRNA, CRISPR guide RNAs, reagents, etc.). For example, unique molecular barcodes can be created in one array of nozzles while individual compounds or combinations of compounds can be generated by another nozzle array. Barcodes/compounds of interest can then be merged with cell-containing droplets. An electronic record in the form of a computer log file is kept to associate the barcode delivered with the downstream reagent(s) delivered. This methodology makes it possible to efficiently screen a large population of cells for applications such as single-cell drug screening, controlled perturbation of regulatory pathways, etc. The device and techniques of the disclosed invention facilitate efforts to perform studies that require data resolution at the single cell (or single molecule) level and in a cost-effective manner. Disclosed embodiments provide a high throughput and high-resolution delivery of reagents to individual emulsion droplets that may contain cells, nucleic acids, proteins, etc. through the use of monodisperse aqueous droplets that are generated one by one in a microfluidic chip as a water-in-oil emulsion. Hence, the invention proves advantageous over prior art systems by being able to dynamically track individual cells and droplet treatments/combinations during life cycle experiments. Additional advantages of the disclosed invention provide an ability to create a library of emulsion droplets on demand with the further capability of manipulating the droplets through the disclosed process(es). Disclosed embodiments may, thereby, provide dynamic tracking of the droplets and create a history of droplet deployment and application in a single cell-based environment.

Droplet generation and deployment is produced via a dynamic indexing strategy and in a controlled fashion in accordance with disclosed embodiments of the present invention. Disclosed embodiments of the microfluidic device discussed herein provides the capability of microdroplets that be processed, analyzed and sorted at a highly efficient rate of several thousand droplets per second, providing a powerful platform which allows rapid screening of millions of distinct compounds, biological probes, proteins or cells either in cellular models of biological mechanisms of disease, or in biochemical, or pharmacological assays.

The term “tagmentation” refers to a step in the Assay for Transposase Accessible Chromatin using sequencing (ATAC-seq) as described. (See, Buenrostro, J. D., Giresi, P. G., Zaba, L. C., Chang, H. Y., Greenleaf, W. J., Transposition of native chromatin for fast and sensitive epigenomic profiling of open chromatin, DNA-binding proteins and nucleosome position. Nature methods 2013; 10 (12): 1213-1218). Specifically, a hyperactive Tn5 transposase loaded in vitro with adapters for high-throughput DNA sequencing, can simultaneously fragment and tag a genome with sequencing adapters. In one embodiment the adapters are compatible with the methods described herein.

In certain embodiments, tagmentation is used to introduce adaptor sequences to genomic DNA in regions of accessible chromatin (e.g., between individual nucleosomes) (see, e.g., US20160208323A1; US20160060691A1; WO2017156336A1; and Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aabl601. Epub 2015 May 7). In certain embodiments, tagmentation is applied to bulk samples or to single cells in discrete volumes.

The 3′ barcoded libraries can be used in the methods as described herein to provide enriched libraries containing transcripts of interest that are not as abundant or accessible in the original single cell RNAseq libraries. Other Seq-Well embodiments that may be used with the current invention are described in PCT Application entitled “Functionalized Solid Support” filed on Oct. 23, 2018, Attorney Docket No. BROD-2840WP.

### Transcripts of Interest

A transcript of interest may also be referred to interchangeably as a gene of interest or target sequence. Target sequence can refer to any polynucleotide, such as DNA or RNA polynucleotides. In some embodiments, a target sequence is derived from the nucleus or cytoplasm of a cell, and may include nucleic acids in or from mitochondrial, organelles, vesicles, liposomes or particles present within the cell and subjected to a single cell sequencing method, retaining identification of the source cell or subcellular organelle.

A gene of interest may comprise, for example, a mutation, deletion, insertion, translocation, single nucleotide polymorphism (SNP), splice variant or any combination thereof associated with a particular attribute in a gene of interest. In another embodiment, the gene of interest may be a cancer gene. In another embodiment, the gene of interest is a mutated cancer gene, such as a somatic mutation.

Any gene, region or mutation of interest and to identify cells containing specific genes, regions or mutations, deletions, insertions, indels, or translocations of interest can be included in the libraries. A gene of interest may be, for example, a hematological disease gene, such as a blood cancer gene, an/or a stromal cell state and/or type/subtype gene. Such a gene can have a mutation. In some embodiments, the stromal cell state or type associated gene can be one or more specific to a homeostatic or non-diseased cell state.

In some instances, the mutation is located anywhere in the gene. In some instances, the desired transcript can be greater than about 1 kb away from the cell barcode of the nucleic acid of the libraries as described here. The gene of interest may comprise a SNP.

As the methods herein can be designed to distinguish SNPs within a population, the methods may be used to distinguish pathogenic strains that differ by a single SNP or detect certain disease specific SNPs, such as but not limited to, disease associated SNPs, such as without limitation cancer associated SNPs.

The gene of interest, transcript of interest, in some instances comprises a mutation.

Mutation within 1 kilobase of the polyA tail of an mRNA in the library.

In some instances, the library can include a transcript of interest, or desired transcript is in a T cell or a B cell. In some instances, the transcript of interest is in a T cell receptor, a B cell receptor or a CAR-T cell. In some instances, the transcript of interest is in variable regions of a sequence, all variable regions of, for example a T cell receptor c/p.

The transcript of interest can be derived from a cell. In some embodiments a T cell, or a B cell. In some embodiments a TCR, A BCR, or a CAR-T cell. In some instances, the methods target variable regions of a transcript of interest. In some instances, the gene of interest is in a cancer cell. In some instances, it is a blood cancer cell. In some instances, it is a leukemia cell, sucha as an AML celll. In some instances, the cell can be characterized by the highly expressed genes comprised with in a cell, and may be characterized as a GMP like cell, HSC/progenitor like cell or a myeloid cell.

In another embodiment, the specific gene of interest may be a tumor protein P53 gene. Specific mutations include, but are not limited to, positions P152R and/or Q144P in the tumor protein P53 gene.

In some aspects, there is no mutation but regulation changes as a result of a diseased/dysfunctional state and/or remodeling of a bone marrow microenvironment that can be present as a result of a disease agent or cell, which then can result in a change of gene expression by the stromal cell and a shift in cell state or type.

In some embodiments, the transcript of interest is one corresponding to a gene as in any of Tables 1-8.

### Methods of Distinguishing Cells by Genotype

In an embodiment, the present invention relates to a method of distinguishing cells by genotype by enriching libraries for transcripts of interest which may comprise a PCR-based method, for example: constructing a library comprising a plurality of nucleic acids wherein each nucleic acid may comprise a gene, a unique molecular identifier (UMI) and a cell barcode (cell BC) flanked by switching mechanism at 5′ end of RNA template (SMART) sequences at the 5′ and 3′ end, amplifying each nucleic acid in the library to create a first PCR product using a tagged 5′ primer which may comprise a binding site for a second PCR product and a sequence complementary to a specific gene of interest and a 3′ SMART primer complementary to the SMART sequence at the 3′ end of the nucleic acid thereby generating a first PCR product, selective enrichment of the first PCR product by binding to the tag introduced by the 5′ primer or a targeted 3′ capture with a bifunctional bead or targeted capture bead, amplifying the tag-enriched first PCR product with a 5′ primer which may comprise the binding site for the second PCR product and a 3′ SMART primer complementary to the SMART sequence at the 3′ end of the nucleic acid thereby generating the second PCR product, size-selecting a final product comprising the specific gene of interest and determining the genotype of the cell by identifying the UMI and cell BC. Specific sequences can be used to uniquely enable Next Generation Sequencing (NGS) or third-generation sequencing can also be performed by using specific sequences to uniquely enable NGS or third-generation sequencing. Advantageously, the methods allow for determination of expressed DNA sequences, such as mutations, translocations, insertions/deletions (indels), etc.

### Constructing a Library

The methods disclosed herein include a first step of constructing a library, the library includes a plurality of nucleic acids, each nucleic acid including a gene of interest, a unique molecular identifier (UMI) and a cell barcode (cell BC). In a preferred embodiment, each nucleic acid sequence is flanked by switching mechanism at 5′ end of RNA template (SMART) sequences at the 5′ end and 3′ end, that is, in this embodiment, an exemplary nucleic acid in the library would be 5′ SMART-genetic region of interest-UMI-Cell BC-SMART 3′. The libraries can be constructed preferably from any single cell sequencing technique, in some preferred embodiments, an mRNA sequencing protocol, in some embodiments, SMART-Seq. Any single cell sequencing protocol can be used, as described elsewhere herein, to construct the library. In some preferred embodiments, the protocol provides 3′ barcoded nucleic acids that are subjected to further steps in the method embodiments disclosed herein. Additional library construction methods are described elsewhere herein.

### Amplification

Once a library is constructed, an amplifying step is conducted. The amplifying of each nucleic acid in the library can be performed to create first PCR product. In one preferred embodiment, a PCR-amplification based approach is utilized to derive genetic information from single-cell RNA-seq libraries. However, other amplification techniques can be utilized that amplify the library of nucleic acid sequences, with primers designed in accordance with further desired further processing or sequencing techniques, as described herein.

In one particular embodiment, when the libraries are flanked with SMART sequences on both ends, the vast majority of the first PCR product would be amplification of the entire library.

Alternatively, or in addition to and prior to a PCR amplification step, a step of reverse transcription can be performed. In some embodiments, amplifying each nucleic acid in the library to create a whole transcriptome amplified (WTA) RNA by reverse transcription with a primer comprising a sequence adapter. In some embodiments, In certain embodiments, the amplified RNA comprises the orientation: 5′-sequencing adapter-cell barcode-UMI-UUUUUUU-mRNA-3′. In some embodiments, PCR amplification is then conducted of the reverse transcribed products with primers that bind both sequence adapters and adding a library barcode and optionally additional sequence adapters, with subsequent determination of the genotype of the cell by the methods described herein. This particular method can further comprise use of PCR amplification with one or more primers binding both sequence adapters, wherein the one or more primers comprise sequences allowing for circularization of a first PCR product and subsequent circularizing and a second polymerase chain reaction amplification with one or more primers, wherein the one or primers comprise a library barcode and/or additional sequencing adapters.

In some embodiments, any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM).

In specific embodiments, the amplification reaction mixture may further comprise primers, capable of hybridizing to a target nucleic acid strand. The term “hybridization” refers to binding of an oligonucleotide primer to a region of the single-stranded nucleic acid template under the conditions in which primer binds only specifically to its complementary sequence on one of the template strands, not other regions in the template. The specificity of hybridization may be influenced by the length of the oligonucleotide primer, the temperature in which the hybridization reaction is performed, the ionic strength, and the pH. The term “primer” refers to a single stranded nucleic acid capable of binding to a single stranded region on a target nucleic acid to facilitate polymerase dependent replication of the target nucleic acid strand. Nucleic acid(s) that are “complementary” or “complement(s)” are those that are capable of base-pairing according to the standard Watson-Crick, Hoogsteen or reverse Hoogsteen binding complementarity rules.

“PCR” (polymerase chain reaction) refers to a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature greater than 90° C., primers annealed at a temperature in the range 50-75° C., and primers extended at a temperature in the range 72-78° C.

PCR encompasses derivative forms of the reaction, including but not limited to, RT-PCR, real-time PCR, nested PCR, quantitative PCR, multiplexed PCR, and the like. Reaction volumes range from a few hundred nanoliters, e.g., 200 nL, to a few hundred microliters, e.g., 200 microliters. “Reverse transcription PCR,” or “RT-PCR,” means a PCR that is preceded by a reverse transcription reaction that converts a target RNA to a complementary single stranded DNA, which is then amplified, e.g., Tecott et al., U.S. Pat. No. 5,168,038. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“Taqman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture (see, e.g., Bernard et al., Anal. Biochem., 273:221-228, 1999 (two-color real-time PCR)). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Techniques for quantitative PCR are well-known to those of ordinary skill in the art, as exemplified in the following references: Freeman et a1. (Biotechniques, 26:112-126, 1999; Becker-Andre et al. (Nucleic Acids Research, 17:9437-9447, 1989; Zimmerman et al. (Biotechniques, 21:268-279, 1996; Diviacco et al. (Gene, 122:3013-3020, 1992; Becker-Andre et al., (Nucleic Acids Research, 17:9437-9446, 1989); and the like.

### Primers

“Primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of between 3 to 36 nucleotides, from 5 to 24 nucleotides, or from 14 to 36 nucleotides. In certain aspects, primers are universal primers or non-universal primers. Pairs of primers can flank a sequence of interest or a set of sequences of interest. Primers and probes can be degenerate in sequence. In certain aspects, primers bind adjacent to the target sequence, whether it is the sequence to be captured for analysis, or a tag that it to be copied.

In specific embodiments, the amplification reaction mixture may further comprise a first primer and optionally second primer. The first and second primer may comprise a portion that is complementary to a first portion of the target nucleic acid and a second primer comprising a portion that is complementary to a second portion of the target nucleic acid. The first and second primer may be referred to as a primer pair. In some embodiments, the first or second primer may comprise an RNA polymerase promoter.

In specific embodiments, the amplification reaction mixture may further comprise a polymerase. Subsequent to melting and hybridization with a primer, the nucleic acid is subjected to a polymerization step. A DNA polymerase is selected if the nucleic acid to be amplified is DNA. When the initial target is RNA, a reverse transcriptase may first be used to copy the RNA target into a cDNA molecule and the cDNA is then further amplified by a selected DNA polymerase. The DNA polymerase acts on the target nucleic acid to extend the primers hybridized to the nucleic acid templates in the presence of four dNTPs to form primer extension products complementary to the nucleotide sequence on the nucleic acid template.

In some instances, the primer is tagged, in one preferred embodiment, the tagged primer is a 5′ biotinylated primer, typically used with a gene specific sequence in the primer, targeting a gene, mutation, or SNP of interest. In some instances then, a first PCR product is generated by amplifying sequences with a biotinylated 5′ primer comprising a binding site for a second PCR product and a sequence complementary to a specific gene of interest and a 3′ SMART primer complementary to the SMART sequence at the 3′ end of the nucleic acid to generate a first PCR product. The binding site for the second PCR product may be a partial Illumina sequencing primer binding site or an oligomer for sequencing kit, such as a NEBNext® oligos for Illumina® sequencing (see, e.g., neb.com For library preparation for next generation sequencing, Illumina library preparation). However, oligomers for other sequencing kits can be used in the methods described herein, allowing for versatile end use products. Advantageously, nanopore sequencing can also be performed with the methods disclosed herein, with binding sites tailored for such end uses.

The 5′ primer comprising the binding site for the second PCR product to amplify the first PCR product may further comprise a sequence to bind a flow cell, a sequence allowing multiple sequencing libraries to be sequenced simultaneously and/or a sequence providing an additional primer binding site. The sequence to bind a flow cell may be a P7 sequence and the flow cell may be an Illumina® flowcell. In some embodiments where a reverse transcription and subsequent circularization is performed, P5 and P7 are used in primers of a second PCR amplication and size selection. One of skill in the art can adjust the primers based on desired end material when more is needed for example for nanopore sequencing, and for end use, when next generation sequencing is or is not used.

In another embodiment, the SMART primer complementary to the SMART sequence at the 3′ end of the nucleic acid to amplify the first PCR product may further comprise a sequence to allow fragments to bind a flowcell. The sequence to allow fragments to bind a flowcell may be a P5 sequence.

Regardless of the library construction method, submitted libraries may consist of a sequence of interest flanked on either side by adapter constructs. On each end, these adapter constructs may have flow cell binding sites, P5 and P7, which allow the library fragment to attach to the flow cell surface. The P5 and P7 regions of single-stranded library fragments anneal to their complementary oligos on the flowcell surface. The flow cell oligos act as primers and a strand complementary to the library fragment is synthesized. The original strand is washed away, leaving behind fragment copies that are covalently bonded to the flowcell surface in a mixture of orientations. 1,000 copies of each fragment are generated by bridge amplification, creating clusters. Bridge amplification can be performed by methods known in the art, for example, as described in U.S. Pat. No. 7,972,820 and U.S. application Ser. No. 15/316,470. For simplification, the figures diagramming the methods show only one copy (out of 1,000) in each cluster, and only two clusters (out of 30-50 million). The P5 region is cleaved, resulting in clusters containing only fragments which are attached by the P7 region. This ensures that all copies are sequenced in the same direction. The sequencing primer anneals to the P5 end of the fragment, and begins the sequencing by synthesis process. Index reads are only performed when a sample is barcoded. When Read 1 is finished, everything from Read 1 is removed and an index primer is added, which anneals at the P7 end of the fragment and sequences the barcode. Everything is stripped from the template, which forms clusters by bridge amplification as in Read 1. This leaves behind fragment copies that are covalently bonded to the flowcell surface in a mixture of orientations. This time, P7 is cut instead of P5, resulting in clusters containing only fragments which are attached by the P5 region. This ensures that all copies are sequences in the same direction (opposite Read 1). The sequencing primer anneals to the P7 region and sequences the other end of the template.

In another embodiment, the sequence allowing multiple sequencing libraries to be sequenced simultaneously may be an INDEX sequence. The INDEX allows multiple sequencing libraries to be sequenced simultaneously (and demultiplexed using Illumina's bcl2fastq command). See, e.g., https://support.illumina.com for exemplary INDEX sequences.

In another embodiment, the 5′ primer comprising the binding site for the second PCR product to amplify the first PCR product may further comprise a NEXTERA sequence. See, support.illumina.com and U.S. Pat. Nos. 5,965,443, and 6,437,109 and European Patent No. 0927258, for exemplary NEXTERA sequences.

In another embodiment, the sequence providing an additional primer binding site may be a custom readl primer binding site (CR1P) for sequencing. CR1P is a Custom Readl Primer binding site that is used for Drop-Seq and Seq-Well library sequencing. CRIP may comprise the sequence: GCCTGTCCGCGGAAGCAGTGGTATCAACGCAGAGTAC (SEQ ID NO: 1) (see e.g., Gierahn et al., Nature Methods 14, 395-398 (2017).

Biotin-NEXT-GENE-for: Biotinylation enables purification of the desired product following the first PCR reaction. NEXT creates a binding site for the second PCR product as well as a partial primer binding site for standard Illumina sequencing kits. NEXT may be any sequence that allows targeted enrichment and then select addition of sequencing handles. GENE is a sequence complementary to the WTA, designed to amplify a specific region of interest (in some embodiments, an exon).

SMART-rev: The SMART sequence is used in Drop-seq and Seq-Well to generate WTA libraries. Because the polyT-unique molecular identifier-unique cellular barcode (polyT-UMI-CB) sequence is followed by the SMART sequence, and the template switching oligo (TSO) also contains the SMART sequence, WTA libraries have the SMART sequence as a PCR binding site on both the 5′ and the 3′ end.

P7-INDEX-NEXTERA: The P7 sequence allows fragments to bind the Illumina flowcell. The INDEX allows multiple sequencing libraries to be sequenced simultaneously (and demultiplexed using Illumina's bcl2fastq command). The NEXTERA sequence provides a primer binding site for Illumina's standard Read2 sequencing primer mix.

SMART-CR1P-P5: The SMART sequence is the same as in SMART-rev. CRIP is a Custom Read1 Primer binding site that is used for Drop-Seq and Seq-Well library sequencing. The P5 sequence allows fragments to bind the Illumina flowcell. Note that the primer design can be easily modified for compatibility with additional single-cell RNA-seq technologies (SMART) or sequencing technologies (NEXTERA, CRIP).

Gene specific primers may be mixed for simultaneous detection of multiple mutations. Libraries may also be mixed for simultaneous detection of mutations in multiple samples. Mixed primers sometimes may not always detect multiple mutations in the same gene as only the shortest fragment in some instances will be detected. The 5′ primer comprising the binding site for the second PCR product to amplify the first PCR product further comprises a sequence allowing multiple sequencing libraries to be sequenced simultaneously.

### Enrichment

Nucleic acid enrichment reduces the complexity of a large nucleic acid sample, such as a genomic DNA sample, cDNA library or mRNA library, to facilitate further processing and genetic analysis. In certain example embodiments, the enrichment step is optional.

The method also provides for biotin enrichment of the first PCR product. Biotinylation of the primer to amplify the gene, region or mutation of interest from the library allows for the purification of the PCR product of interest. Because the libraries are flanked with SMART sequences on both ends, the vast majority of the first PCR product would be amplification of the entire library. In some embodiments, without the biotinylated primer, enrichment of the gene, region or mutation of interest would be insufficient to efficiently and confidently call genetic mutations. Biotin enrichment may be accomplished by streptavidin binding of the biotinylated first PCR product. The streptavidin bead kilobaseBINDER kit (Thermo Fisher Cat #60101) allows for isolation of large biotinylated DNA fragments. However, as described herein, other embodiments of the methods disclosed herein do not require an enrichment step and may advantageously be used without biotinylated primers.

### Second Amplification

A second step of amplifying may be performed, in a preferred embodiment, a second PCR step is performed. However, in some embodiments, other methods of amplification can be utilized, as discussed herein.

In one embodiment, amplifying the tag-enriched first PCR product with a 5′ primer comprising the binding site for the second PCR product and a 3′ SMART primer complementary to the SMART sequence at the 3′ end of the nucleic acid thereby generating the second PCR product, the SMART primer complementary to the SMART sequence at the 3′ end of the nucleic acid to amplify the first PCR product further comprises a sequence to allow fragments to bind a flowcell. In an embodiment, one of the PCR primers for the second PCR amplification comprises a sequence to allow fragments to bind a flowcell is a P5 sequence, with the second primer comprising a barcoded oiligos that can be used for library indexing. In some instances, the primers comprise a deoxyuracil residue that can be incorporated in the first PCR product such that the first PCR product can be treated with a uracil-specific excision reagent.

In some embodiment, as discussed herein, comprises treating the first PCR product with a uracil-specific excision reagent (“USER®”) enzyme, circularizing the first PCR product by sticky end ligation, and amplifying the tag-enriched circularized PCR product with a 5′ primer complementary to gene of interest and having a sequence adapter and a 3′ primer having a polyA tail and another sequence adapter thereby generating the second PCR product.

Optionally, additional amplification steps can be performed, including a thrif or fourth amplification. In some embodiments, amplification is performed by PCR, and can be utilized when additional material is needed for further manipulation of the libraries, including, for example third generation sequencing. Other amplification methods as described elsewhere herein, can be used with appropriate primers selected according to the amplification methods used, and the final library content desired.

### Determining Genotype

Determining the genotype of the cell may be accomplished by identifying the UMI and cell BC, thereby distinguishing the cells by genotype, or expressed DNA sequences, such as mutations, translocations, insertions/deletions (indels), etc. In one embodiment, the nucleic acids comprise a tag that is a molecule that can be affinity selected such as, but not limited to, a small protein, peptide, nucleic acid. Advantageously, the tag is a biotin tag. The enriched libraries provided by the methods may be further distinguished or manipulated, including by subjecting to sequencing.

In addition to next-generation sequencing, long read/third-generation sequencing is also contemplated for use in the presently disclosed subject matter. Third-generation sequencing reads nucleotide sequences at the single molecule level. In some embodiments, third-generation sequencing is used when long reads are desired, and can be used, in some instances, instead of next-generation sequencing technologies in desired applications. In particular embodiments, nanopore sequencing or single molecule real time sequencing (SMRT) is used for third-generation sequencing. Nanopore technology libraries are generated by end-repair and sequencing adapter ligation, and, as such, allows for versatility in the sequencing adapters utilized in the PCR reaction. Accordingly, in some instances, when nanopore sequencing is utilized, the ‘sequencing adapters’ in the first PCR reaction is any adapter that allows for a second PCR with common primers. Exemplary nanopore technology that can be used for long reads can be found, for example, using Oxford Nanopore technology, available at nanoporetech.com. Long-read sequencing can also utilize SMRT sequencing which enables single-molecule resolution through the use of nucleotides uniquely labeled with a fluorophore, and observing a single DNA polymerase molecule while synthesizing a complementary DNA in a replication reaction to allow for single molecule resolution. tallows production of a natural DNA strand using the labeled nucleotides. In some instances, when third-generation sequencing will be used, additional amplification can be performed to generate sufficient material.

### Distinguishing Cells by Genotype

A method of distinguishing cells by genotype may, in some embodiments comprise constructing a library as discussed herein that comprises a plurality of nucleic acids wherein each nucleic acid comprises a gene, a unique molecular identifier (UMI) and a cell barcode (cell BC) flanked by sequencing adapters at the 5′ and 3′ end. In particular embodiments, each nucleic acid comprises the orientation: 5′-sequencing adapter-cell barcode-UMI-UUUUUUU-mRNA-3′. Amplifying each nucleic acid in the library to create a whole transcriptome amplified (WTA) RNA by reverse transcription can be performed with a primer comprising a sequence adapter to provide a reverse transcribed product. The steps provide amplifying the reverse transcribed product by PCR amplification with primers that bind both sequence adapters and adding a library barcode and optionally additional sequence adapters to generate a first PCR product. The genotype of the cell can be performed as discussed elsewhere, including identifying the UMI and library barcode, thereby distinguishing the cells by genotype.

### Reverse Transcribing

In specific embodiments, the amplification reaction mixture may further comprise a polymerase. Subsequent to melting and hybridization with a primer, the nucleic acid is subjected to a polymerization step. A DNA polymerase is selected if the nucleic acid to be amplified is DNA. When the initial target is RNA, a reverse transcriptase may first be used to copy the RNA target into a cDNA molecule and the cDNA is then further amplified by a selected DNA polymerase. The DNA polymerase acts on the target nucleic acid to extend the primers hybridized to the nucleic acid templates in the presence of four dNTPs to form primer extension products complementary to the nucleotide sequence on the nucleic acid template.

Optionally Treating with USER Enzyme and Amplifying

In some embodiments, the primers for amplifying in in a first PCR amplification comprise USER sequences, and further comprising treating the first PCR product with USER enzyme, thereby generating a circularized product.

The steps include cleaving the dU residue by addition of a uracil-specific excision reagent (“USER®”) enzyme/T4 ligase to generate long complementary sticky ends to mediate efficient circularization and ligation, which now places the barcode and the 5′ edge of the transcript sequence set in the primer extension in close proximity, thereby bringing the cell barcode within 100 bases of any desired sequence in the transcript.

Following treating with USER enzyme, the step of amplifying the circularized product in a second polymerase chain reaction with one or more primers, wherein the one or primers comprise a library barcode and/or additional sequencing adapters can be conducted.

In some embodiments, the method can then include more than one PCR steps with transcript specific primers, that can include adaptor sequences, and preferably uses nested PCR reactions where the final PCR reaction sets the 3′ edge of the transcript sequence of the final sequencing construct. The final sequencing library can be utilized in several ways, including sequencing of the transcript sequence, or at some desired location in the transcript sequence.

Circularization without Enrichment

In one embodiment, the methods disclosed herein provide a protocol that eliminates need for enrichment in a scalable process. An exemplary embodiment can provide for amplification of all variable regions of a T-cell receptor. The methods described herein can be advantageously be used for the amplification of regions not well characterized in RNA seq libraries. The steps include providing an RNAseq library, in some preferred embodiments, a SeqWell library. The starting library comprises a plurality of nucleic acids with each nucleic acid comprising a gene, a unique molecular identifier (UMI) and a cell barcode (cell BC) flanked by universal sequences.

In an embodiment, the method comprises conducting primer extension on a nucleic acid in the library with one or more 5′ primers with each primer comprising a sequence complementary to a desired transcript and the universal sequence of the nucleic acid, thereby replicating one or more desired transcripts and setting a 5′ edge of one or more desired transcript sequences in one or more final sequencing constructs; amplifying the replicated one or more desired transcript sequences with universal primers having complementary sequences on 5′ ends of the universal primers followed by a deoxy-uracil residue to form an amplicon; and ligating the amplicons by reacting the amplicons with a uracil-specific excision reagent enzyme, thereby cleaving the amplicon at the deoxy-uracil residues resulting in sticky ends that mediate circularization.

Additional steps of amplifying by PCR may be performed. In these instances, primers complementary to a transcript of interest. In some preferred embodiments, at least two PCR steps are performed in a nested PCR using two sets of transcript specific primers complementary to a transcript of interest. As described previously, the primers may comprise adaptor sequences. In one embodiment, at least one set of the two sets of transcript specific primers comprise adaptor sequences, thereby yielding a final sequencing library of final sequencing constructs. In an embodiment, the last PCR step sets a 3′ edge of the transcript sequence of the final construct. In some embodiments, the sequencing step utilizes primers complementary to the 3′ set and 5′ set edges of the final sequencing construct. The sequencing step can utilize a primer binding to a desired location in the final sequencing construct to drive a sequencing read at the desired location in the final sequencing construct, as described elsewhere herein.

The embodiments disclosed herein method works particularly well for libraries where a subset of the transcripts of interest are more than 1 kb away from the cell barcode. Particularly, variable regions of T-cell receptors can be used in the current methods. Accordingly, the transcript of interest can be in a T cell or a B cell, in some embodiments, in a T cell receptor, a B cell receptor or a CAR-T cell. Advantageously, the embodiment can comprise use of a pool of primers that, in an embodiment targeting variable regions, may target all variable regions. The sequencing method may also determine SNPs in the single cell.

### RNA-Seq/Single Cell Sequencing

As described above, in some embodiments, gene expression can be determined using an RNA-seq-based method. In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi: 10. 1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aabl601. Epub 2015 May 7; US20160208323A1; US20160060691A1; and WO2017156336A1).

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, N.Y. (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS). sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (1¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Methods of Modulating and Engineering Bone Marrow Stromal Cells

Described herein are methods of modulating stromal from one cell state and/or type to another. In some embodiments, the method can include modulating a cell or population thereof that is in a disease-associated cell state to a homeostatic or normal cell state. The methods of modulating stromal cells described herein can be used, for example, to engineer stromal cells having a particular cell state and corresponding characteristics and attributes, to screen and identify agents capable of inducing a particular cell state, and/or for the treatment of disease among others. These and other applications, features, and advantages for/of the methods of modulating stromal cells are described in greater detail elsewhere herein.

### Methods of Modulating Bone Marrow Stromal Cell State

Described elsewhere herein are bone marrow stromal and/or immune cells that can be modified or engineered to express a particular gene, signature (e.g. a gene signature). Such modification and/or engineering can occur ex vivo and/or in vivo. Not being bound by a theory, modifying immune and/or other cells (e.g. other stromal cells) in vivo, such that dysfunctional cells are decreased can provide a therapeutic effect, including but not limited to enhancing an immune response and/or remodeling the bone marrow stromal cell landscape, and/or remodeling the bone marrow microenvironment in a subject. A gene, gene signature, bone marrow stromal cell, or immune cell may be modified by any suitable modulating agent. Methods of modulating cells, screening and identifying suitable modulating agents, and suitable modulating agents are described in greater detail elsewhere herein.

The invention further relates to agents capable of inducing or suppressing particular stromal cell (sub)populations based on the gene signatures, protein signature, and/or other genetic or epigenetic signature as defined herein, as well as their use for modulating, such as inducing or repressing, a particular gene signature, protein signature, and/or other genetic or epigenetic signature. In one embodiment, genes in one population of cells may be activated or suppressed in order to affect the cells of another population. In related aspects, modulating, such as inducing or repressing, a particular a particular gene signature, protein signature, and/or other genetic or epigenetic signature may modify overall stromal cell composition, such as stromal cell composition (such as in an adoptive cell therapy), such as stromal cell subpopulation composition or distribution, or functionality.

The terms, “cell landscape”, “cellular landscape”, are used interchangeably herein to refer to the possible and/or actual profile of cell states and/or cell types present within a defined cell population, such as a tissue, sample, organ, system, and the like. For example, in some embodiments the stromal cell landscape can include cells in various states, such as cell states defined by signatures of Clusters 1-17. Remodeling of the cellular landscape can occur by various methods, such that the relative number of each cell state and/or cell type within the defined cell population is changed. This can occur, for example, by adding and/or removing cells of a specific cell state and/or type from the defined cell population and/or modulating the signatures of one or more cells such that they shift cell state and thus alter the relative number of each cell in the defined population. In some aspects, diseases can result in remodeling a cell landscape such that the cell landscape is pathogenic or supportive of a disease state and/or disease development. In some aspects, a diseased cell landscape can be remodeled such that it is no longer diseased but is like or more like a homeostatic and/or beneficial cell landscape.

In some embodiments the method of modifying cells states in stromal cells can include administering a modulating agent to a subject or cell population that induces a shift in stromal cells from a disease cell state to a homeostatic or a normal cell state. In some aspects, the stromal cell-state and/or type is characterized by expression of the genes any one of Tables 1-8 or a combination thereof described or as otherwise identified in Clusters 1-17 or a subtype thereof as demonstrated in the Working Examples below or an expression signature derived therefrom. In some aspects, the shift in cell state comprises reducing the distance in gene expression space between the disease-associated cell state and the homeostatic stromal cell state. In some aspects, identifying differences in cell state between the dysfunctional and the homeostatic cell states comprises comparing a gene expression distribution of dysfunctional stromal cells with a gene expression distribution of homeostatic and/or activated as determined by single cell RNA sequencing (scRNA-seq). In some aspects, wherein the gene expression space comprises 10 or more genes, 20 or more genes, 30 or more genes, 40 or more genes, 50 or more genes, 100 or more genes, 500 or more genes, or 1000 or more genes. In some aspects, wherein modulation comprises increasing or decreasing expression of one or more genes, gene expression cassettes, or gene expression signatures.

In aspects, the cell population can be composed of comprises a single cell type and/or subtype, a combination of cell types and/or subtypes, a cell-based therapeutic, an explant, or an organoid. In some aspects, the cell population comprises bone marrow stromal cells.

In some aspects, a method of screening for one or more agents capable of modulating stromal cell states, can include: contacting a cell population comprising stromal cells having an initial cell state with a test modulating agent or library of modulating agents; determining a fraction of stromal cell states including a fraction of homeostatic and dysfunctional stromal cells and selecting modulating agents that shift the initial stromal cell state to a desired stromal cell state where the desired stromal fraction in the cell population is above a set cutoff limit.

In some aspects, the initial cell state is a stromal cell state and the desired cell state is a homeostatic cell state. In some aspects, wherein the cell population is obtained from a subject to be treated.

Embodiments disclosed herein provide for isolated ex vivo systems that can include one or more cells of a particular cell identity, type, and/or state and formulations thereof. Also provided herein are methods of generating and using the cells, cell-based systems, populations, and formulations thereof. In aspects, the cells and/or ex vivo cell-based systems can recapitulate an in vivo phenotype, which can include a particular cell identity, type, and/or state. As used herein, to “recapitulate an in vivo phenotype” may include increasing the biological fidelity of a cell or population thereof and/or an ex vivo cell-based system to more closely mimic the cell identity, cell type, cell state, physiology and/or structure of a in vivo target or reference cell or system. Mimicking the physiology and/or structure of in vivo target or reference cell or system can include mimicking expression signatures or modules found in vivo target or reference cell or system, mimicking a cell state or states found in the in vivo target or reference cell or system, mimicking the composition of cell types or sub-types found in the in vivo target or reference cell or system, and/or mimicking the a cell identity or identities found in the in vivo target or reference cell or system. In some aspects, the in vivo target or reference cell or system (e.g. stromal cell or system thereof) can have a homeostatic cell state or an activated cell state. Described elsewhere herein, are methods of identifying stromal cells and populations thereof having a specific cell state (e.g. any one of clusters 1-17 or subtypes within a cluster as described elsewhere herein), which can be used to identify the state of the stromal cell. An “ex vivo cell-based system” may be composed of single cells of a particular type, sub-type or state, or a combination of cells of the same or differing type, sub-type, or state. The ex vivo cell-based system may be a model for screening perturbations to better understand the underlying biology or to identify putative targets for treating a disease, or for screening putative therapeutics, and also include models derived ex vivo but further implanted into a living organism, such as a mouse or pig, prior to perturbation of the model. An ex vivo cell-based system may also be a cell-based therapeutic for delivery to an organism to treat disease, or an implant meant to restore or regenerate damaged tissue. Ex vivo cell-based systems can include isolated and/or engineered cells, such as isolated and/or engineered cell-based systems. An “in vivo system” may likewise comprise a single cell or a combination of cells of the same or differing type, sub-type, or state. As used herein ex vivo may include, but not be limited to, in vitro systems, unless otherwise specifically indicated. The “in vivo system” may comprise healthy tissue or cells, or tissues or cells in a homeostatic state, or diseased tissue or cells, or diseased tissue or cells in a non-homeostatic state, or tissues or cells within a viable organism, or diseased tissue or cells within a viable organism. A homeostatic state may include cells or tissues demonstrating a physiology and/or structure typically observed in a healthy living organism. In other embodiments, a homeostatic state may be considered the state that a cell or tissue naturally adopts under a given set of growth conditions and absent further defined genetic, chemical, or environmental perturbations.

Current in vitro models used to look at biology are not well characterized with reference to in vivo models. The embodiments disclosed herein provide a means for identifying differences in expression at a single cell level and use this information to prioritize how to improve the ex vivo system to more faithfully recapitulate the biological characteristics of the target in vivo system. Particular advantageous uses for ex vivo cell-based systems that faithfully recapitulate an in vivo phenotype of interest include methods for identifying agents capable of inducing or suppressing certain gene signatures or gene expression modules and/or inducing or suppressing certain cell states in the ex vivo cell-based systems. In the context of cell-based therapeutics, the methods disclosed herein may also be used to design ex vivo cell-based systems that based on their programmed gene expression profile or configured cell state can either induce or suppress particular in vivo cell (sub)populations at the site of delivery. In another aspect, the methods disclosed herein provide a method for preparing cell-based therapeutics.

In certain example embodiments, a method for generating an ex vivo cell-based system that faithfully recapitulates an in vivo phenotype or target system of interest comprises first determining, using single cell RNA sequencing (scRNA-seq) one or more cell (sub)types or one or more cell states in an initial or starting ex vivo cell-based system. It should be noted that the methods disclosed herein may be used to develop an ex vivo cell-based system de novo from a source starting material, or to improve an existing ex vivo cell-based system. Source starting materials may include cultured cell lines or cells or tissues isolated directly from an in vivo source, including explants and biopsies. The source materials may be pluripotent cells including stem cells. Next, differences are identified in the cell (sub)type(s) and/or cell state(s) between the ex vivo cell-based systems a target in vivo system. The cell (sub)type(s) and cell state(s) of the in vivo system may likewise be determined using scRNA-seq or other suitable technique. The scRNA-seq analysis (or other appropriate analysis) may be obtained at the time of running the methods described herein are based on previously archived scRNA-seq analysis. Based on the identified differences, steps to modulate the source material to induce a shift in cell (sub)type(s) and/or cell state(s) that may more closely mimics the target in vivo system may then be selected and applied. Various RNA-seq and other suitable techniques and analyses are described in greater detail elsewhere herein.

In certain example embodiments, assessing the cell (sub)types and states present in the in vivo system may comprise analysis of expression matrices from the scRNA-seq data, performing dimensionality reduction, graph-based clustering and deriving list of cluster-specific genes in order to identify cell types and/or states present in the in vivo system. These marker genes may then be used throughout to relate the ex vivo system cell (sub)types and states to the in vivo system. The same analysis may then be applied to the source material for the ex vivo cell-based system. From both sets of sc-RNAseq analysis an initial distribution of gene expression data is obtained. In certain embodiments, the distribution may be a count-based metric for the number of transcripts of each gene present in a cell. Further the clustering and gene expression matrix analysis allow for the identification of key genes in the initial ex vivo system and the target in vivo system, such as differences in the expression of key transcription factors. In certain example embodiments, this may be done conducting differential expression analysis.

For example, in the Working Examples below, differential gene expression analysis identified that stromal cells from bone marrow can be distinguished into 17 types with more subtypes within at least some of the types. Further, some cell states are associated with a diseased state and/or a remodeled bone marrow microenvironment, which can support or facilitate disease development. Thus, the methods disclosed herein can both identify key markers of diseased or dysfunctional stromal cells, as well as different normal or healthy cells sates and types, which can be potential targets for modulation to shift the expression distribution of the ex vivo system towards that of the target in vivo or non-diseased system.

Other methods for assessing differences in the ex vivo and in vivo systems may be employed. In certain example embodiments, an assessment of differences in the in vivo and ex vivo proteome may be used to further identify key differences in cell type and sub-types or cells. states. For example, isobaric mass tag labeling and liquid chromatography mass spectroscopy may be used to determine relative protein abundances in the ex vivo and in vivo systems. The working examples below provide further disclosure on leveraging proteome analysis within the context of the methods disclosed herein. In certain example embodiments, a statistically significant shift in the initial ex vivo gene expression distribution toward the gene expression distribution of the in vivo systems is sought post-modulation. This is described in greater detail herein with respect to “engineered stromal cells” or “modified stromal cells”.

In certain embodiments, the method may further comprise modulating the initial cell-based system to induce a gain of function in addition to the in vivo phenotype of interest comprising modulating expression of one or more genes, gene expression cassettes, or gene expression signatures associated with the gain of function. In certain embodiments, the method may further comprise modulating the initial cell-based system to induce a loss of function in addition to the in vivo phenotype of interest comprising modulating expression of one or more genes, gene expression cassettes, or gene expression signatures associated with the loss of function.

In certain embodiments, modulating comprises increasing or decreasing expression of one or more genes, gene expression cassettes, or gene expression signatures. In certain embodiments, modulating includes activating or inhibiting one or more genes, gene expression cassettes, or gene expression signatures (e.g., with an agonist or antagonist). In certain embodiments, modulating the initial cell-based system comprises delivering one or more modulating agents that modify expression of one or more cell types or states in the initial cell-based system, delivering an additional cell type or sub-type to the initial cell-based system, or depleting an existing cell type or sub-type from the initial cell-based system. The one or more modulating agents may comprise one or more cytokines, growth factors, hormones, transcription factors, metabolites or small molecules. The one or more modulating agents may be a genetic modifying agent or an epigenetic modifying agent. The genetic modifying agent may comprise a CRISPR system, a zinc finger nuclease system, a TALEN, or a meganuclease. The epigenetic modifying agent may comprise a DNA methylation inhibitor, HDAC inhibitor, histone acetylation inhibitor, histone methylation inhibitor or histone demethylase inhibitor.

In certain embodiments, the one or more modulating agents modulate one or more cell-signaling pathways. The one or more pathways may comprise Notch signaling. The one or pathways may comprise Wnt signaling.

In certain embodiments, the initial cell-based system comprises a single cell type or sub-type, a combination of cell types and/or subtypes, cell-based therapeutic, an explant, or an organoid.

In certain embodiments, the single cell type or subtype or combination of cell types and/or subtypes comprises an immune cell, intestinal cell, liver cell, kidney cell, lung cell, brain cell, epithelial cell, endoderm cell, neuron, ectoderm cell, islet cell, acinar cell, oocyte, sperm, blood cell, hematopoietic cell, hepatocyte, skin/keratinocyte, melanocyte, bone/osteocyte, hair/dermal papilla cell, cartilage/chondrocyte, fat cell/adipocyte, skeletal muscular cell, endothelium cell, cardiac muscle/cardiomyocyte, neuronal cells, non-neuronal cells, trophoblast, tumor cell, or tumor microenvironment (TME) cell.

In certain embodiments, the initial cell-based system is derived from a subject with a disease (e.g., to study the disease ex vivo). The disease can be a hematological disease. Such diseases are described in greater detail herein.

In some embodiments, a method of generating an engineered stromal cell can include first determining, using single cell RNA sequencing (scRNA-seq) one or more cell (sub)types or one or more cell states in an initial or starting ex vivo cell-based system. It should be noted that the methods disclosed herein may be used to develop an ex vivo cell-based system de novo from a source starting material, or to improve an existing ex vivo cell-based system. Source starting materials may include cultured cell lines or cells or tissues isolated directly from an in vivo source, including explants and biopsies. The source materials may be pluripotent cells including stem cells. Next, differences are identified in the cell (sub)type(s) and/or cell state(s) between the ex vivo cell-based systems a target in vivo system. The cell (sub)type(s) and cell state(s) of the in vivo system may likewise be determined using scRNA-seq. The scRNA-seq analysis may be obtained at the time of running the methods described herein are based on previously archived scRNA-seq analysis. Based on the identified differences, steps to modulate the source material to induce a shift in cell (sub)type(s) and/or cell state(s) that may more closely mimics the target in vivo system may then selected and applied.

In certain embodiments, different methods of single sequencing are better suited for sequencing certain samples (e.g., neurons, rare samples may be more optimally sequenced with a plate-based method or single nuclei sequencing). In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO 2014210353 A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain example embodiments, assessing the cell (sub)types and states present in the in vivo system may comprise analysis of expression matrices from the scRNA-seq data, performing dimensionality reduction, graph-based clustering and deriving list of cluster-specific genes in order to identify cell types and/or states present in the in vivo system. These marker genes may then be used throughout to relate the ex vivo system cell (sub)types and states to the in vivo system. The same analysis may then be applied to the source material for the ex vivo cell-based system. From both sets of sc-RNAseq analysis an initial distribution of gene expression data is obtained. In certain embodiments, the distribution may be a count-based metric for the number of transcripts of each gene present in a cell. Further the clustering and gene expression matrix analysis allow for the identification of key genes in the initial ex vivo system and the target in vivo system, such as differences in the expression of key transcription factors. In certain example embodiments, this may be done conducting differential expression analysis.

Other methods for assessing differences in the ex vivo and in vivo systems may be employed. In certain example embodiments, an assessment of differences in the in vivo and ex vivo proteome may be used to further identify key differences in cell type and sub-types or cells. states. For example, isobaric mass tag labeling and liquid chromatography mass spectroscopy may be used to determine relative protein abundances in the ex vivo and in vivo systems. The working examples below provide further disclosure on leveraging proteome analysis within the context of the methods disclosed herein.

In certain example embodiments, a statistically significant shift in the initial ex vivo gene expression distribution toward the gene expression distribution of the in vivo systems is sought post-modulation. A statistically significant shift in gene expression distribution can be at least 10%, at least 11%, at least 12%, at least 13%, at least 14%, at least 15%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In certain example embodiments, statistical shifts may be determined by defining an in vivo score. For example, a gene list of key genes enriched in the in vivo model may be defined. To determine the fractional contribution to a cell's transcriptome to that gene list, the total log (scaled UMI+1) expression values for gene with the list of interest are summed and then divided by the total amount of scaled UMI detected in that cell giving a proportion of a cell's transcriptome dedicated to producing those genes. Thus, statistically significant shifts may be shifts in an initial score for the ex vivo system after modulation towards the in vivo score or after modulation with an aim of moving in a statistically significant fashion towards the in vivo score.

Modulation may be monitored in a number of ways. For example, expression of one or more key marker genes identified as described above may be measured at regular levels to assess increases in expression levels. Shifting of the ex vivo system to that of the in vivo system may also be measured phenotypically. For example, imaging an immunocytochemistry for key in vivo markers may be assessed at regular intervals to detect increased expression of the key in vivo markers. Likewise, flow cytometry may be used in a similar manner. In addition, to detecting key in vivo markers, imaging modalities such as those described above may be used to further detect changes in cell morphology of the ex vivo system to more closely resemble the target in vivo system.

In certain example embodiments, the ex vivo system may be further modulated to not only more faithfully recapitulate a target in vivo system, but the ex vivo system may be further modulated to induce a gain of function. For example, one or more genes, gene expression cassettes (modules), or gene expression signature associated with the gain of function may be induced. Example gain of functions include, but are not limited to, increased anti-apoptotic activity or improved anti-microbial secretion.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

In further aspects, the invention relates to gene signatures, protein signature, and/or other genetic or epigenetic signature of particular bone marrow stromal cell subpopulations, as defined herein elsewhere. The invention hereto also further relates to particular bone marrow stromal cell subpopulations, which may be identified based on the methods according to the invention as discussed herein; as well as methods to obtain such cell (sub)populations and screening methods to identify agents capable of inducing or suppressing particular tumor cell (sub)populations.

In some exemplary embodiments, described herein are methods of remodeling a stromal cell landscape comprising administering a modulating agent to a subject or a cell population that induces a shift in the stromal cell landscape from a disease-associated stromal cell landscape to a homeostatic stromal cell landscape.

In some exemplary embodiments, the shift in stromal cells from a disease-associated stromal cell landscape to a homeostatic stromal cell landscape comprises a change in the proportion of preosteoblasts. In some exemplary embodiments, the change in the proportion of preosteoblasts comprises a change in the relative proportion of OLC-1 cells to OLC-2 cells. In some exemplary embodiments, the change in the relative proportion of OLC-1 cells to OLC-2 cells comprises a decrease in OLC-1 cells and an increase in OLC-2 cells.

In some exemplary embodiments, the shift in stromal cells from a disease-associated stromal cell landscape to a homeostatic stromal cell landscape comprises a change in the relative proportion of bone marrow derived endothelial cell subtypes. In some exemplary embodiments, the change in the relative proportion of bone marrow derived endothelial cell subtypes comprises an increase in sinusoidal bone marrow derived endothelial cells and a decrease in arterial bone marrow derived endothelial cells.

In some exemplary embodiments, the shift in stromal cells from a disease-associated stromal cell landscape to a homeostatic stromal cell landscape comprises a change in the relative proportion of chondrocyte subtypes. In some exemplary embodiments, the change in the relative proportion of chondrocyte subtypes comprises a decrease in chondrocyte hypertrophic cell subtype and an increase in chondrocyte progenitor cell subtype.

In some exemplary embodiments, the shift in stromal cells from a disease-associated stromal cell landscape to a homeostatic stromal cell landscape comprises a change in the relative proportion of fibroblast subtypes. In some exemplary embodiments, the change in the relative proportion of fibroblast subtypes comprises an increase in fibroblast subtype-3 and a decrease in fibroblast subtype-4.

In some exemplary embodiments, the shift in stromal cells from a disease-associated stromal cell landscape to a homeostatic stromal cell landscape comprises a change in the relative proportion in mesenchymal stem/stromal cell (MSC) subtypes. In some exemplary embodiments, the change in the relative proportion in mesenchymal stem/stromal cell (MSC) sub-types comprises a decrease in MSC-2 subtype and an increase in MSC-3 and MSC-4 subtypes.

In some exemplary embodiments, the shift in the stromal cell landscape comprises a change in the distance in gene expression space between OLC-1, OLC-2, bone marrow derived endothelial cell subtypes, chondrocyte subtypes, fibroblast subtypes, mesenchymal stem/stromal cell (MSC) subtypes, or a combination thereof. In some exemplary embodiments, the distance is measured by a Euclidean distance, Pearson coefficient, Spearman coefficient, or a combination thereof. In some exemplary embodiments, the gene expression space comprises 10 or more genes, 20 or more genes, 30 or more genes, 40 or more genes, 50 or more genes, 100 or more genes, 500 or more genes, or 1000 or more genes. In some exemplary embodiments, remodeling the stromal cell landscape comprises increasing or decreasing the expression of one or more genes, gene programs, gene expression cassettes, gene expression signatures, or a combination thereof. In some exemplary embodiments, the change in the gene expression space is characterized by a change in the expression of one or more genes as in any of Tables 1-8 or an expression signature derived therefrom. In some exemplary embodiments, identifying differences in stromal cell states in the shift in the stromal cell landscape comprises comparing a gene expression distribution of a stromal cell type or subtype in the diseased stromal cell landscape with a gene expression distribution of the stromal cell type or subtype in the homeostatic stromal cell landscape as determined by single cell RNA-sequencing (scRNA-seq).

In some exemplary embodiments, the shift in the stromal cell landscape from a disease-associated stromal cell landscape to a homeostatic stromal cell landscape increases committed MSCs and decreases osteoprogenitor cells.

In some exemplary embodiments, the disease is a hematological disease. In some exemplary embodiments, the hematological disease is a hematopoietic disease. In some exemplary embodiments, the hematological disease is a blood cancer. In some embodiments, the blood cancer is leukemia. In some embodiments, the blood cancer is acute lymphocytic leukemia, acute myeloid leukemia, chronic lymphocytic leukemia, chronic myeloid leukemia, hairy cell leukemia, myelodysplastic syndromes, acute promyelocytic leukemia, or myeloproliferative neoplasm.

### Modulating Agents

Modulating agents are any agents that is capable of directly or indirectly modulate the genome, epigenome, gene expression, signature (e.g. a gene signature), gene module, gene product production, or any other phenotype and/or functionality of a cell, such as a bone marrow stromal cell and/or immune cell described herein. Suitable modulating agents include, but are not limited to, biologic molecules, therapeutic antibodies, antibody fragments, antibody-like protein scaffolds, aptamers, polypeptides, genetic modifying agents, small molecule compounds, small molecule degraders, and combinations thereof. Exemplary biologic molecules that can be suitable modulating agents can include, but are not limited to, cytokines, growth factors, hormones, transcription factors, metabolite, and combinations thereof.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of a cell or cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place. Agents can include any potential class of biologically active conditions, substances or agents, such as for instance antibodies, proteins, peptides, nucleic acids, oligonucleotides, small molecules, or combinations thereof, as described herein.

### Genetic/Epigenetic Modifying Agents

In some embodiments, the modulating agent can be a genetic or epigenetic modifying agent. Suitable genetic modifying agents include, but are not limited to, a CRISPR-Cas system, a zinc finger nuclease system, a TALEN or TALEN system, a meganuclease, an RNAi system, or a combination thereof. Suitable epigenetic modifying agents can include, but are not limited to, a DNA methylation inhibitor, HDAC inhibitor, histone acetylation inhibitor, histone methylation inhibitor or histone demethylase inhibitor.

### CRISPR-Cas Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein His A, CorU.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. a1. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar. oxfordj ournals.org/content/34/7/e53. short and nature.com/mt/j ournal/v 16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Additional effectors for use according to the invention can be identified by their proximity to casl genes, for example, though not limited to, within the region 20 kb from the start of the casl gene and 20 kb from the end of the casl gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

### CRISPR RNA-Targeting Effector Proteins

In some embodiments, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023, which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprises one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/naturel9802 and Abudayyeh et al. “C2c2 is a single-component programmable RNA-guided RNA targeting CRISPR effector” bioRxiv doi: 10.1101/054742.

In certain embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia, wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCas13d) or Ruminococcus sp. N15. MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytindine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g, Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, DA., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi: 10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10. 1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/naturel5521. Epub 2015 Sep. 16.
Cpfl Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et a1, “Engineered Cpfl Enzymes with Altered PAM Specificities,” bioRxiv 091611; doi: http://dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.

each of which is incorporated herein by reference, may be considered in the practice of the instant invention, and discussed briefly below:

Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.

Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.

Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.

Konermann et al. (2013) addressed the need in the art for versatile and robust technologies that enable optical and chemical modulation of DNA-binding domains based CRISPR Cas9 enzyme and also Transcriptional Activator Like Effectors

Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.

Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.

Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.

Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF 1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.

Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.

Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.

Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.

Hsu et al. (2014) is a review article that discusses generally CRISPR-Cas9 history from yogurt to genome editing, including genetic screening of cells.

Wang et al. (2014) relates to a pooled, loss-of-function genetic screening approach suitable for both positive and negative selection that uses a genome-scale lentiviral single guide RNA (sgRNA) library.

Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

Swiech et al. demonstrate that AAV-mediated SpCas9 genome editing can enable reverse genetic studies of gene function in the brain.

Konermann et al. (2015) discusses the ability to attach multiple effector domains, e.g., transcriptional activator, functional and epigenomic regulators at appropriate positions on the guide such as stem or tetraloop with and without linkers.

Zetsche et al. demonstrates that the Cas9 enzyme can be split into two and hence the assembly of Cas9 for activation can be controlled.

Chen et al. relates to multiplex screening by demonstrating that a genome-wide in vivo CRISPR-Cas9 screen in mice reveals genes regulating lung metastasis.

Ran et al. (2015) relates to SaCas9 and its ability to edit genomes and demonstrates that one cannot extrapolate from biochemical assays.

Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.

Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.

Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.

Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.

Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.

Zetsche et al. (2015) reported characterization of Cpfl, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.

Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2cl depends on both crRNA and tracrRNA for DNA cleavage. The third enzyme (C2c2) contains two predicted HEPN RNase domains and is tracrRNA independent.

Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.

Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et a1. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokIl Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 15, PROTECTED GUIDE RNAS (PGRNAS); U.S. application 62/091,455, filed, 12 Dec. 14, PROTECTED GUIDE RNAS (PGRNAS); U.S. application 62/096,708, 24 Dec. 14, PROTECTED GUIDE RNAS (PGRNAS); U.S. applications 62/091,462, 12 Dec. 14, 62/096,324, 23 Dec. 14, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 14 and 62/180,692, 17 Jun. 2015, ESCORTED AND FUNCTIONALIZED GUIDES FOR CRISPR-CAS SYSTEMS; U.S. application 62/091,461, 12-Dec-14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 14, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 14, 62/181,641, 18 Jun. 2015, and 62/181,667, 18 Jun. 2015, RNA-TARGETING SYSTEM; U.S. application 62/096,656, 24 Dec. 14 and 62/181,151, 17 Jun. 2015, CRISPR HAVING OR ASSOCIATED WITH DESTABILIZATION DOMAINS; U.S. application 62/096,697, 24 Dec. 14, CRISPR HAVING OR ASSOCIATED WITH AAV; U.S. application 62/098,158, 30 Dec. 14, ENGINEERED CRISPR COMPLEX INSERTIONAL TARGETING SYSTEMS; U.S. application 62/151,052, 22 Apr. 15, CELLULAR TARGETING FOR EXTRACELLULAR EXOSOMAL REPORTING; U.S. application 62/054,490, 24 Sep. 14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 14, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 14, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 14 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 14, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 14, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 14, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 14 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 14, FUNCTIONAL SCREENING WITH OPTIMIZED FUNCTIONAL CRISPR-CAS SYSTEMS; U.S. application 62/087,546, 4 Dec. 14 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 14, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly these polypeptides can be used for the delivery of CRISPR-effector based RNPs in eukaryotic cells.

### Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoide cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemicially modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemicially modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (melΨ), 5-methoxyuridine(5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU (SEQ ID NOs: 3-6).

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas proten (Chen et al. Cell. (2013); 155(7): 1479-1491). In particular embodiments the stem of the stemloop is extended by at least 1, 2, 3, 4, 5 or more complementary basepairs (i.e. corresponding to the addition of 2,4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the compelementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10. 1038/naturel4592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB 1. Blue light stimulation induces an activating conformational change in cryptochrome-2, resulting in recruitment of its binding partner CIB 1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., www.nature.com/nmeth/j ournal/v2/n6/full/nmeth763.html), 3. GID 1-GAI based system inducible by Gibberellin (GA) (see, e.g., www.nature. com/nchembio/j ournal/v8/n5/full/nchembio. 922.html).

A chemical inducible system can be an estrogen receptor (ER) based system inducible by 4-hydroxytamoxifen (4OHT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogren receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel-based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et a11998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Naturel2466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, TJ., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh 00, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91(2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/naturel5521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et a1, “Engineered Cpf1 Enzymes with Altered PAM Specificities,” bioRxiv 091611; doi: http://dx.doi.org/10.1101/091611 (Dec. 4, 2016).

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2cl depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et a1. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokIl Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 15, PROTECTED GUIDE RNAS (PGRNAS); U.S. application 62/091,455, filed, 12 Dec. 14, PROTECTED GUIDE RNAS (PGRNAS); U.S. application 62/096,708, 24 Dec. 14, PROTECTED GUIDE RNAS (PGRNAS); U.S. applications 62/091,462, 12 Dec. 14, 62/096,324, 23 Dec. 14, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 14 and 62/180,692, 17 Jun. 2015, ESCORTED AND FUNCTIONALIZED GUIDES FOR CRISPR-CAS SYSTEMS; U.S. application 62/091,461, 12 Dec. 14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 14, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 14, 62/181,641, 18 Jun. 2015, and 62/181,667, 18 Jun. 2015, RNA-TARGETING SYSTEM; U.S. application 62/096,656, 24 Dec. 14 and 62/181,151, 17 Jun. 2015, CRISPR HAVING OR ASSOCIATED WITH DESTABILIZATION DOMAINS; U.S. application 62/096,697, 24 Dec. 14, CRISPR HAVING OR ASSOCIATED WITH AAV; U.S. application 62/098,158, 30 Dec. 14, ENGINEERED CRISPR COMPLEX INSERTIONAL TARGETING SYSTEMS; U.S. application 62/151,052, 22 Apr. 15, CELLULAR TARGETING FOR EXTRACELLULAR EXOSOMAL REPORTING; U.S. application 62/054,490, 24 Sep. 14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 14, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 14, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 14 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 14, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 14, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 14, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 14 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 14, FUNCTIONAL SCREENING WITH OPTIMIZED FUNCTIONAL CRISPR-CAS SYSTEMS; U.S. application 62/087,546, 4 Dec. 14 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 14, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

### Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of lHD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, R^{G}, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer, which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 2)
MDPIRSRTPSPARELLSGPQPDGVQPTADRGVSPPAGGPL
DGLPARRTMSRTRLPSPPAPSPAFSADSFSDLLRQFDPSLF
NTSLFDSLPPFGAHHTEAATGEWDEVQSGLRAADAPPPTM
RVAVTAARPPRAKPAPRRRAAQPSDASPAAQVDLRTLGY
SQQQQEKIKPKVRSTVAQHHEALVGHGFTHAHIVALSQHP
AALGTVAVKYQDMIAALPEATHEAIVGVGKQWSGARALE
ALLTVAGELRGPPLQLDTGQLLKIAKRGGVTAVEAVHAW
RNALTGAPLN

An exemplary amino acid sequence of a C-terminal capping region is:

(SEQ ID NO: 3)
RPALESIVAQLSRPDPALAALTNDHLVALACLGGRPALDA
VKKGLPHAPALIKRTNRRIPERTSHRVADHAQVVRVLGFF
QCHSHPAQAFDDAMTQFGMSRHGLLQLFRRVGVTELEAR
SGTLPPASQRWDRILQASGMKRAKPSPTSTQTPDQASLHA
FADSLERDLDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### ZN-Finger Nucleases

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et a1, Current Biology, 12, 735-739 (2002), Lagos Quintana et a1, Science 294, 853-857 (2001), and Lagos-Quintana et a1, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Inhibitors

In some embodiments, the modulating agent is an inhibitor that can inhibit one or more genes listed in any one of Tables 1-8 or any combination thereof or a gene product thereof.

As used herein, the terms “inhibitor,” “antagonist,” and “silencing agent,” refer to a molecule or agent that significantly blocks, inhibits, reduces, or interferes with one or more target genes or combinations, their biological activity in vitro, in situ, and/or in vivo, including activity of downstream pathways mediated by gene signaling. In some embodiments, the inhibitor or antagonist will modulate stromal cell markers. Exemplary inhibitors contemplated for use in the various aspects and embodiments described herein include, but are not limited to, antibodies or antigen-binding fragments thereof that specifically bind to one or more target genes listed in Table 1, Table 2, Table 3, Table 4, Table 5, Table 6, Table 7, and/or Table 8 or gene products thereof, or one or more subunits of the target gene(s)/product(s); anti-sense molecules directed to a nucleic acid encoding the target protein or subunits thereof; short interfering RNA (“siRNA”) molecules directed to a nucleic acid encoding the target protein or subunits thereof; RNA or DNA aptamers that bind to the target gene or gene product or a subunit thereof; gene product structural analog; soluble variant proteins or fusion polypeptides thereof; DNA targeting agents, such as CRISPR systems, Zinc finger binding proteins, TALES or TALENS; and small molecule agents that target or bind to the target gene or subunit(s) thereof. In some embodiments of the compositions, methods, and uses described herein, the inhibitor inhibits some or all of IL-27 mediated signal transduction. Exemplary assays to measure inhibition or reduction of downstream IL-27 signaling pathway activities are known to those of ordinary skill in the art and/or are provided herein.

As used herein, an inhibitor or antagonist has the ability to reduce the activity and/or expression of the target gene in a cell by at least 5%, at least 10%, at least 20%, at least 30%, at least 40%, at least 50%, at least 60%, at least 70%, at least 80%, at least 90%, at least 95%, at least 98%, at least 99%, or more, relative to the activity or expression level in the absence of the antagonist.

In some embodiments of the compositions, methods, and uses described herein, an inhibitor or antagonist is a monoclonal antibody.

In some embodiments of the compositions, methods, and uses described herein, an inhibitor or antagonist is an antibody fragment or antigen-binding fragment. The terms “antibody fragment,” “antigen binding fragment,” and “antibody derivative” as used herein, refer to a protein fragment that comprises only a portion of an intact antibody, generally including an antigen binding site of the intact antibody and thus retaining the ability to bind antigen.

In some embodiments of the compositions, methods, and uses described herein, an inhibitor or antagonist is a chimeric antibody derivative of an antagonist antibody or antigen binding fragment thereof.

The inhibitor or antagonist antibodies and antigen-binding fragments thereof described herein can also be, in some embodiments, a humanized antibody derivative.

In some embodiments, the inhibitor or antagonist antibodies and antigen-binding fragments thereof described herein, i.e., antibodies that are useful for decreasing T cell exhaustion, include derivatives that are modified, i.e., by the covalent attachment of any type of molecule to the antibody, provided that the covalent attachment does not prevent the antibody from binding to the target antigen

In some embodiments of the compositions, methods, and uses described herein, fully human antibodies are used, which are particularly desirable for the therapeutic treatment of human patients.

In some embodiments of the compositions, methods, and uses described herein, an inhibitor or antagonist is a small molecule inhibitor or antagonist, including, but is not limited to, small peptides or peptide-like molecules, soluble peptides, and synthetic non-peptidyl organic or inorganic compounds. A small molecule inhibitor or antagonist can have a molecular weight of any of about 100 to about 20,000 Daltons (Da), about 500 to about 15,000 Da, about 1000 to about 10,000 Da.

In some embodiments of the compositions, methods, and uses described herein, an inhibitor or antagonist is an RNA or DNA aptamer that binds or physically interacts with a target gene/gene product, and blocks interactions between the gene product and a binding partner.

In some embodiments of the compositions, methods, and uses described herein, an inhibitor or antagonist comprises at least one structural analog of a target gene/gene product as listed in Table 1, Table 2, Table 3, Table 4, Table 5, Table 6, Table 7, Table 8 or a combination thereof. The term “structural analogs” as used herein, refers to compounds that have a similar three-dimensional structure as the target gene or portion thereof, under physiological conditions in vitro or in vivo, wherein the binding of the analog in the signaling pathway reduces a desired biological activity. Suitable structural analogs can be designed and synthesized through molecular modeling of protein binding. The structural analogs and receptor structural analogs can be monomers, dimers, or higher order multimers in any desired combination of the same or different structures to obtain improved affinities and biological effects.

In some embodiments of the compositions, methods, and uses described herein, an inhibitor or antagonist comprises at least one soluble peptide, or portion of the target gene product, or fusion polypeptide thereof. In some such embodiments, the soluble peptide is fused to an immunoglobulin constant domain, such as an Fc domain, or to another polypeptide that modifies its in vivo half-life, e.g., albumin.

In some embodiments of the compositions, methods, and uses described herein, an inhibitor or antagonist comprises at least one antisense molecule capable of blocking or decreasing the expression of a desired target gene by targeting nucleic acids encoding the gene or subunit thereof. Methods are known to those of ordinary skill in the art for the preparation of antisense oligonucleotide molecules that will specifically bind one or more target gene(s) without cross-reacting with other polynucleotides. Exemplary sites of targeting include, but are not limited to, the initiation codon, the 5′ regulatory regions, including promoters or enhancers, the coding sequence, including any conserved consensus regions, and the 3′ untranslated region. In some embodiment of these aspects and all such aspects described herein, the antisense oligonucleotides are about 10 to about 100 nucleotides in length, about 15 to about 50 nucleotides in length, about 18 to about 25 nucleotides in length, or more. In certain embodiments, the oligonucleotides further comprise chemical modifications to increase nuclease resistance and the like, such as, for example, phosphorothioate linkages and 2′-O-sugar modifications known to those of ordinary skill in the art.

In some embodiments of the compositions, methods, and uses described herein, an inhibitor or antagonist comprises at least one siRNA molecule capable of blocking or decreasing the expression of a target gene product or a subunit thereof. Generally, one would prepare siRNA molecules that will specifically target one or more mRNAs without cross reacting with other polynucleotides. siRNA molecules for use in the compositions, methods, and uses described herein can be generated by methods known in the art, such as by typical solid phase oligonucleotide synthesis, and often will incorporate chemical modifications to increase half-life and/or efficacy of the siRNA agent, and/or to allow for a more robust delivery formulation. Alternatively, siRNA molecules are delivered using a vector encoding an expression cassette for intracellular transcription of siRNA. Other RNAi molecules that can be capable of acting as inhibitors are described elsewhere herein.

Inhibitors or antagonists for use in the compositions, methods, and uses described herein can be identified or characterized using methods known in the art, such as protein binding assays, biochemical screening assays, immunoassays, and cell-based assays, which are well known in the art.

### Activators

In some embodiments, the modulating agent can be an activator or an agonist. As used herein, the terms “activator,” “agonist,” or “activating agent,” refer to a molecule or agent that mimics or up-regulates (e.g., increases, potentiates or supplements) the expression and/or biological activity of a target gene/gene product in vitro, in situ, and/or in vivo, including downstream pathways mediated by gene signaling. For example, in some embodiments, an activator or agonist as described herein can modulate markers of a bone marrow stromal cell or population. An “activator” of a given polypeptide can include the polypeptide itself, in that supplying the polypeptide itself will increase the level of the function provided by the polypeptide. An activator or agonist can be a protein or derivative thereof having at least one bioactivity of the wild-type target gene/gene product. An activator or agonist can also be a compound that up-regulates expression of the desired target gene product or its subunits. An activator or agonist can also be a compound which increases the interaction of the target gene with its receptor, for example. Exemplary activators or agonists contemplated for use in the various aspects and embodiments described herein include, but are not limited to, antibodies or antigen-binding fragments thereof that specifically bind to a target gene/gene product or subunits thereof, RNA or DNA aptamers that bind to the target gene/gene product; structural analogs or soluble mimics or fusion polypeptides thereof; DNA targeting agents, such as CRISPR systems, Zinc finger binding proteins, and TALES; and small molecule agents that target or bind to a target gene product binding partner and act as functional mimics. Such molecules are described in greater detail elsewhere herein.

As used herein, an agonist has the ability to increase or enhance the activity and/or expression of a target gene/gene product in a cell by at least 5%, at least 10%, at least 20%, at least 30%, at least 40%, at least 50%, at least 60%, at least 70%, at least 80%, at least 90%, at least 95%, at least 98%, at least 99%, at least 100%, at least 1.5-fold, at least 2-fold, at least 5-fold, at least 10-fold, at least 25-fold, at least 50-fold, at least 100-fold, at least 1000-fold, or more relative to the activity or expression level in the absence of the activator or agonist.

In some embodiments of the compositions, methods, and uses described herein, the activator or agonist increases or enhances signal transduction mediated by the target gene/gene product. In some embodiments of the compositions and methods described herein, the activator or agonist increases or enhances transcription factor induction or activation.

In some embodiments of the compositions, methods, and uses described herein, the binding sites of the activators or agonists, such as an antibody or antigen-binding fragment thereof, are directed against an interaction site between the target gene product and one or more of its binding partners. By binding to an interaction site, an activator or agonist described herein can mimic or recapitulate the binding of the target gene product to its partner and increase the activity or expression of the target gene product, and downstream signaling consequences.

In some embodiments of the compositions, methods, and uses described herein, an activator or agonist is a monoclonal anti body. In some embodiments of the compositions, methods, and uses described herein, an activator or agonist is an antibody fragment or antigen-binding fragment.

In some embodiments of the compositions, methods, and uses described herein, an activator or agonist is a chimeric antibody derivative of the agonist antibodies and antigenbinding fragments thereof.

In some embodiments of the compositions, methods, and uses described herein, an activator or agonist is a humanized antibody derivative.

In some embodiments, the activator or agonist antibodies and antigen-binding fragments thereof described herein, i.e., antibodies that are useful for modulating bome marro cells and/or interactions with stem cells in the microenvironment, include derivatives that are modified, i.e., by the covalent attachment of any type of molecule to the antibody, provided that covalent attachment does not prevent the antibody from binding to the target antigen.

The activator or agonist antibodies and antigen-binding fragments thereof described herein can be generated by any suitable method known in the art.

In some embodiments, the activator or agonist antibodies and antigen-binding fragments thereof described herein are fully human antibodies or antigen-binding fragments thereof, which are particularly desirable for the therapeutic treatment of human patients. Human antibodies can be made by a variety of methods known in the art, and as described in more detail elsewhere herein.

In some embodiments of the compositions, methods, and uses described herein, an activator or agonist is a small molecule activator or agonist, including, but not limited to, small peptides or peptide-like molecules, soluble peptides, and synthetic non-peptidyl organic or inorganic compounds. A small molecule activator or agonist can have a molecular weight of any of about 100 to about 20,000 daltons (Da), about 500 to about 15,000 Da, or about 1000 to about 10,000 Da.

In some embodiments of the compositions, methods, and uses described herein, an activator or agonist is an RNA or DNA aptamer that binds or physically interacts with a target gene product and one or more of its binding partners, and enhances or promotes protein-protein interactions.

### Vectors

As described elsewhere herein, the stromal cells and other cells described herein can be engineered to express a gene of interest or a marker, or a modulating agent, or otherwise deliver a modulating agent to a cell, such as a stromal cell described herein. Such modulating agents and gene(s) can be delivered to a cell described herein via a vector or vector system. Suitable and exemplary vector systems are now described herein.

Thus, also provided herein are vectors that can contain one or more of the modulating agents and/or genes described herein. The vectors can be useful in producing bacterial, fungal, yeast, plant cells, animal cells, and transgenic animals that can express one or more modulating agents and/or genes described herein. Within the scope of this disclosure are vectors containing one or more of the polynucleotide sequences described herein. One or more of the modulating agents and/or genes described herein can be included in a vector or vector system. The vectors and/or vector systems can be used, for example, to express one or more of the polynucleotides in a cell, such as a producer cell, to produce viral particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g. a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other aspects of the vectors and vector systems are described elsewhere herein.

In some aspects, the vector can be a bicistronic vector. In some aspects, a bicistronic vector can be used for one or more modulating agents and/or genes described herein. In some aspects, expression of modulating agents and/or genes described herein can be driven by the CBh promoter. Where the element of the modulating agents and/or gene(s) is an RNA, its expression can be driven by a Pol III promoter, such as a U6 promoter. In some aspects, the two are combined.

### Cell-based Vector Amplification and Expression

Vectors can be designed for expression of one or more modulating agents and/or gene(s) described herein (e.g. nucleic acid transcripts, proteins, enzymes, and combinations thereof) in a suitable host cell. In some aspects, the suitable host cell is a prokaryotic cell. Suitable host cells include, but are not limited to, bacterial cells, yeast cells, insect cells, and mammalian cells. The vectors can be viral-based or non-viral based. In some aspects, the suitable host cell is a eukaryotic cell. In some aspects, the suitable host cell is a suitable bacterial cell. Suitable bacterial cells include, but are not limited to bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some aspects, the host cell is a suitable insect cell. Suitable insect cells include those from Spodoptera frugiperda. Suitable strains of S. frugiperda cells include, but are not limited to Sf9 and Sf21. In some aspects, the host cell is a suitable yeast cell. In some aspects, the yeast cell can be from Saccharomyces cerevisiae. In some aspects, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U20S, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some aspects, the vector can be a yeast expression vector. Examples of vectors for expression in yeast Saccharomyces cerevisiae include pYepSecl (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some aspects, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some aspects, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other aspects can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more modulating agents and/or gene(s) so as to drive expression of the one or more modulating agents and/or gene(s) described herein.

Vectors may be introduced and propagated in a prokaryote or prokaryotic cell. In some aspects, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some aspects, a prokaryote is used to amplify copies of a vector and express one or more nucleic acids, such as to provide a source of one or more proteins for delivery to a host cell or host organism.

In some aspects, the vector can be a fusion vector or fusion expression vector. In some aspects, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some aspects, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some aspects, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more modulating agents and/or gene(s) described herein are introduced into a host cell such that expression of the modulating agents and/or gene(s) described herein direct formation of a modulating agent and/or gene delivery system described herein (including but not limited to a virus particle, which is described in greater detail elsewhere herein). For example, different modulating agents and/or gene(s) described herein can each be operably linked to separate regulatory elements on separate vectors. RNA(s) of different modulating agents and/or gene(s) described herein can be delivered to an animal or mammal or cell thereof to produce an animal or mammal or cell thereof that constitutively or inducibly or conditionally expresses different modulating agents and/or gene(s) described herein that incorporates one or more modulating agents and/or gene(s) described herein or contains one or more cells that incorporates and/or expresses one or more modulating agents and/or gene(s) described herein.

In some aspects, two or more of the elements expressed from the same or different regulatory element(s), can be combined in a single vector, with one or more additional vectors providing any components of the system not included in the first vector. Modulating agents and/or gene(s) that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more modulating agents and/or gene(s), embedded within one or more intron sequences (e.g., each in a different intron, two or more in at least one intron, or all in a single intron). In some embodiments, the one or more modulating agents and/or gene(s) can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g. molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In aspects, the polynucleotides and/or vectors thereof described herein can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et a1, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some aspects, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some aspects, the vector can contain a minimal promoter. In some aspects, the minimal promoter is the Mecp2 promoter, tRNA promoter, or U6. In a further embodiment, the minimal promoter is tissue specific. In some aspects, the length of the vector polynucleotide the minimal promoters and polynucleotide sequences is less than 4.4Kb.

To express a polynucleotide, the vector can include one or more transcriptional and/or translational initiation regulatory sequences, e.g. promoters, that direct the transcription of the gene and/or translation of the encoded protein in a cell. In some aspects a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some aspects, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some aspects, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdxl, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Ferl14), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the modulating agents and/or gene(s) described herein are typically placed under control of a plant promoter, i.e. a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the modulating agents and/or gene(s) describe herein are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the vectors described herein are found in Kawamata et al., (1997) Plant Cell Physiol 38:792-803; Yamamoto et al., (1997) Plant J 12:255-65; Hire et a1, (1992) Plant Mol Biol 20:207-18, Kuster et a1, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more modulating agents and/or gene(s) described herein, a light-responsive cytochrome heterodimer (e.g. from Arabidopsis thaliana), and a transcriptional activation/repression domain. In some aspects, the vector can include one or more of the inducible DNA binding proteins provided in PCT publication WO 2014/018423 and US Publications, 2015/0291966, 2017/0166903, 2019/0203212, which describe e.g. aspects of inducible DNA binding proteins and methods of use and can be adapted for use with the present invention.

In some aspects, transient or inducible expression can be achieved by including, for example, chemical-regulated promotors, i.e. whereby the application of an exogenous chemical induces gene expression. Modulation of gene expression can also be obtained by including a chemical-repressible promoter, where application of the chemical represses gene expression. Chemical-inducible promoters include, but are not limited to, the maize ln 2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some aspects, the vector or system thereof can include one or more elements capable of translocating and/or expressing one or more modulating agents and/or gene(s) described herein to/in a specific cell component or organelle. Such organelles can include, but are not limited to, nucleus, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

One or more of the modulating agents and/or gene(s) can be can be operably linked, fused to, or otherwise modified to include a polynucleotide that encodes or is a selectable marker or tag, which can be a polynucleotide or polypeptide. In some aspects, the polynucleotide encoding a polypeptide selectable marker can be incorporated in the vector capable of expressing one or more modulating agents and/or genes such that the selectable marker polynucleotide, when translated, is inserted between two amino acids between the N- and C-terminus of the modulating agent(s) and/or gene product(s) polypeptide(s) and/or at the N- and/or C-terminus of the modulating agent(s) and/or gene product(s) polypeptide(s). In some aspects, the selectable marker or tag is a polynucleotide barcode or unique molecular identifier (UMI).

It will be appreciated that the polynucleotide encoding such selectable markers or tags can be incorporated into a polynucleotide encoding one or more modulating agents and/or gene product(s) described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with F1AsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as 3-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more modulating agents and/or gene product(s) described herein via suitable linker, such as a glycine or glycine serine linkers as short as GS or GG up to (GGGGG)₃ (SEQ ID NO: 4) or (GGGGS)₃. (SEQ ID NO: 5) Other suitable linkers are described elsewhere herein.

The vector or vector system can include one or more polynucleotides encoding one or more targeting moieties. In some aspects, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some aspects, the targeting moiety encoding polynucleotides can be included in the vector or vector system such that the modulating agents and/or gene(s) and/or products expressed therefrom include the targeting moiety and can be targeted to specific cells, tissues, organs, etc. In some aspects, such as non-viral carriers, the targeting moiety can be attached to the carrier (e.g. polymer, lipid, inorganic molecule etc.) and can be capable of targeting the carrier and any attached or associated modulating agents and/or gene(s) polynucleotide(s) to specific cells, tissues, organs, etc.

### Cell-Free Vector and Polynucleotide Expression

In some aspects, the polynucleotide encoding one or more modulating agents and/or gene product(s) described herein can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g. translation of a purified polyribonucleotide) or linked/coupled to transcription. In some aspects, the cell-free (or in vitro) translation system can include extracts from rabbit reticulocytes, wheat germ, and/or E. coli. The extracts can include various macromolecular components that are needed for translation of exogenous RNA (e.g. 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g. reticulocyte lysates and wheat germ extracts). Some translation systems can utilize a DNA template as a starting material (e.g. E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding one or more modulating agents and/or gene product (s) described herein can be codon optimized. In some aspects, one or more polynucleotides contained in a vector (“vector polynucleotides”) described herein that are in addition to an optionally codon optimized polynucleotide encoding modulating agents and/or gene product (s) described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et a1, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some aspects, a codon optimized sequence is a sequence optimized for expression in a eukaryote, e.g., humans (i.e. being optimized for expression in a human or human cell), or for another eukaryote, such as another animal (e.g. a mammal or avian) as is described elsewhere herein. Such codon optimized sequences are within the ambit of the ordinary skilled artisan in view of the description herein. In some aspects, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g. cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some aspects, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some aspects, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some aspects, the vector is a non-viral vector or carrier. In some aspects, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with an modulating agents and/or gene(s) of the present invention and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid or polynucleotide molecule or composition that be attached to or otherwise interact with a polynucleotide to be delivered, such as modulating agents and/or gene(s), and/or gene products of the present invention.

### Naked Polynucleotides

In some aspects one or more modulating agents and/or gene(s) described elsewhere herein can be included in a naked polynucleotide. The term of art “naked polynucleotide” as used herein refers to polynucleotides that are not associated with another molecule (e.g. proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the modulating agents and/or gene(s) described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g. plasmids and artificial chromosomes), molecules that contain portions that are single stranded and portions that are double stranded (e.g. ribozymes), and the like. In some aspects, the naked polynucleotide contains only the modulating agents and/or gene(s) of the present invention. In some aspects, the naked polynucleotide can contain other nucleic acids and/or polynucleotides in addition to the modulating agents and/or gene(s) of the present invention. The naked polynucleotides can include one or more elements of a transposon system. Transposons and system thereof are described in greater detail elsewhere herein.

### Non-Viral Polynucleotide Vectors

In some aspects, one or more of the modulating agents and/or gene(s) can be included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g. minicircles, minivectors, miniknots), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g. Hardee et al. 2017. Genes. 8(2):65.

In some aspects, the non-viral polynucleotide vector can have a conditional origin of replication. In some aspects, the non-viral polynucleotide vector can be an ORT plasmid. In some aspects, the non-viral polynucleotide vector can have a minimalistic immunologically defined gene expression. In some aspects, the non-viral polynucleotide vector can have one or more post-segregationally killing system genes. In some aspects, the non-viral polynucleotide vector is AR-free. In some aspects, the non-viral polynucleotide vector is a minivector. In some aspects, the non-viral polynucleotide vector includes a nuclear localization signal. In some aspects, the non-viral polynucleotide vector can include one or more CpG motifs. In some aspects, the non-viral polynucleotide vectors can include one or more scaffold/matrix attachment regions (S/MARs). See e.g. Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In aspects, the S/MAR sequence is located downstream of an actively transcribed polynucleotide (e.g. one or more modulating agents and/or gene(s) of the present invention) included in the non-viral polynucleotide vector. In some aspects, the S/MAR can be a S/MAR from the beta-interferon gene cluster. See e.g. Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some aspects, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some aspects, the non-viral polynucleotide vector can be a retrotransposon vector. In some aspects, the retrotransposon vector includes long terminal repeats. In some aspects, the retrotransposon vector does not include long terminal repeats. In some aspects, the non-viral polynucleotide vector can be a DNA transposon vector. DNA transposon vectors can include a polynucleotide sequence encoding a transposase. In some aspects, the transposon vector is configured as a non-autonomous transposon vector, meaning that the transposition does not occur spontaneously on its own. In some of these aspects, the transposon vector lacks one or more polynucleotide sequences encoding proteins required for transposition. In some aspects, the non-autonomous transposon vectors lack one or more Ac elements.

In some aspects a non-viral polynucleotide transposon vector system can include a first polynucleotide vector that contains the modulating agents and/or gene(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g. the modulating agents and/or gene(s) of the present invention) and integrate it into one or more positions in the host cell's genome. In some aspects the transposon vector or system thereof can be configured as a gene trap. In some aspects, the TIRs can be configured to flank a strong splice acceptor site followed by a reporter and/or other gene (e.g. one or more of modulating agents and/or gene(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, Sleeping Beauty transposon system (Tc1/mariner superfamily) (see e.g. Ivics et al. 1997. Cell. 91(4): 501-510), piggyBac (piggyBac superfamily) (see e.g. Li et al. 2013 110(25): E2279-E2287 and Yusa et al. 2011. PNAS. 108(4): 1531-1536), To12 (superfamily hAT), Frog Prince (Tc1/mariner superfamily) (see e.g. Miskey et al. 2003 Nucleic Acid Res. 31(23):6873-6881) and variants thereof.

### Chemical Carriers

In some aspects the modulating agents and/or gene(s) can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the modulating agents and/or gene(s) of the present invention), (2) those capable of targeting specific cells, (3) those capable of increasing delivery of the polynucleotide (such as the modulating agents and/or gene(s) of the present invention) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the modulating agents and/or gene(s)/and/or gene products described herein. Suitable sizes include macro-, micro-, and nano-sized particles.

In some aspects, the non-viral carrier can be an inorganic particle. In some aspects, the inorganic particle, can be a nanoparticle. The inorganic particles can be configured and optimized by varying size, shape, and/or porosity. In some aspects, the inorganic particles are optimized to escape from the reticulo endothelial system. In some aspects, the inorganic particles can be optimized to protect an entrapped molecule from degredation, the Suitable inorganic particles that can be used as non-viral carriers in this context can include, but are not limited to, calcium phosphate, silica, metals (e.g. gold, platinum, silver, palladium, rhodium, osmium, iridium, ruthenium, mercury, copper, rhenium, titanium, niobium, tantalum, and combinations thereof), magnetic compounds, poarticles, and materials, (e.g. supermagnetic iron oxide and magnetite), quantum dots, fullerenes (e.g. carbon nanoparticles, nanotubes, nanostrings, and the like), and combinations thereof. Other suitable inorganic non-viral carriers are discussed elsewhere herein.

In some aspects, the non-viral carrier can be lipid-based. Suitable lipid-based carriers are also described in greater detail herein. In some aspects, the lipid-based carrier includes a cationic lipid or an amphiphilic lipid that is capable of binding or otherwise interacting with a negative charge on the polynucleotide to be delivered (e.g. such as an modulating agents and/or gene(s) of the present invention). In some aspects, chemical non-viral carrier systems can include a polynucleotide such as the modulating agents and/or gene(s) of the present invention) and a lipid (such as a cationic lipid). These are also referred to in the art as lipoplexes. Other aspects of lipoplexes are described elsewhere herein. In some aspects, the non-viral lipid-based carrier can be a lipid nano emulsion. Lipid nano emulsions can be formed by the dispersion of an immisicible liquid in another stabilized emulsifying agent and can have particles of about 200 nm that are composed of the lipid, water, and surfactant that can contain the polynucleotide to be delivered (e.g. the modulating agents and/or gene(s) of the present invention). In some aspects, the lipid-based non-viral carrier can be a solid lipid particle or nanoparticle.

In some aspects, the non-viral carrier can be peptide-based. In some aspects, the peptide-based non-viral carrier can include one or more cationic amino acids. In some aspects, 35 to 40, 45, 50, 55, 60, 65, 70, 75, 80, 85, 90, 95, 99 or 100% of the amino acids are cationic. In some aspects, peptide carriers can be used in conjunction with other types of carriers (e.g. polymer-based carriers and lipid-based carriers to functionalize these carriers). In some aspects, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g. US Pat. Pub. 2017/0079916 whose techniques and compositions can be adapted for use with the modulating agents and/or gene(s) of the present invention), polymethacrylate, and combinations thereof.

In some aspects, the non-viral carrier can be configured to release modulating agents and/or gene(s) that is associated with or attached to the non-viral carrier in response to an external stimulus, such as pH, temperature, osmolarity, concentration of a specific molecule or composition (e.g. calcium, NaCl, and the like), pressure and the like. In some aspects, the non-viral carrier can be a particle that is configured includes one or more of the modulating agents and/or gene(s) described herein and a environmental triggering agent response element, and optionally a triggering agent. In some aspects, the particle can include a polymer that can be selected from the group of polymethacrylates and polyacrylates. In some aspects, the non-viral particle can include one or more aspects of the compositions microparticles described in US Pat. Pubs. 20150232883 and 20050123596, whose techniques and compositions can be adapted for use in the present invention.

In some aspects, the non-viral carrier can be a polymer-based carrier. In some aspects, the polymer is cationic or is predominantly cationic such that it can interact in a charge-dependent manner with the negatively charged polynucleotide to be delivered (such as the modulating agents and/or gene(s) of the present invention). Polymer-based systems are described in greater detail elsewhere herein.

### Viral Vectors

In some aspects, the vector is a viral vector. The term of art “viral vector” and as used herein in this context refers to polynucleotide based vectors that contain one or more elements from or based upon one or more elements of a virus that can be capable of expressing and packaging a polynucleotide, such as an modulating agents and/or gene(s) of the present invention, into a virus particle and producing said virus particle when used alone or with one or more other viral vectors (such as in a viral vector system). Viral vectors and systems thereof can be used for producing viral particles for delivery of and/or expression of one or more modulating agents and/or gene(s) described herein. The viral vector can be part of a viral vector system involving multiple vectors. In some aspects, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other aspects of viral vectors and viral particles produce therefrom are described elsewhere herein. In some aspects, the viral vectors are configured to produce replication incompetent viral particles for improved safety of these systems.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the modulating agents and/or gene(s) can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some aspects, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the modulating agents and/or gene(s) described herein.

In some aspects, the lentiviral vector or vector system thereof can be a first-generation lentiviral vector or vector system thereof. First-generation lentiviral vectors can contain a large portion of the lentivirus genome, including the gag and pol genes, other additional viral proteins (e.g. VSV-G) and other accessory genes (e.g. vif, vprm vpu, nef, and combinations thereof), regulatory genes (e.g. tat and/or rev) as well as the gene of interest between the LTRs. First generation lentiviral vectors can result in the production of virus particles that can be capable of replication in vivo, which may not be appropriate for some instances or applications.

In some aspects, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some aspects, the second-generation vector lacks one or more accessory virulence factors (e.g. vif, vprm, vpu, nef, and combinations thereof). Unlike the first-generation lentiviral vectors, no single second generation lentiviral vector includes all features necessary to express and package a polynucleotide into a virus particle. In some aspects, the envelope and packaging components are split between two different vectors with the gag, pol, rev, and tat genes being contained on one vector and the envelope protein (e.g. VSV-G) are contained on a second vector. The gene of interest, its promoter, and LTRs can be included on a third vector that can be used in conjunction with the other two vectors (packaging and envelope vectors) to generate a replication-incompetent virus particle.

In some aspects, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In some aspects, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g. gag, pol, and rev) and upstream regulatory sequences (e.g. promoter(s)) to drive expression of the features present on the packaging vector, and (iii) an “envelope vector” that contains one or more envelope protein genes and upstream promoters. In aspects, the third-generation lentiviral vector system can include at least two packaging vectors, with the gag-pol being present on a different vector than the rev gene.

In some aspects, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the modulating agents and/or gene(s) of the present invention.

In some aspects, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some aspects, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD114) (see e.g. Hanawa et al. Molec. Ther. 2002 5(3) 242-251), modified Sindbis virus envelope proteins (see e.g. Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g. Girard-Gagnepain et al. 2014. Blood. 124: 1221-1231); Tupaia paramyxovirus glycoproteins (see e.g. Enkirch T. et al., 2013. Gene Ther. 20:16-23); measles virus glycoproteins (see e.g. Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gpl20 of HIV, hemagglutinin, neuraminidase, M2 proteins of influenza virus, and combinations thereof.

In some aspects, the tropism of the resulting lentiviral particle can be tuned by incorporating cell targeting peptides into a lentiviral vector such that the cell targeting peptides are expressed on the surface of the resulting lentiviral particle. In some aspects, a lentiviral vector can contain an envelope protein that is fused to a cell targeting protein (see e.g. Buchholz et al. 2015. Trends Biotechnol. 33:777-790; Bender et al. 2016. PLoS Pathog. 12(e1005461); and Friedrich et a1. 2013. Mol. Ther. 2013. 21: 849-859.

In some aspects, a split-intein-mediated approach to target lentiviral particles to a specific cell type can be used (see e.g. Chamoun-Emaneulli et al. 2015. Biotechnol. Bioeng. 112:2611-2617, Ramirez et al. 2013. Protein. Eng. Des. Sel. 26:215-233. In these aspects, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some aspects, a covalent-bond-forming protein-peptide pair can be incorporated into one or more of the lentiviral vectors described herein to conjugate a cell targeting peptide to the virus particle (see e.g. Kasaraneni et al. 2018. Sci. Reports (8) No. 10990). In some aspects, a lentiviral vector can include an N-termial PDZ domain of InaD protein (PDZ1) and its pentapeptide ligand (TEFCA) from NorpA, which can conjugate the cell targeting peptide to the virus particle via a covalent bond (e.g. a disulfide bond). In some aspects, the PDZ1 protein can be fused to an envelope protein, which can optionally be binding deficient and/or fusion competent virus envelope protein and included in a lentiviral vector. In some aspects, the TEFCA can be fused to a cell targeting peptide and the TEFCA-CPT fusion construct can be incorporated into the same or a different lentiviral vector as the PDZl-envenlope protein construct. During virus production, specific interaction between the PDZ 1 and TEFCA facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver one or more modulating agents and/or gene(s) described herein to a cell.

In some aspects, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g. antibiotic resistance genes), Psi (ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

### Adenoviral Vectors, Helper-Dependent Adenoviral Vectors, and Hybrid Adenoviral Vectors

In some aspects, the vector can be an adenoviral vector. In some aspects, the adenoviral vector can include elements such that the virus particle produced using the vector or system thereof can be serotype 2 or serotype 5. In some aspects, the polynucleotide to be delivered via the adenoviral particle can be up to about 8 kb. Thus, in some aspects, an adenoviral vector can include a DNA polynucleotide to be delivered that can range in size from about 0.001 kb to about 8 kb. Adenoviral vectors have been used successfully in several contexts (see e.g. Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some aspects the vector can be a helper-dependent adenoviral vector or system thereof. These are also referred to in the art as “gutless” or “gutted” vectors and are a modified generation of adenoviral vectors (see e.g. Thrasher et al. 2006. Nature. 443:E5-7). In aspects of the helper-dependent adenoviral vector system one vector (the helper) can contain all the viral genes required for replication but contains a conditional gene defect in the packaging domain. The second vector of the system can contain only the ends of the viral genome, one or more modulating agents and/or gene(s), and the native packaging recognition signal, which can allow selective packaged release from the cells (see e.g. Cideciyan et al. 2009. N Engl J Med. 361:725-727). Helper-dependent adenoviral vector systems have been successful for gene delivery in several contexts (see e.g. Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the modulating agents and/or gene(s) described herein. In some aspects, the polynucleotide to be delivered via the viral particle produced from a helper-dependent adenoviral vector or system thereof can be up to about 37 kb. Thus, in some aspects, a adenoviral vector can include a DNA polynucleotide to be delivered that can range in size from about 0.001 kb to about 37 kb (see e.g. Rosewell et al. 2011. J. Genet. Syndr. Gene Ther. Suppl. 5:001).

In some aspects, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some aspects, such hybrid vector systems can result in stable transduction and limited integration site. See e.g. Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use with the modulating agents and/or gene(s) of the present invention. In some aspects, a hybrid-adenoviral vector can include one or more features of a retrovirus and/or an adeno-associated virus. In some aspects the hybrid-adenoviral vector can include one or more features of a spuma retrovirus or foamy virus (FV). See e.g. Ehrhardt et al. 2007. Mol. Ther. 15:146-156 and Liu et al. 2007. Mol. Ther. 15:1834-1841, whose techniques and vectors described therein can be modified and adapted for use with the modulating agents and/or gene(s) of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g. Ehrhardt et al. 2007. Mol. Ther. 156:146-156 and Shuji et al. 2011. Mol. Ther. 19:76-82, whose techniques and vectors described therein can be modified and adapted for use with the modulating agents and/or gene(s) of the present invention.

### Adeno Associated Viral (AA V) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some aspects the AAV can integrate into a specific site on chromosome 19 of a human cell with no observable side effects. In some aspects, the capacity of the AAV vector, system thereof, and/or AAV particles can be up to about 4.7 kb.

The AAV vector or system thereof can include one or more regulatory molecules. In some aspects the regulatory molecules can be promoters, enhancers, repressors and the like, which are described in greater detail elsewhere herein. In some aspects, the AAV vector or system thereof can include one or more polynucleotides that can encode one or more regulatory proteins. In some aspects, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some aspects, the AAV capsid can contain 60 capsid proteins. In some aspects, the ratio of VP1:VP2:VP3 in a capsid can be about 1:1:10.

In some aspects, the AAV vector or system thereof can include one or more adenovirus helper factors or polynucleotides that can encode one or more adenovirus helper factors. Such adenovirus helper factors can include, but are not limited, E1A, E1B, E2A, E4ORF6, and VA RNAs. In some aspects, a producing host cell line expresses one or more of the adenovirus helper factors.

The AAV vector or system thereof can be configured to produce AAV particles having a specific serotype. In some aspects, the serotype can be AAV-1, AAV-2, AAV-3, AAV-4, AAV-5, AAV-6, AAV-8, AAV-9 or any combinations thereof. In some aspects, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some aspects, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some aspects, an AAV vector or system thereof capable of producing AAV particles capable of targeting cardiac tissue can be configured to generate an AAV particle having an AAV-4 serotype. In some aspects, an AAV vector or system thereof capable of producing AAV particles capable of targeting the liver can be configured to generate an AAV having an AAV-8 serotype. In some aspects, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et a1, J. Virol. 82: 5887-5911 (2008) as recapitulated below in Table 9.

TABLE 9

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

In some aspects, the AAV vector or system thereof is configured as a “gutless” vector, similar to that described in connection with a retroviral vector. In some aspects, the “gutless” AAV vector or system thereof can have the cis-acting viral DNA elements involved in genome amplification and packaging in linkage with the heterologous sequences of interest (e.g. the modulating agents and/or gene(s) described herein).

### Herpes Simplex Viral Vectors

In some aspects, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g. 2009. Trobridge. Exp. Opin. Biol. Ther. 9:1427-1436, whose techniques and vectors described therein can be modified and adapted for use with the modulating agents and/or gene(s) of the present invention. In some aspects where an HSV vector or system thereof is utilized, the host cell can be a complementing cell. In some aspects, HSV vector or system thereof can be capable of producing virus particles capable of delivering a polynucleotide cargo of up to 150 kb. Thus, in some aspects the modulating agents and/or gene(s) included in the HSV-based viral vector or system thereof can sum from about 0.001 to about 150 kb. HSV-based vectors and systems thereof have been successfully used in several contexts including various models of neurologic disorders. See e.g. Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use with the modulating agents and/or gene(s) of the present invention.

### Poxvirus Vectors

In some aspects, the vector can be a poxvirus vector or system thereof. In some aspects, the poxvirus vector can result in cytoplasmic expression of one or more modulating agents and/or gene(s) of the present invention. In some aspects the capacity of a poxvirus vector or system thereof can be about 25 kb or more. In some aspects, a poxivirus vector or system thereof can include a

### Vector Construction

The vectors described herein can be constructed using any suitable process or technique. In some aspects, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more modulating agents and/or gene(s) described herein are as used in the foregoing documents, such as WO 2014/093622 (PCT/US2013/074667) and are discussed in greater detail herein.

Virus Particle Production from Viral Vectors

### Retroviral Production

In some aspects, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some aspects, the suitable host cell for virus production from viral vectors and systems thereof described herein can stably express one or more genes involved in packaging (e.g. pol, gag, and/or VSV-G) and/or other supporting genes.

In some aspects, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g. one or more modulating agents and/or gene(s)), and virus particle assembly, and secretion of mature virus particles into the culture media. Various other methods and techniques are generally known to those of ordinary skill in the art.

Mature virus particles can be collected from the culture media by a suitable method. In some aspects, this can involve centrifugation to concentrate the virus. The titer of the composition containing the collected virus particles can be obtained using a suitable method. Such methods can include transducing a suitable cell line (e.g. NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some aspects, the resulting composition containing virus particles can contain 1×10¹-1×10²⁰ particles/mL.

### AAV Particle Production

There are two main strategies for producing AAV particles from AAV vectors and systems thereof, such as those described herein, which depend on how the adenovirus helper factors are provided (helper v. helper free). In some aspects, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g. the modulating agents and/or gene(s)). In some aspects, a method of producing AAV particles from AAV vectors and systems thereof can be a “helper free” method, which includes co-transfection of an appropriate producing cell line with three vectors (e.g. plasmid vectors): (1) an AAV vector that contains a polynucleotide of interest (e.g. the modulating agents and/or gene(s)) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., modulating agents and/or gene(s) transcripts, proteins, enzymes, mutant forms thereof, fusion proteins thereof, etc.), and virus particles (such as from viral vectors and systems thereof).

One or more modulating agents and/or gene(s) can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some aspects, doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered in to a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g. injections), ballistic polynucleotides (e.g. particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of Modulating Agents and/or Gene(s) to Cells Via Particles.

The term “particle” as used herein, refers to any suitable sized particles for delivery of the modulating agents and/or gene(s) described herein. Suitable sizes include macro-, micro-, and nano-sized particles. In some aspects, any of the of the modulating agents and/or gene(s) (e.g. polypeptides, polynucleotides, vectors and combinations thereof described herein) can be attached to, coupled to, integrated with, otherwise associated with one or more particles or component thereof as described herein. The particles described herein can then be administered to a cell or organism by an appropriate route and/or technique. In some aspects, particle delivery can be selected and be advantageous for delivery of the polynucleotide or vector components. It will be appreciated that in aspects, particle delivery can also be advantageous for other modulating agents and/or gene(s), molecules, and formulations described elsewhere herein.

### Methods of Screening for Bone Marrow Stromal Cell Modulating Agents

In an embodiment, a method of identifying a candidate agent that modulates a stromal cell type or a stromal cell subtype, or a population of stromal cells or subtype, comprises contacting the cell, subtype, or population thereof with a test compound, obtaining gene expression information from the cell, subtype, or population thereof and comparing with a suitable control. In certain embodiments, a suitable control is cell, subtype, or population thereof that has not been contacted with the test compound. In certain embodiments, a suitable control is a corresponding cell, subtype, or population thereof that is dysfunctional.

Described herein are methods of identifying agents that modulate characteristics of bone marrow stromal cells. Also provided are methods of identifying agents that target interactions of cancer cells with bone marrow stromal cells. In general, the screening methods include contacting a test compound with a test sample. The test samples used in the screening methods described herein include cultures of bone marrow stromal cells and stromal cell subsets, co-cultures with cancer cells, e.g., primary cancer cells, e.g., primary hematopoietic cells, e.g., leukemic cells (preferably enriched for LSCs) and/or normal cells (preferably enriched for HSCs and progenitor cells). In some embodiments the test samples include both LSCs and normal cells (e.g., HPSCs) commingled together in a “triple coculture,” which is useful for a side-by side comparison of effects and crosstalk effects. The test samples may be provided from animal models, of from sources suitable for high-throughput screening, including but not limited to multi-well plate or culture dish, three-dimensional culture such as but not limited to 3D gels and organoids.

The primary hematopoietic cells are preferably enriched for stem and progenitor cells, are preferably mammalian, and can be obtained using methods known in the art. For example, the primary hematopoietic cells can be obtained from the bone marrow of a rodent, e.g., a mouse or rat, or other experimental animal. Alternatively, the primary hematopoietic cells can be human in origin, e.g., obtained from a bone marrow aspiration, e.g., from a subject. In some embodiments, the primary hematopoietic cells can be genetically engineered to express a detectable marker, such as a fluorescent protein (e.g., green fluorescent protein or a variant thereof as known in the art) that allows identification or measurement of a cell or component. Genetic engineering of cells is described elsewhere herein.

The stromal cells useful in the test samples can include primary and/or cultured stromal cells. Stromal cells are any non-parenchymal cells, also referred to as connective tissue cells, and are typically adherent when bone marrow is grown in culture. They constitute the non-blood forming fraction of bone marrow, and are sometimes referred to also as mesenchymal stromal cells or multipotent mesenchymal stromal cells (Brinchmann, 2008). Such cells have the potential to differentiate into various stromal cell types, such as osteoblasts and adipocytes. Other examples of stromal cell types include endothelial and perivascular cells. Stromal cell lines include lxN/2b; AC6.21; AFT024; AGM-S3; FLS4.1; FS-1; HAS303; HCB1-SV40; HESS-5; HM1-SV40; HM2-SV40; HYMEQ-5; KM102; L87/4; MRL104.8a; MS-5; OP9; PA6; PK-2; PU-34; S10; SI 7; S21; Saka; SCLl-24; SC-MSC; SPY3-2; SR-4987; SSL 1; ST-1; ST2; and TBR59 cell lines. In some embodiments, the stromal cells and the primary hematopoietic cells are from the same species. In some embodiments, the stromal cells are also genetically engineered to express a detectable marker that allows detection or measurement of a cell or component.

Described herein are methods for determining the suitability of a compound for modulating a dysfunctional stromal phenotype and/or modulating a stromal response, said method comprising contacting a stromal cell expressing the signature of dysfunction as defined above with said compound and determining whether or not said compound can affect the expression of the signature by said cell.

In some exemplary embodiments, described herein are methods of screening for one or more agents capable of modulating a stromal cell state, comprising: contacting a stromal cell population having an initial cell state with a test modulating agent or library of modulating agents, wherein the stromal cell population optionally contains leukemia cells; determining one or more fractions of stromal cell states including one or more fraction(s) of a mesenchymal stem/stromal cell (MSC), an OLC, a chondrocyte, a fibroblast, a pericyte, a bone marrow derived endothelial cell (BMEC), or a combination thereof; and selecting modulating agents that shifts the initial stromal cell state to a desired stromal cell state, wherein the desired stromal cell fraction in the stromal cell population is above a set cutoff limit. In some exemplary embodiments, determining one or more fractions of stromal cell states further comprises determining one or more MSC subtype, one or more OLC types, one or more chondrocyte types, one or more fibroblast types, one or more BMEC types, one or more pericyte subtype, or a combination thereof. In some exemplary embodiments, the stromal cell population is obtained from a subject to be treated. In some exemplary embodiments, determining one or more fractions of stromal cell states comprises identifying a MSC gene signature, an OLC gene signature, a chondrocyte gene signature, a fibroblast gene signature, a BMEC gene signature, a pericyte gene signature.

In some exemplary embodiments, the MSC gene signature comprises:

a. one or more genes of Table 1;

b. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or

c. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxcl1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1;

and wherein the MCS optionally does not express one or more of Thy1, Ly6a (Sca-1), NG2 (Cspg4) or Nestin (Nes).

In some exemplary embodiments, the OLC gene signature comprises:

a. one or more genes of Table 2;

b. one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;

c. one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmeml19, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbp11, Col3a1, Ptn, Col6a2, Tnn, Npy, Col6a1, Omd, Dcn, Tgfbi, Col6a3, or Acan;

d. one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13;

and wherein the OLC optionally expresses Bglap and Spp1.

In some exemplary embodiments, the chondrocyte gene signature comprises:

a. one or more genes of Table 4;

b. one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst11, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, Il17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;

c. one or more of Sox9, Col11a2, Acan, or Col2a1;

d. one or more of Runx2, Ihh, Mef2c, or Col10a1;

e. one or more of Grem1, Runx2, Sp7, Alp1, or Spp1;

f. one or more of Ihh, Pth1r, Mef2c, Col10a1, Ibsp, Mmp13, Grem 1; or

g. one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, Il17b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

the fibroblast gene signature comprises:

a. one or more genes of Table 5;

b. one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;

c. Fibronectin-1 (Fn1), Fibroblast Specific Protein-1 (S100a4), Col1a1, Col1a2, Lum, Col22a1, or Twist2;

d. one or more of Sox9, Acan, and Col2a1;

e. Cd34, Ly6a, Pdgfra, Thy1 and Cd44, and not Cdh5, or Acta2;

f. one or more of Sox-9, Scleraxis (Scx), Spp1, Cspg4, CD73 (Nt5e), and Cartilage Intermediate Layer Protein (Cilp); or

g. one or more of S1004a, Dcn, Sema3c, or Cxcl12.

In some exemplary embodiments, the the BMEC gene signature comprises:

a. one or more genes of Table 6;

b. one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x;

c. one or more of Flt4 (Vegfr-3) or Ly6a (Sca-1);

d. one or more of Pecam1, Cdh5, Cd34, Tek, Lepr, Cxcl12, or Kitl;

e. one or more of Flt4, Ly6a, Icam1, or Sele;

f. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, lfitm2; or

g. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

In some exemplary embodiments, the pericyte gene signature comprises:

a. one or more genes in Table 3;

b. one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, Cygp;

c. one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11; or

d. one or more of Acta2, Myh11, Mcam, Jag1, or Il6.

In some exemplary embodiments, the modulating agent that shifts the initial stromal cell state to the desired stromal cell state is capable of remodeling in a hematological disease.

In some exemplary embodiments, described herein are methods of screening for one or more agents capable of modulating osteogenic and/or adipogenic differentiation in a hematological disease comprising: contacting a cell population with a test modulating agent, wherein the cell population comprises MSC(s), OLC(s), and leukemia cells; and selecting modulating agents that change the regulation of one or more of Grem1, Bmp4, Sp7, Runx2, Bglap1, Bglap2, Bglap3, Adipoq, Wisp2, Mgp, Igbfp5, Igbfp3, Mmp2, Mmp11, or Mmp13.

In some exemplary embodiments, described herein are methods of screening for one or more agents capable of remodeling in a hematological disease comprising:

contacting a cell population with a test modulating agent, wherein the cell population comprises MSC(s), OLC(s), and leukemia cells; and

selecting modulating agents that

a. change the proportion of prerosteoblasts in the cell population;

b. change the relative proportion of OLC-1 to OLC-2 in the cell population;

c. change the relative proportion of hypertrophic chondrocytes to progenitor chondrocytes in the cell population;

d. change the relative proportion of subtype-3 (Cluster 16) fibroblasts to subtype-4 fibroblasts (Cluster 3); or

e. a combination thereof.

In some exemplary embodiments, described herein are methods of detecting a mesenchymal stem/stromal cell (MSC) from a population of stromal cells comprising:

detecting in a sample the expression or activity of a MSC gene expression signature,

wherein detection of the MSC gene expression signature indicates MSCs in the sample, and

wherein the MSC gene expression signature comprises:

a. one or more genes of Table 1;

b. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or

c. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxcl1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1;

and wherein the MCS optionally does not express one or more of Thy1, Ly6a (Sca-1), NG2 (Cspg4) or Nestin (Nes).

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein a pharmaceutically acceptable carrier. When present, the compound can optionally be present in the pharmaceutical formulation as a pharmaceutically acceptable salt. In some embodiments, the pharmaceutical formulation can include, such as an active ingredient, a modulating agent, hematological disease and/or other therapeutic, a stromal cell (e.g. an isolated, enriched, modulated or otherwise engineered stromal cell having a particular gene signature described herein).

Where appropriate, compounds, molecules, compositions, vectors, vector systems, cells, or a combination thereof described in greater detail elsewhere herein can be provided to a subject in need thereof as an ingredient, such as an active ingredient, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered via any suitable method to a subject in need thereof. In some embodiments, the subject in need thereof has or is suspected of having a hematological disease or a symptom thereof. Exemplary hematological diseases are discussed and described elsewhere herein.

### Pharmaceutically Acceptable Carriers and Auxiliary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of auxiliary active agents, including but not limited to, biologic agents or molecules (including but not limited to (e.g. polypeptides, polynucleotides, antibodies and fragments thereof, aptamers, and the like), chemotherapeutics, antineoplasic agents, hormones, antibiotics, antivirals, immunomodulating agents, antinausea, pain modifying compounds (such as opiates), anti-inflammatory agents, antipyretics, antibiotics, and combinations thereof.

### Effective Amounts

In some embodiments, the amount of the primary active agent and/or optional auxiliary active agent can be an effective amount, least effective amount, and/or therapeutically effective amount. The effective amount, least effective amount, and/or therapeutically effective amount can be effective to treat and/or prevent a hematological disease or a symptom thereof, shift one stromal cell type to another, effective to shift on stromal cell state to another, effective to remodel a bone marrow microenvironment from a pathologic or diseased bone marrow microenvironment to a homeostatic or normal bone marrow microenvironment, effective to shift a dysfunctional stromal cell state to a normal or homeostatic cell state, treat a blood cancer or a symptom thereof, or any combination thereof.

The effective amount, least effective amount, and/or therapeutically effective amount of the primary and optional auxiliary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges. In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the auxiliary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, a primary active agent can be present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments, the auxiliary active agent, when optionally present, can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In embodiments where there is an auxiliary active agent contained in the pharmaceutical formulation, the effective amount of the auxiliary active agent will vary depending on the auxiliary active agent.

When optionally present in the pharmaceutical formulation, the auxiliary active agent can be included in the pharmaceutical formulation or can exist as a stand-alone compound or pharmaceutical formulation that can be administered contemporaneously or sequentially with the compound, derivative thereof, or pharmaceutical formulation thereof. In yet other embodiments, the effective amount of the auxiliary active agent can range from about, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total auxiliary active agent pharmaceutical formulation. In additional embodiments, the effective amount of the auxiliary active agent can range from about, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

In embodiments where a primary active agent and/or auxiliary active agent is a population of cells, the effective amount, least effective amount, and or therapeutically effective amount can range from 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to 1×10²⁰ or be any numerical amount or range therein.

### Dosage Forms

In some embodiments, the pharmaceutical formulations described herein can be in a dosage form. The dosage form can be administered to a subject in need thereof. The dosage form can be effective generate specific concentration, such as an effective concentration, at a given site in the subject in need thereof. In some cases, the dosage form contains a greater amount of the active ingredient than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional auxiliary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. a1. (New York, Marcel Dekker, Inc., 1989), “Remington—The science and practice of pharmacy”, 20th ed., Lippincott Williams & Wlkins, Baltimore, Md., 2000, and “Pharmaceutical dosage forms and drug delivery systems”, 6th Edition, Ansel et al., (Media, Pa.: Wlliams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional auxiliary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional auxiliary active ingredient, and/or pharmaceutically acceptable salt thereof where appropriate can be formulated with a paraffinic or water-miscible ointment base. In other embodiments, the primary and/or auxiliary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional auxiliary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g. micronized) compound or salt or solvate thereof, is defined by a Dso₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or auxiliary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, auxiliary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional auxiliary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional auxiliary active ingredient, and/or pharmaceutically acceptable salt thereof where appropriate, such a dosage form can contain a powder base such as lactose, glucose, trehalose, manitol, and/or starch. In some of these embodiments, a primary active agent, auxiliary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, auxiliary active ingredient, and/or pharmaceutically acceptable salt thereof where appropriate per unit dose. In an embodiment, the predetermined amount of primary active agent, auxiliary active ingredient, and/or pharmaceutically acceptable salt thereof where appropriate can be an effective amount, a least effect amount, and/or a therapeutically effective amount. In some embodiments the predetermined amount can be effective to treat and/or prevent a hematological disease or a symptom thereof, shift one stromal cell type to another, effective to shift on stromal cell state to another, effective to remodel a bone marrow microenvironment from a pathologic or diseased bone marrow microenvironment to a homeostatic or normal bone marrow microenvironment, effective to shift a dysfunctional stromal cell state to a normal or homeostatic cell state, treat a blood cancer or a symptom thereof, or any combination thereof.

In other embodiments, the predetermined amount of a primary active agent, auxiliary active agent, and/or pharmaceutically acceptable salt thereof where appropriate, can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

In some embodiments, the primary auxiliary agent, secondary auxiliary agent, or a pharmaceutically acceptable salt thereof where appropriate, can be used as a medicament to treat and/or prevent a hematological disease or a symptom thereof, shift one stromal cell type to another, effective to shift on stromal cell state to another, effective to remodel a bone marrow microenvironment from a pathologic or diseased bone marrow microenvironment to a homeostatic or normal bone marrow microenvironment, effective to shift a dysfunctional stromal cell state to a normal or homeostatic cell state, treat a blood cancer or a symptom thereof, or any combination thereof. In some embodiments, the primary auxiliary agent, secondary auxiliary agent, or a pharmaceutically acceptable salt thereof where appropriate, can be used in the manufacture of a medicament to treat and/or prevent a hematological disease or a symptom thereof, shift one stromal cell type to another, effective to shift on stromal cell state to another, effective to remodel a bone marrow microenvironment from a pathologic or diseased bone marrow microenvironment to a homeostatic or normal bone marrow microenvironment, effective to shift a dysfunctional stromal cell state to a normal or homeostatic cell state, treat a blood cancer or a symptom thereof, or any combination thereof.

### Kits

In another aspect, the invention is directed to kit and kit of parts. The terms “kit of parts” and “kit” as used throughout this specification refer to a product containing components necessary for carrying out the specified methods (e.g., methods for detecting, quantifying or isolating bone marrow stromal cells and/or immune cells as taught herein), packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the recited binding agents(s) as taught herein, such as for example, antibodies, hybridization probes, amplification and/or sequencing primers, optionally provided on arrays or microarrays, the present kits may also include some or all of solvents, buffers (such as for example but without limitation histidine-buffers, citrate-buffers, succinate-buffers, acetate-buffers, phosphate-buffers, formate buffers, benzoate buffers, TRIS (Tris(hydroxymethyl)-aminomethan) buffers or maleate buffers, or mixtures thereof), enzymes (such as for example but without limitation thermostable DNA polymerase), detectable labels, detection reagents, and control formulations (positive and/or negative), useful in the specified methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

In some embodiments the kits can contain directions, provided in any electronic or physical form, that indicate the compounds, formulation(s) and/or molecules, compositions, cells, vector(s) and the like described herein can be effective to and/or used for treat and/or prevent a hematological disease or a symptom thereof, shift one stromal cell type to another, effective to shift on stromal cell state to another, effective to remodel a bone marrow microenvironment from a pathologic or diseased bone marrow microenvironment to a homeostatic or normal bone marrow microenvironment, effective to shift a dysfunctional stromal cell state to a normal or homeostatic cell state, treat a hematological disease or a symptom thereof, or any combination thereof. Exemplary hematological diseases are discussed and described in greater detail elsewhere herein.

### Methods of Using the Bone Marrow Stromal Cell Signatures and Cell Populations

Some observations of the subsets include the following. Notably, osteoblasts segregated into two subsets that appear to arise from distinct lineage trajectories. Only one of these, which emerges from MSCs, expresses HSC regulatory genes and does so at the osteoprogenitor stage of development.

One of the five fibroblasts subsets, termed Fibroblasts-1, expresses Cxcl12. Cxcl12-expressing fibroblasts have been implicated in aggressive solid tumors (Ahirwar et al., 2018; Costa et al., 2018).

Third, ECs were the most abundant cell type in bone marrow stroma, and include a distinctive, immature subset that is enriched for expression of hematopoietic regulators. This is in marked contrast to the large sinusoidal endothelium subset that minimally expresses the HSC niche factors, Cxcl12 and Kit1, despite prior reports that peri-sinusoidal positioning of HSC is common (Acar et al., 2015).

LepR⁺ cells are extremely common across multiple cell types, including osteolineage, endothelial, pericyte and fibroblastic cells. Thus, the use of LepR-driven Cre to alter particular genes could impact their expression across many cell types, and must be interpreted with extreme care, especially where LepR-Cre is expressed throughout development, as commonly done to date. It is demonstrated that LepR expressing cells are quite distinct from Nestin or Cspg4 (Ng2) producing cells. The latter have been previously reported as important HSC niche cells, suggesting that subtle functional distinctions may be discerned between HSCs associated with the distinct niches these cells may represent. Within MSCs, where LepR expression is abundant, Grem1 is disproportionately expressed in particular MSC subsets. Grem1⁺ cells have been described as functionally different than LepR+ cells in their relative ability to make specific mature marrow subsets such as adipocytes (Worthley et al., 2015; Zhou et al., 2014a). As the expression of Grem1 does not track precisely with LepR, these may indeed represent cell populations with graded functional capabilities.

Pericytes were inferred as of distinct lineage from the MSC populations reported to be perivascular (Mendez-Ferrer et al., 2010) and there was discordant expression of Cxcl12 and Kitl among pericyte subpopulations. Pericytes with abundant Cxcl12 also express Cspg4 and Nes, but have little detectable Kitl or LepR. Pericyte charactization and distinction herein provides for assessment of functional distinctions in hematopoietic support, and how this sub-population tracks with periarteriolar, quiescent HSCs (Kunisaki et al., 2013).

Importantly, the presence of acute myeloid leukemia distorted the stromal compartment in select and specific ways. Osteogenic differentiation blockade occurs in MSCs and OLCs, is associated with a hypoxia signature, and accompanied by a cell intrinsic bone remodeling phenotype and disturbed production of hematopoietic regulatory factors that affect normal hematopoiesis (Cxcl12, Kit1, Angpt1 and Spp1). While most studies show that osteoblast numbers are either reduced (Frisch et al., 2012; Krevvata et al., 2014; Kumar et al., 2018) or increased (Schepers et al., 2013), depending on type of leukemia model used, osteoblast classification herein shows a loss of bone maturation phenotype and function. A parenchymal tumor affecting the maturation of tissue stromal cells has not been previously noted and does suggest a distinct type of cross-interaction between emerging cancer cells and their mesenchymal neighbors. It may be that differentiation blockade is not restricted in a cell autonomous manner to cancer cells, but may extend to the broader cell context of a cancerous tissue. Additional cell intrinsic changes associated with AML in specific subclusters impacted HSC support factors expressed by MSCs, osteoprogenitors, endothelial progenitors and arteriolar ECs, with marked reduction in expression of genes important for HSC retention and persistence (Cxcl12, Kitl and Angpt1), hematopoietic maturation (Il7 and Csf1) and cell adhesion (Vcam1).

These findings illuminate the influence of emerging cancer cells on the stromal cells in the tissue they inhabit. They can alter differentiation patterns of those stromal cells, changing the complexity of cell types that are thought to play critical roles in governing tissue homeostasis. Further, the malignant cells in this study are shown to reduce the expression of regulatory signaling molecules known to be essential for normal hematopoietic function. In so doing, the malignant cells create a microenvironment no longer as conducive to normal hematopoietic cell production and, thereby, impairing the parenchymal cells with which they compete. These data provide experimental support for a paradigm in which the establishment of a malignant clone within a tissue shapes the features of the stromal landscape of that tissue. The result compromises stromal cell support of normal parenchymal cells, fundamentally altering the competitive landscape of the tissue to disadvantage normal cells. In this way, cancer cells act not as fully independent and destructive rogues, but rather as self-serving architects of their neighborhood pushing out normal occupants by creating a less supportive environment.

The clusters and subsets provided herein allow a clearer and more consistent picture of the ways in which specific stromal cells contribute to homeostasis and aberrant hematopoiesis, and provide a foundation for developing stromal-targeted therapies in hematologic disease. The unique classifications offer evidence for tumor evolution in which cross-communication between parenchymal and stromal elements influence the emergence of cancer.

### Methods of Diagnosis

Also described herein are methods of diagnosing stromal cell dysfunction and a related disease, such as a hematological disease, or state of disease, in a subject. In some embodiments, the subject can have or be suspected of having or be at risk for developing a stromal cell dysfunction and/or related disease. Such diseases include, but are not limited to, hematological disease or hematological disease. Hematological diseases that can be treated and/or prevented by the inventive compositions and formulations described herein include but are not limited to blood cancers, myelodysplastic syndrome, and polycythemia vera. Blood cancers include, but are not limited to, leukemias, myelomas, and lymphomas. Leukemias include, but are not limited to acute lymphocytic leukemia, acute myeloid leukemia, chronic lymphocytic leukemia, chronic myeloid leukemia, hairy cell leukemia, myelodysplastic syndromes, acute promyelocytic leukemia, and myeloproliferative neoplasm. Myelomas include, but are not limited to, multiple myeloma. Lymphomas include, but are not limited to, Non-Hodgkin lymphoma, Hodgkin lymphoma, Cutaneous B-cell lymphoma, Cutaneous T-cell lymphoma, Waldenstrom macroglobulinemia, and lymphoma of the skin.

In some embodiment, a method of diagnosis can include determining a signature, such as a gene signature, expression profile, module, program or a combination thereof in one or more stromal cells and determining a diagnosis of a disease based on the signature, expression profile, module, program or a combination thereof. Such signatures as well as methods and techniques for detecting a signature, expression profile, module, program, or a combination thereof are described elsewhere herein. In some embodiments, the subject can be diagnosed or be determined to be at risk for developing a stromal cell dysfunction and/or related disease. Such diseases include, but are not limited to, hematological diseases. In some embodiments, the hematological disease is a hematopoietic disease. Hematological diseases and hematological diseases that can be treated and/or prevented by the inventive compositions and formulations described herein include but are not limited to blood cancers myelodysplastic syndrome, polycythemia vera, Blood cancers include, but are not limited to, leukemias, myelomas, and lymphomas. Leukemias include, but are not limited to acute lymphocytic leukemia, acute myeloid leukemia, chronic lymphocytic leukemia, chronic myeloid leukemia, hairy cell leukemia, myelodysplastic syndromes, acute promyelocytic leukemia, and myeloproliferative neoplasm. Myelomas include, but are not limited to, multiple myeloma. Lymphomas include, but are not limited to, Non-Hodgkin lymphoma, Hodgkin lymphoma, Cutaneous B-cell lymphoma, Cutaneous T-cell lymphoma, Waldenstrom macroglobulinemia, and lymphoma of the skin,

In some embodiments, a subject is diagnosed as having or being at risk for a disease when there is a shift in stromal cell state from homeostatic to dysfunctional In some embodiments, a subject is diagnosed as having or being at risk for a disease when there is a shift in stromal cell state from a first stromal cell type selected from Cluster 1-17 as described in the Working Examples herein to a second stromal cell type selected from any one of Cluster 1-17 or a sub-type thereof as described in the Working Examples, where the first and the second cell type are not the same. In some embodiments, a diagnosis is made when the there is a change in the relative proportion of OLC-1 cells to OLC-2 cells is changed as compared to a suitable control. In some embodiments, a diagnosis is made when the number or fraction of OLC-1 cells is increased in the population as compared to a control. In some embodiments, a diagnosis is made when the number or fraction of OLC-2 cells is decreased in the population as compared to a control. In some embodiments, a diagnosis is made when the there is a change in the relative proportion of bone marrow derived endothelial fractions is changed as compared to a suitable control. In some embodiments, a diagnosis is made when the number or fraction of sinusoidal BMECs is decreased in the population as compared to a control. In some embodiments, a diagnosis is made when the number or fraction of arterial BMECs is increased in the population as compared to a control. In some embodiments, a diagnosis is made when the there is a change in the relative proportion of chondrocyte fractions is changed as compared to a suitable control. In some embodiments, a diagnosis is made when the number or fraction of chondrocyte progenitor cell subtype is decreased in the population as compared to a control. In some embodiments, a diagnosis is made when the number or fraction of chondrocyte hypertrophic cell subtype is increased in the population as compared to a control. In some embodiments, a diagnosis is made when the there is a change in the relative proportion of fibroblast subtypes are changed as compared to a suitable control. In some embodiments, a diagnosis is made when the number or fraction of a fibroblast subtype-3 fraction or number is decreased in the population as compared to a control. In some embodiments, a diagnosis is made when the number or fraction of a fibroblast subtype-4 fraction or number is increased in the population as compared to a control. In some embodiments, a diagnosis is made when the there is a change in the relative proportion of MSC fractions are changed as compared to a suitable control. In some embodiments, a diagnosis is made when the number or fraction of a MSC-2 fraction or number is increased in the population as compared to a control. In some embodiments, a diagnosis is made when the number or fraction of a MSC-3 fraction or number is decreased in the population as compared to a control. In some embodiments, a diagnosis is made when the number or fraction of a MSC-4 fraction or number is decreased in the population as compared to a control.

In methods of diagnosing, the method comprises the step of detecting a gene expression profile in one or more cells or tissues associated with stromal cell state and/or stromal cell dysfunction. This is also discussed in greater detail elsewhere herein. The order of steps provided herein is exemplary, certain steps may be carried out simultaneously or in a different order.

In some embodiments, a subject can be diagnosed as having a disease or a specific disease state based on the presence or amount of a cell, such as a stromal cell, that is of a specific cell type or subtype, has a specific cell identity, and/or is in a specific-cell state. In some embodiments, a subject can be diagnosed as having a hematological disease or a specific hematological disease state when a stromal cell is determined, based on one or more of the methods described in greater detail herein, to be in a disease or dysfunctional state. In some aspects, a subject can be diagnosed as having hematological disease, if one or more dysfunctional stromal cells are detected in a biological sample obtained from the subject. Further aspects of these methods will be appreciated by those of ordinary skill in the art in view of the description provided elsewhere herein, as well as, in the Working Examples below.

In some aspects, the method of detecting a hematological disease can include determining a fraction of dysfunctional or diseased stromal cell in a sample from a subject; and diagnosing the hematological disease in the subject when the fraction of diseased or dysfunctional stromal cell in the sample is increased relative to a fraction of homeostatic stromal cells in a non-diseased control. In some aspects, the non-diseased control is an age-matched non-disease control. In some aspects the subject is symptomatic for a hematological c disease. In some aspects, the subject is asymptomatic for a hematological disease.

The methods described here and elsewhere herein can be used to stratify a patient population into previously unknown patient pools, which then can be applied to unexpectedly alter and/or improve patient treatment.

Biomarkers, as discussed above, can be used in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Suitably, an altered quantity or phenotype of the immune cells in the subject compared to a control subject having normal immune status or not having a disease comprising an immune component indicates that the subject has an impaired immune status or has a disease comprising an immune component or would benefit from an immune therapy.

Hence, the methods may rely on comparing the quantity of immune cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value >second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±×SD or ±2×SD or ±3×SD, or ±×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

In certain embodiments, signature genes and biomarkers related to the stromal cell type, subtype, and/or cell state may be identified by comparing single cell expression profiles obtained from healthy or normal cells and diseased cells.

In one particular embodiment, signature genes and biomarkers related to the hematological disease may be identified by comparing single cell expression profiles obtained from uninfected cells and diseased cells.

A gene profile can be a gene signature, or expression profile. In one aspect, the gene expression profile measures upregulation or down regulation of particular genes or pathways and is further defined and described elsewhere herein. In particular instances, the gene expression profile can include one or more genes from genes of a normal stromal cell type, subtype, and/or state gene signature, a modulated stromal cell's cell state, and/or a dysfunction or diseased stromal cell. Stromal cell specific gene expression signatures, space, profiles, and modules (such as dysfunctional stromal cell modules) are described in greater detail elsewhere herein.

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined and described herein elsewhere.

In some embodiments, method of detecting a stromal cell type and/or state or population thereof in a population of cells can include detecting in a sample the expression or activity of a homeostatic and/or dysfunctional stromal cell or cell state expression signature,

wherein detection of the homeostatic and/or dysfunctional stromal cell e cell state xpression signature indicates the presence of homeostatic and/or dysfunctional stromal cell(s) in the sample. In some embodiments, the homeostatic and/or dysfunctional stromal cell state cell expression signature can be composed of one or more biomarkers from any one of Tables 1-8 or a combination thereof, (e.g. any one of Clusters 1-17 or cluster subtypes therein), as otherwise identified in the Working Examples herein.

In some embodiments, the signature used to diagnose a disease, detect dysfunction, and/or detect stromal remodelling can be:

a. an MSC signature, where the MSC signature comprises:
i. one or more genes of Table 1 or gene products thereof;
ii. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, 1134, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or
iii. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Idl, Ifrd1, Trib1, Rrad, Odcd, Actb, Notch2, AlpI, Mmp13, Raph1, Tnfsf11, Cxcl1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxc112, Kit1, Grem1, or Angpt1;

and wherein the MCS optionally does not express one or more of Thy1, Ly6a (Sca-1), NG2 (Cspg4) or Nestin (Nes);

b. an OLC signature, where the OLC signature comprises:
i. one or more genes of Table 2 or gene products thereof;
ii. one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Loxl1, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;
iii. one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmem119, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbpl1, Col3a1, Ptn, Col6a2, Tnn, Npy, Col6a1, Omd, Dcn, Tgfbi, Col6a3, or Acan; or
iv. one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13;

and wherein the OLC optionally expresses Bglap and Spp1;

c. a chondrocyte signature, wherein the chondrocyte gene signature comprises:
i. one or more genes of Table 4 or a gene product thereof;
ii. one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst11, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, Il17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;
iii. one or more of Sox9, Col11a2, Acan, or Col2a1;
iv. one or more of Runx2, Ihh, Mef2c, or Col10a1;
v. one or more of Grem1, Runx2, Sp7, Alp1, or Spp1;
vi. one or more of Ihh, Pth1r, Mef2c, Col10a1, Ibsp, Mmp13, Grem 1; or
vii. one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst1, Epyc, Serpinh1, Gnb211, Fscn1, Pla2g5, Rcn1, Sox9, Bglap, Sp7, Fn1, Ube2s, Hmgb1, Ckap4, Clec11a, Il17b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12;

d. a fibroblast signature, where the fibroblast gene signature comprises:
i. one or more genes of Table 5 or a gene product thereof;
ii. one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;
iii. Fibronectin-1 (Fn1), Fibroblast Specific Protein-1 (S100a4), Col1a1, Col1a2, Lum, Col22a1, or Twist2;
iv. one or more of Sox9, Acan, and Col2a1;
v. Cd34, Ly6a, Pdgfra, Thy1 and Cd44, and not Cdh5; or
vii. one or more of Sox-9, Scleraxis (Scx), Spp1, Cspg4, CD73 (Nt5e), and Cartilage Intermediate Layer Protein (Cilp); or
vii. one or more of S1004a, Dcn, Sema3c, or Cxcl12;

e. a bone marrow derived endotheial cell (BMEC) signature, wherein the BMEC signature comprises:
i. one or more genes of Table 6 or a gene product thereof;
ii. one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x;
iii. one or more of Flt4 (Vegfr-3) or Ly6a (Sca-1);
iv. one or more of Pecam1, Cdh5, Cd34, Tek, Lepr, Cxcl12, or Kitl;
v. one or more of Flt4, Ly6a, Icam1, or Sele;
vi. one or more of Mafb, Cebpb, Xbp1, Nr2f2, Irf8, Ybx1, Ebf1, Sox17, Mxd4, Id1, Meox2, Tshz2, Tcf15, Meox1, Tfpi, I1l6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, lfitm2; or
vii. one or more of Mafb, Cebpb, Xbp1, Nr2f2, Irf8, Ybx1, Ebf1, Sox17, Mxd4, Id1, Meox2, Tshz2, Tcf15, Meox1, Tfpi, I1l6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2

f. a pericyte signature, where the pericyte signature comprises:
i. one or more genes in Table 3 or a gene product thereof;
ii. one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn41r11, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, Cygp;
iii. one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100all; or
iv. one or more of Acta2, Myh11, Mcam, Jag1, or Il6.

The method of diagnosis described herein can include detecting one or more dysfunctional stromal cells from or in a subject.

In some embodiments, a method of detecting dysfunctional stromal cells comprising detection of a gene expression signature of dysfunction selected from the group consisting:

a) a signature comprising or consisting of one or more markers selected from the group consisting of Cxcl12, Adipoq, Kit1, Lepr, Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4;

b) a signature comprising or consisting of one or more markers selected from the group consisting of Bglap, Spp1, Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;

c) a signature comprising or consisting of one or more markers selected from the group consisting of Acta2, Myh11, Mcam, Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, or Cygp;

d) a signature comprising or consisting of one or more markers selected from the group consisting of Sox9, Col11a2, Acan, Col2a1, Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst1, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, Il17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;

e) a signature comprising or consisting of one or more markers selected from the group consisting of S100a4, Fn1, Col1a1, Col1a2, Lum, Col22a1, Twist2, Scx, Barx1, Trps1, Hoxd9, Pitx1, Prrx1, Rora, Prrx2, Meox2, Ebf2, Osr2, Ebf1, Dix3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;

f) a signature comprising or consisting of one or more markers selected from the group consisting of Kdr, Cdh5, Thbd, Emcn, Ly6e, Pecam1 Ly6a, Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x; or

g) a signature comprising or consisting of two or more markers each independently selected from any one of the groups as defined in any one of a) to f).

Described herein are methods of determining a dysfunctional phenotype in a stromal cell. In some embodiments, the method can include determining in said cell the expression of the signature of dysfunction as defined above, whereby expression of the signature indicates that the stromal cell has a dysfunctional immune phenotype.

Described elsewhere herein are biomarkers (e.g., phenotype specific or cell type) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described elsewhere herein.

In some embodiments, the method of diagnosing a disease, including a a hematological disease, and/or stromal cell landscape remodeling can include a. determining a fraction of: i. OLC-1 cells, ii. OLC-2 cells, iii. bone marrow derived endothelial cells (BMECs); iv. chondrocytes; v. fibroblasts; and b. diagnosing the neurodegenerative disease in the subject when i. the relative proportion of OLC-1 cells to OLC-2 cells is changed as compared to a suitable control; ii. the fraction of OLC-1 cells is increased as compared to a suitable control; iii. the fraction of OLC-2 cells is decreased as compared to a suitable control; iv. the relative proportion of bone marrow derived endothelial fractions is changed as compared to a suitable control; v. a fraction of sinusoidal BMECs is decreased as compared to a suitable control; vi. a fraction of arterial BMECs is increased as compared to a suitable control; vii. the relative proportion of chondrocyte fractions is changed as compared to a suitable control; viii. a chondrocyte hypertorphic cell subtype is increased as compared to a suitable control; ix. a chondrocyte progenitor cell subtype is decreased as compared to a suitable control; x. a fibroblast subtype is changed as compared to a suitable control; xi. a fibroblast subtype-3 is decreased; as compared to a suitable control xii. a fibroblast subtype-4 is increased as compared to a suitable control; xiii. the relative proportion of MSC fractions is changed as compared to a suitable control; ixx. a MSC-2 fraction is increased as compared to a suitable control; xx. a MSC-3 fraction is decreased as compared to a suitable control; xxi. a MSC-4 fraction is decreased as compared to a suitable control; or xxii. a combination thereof.

Diseases, such as hematological diseases, that can be diagnosed and/or detected are described elsewhere herein.

### Methods of Treatment

Also described herein are methods of treating a subject in need thereof by administering one or more of the polynucleotides, polypeptides, vectors, cells, modulating agent, therapeutic agent as described elsewhere herein, a combination thereof, or a pharmaceutical agent thereof to the subject in need thereof. As used herein, “administering” refers to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. As used herein, “agent” refers to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

The subject in need thereof can have or be suspected of having a hematological disease, be suspected of having a hematological disease, or be at risk for developing a hematological disease, stromal cell dysfunction, and/or related disease. Exemplary hematological diseases are discussed in greater detail elsewhere herein. Hematological diseases that can be treated and/or prevented by the inventive compositions and formulations described herein include but are not limited to blood cancers myelodysplastic syndrome, polycythemia vera. Blood cancers include, but are not limited to, leukemias, myelomas, and lymphomas. Leukemias include, but are not limited to acute lymphocytic leukemia, acute myeloid leukemia, chronic lymphocytic leukemia, chronic myeloid leukemia, hairy cell leukemia, myelodysplastic syndromes, acute promyelocytic leukemia, and myeloproliferative neoplasm. Myelomas include, but are not limited to, multiple myeloma. Lymphomas include, but are not limited to, Non-Hodgkin lymphoma, Hodgkin lymphoma, Cutaneous B-cell lymphoma, Cutaneous T-cell lymphoma, Waldenstrom macroglobulinemia, and lymphoma of the skin.

In some embodiments, it can be determined if a patient would benefit from a treatment that includes reducing dysfunction of one or more stromal cell(s) or stromal cell population(s). In some embodiments, the method of determining if a patient would benefit from a treatment that includes reducing dysfunction of one or more stromal cell(s) or stromal cell population(s) can include determining, in cells from said patient the expression of the signature of dysfunction as defined above, whereby expression of the signature indicates the patient will benefit from the therapy; or for determining whether or not a patient would benefit from a therapy aimed at increasing dysfunction of stromal cells or a subset thereof, or a therapy aimed at downregulating of a stromal response, the method comprising determining, in stromal cells from said patient the expression of the signature of dysfunction as defined above, whereby expression of the signature indicates the patient will likely not benefit from the therapy.

In some embodiments, the efficacy of a treatment of a patient with a therapy, such as a treatment described herein, can be determined. In some embodiment, the method of determining the efficacy of a treatment described herein can include determining in stromal cells from said patient the expression of the signature of dysfunction as defined above before and after said treatment and determining the efficacy of said therapy based thereon.

In some exemplary embodiments, the method of treating can include remodeling a stromal cell landscape comprising administering a modulating agent to a subject or a cell population that induces a shift in the stromal cell landscape from a disease-associated stromal cell landscape to a homeostatic stromal cell landscape.

In some exemplary embodiments, the shift in stromal cells from a disease-associated stromal cell landscape to a homeostatic stromal cell landscape comprises a change in the proportion of preosteoblasts. In some exemplary embodiments, the change in the proportion of preosteoblasts comprises a change in the relative proportion of OLC-1 cells to OLC-2 cells. In some exemplary embodiments, the change in the relative proportion of OLC-1 cells to OLC-2 cells comprises a decrease in OLC-1 cells and an increase in OLC-2 cells.

In some exemplary embodiments, the shift in stromal cells from a disease-associated stromal cell landscape to a homeostatic stromal cell landscape comprises a change in the relative proportion of bone marrow derived endothelial cell subtypes. In some exemplary embodiments, the change in the relative proportion of bone marrow derived endothelial cell subtypes comprises an increase in sinusoidal bone marrow derived endothelial cells and a decrease in arterial bone marrow derived endothelial cells.

In some exemplary embodiments, the shift in stromal cells from a disease-associated stromal cell landscape to a homeostatic stromal cell landscape comprises a change in the relative proportion of chondrocyte subtypes. In some exemplary embodiments, the change in the relative proportion of chondrocyte subtypes comprises a decrease in chondrocyte hypertrophic cell subtype and an increase in chondrocyte progenitor cell subtype.

In some exemplary embodiments, the shift in stromal cells from a disease-associated stromal cell landscape to a homeostatic stromal cell landscape comprises a change in the relative proportion of fibroblast subtypes. In some exemplary embodiments, the change in the relative proportion of fibroblast subtypes comprises an increase in fibroblast subtype-3 and a decrease in fibroblast subtype-4.

In some exemplary embodiments, the shift in stromal cells from a disease-associated stromal cell landscape to a homeostatic stromal cell landscape comprises a change in the relative proportion in mesenchymal stem/stromal cell (MSC) subtypes. In some exemplary embodiments, the change in the relative proportion in mesenchymal stem/stromal cell (MSC) sub-types comprises a decrease in MSC-2 subtype and an increase in MSC-3 and MSC-4 subtypes.

In some exemplary embodiments, the shift in the stromal cell landscape comprises a change in the distance in gene expression space between OLC-1, OLC-2, bone marrow derived endothelial cell subtypes, chondrocyte subtypes, fibroblast subtypes, mesenchymal stem/stromal cell (MSC) subtypes, or a combination thereof. In some exemplary embodiments, the distance is measured by a Euclidean distance, Pearson coefficient, Spearman coefficient, or a combination thereof. In some exemplary embodiments, the gene expression space comprises 10 or more genes, 20 or more genes, 30 or more genes, 40 or more genes, 50 or more genes, 100 or more genes, 500 or more genes, or 1000 or more genes. In some exemplary embodiments, remodeling the stromal cell landscape comprises increasing or decreasing the expression of one or more genes, gene programs, gene expression cassettes, gene expression signatures, or a combination thereof. In some exemplary embodiments, the change in the gene expression space is characterized by a change in the expression of one or more genes as in any of Tables 1-8 or an expression signature derived therefrom. In some exemplary embodiments, identifying differences in stromal cell states in the shift in the stromal cell landscape comprises comparing a gene expression distribution of a stromal cell type or subtype in the diseased stromal cell landscape with a gene expression distribution of the stromal cell type or subtype in the homeostatic stromal cell landscape as determined by single cell RNA-sequencing (scRNA-seq).

In some exemplary embodiments, the shift in the stromal cell landscape from a disease-associated stromal cell landscape to a homeostatic stromal cell landscape increases committed MSCs and decreases osteoprogenitor cells.

In some exemplary embodiments, the subject suffers from a hematological disease. In some embodiments, the hematological disease is a hmatopoeitic disease. In some exemplary embodiments, the hematological disease is a blood cancer. In some embodiments, the blood cancer is leukemia. In some embodiments, the blood cancer is acute lymphocytic leukemia, acute myeloid leukemia, chronic lymphocytic leukemia, chronic myeloid leukemia, hairy cell leukemia, myelodysplastic syndromes, acute promyelocytic leukemia, or myeloproliferative neoplasm.

In some exemplary embodiments, described herein are methods of treating a hematological disease comprising: administering to a subject in need thereof the isolated or engineered cell or cell population as described in greater detail herein.

In some exemplary embodiments, the method of treatment can include screening for a modulating agent capable of modulating a stromal cell state and/or type in the subject to be treated or a cell thereof and administering one or more modulating agents identified by the screening to the subject or a cell there of. Methods of screeing of modulating agents capable of modulating a stromal cell states and/or types are described in greater detail elsewhere herein.

### Diseases

The signatures, biomarkers, cells, compositions, compounds, formulations, and combinations thereof described herein can be used to determine, in some embodiments, presence, state, and/or risk of stromal cell dysfunction and/or related disease. Such diseases include, but are not limited to, a hematological disease. Hematological diseases include, but are not limted to, hematological cancers. Solid tumors can also be treated. Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, myelodysplasia, and blastic plasmacytoid dendritic cell neoplasm.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

The stromal cells and subsets of the invention are useful to study and modify the influence of cancers of all types on metabolic processes. The stromal cells and subsets can be co-cultured with cells of hematological or other cancers. Alternatively, cancer cells can be grafted in animal models and the stromal cells and subsets and their interactions with cancer cells determined. Depending on model, the stromal cells may be autologous, allogeneic or xenogeneic with respect to the cancer cells.

### Stromal Cell Modulation and Cell-Based Treatments

Methods of modulating and engineering stromal cells are described elsewhere herein. In some embodiments, the stromal cells can be modulated in vivo or in situ. In these embodiments, a modulating agent can be administered to a subject in need thereof. The modulating agent can act to modulate the cell, e.g. a stromal cell, from one cell state to another. For example, the modulating agent can act to shift the cells state from a dysfunctional state to a homeostatic or normal cell state. In some embodiments, the modulating agent can act to shift the cell type from a first cell type to a second cell type, where each cell type is defined by a signature. In some embodiments, a cell that is modulated in vivo or in situ can be harvested, optionally cultured and/or expanded ex vivo, and administered to a subject in an autologous or allogeneic manner. This is also further discussed elsewhere herein.

In some embodiments, cells (e.g. stromal cells) are obtained from a source, such as a subject in need thereof. The cells are then optionally cultured and expanded. In some embodiments the cells can be analyzed to determine cell state and optionally sorted. Cells having a homeostatic state can be administered to a subject in an autologous or allogenic manner. Cells having an active state can be administered to a subject in an autologous or allogenic manner. In some embodiments, cells having a homeostatic state can be modulated ex vivo according to methods described elsewhere herein by a modulating agent to change the cell state from one to another. In some aspects, a homeostatic cell can be modulated into an activated cell. In some aspects a dysfunctional stromal cell can be modulated into a homeostatic or normal stromal cell. The modulated cells can then be administered to a subject in need thereof in an autologous or allogenic manner.

The isolated cells and/or engineered cells (e.g. isolated and/or engineered stromal cells(s)) or other cells described herein can be administered to a subject in need thereof. In some embodiments, the isolated cells and/or engineered cells (e.g. isolated and/or engineered stromal cell(s)) or other cells described herein can be combined with one or more additional components to produce formulations, such as pharmaceutical formulations that can be administered to a subject in need thereof. In some aspects, the subject in need thereof can have or be suspected of having a disease as described elsewhere herein. In some embodiments, the cell(s) of the instant invention can be combined with one or more pharmaceutically acceptable carriers or diluents to produce a pharmaceutical composition (which may be for human or animal use). Suitable carriers and diluents include, but are not limited to, isotonic saline solutions, for example phosphate-buffered saline. The composition of the invention may be administered by direct injection. The composition may be formulated for parenteral, intramuscular, intravenous, subcutaneous, intraocular, oral, transdermal administration, or injection into the spinal fluid.

Formulations including isolated and/or engineered cells may be delivered by injection, implantation, or any other suitable method. Cells may be delivered in suspension or embedded in a support matrix such as natural and/or synthetic biodegradable matrices. Natural matrices include, but are not limited to, collagen matrices. Synthetic biodegradable matrices include, but are not limited to, polyanhydrides and polylactic acid. These matrices may provide support for fragile cells in vivo. Delivery may also be by controlled delivery, i.e., delivered over a period of time which may be from several minutes to several hours or days. Delivery may be systemic (for example by intravenous injection) or directed to a particular site of interest. Cells may be introduced in vivo using liposomal transfer.

Cells may be administered in doses of from 1×10⁵ to 1×10′ cells per kg. For example, a 70 kg patient may be administered 1.4×10⁶ cells for reconstitution of tissues. The dosages may be any combination of the target cells listed in this application. Other dosages and forms are discussed elsewhere herein. Additional formulations such as pharmaceutical formulations that can include one or more isolated and/or engineered cells of the instant invention are described elsewhere herein.

### Companion and Personalized Therapies.

Described herein are methods of treating a hematological disease in a subject in need thereof, that can include detecting a hematological disease in the subject by detecting a stromal cell state as described elsewhere herein; and administering an effective amount of a hematological disease treatment to the subject where the fraction of dysfunctional or diseased cells in the sample is increased relative to a non-diseased control. Also described herein are methods of treating a hematological disease in a subject, by administering a hematological disease treatment to the subject in need thereof, wherein the subject is asymptomatic, but where the fraction of dysfunctional or diseasedcells in the sample is increased relative to a non-diseased control.

In some aspects, a method of detecting a cell state, such as the cell state of a stromal cell, and/or diagnosing a hematological disease can be performed as described elsewhere herein, can be performed followed by treating a subject in need thereof using an appropriate treatment method(s). Appropriate treatment methods are described elsewhere herein and include, but are not limited to cell-based treatments (including administering isolated, enriched, modulated, and/or engineered stromal cell(s) described herein to a subject in need thereof, administering a hematological disease therapeutic, and/or a pharmaceutical formulation described herein to a subject in need thereof, modulating a stromal cell, and combinations thereof.

In some embodiments, an agent capable of treating a hematological disease or a symptom thereof to the subject in need thereof. In some embodiments, the agent capable of treating a hematological disease or disorder is a chemotherapeutic agent. In some embodiments, the agent can be cladribine, brentuximab vedotin, polatuzumab vedotin-piiq, fludarabine, fludarabine phosphate, mitoxantorone, etoposide, 6-thioguanine, hydroxyurea, methotrexate, 6-mercaptopurine, azacytidine, decitabine, daunorubicin, cyclophosphamide, daurismo, dexamethasome, cytarabine, arsenic trioxide, nelarabine, asparginase Erwinia chrysanthemi, calaspargase Pegol-mknl, inotuzumab ozogamicin, blinatumomab, clofarbine, dasatinib, dexamethasone, doxorubicin, imatinib mesylate, ponatinib, tisagenlecleucel, vincristine sulfate liposome, vincristine sulfate, mercaptopurine, methotrexate, pegaspargase, prednisone, hyper-CVAD, glasdegib maleate, enasidenib mesylate, gemtuzumab ozogamicin, gilteritinib fumarate, idarubicin, ivosidenib midostaurin, mitoxantrone, thioguanine, venetoclax, gilteritinib fumarate, tagraxofusp-erzs, acalabrutinib, alemtuzumab, ofatumumab, bendamustine HCl, chlorambucil, duvelisib, ibrutinib, idelalisib, mechlorethamine HCl, obinutuzumab, rituximab, hyaluronidase, idelalisib, bosutinib, hydroxyurea, busulfan, nilotinib, omacetaxine mepesuccinate, interferon alpha-2b, moxetumomab pasudotox-tdfk, bortezomib, romidepsin, belinostat, an immune checkpoint inhibitor (e.g. PD-1 inhibitors (e.g. pembrolizumab, nivolumab, and cemiplimab), PD-L1 inhibitors (e.g. atezolizumab, avelumab, and durvalumab), CTLA-4 targeting agents (e.g. ipilimumab), an immunomodulating agent (e.g. thalidomide and lenalidomide), a chimeric antigen receptor (CAR)-T cell therapy (e.g. axicabtagene ciloleucel and tisagenlecleucel), carboplatin, oxaliplatin, pentostatin, gemcitabine, pralatrexate, bleomycin, campath, acalabrutinib, zanubrutinib, idelalisib, copanlisib, duvelisib, and combinations thereof.

In some embodiments, after detection of one or more diseased or dysfunctional stromal signatures described herein is detected in a subject or a sample therefrom, a hematological disease treatment can be administered. In some embodiments, the treatment is an agent capable of treating a hematological disease or a symptom thereof. In some embodimetns the treatment is a chemotherapeutic agent. In some embodiments, the agent is selected from the group of cladribine, brentuximab vedotin, polatuzumab vedotin-piiq, fludarabine, fludarabine phosphate, mitoxantorone, etoposide, 6-thioguanine, hydroxyurea, methotrexate, 6-mercaptopurine, azacytidine, decitabine, daunorubicin, cyclophosphamide, daurismo, dexamethasome, cytarabine, arsenic trioxide, nelarabine, asparginase Erwinia chrysanthemi, calaspargase Pegol-mknl, inotuzumab ozogamicin, blinatumomab, clofarbine, dasatinib, dexamethasone, doxorubicin, imatinib mesylate, ponatinib, tisagenlecleucel, vincristine sulfate liposome, vincristine sulfate, mercaptopurine, methotrexate, pegaspargase, prednisone, hyper-CVAD, glasdegib maleate, enasidenib mesylate, gemtuzumab ozogamicin, gilteritinib fumarate, idarubicin, ivosidenib midostaurin, mitoxantrone, thioguanine, venetoclax, gilteritinib fumarate, tagraxofusp-erzs, acalabrutinib, alemtuzumab, ofatumumab, bendamustine HCl, chlorambucil, duvelisib, ibrutinib, idelalisib, mechlorethamine HCl, obinutuzumab, rituximab, hyaluronidase, idelalisib, bosutinib, hydroxyurea, busulfan, nilotinib, omacetaxine mepesuccinate, interferon alpha-2b, moxetumomab pasudotox-tdfk, bortezomib, romidepsin, belinostat, an immune checkpoint inhibitor (e.g. PD-1 inhibitors (e.g. pembrolizumab, nivolumab, and cemiplimab), PD-L1 inhibitors (e.g. atezolizumab, avelumab, and durvalumab), CTLA-4 targeting agents (e.g. ipilimumab), an immunomodulating agent (e.g. thalidomide and lenalidomide), a chimeric antigen receptor (CAR)-T cell therapy (e.g. axicabtagene ciloleucel and tisagenlecleucel), carboplatin, oxaliplatin, pentostatin, gemcitabine, pralatrexate, bleomycin, campath, acalabrutinib, zanubrutinib, idelalisib, copanlisib, duvelisib, and combinations thereof. In some embodiments, the treatment can be or include a stromal cell or cell population described herein.

In some embodiments, after detection of one or more diseased or dysfunctional stromal signatures described herein is detected in a subject or a sample therefrom, a dosage of a hematological disease treatment that is already being administered to the subject can be modulated. In some embodiments, the dosage can be increased in amount and/or frequency of administration. In some embodiments, the dosage can be increased 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 fold or more.

In some embodiments, a dosage of a hematological disease treatment that is already being administered to the subject can be modulated. In some embodiments, the dosage can be decreased in amount and/or frequency of administration. In some embodiments, the dosage can be decreased 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 fold or more.

Further embodiments are illustrated in the following Examples which are given for illustrative purposes only and are not intended to limit the scope of the invention.

### EXAMPLES

### Example 1—Methods for Examples 2-11

Mice.

The MLL-AF9 knock-in mice (Corral et al., 1996) and CD45.1 (STEM) mice (Morrissy et al., 2016) were described previously. Littermates were used as controls for all experiments involving MLL-AF9 knockin and CD45.1 (STEM) mice. Male C57BL/6 mice (CD45.2, Jackson Laboratory) at age 6-8 weeks were employed as transplant recipients and for steady-state scRNA-seq experiments. All animal experiments were performed in accordance with national and institutional guidelines. Mice were housed in the Massachusetts General Hospital (MGH) Animal Research Facility on a 12 hour light/dark cycle with stable temperature (22° C.) and humidity (60%). All procedures were approved by MGH Internal Animal Care and Use Committee.

Bone Marrow Transplantation and Generation of Leukemic Mice.

To generate leukemic mice, we first crossed the MLL-AF9 knock-in mice (Corral et al., 1996) with the CD45.1 (STEM) mice (Mercier et al., 2016) to generate donor chimeric CD45.1.2 mice. Mice positive for the MLL-AF9 fusion transgene were used as donors, and littermates negative for the MLL-AF9 fusion transgene were used as controls. Mice were sacrificed via CO₂ asphyxia; tibiae and femurs were harvested and excess soft tissue was eliminated. Bones were crushed and washed in PBS and passed through a 70 μm filter into a collection tube and 1×10⁶ whole bone-marrow cells were transplanted by retro-orbital injection into 6-8 weeks old male CD45.2 C57BL/6 recipient mice. One day prior to transplantation, mice were subjected to whole body irradiation (2×6Gy) with a 6-hour interval from a ¹³⁷Cs source. Monthly retro-orbital bleeding was performed on isoflurane anesthetized mice and blood was withdrawn using heparinized capillaries and collected into EDTA containing tubes to prevent coagulation. Complete blood counts were done using the Element Ht5 Auto Hematology analyzer. Subsequently, RBCs were lysed as described previously and cells were stained in PBS, 2% FBS using the following antibodies: CD45.2-APCCy7 (Biolegend, Ref #109824, clone 102), CD45.1-FITC (Biolegend, Ref #110706, clone A20), CDllb-Alexa Fluor 700 (Ref #101222, clone M1/70), GR1-Brilliant violet 570 (Biolegend, Ref #108431, clone RB6-8C5), B220-eFluor450 (eBioscience, Ref #48-0452-82, clone RA3-6B2), and CD3e-APC (eBioscience, Ref #17-0031-83, clone 145-2c11), in addition to 7-Aminoactinomycin D (7AAD; ThermoFisher Scientific, Ref # A1310) for viability to monitor donor chimerism within the different lineages and the appearance of leukemic blasts characterized by a distinct scatter and lower GR1 and CDllb expression within the myeloid compartment. Leukemic mice were determined by the combination of disease symptoms, white blood cell counts and appearance of leukemic blasts.

Isolation of Bone Marrow Stroma Cells.

To obtain bone marrow niche cells for scRNA-seq, mice were sacrificed via CO₂ asphyxia. Bones (femur and tibia) were harvested and placed in Media 199 (ThermoFisher Scientific, Ref #12350039) supplemented with 2% Fetal Bovine Serum (FBS, ThermoFisher Scientific, Ref #10082147). Muscle and tendon tissue was removed and bone marrow was flushed. Niche cells from the bone marrow fraction were isolated by digestion with 1 mg/mL STEMxymel (Worthington, Ref # LS004106) and 1 mg/mL Dispase 1 (ThermoFisher Scientific, Ref #17105041), in Media 199 supplemented with 2% FBS for 25 min at 37° C. Niche cells from the bone fraction were isolated by gently crushing and cutting bones into small fragments and digested in the same digestion mix as the bone marrow for 25 min, at 37° C. with agitation (120 rpm). After digestions, both fractions were filtered through a 70m filter into a collection tube (Fisher Scientific, Ref #08-771-2), pooled into one sample, and erythrocytes lysed in ACK-lysis buffer (ThermoFisher Scientific, Ref # C1430) for 5 minutes on ice. Cells were then stained in Media 199 supplemented with 2% FBS for FACS cell sorting.

FACS Enrichment of Bone Marrow Niche Cells.

For flow cytometry and FACS, cells were resuspended in Media 199 supplemented with 2% FBS and stained for Ter119-APC (eBioscience, Ref #17-5921-82, clone TER-119), CD71-PECy7 (Biolegend, Ref #113812, clone RI7217), CD45-PE (eBioscience, Ref #12-0451-82, clone 30-F11), CD3-PE (Biolegend, Ref #100206, clone 17A2), B220-PE (Biolegend, Ref #103208, clone RA3-6B2), CD19-PE (Biolegend, Ref #115508, clone 6D5), Gr-1-PE (Biolegend, Ref #108408, clone RB6-8C5), and Cd11b-PE (Biolegend, Ref #101208, clone M1/70) for 30 minutes on ice. Dead cells and debris were excluded by FSC, SSC, DAPI (4′,6-diamino-2-phenylindole, Life Technologies, Ref # D3571) and Calcein AM staining profiles and Calcein AM (Life Technologies, Ref # C1430). FACS and cytometry was performed on a BD FACSAria II sorter, and sorted bone marrow niche cells were collected in Media 199 supplemented with 2% FBS and 0.4% UltraPure BSA (Life Technologies, Ref # AM2616). Bone marrow stroma was enriched by sorting of live cells (7-AAD−/Calcein+) negative for erythroid (CD71/Ter119) and immune lineage markers (CD45/CD3/B220/CD19/Gr-1/CD11b).

Single Cell RNA-Seq.

Single cells were encapsulated into emulsion droplets using Chromium Controller (10× Genomics). scRNA-seq libraries were constructed using Chromium Single Cell 3′ v2 Reagent Kit according to the manufacturer's protocol. Briefly, post sorting sample volume was decreased and cells were examined under a microscope and counted with a hemocytometer. Cells were then loaded in each channel with a target output of ˜4,000 cells. Reverse transcription and library preparation were performed on C1000 Touch Thermal cycler with 96-Deep Well Reaction Module (Bio-Rad). Amplified cDNA and final libraries were evaluated on a Agilent BioAnalyzer using a High Sensitivity DNA Kit (Agilent Technologies). Individual libraries were diluted to 4 nM and pooled for sequencing. Pools were sequenced with 75 cycle run kits (26 bp Readl, 8 bp Indexl and 55 bp Read2) on the NextSeq 500 Sequencing System (Illumina) to ˜70-80% saturation level.

Pre-Processing of scRNA-Seq Data.

ScRNA-Seq data were demultiplexed, aligned to the mouse genome, version mml0, and UMI-collapsed with the Cellranger toolkit (version 2.0.1, 10× Genomics). Cells with fewer than 500 detected genes (where each gene had to have at least one UMI aligned) were excluded. Gene expression was represented as the fraction of its UMI count with respect to total UMI in the cell and then multiplied by 10,000, denoted by TP10K—transcripts per 10K transcripts.

Dimensionality Reduction.

Dimensionality reduction was performed using gene expression data for a subset of variable genes. The variable genes were selected based on dispersion of binned variance to mean expression ratios using FindVariableGenes function of Seurat package (Satija et al., 2015) followed by filtering of cell-cycle, ribosomal protein, and mitochondrial genes. Next, we performed principal component analysis (PCA) and reduced the data to the top 50 PCA components (number of components was chosen based on standard deviations of the principal components—in a plateau region of an “elbow plot”).

Clustering and Sub-Clustering.

Graph-based clustering of the PCA reduced data with the Louvain Method (Blondel et al., 2008) was used after computing a shared nearest neighbor graph (Satija et al., 2015). We visualized the clusters on a 2D map produced with t-distributed stochastic neighbor embedding (t-SNE) (Maaten and Hinton, 2008). For sub-clustering, we applied the same procedure of finding variable genes, dimensionality reduction, and clustering to the restricted set of data (usually restricted to one initial cluster).

Differential Expression and Cluster-Specific Gene Signatures.

For each cluster, the Wilcoxon Rank-Sum Test was used to find genes that had significantly different RNA-seq TP10K expression from the remaining clusters (after multiple hypothesis testing correction). As a support measure for ranking differentially expressed genes, the area under receiver operating characteristic (ROC) curve was also used.

Filtering Out Hematopoietic Clusters and Suspected Doublets.

Based on cluster annotations with characteristic genes, hematopoietic clusters were removed from further analysis. It is further expected that a small fraction of data should consist of cell doublets (and to an even lesser extent of higher order multiplets) due to co-encapsulation into droplets and/or as occasional pairs of cells that were not dissociated in sample preparation. Therefore, when found, small clusters of cells expressing both hematopoietic and stromal markers were removed from further analysis. A small number of additional clusters was marked by genes differentially expressed in at least two larger stromal clusters and were annotated as doublets if their average number of expressed genes was higher than the averages for corresponding suspected singlet cluster sources and/or they were not characterized by specific differentially expressed genes. All marked doublets were removed from further analysis.

Estimation of Proliferation Status.

To score cells for their relative proliferation status, a set of characteristic genes involved in cell-cycle (Kowalczyk et al., 2015) was used. For each cell the average expression (TP10K) of cell-cycle genes was computed as a proxy for proliferation status.

Diffusion Maps Computation and Visualization.

Non-linear dimensionality reduction of scRNA-seq data was performed by restricting a sparse diffusion matrix of expression data to the eigenspace spanned by eigenvectors corresponding to the top diffusion matrix eigenvalues. The destiny package (Angerer et al., 2016) was employed, using the local estimation of Gaussian kernel width, and the number of nearest neighbors for diffusion matrix approximation was set to the smaller value between the square root of the number of all single cells in the data and 100. The diffusion matrix was built on sets of variable genes computed with the same procedure as the one used for clustering and sub-clustering of the data, i.e. variable genes were re-computed for each diffusion map.

Diffusion maps were visualized following the approach described in (Schiebinger et al., 2017). Specifically, a nearest-neighbor graph was built in the projected space using an implementation of k-NN algorithm from the package FNN (Beygelzimer et al., 2015), and then a force-directed layout was computed using ForceAtlas2 (Jacomy et al., 2014) from the Gephi package (Bastian et al., 2009).

Connectivity of Cell Clusters.

Connectivity of single-cell clusters was quantified using the partition based graph abstraction method PAGA (Wolf et al., 2017a), a part of the single-cell analysis package Scanpy (Wolf et al., 2018). The computations were carried out on the same subset of variable genes as for clustering, using default parameters.

Cell Identity Assignment in the Leukemia scRNA-Seq Dataset.

For scRNA-seq data from control and leukeic mice, hematopoietic cells were removed from the data as described above, and cell types/identities assigned to each cell using differential expression signatures derived from steady state data. Specifically, we collected up to 50 top, most differentially expressed genes in each of the 20 original clusters from the homeostasis data set as signatures. For each candidate cell, the signature scores for each of the 20 signatures was computed. Each signature score was computed against a background gene set of randomly selected genes. A cell was assigned to the cell-type/cluster with the best signature score. When assigning the sub-cluster identity, we further scored against signature genes of sub-clusters that consisted of up to 20 most differentially expressed sub-cluster genes.

Changes in Cluster Sizes in Leukemia Vs. Control.

For each cluster, the Wald test was used to quantify the association of condition (control, leukemia) with binomial models. Specifically, for each sample, we collected numbers of cells belonging to a given cluster and the number of cells outside of the cluster. Then we fit a generalized linear model with binomial parameters to the combined data with and without a parameter indicating condition (CTRL, Leukemia). An R implementation of Wald was used test to assess the statistical significance of the difference between the two models. Finally, we corrected the Wald test p-values from all clusters for multiple-hypothesis testing (Benjamini and Hochberg, 1995).

Differential Expression in Leukemia Vs. Control.

Some contamination of single cell data was observed likely due to ambient RNA. Therefore, prior to computing differentially expressed genes between leukemia and control, we filtered out those genes that were differentially expressed in hematopoietic contingent of the data when compared with the stromal contingent. The filtered genes were identified separately in leukemia and control conditions since their respective hematopoietic contingents are known to be different. Specifically, this was achieved by computing differentially expressed genes using Bonferroni corrected Wilcoxon Sum-Rank Test as implemented in FindAllMarkers function (default parameters) of the Seurat package and excluding genes from hematopoietic clusters with adjusted p-values <0.05.

Then, separately for each cluster, genes that were differentially expressed between leukemia and control were computed in two ways. First, Bonferroni corrected Wilcoxon Rank-Sum Test (using FindMarkers of Seurat package, logfc.threshold=ln(1.2)) was used to discover differentially expressed genes between conditions. Second, for each cluster, average TP10K expression of cells was computed in every replicate. Those values were used in a t-test to assess differences between leukemia and control conditions. This approach mimics bulk RNA-seq measurements. The second approach, although less powerful for discovery of differentially regulated genes, helped us to identify genes that tended to be coherently regulated in samples.

Gene set enrichment analysis. To identify pathways and cellular states having induced or repressed expression in each cell cluster, Gene Set Enrichment Analysis (GSEA) with MSigDB (Subramanian et al., 2005) gene sets was used. Specifically, the pre-ranked analysis mode was used, with gene transcripts ranked according to differential expression analysis results (Wilcoxon Runk-Sum Test) of comparing leukemic and control conditions in each cluster. The most significantly over-expressed genes were placed at the top of the ranked list, while the most under-expressed were at the bottom before running the test.

### Example 2—an scRNA-Seq Census of Bone Marrow Stroma Identifies MSCs, OLCs, Chondrocytes, Fibroblasts, ECs and Pericytes, and their Differentiation Relations

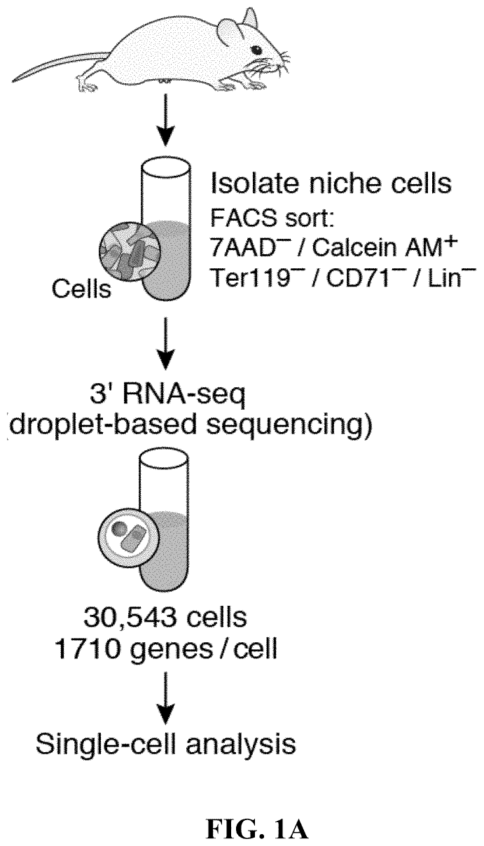

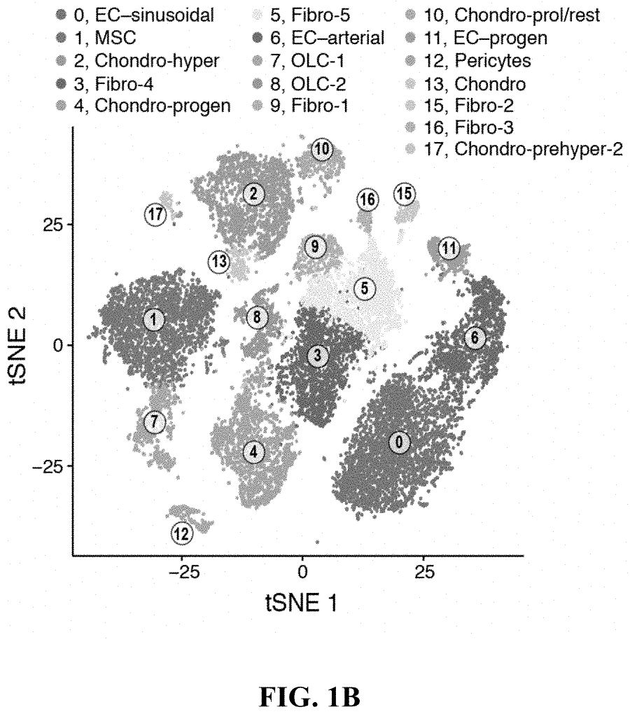

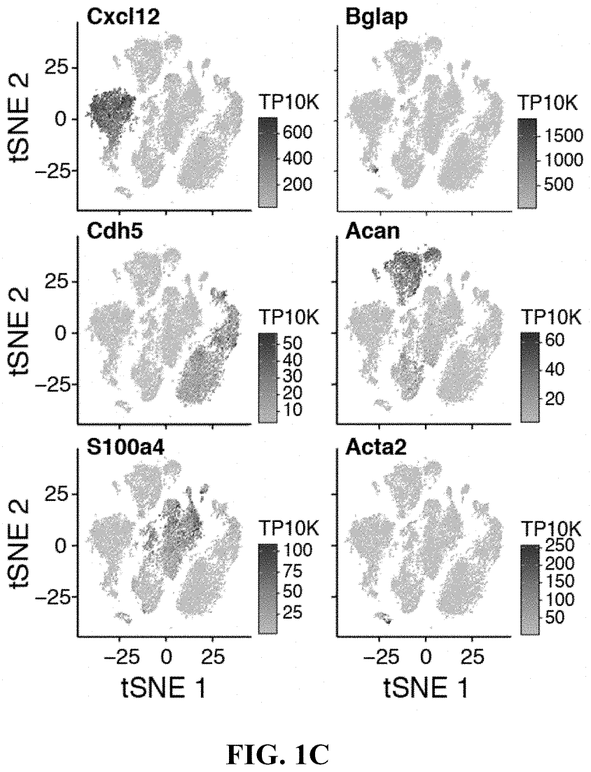

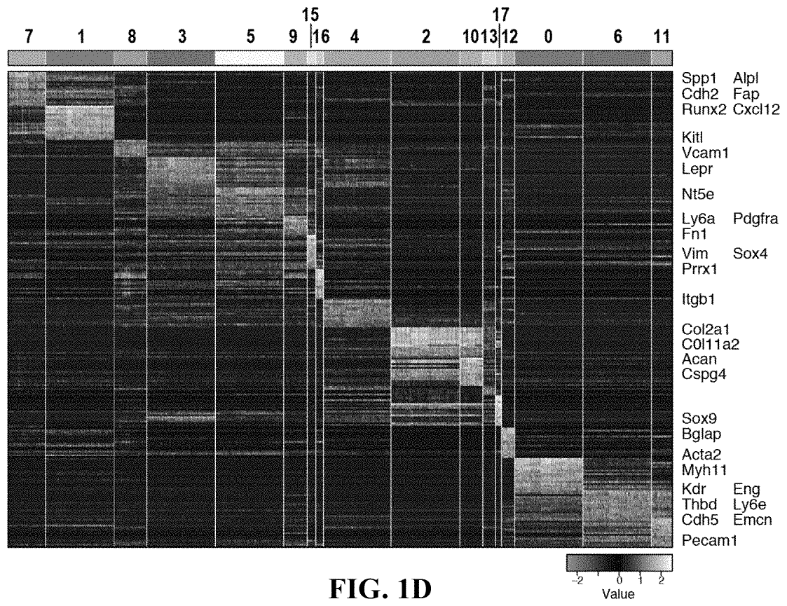

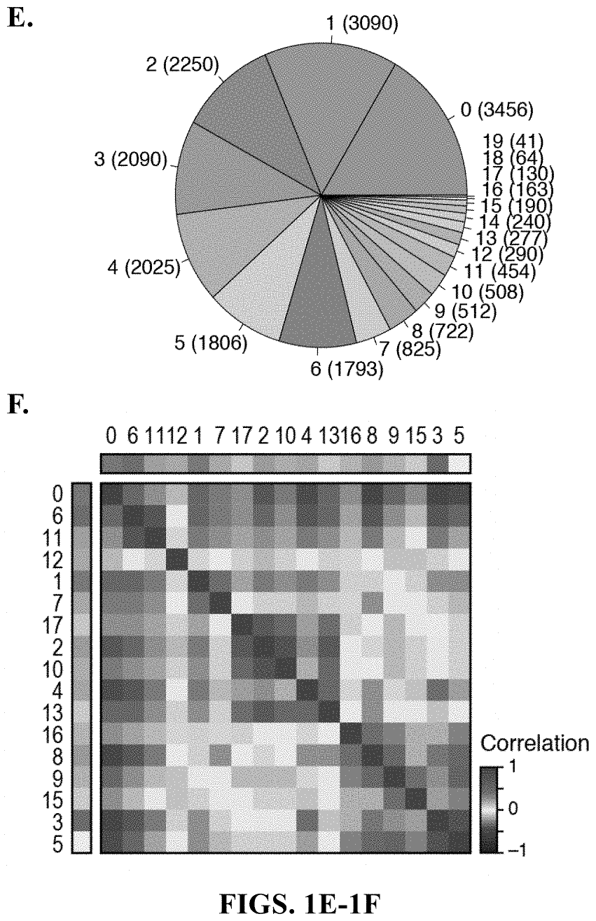

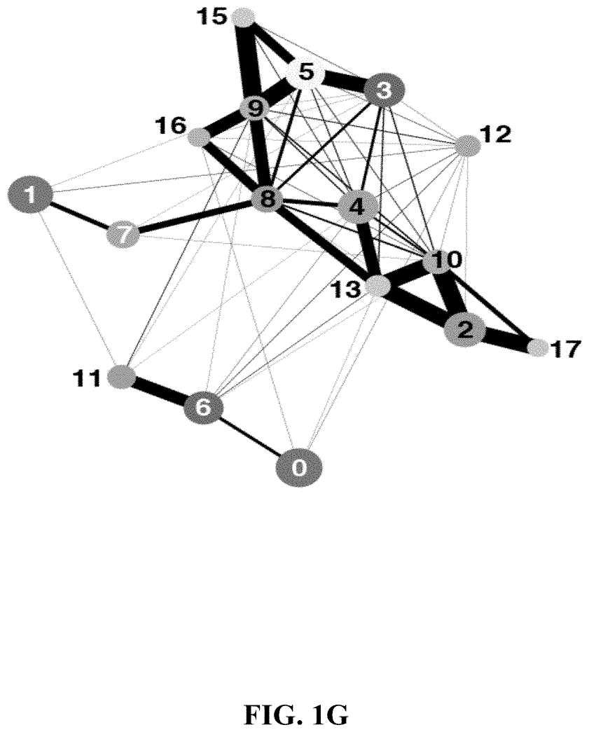

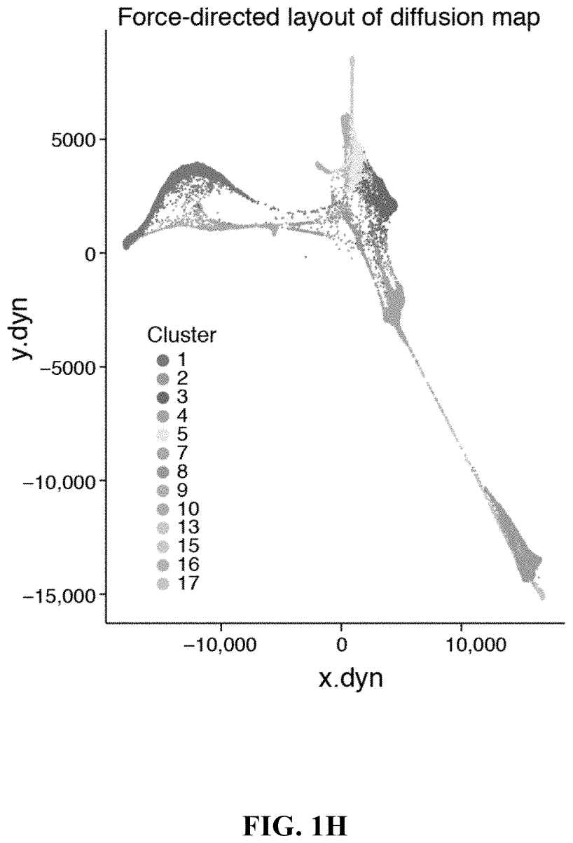

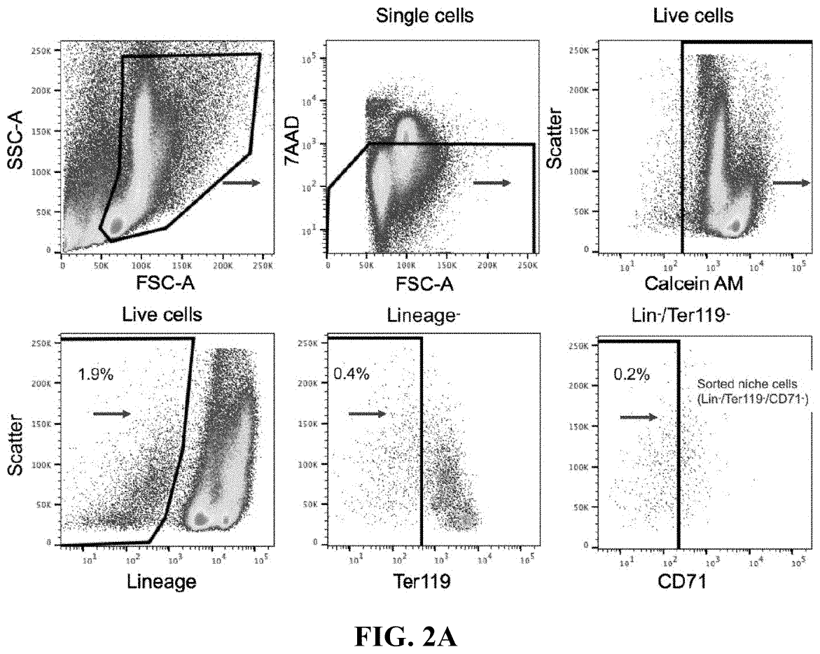

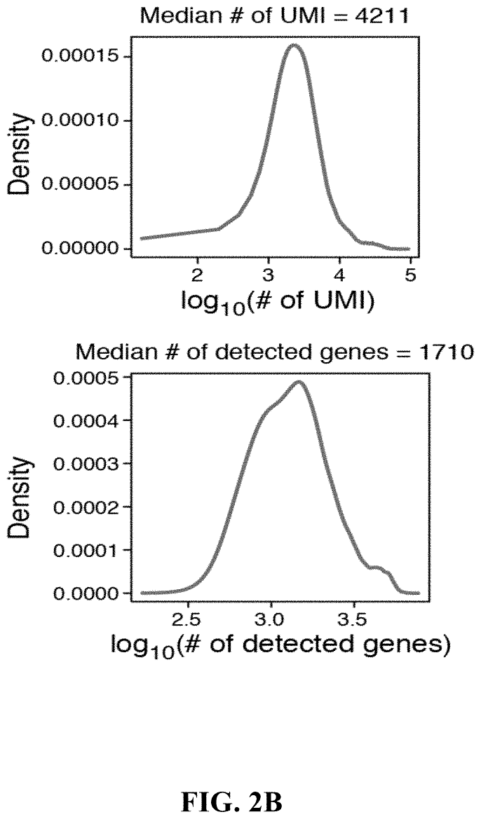

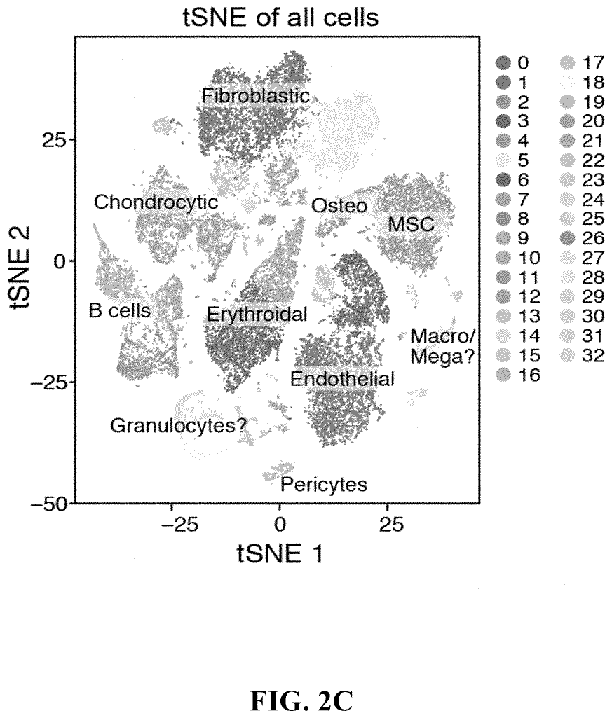

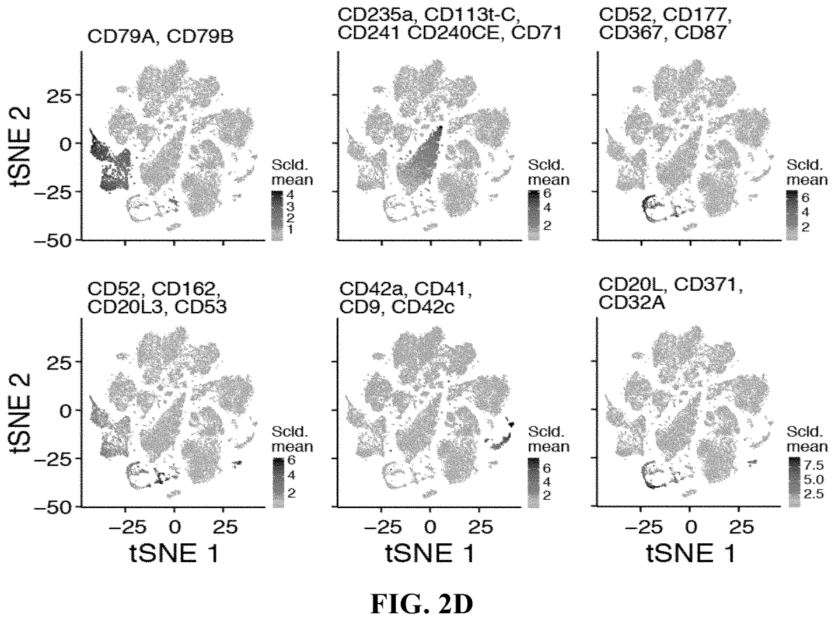

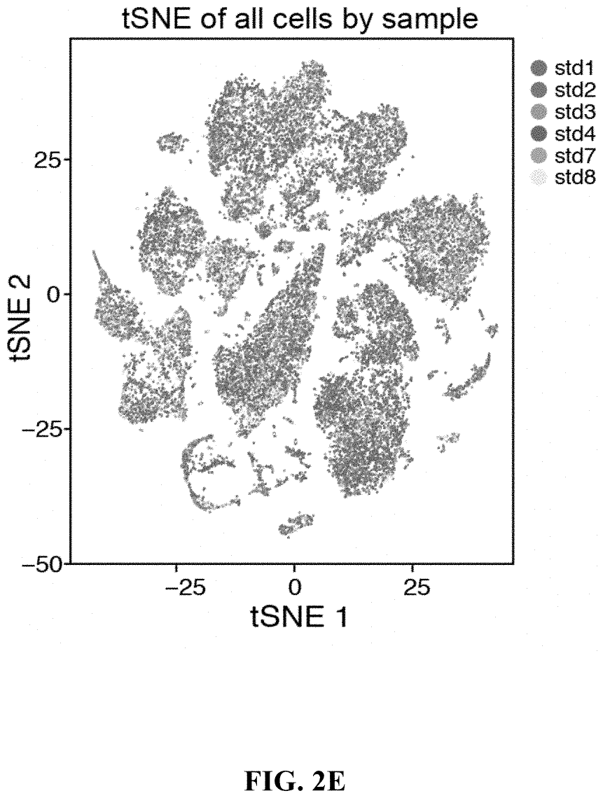

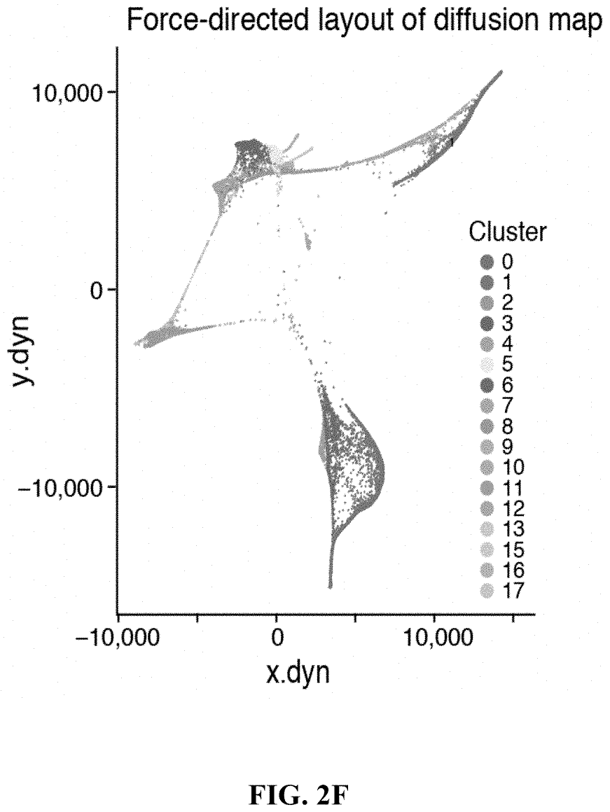

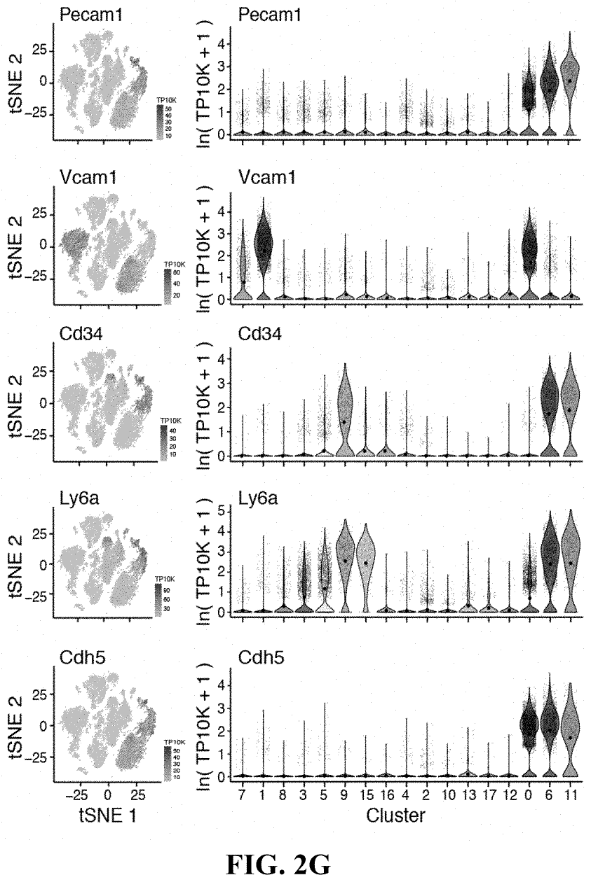

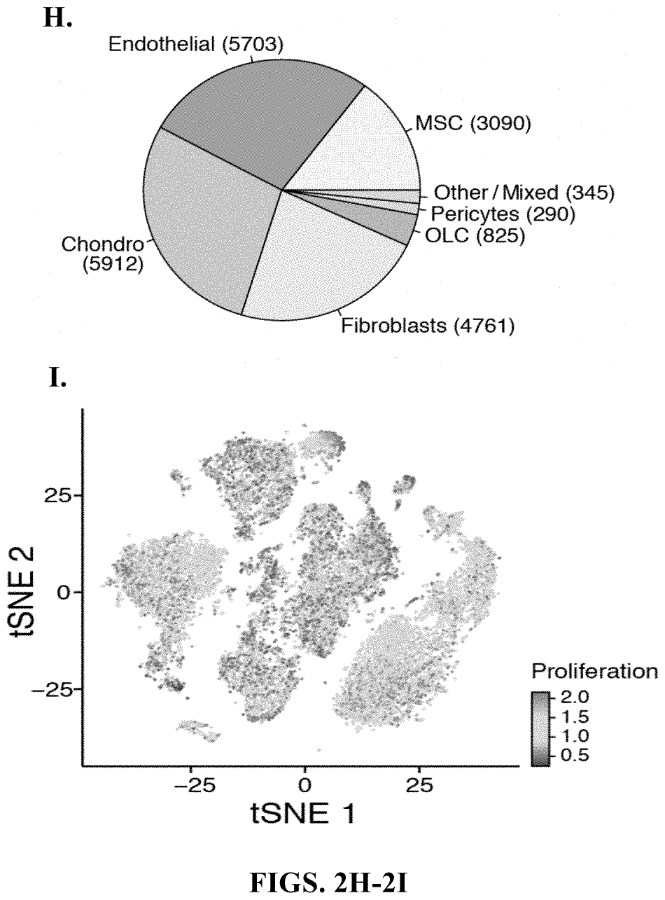

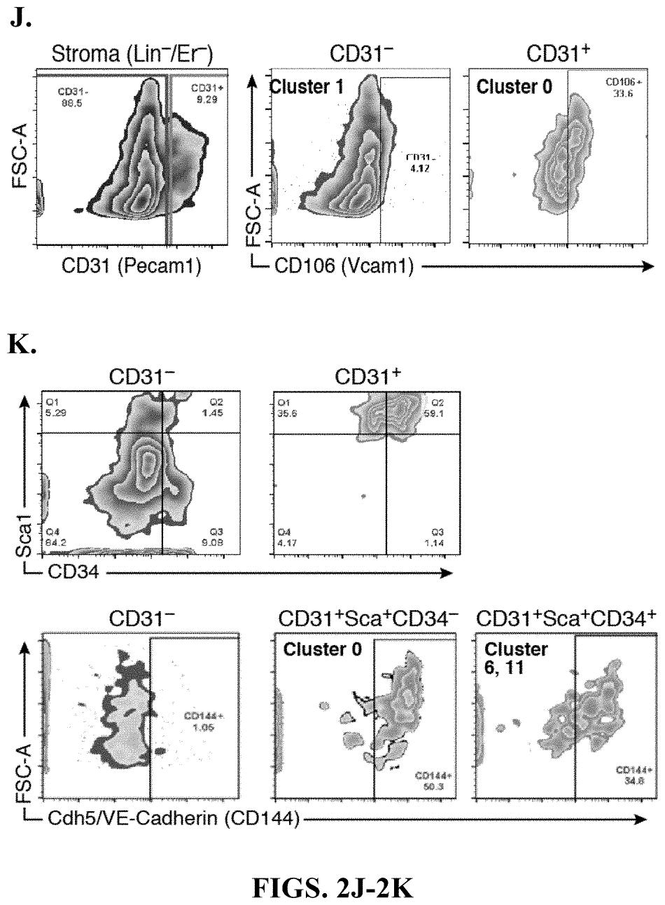

To explore the cellular composition of the stroma of the mouse bone marrow, non-hematopoietic bone marrow cells were profiled by scRNA-seq. Stroma cells were enriched from six C57Bl/6 mice at the age of 8-10 weeks by FACS (FIG. 1A), through selective depletion of hematopoietic cells and immune lineage cells (FIG. 2A), profiled them by droplet-based scRNA-seq, and retained 30,543 high quality profiles for further analysis (FIG. 2B). We partitioned the cells into 33 clusters by unsupervised clustering (FIG. 2C), which we annotated post-hoc, and distinguished subsets spanning 20,896 non-hematopoietic cells from 9,647 cells in hematopoietic clusters (FIG. 1B, FIGS. 2C-2D). Each of the non-hematopoietic clusters contained cells from all six mice (FIG. 2E).

Clustering of only the non-hematopoietic cells partitioned them into 17 clusters (FIG. 1B), spanning MSCs, OLCs, chondrocytes, fibroblasts, ECs and pericytes and possible transitional states (FIGS. 1C-1H, 2F-2H). In particular, there were: (1) MSCs (cluster 1, expressing Lepr (Ding and Morrison, 2013) and Cxcl12 (Greenbaum et al., 2013)); (2) two OLC subsets (Cluster 7 and 8, expressing osteopontin (Spp1) (Young et al., 1990)); (3) four chondrocyte subsets (clusters 2, 10, 13 and 17, annotated by expression of the chondrocyte markers, Aggrecan (Acan) (Doege et al., 1991) and Col2a1 (Chan et al., 1995)); (4) five fibroblasts subsets (Cluster 3, 5, 9, 15 and 16; expressing specific protein-1 (S100a4) (Strutz et al., 1995), collagens and extracellular matrix (ECM) proteins); (5) three EC subsets (Clusters 0, 6, 11; annotated by expression of the pan-endothelial marker Ve-cadherin (Cdh5) (Corada et al., 2001)); (6) one pericyte cluster (Cluster 12, expressing Acta2 (Armulik et al., 2011)); and (7) likely differentiating cells (Clusters 4 and 8, expressing markers of chondrocytes, osteoblasts and fibroblasts), indicating a potential differentiation pathway emanating from MSCs with distinct origin from other osteoblastic cells. Across subsets, chondrocytes, fibroblasts and OLCs had the highest proliferation status as estimated by expression of a cell cycle signature (FIG. 2I).

To help resolve differentiation relations between the cells we combined correlation of average profiles between the clusters (FIG. 1F), graph abstraction (Wolf et al., 2017b) (FIG. 1G), and diffusion map analysis (Schiebinger et al., 2017) (FIGS. 1H and 2F). This is discussed in greater detail elsewhere herein in the context of each lineage and differentiation path.

Two of the most abundant subsets were validated, MSCs and three EC subsets by FACS, showing that the four clusters can be partitioned prospectively by combining antibodies that labels ECs (CD31, Sca-1, CD34) or MSCs (CD106/Vcam1) (FIGS. 2J-2K) in combination with the antibodies we used previously to isolate stroma from immune (Lin−) and erythroid (Er−) cells (FIG. 2A).

### Example 3—HSC-Regulator Producing MSCs Form Four Subsets that Span a Differentiation Continuum

The prevailing model of MSCs in the bone marrow is that of a multipotent stem cell that can differentiate into bone cells, adipocytes and chondrocytes (Dominici et al., 2006), but their exact identity remain unclear. In particular, while many protein markers have been proposed and deployed (e.g., Cxcl12, LepR, Nes, NG2 (Cspg4) (Ding and Morrison, 2013; Ding et al., 2012; Kunisaki et al., 2013; Mendez-Ferrer et al., 2010; Sugiyama et al., 2006), CD73 (Nt5e), CD106 (Vcam1) (Mabuchi et al., 2013), CD105 (Eng) (Dominici et al., 2006), CD90 (Thy1) (Pittenger et al., 1999)), there is no single accepted combination and some may also be expressed by pericytes, ECs and chondrocytes (Kfoury and Scadden, 2015). It was hypothesized that more comprehensive transcriptional profiles will better identify subsets and relate them to legacy markers.

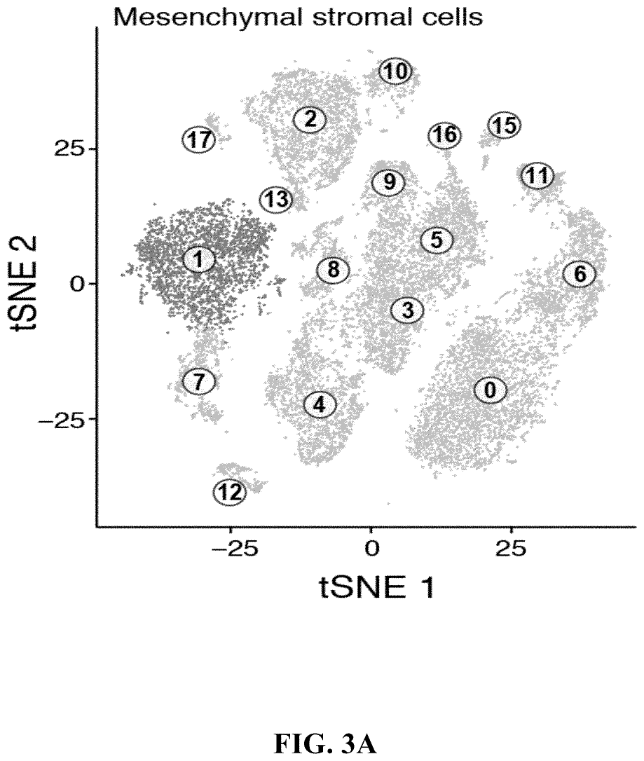

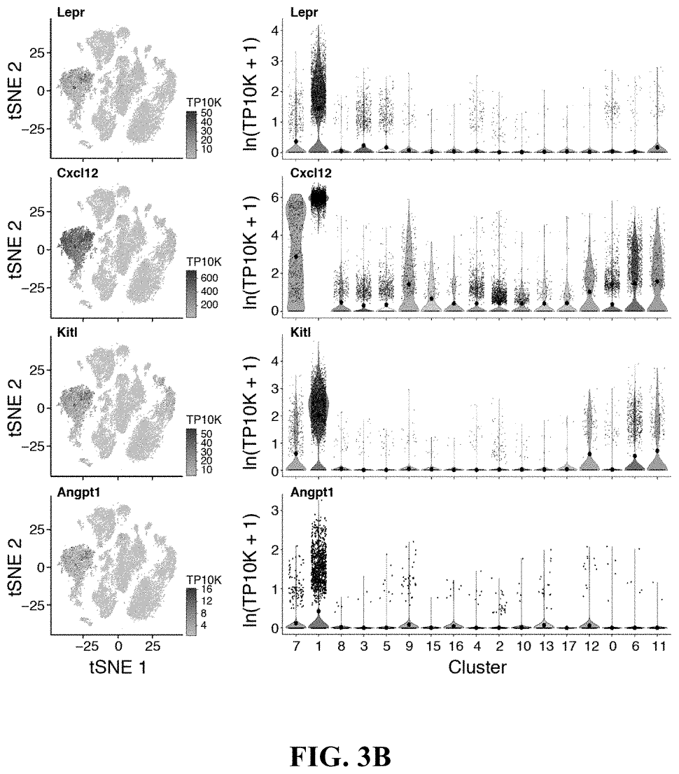

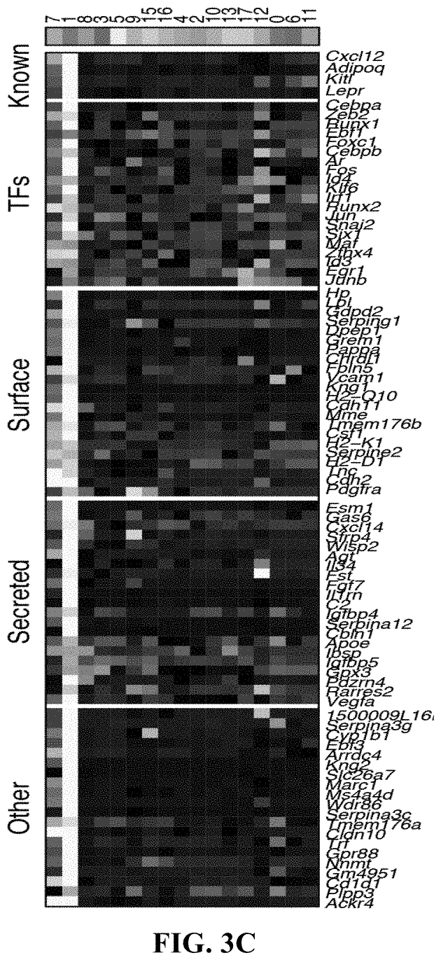

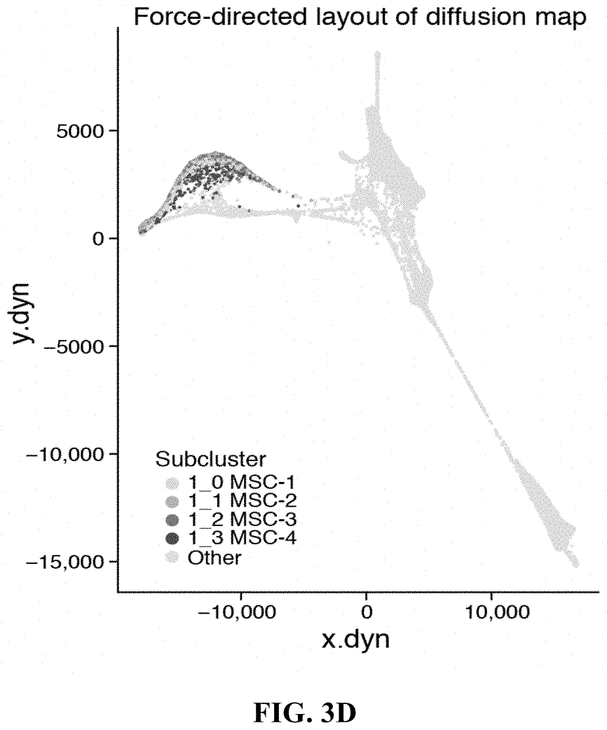

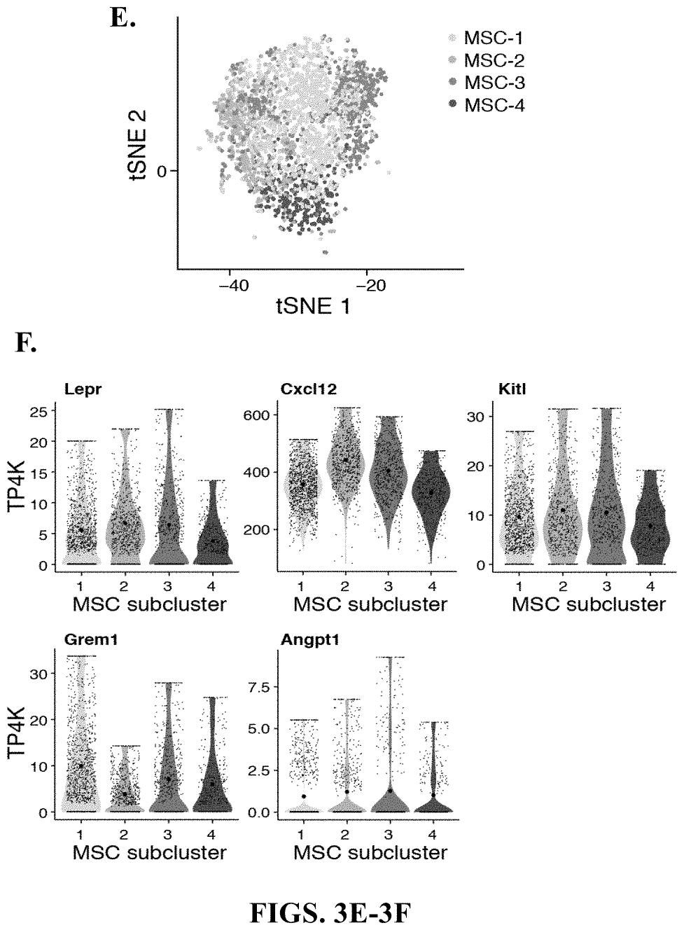

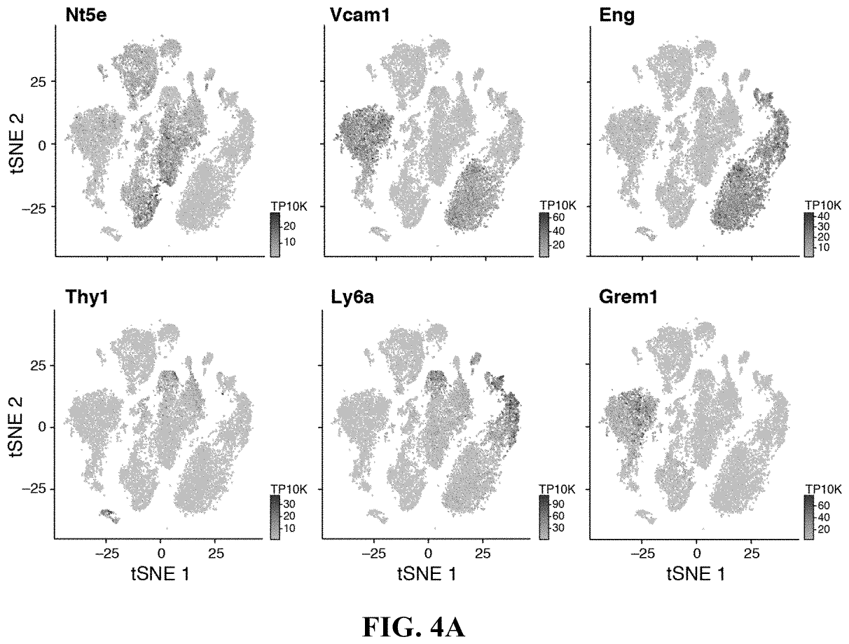

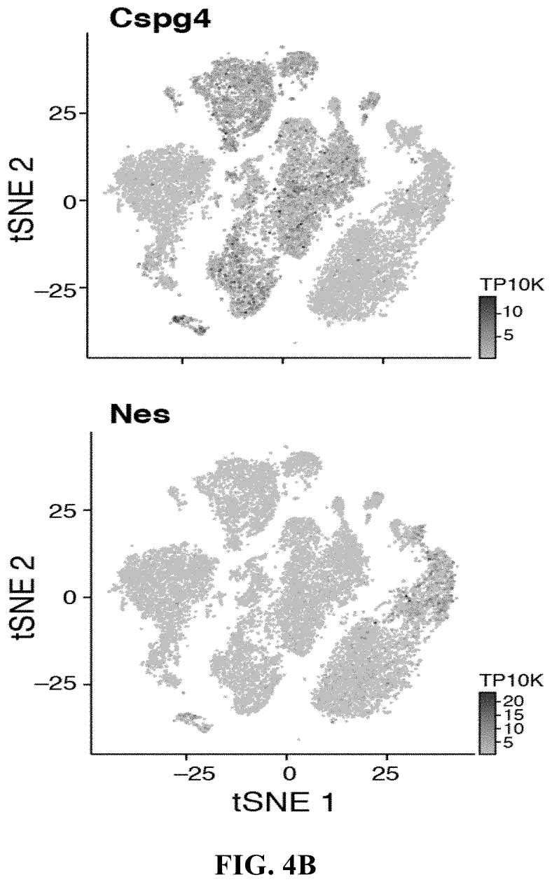

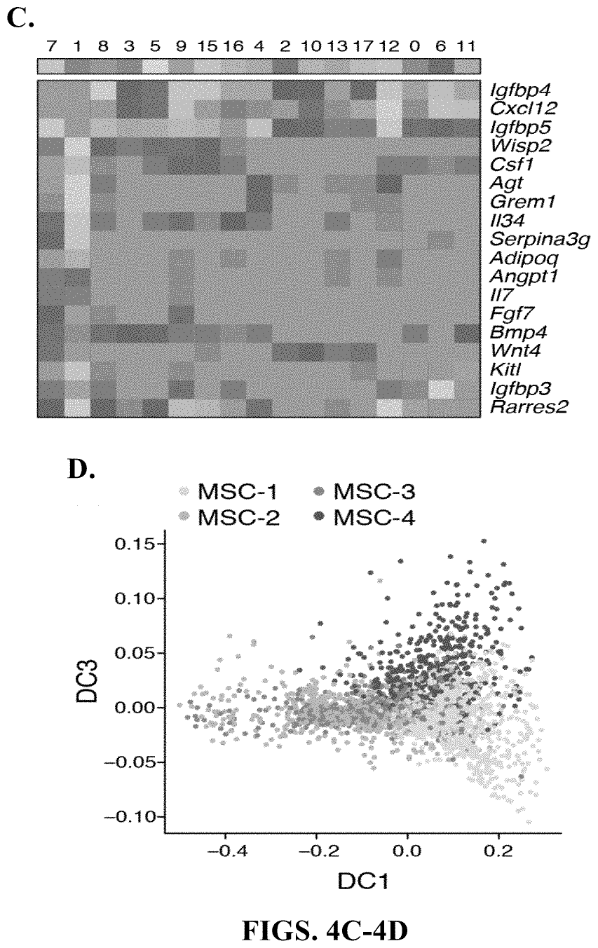

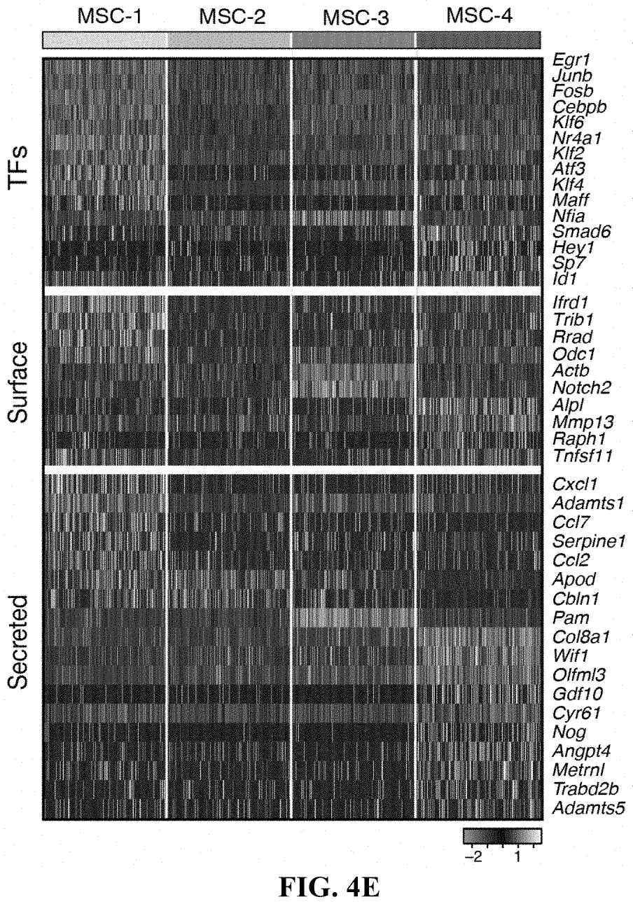

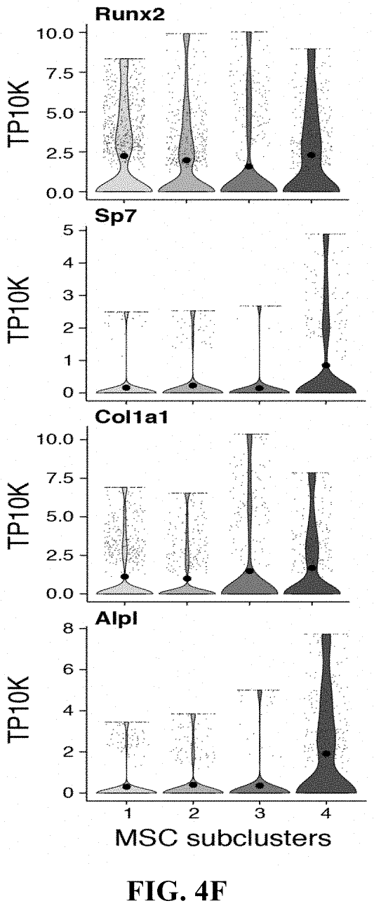

Cluster 1 was annotated as LepR⁺ MSCs/CAR cells (Ding et al., 2012; Greenbaum et al., 2013; Omatsu et al., 2010; Sugiyama et al., 2006). First, the cells highly expressed Lepr, a perivascular MSC marker (Ding et al., 2012; Morrison and Scadden, 2014), and Cxcl12, Kitl and Angpt1 (FIG. 3B,C), key niche factors produced by bone marrow niche cells. Notably, Cxcl12, Kitl and Angpt1 were more lowly expressed in additional clusters, especially a subset of OLCs (cluster 7, FIG. 3B), which were contiguous with cluster 1 in the inferred differentiation maps (FIGS. 1G-1H), suggesting continuous transitions in niche functions on the MSC-OLC differentiation path. The cells expressed additional MSC markers (Nt5e, Vcam1 and Eng) as well as Grem1, a BMP antagonist, a marker of mouse skeletal stem cells with bone, cartilage and reticular marrow stromal cell potential (Worthley et al., 2015) (FIG. 4A). Other previously proposed MSC markers such as Thy1, Ly6a (Sca-1) and the perivascular MSC markers NG2 (Cspg4) and Nestin (Nes) (Kunisaki et al., 2013; Mendez-Ferrer et al., 2010) were not expressed in cluster 1 (FIG. 4A), but in other, non-MSC cells (FIG. 4B).

Within cluster 1 MSCs, four major subsets were further distinguished by sub-clustering and diffusion trajectory analysis (Angerer et al., 2016) (FIGS. 3D-3E, and 4D-4E). While Lepr, Kitl and Angpt1 were comparably expressed in all four sub-sets, they were distinguished by the expression of Cxcl12, Grem1 and multiple other genes (FIGS. 3F and 4D). In particular, one subset (MSC-4) had high expression of osteolineage cell-specification genes (Runx2, Sp7 (osterix), Col1a1 and Alpl (Huang et al., 2007; Nakashima et al., 2002)) (FIGS. 4E-4F), suggesting differentiation towards the osteoblastic lineage, as supported by continuous transition between cells from the MSC and OLC-1 clusters in differentiation trajectories (FIGS. 1G-1H). Indeed, OLCs, that include osteoprogenitors and mature osteoblasts, can be derived from MSCs (Kfoury and Scadden, 2015).

### Example 4—Inferred Lineage Relations Suggest Two Osteolineage Subsets of Different Differentiation Origins and Distinct Hematopoietic Support Potential

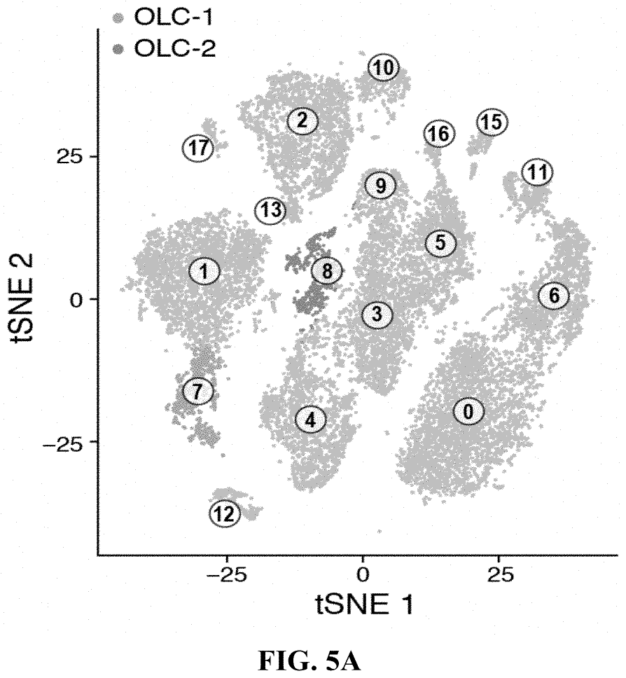

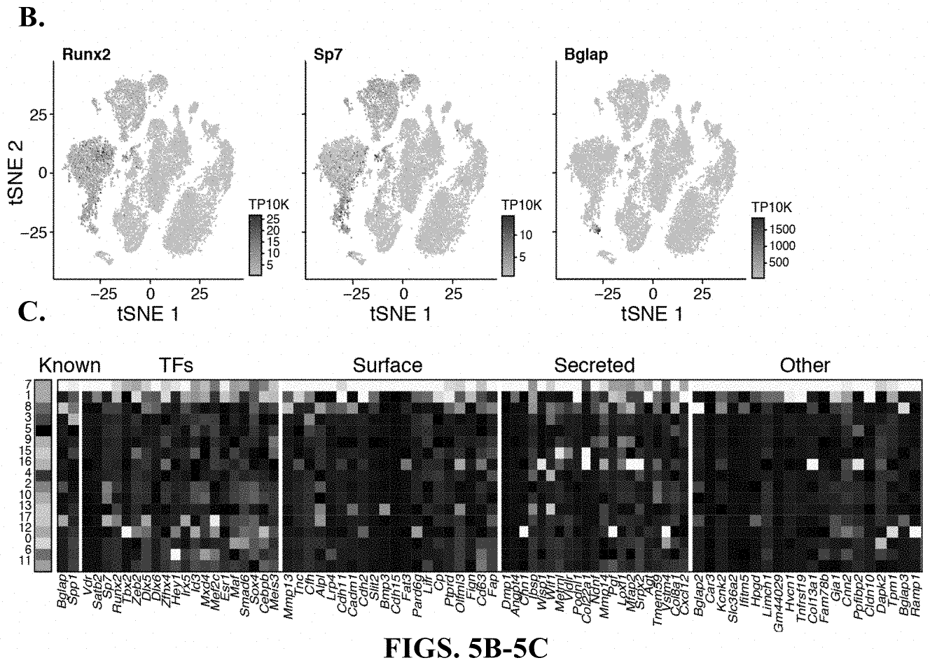

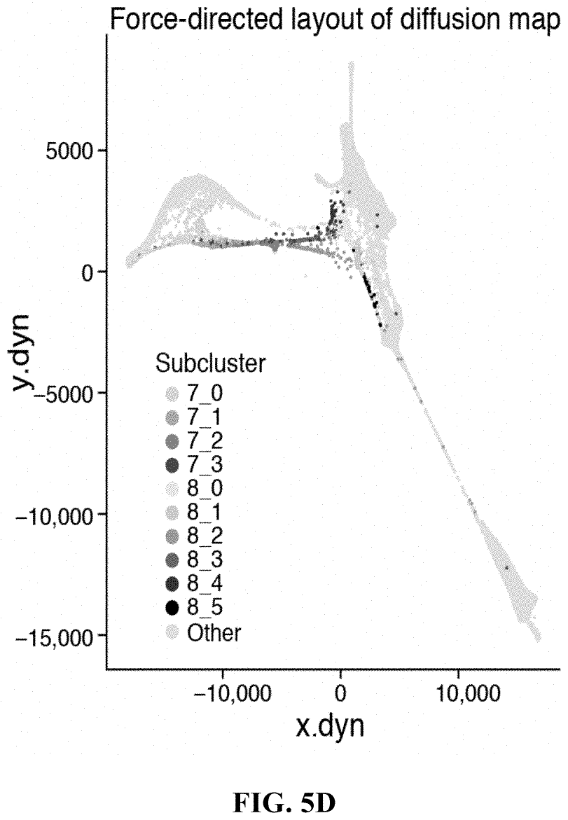

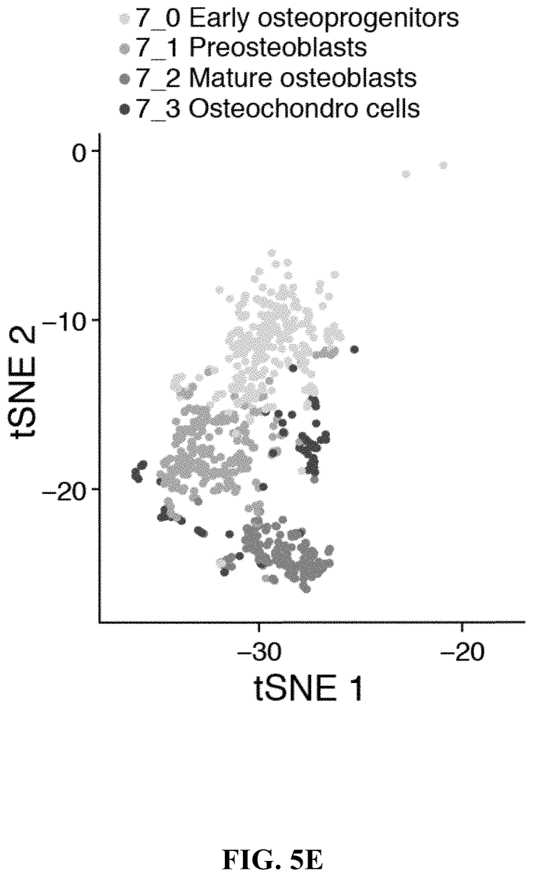

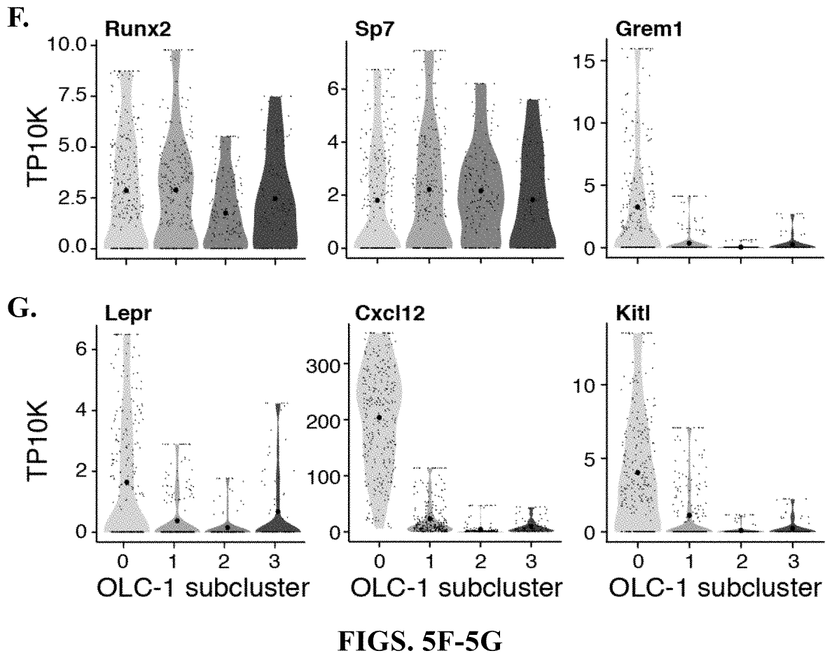

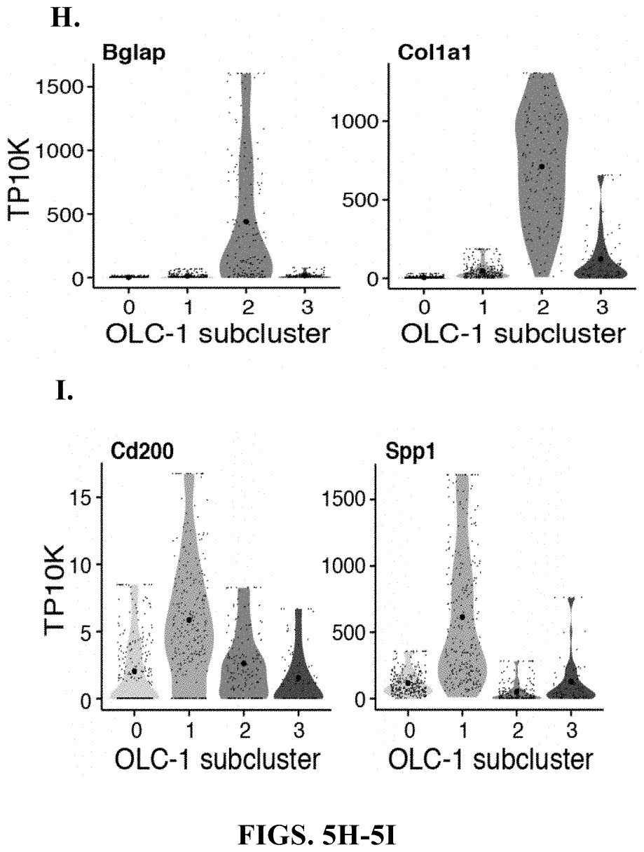

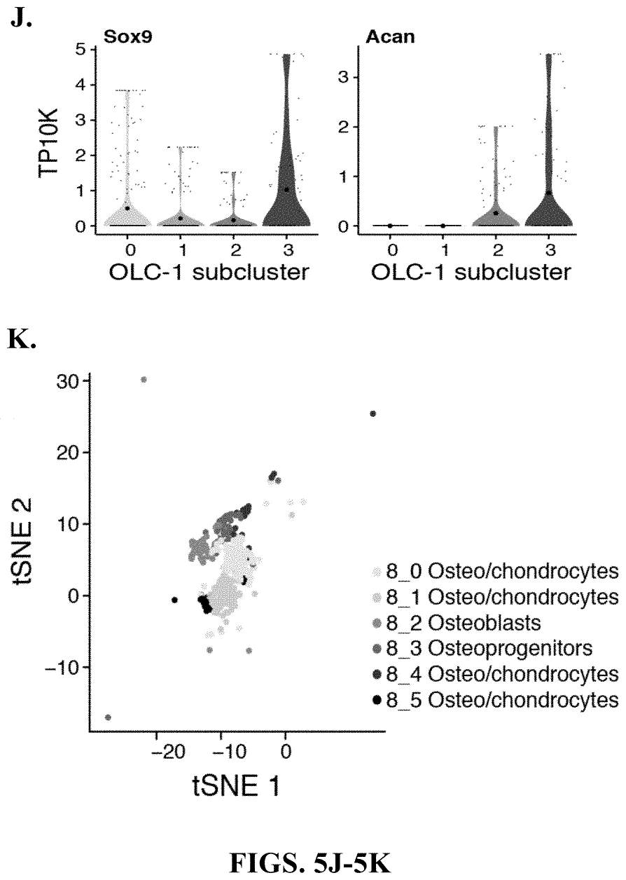

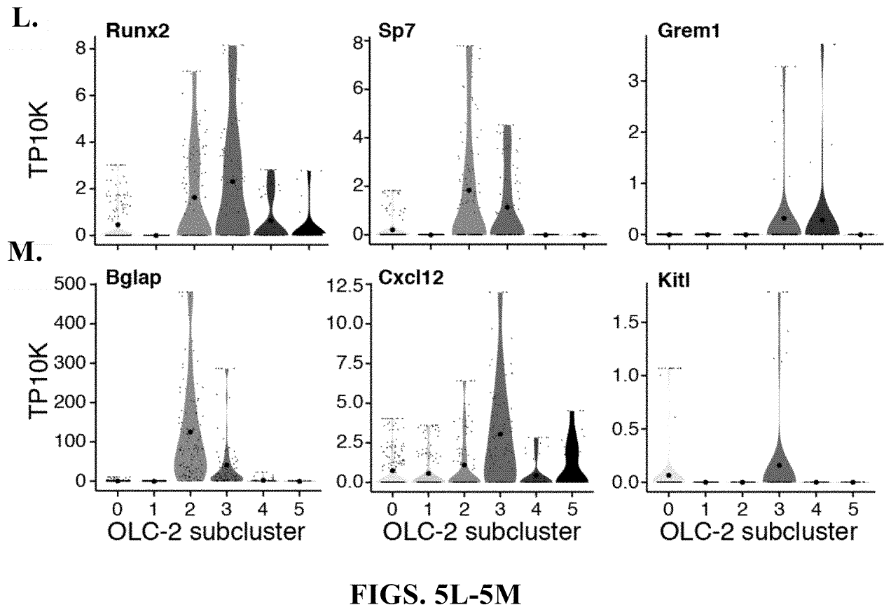

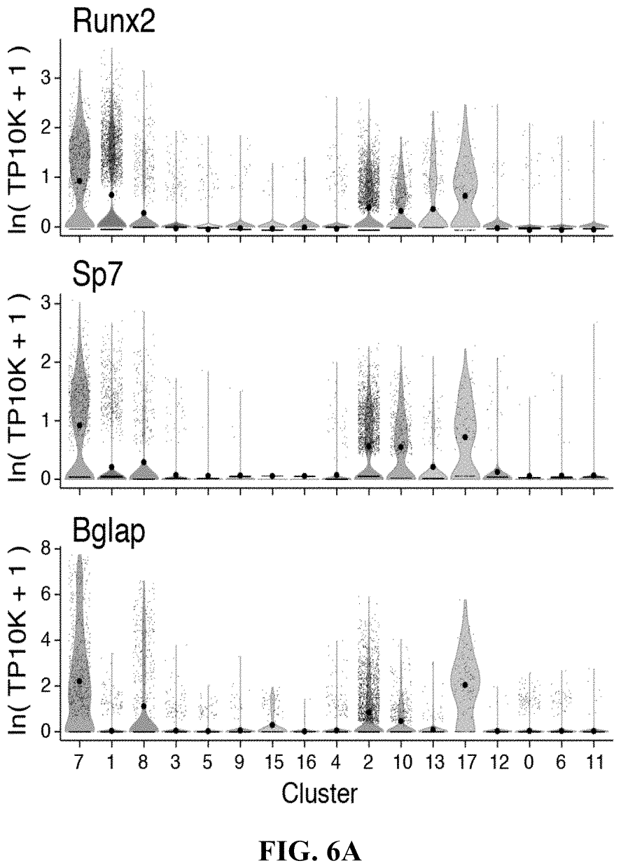

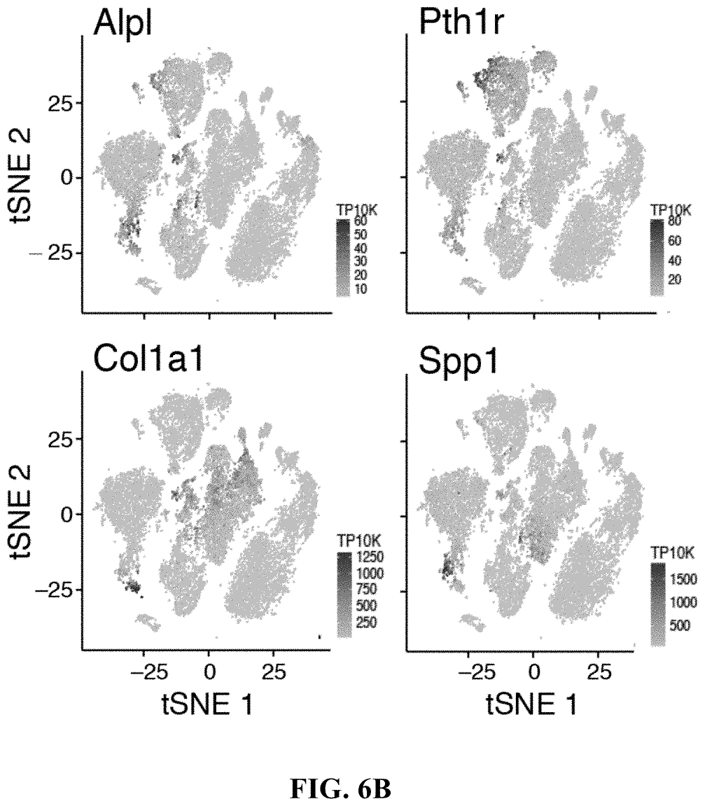

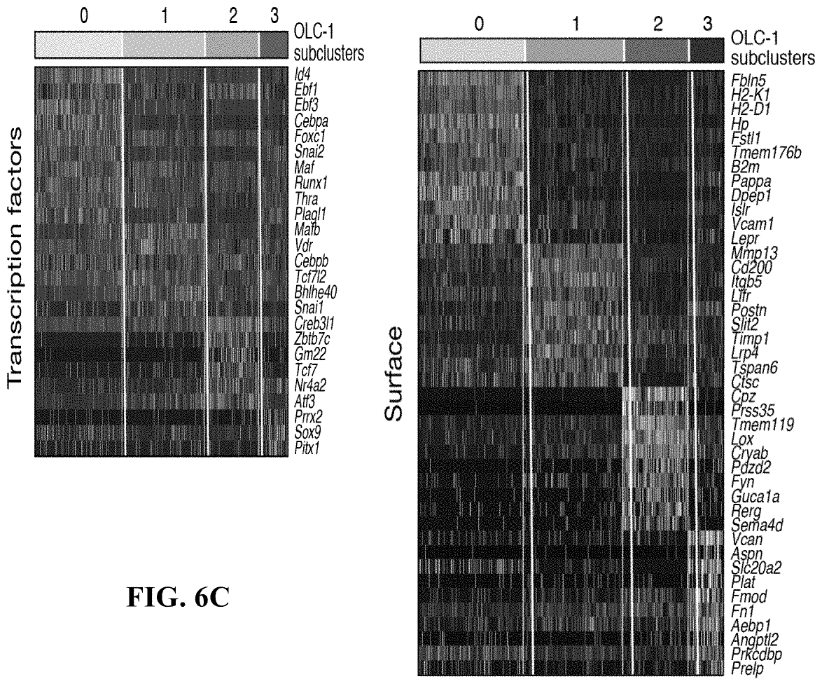

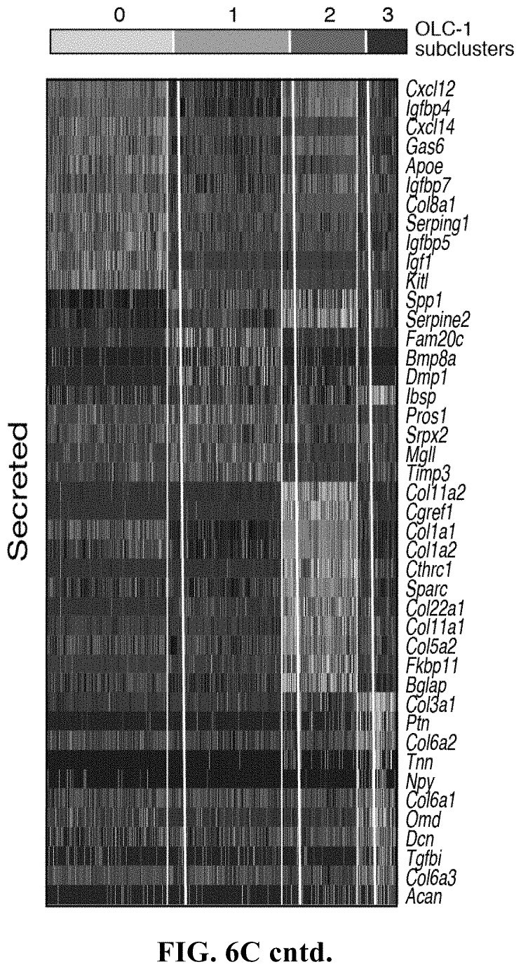

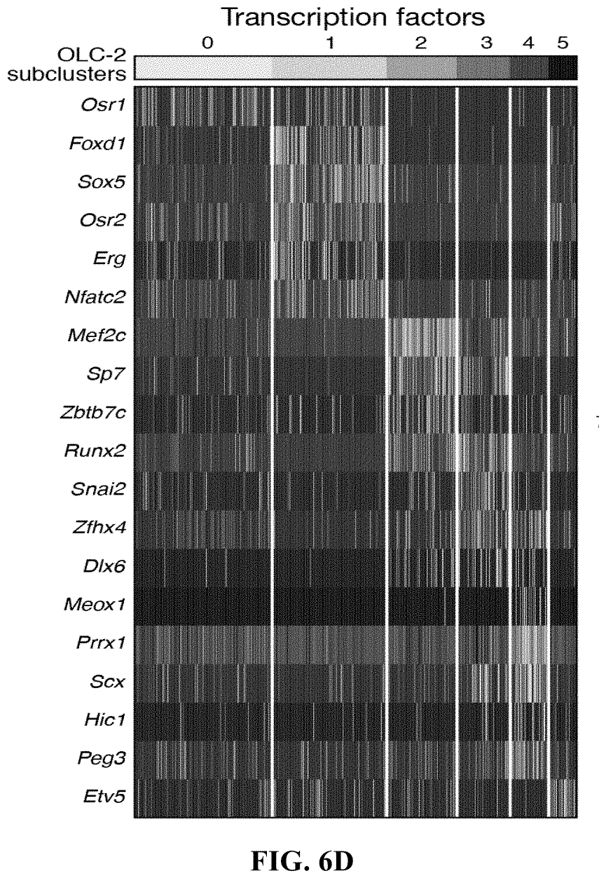

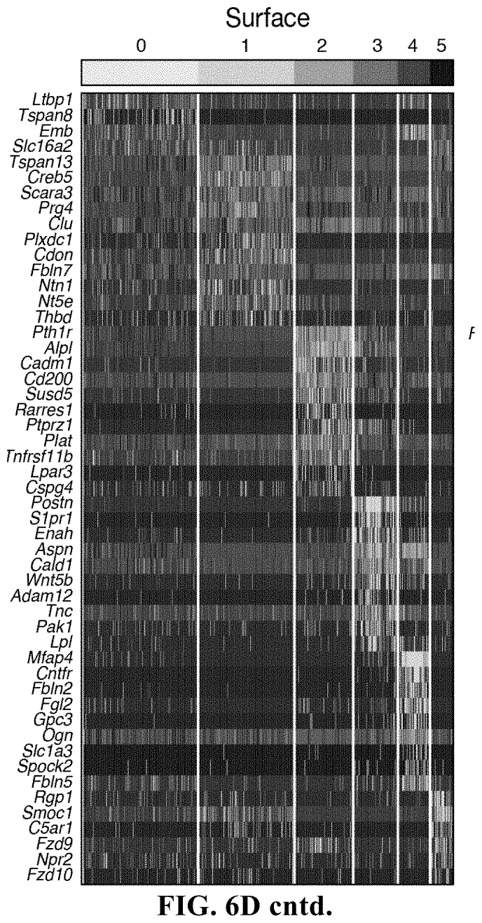

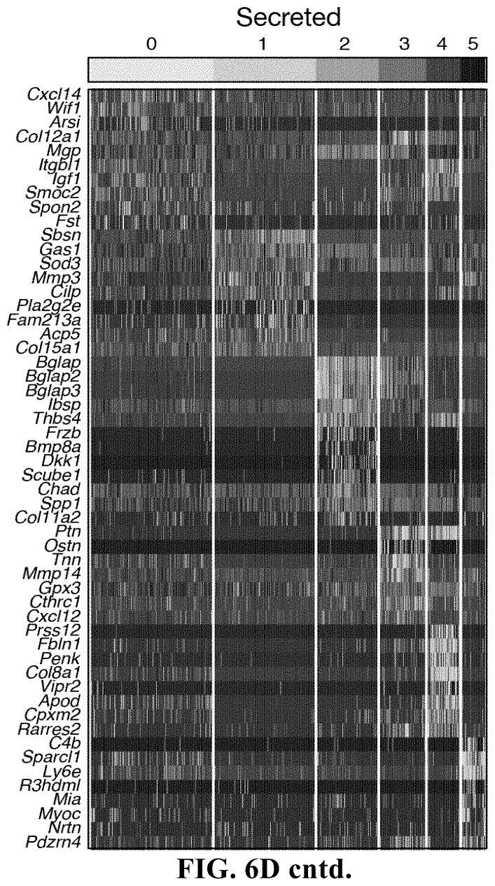

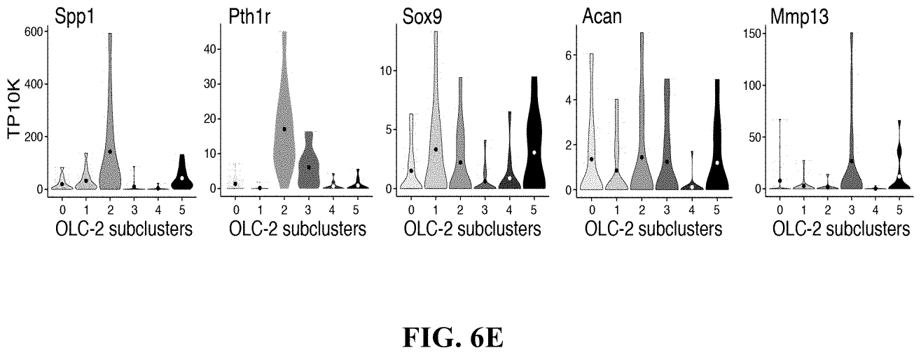

To further analyze osteolineage differentiation, cells from the MSC-4 subsets were focused on, along with cluster 7 (OLC-1) and 8 (OLC-2) cells (FIG. 5A) that express the osteoblast maturation marker Bglap (Delmas et al., 1986), and together form a continuum in the diffusion map (FIG. 1H). All three clusters differentially express Runx2, the master regulator TF controlling the commitment of MSCs to OLCs (Huang et al., 2007), and both OLC-1 and OLC-2 further express Sp7, its downstream target TF, which together are required for both early and late differentiation (FIGS. 5B-5C and 6A). Furthermore, Bglap is expressed in a subset of the clusters' cells (FIG. 5B), along with other osteoblastic maturation makers (Alp1, Col1a1, Spp1 and Pthlr (Huang et al., 2007)) (FIGS. 5C and 6B).

Whereas MSC-4 reflects committed osteolineage MSCs, the cells in OLC-1 span a continuum of osteoblastic differentiation as indicated by diffusion map (FIGS. 5D-5E) and differential gene expression (FIG. 6C) analyses, including, in order: (1) osteolineage committed early progenitors that were in a cell transition-state from MSC-4 (FIGS. 3E and 5D-5E), expressing Runx2, Sp7, Grem1, Lepr, Cxcl12 and Kitl (FIGS. 5F-5G and 6C) but not osteoblast maturation markers (Bglap and Col1a1; FIG. 511); (2) late osteoprogenitors cells (e.g. preosteoblasts), highly expressing the skeletal stem cell and osteoprogenitor marker CD200 (Abdallah et al., 2015; Chan et al., 2015) and Spp1 (FIG. 5I) a marker of later stages of osteoblastic differentiation (Huang et al., 2007), but lowly expressing osteoblast maturation markers (FIG. 5H); and (3) mature osteoblasts, expressing Bglap and Col1a1 (FIG. 5H). In addition, the cluster included (4) chondrocytes, which are likely transitioning into osteoblastic cells, through endochondral ossification (Yang et al., 2014; Zhou et al., 2014b). These expressed low levels of Sox9, a TF essential for chondrocyte differentiation (Foster et al., 1994; Wagner et al., 1994), and Acan, a chondrocyte marker gene (Doege et al., 1991) (FIG. 5J).

Remarkably, the OLC-2 cells highlighted a distinct set of OLCs from those found in a continuum from MSCs. Along the continuum spanned by OLC-2 cells (FIGS. 5D-5K and 6D), it was observed that: (1) Osteoprogenitors, expressing Runx2, Sp7, and Mmp13, the matrix metalloproteinase that regulates the calcification and degradation of cartilage during the endochondral ossification process (Nishimura et al., 2012) (FIGS. 5L and 6E), but low levels of Bglap and Sox9, (FIGS. 5M and 6E); (2) Osteoblasts, expressing OLC markers (Runx2, Sp7, Spp1, Bglap and Pth1r, FIGS. 5L-5M and 6E); and (3) osteo/chondrocytes that were in the process of being ossified into bone (Yang et al., 2014; Zhou et al., 2014b). Notably, only OLC-1 cells express key hematopoietic regulatory cytokines (albeit subcluster 8_3 in OLC-2 express relatively low levels Cxcl12) (FIG. 5M), suggesting that there are two osteolineage subsets of distinct differentiation origins and with distinct hematopoietic support potential.

### Example 5—Characterization of Chondrocyte Cell Differentiation

Next the differentiation of chondrocytes was examined. Cartilage is formed by chondrocytes that are derived from condensed MSCs that differentiate into chondrocyte progenitors. Cartilage development occurs at the growth plate where chondrocytes transition between proliferating and resting states, differentiate to hypertrophic chondrocytes, which in turn terminally lose their capacity to proliferate, and finally are ossified into bone (Nishimura et al., 2018).

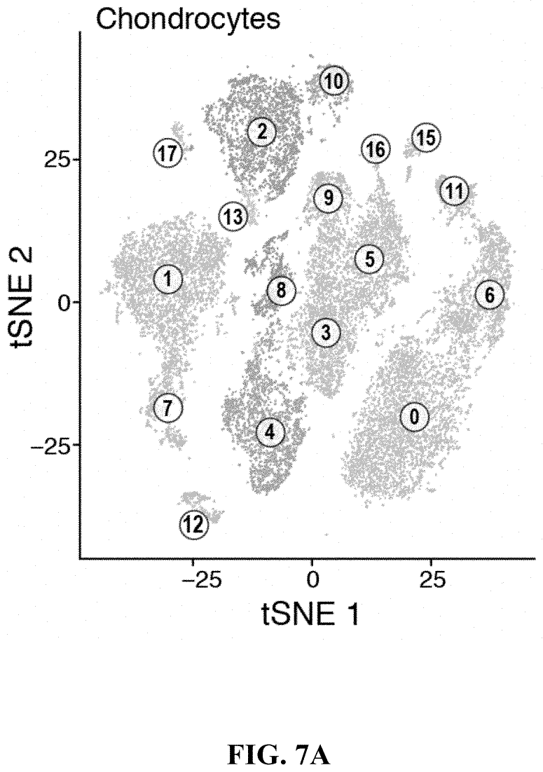

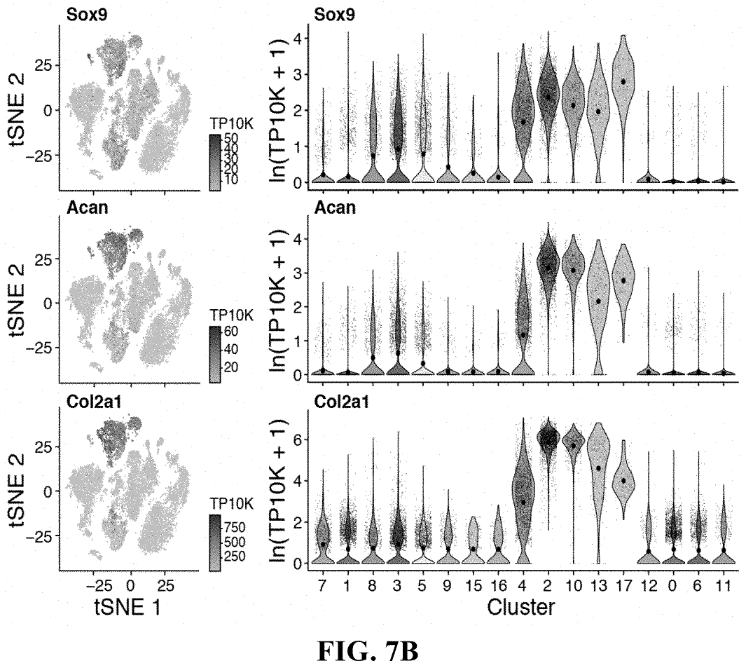

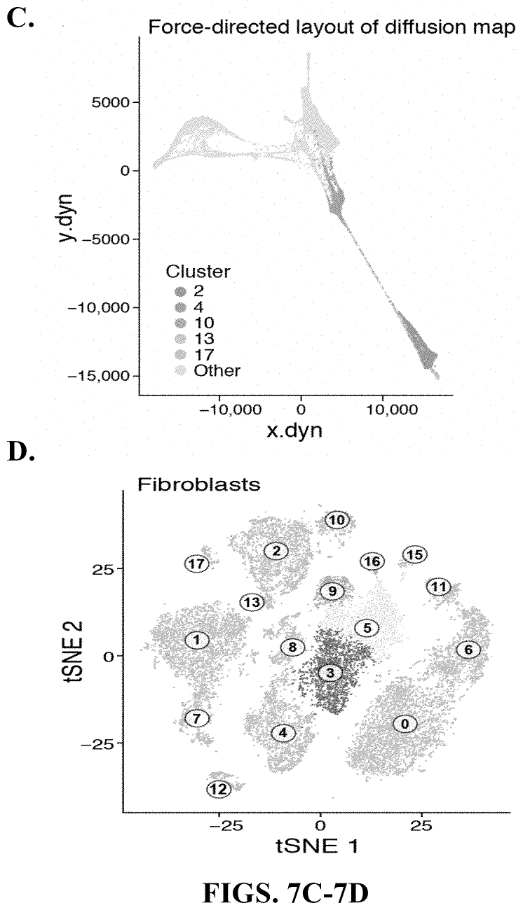

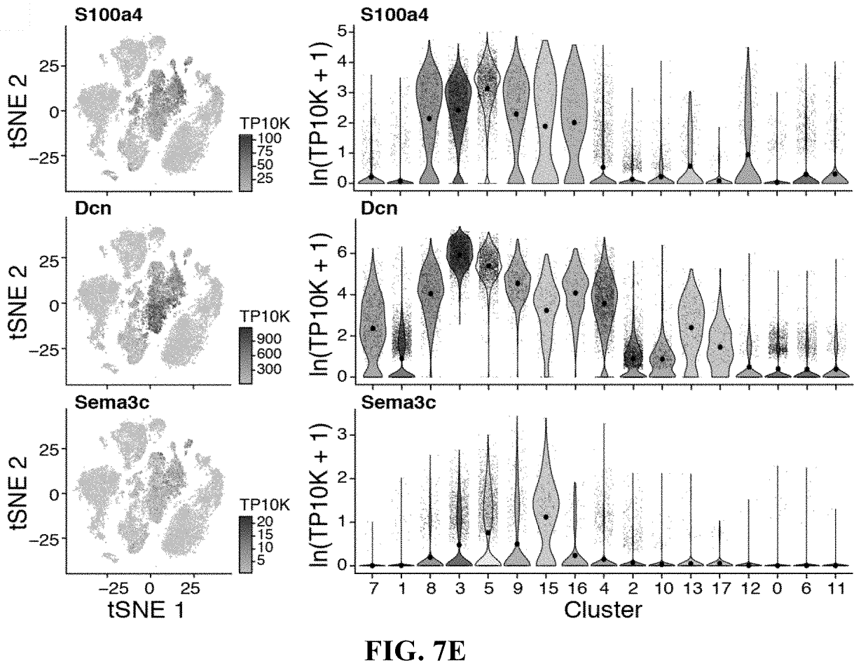

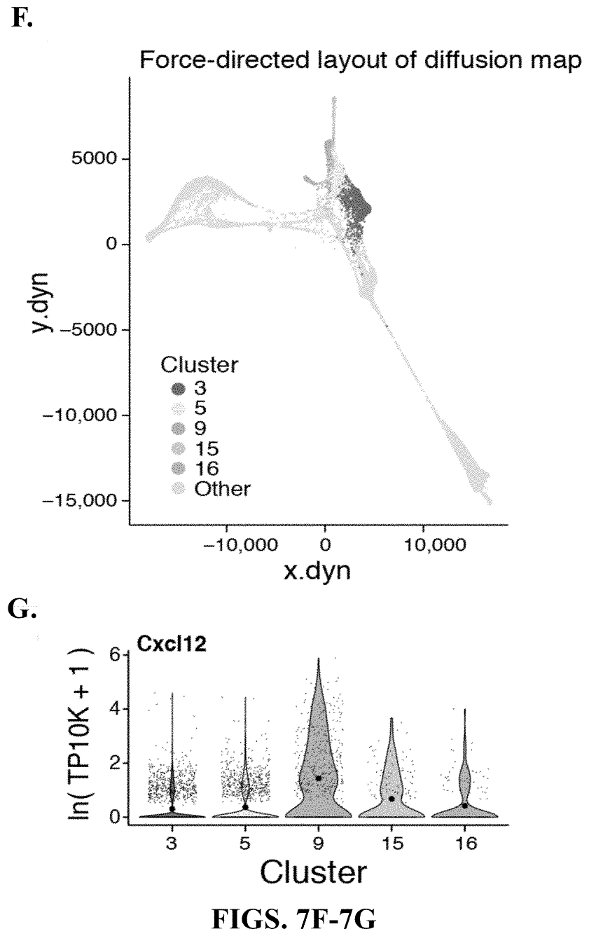

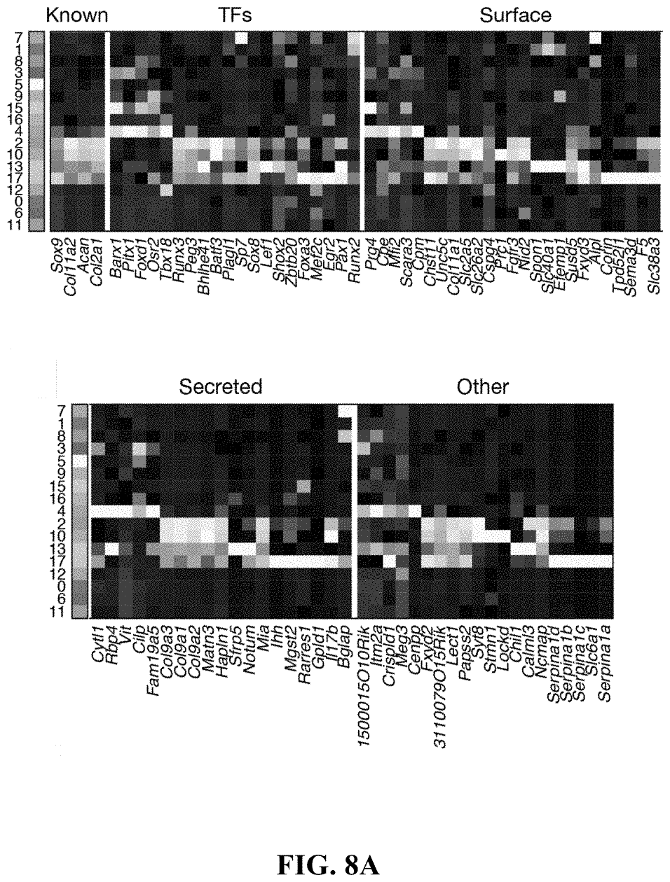

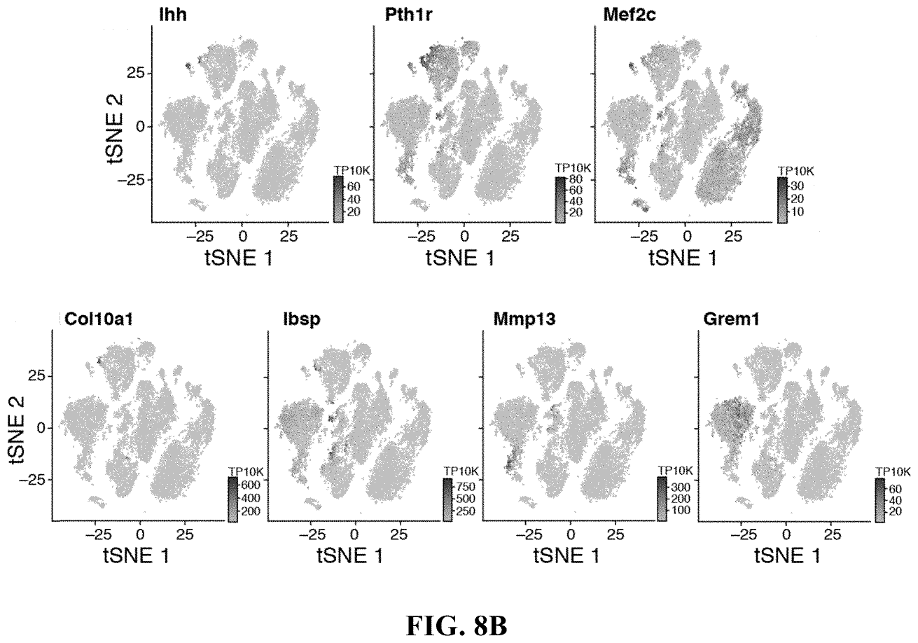

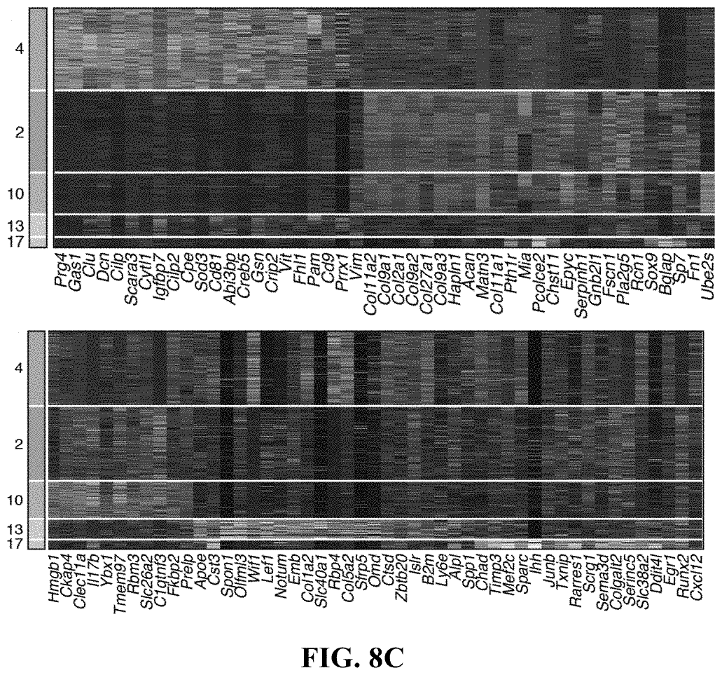

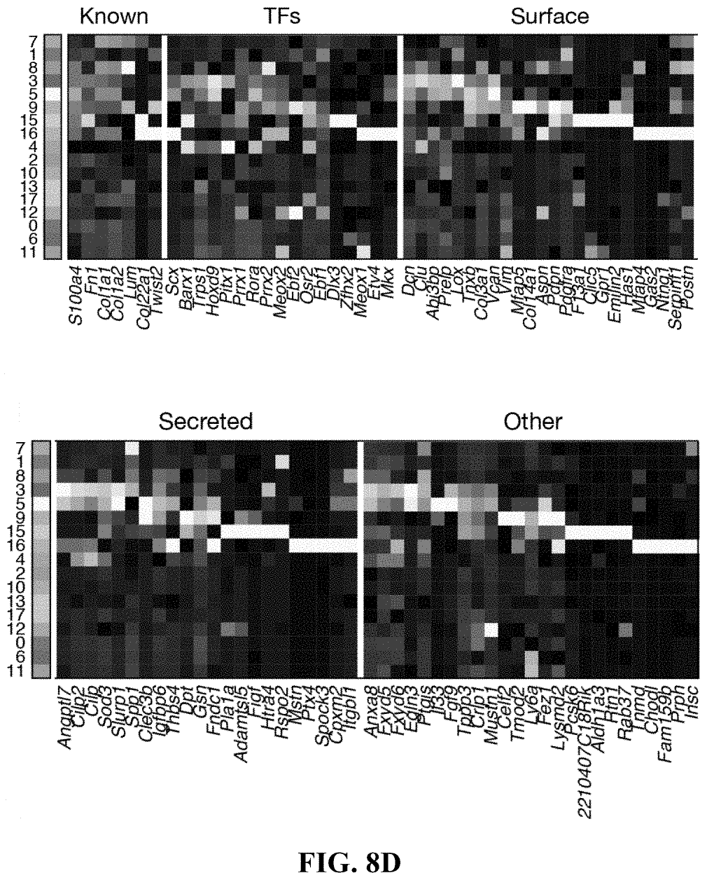

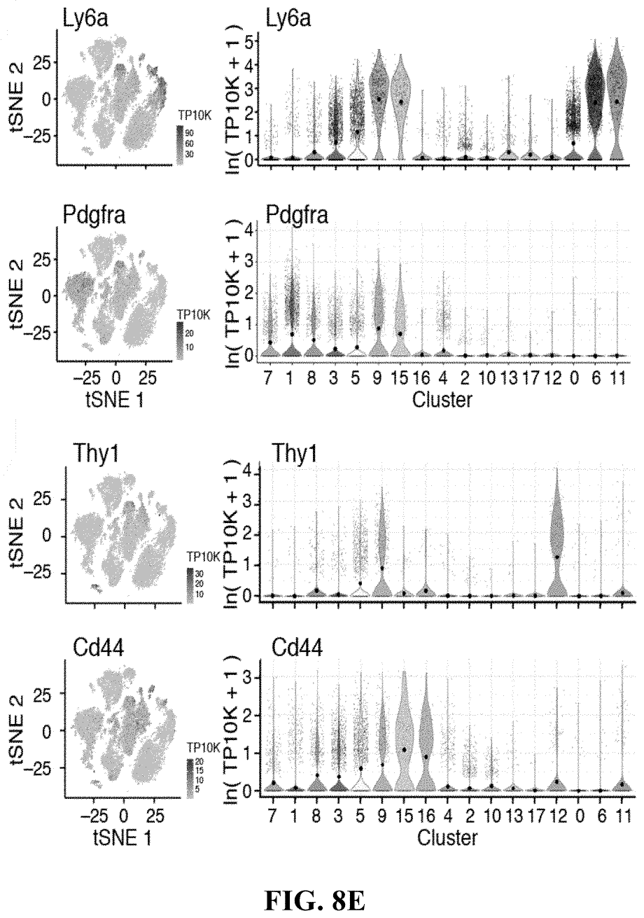

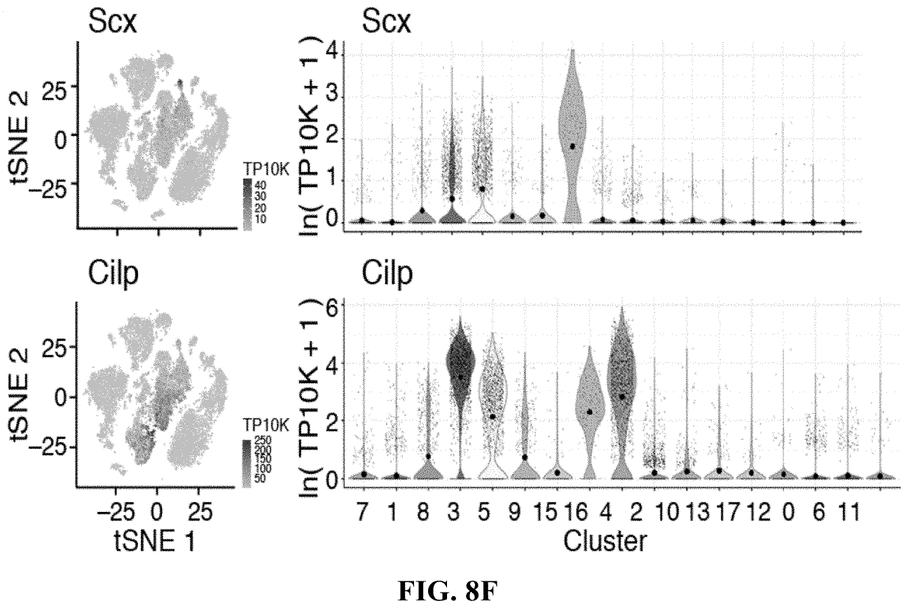

Cells in five clusters (Clusters 2, 4, 10, 13, 17) all expressed the chondrocyte lineage genes Sox9, Acan and Col2a1 (FIGS. 7A-7B), formed a sequence in the overall diffusion map (FIG. 7C) and distinguished (1) Proliferating and resting chondrocytes (Cluster 10), expressing Col2a1 (Inada et al., 1999) (FIGS. 21 and 7A-7B); (2) prehypertrophic chondrocytes that started the maturation process (Clusters 2 and 17), co-expressing Ihh, Pthlr (Vortkamp et al., 1996), Mef2c (Arnold et al., 2007) (FIGS. 7A and 8A-8C), (3) hypertrophic chondrocytes (Cluster 2), co-expressing Runx2, Ihh, Mef2c and Col10a1 (FIGS. 7A and 8A-8C) (Arnold et al., 2007); as well as (4) chondrocyte progenitor cells that are in transition into cluster 8 (Cluster 4) (FIGS. 1H and 7C), expressing Grem1 (FIG. 8B), OLC markers (Runx2, Sp7, Alp1, Spp1) (FIGS. 5B and 6B), and chondrocyte markers (Sox9, Acan; FIG. 7B).

### Example 6—a Bone Marrow Derived Fibroblast Subset Expresses Hematopoietic Niche Regulators

Fibroblasts are cells of mesenchymal origin that are ubiquitous in the bone marrow, and consist of phenotypically and functionally distinct subpopulations. Currently, fibroblasts were predominantly identified by Fn1, Pdgfra, Fibroblast Specific Protein-1 (S100a4) and Acta2 expression (Kalluri, 2016). Due to the paucity of distinctive markers and similar morphology and phenotypes, they are commonly confounded in the bone marrow with MSCs and perivascular cells, limiting the accuracy of functional studies (Soundararaj an and Kannan, 2018). It was hypothesized that we can better define the precise identity and function of bone marrow fibroblasts by their transcriptional profiles.

Five fibroblasts clusters (3, 5, 9, 15, 16, FIG. 7D) all highly expressed the fibroblast specific genes Fibronectin-1 (Fn1), Fibroblast Specific Protein-1 (S100a4), Decorin (Dcn), and Semaphorin-3C (Sema3c) (FIGS. 7D-7E), lowly expressed the chondrocyte genes Sox9, Acan, and Col2a1 (FIG. 7B), and were distinguishable from both MSCs and pericytes. Cells in distinct clusters expressed different collagens, indicating specialized roles in connective tissue formation (Gelse et al., 2003) (Col1a1 and Col1a2 in clusters 3, 5 and 9; Col22a1 in clusters 15 and 16; FIG. 8D).

Fibroblasts-1 (cluster 9) and 2s (cluster 16) had MSC characteristics and some may provide niche regulatory functions. Their cells expressed the progenitor marker Cd34 (FIG. 2G) and MSC markers (Ly6a, Pdgfra, Thy1 and Cd44; FIGS. 8D-8E), but not EC or pericytes genes (Cdh5, Acta2 FIG. 1C), suggesting these fibroblasts have MSC characteristics. Fibroblast-1 also express the hematopoietic regulatory cytokines Cxcl12 and Angpt1 (FIGS. 3B and 7G) indicating a possible niche regulatory function that resembles CAFs that facilitate cancer growth, metastasis (Ahirwar et al., 2018), and immunosuppression through Cxcl12 (Costa et al., 2018).

Fibroblasts-3, 4, and 5s were related to the tendon/ligamentous junction, from tenocyte progenitors to tendon/ligament cells. Fibroblast-3 (cluster 16), Fibroblast-4 (Cluster 3) and Fibroblast-5 (cluster 5) co-expressed Sox9 (FIG. 7B) and the TF Scleraxis (Scx) (FIG. 8F) that regulate differentiation in tenocytes and ligamentocytes in tendons and ligaments (Sugimoto et al., 2013). Fibroblast-4 and -5 further expressed key bone and cartilage genes, including the osteogenic Spp1 (FIG. 6B) and the chondrocyte genes Cspg4, CD73 (Nt5e) (FIG. 4A) and Cartilage Intermediate Layer Protein (Cilp) (FIG. 8F). The three clusters formed a continuum from Fibroblasts-3 to Fibroblast-4 and -5 (FIG. 7F). Fibroblast-3s may be tenocyte progenitors (Sugimoto et al., 2013), whereas Fibroblast-4 and -5s consists of tendon/ligament cells that are located close to the chondro-tendinous/ligamentous junction (Sugimoto et al., 2013).

### Example 7—EC Progenitor Express Higher Levels of Niche Factors Compared to Sinusoidal and Arteriolar Vascular ECs

Bone marrow derived endothelial cells (BMECs) are a heterogeneous cell population (Breitbach et al., 2018) from either arteriolar or sinusoidal blood vessels, where they act as critical regulators of HSC function through secretion of niche factors (Butler et al., 2010; Ding et al., 2012; Doan et al., 2013; Himburg et al., 2012; Hooper et al., 2009; Kiel et al., 2005; Kobayashi et al., 2010; Muramoto et al., 2006).

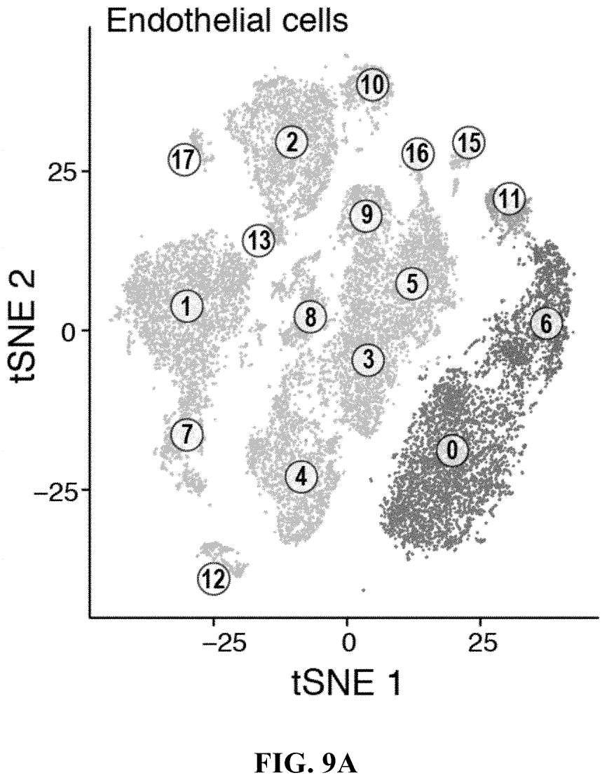

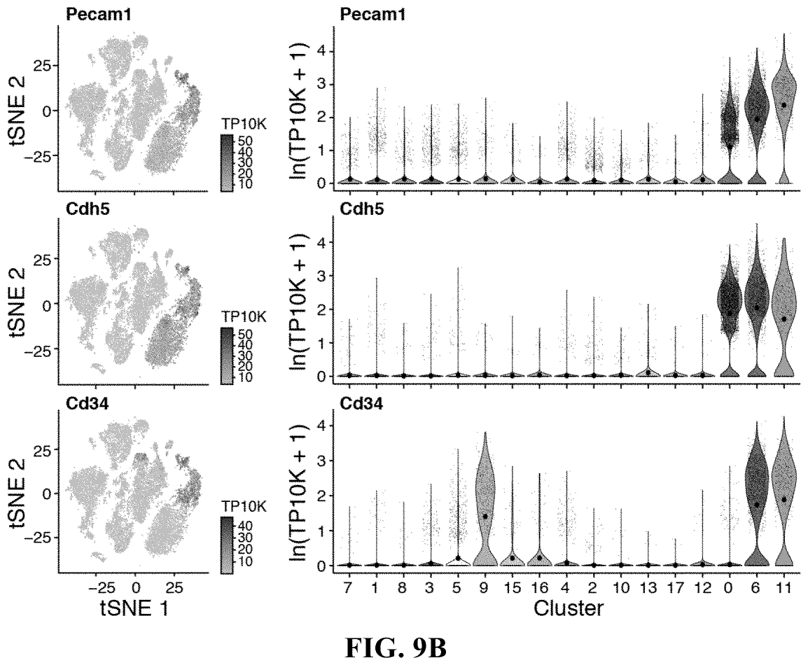

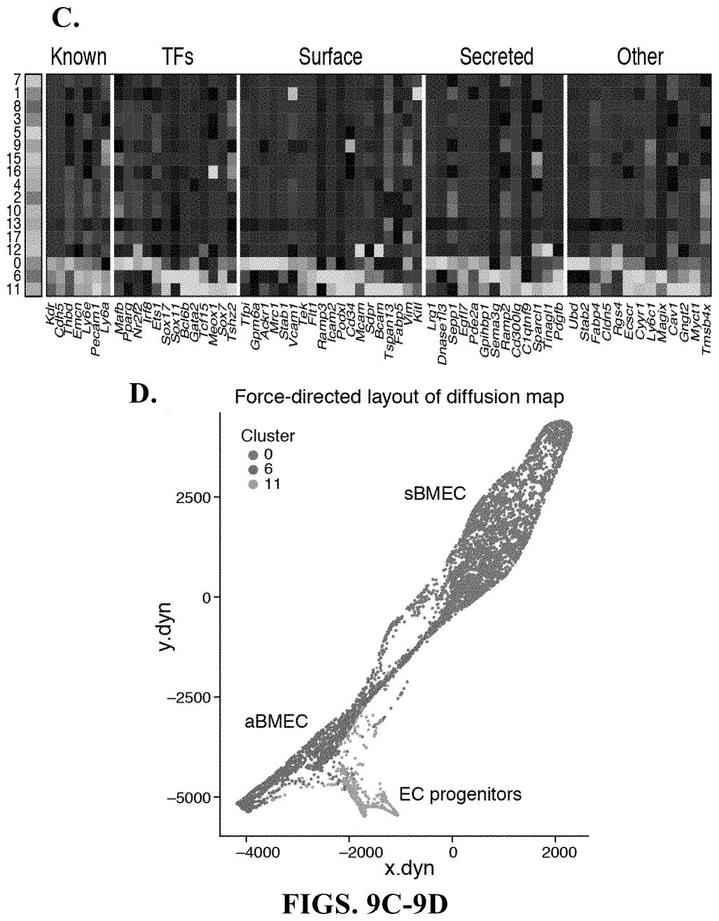

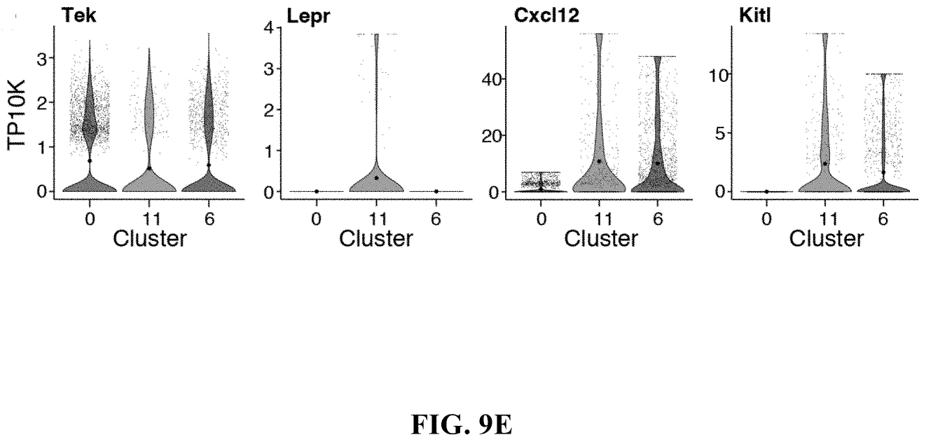

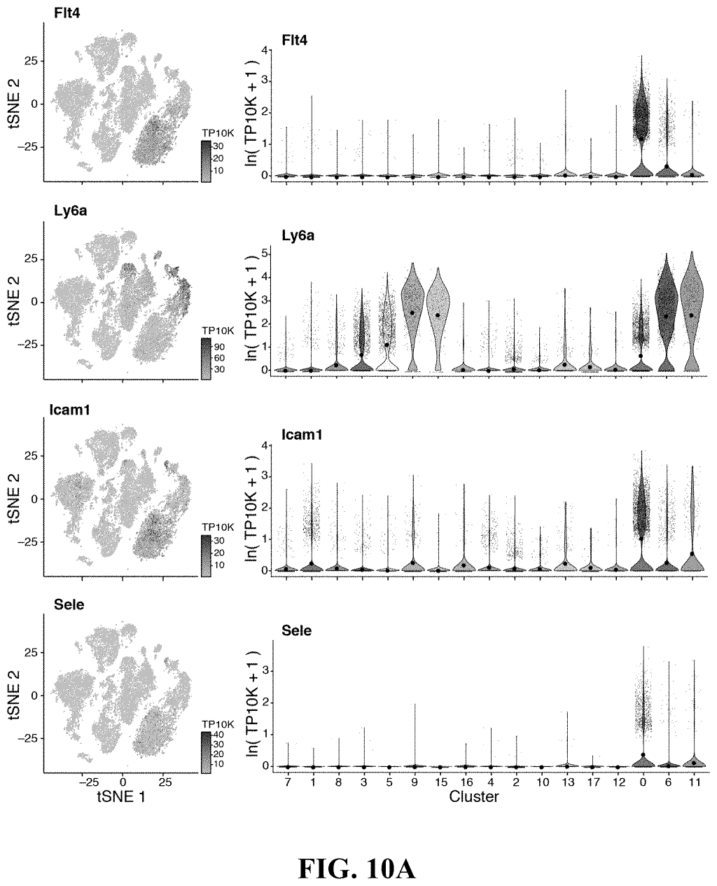

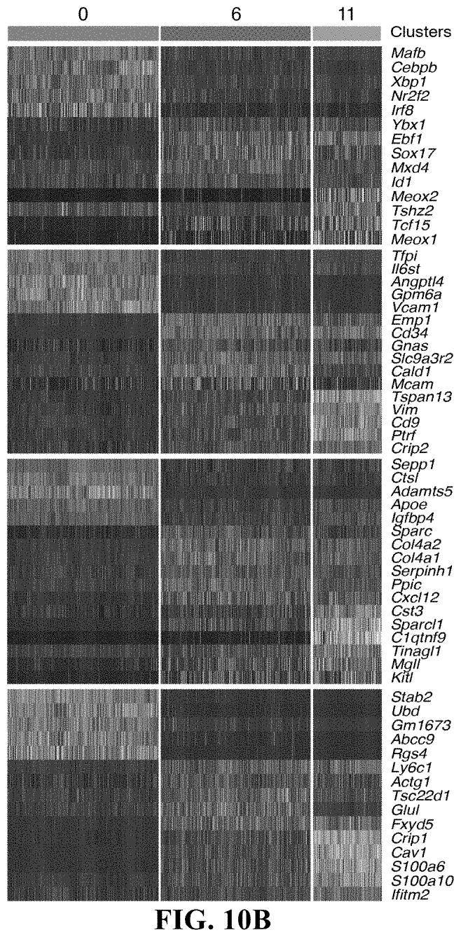

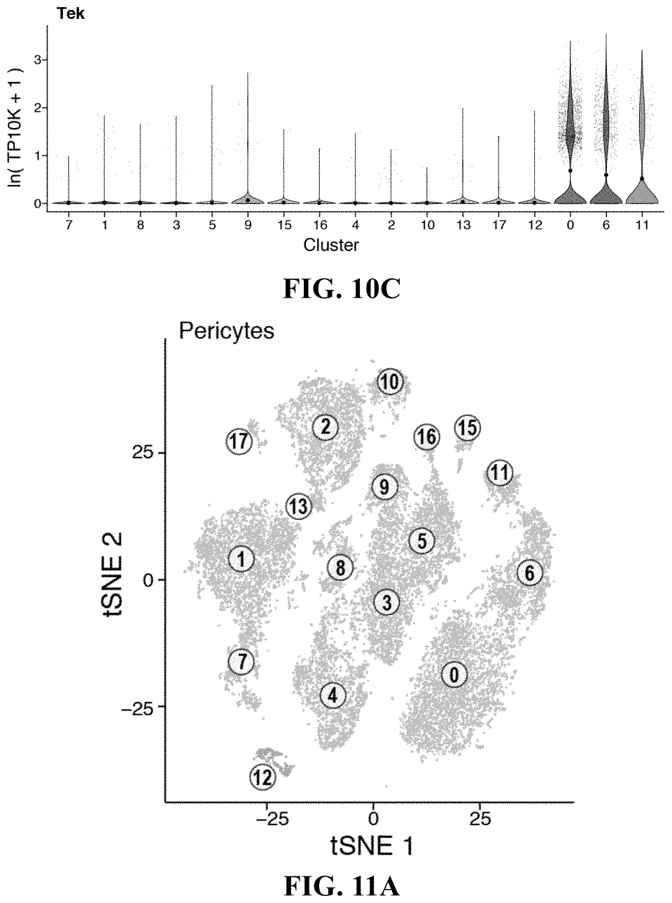

Three BMEC subsets were identified (Clusters 11, 6, and 0)—annotated as EC progenitors, arterial BMECs, and sinusoidal BMECs, respectively. All subsets expressed Pecam1, Cdh5, Kdr, and Emcn (Rafii et al., 2016) (FIGS. 9A-9C), and related along a continuum (FIG. 9D) In particular, Cd34, commonly expressed in primitive endothelial and hematopoietic cells (Asahara et al., 1997; Yoder, 2012), was highly expressed in both cluster 6 and cluster 11, indicating an immature cell state. Diffusion map analysis (Angerer et al., 2016) (FIG. 9D) suggests that cluster 11 cells are EC progenitors that differentiate into cluster 6 cells. We annotated cluster 6 and 0 cells as arterial and sinusoidal BMECs (aBMECs and sBMECs), respectively, by marker expression: Cluster 0 cells had high expression of Flt4 (Vegfr-3) and low expression of Ly6a (Sca-1), and cluster 6 cells had the opposite pattern (FIG. 10A). This was consistent with previous reports that sinusoidal BMECs are Vegfr-3+/Sca-1⁻, while arterioles that branch from arterial BMECs are Vegfr-3-/Sca-1⁺ (Hooper et al., 2009; Itkin et al., 2016).

The three subsets expressed different ligands and secreted factors. All three expressed the endothelial tyrosine kinase receptor, Tie2 (Tek gene product) for angiopoietin ligands. However, the hematopoietic factors, Kitl and Cxcl12 were most abundant in the EC progenitors, aBMECs expressed Cxcl12 with minimal Kit1, and sBMECs did not express either factor (FIGS. 9E and 10C). Thus, EC progenitors may be a distinctively relevant subpopulation within ECs, that regulates hematopoietic stem and progenitor cell function (Ding et al., 2012; Xu et al., 2018).

### Example 8—NG2⁺ and Nestin⁺ Pericytes are Distinct from LepR⁺ MSCs

NG2⁺ and Nestin⁺pericytes have been proposed as critical regulators of HSC function through production of Cxcl2 and Kitl (Kunisaki et al., 2013; Mendez-Ferrer et al., 2010). Notably, under homeostasis, HSCs reside in perisinusoidal or periarteriolar locations predominantly found in close proximity to LepR-cre positive cells, also shown to produce Cxcl2 and Kitl (Acar et al., 2015; Morrison and Scadden, 2014). However, determining whether NG2⁺ and Nestin⁺ pericytes share developmental origins and functional properties with perivascular LepR⁺ MSCs (Armulik et al., 2011) has been challenging as there is currently no single marker that identifies them without overlapping with MSCs (Armulik et al., 2011).

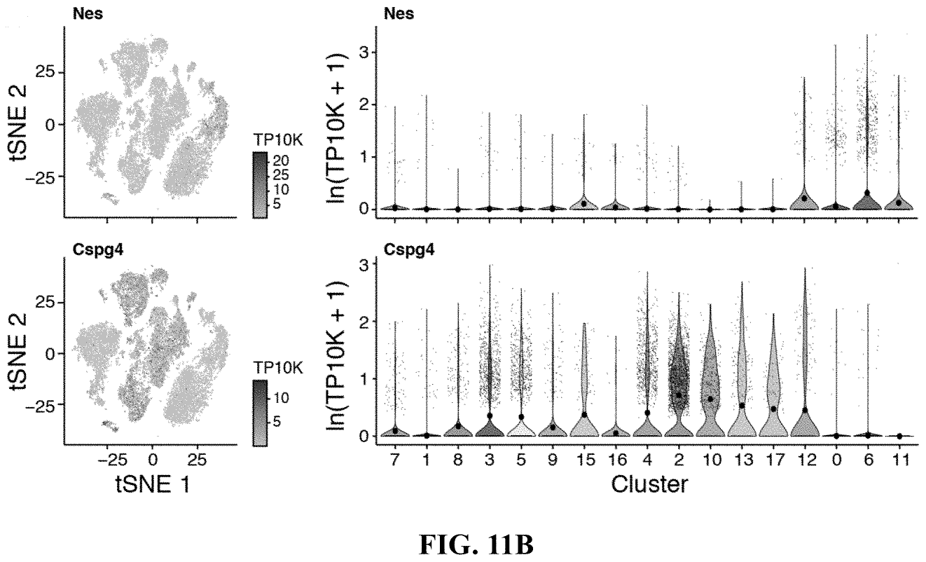

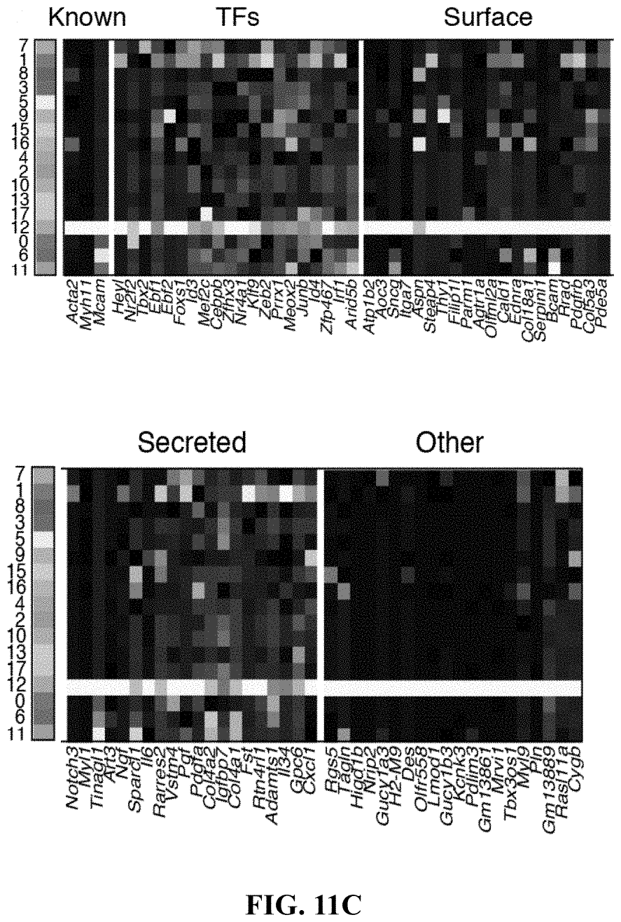

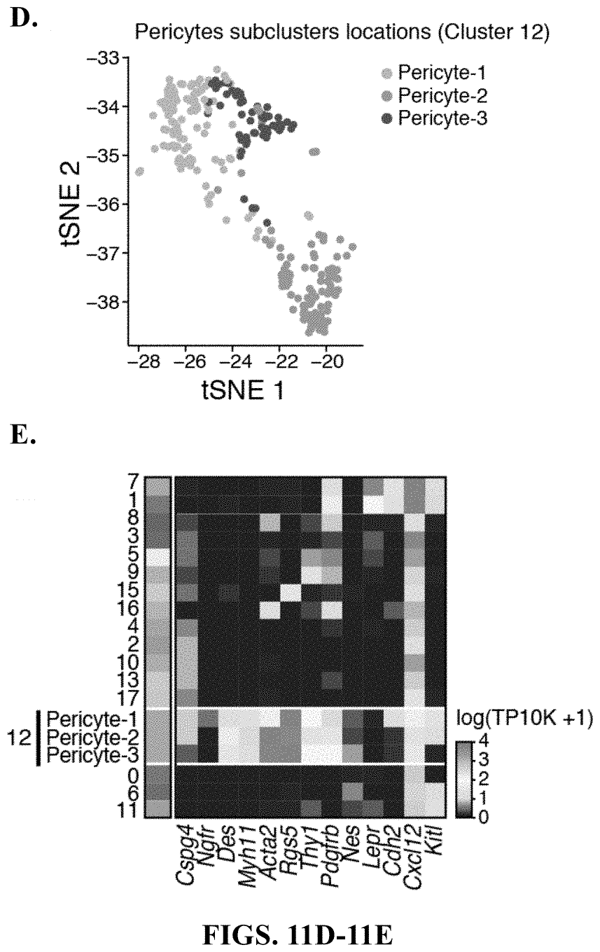

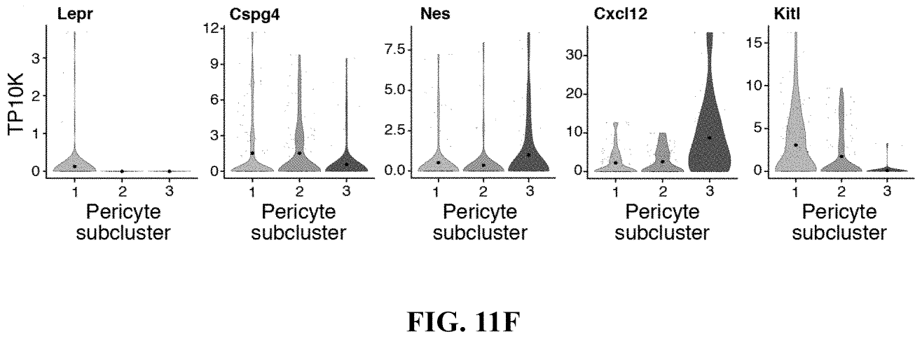

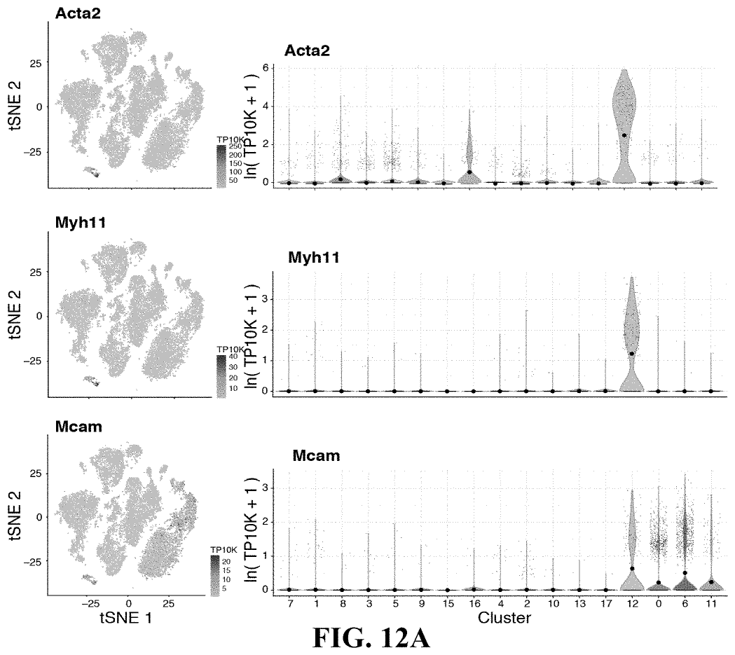

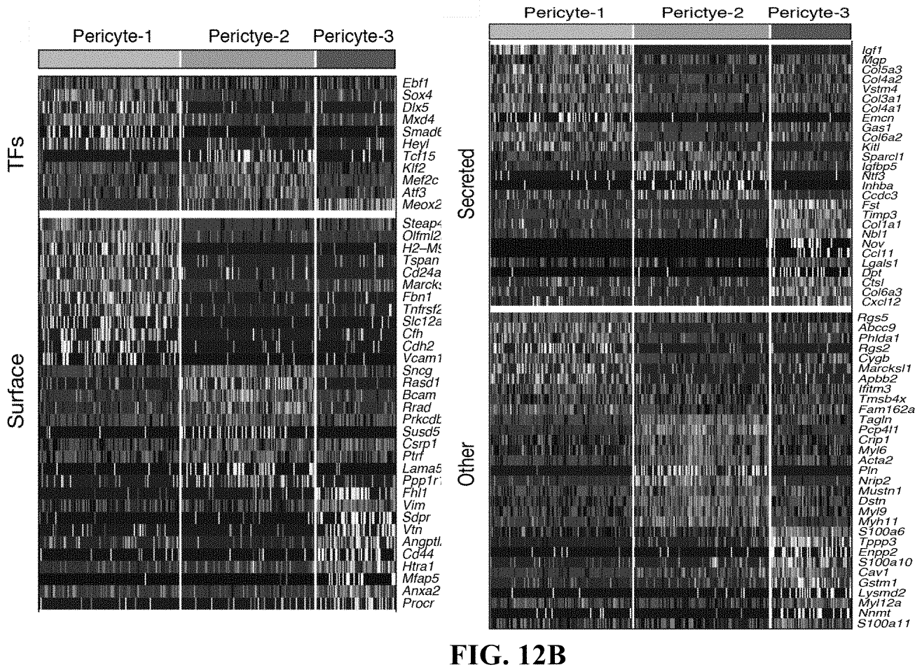

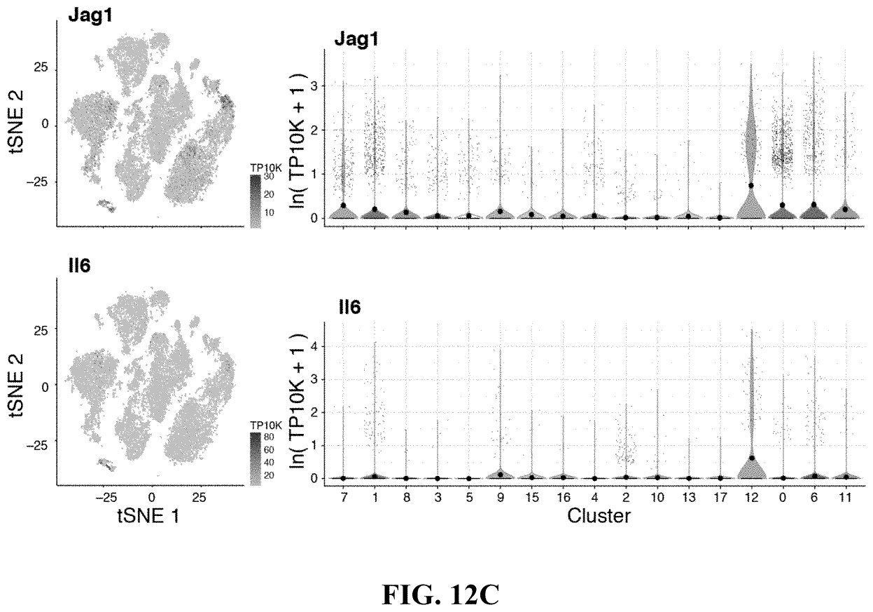

A distinct cluster of pericytes (Cluster 12) was annotated, by the co-expression of the classical markers Nestin (Nes), NG2 (Cspg4), α-smooth muscle actin (Acta2), myosin (Myh11) and Mcam (Armulik et al., 2011) (FIGS. 11A-11C and 12A). The pericytes differentially express Jag1, the Notch signaling ligand, and 11-6, both implicated in promoting HSC maintenance (Bernad et al., 1994; Calvi et al., 2003) (FIG. 12C). Notably, cells in this pericyte cluster express only very low levels of Lepr, in contrast to the high expression of Lepr in MSCs (FIGS. 3B-3C). Thus, perivascular MSCs and pericytes are clearly distinguishable.

Within the pericytes, we identified three subsets (FIGS. 11D and 12B). Pericyte-1 cells, distinguished by relatively higher expression of Cdh2 and specific expression of MSC surface marker Ngfr (CD271) (FIG. 11E), expressed Lepr, Cxcl12, Cspg4 and Nes (albeit at low levels for the four genes) and Kit1, and may reflect a subset of LepR⁺ cells in the pericyte pool that contribute to HSC regulation (FIGS. 11-11F). Conversely, LepR− pericytes express either Cspg4 (NG2, Pericyte-2s) or Nes (Pericyte-3s) which have previously characterized, respectively, periarteriolar cells (NG2-CreER/Nestin-GFP^{high}, (Coutu et al., 2017; Kunisaki et al., 2013) and perisinusoidal cells (Nestin-GFP⁺, (Mendez-Ferrer et al., 2010), co-localizing with HSC and serving as HSC niche cells. In marked contrast to MSCs, none of these subsets expresses high levels of both Cxcl12 andKitl (FIGS. 3F and 11E-11F). Therefore, while LepR⁺ cells, Cspg4⁺ (NG2) or Nes⁺ cells have been defined as HSC niche cells, they are molecularly distinct. Cspg4 (NG2) and Nes expressing pericytes are not among the broad range of LepR⁺ cells (Ding and Morrison, 2013; Ding et al., 2012), and their distinct expression of Cxcl2 and Kitl suggests they may have distinct niche functions.

### Example 8—Systemic Survey of Bone Marrow Stromal Cell Changes Induced by Primary MLL-AF9 Leukemia

Several bone marrow stromal cell populations that regulate hematopoiesis, can, when perturbed, lead to niche-initiated myelodysplasia or leukemia (Arranz et al., 2014; Dong et al., 2016; Kode et al., 2014; Raaijmakers et al., 2010). Moreover, myeloid and lymphoblastic leukemias can remodel their niche to support malignant growth (Hanoun et al., 2014; Schepers et al., 2013; Schmidt et al., 2011).

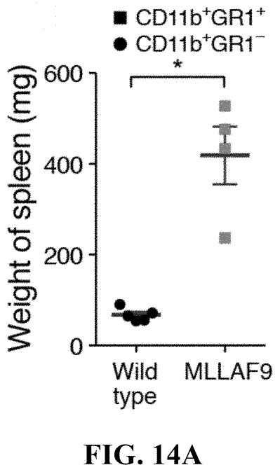

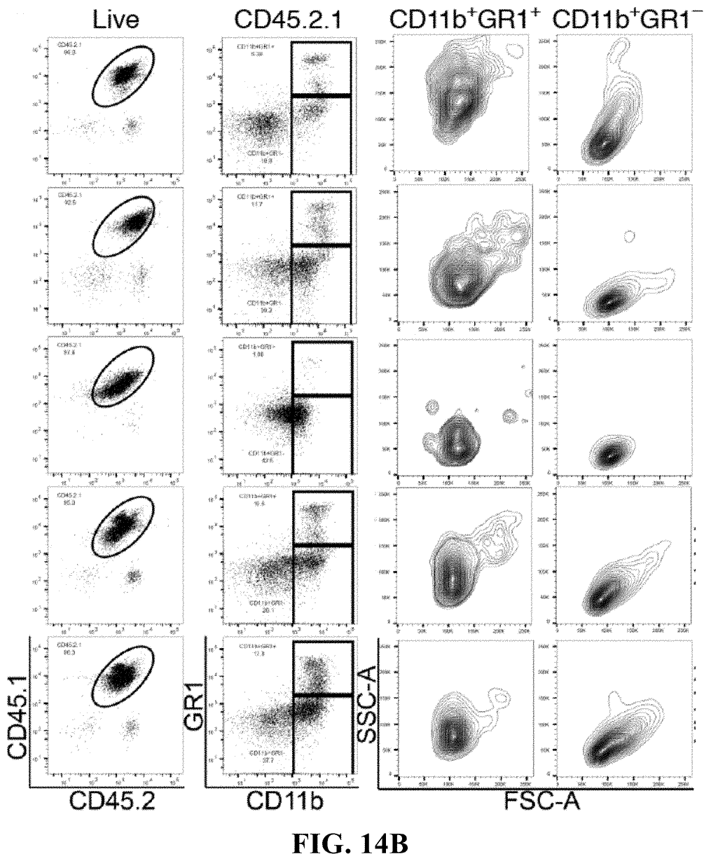

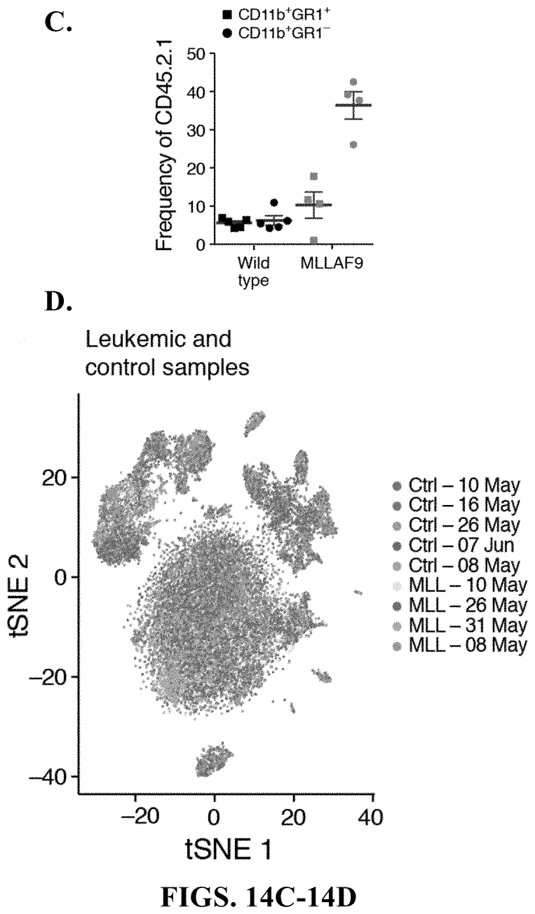

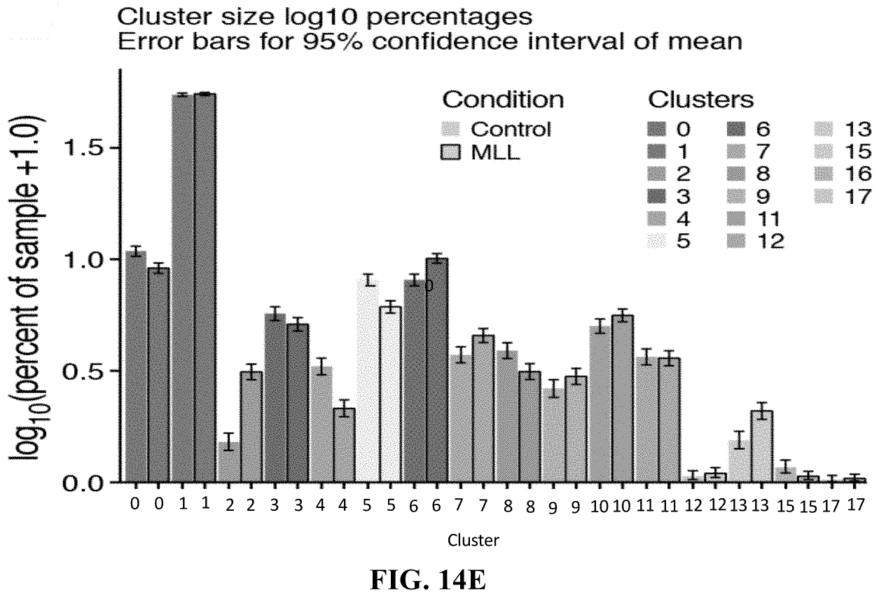

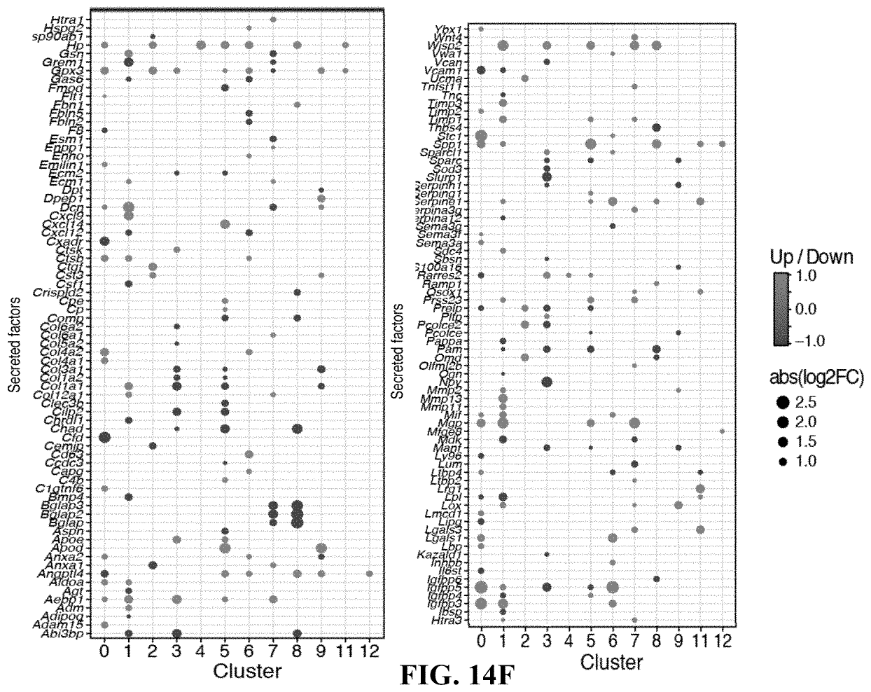

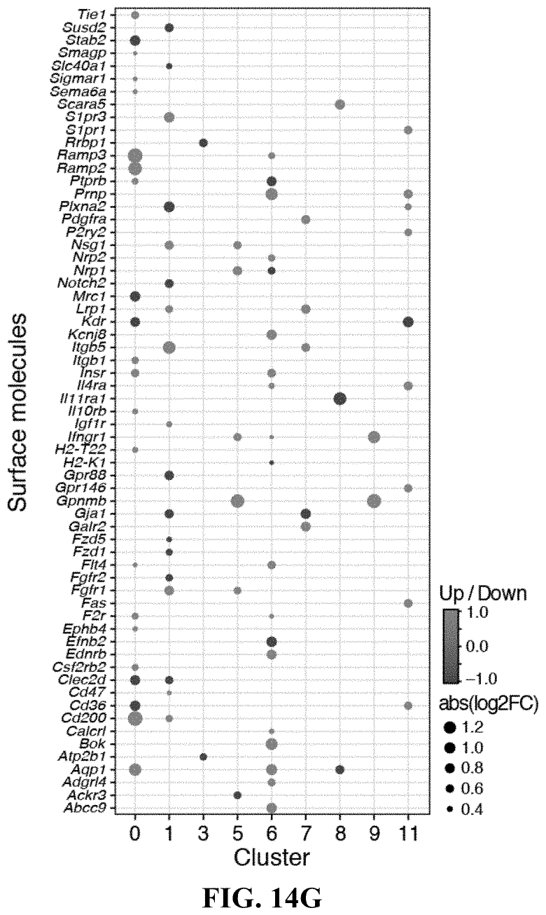

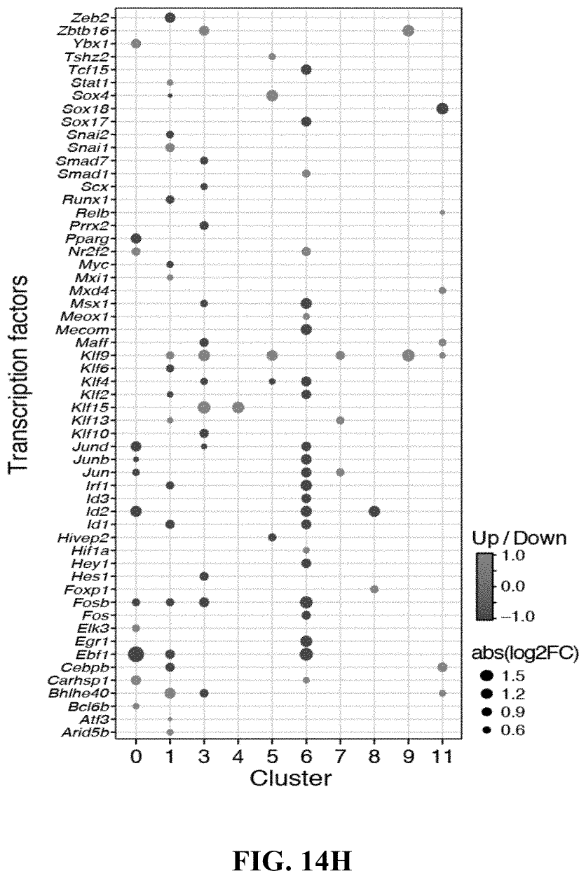

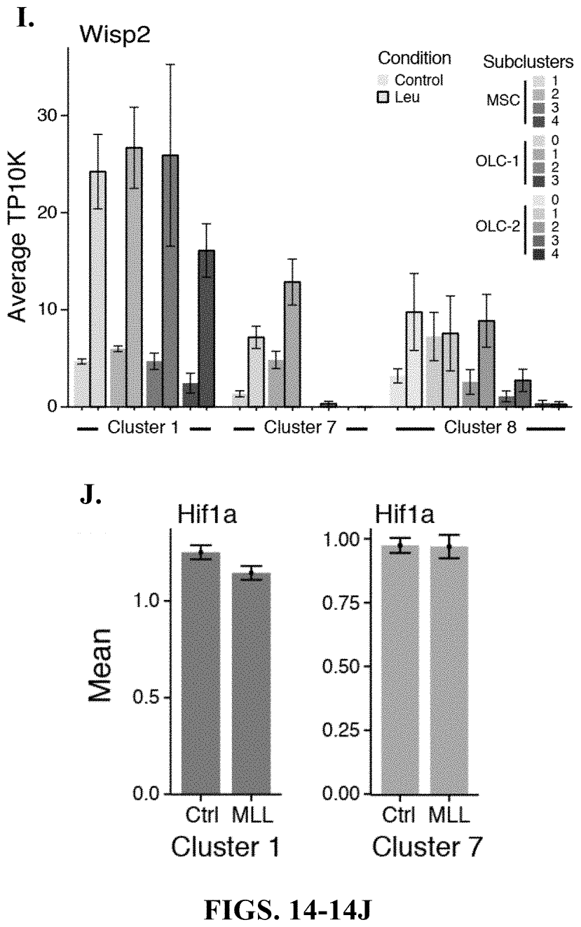

To comprehensively assess changes in the stroma during malignant growth, a mouse model (Corral et al., 1996) was used, where primary MLL-AF9 knock-in bone marrow donor cells (4-6 week old mice) were transplanted into lethally irradiated congenic wild-type recipient mice to generate leukemic mice, and compared them to bone marrow transplants from mice without MLL-AF9. More than 6 months of normal hematopoiesis elapsed in both populations before leukemia was detected in the MLL-AF9 bearing mice. Mice showing signs of emerging leukemia, enlarged spleens (FIG. 14A) and leukemic blasts by FACS scatter and Cd11b⁺/Grl⁻ expression within the myeloid compartment in peripheral blood (FIGS. 14B-14C) were sacrificed about 6-10 months post-transplantation.

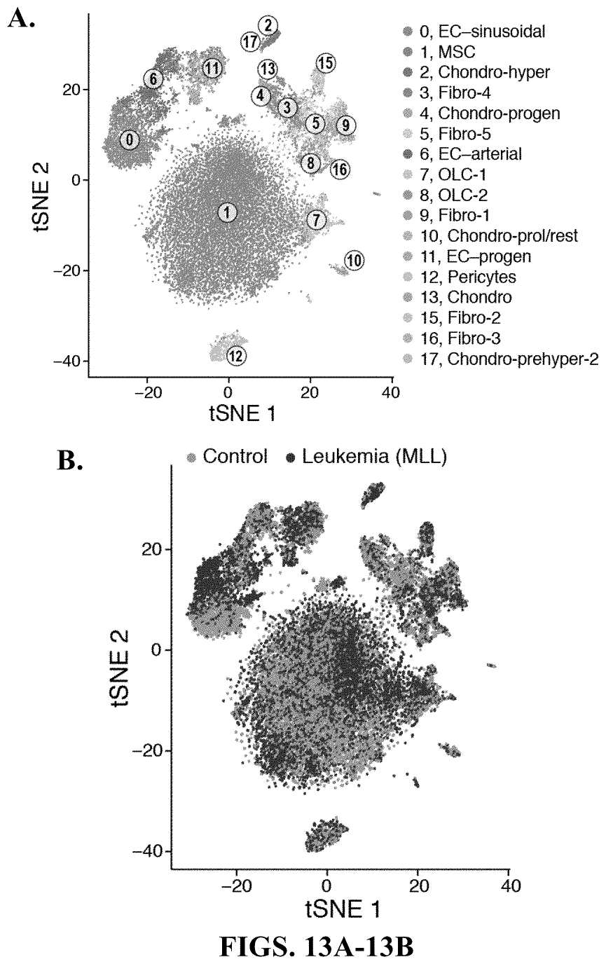

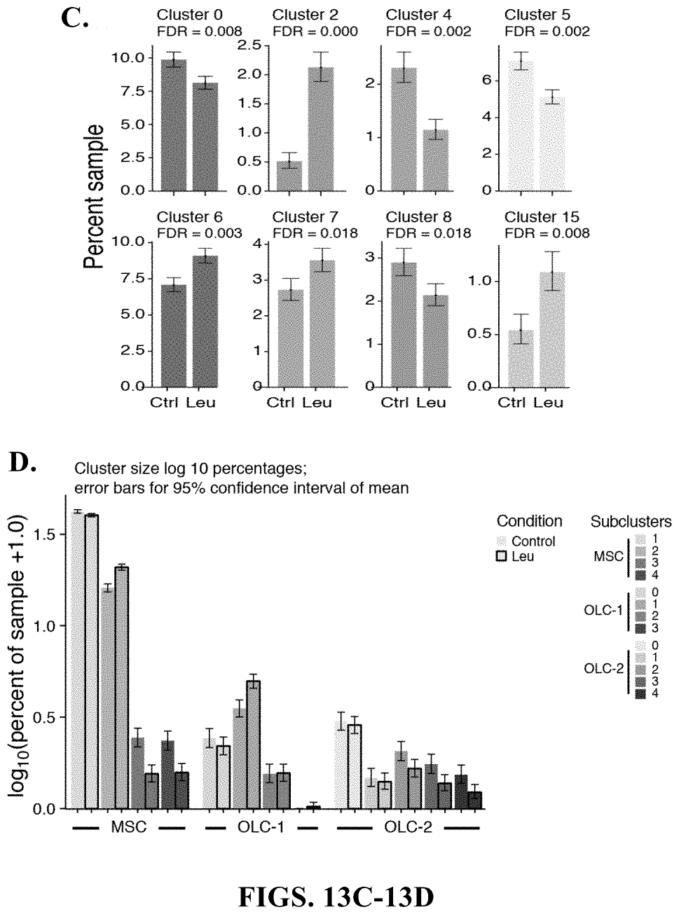

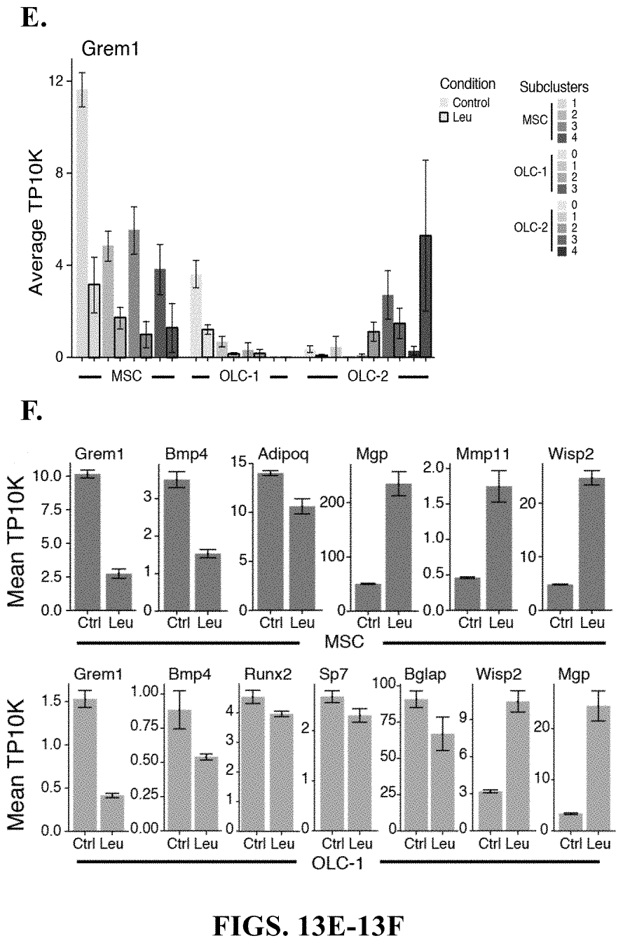

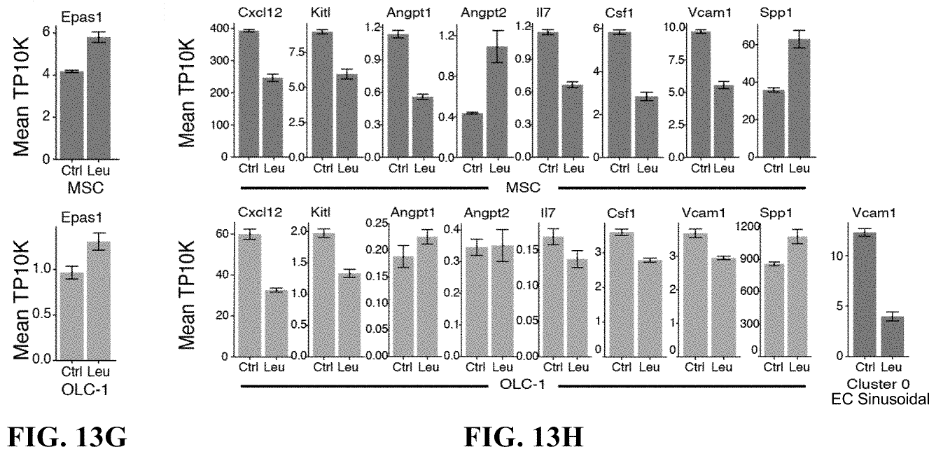

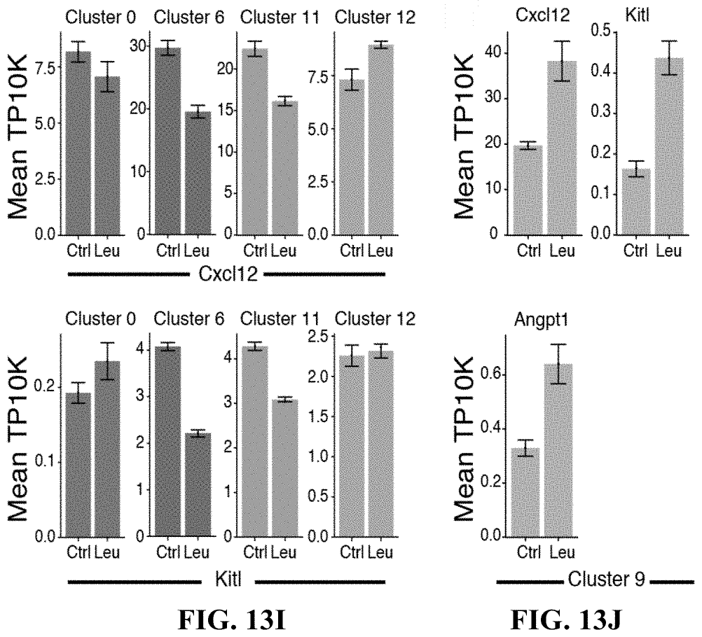

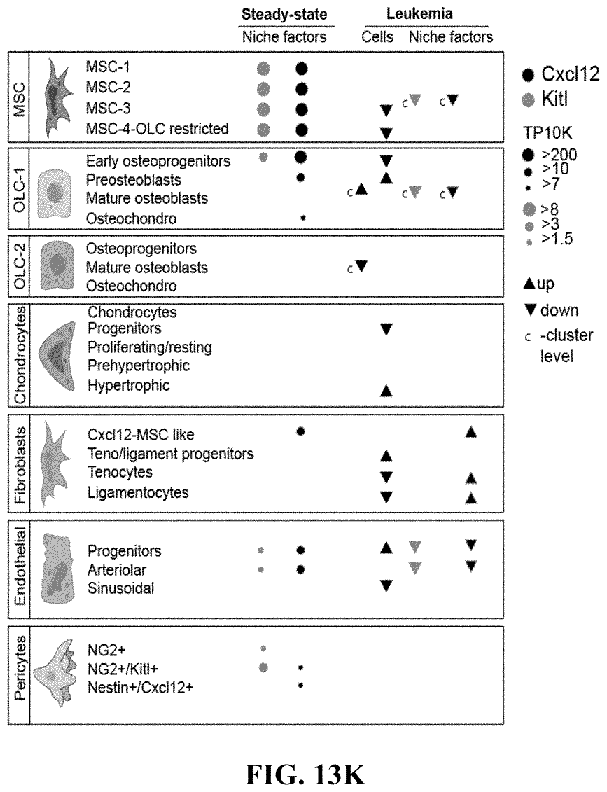

scRNA-seq was used to profile 12,456 bone marrow stromal cells from MLL-AF9 (MLL) mice (n=4) and 10,548 cells from matched control transplanted mice (n=5). Cells were clustered from all mice together, and assigned cell-type identity based on gene signatures from our steady-state analysis (FIGS. 13A-13B and 14D). Then for each cluster/cell-type we tested for statistically significant differences in the proportions of the subsets.

### Example 9—Remodeling of OLCs, ECs, Chondrocytes, and Fibroblasts Proportions in Leukemia

Significant changes were detected in the proportions of key subsets in leukemia. These often reflected coupled effects in OLCs (increase in OLC-1, reduction in OLC-2), ECs (reduction in sBMECs, increase in aBMECs), chondrocytes (increase in cluster 2, decrease in 4) and fibroblasts (decrease in Fibroblast-3, increase in Fibroblast-4) (FIGS. 13C and 14E).

The shift in OLCs, including reduction in more mature bone cells, while maintaining Nes⁺ pericytes, is consistent with previous studies of leukemia, where OLC lineage dysfunction and loss of mature cells is caused by leukemia cells and favorable for their growth (Duarte et al., 2018; Frisch et al., 2012; Hawkins et al., 2016; Krevvata et al., 2014). Moreover, proportions also varied within the OLC-1 subset, with a significant increase in preosteoblasts (sub-cluster 7-1) but not in other cells (FIG. 13D). While the overall proportion of MSCs was unchanged (FIG. 14E), the relative proportions within MSCs were impacted by leukemia, with significant increase in MSC-2 and a concomitant reduction in MSC-3 and MSC-4 (committed osteolineage MSCs) (FIG. 13D).

Overall, the increase in osteoprogenitors (e.g., preosteoblasts) accompanied with a reduction of committed osteolineage MSCs suggests that leukemia induced a block in osteolineage development (Kumar et al., 2018). The changes observed in OLCs and MSCs, as well as ECs, are consistent with the reported abnormalities seen in AML patients, where leukemia induces vasculature remodeling that is accompanied by reduced osteocalcin (Bglap) serum levels, growth deficiency, and impaired osteogenesis (Duarte et al., 2018; Geyh et al., 2016; Kumar et al., 2018; Passaro et al., 2017).

### Example 10—Leukemia Compromises Osteogenic and Adipogenic Differentiation Pathways in MSCs and OLCs

Analyzing changes in intrinsic gene programs within each cluster (Table 7, FIGS. 14F-141) highlighted a blunted adipogenic and osteogenic differentiation programs in MSCs and OLCs. Cells in both MSC and OLC clusters downregulated the skeletal stem cell marker Grem1 (Worthley et al., 2015), across all MSC sub-clusters and in OLC progenitors (FIG. 13E), as well as the BMP pathway, including Bmp4 (GSEA qval=0.027, Table 8) (Subramanian et al., 2005) (GSEA qval=0.027) (Wozney et al., 1988), and the early osteolineage cell-specification gene Sp7 (FIG. 13F). Mature osteoblasts within OLCs further downregulated Runx2, an early marker of osteogenesis, and Bglap1, Bglap2 and Bglap3, late markers of osteogenesis (Lee et al., 2007) (FIGS. 13F and 14F). MSC cells further downregulated Adipoq (Bauche et al., 2007) and other genes in the white adipocyte differentiation (GSEA qval=0.029, Table 8) and the PPAR signaling pathways (GSEA qval=0.022, Table 8) (FIG. 13F, FIG. 14F) (Tontonoz et al., 1994). This is consistent with reports that a compromised adipocytic niche favors leukemia growth (Boyd et al., 2017). Furthermore, Wisp2, which has been implicated in restricting MSCs to an undifferentiated state (Grunberg et al., 2014; Hammarstedt et al., 2013) was upregulated in all MSC subsets and OLC progenitors (within OLCs) (FIGS. 14F and 14I), further supporting compromised differentiation of MSCs and OLCs. Conversely, key genes that inhibit bone formation and calcification were induced in MSCs and OLCs (and others), including Mgp (FIG. 13F) (inhibits calcification, (Luo et al., 1997)); Igbfp5 and Igfbp3 (negative regulators on bone formation (Kanatani et al., 2000; Silha et al., 2003), FIG. 14G), and genes in the ECM degradation pathway (GSEA qval=0.04, Table 8, FIG. 13E), including Mmp2, Mmpl1 and Mmp13 (FIG. 14G). Taken together, these data are consistent with AML inducing alterations that affect bone formation and breakdown.

The cell profiles further support a model (Kim et al., 2009) where hypoxia accounts for the undifferentiated state of the MSCs and OLCs. Hypoxia pathway genes (GSEA qval=0.004, Table 8) were induced in MSCs and OLCs, including the key regulator Hif-2a (Epasl) (FIGS. 13G and 1411), but not Hif-1a (FIG. 14J), consistent with reports that suppression of bone formation and osteoblast activity is directly linked to Hif-2a and not Hif-1a (Rauner et al., 2016). Notably, the Wisp2 promoter is regulated by Hif-2a (Fuady et al., 2014).

TABLE 7

| Collect | Cluster | Enrich | Set | pval | qval | Genes |
|---|---|---|---|---|---|---|
| c1 | 1 | down | CHR8P22 | 0.000 | 0.033 | Azin1, Ccar2, Lpl, Trib1 |
| c1 | 1 | down | CHR8Q23 | 0.000 | 0.043 | Csmd3, Mal2, Rspo2, Slc26a7, |
| | | | | | | Sybu |
| c1 | 1 | up | CHR3P26 | 0.000 | 0.022 | Bhlhe40, Lmcd1 |
| c1 | 1 | up | CHR8P23 | 0.000 | 0.013 | Angpt2, Csmd1, Ctsb, Kbtbd11 |
| c1 | 3 | up | CHR16Q13 | 0.000 | 0.019 | Mmp2, Mt1 |
| c1 | 3 | up | CHR6Q23 | 0.000 | 0.004 | Ctgf, Ifngr1, Sgk1 |
| c1 | 6 | down | CHR6P21 | 0.000 | 0.000 | Brd2, Cdkn1a, Egfl8, Hfe, Hspa1a, |
| | | | | | | Hspa1b, Ier3, Neu1, Ppp1r10, |
| | | | | | | Ppt2, Psmb8, Psmb9, Rpp21, |
| | | | | | | Scube3, Tap2, Tapbp |
| c2 | 0 | down | BIOCARTA EPONFKB | 0.007 | 0.046 | Grin1, Hif1a, Jak2, Nfkbia |
| | | | PATHWAY | | | |
| c2 | 0 | down | BIOCARTA IL12 PATHWAY | 0.007 | 0.043 | Jak2, Jun, Map2k6 |
| c2 | 0 | down | BIOCARTA IL4 PATHWAY | 0.000 | 0.048 | Jak1, Jak3 |
| c2 | 0 | down | PID IL27 PATHWAY | 0.000 | 0.035 | Il6st, Jak1, Jak2, Stat5a |
| c2 | 0 | down | REACTOME IL 6 SIGNALING | 0.000 | 0.001 | Il6st, Jak1, Jak2, Tyk2 |
| c2 | 0 | down | REACTOME INITIAL | 0.007 | 0.029 | Cfd, Crp |
| | | | TRIGGERING OF | | | |
| | | | COMPLEMENT | | | |
| c2 | 0 | down | ST IL 13 PATHWAY | 0.000 | 0.025 | Il13ra2, Jak1, Jak2 |
| c2 | 0 | up | BIOCARTA ACE2 PATHWAY | 0.000 | 0.035 | Col4a1, Col4a2 |
| c2 | 0 | up | BIOCARTA PLATELETAPP | 0.000 | 0.019 | App, Col4a1, Col4a2 |
| | | | PATHWAY | | | |
| c2 | 0 | up | BIOCARTA VITCB PATHWAY | 0.000 | 0.043 | Col4a1, Col4a2 |
| c2 | 0 | up | REACTOME APOPTOTIC | 0.000 | 0.028 | Lmna, Plec, Vim |
| | | | CLEAVAGE OF CELLULAR | | | |
| | | | PROTEINS | | | |
| c2 | 0 | up | REACTOME APOPTOTIC | 0.000 | 0.004 | H1f0, Lmna, Plec, Vim |
| | | | EXECUTION PHASE | | | |
| c2 | 0 | up | REACTOME REGULATION OF | 0.000 | 0.013 | Igfbp3, Igfbp5 |
| | | | INSULIN LIKE GROWTH | | | |
| | | | FACTOR IGF ACTIVITY BY | | | |
| | | | INSULIN LIKE GROWTH | | | |
| | | | FACTOR BINDING PROTEINS | | | |
| | | | IGFBPS | | | |
| c2 | 0 | up | REACTOME SHC RELATED | 0.000 | 0.037 | Insr, Kras, Map2k1, Mapk3 |
| | | | EVENTS | | | |
| c2 | 0 | up | REACTOME THROMBIN | 0.003 | 0.041 | F2r, Gna11, Gnb2, Gng13, Gngt2, |
| | | | SIGNALLING THROUGH | | | Mapk3 |
| | | | PROTEINASE ACTIVATED | | | |
| | | | RECEPTORS PARS | | | |
| c2 | 13 | up | REACTOME OLFACTORY | 0.000 | 0.000 | Gnb1 |
| | | | SIGNALING PATHWAY | | | |
| c2 | 15 | down | PID P53 DOWNSTREAM | 0.000 | 0.011 | Atf3, Bak1, Btg2, Crebbp, Kat2a, |
| | | | PATHWAY | | | Map4k4, Mdm2, Mmp2, Rrm2b |
| c2 | 15 | up | BIOCARTA ECM PATHWAY | 0.001 | 0.001 | Fyn, Gsn, Hras, Map2k1 |
| c2 | 15 | up | PID INTEGRIN A9B1 | 0.001 | 0.044 | F13a1, Fn1, Tgm2 |
| | | | PATHWAY | | | |
| c2 | 16 | down | REACTOME OLFACTORY | 0.000 | 0.032 | Gnb1, Olfr519 |
| | | | SIGNALING PATHWAY | | | |
| c2 | 16 | down | REACTOME PEPTIDE LIGAND | 0.000 | 0.000 | App, Cel19, Cxcl1, Edn3 |
| | | | BINDING RECEPTORS | | | |
| c2 | 1 | down | BIOCARTA AKT PATHWAY | 0.000 | 0.033 | Foxo3, Ghr, Nfkbia, Pik3r1 |
| c2 | 1 | down | BIOCARTA CARDIACEGF | 0.004 | 0.047 | Agt, Myc, Plcg1 |
| | | | PATHWAY | | | |
| c2 | 1 | down | BIOCARTA INFLAM PATHWAY | 0.000 | 0.023 | Csf1, Il15, Il7, Pdgfa |
| c2 | 1 | down | BIOCARTA VIP PATHWAY | 0.000 | 0.041 | Myc, Nfkbia, Plcg1 |
| c2 | 1 | down | KEGG HEMATOPOIETIC CELL | 0.000 | 0.034 | Cd55b, Csf1, Il7, Kitl |
| | | | LINEAGE | | | |
| c2 | 1 | down | KEGG NOD LIKE RECEPTOR | 0.002 | 0.040 | Ccl2, Ccl7, Cxcl1, Nfkbia, Nfkbib |
| | | | SIGNALING PATHWAY | | | |
| c2 | 1 | down | KEGG PPAR SIGNALING | 0.000 | 0.022 | Acox1, Adipoq, Angptl4, Gk2, Lpl |
| | | | PATHWAY | | | |
| c2 | 1 | down | PID BMP PATHWAY | 0.000 | 0.027 | Bambi, Bmp4, Chrdl1, Fst, Grem1, |
| | | | | | | Ppp1r15a |
| c2 | 1 | down | PID IL3 PATHWAY | 0.000 | 0.031 | Cebpb, Csf2rb2, Id1, Pik3r1, Pim1, |
| | | | | | | Prkaca, Ywhag |
| c2 | 1 | down | PID INTEGRIN A4B1 | 0.000 | 0.036 | Cd81, Crk, Mdk, Prkaca, Vcam1 |
| | | | PATHWAY | | | |
| c2 | 1 | down | REACTOME | 0.000 | 0.029 | Adipoq, Angptl4, Cebpb, Ebf1, |
| | | | TRANSCRIPTIONAL | | | Lpl, Med22, Tgs1 |
| | | | REGULATION OF WHITE | | | |
| | | | ADIPOCYTE | | | |
| | | | DIFFERENTIATION | | | |
| c2 | 1 | up | PID HIF2PATHWAY | 0.000 | 0.010 | Bhlhe40, Epas1, Serpine1, Sirt1, |
| | | | | | | Sp1, Vegfa |
| c2 | 1 | up | PID TAP63 PATHWAY | 0.000 | 0.000 | Cdkn1a, Gadd45a, Igfbp3, Mfge8, |
| | | | | | | Sp1, Vdr |
| c2 | 1 | up | PID UPA UPAR PATHWAY | 0.000 | 0.013 | Itgb5, Lrp1, Serpine1 |
| c2 | 1 | up | REACTOME DEGRADATION | 0.000 | 0.000 | Mmp11, Timp1, Timp2 |
| | | | OF THE EXTRACELLULAR | | | |
| | | | MATRIX | | | |
| c2 | 1 | up | REACTOME GLYCOLYSIS | 0.002 | 0.016 | Eno1, Pfkl, Pgam1, Tpi1 |
| c2 | 3 | down | REACTOME ACTIVATION OF | 0.000 | 0.003 | Atf4, Calr, Hsp90b1, Hspa5, Xbp1 |
| | | | CHAPERONES BY ATF6 ALPHA | | | |
| c2 | 3 | down | REACTOME AXON GUIDANCE | 0.000 | 0.009 | Cacnb2, Col1a1, Col1a2, Col3a1, |
| | | | | | | Col5a2, Col6a1, Col6a2, Col6a3, |
| | | | | | | Dpysl2, Rhoa, Rhob, Siah2 |
| c2 | 3 | down | REACTOME COLLAGEN | 0.000 | 0.013 | Adamts2, Col15a1, Col1a1, |
| | | | FORMATION | | | Col1a2, Col3a1, Col5a2, Col6a1, |
| | | | | | | Col6a2, Col6a3, Col8a2, Colgalt1, |
| | | | | | | P3h2, Pcolce, Pcolce2, Plod2, |
| | | | | | | Serpinh1 |
| c2 | 5 | down | REACTOME COLLAGEN | 0.000 | 0.024 | Col1a1, Col1a2, Col3a1, Col5a1, |
| | | | FORMATION | | | Col6a1, Col6a2, Col8a1, Col8a2, |
| | | | | | | Pcolce, Plod2, Serpinh1 |
| c2 | 5 | down | REACTOME SIGNALING BY | 0.000 | 0.002 | Adcy4, Calm1, Calm2, Col1a1, |
| | | | PDGF | | | Col1a2, Col3a1, Col5a1, Col6a1, |
| | | | | | | Col6a2, Plat, Thbs4 |
| c2 | 6 | down | BIOCARTA MAPK PATHWAY | 0.000 | 0.000 | Braf, Fos, Jun, Map3k11, Map4k3, |
| | | | | | | Mapk3, Nkfbia, Rapgef2, Tgfb2 |
| c2 | 6 | down | BIOCARTA PPARA PATHWAY | 0.007 | 0.043 | Cited2, Dusp1, Hspa1a, Jun, |
| | | | | | | Mapk3, Ncoa1, Nfkbia |
| c2 | 6 | down | KEGG B CELL RECEPTOR | 0.000 | 0.012 | Cd81, Fos, Jun, Mapk3, Nfkbia, |
| | | | SIGNALING PATHWAY | | | Pik3r3, Sos2 |
| c2 | 6 | down | KEGG CELL CYCLE | 0.000 | 0.005 | Cdkn1a, Cdkn1c, Gadd45b, |
| | | | | | | Gadd45g, Tgfb2, Zbtb17 |
| c2 | 6 | down | KEGG CHRONIC MYELOID | 0.000 | 0.003 | Braf, Cdkn1a, Ctbp2, Mapk3, |
| | | | LEUKEMIA | | | Mecom, Nfkbia, Pik3r3, Tgfb2, |
| | | | | | | Tgfbr2 |
| c2 | 6 | down | KEGG COLORECTAL CANCER | 0.000 | 0.007 | Braf, Fos, Jun, Mapk3, Pik3r3, |
| | | | | | | Tgfb2, Tgfbr2 |
| c2 | 6 | down | KEGG MAPK SIGNALING | 0.000 | 0.000 | Atf4, Ddit3, Dusp1, Fgf13, Fos, |
| | | | PATHWAY | | | Gadd45b, Gadd45g, Hspa1a, |
| | | | | | | Hspa1b, Hspa8, Hspb1, Jun, Jund, |
| | | | | | | Map3k11, Map4k3, Mapk3, |
| | | | | | | Mecom, Nr4a1, Pdgfb, Ptprr, |
| | | | | | | Rapgef2, Tgfb2, Tgfbr2 |
| c2 | 6 | down | KEGG MELANOMA | 0.007 | 0.023 | Cdkn1a, Fgf13, Mapk3, Pdgfb, |
| | | | | | | Pdgfd, Pik3r3 |
| c2 | 6 | down | KEGG NEUROTROPHIN | 0.004 | 0.023 | Atf4, Braf, Jun, Mapk3, Nfkbia, |
| | | | SIGNALING PATHWAY | | | Pik3r3 |
| c2 | 6 | down | KEGG NOTCH SIGNALING | 0.005 | 0.046 | Ctbp2, Dll4, Dtx3l, Hes1, Jag1 |
| | | | PATHWAY | | | |
| c2 | 6 | down | KEGG PROSTATE CANCER | 0.000 | 0.004 | Atf4, Cdkn1a, Insrr, Mapk3, |
| | | | | | | Nfkbia, Pdgfb, Pdgfd, Pik3r3, |
| | | | | | | Srd5a2 |
| c2 | 6 | down | KEGG RIG I LIKE RECEPTOR | 0.002 | 0.044 | Irf7, Isg15, Mapk12, Nfkbia |
| | | | SIGNALING PATHWAY | | | |
| c2 | 6 | down | KEGG SPLICEOSOME | 0.000 | 0.000 | Ddx5, Hspa1a, Hspa1b, Hspa8, |
| | | | | | | Ncbp2, Srsf2, Srsf7, Tra2b |
| c2 | 6 | down | KEGG TOLL LIKE RECEPTOR | 0.000 | 0.012 | Fos, Irf7, Jun, Mapk12, Mapk3, |
| | | | SIGNALING PATHWAY | | | Nfkbia, Pik3r3 |
| c2 | 6 | down | KEGG T CELL RECEPTOR | 0.000 | 0.005 | Fos, Jun, Mapk12, Mapk3, Nck1, |
| | | | SIGNALING PATHWAY | | | Nfkbia, Pik3r3 |
| c2 | 6 | down | PID AP1 PATHWAY | 0.000 | 0.002 | Atf3, Cyr61, Dusp1, Edn1, Egr1, |
| | | | | | | Fos, Fosb, Jun, Junb, Jund, Nr3c1, |
| | | | | | | Plau |
| c2 | 6 | down | PID AR TF PATHWAY | 0.000 | 0.018 | Dnaja1, Egr1, Jun, Nr3c1, Pou2f1, |
| | | | | | | Smarca2 |
| c2 | 6 | down | PID ATF2 PATHWAY | 0.000 | 0.004 | Atf3, Ddit3, Dusp1, Fos, Hes1, |
| | | | | | | Jun, Junb, Jund, Mapk3, Plau, |
| | | | | | | Pou2f1, Socs3, Tgfb2 |
| c2 | 6 | down | PID BETA CATENIN NUC | 0.006 | 0.025 | Ar, Cyr61, Id2, Jun, Klf4 |
| | | | PATHWAY | | | |
| c2 | 6 | down | PID ERBB1 DOWNSTREAM | 0.004 | 0.025 | Dusp1, Egr1, Fos, Jun, Ksr1, |
| | | | PATHWAY | | | Mapk3, Pik3r3, Zfp36 |
| c2 | 6 | down | PID FRA PATHWAY | 0.000 | 0.043 | Atf4, Jun, Junb, Jund, Plau |
| c2 | 6 | down | PID IL12 2PATHWAY | 0.000 | 0.047 | B2m, Fos, Gadd45b, Gadd45g, |
| | | | | | | Hlx, Jak2, Socs1 |
| c2 | 6 | down | PID IL2 1PATHWAY | 0.002 | 0.041 | Fos, Jun, Mapk3, Socs1, Socs3 |
| c2 | 6 | down | PID MET PATHWAY | 0.010 | 0.022 | Arf6, Egr1, Eif4e, Jun, Mapk3, |
| | | | | | | Nck1, Snai1 |
| c2 | 6 | down | PID PDGFRB PATHWAY | 0.000 | 0.006 | Fos, Jun, Jund, Lrp1, Mapk3, |
| | | | | | | Nck1, Pdgfb, Pik3r3, Slc9a3r2, |
| | | | | | | Sphk1 |
| c2 | 6 | down | PID RB 1PATHWAY | 0.000 | 0.031 | Brd2, Cbx4, Cdkn1a, Jun, Taf1, |
| | | | | | | Tgfb2 |
| c2 | 6 | down | PID REG GR PATHWAY | 0.000 | 0.004 | Cdkn1a, Egr1, Fos, Irf1, Jun, |
| | | | | | | Mapk3, Nr3c1, Nr4a1, Pou2f1, |
| | | | | | | Sgk1 |
| c2 | 6 | down | REACTOME ACTIVATED TLR4 | 0.000 | 0.002 | App, Fos, Irf7, Jun, Mapk3, |
| | | | SIGNALLING | | | Mapk7, Nfkbia, Nod1 |
| c2 | 6 | down | REACTOME CYTOKINE | 0.000 | 0.000 | B2m, Egr1, Eif4e, Gbp7, Ifitm3, |
| | | | SIGNALING IN IMMUNE | | | Irf1, Irf2, Irf7, Irf9, Isg15, Mapk3, |
| | | | SYSTEM | | | Nod1, Pik3r3, Psmb8, Socs1, |
| | | | | | | Socs3, Vcam1, Xaf1 |
| c2 | 6 | down | REACTOME DOWNSTREAM | 0.006 | 0.040 | Cdkn1a, Nfkbia, Nr4a1, Psmb10, |
| | | | SIGNALING EVENTS OF B | | | Psmb8, Psmb9, Them4 |
| | | | CELL RECEPTOR BCR | | | |
| c2 | 6 | down | REACTOME FACTORS | 0.000 | 0.000 | Ak3, Cables2, H3f3b, Irf1, Irf2, |
| | | | INVOLVED IN | | | Irf7, Klc1, Rab5a, Vps45 |
| | | | MEGAKARYOCYTE | | | |
| | | | DEVELOPMENT AND | | | |
| | | | PLATELET PRODUCTION | | | |
| c2 | 6 | down | REACTOME IMMUNE SYSTEM | 0.000 | 0.023 | App, B2m, Cd81, Cdkn1a, Ctso, |
| | | | | | | Dynll1, Egr1, Fos, Gbp7, Hspa5, |
| | | | | | | Ifitm3, Irf1, Irf2, Irf7, Irf9, Isg15, |
| | | | | | | Jun, Klc1, Mapk3, Nck1, Nfkbia, |
| | | | | | | Nod1, Nr4a1, Pik3r3, Psmb10, |
| | | | | | | Psmb8, Psmb9, Sar1b, Socs1, |
| | | | | | | Socs3, Tap2, Vcam1, Xaf1 |
| c2 | 6 | down | REACTOME INTERFERON | 0.000 | 0.000 | Egr1, Ifitm3, Irf1, Irf2, Irf6, Irf7, |
| | | | ALPHA BETA SIGNALING | | | Irf9, Isg15, Psmb8, Socs1, Socs3, |
| | | | | | | Xaf1 |
| c2 | 6 | down | REACTOME INTERFERON | 0.000 | 0.031 | B2m, Gbp5, Gbp7, Irf1, Irf2, Irf7, |
| | | | GAMMA SIGNALING | | | Irf9, Socs1, Socs3, Vcam1 |
| c2 | 6 | down | REACTOME INTERFERON | 0.000 | 0.000 | B2m, Egr1, Eif4e, Gbp7, Ifitm3, |
| | | | SIGNALING | | | Irf1, Irf2, Irf7, Irf9, Isg15, Mapk3, |
| | | | | | | Psmb8, Socs1, Socs3, Vcam1, |
| | | | | | | Xaf1 |
| c2 | 6 | down | REACTOME METABOLISM OF | 0.000 | 0.003 | Anp32a, Cnot6, Eif4e, Hspa1b, |
| | | | MRNA | | | Hspa8, Hspb1, Psmb10, Psmb8, |
| | | | | | | Psmb9, Zfp36, Zfp36l1 |
| c2 | 6 | down | REACTOME METABOLISM OF | 0.000 | 0.008 | Anp32a, Cnot6, Eif4e, Hspa1b, |
| | | | RNA | | | Hspa8, Hspb1, Psmb10, Psmb8, |
| | | | | | | Psmb9, Zfp36, Zfp36l1 |
| c2 | 6 | down | REACTOME MYD88 MAL | 0.000 | 0.004 | App, Fos, Jun, Mapk3, Mapk7, |
| | | | CASCADE INITIATED ON | | | Nfkbia, Nod1 |
| | | | PLASMA MEMBRANE | | | |
| c2 | 6 | down | REACTOME NFKB AND MAP | 0.000 | 0.006 | App, Fos, Jun, Mapk3, Mapk7, |
| | | | KINASES ACTIVATION | | | Nfkbia, Nod1 |
| | | | MEDIATED BY TLR4 | | | |
| | | | SIGNALING REPERTOIRE | | | |
| c2 | 6 | down | REACTOME NUCLEAR | 0.000 | 0.043 | Nr3c1, Nr4a1, Rarg, Rora, Thra |
| | | | RECEPTOR TRANSCRIPTION | | | |
| | | | PATHWAY | | | |
| c2 | 6 | down | REACTOME REGULATION OF | 0.000 | 0.005 | Anp32a, Hspa1b, Hspa8, Hspb1, |
| | | | MRNA STABILITY BY | | | Psmb10, Psmb8, Psmb9, Zfp36, |
| | | | PROTEINS THAT BIND AU | | | Zfp36l1 |
| | | | RICH ELEMENTS | | | |
| c2 | 6 | down | REACTOME RIG I MDA5 | 0.002 | 0.023 | App, Irf1, Irf2, Irf7, Isg15, Nfkbia, |
| | | | MEDIATED INDUCTION OF IFN | | | Ube2k |
| | | | ALPHA BETA PATHWAYS | | | |
| c2 | 6 | down | REACTOME SIGNALING BY | 0.009 | 0.044 | Cdkn1a, Kitl, Mapk3, Nr4a1, |
| | | | SCF KIT | | | Pik3r3, Socs1, Them4, Yes1 |
| c2 | 6 | down | REACTOME SIGNALING BY | 0.008 | 0.021 | Calm1, Cdkn1a, Nck1, Nfkbia, |
| | | | THE B CELL RECEPTOR BCR | | | Nr4a1, Psmb10, Psmb8, Psmb9 |
| c2 | 6 | down | REACTOME TRAF6 MEDIATED | 0.000 | 0.004 | App, Fos, Jun, Mapk3, Mapk7, |
| | | | INDUCTION OF NFKB AND | | | Nfkbia, Nod1 |
| | | | MAP KINASES UPON TLR7 8 | | | |
| | | | OR 9 ACTIVATION | | | |
| c2 | 6 | down | REACTOME TRANSCRIPTION | 0.007 | 0.045 | Cdk9, H3f3b, Mapk3, Polr2l, |
| | | | | | | Polrmt, Pou2f1, Srrm1, Srsf2, |
| | | | | | | Srsf7, Ssb, Taf1 |
| c2 | 6 | down | REACTOME TRIF MEDIATED | 0.000 | 0.005 | App, Fos, Irf7, Jun, Mapk3, |
| | | | TLR3 SIGNALING | | | Mapk7, Nfkbia, Nod1 |
| c2 | 6 | down | REACTOME UNFOLDED | 0.000 | 0.003 | Atf3, Atf4, Dctn1, Ddit3, Dnajb9, |
| | | | PROTEIN RESPONSE | | | Hspa5, Pdia6 |
| c2 | 6 | up | BIOCARTA AMI PATHWAY | 0.000 | 0.007 | Col4a1, Col4a2, F2r, Tfpi |
| c2 | 6 | up | BIOCARTA FIBRINOLYSIS | 0.002 | 0.028 | F2r, Serpine1 |
| | | | PATHWAY | | | |
| c2 | 6 | up | BIOCARTA INTRINSIC | 0.000 | 0.013 | Col4a1, Col4a2, F2r |
| | | | PATHWAY | | | |
| c2 | 6 | up | BIOCARTA VITCB PATHWAY | 0.000 | 0.041 | Col4a1, Col4a2 |
| c2 | 6 | up | KEGG PROXIMAL TUBULE | 0.003 | 0.045 | Aqp1, Glud1 |
| | | | BICARBONATE | | | |
| | | | RECLAMATION | | | |
| c2 | 6 | up | NABA COLLAGENS | 0.000 | 0.028 | Col15a1, Col27a1, Col4a1, |
| | | | | | | Col4a2, Col5a1, Col5a3 |
| c2 | 6 | up | PID ALK2 PATHWAY | 0.002 | 0.049 | Fkbp1a, Smad1 |
| c2 | 6 | up | REACTOME COLLAGEN | 0.000 | 0.021 | Col15a1, Col27a1, Col4a1, |
| | | | FORMATION | | | Col4a2, Col5a1, Col5a3, Plod1, |
| | | | | | | Ppib |
| c2 | 6 | up | REACTOME EXTRACELLULAR | 0.000 | 0.010 | Col15a1, Col27a1, Col4a1, |
| | | | MATRIX ORGANIZATION | | | Col4a2, Col5a1, Col5a3, Mmp14, |
| | | | | | | Plod1, Ppib |
| c2 | 6 | up | REACTOME INWARDLY | 0.000 | 0.045 | Abcc9, Gngt2, Kcnj8 |
| | | | RECTIFYING K CHANNELS | | | |
| c2 | 6 | up | REACTOME NCAM1 | 0.000 | 0.010 | Col4a1, Col4a2, Prnp |
| | | | INTERACTIONS | | | |
| c2 | 6 | up | REACTOME REGULATION OF | 0.000 | 0.007 | Igfbp3, Igfbp5 |
| | | | INSULIN LIKE GROWTH | | | |
| | | | FACTOR IGF ACTIVITY BY | | | |
| | | | INSULIN LIKE GROWTH | | | |
| | | | FACTOR BINDING PROTEINS | | | |
| | | | IGFBPS | | | |
| c2 | 6 | up | REACTOME SRP DEPENDENT | 0.000 | 0.049 | Rpn2, Sec11a, Ssr2, Ssr4, Tram1 |
| | | | COTRANSLATIONAL PROTEIN | | | |
| | | | TARGETING TO MEMBRANE | | | |
| c2 | 7 | up | BIOCARTA P53HYPOXIA | 0.000 | 0.011 | Akt1, Cdkn1a, Ep300, Hspa1a |
| | | | PATHWAY | | | |
| c2 | 7 | up | BIOCARTA P53 PATHWAY | 0.000 | 0.012 | Ccnd1, Cdkn1a, Timp3 |
| c2 | 7 | up | NABA ECM REGULATORS | 0.000 | 0.041 | Adam9, Fam20a, Fam20c, Hpse2, |
| | | | | | | Htra1, Htra3, Kng2, Lox, Loxl2, |
| | | | | | | Mmp11, Pappa2, Serpine1, |
| | | | | | | Serping1, Sulf1, Tgm2, Timp1, |
| | | | | | | Timp2, Timp3 |
| c2 | 7 | up | PID ATF2 PATHWAY | 0.000 | 0.001 | Ccnd1, Csrp2, Dusp1, Ep300, Jun, |
| | | | | | | Junb, Jund, Mapk3, Nf1, Pdgfra, |
| | | | | | | Socs3 |
| c2 | 7 | up | PID HIF2PATHWAY | 0.000 | 0.010 | Bhlhe40, Cited2, Ep300, Kdr, |
| | | | | | | Serpine1, Vhl |
| c2 | 7 | up | PID UPA UPAR PATHWAY | 0.000 | 0.000 | Itgav, Itgb5, Lrp1, Pdgfrb, |
| | | | | | | Serpine1 |
| c2 | 7 | up | REACTOME SIGNALING BY | 0.000 | 0.013 | Akt1, Cdkn1a, Col6a1, Col6a3, |
| | | | PDGF | | | Mapk3, Pdgfc, Pdgfra, Pdgfrb, |
| | | | | | | Plcg1, Prkaca, Prkar2a, Spp1, |
| | | | | | | Stat1, Thbs4, Ywhab |
| c2 | 8 | down | BIOCARTA HDAC PATHWAY | 0.000 | 0.025 | Calm1, Calm2, Igf1, Mapk7, |
| | | | | | | Mef2a, Mef2c |
| c2 | 8 | down | REACTOME ANTIGEN | 0.000 | 0.044 | Calr, Canx, Hspa5, Pdia3, Sec31a |
| | | | PRESENTATION FOLDING | | | |
| | | | ASSEMBLY AND PEPTIDE | | | |
| | | | LOADING OF CLASS I MHC | | | |
| c2 | 8 | up | PID P53 DOWNSTREAM | 0.000 | 0.001 | Bcl2, Btg2, Ddit4, Dusp1, |
| | | | PATHWAY | | | Gadd45a, Map4k4, Ndrg1, |
| | | | | | | Serpine1, Spp1 |
| c3 | 0 | down | AACTGAC MIR223 | 0.003 | 0.048 | Cdk17, Crim1, Hhex, Igf1r, Il6st, |
| | | | | | | Map1b, Qk, Rhob, Son, Tspan7 |
| c3 | 0 | down | ACTGTAG MIR139 | 0.000 | 0.042 | Auts2, Dhx15, Fos, Gdf10, Jdp2, |
| | | | | | | Jun, Prpf4b, Top1 |
| c3 | 0 | down | AGCTCCT MIR28 | 0.000 | 0.038 | Clk3, Crim1, Csf1, Gpm6a, Lbh, |
| | | | | | | Qk, Sarm1, Srsf5 |
| c3 | 0 | down | CEBPB 02 | 0.000 | 0.048 | Asah2, Csnk1e, Fgf14, H3f3b, |
| | | | | | | Map2k6, Map4k4, Nfil3, Nfkbia, |
| | | | | | | Nfkbiz, Pctp, Pdgfb, Pparg, |
| | | | | | | Rbm39, Rhob, Smarca2, Sox10, |
| | | | | | | Tob1, Top1, Trib1 |
| c3 | 0 | down | CEBP C | 0.000 | 0.044 | Cebpb, Crim1, Epc2, H3f3b, Jun, |
| | | | | | | Map4k4, Msl3, Myl1, Nfkbia, |
| | | | | | | Rbm39, Sat1, Tspan7 |
| c3 | 0 | down | CTACTGT MIR199A | 0.000 | 0.027 | Bmp2, Cdk17, Cited2, Fos, |
| | | | | | | Gpm6a, Lifr, Ptx3, Qk, Rbm39, |
| | | | | | | Rlim, Smarca2, Top1, Wsb1, |
| | | | | | | Ythdc1 |
| c3 | 0 | down | DR4 Q2 | 0.000 | 0.028 | Adamts4, Cdk17, Id2, Jarid2, Lipg, |
| | | | | | | Map4k4, Nfkbia, Nr5a2, Pdgfb, |
| | | | | | | Rbm39, Sik1, Son, Upf2, Zfp513 |
| c3 | 0 | down | FOXD3 01 | 0.000 | 0.021 | Bambi, Cited2, Crim1, Cxadr, Id2, |
| | | | | | | Ier3, Junb, Msl3, Tbl1xr1, Tob1 |
| c3 | 0 | down | GCATTTG MIR105 | 0.000 | 0.034 | Alkbh5, Coq10b, H3f3b, Itm2b, |
| | | | | | | Map1b, Prpf4b, Son, Tbl1xr1, |
| | | | | | | Tiparp, Tra2b |
| c3 | 0 | down | GTCGATC MIR3695P | 0.011 | 0.041 | Dhx15, Id4, Macf1 |
| c3 | 0 | down | GTGACTT MIR224 | 0.000 | 0.034 | Epc2, Fosb, H3f3b, Itm2b, Map1b, |
| | | | | | | Plpp3, Prpf4b, Qk, Tra2b, Wsb1 |
| c3 | 0 | down | HFH4 01 | 0.000 | 0.041 | Bambi, Cited2, Crim1, H3f3b, Id1, |
| | | | | | | Id2, Map4k4, Msl3, Myo10, Pparg, |
| | | | | | | Tppp3 |
| c3 | 0 | down | KCCGNSWTTT UNKNOWN | 0.003 | 0.037 | Ddx5, Dhx15, Jarid2, Rbm39, |
| | | | (SEQ ID NO: 6) | | | Taf15, Tra2b, Ubc |
| c3 | 0 | down | OCT1 B | 0.000 | 0.049 | Cdh13, Csnk1e, Csrnp1, Gpm6a, |
| | | | | | | H3f3b, Jund, Lipg, Rgl1, Rhob |
| c3 | 0 | down | STAT5A 04 | 0.000 | 0.047 | Cited2, Fgf14, Gadd45g, Gria1, |
| | | | | | | Nr5a2, Rbm39, Slc50a1, Ssbp2 |
| c3 | 0 | down | TTAYRTAA E4BP4 01 | 0.000 | 0.000 | Cdkl1, Ciart, Crim1, Csnk1e, |
| | | | | | | Ddx5, Gpm6a, Id4, Il6st, Mylk, |
| | | | | | | Nfil3, Nmt2, Pparg, Prnp, Rgl1, |
| | | | | | | Sh3gl2, Tob1, Tspan7 |
| c3 | 0 | down | TTGGAGA MIR5155P MIR519E | 0.000 | 0.050 | Auts2, Btg2, Epc2, Fam84b, Pum1, |
| | | | | | | Sertad1, Tacc1, Zfp36l1 |
| c3 | 0 | down | TTTGTAG MIR520D | 0.000 | 0.044 | Alkbh5, Atf4, Cand1, Cited2, |
| | | | | | | Dclk1, Fam84b, Gadd45a, Gpm6a, |
| | | | | | | H3f3b, Maff, Nr5a2, Plekhh3, |
| | | | | | | Prpf4b, Pum1, Qk, Rlim, Sik1, |
| | | | | | | Son, Srsf5, Tbl1xr1, Tiparp |
| c3 | 0 | down | YYCATTCAWW UNKNOWN | 0.000 | 0.047 | Ap1b1, B3gnt6, Dclk1, Ddx5, |
| | | | (SEQ ID NO: 7) | | | Ets2, Fosb, Gpm6a, Id1, Igf1r, |
| | | | | | | Maff, Plpp3, Rgs3, Tob1 |
| c3 | 11 | down | GCCATNTTG YY1 Q6 | 0.000 | 0.009 | Ap4s1, Atrx, Bambi, Ccnd2, |
| | | | | | | Ddx17, Elk3, Foxp1, Gpbp1, |
| | | | | | | Hspa8, Lima1, Pcf11, Polr2a, |
| | | | | | | Prickle3, Prpf38b, Snx17, Syne2, |
| | | | | | | Tra2a |
| c3 | 11 | down | TTCNRGNNNNTTC HSF Q6 | 0.000 | 0.026 | Ahsa1, Ahsa2, Cacna1g, Chordc1, |
| | | | (SEQ ID NO: 8) | | | Ddx17, Dnaja1, Edn1, Eif4a2, |
| | | | | | | Fosb, Foxp1, Hspa1a, Hspa8 |
| c3 | 12 | down | GTCGATC MIR3695P | 0.038 | 0.018 | Id4, Macf1 |
| c3 | 12 | down | HSF Q6 | 0.000 | 0.015 | Alkbh5, Atf3, Cryab, Eif4a2, |
| | | | | | | Jmjd6, P4ha1, Ppp2r2a, Serpinh1, |
| | | | | | | St13, Tsc22d2, Zbtb25 |
| c3 | 12 | down | STAT3 02 | 0.000 | 0.012 | Azin1, Ddit3, Eif4e, Fosb, Irf1, |
| | | | | | | Myt1, Shox2, Tcf712 |
| c3 | 12 | down | TTCNRGNNNNTTC HSF Q6 | 0.000 | 0.000 | Cryab, Eif4a2, Fosb, Hspa8, |
| | | | (SEQ ID NO: 8) | | | Jmjd6, Myl1, Serpinh1, St13, |
| | | | | | | Ywhae |
| c3 | 15 | down | ACTGCCT MIR34B | 0.003 | 0.044 | Ank2, Arid1b, Btg2, Elavl1, Lrp6, |
| | | | | | | Mtdh, Nfat5, Papola, Phactr1, |
| | | | | | | Ppp1r10, Uhrf2, Vamp2 |
| c3 | 15 | down | ATACTGT MIR144 | 0.000 | 0.004 | Acbd3, Ank2, Clk1, Fosb, Mtpn, |
| | | | | | | Nrp2, Pafah1b1, Pim1, Smpd3, |
| | | | | | | Srsf10, Usp42 |
| c3 | 15 | down | CIZ 01 | 0.000 | 0.013 | Ckap4, Coa3, Col3a1, Crk, H3f3b, |
| | | | | | | Hoxb7, Id2, Mmp2, Ppp1r10, |
| | | | | | | Prrx1, Tcf4, Tnks1bp1, Tubgcp4 |
| c3 | 15 | down | E2F1 Q4 | 0.008 | 0.037 | Eva1b, Fcf1, Hoxb9, Id3, Lgals1, |
| | | | | | | Luc7l2, Nop10, Nrp2, Prkcsh, |
| | | | | | | Ypel5 |
| c3 | 15 | down | KCCGNSWTTT UNKNOWN | 0.000 | 0.047 | Crk, Eif4a2, Irx5, Pafah1b1, |
| | | | (SEQ ID NO: 6) | | | Syncrip, Tra2b |
| c3 | 15 | down | MEIS1 01 | 0.000 | 0.046 | Ank2, Ash1l, Ckap4, Copz2, Ikzf5, |
| | | | | | | Nrp1, Pafah1b1, Runx1t1, |
| | | | | | | Slc25a25, Smpd3, Tnks1bp1, |
| | | | | | | Tnpo1 |
| c3 | 15 | down | NFMUE1 Q6 | 0.000 | 0.041 | Ash1l, Clk1, Cox20, Fkrp, |
| | | | | | | Hnrnph3, Maea, Prkcsh, Sfpq, |
| | | | | | | Tcf4, Tra2b, Vamp2 |
| c3 | 15 | down | YY1 Q6 | 0.006 | 0.031 | Ash1l, Fkrp, Hnrnph3, Ier2, Maea, |
| | | | | | | Pnrc2, Prkcsh, Sfpq, Tcf4, Tra2b |
| c3 | 1 | down | AATGTGA MIR23A MIR23B | 0.000 | 0.020 | Adamts6, Azin1, Ccl7, Cxcl12, |
| | | | | | | Dennd1b, Ebf3, Egln2, Fosb, Gja1, |
| | | | | | | Grem1, Irf1, Kitl, Marcksl1, Npr3, |
| | | | | | | Prpf4b, Sgk1, Top1, Trib1, Wsb1 |
| c3 | 1 | down | AHRARNT 01 | 0.000 | 0.045 | Ctgf, Dmc1, Eif4a2, Ghr, Hes1, |
| | | | | | | Il7, Klf10, Nipbl, Pax6, Rbm6, |
| | | | | | | Runx1, Sez6, Sgk1, Wsb1, Zeb2 |
| c3 | 1 | down | ATACTGT MIR144 | 0.000 | 0.049 | Cdhl1, Clk1, Cxcl12, Fbxl3, Fosb, |
| | | | | | | Pappa, Pim1, Son, Trib1, Zeb2 |
| c3 | 1 | down | ATF 01 | 0.000 | 0.045 | Atp6v0c, Fosb, Irf2bpl, Ppp1r15a, |
| | | | | | | Sgk1, Slc25a25, Son, St13, Top1, |
| | | | | | | Tra2b, Trib1, Tsc22d2, Zfand5 |
| c3 | 1 | down | CCTGCTG MIR214 | 0.000 | 0.046 | 1810013L24Rik, Ammecr1l, |
| | | | | | | Arhgap31, Bace2, Baz2a, Csf1, |
| | | | | | | Cxxc5, Egln2, Gpr85, Hmgn3, |
| | | | | | | Igdcc3, Lmod1, Med19, Mid1, |
| | | | | | | Mroh7, Ndufaf5, Pappa, Pim1, |
| | | | | | | Ppm1a, Ppme1, Shisa6, Snhg11, |
| | | | | | | Srgap3, Tbc1d25, Tmem248 |
| c3 | 1 | down | CEBPDELTA Q6 | 0.000 | 0.046 | Ccnl1, Dtna, Efemp1, Fosb, Gpr85, |
| | | | | | | Gtf3c2, Mid1, Nnmt, Pik3r1, |
| | | | | | | Ppm1a, Pten, Rbfox1, Rbm39, |
| | | | | | | Rora, Trib1, Zeb2 |
| c3 | 1 | down | CHX10 01 | 0.000 | 0.049 | Adamts17, Bace2, Bambi, Bmp4, |
| | | | | | | Btbd3, Fosb, Gpr158, Impg2, |
| | | | | | | Npr3, Ntrk1, Pax6, Pdzm4, Plxna2, |
| | | | | | | Slc26a7, Vsnl1, Ypel4 |
| c3 | 1 | down | EVI1 03 | 0.002 | 0.047 | Il7, Rbm39, Rspo2, Ube2k |
| c3 | 1 | down | EVI1 05 | 0.000 | 0.049 | Bace2, Il7, Mid1, Ogn, Rbm39, |
| | | | | | | Rspo2, Ube2k |
| c3 | 1 | down | GTAAACC MIR2995P | 0.002 | 0.040 | Cpeb2, H3f3b, Sgk1, Srsf7, Tiparp |
| c3 | 1 | down | KCCGNSWTTT UNKNOWN | 0.000 | 0.046 | Csnk1a1, Cxxc5, Ddx5, Dmtf1, |
| | | | (SEQ ID NO: 6) | | | Eif4a2, Ilf3, Nhs, Nipbl, Rbm39, |
| | | | | | | Srrm1, Tra2b |
| c3 | 1 | down | KTGGYRSGAA UNKNOWN | 0.002 | 0.031 | Cntfr, Mis12, Myc, Pim1, Srsf7, |
| | | | | | | Zmym4 |
| c3 | 1 | down | MYCMAX 01 | 0.000 | 0.038 | Adamts17, Atf7ip, Bcas3, Bmp4, |
| | | | | | | Bok, Dazap1, Diablo, Fgf6, Foxo3, |
| | | | | | | Gadd45g, Gja1, Hnrnph3, Hoxa11, |
| | | | | | | Hoxc11, Ilf3, Mxd4, Per1, Prdx4, |
| | | | | | | Prps1, Rspo2, Rtn4rl2, Snx5, Syt3, |
| | | | | | | Top1, Trib1, Ubxn10 |
| c3 | 1 | down | NFKB Q6 01 | 0.000 | 0.049 | Ebf1, Grem1, Icam1, Il7, Nfat5, |
| | | | | | | Nfkbia, Pappa, Top1, Vcam1 |
| c3 | 1 | down | OCT1 07 | 0.002 | 0.044 | Bace2, Btbd3, Dtna, Fgfr2, Hdac9, |
| | | | | | | Immp1l, Mid1, Pdzm4, Pou3f4, |
| | | | | | | Runx1, Scoc |
| c3 | 1 | down | PAX5 02 | 0.004 | 0.044 | Dtx1, Pim1, Zeb2 |
| c3 | 1 | down | RNTCANNRNNYNATTW | 0.000 | 0.024 | Chrdl1, Nfat5, Pdzm4, Zeb2 |
| | | | UNKNOWN (SEQ ID NO: 9) | | | |
| c3 | 1 | down | SPZ1 01 | 0.000 | 0.040 | Adamts9, Ahcyl2, Cntfr, Ebf1, Fst, |
| | | | | | | Grem1, Hnrnpll, Ilf3, Mylk, |
| | | | | | | Ptchd1, Spry4, Srrm1, Vcam1 |
| c3 | 1 | down | SREBP1 02 | 0.000 | 0.043 | Cebpb, Grem1, Hyal1, Notch2, |
| | | | | | | Ptchd1 |
| c3 | 1 | down | TAATGTG MIR323 | 0.000 | 0.045 | Adamts6, Cxcl12, Ebf3, Gja1, |
| | | | | | | Irf2bpl, Marcksl1, Nfat5, Npr3, |
| | | | | | | Top1, Zeb2 |
| c3 | 1 | down | TCTAGAG MIR517 | 0.002 | 0.037 | Fst, Hmgn3, Ilf3, Pappa |
| c3 | 2 | down | ACAWYAAAG UNKNOWN | 0.000 | 0.014 | Abr, Ank, Arhgap20, Cask, Gpc4, |
| | | | | | | Pam, S100a3, Shox2 |
| c3 | 2 | down | ACCTGTTG UNKNOWN | 0.000 | 0.016 | Abr, Arl4a, Atxn1, Chst11, Erf, |
| | | | | | | Npas2, Nr3c2, Nr4a2, Sec24d, |
| | | | | | | Shox2 |
| c3 | 2 | down | ACTGTAG MIR139 | 0.001 | 0.005 | Atxn1, Auts2, Btg3, Cpeb4, |
| | | | | | | Eif4g2, Fos, Hoxa9, Pde3a, Piezo1, |
| | | | | | | Ppp2ca, Socs2, Top1, Tspan3, |
| | | | | | | Ube2f, Ywhag, Ywhaq |
| c3 | 2 | down | ACTGTGA MIR27A MIR27B | 0.000 | 0.016 | Ak2, Atp2b1, Cbfb, Cemip, |
| | | | | | | Colgalt2, Cpeb4, Ell2, Fam133b, |
| | | | | | | Fosb, Foxp4, H3f3b, Id2, Jmjd1c, |
| | | | | | | Kcna6, Kctd4, Lbh, Mdfi, Npepps, |
| | | | | | | Nr2f6, Nrep, Nrip1, Nrp2, Pde3a, |
| | | | | | | Rlim, Rnd3, Rnf139, Tmed5, |
| | | | | | | Tmem9b, Ube2f, Ube2n, Vangl1, |
| | | | | | | Vangl2, Wsb1 |
| c3 | 2 | down | AGCATTA MIR155 | 0.003 | 0.021 | Atp2b1, Cd47, Cebpb, Ell2, Hif1a, |
| | | | | | | Hivep2, Midn, Rab1a, Ski, Socs1 |
| c3 | 2 | down | AGGAAGC MIR5163P | 0.004 | 0.025 | Baiap2, Ier5, Pcnp, Pitpnc1, |
| | | | | | | Rbfox1, Runx3, S100b, Vasn, |
| | | | | | | Xbp1, Zfp385b |
| c3 | 2 | down | AHR 01 | 0.000 | 0.023 | Angptl2, Dynll1, H1f0, Hmg20b, |
| | | | | | | Kcna6, Picalm, Rtn4, Runx3 |
| c3 | 2 | down | AHR Q5 | 0.000 | 0.028 | 4921524J17Rik, Bhlhe41, Dstn, |
| | | | | | | Fosb, Gadd45g, Hnrnpdl, Irf2bpl, |
| | | | | | | Mbd3, Ndufa13, Pitpnc1, Rhoa, |
| | | | | | | Rtn4, Smg6, Vegfa, Vldlr |
| c3 | 2 | down | AP1FJ Q2 | 0.003 | 0.029 | Cask, Col27a1, Dstn, Fos, Id1, |
| | | | | | | Jmjd1c, Lmna, Mdfi, Pitpnc1, |
| | | | | | | Ppp2ca, Rtn4, Slc20a1, Slc35e4, |
| | | | | | | Smap2, Stx17, Synpo, Tgfbr2, |
| | | | | | | Tob1, Tuba1c, Zfp385b |
| c3 | 2 | down | AP1 C | 0.000 | 0.031 | Aig1, Btg3, Clic1, Cpne8, Emp1, |
| | | | | | | Gadd45g, Iqsec1, Lmna, Mdfi, |
| | | | | | | Pea15a, Pitpnc1, Ppp1r15a, |
| | | | | | | Ppp2ca, Prr7, Rela, Sec24d, Tob1, |
| | | | | | | Vapa, Yif1a, Zfand5, Zfp385b |
| c3 | 2 | down | AP1 Q2 | 0.000 | 0.022 | Baiap2, Cask, Col27a1, Dstn, Fos, |
| | | | | | | Id1, Jmjd1c, Lmna, Mdfi, Pitpnc1, |
| | | | | | | Ppp2ca, Slc31a1, Slc35e4, Smap2, |
| | | | | | | Stx17, Tgfbr2, Tob1, Tuba1c, |
| | | | | | | Txn1, Zfp385b |
| c3 | 2 | down | AP1 Q6 | 0.000 | 0.043 | Abhd2, Cask, Cdkn1a, Col27a1, |
| | | | | | | Dstn, Eif4e, Lmna, Mdfi, Pea15a, |
| | | | | | | Pitpnc1, Ppp2ca, Prr7, Rtn4, |
| | | | | | | Slc35e4, Spats2, Stx17, Tgfbr2, |
| | | | | | | Trib1, Zfand5, Zfp385b |
| c3 | 2 | down | AP2GAMMA 01 | 0.000 | 0.013 | Angptl2, Calm1, Cpeb4, Cxxc5, |
| | | | | | | Erf, Fkbp14, Hif1a, Hnrnpdl, |
| | | | | | | Hoxc10, Jund, Klf10, Klf13, |
| | | | | | | P4ha1, Slc39a7, Smad7, Tspan17, |
| | | | | | | Zfp362, Znrf2 |
| c3 | 2 | down | AP3 Q6 | 0.000 | 0.023 | Actn4, Bag3, Cask, Cd109, Hdlbp, |
| | | | | | | Hoxc10, Jmjd1c, Jund, Map4k4, |
| | | | | | | Nrep, Phf3, Rora, Sgms2, Slc44a1, |
| | | | | | | Sobp, Sp7, Tnip1, Trim46, Trps1, |
| | | | | | | Unc5c, Whrn, Wwp2 |
| c3 | 2 | down | ATACTGT MIR144 | 0.000 | 0.026 | Atp2b1, Atxn1, Ccdc88a, |
| | | | | | | D5Ertd579e, Eif4g2, Eif5, Ell2, |
| | | | | | | Fosb, Lgr4, Nrp2, Pim1, Pum1, |
| | | | | | | Smpd3, Sobp, Trib1, Wif1, Wtap |
| c3 | 2 | down | ATGTAGC MIR221 MIR222 | 0.004 | 0.031 | Angptl2, Atxn1, Cbfb, Cpne8, Fos, |
| | | | | | | Hoxc10, Midn, Rab1a, Rala, |
| | | | | | | Trps1, Vgll4, Ywhag |
| c3 | 2 | down | ATGTTTC MIR494 | 0.000 | 0.014 | Atf3, Bhlhe40, Chd9, Eif5, H3f3b, |
| | | | | | | Htra1, Irf2bpl, Jund, Rbm39, |
| | | | | | | Tacc1, Tmed2, Trps1, Vps26b, |
| | | | | | | Znrf2 |
| c3 | 2 | down | BRN2 01 | 0.003 | 0.040 | Aifm1, Arf6, Bambi, Bcor, Cbfb, |
| | | | | | | Ctnnbip1, Dusp6, Fos, H3f3b, |
| | | | | | | Inppl1, Nfia, Npas2, Phf3, Pim1, |
| | | | | | | Sat1, Saysd1, Tob1, Txn1, Ubl3, |
| | | | | | | Zfp385b, Zmiz1 |
| c3 | 2 | down | CACCAGC MIR138 | 0.000 | 0.002 | Aff3, Baiap2, Baz1b, Btg1, Cpeb4, |
| | | | | | | H3f3b, Jmjd1c, Mcu, Nsmf, Pde3a, |
| | | | | | | Rhoc, Scn1b, Slc20a1, Smap2, |
| | | | | | | Sobp, Tiparp, Trps1, Zmiz1 |
| c3 | 2 | down | CIZ 01 | 0.000 | 0.002 | BC005537, Borcs6, Ccdc88a, |
| | | | | | | Ckap4, Col27a1, Dlx5, Gadd45g, |
| | | | | | | H3f3b, Id2, Nr4a2, Pax1, Pitpnc1, |
| | | | | | | Stim1, Tob1, Wwp2, Ywhag, |
| | | | | | | Zbtb20, Zmynd8 |
| c3 | 2 | down | CTACTGT MIR199A | 0.001 | 0.024 | Atf3, BC005537, Bzw1, Cpeb4, |
| | | | | | | Cxxc5, Fos, Hoxa9, Nrp2, Plod2, |
| | | | | | | Rbm39, Rlim, Rnf139, Sobp, Srf, |
| | | | | | | Top1, Vegfa, Vldlr, Wsb1 |
| c3 | 2 | down | DBP Q6 | 0.001 | 0.021 | Aff3, Auts2, Bhlhe41, Btg1, |
| | | | | | | Dusp6, Eps8l2, Hs3st3a1, Kctd6, |
| | | | | | | Klf3, Kmt2d, Mgat4b, Nfia, |
| | | | | | | Rab1a, Scube1, Slc29a1, Slc39a7, |
| | | | | | | Stx17, Tfdp2, Tob1, Trib1, |
| | | | | | | Zfp385b |
| c3 | 2 | down | ELF1 Q6 | 0.001 | 0.028 | Clic1, Erf, Hoxc10, Klf13, Lrrc8a, |
| | | | | | | Map4k2, Nfia, Pum1, Rala, Rela, |
| | | | | | | Rlim, Sat1, Stim1 |
| c3 | 2 | down | FOXJ2 02 | 0.000 | 0.021 | Anapc11, Arf6, Atf3, BC005537, |
| | | | | | | Casc4, Dixdc1, Fchsd2, Hoxa9, |
| | | | | | | Id1, Klf3, Nfia, Pde4b, Rbm39, |
| | | | | | | Rora, Slc39a14, Sobp, Tob1 |
| c3 | 2 | down | GCATTTG MIR105 | 0.001 | 0.025 | Camk2g, Dicer1, Eif4g2, F13a1, |
| | | | | | | H3f3b, Map3k2, Slc31a1, Socs5, |
| | | | | | | Socs7, Spats21, Sspn, Tiparp, |
| | | | | | | Tpm3, Tra2b, Zfr |
| c3 | 2 | down | GGATTA PITX2 Q2 | 0.001 | 0.049 | Angptl2, Arf6, Arfip1, Atxn1, |
| | | | | | | BC005537, Btg1, Cpeb4, Cxxc5, |
| | | | | | | Dusp6, Ehf, Gadd45b, H1f0, |
| | | | | | | Iqsec1, Jmjd1c, Jund, Ktn1, |
| | | | | | | Npepps, Pde4b, Pla2g5, Rora, |
| | | | | | | Shox2, Skil, Slc44a1, Slc4a2, |
| | | | | | | Smg6, Socs2, Sp7, Srpx2, Trib1, |
| | | | | | | Trps1, Ubxn4, Znrf2 |
| c3 | 2 | down | GGCNRNWCTTYS UNKNOWN | 0.005 | 0.022 | Ank, Hoxa9, Irf2bpl, Klf10, |
| | | | (SEQ ID NO: 10) | | | Nkain1, Nrep, Smap2 |
| c3 | 2 | down | GGGNNTTTCC NFKB Q6 01 | 0.001 | 0.014 | Ccdc107, Cpeb4, Gata4, Gem, |
| | | | (SEQ ID NO: 11) | | | Ier5, Slc44a1, Slc6a12, Smpd3, |
| | | | | | | Tnip1, Tpm3, Trim47, Upf2 |
| c3 | 2 | down | GTATTAT MIR3693P | 0.005 | 0.049 | Arl2bp, Cask, Cebpb, Eps8l2, |
| | | | | | | Hes1, Ncoa3, Pde4b, Peli1, |
| | | | | | | Trappc8, Trps1, Wsb1, Zfand5, |
| | | | | | | Zfr, Zmynd8 |
| c3 | 2 | down | GTCAGGA MIR378 | 0.000 | 0.049 | Abr, Ccdc88a, Fam133b, Gnaq, |
| | | | | | | Klf9, Tom1l2, Vangl2 |
| c3 | 2 | down | HEN1 01 | 0.001 | 0.028 | Arl4a, Calm2, Cdc42ep3, |
| | | | | | | Cdc42se1, Clint1, Dlx5, Foxp4, |
| | | | | | | Hif1a, Irf2bpl, Jmjd1c, Kcnn2, |
| | | | | | | Klf13, Nr4a2, Rela, Sgms2, Tpm3, |
| | | | | | | Trim46 |
| c3 | 2 | down | HEN1 02 | 0.001 | 0.025 | Arl4a, Calm2, Cdc42ep3, |
| | | | | | | Cdc42se1, Clint1, Eif4g2, Irf2bpl, |
| | | | | | | Kcnn2, Ndufa4, Nr4a2, Ogfod2, |
| | | | | | | Ptgds, Rab1a, Rela, Slc44a1, Sobp, |
| | | | | | | Trim46 |
| c3 | 2 | down | HFH8 01 | 0.001 | 0.025 | Bambi, Col13a1, Cpeb4, Cxxc5, |
| | | | | | | Fstl1, Id2, Kif21a, Klf3, Mxra8, |
| | | | | | | Nr4a2, Rasd1, Rora, Tob1, |
| | | | | | | Tspan17, Zmynd8 |
| c3 | 2 | down | IRF Q6 | 0.000 | 0.015 | Arhgef6, Bcor, Cask, Cpeb4, Ext1, |
| | | | | | | F3, Lsp1, Npepps, Prr7, Rrbp1, |
| | | | | | | Rtn4, Sat1, Slc39a7, Socs1, Tfdp2, |
| | | | | | | Top1, Upf2 |
| c3 | 2 | down | NFAT Q6 | 0.000 | 0.015 | Arf6, Cdc42se1, Cgrrf1, Cpeb4, |
| | | | | | | Emp1, Gadd45g, Id2, Ift20, Klf13, |
| | | | | | | Map4k4, Plod2, Rhoa, Shox2, |
| | | | | | | Slc6a12, Tfdp2, Trim47, Trps1, |
| | | | | | | Zfp362, Zmynd8 |
| c3 | 2 | down | NFKAPPAB65 01 | 0.000 | 0.022 | Amotl1, Ccdc107, Cdc42se1, Ehf, |
| | | | | | | Gadd45b, Ier3, Ier5, Kcnn2, Mia, |
| | | | | | | Sdc4, Sec63, Slc44a1, Slc6a12, |
| | | | | | | Smoc1, Smpd3, Tnip1, Trim47, |
| | | | | | | Upf2, Ywhaq |
| c3 | 2 | down | NFKB Q6 01 | 0.000 | 0.013 | Actn1, Amotl1, Ccdc107, |
| | | | | | | Cdc42se1, Col27a1, Dusp6, Ehf, |
| | | | | | | Gadd45b, Ier5, Mag, Ptms, Shox2, |
| | | | | | | Slc44a1, Slc6a12, Smoc1, Smpd3, |
| | | | | | | Socs2, Tnip1, Top1, Trim47, Upf2 |
| c3 | 2 | down | RACCACAR AML Q6 | 0.001 | 0.026 | 1700123O20Rik, Amd1, Batf3, |
| | | | | | | Cemip, Cthrc1, Emp1, Fgfr1, |
| | | | | | | Hoxa9, Jmjd1c, Mtx1, Nucb2, |
| | | | | | | Pax1, Pitpnc1, Thbs3, Vasn |
| c3 | 2 | down | STAT5A 02 | 0.007 | 0.048 | Arl4a, Atp5j, Emp1, Fos, Foxa3, |
| | | | | | | Klf4, Klf9, Rbfox1, Sdc1, Sept7, |
| | | | | | | Snx18, Socs2, Trim47, Zfr |
| c3 | 2 | down | STAT5A 03 | 0.000 | 0.021 | Actn1, Amd1, Borcs6, Btg1, Dlx5, |
| | | | | | | Gpc4, Hoxc10, Map4k4, Pim1, |
| | | | | | | Rcan1, Tnfrsf11b, Tob1, Tpm3, |
| | | | | | | Trps1, Vangl1, Zbtb20 |
| c3 | 2 | down | TAAWWATAG RSRFC4 Q2 | 0.001 | 0.023 | Atxn1, Cdkn1a, Cldn14, Hoxa9, |
| | | | | | | Inppl1, Ktn1, Lrrc20, Nrep, Nrp2, |
| | | | | | | Shox2, Trps1, Zfand5, Zfp385b |
| c3 | 2 | down | TATTATA MIR374 | 0.000 | 0.024 | Abr, Actn1, Actn4, Amd1, Arl2bp, |
| | | | | | | Atxn1, Bhlhe40, Ccnl1, Cd47, |
| | | | | | | Cebpb, Cemip, Cpeb4, Dusp6, |
| | | | | | | Eif4g2, Fosb, Hes1, Hivep2, |
| | | | | | | Nr4a2, Pde4b, Peli1, Rala, Tcerg1, |
| | | | | | | Tmem47 |
| c3 | 2 | down | TCF11 01 | 0.004 | 0.045 | Aig1, BC005537, Ccdc88a, Cpeb4, |
| | | | | | | Eif4e, Gpc4, Ift20, Jmjd1c, Nrep, |
| | | | | | | Rbm39, Rlim, Rsrc1, Sat1, Slc3a2, |
| | | | | | | Sltm, Sobp, Tfdp2, Tspan17, |
| | | | | | | Txnrd1, Zfr |
| c3 | 2 | down | TGCTTTG MIR330 | 0.000 | 0.002 | Abhd2, Atp2b1, B4galt1, Bcor, |
| | | | | | | Btg1, Dicer1, Eif4e, Fchsd2, Fgfr1, |
| | | | | | | Hivep2, Id2, Kcna6, Kcnip2, |
| | | | | | | Luzp1, Mrfap1, Rnd3, Runx3, |
| | | | | | | Sec62, Sh3gl2, Shox2, Skil, |
| | | | | | | Smad7, Tiparp, Tpm3, Tra2b, |
| | | | | | | Trps1, Znrf2 |
| c3 | 2 | down | TTTGCAC MIR19A MIR19B | 0.000 | 0.025 | Abr, Actn1, Arfip1, Asap2, Atxn1, |
| | | | | | | Bambi, Btg1, Calm1, Cbfb, |
| | | | | | | Ccdc88a, Ccnl1, Cpeb4, Dicer1, |
| | | | | | | Ell2, F3, Fam114a1, Klf10, Klf13, |
| | | | | | | Lbh, Lrrk1, Npas2, Nr4a2, Nrp2, |
| | | | | | | Prrc2c, Reep3, Rnf2, Runx3, Sdc1, |
| | | | | | | Sept7, Smoc1, Socs1, Socs3, |
| | | | | | | Socs5, Tgfbr2, Tln2, Tnip1, Trps1, |
| | | | | | | Ubl3, Vgll4 |
| c3 | 2 | down | TTTGTAG MIR520D | 0.000 | 0.015 | Arl4a, Atp2b1, Atxn1, Bhlhe41, |
| | | | | | | Bzw1, Cpeb4, Ddit4l, Eif4g2, |
| | | | | | | H3f3b, Hes1, Hnrnpdl, Ier5, |
| | | | | | | Jmjd1c, Kcnk1, Map3k2, Pcnp, |
| | | | | | | Ppp2ca, Pum1, Rlim, Scd2, Sdc2, |
| | | | | | | Sec62, Smoc1, Socs5, Spats2l, |
| | | | | | | Spin1, Tiparp, Tmem47, Trps1, |
| | | | | | | Vgll4, Zfp385b |
| c3 | 2 | down | YTAATTAA LHX3 01 | 0.000 | 0.016 | Adamts10, Appl2, Bhlhe41, |
| | | | | | | Dixdc1, Ier3, Inppl1, Kat6b, Ldb2, |
| | | | | | | Nrep, Pitpnc1, Rora, Shox2, Sobp, |
| | | | | | | Sspn, Tfdp2, Zbtb20 |
| c3 | 2 | down | YY1 02 | 0.001 | 0.030 | Abce1, Ap4s1, Cnnm1, Ext1, |
| | | | | | | H1f0, Jtb, Mgat4b, Ppp2ca, Prrc2c, |
| | | | | | | Pum1, Rab1a, Rala, Rbm39, Sept7, |
| | | | | | | Tra2b, Ubl3, Wsb1 |
| c3 | 2 | down | YYCATTCAWW UNKNOWN | 0.001 | 0.029 | Atf3, Calm1, Cask, Ddx5, Eif4e, |
| | | | (SEQ ID NO; 12) | | | Fosb, Gclc, Id1, Lgals3, Nfia, |
| | | | | | | P4ha1, Pde4b, Ppa1, Smap2, Tob1, |
| | | | | | | Tpm3, Ube2n |
| c3 | 2 | up | EVI1 06 | 0.003 | 0.010 | Cited2, Il17b |
| c3 | 3 | down | CAC BINDING PROTEIN Q6 | 0.000 | 0.032 | Arhgap44, Atf3, Hif1a, Igfbp5, |
| | | | | | | Krtcap2, Nfix, Pim1, Ptms, Tppp3, |
| | | | | | | Ywhaq |
| c3 | 3 | down | FOXJ2 02 | 0.000 | 0.050 | Ackr3, Atf3, Atp2b4, Dnajb9, Id1, |
| | | | | | | Igfbp7, Klf3, Nfix, Pafah1b1, |
| | | | | | | Sobp, Srsf7, Tob1 |
| c3 | 3 | down | TGGNNNNNNKCCAR | 0.000 | 0.040 | Anxa3, Bhlhe40, Coa3, Col6a3, |
| | | | UNKNOWN (SEQ ID NO: 13) | | | Cryab, Csmp1, Dab2ip, Dennd5a, |
| | | | | | | Dusp14, Fads3, Fxyd1, Hbegf, |
| | | | | | | Hdlbp, Igfbp5, Kmt2e, Krtcap2, |
| | | | | | | Nfatc1, Nfkbia, Pcolce, Plat, |
| | | | | | | Sh2d4a, Smad7, Tppp3, Vasn |
| c3 | 3 | down | TTCNRGNNNNTTC HSF Q6 | 0.000 | 0.029 | Abhd2, BC030336, Cryab, Dnaja1, |
| | | | (SEQ ID NO: 8) | | | Dpysl2, Fgf10, Fosb, Hspa4l, |
| | | | | | | Hspa8, Pafah1b1, Pmp22, |
| | | | | | | Serpinh1, Stx4a, Tnxb |
| c3 | 3 | down | ZIC1 01 | 0.000 | 0.039 | Atp2b4, Atp6v0a4, Cggbp1, Cilp2, |
| | | | | | | Cryab, Dynll1, Fgf10, Lmna, |
| | | | | | | Ndufa412, Ostc, Pmp22, S100a4 |
| c3 | 4 | up | GCGCCTT MIR525 MIR524 | 0.079 | 0.037 | Ggnbp2 |
| c3 | 5 | down | ACTACCT MIR196A MIR196B | 0.003 | 0.034 | Cdv3, Col1a1, Col1a2, Col3a1, |
| | | | | | | Plpp6 |
| c3 | 5 | down | AP2GAMMA 01 | 0.000 | 0.005 | Atf5, Calm1, Cdon, Cxxc5, |
| | | | | | | Gprc5c, Hoxc10, Igfbp7, Klf10, |
| | | | | | | Map1b, Nr3c1, P4ha1, Ppm1e, |
| | | | | | | Smad7 |
| c3 | 5 | down | RFX1 02 | 0.000 | 0.046 | Adk, Crip2, Cxxc5, Fgf9, Plec, |
| | | | | | | Rreb1, Sparc, Ywhaq |
| c3 | 5 | down | ZIC1 01 | 0.000 | 0.040 | Ccdc88a, Cilp2, Cxxc5, Dynll1, |
| | | | | | | Lmna, Map1b, Ndufa4l2, Ostc, |
| | | | | | | Pmp22, Rora, S100a4 |
| c3 | 6 | down | AAAYWAACM HFH4 01 | 0.005 | 0.034 | Ankrd11, Cyr61, Dusp1, Fosb, |
| | | | | | | Hoxb4, Id2, Id4, Klf7, Mcc, Mgll, |
| | | | | | | Palmd, Pdcd4, Plpp3, Ppp1cb, |
| | | | | | | Sgk1, Smarca2, Tbx3, Tspan13 |
| c3 | 6 | down | AACATTC MIR4093P | 0.030 | 0.049 | Alpl, Cacul1, Cited2, Clock, Edn1, |
| | | | | | | Fosb, Foxp1, Jun, Tpm3 |
| c3 | 6 | down | AATGTGA MIR23A MIR23B | 0.008 | 0.047 | Arf6, Azin1, Dnm3, Fnbp1l, Fosb, |
| | | | | | | Gpbp1, Hoxb4, Irf1, Irf2, Kitl, |
| | | | | | | Mat2a, Nuak1, Pdia6, Phactr2, |
| | | | | | | Plcxd3, Ppp1cb, Sgk1, Tgfbr2, |
| | | | | | | Tpm3 |
| c3 | 6 | down | ACATTCC MIR1 MIR206 | 0.000 | 0.019 | Azin1, Cdk9, Cited2, Ddx5, Edn1, |
| | | | | | | Efnb2, Eif4e, Fosb, Foxp1, Hoxb4, |
| | | | | | | Jund, Klf13, Map4k3, Nrp1, |
| | | | | | | Timp3, Tpm3, Tra2b, Zfp36l1 |
| c3 | 6 | down | ACAWNRNSRCGG UNKNOWN | 0.014 | 0.034 | Ckb, Ddx5, Fkbp3, Nr4a1, Pum1, |
| | | | (SEQ ID NO: 14) | | | Sfpq, Tra2b |
| c3 | 6 | down | ACTGTAG MIR139 | 0.000 | 0.005 | Atrx, Azin1, Cdk19, Fos, Foxp1, |
| | | | | | | Jun, Mat2a, Ube2f |
| c3 | 6 | down | AHRARNT 01 | 0.000 | 0.000 | Dll4, Hes1, Hoxa5, Id4, Jag1, |
| | | | | | | Maea, Nr3c1, Nr4a1, Pcdh17, |
| | | | | | | Pdgfb, Rarg, Sgk1, Srrm2, Zbtb20 |
| c3 | 6 | down | AP2GAMMA 01 | 0.012 | 0.041 | Cdk9, Dll4, Gpbp1, Ier5l, Jund, |
| | | | | | | Kitl, Klf13, Map3k11, Mecom, |
| | | | | | | Nr3c1, Pdgfb, Pdlim2, Prdm16, |
| | | | | | | Rarg, Zfp687 |
| c3 | 6 | down | AP4 Q5 | 0.000 | 0.031 | Anp32a, Azin1, Btg1, Cited2, Dll4, |
| | | | | | | Dnaja1, Filip1, Id3, Irf2bpl, Klf13, |
| | | | | | | Pdgfb, Prex2, Slc26a10, Smarca2, |
| | | | | | | Tgfbr2, Tpm3 |
| c3 | 6 | down | AREB6 04 | 0.007 | 0.031 | Apold1, Arf6, Col8a1, Edn1, |
| | | | | | | Efnb2, Fbln5, Fchsd2, Fgf13, Junb, |
| | | | | | | Klf7, Mapk3, Nedd9, Pdcd4, |
| | | | | | | Pik3r3, Smarca2, Son, Srsf2, |
| | | | | | | Thap3 |
| c3 | 6 | down | ATAAGCT MIR21 | 0.020 | 0.042 | Cbx4, Epha4, Jag1, Pcdh17, |
| | | | | | | Pdcd4, Rhob, Ssfa2, Tgfbr2, |
| | | | | | | Timp3 |
| c3 | 6 | down | ATACTGT MIR144 | 0.007 | 0.026 | Arid5b, Cav2, Cbx1, Cbx4, |
| | | | | | | Ccdc88a, Cdk19, Fosb, Lifr, |
| | | | | | | Map7d1, Msx1, Nedd4, Nr3c1, |
| | | | | | | Pnrc1, Ppp1r16b, Pum1, Son |
| c3 | 6 | down | ATCMNTCCGY UNKNOWN | 0.014 | 0.047 | Atf4, Calm1, Ccdc88a, Cfap20, |
| | | | (SEQ ID NO: 15) | | | Srsf7, Ubc |
| c3 | 6 | down | ATF1 Q6 | 0.000 | 0.013 | Cd2ap, Ddit3, Fos, Fosb, Foxp1, |
| | | | | | | Id1, Irf2bpl, Jund, Mbnl2, Pdzd8, |
| | | | | | | Thra, Yeats2 |
| c3 | 6 | down | ATF3 Q6 | 0.000 | 0.001 | Atf3, Cd2ap, Cdkn1a, Dusp1, |
| | | | | | | Fosb, Gpbp1, Irf2bpl, Jun, Jund, |
| | | | | | | Klf13, Mbnl2, Pnrc1, Ppp1r15a, |
| | | | | | | Rfx1, Zfp36l1 |
| c3 | 6 | down | ATF4 Q2 | 0.000 | 0.005 | Dusp1, Fos, Fosb, Id1, Irf2bpl, |
| | | | | | | Itm2b, Jag1, Klf13, Mbnl2, Pnrc1, |
| | | | | | | Ppp1r10, Ppp1r15a, S1pr2, Sgk1, |
| | | | | | | Zbtb20 |
| c3 | 6 | down | ATF6 01 | 0.002 | 0.003 | Brd2, Dhx40, Egr1, Gpbp1, Irx3, |
| | | | | | | Jund, Klf13, Nr4a1, Ppp1r10, |
| | | | | | | Syt11, Tspan13 |
| c3 | 6 | down | ATF B | 0.003 | 0.042 | Cd2ap, Dctn1, Dusp1, Fos, Fosb, |
| | | | | | | Gpbp1, Id1, Irf2bpl, Jund, Mbnl2, |
| | | | | | | Pnrc1, Ppp1r15a |
| c3 | 6 | down | ATGTAGC MIR221 MIR222 | 0.024 | 0.035 | Cdk19, Cdkn1c, Fermt2, Fos, Irf2, |
| | | | | | | Klc1, Lifr, Midn, Sncb, Timp3 |
| c3 | 6 | down | ATGTTAA MIR302C | 0.012 | 0.049 | Azin1, Cdk19, Foxp1, H3f3b, |
| | | | | | | Hormad1, Lifr, Phactr2, Sipa1l2, |
| | | | | | | Son, Srsf2, Tob1, Ubb, Zfp36l1 |
| c3 | 6 | down | BRN2 01 | 0.000 | 0.015 | Arf6, Clock, Fgf13, Fos, H3f3b, |
| | | | | | | Mbnl2, Mecom, Mgll, Ppp1r16b, |
| | | | | | | Sat1, Stmn2, Tob1, Tspan13 |
| c3 | 6 | down | CAGCAGG MIR370 | 0.020 | 0.048 | Ccdc85b, Cit, Ctbp2, Fosb, Rgs3, |
| | | | | | | Tap2, Tgfbr2, Zc2hc1c |
| c3 | 6 | down | CAGCTTT MIR320 | 0.008 | 0.045 | Arid5b, Atrx, Azin1, Gpbp1, |
| | | | | | | Hoxb4, Klf13, Mat2a, Mcl1, Mn1, |
| | | | | | | Nrp1, Ppp1r16b, Son, Sptssa, |
| | | | | | | Srsf7, Tpm3, Usp25 |
| c3 | 6 | down | CAGTATT MIR200B MIR200C | 0.003 | 0.042 | Atrx, Cbx4, Cited2, Dusp1, Efnb2, |
| | | | MIR429 | | | Fermt2, Id2, Ier5, Klf4, Lrp1, |
| | | | | | | Map4k3, Nr3c1, Pcmtd1, Plpp3, |
| | | | | | | Pls3, Ppp1r10, Slc6a6, Srsf2, |
| | | | | | | Tob1, Usp25 |
| c3 | 6 | down | CCAATNNSNNNGCG | 0.000 | 0.014 | Chac1, Dnajb9, Hes1, Lman1, |
| | | | UNKNOWN (SEQ ID NO: 16) | | | Nck1, Pnrc1, Ppp1r10 |
| c3 | 6 | down | CCAWNWWNNNGGC | 0.004 | 0.030 | Atrx, Azin1, Fermt2, Hoxc6, Irf9, |
| | | | UNKNOWN (SEQ ID NO: 17) | | | Sat1, Ube2k |
| c3 | 6 | down | CCAWWNAAGG SRF Q4 (SEQ | 0.018 | 0.031 | Atrx, Egr1, Fosb, Kctd15, Klf6, |
| | | | ID NO: 18) | | | Nr4a1 |
| c3 | 6 | down | CCAWYNNGAAR UNKNOWN | 0.019 | 0.042 | Arhgap5, Eif1, H3f3b, Id4, Jun, |
| | | | (SEQ ID NO: 19) | | | Mecom, Pradc1, Rhob, Scube3 |
| c3 | 6 | down | CCTGTGA MIR513 | 0.007 | 0.026 | Btg1, Dnm3, Ier5, Nebl, Ppp1r16b, |
| | | | | | | Srrm2, Ube2k |
| c3 | 6 | down | CDPCR1 01 | 0.007 | 0.020 | Egr1, Hoxb4, Kcns1, Mn1, Son, |
| | | | | | | Stmn2, Tbx3, Thra |
| c3 | 6 | down | CDP 01 | 0.013 | 0.020 | Egr1, Fgf13, Mn1, Stmn2 |
| c3 | 6 | down | CEBPB 02 | 0.000 | 0.004 | Arf6, Cbx4, Dusp1, Foxp1, H3f3b, |
| | | | | | | Hoxa5, Id3, Nfkbia, Nfkbiz, Pdgfb, |
| | | | | | | Ppp1cb, Rhob, Smarca2, Sulf1, |
| | | | | | | Thra, Tob1, Zbtb20 |
| c3 | 6 | down | CEBPDELTA Q6 | 0.002 | 0.023 | Ace, Ankrd11, Asb18, Atf3, Ccnl1, |
| | | | | | | Cited2, Fos, Fosb, Foxp1, Jun, |
| | | | | | | Mbnl2, Nr4a1, Nsd1, Ppp1r10, |
| | | | | | | Syt11, Thra, Timp3 |
| c3 | 6 | down | CEBP Q2 | 0.000 | 0.002 | Arf6, Arhgef6, Cbx4, Cdkn1c, |
| | | | | | | Clock, Ddit3, Foxp1, H3f3b, Id3, |
| | | | | | | Ier5l, Nfkbia, Nfkbiz, Osmr, |
| | | | | | | Pdgfb, Plcb1, Ppp1cb, Prdm16, |
| | | | | | | Rnf152, Smarca2, Srsf2, Thra, |
| | | | | | | Tm6sf1, Tob1, Zbtb20, Zfp36l1 |
| c3 | 6 | down | CEBP Q2 01 | 0.008 | 0.036 | Arf6, Cbx4, Cdkn1c, Cyr61, |
| | | | | | | Foxp1, H3f3b, Hoxa5, Id3, Jun, |
| | | | | | | Lrit3, Nfkbiz, Plcb1, Ppp1cb, |
| | | | | | | Rhob, Thra, Tob1, Zbtb20, Zfp36l1 |
| c3 | 6 | down | CEBP Q3 | 0.000 | 0.000 | Anp32a, Brd2, Cbx4, Cyr61, |
| | | | | | | Ddit3, Hoxa5, Id3, Jun, Junb, Kitl, |
| | | | | | | Mn1, Nfkbiz, Pdgfb, Rhob, Rora, |
| | | | | | | Syne2, Tgfb2, Thap3, Thra, |
| | | | | | | Tnfsf10, Tob1, Zbtb20 |
| c3 | 6 | down | CMYB 01 | 0.010 | 0.040 | Arf6, Ash1l, Cfap20, Cited2, |
| | | | | | | Dnajb1, Hoxb4, Ier5, Jund, Mgll, |
| | | | | | | Mllt10, Pdgfb, Pik3r3, Tpm3, |
| | | | | | | Ube2k |
| c3 | 6 | down | CP2 01 | 0.002 | 0.024 | Arf6, Atf3, Dctn1, Dll4, Mapk3, |
| | | | | | | Nfkbiz, Rgs3, Scube3, Smarca2, |
| | | | | | | Socs1, Srsf7 |
| c3 | 6 | down | CP2 02 | 0.003 | 0.031 | Arhgap5, Dnajb1, H3f3b, Hspa1a, |
| | | | | | | Ier5l, Irx3, Junb, Mapk3, |
| | | | | | | Ppp1r16b, Scube3, Tgfbr2, Upk2 |
| c3 | 6 | down | CREBP1CJUN 01 | 0.000 | 0.016 | Cd2ap, Dctn1, Dusp1, Ebf1, Egr1, |
| | | | | | | Gpbp1, Id1, Irf2bpl, Jund, Mbnl2, |
| | | | | | | Pnrc1, Ppp1r15a, Syt11 |
| c3 | 6 | down | CREBP1 Q2 | 0.000 | 0.006 | Cd2ap, Dusp1, Egr1, Fgf13, Fos, |
| | | | | | | Fosb, Gpbp1, Id1, Id3, Irf2bpl, |
| | | | | | | Klf13, Pnrc1, Ppp1r10, Ppp1r15a, |
| | | | | | | Thra |
| c3 | 6 | down | CREB 01 | 0.002 | 0.022 | Atf3, Cd2ap, Dusp1, Fosb, Gpbp1, |
| | | | | | | Id1, Irf2bpl, Jund, Mbnl2, Pnrc1, |
| | | | | | | Ppp1r15a, Syt11, Zfp687 |
| c3 | 6 | down | CREB 02 | 0.004 | 0.038 | Cd2ap, Dctn1, Dusp1, Fosb, Id1, |
| | | | | | | Irf2bpl, Jund, Mbnl2, Ppp1r15a |
| c3 | 6 | down | CREB Q2 | 0.000 | 0.001 | Atf3, Cd2ap, Dusp1, Egr1, Fos, |
| | | | | | | Fosb, Gadd45b, Gpbp1, Id1, |
| | | | | | | Irf2bpl, Jag1, Klf13, Mbnl2, |
| | | | | | | Nr3c1, Pnrc1, Ppp1r15a, Pqlc1, |
| | | | | | | Zbtb20, Zfp687 |
| c3 | 6 | down | CREB Q2 01 | 0.000 | 0.001 | Cd2ap, Dusp1, Egr1, Fosb, Gpbp1, |
| | | | | | | Id1, Irf2bpl, Jun, Jund, Mbnl2, |
| | | | | | | Pnrc1, Ppp1r15a, Syt11, Tra2b, |
| | | | | | | Zfp687 |
| c3 | 6 | down | CREB Q3 | 0.002 | 0.019 | Arid4a, Dnm3, Dusp1, Ebf1, |
| | | | | | | Efhd1, Jag1, Jund, Map3k11, |
| | | | | | | Mbnl2, Syt11, Zfp687 |
| c3 | 6 | down | CREB Q4 | 0.000 | 0.004 | Atf3, Cd2ap, Dusp1, Fos, Fosb, |
| | | | | | | Gpbp1, Id1, Irf2bpl, Irx3, Jag1, |
| | | | | | | Klf13, Mbnl2, Pnrc1, Ppp1r15a, |
| | | | | | | Pqlc1, Psmb8, Zbtb20, Zfp687 |
| c3 | 6 | down | CREB Q4 01 | 0.000 | 0.034 | Cd2ap, Dctn1, Dusp1, Fosb, |
| | | | | | | Gpbp1, Id1, Irf2bpl, Jund, Mbnl2, |
| | | | | | | Ppp1r15a |
| c3 | 6 | down | CTAWWWATA RSRFC4 Q2 | 0.000 | 0.020 | Amd1, Atf3, Car7, Ckb, Col8a1, |
| | | | | | | Efhd1, Foxp1, Hes1, Hoxb4, |
| | | | | | | Hspb1, Jun, Mat2a, Nr4a1, Pik3r3, |
| | | | | | | Smarca2, Tob2, Tpm3, Tspan13 |
| c3 | 6 | down | CTTGTAT MIR381 | 0.002 | 0.015 | Azin1, Epha4, Klf4, Mbnl2, |
| | | | | | | Mecom, Nfkbia, Pnrc1, Sipa1l2, |
| | | | | | | Srrm1 |
| c3 | 6 | down | DR4 Q2 | 0.005 | 0.031 | Amd1, Ctbp2, Cx3cl1, Edn1, |
| | | | | | | Foxp1, Id2, Nfkbia, Ogn, Pdgfb, |
| | | | | | | Sgk1, Son, Srsf7, Zbtb20 |
| c3 | 6 | down | E2F1DP1RB 01 | 0.009 | 0.030 | Arid4a, Atrx, Id3, Ier5l, Klf4, |
| | | | | | | Mat2a, Srsf2, Srsf7, Zfp687 |
| c3 | 6 | down | E2F1 Q3 | 0.005 | 0.019 | Arid4a, Dolk, Fam46b, Id3, Ier5l, |
| | | | | | | Irx3, Srsf7, Syt11, Tbx3, Trim47, |
| | | | | | | Zfp687 |
| c3 | 6 | down | E2F1 Q4 01 | 0.013 | 0.049 | Arid4a, Atrx, Csrnp1, Dolk, Id3, |
| | | | | | | Ier5l, Mat2a, Pik3r3, Srsf2, Zfp687 |
| c3 | 6 | down | E2F 03 | 0.011 | 0.039 | Anp32a, Arid4a, Cdkn1a, Csrnp1, |
| | | | | | | Dll4, Fus, Id3, Ier5l, Mat2a, |
| | | | | | | Mecom, Rsrc2, Srsf2, Tbx3, |
| | | | | | | Zfp687 |
| c3 | 6 | down | E2F Q3 | 0.005 | 0.009 | Arid4a, Atrx, Dolk, Fam46b, Id3, |
| | | | | | | Ier5l, Klf4, Mat2a, Srsf2, Syt11, |
| | | | | | | Tbx3, Trim47 |
| c3 | 6 | down | E2F Q3 01 | 0.011 | 0.041 | Arid4a, Cited2, Csrnp1, Dolk, Id3, |
| | | | | | | Ier5l, Pik3r3, Srsf2, Tbx3, Trim47, |
| | | | | | | Zfp687 |
| c3 | 6 | down | E2F Q4 01 | 0.000 | 0.014 | Arid4a, Cited2, Csrnp1, Dolk, Id3, |
| | | | | | | Ier5l, Mat2a, Pik3r3, Rsrc2, Srsf2, |
| | | | | | | Syt11, Tbx3, Zfp687 |
| c3 | 6 | down | E2F Q6 01 | 0.002 | 0.011 | Arid4a, Cdkn1a, Cited2, Csrnp1, |
| | | | | | | Cyr61, Dll4, Dolk, Id3, Ier5l, |
| | | | | | | Mat2a, Sncb, Srsf2, Tbx3 |
| c3 | 6 | down | E47 01 | 0.000 | 0.012 | Aig1, Amd1, Car7, Cited2, Dll4, |
| | | | | | | Foxp1, Id1, Ier5l, Irf1, Nsd1, |
| | | | | | | Pcdh17, Pdgfb, Plcb1, Podxl, |
| | | | | | | Sh3glb2, Thra |
| c3 | 6 | down | E4F1 Q6 | 0.000 | 0.008 | Azin1, Cd2ap, Ckb, Cyr61, Dnm3, |
| | | | | | | Dusp1, Fosb, Gpbp1, Id1, Irf2bpl, |
| | | | | | | Nedd9, Nr3c1, Nr4a1, Pdzd8, |
| | | | | | | Ppp1r15a, S1pr2, Tspan13 |
| c3 | 6 | down | EGR1 01 | 0.000 | 0.003 | Ace, App, Brd2, Clstn1, Ddx5, |
| | | | | | | Dll4, Dynll1, Egr1, Fus, Hoxa5, |
| | | | | | | Irf2bpl, Kremen2, Mgll, Pcdh17, |
| | | | | | | Pdgfb, Rhob, Sertad1, Tbx3, Tra2b |
| c3 | 6 | down | EGR Q6 | 0.002 | 0.035 | Abi2, Ace, Brd2, Cbx4, Dll4, Egr1, |
| | | | | | | Fchsd2, Irf2bpl, Klf13, Mgll, |
| | | | | | | Nuak1, Pcdh17, Pdgfb, Pdzd8, |
| | | | | | | Ppp1r16b, Sertad1, Zfp36, Zfp36l1 |
| c3 | 6 | down | EVI1 04 | 0.014 | 0.049 | Ebf1, Edn1, H3f3b, Klf7, Lrp1, |
| | | | | | | Npnt, Plpp3, Rarg, Rhob, Tgfb2, |
| | | | | | | Tob1 |
| c3 | 6 | down | FAC1 01 | 0.007 | 0.017 | Cbx4, Efnb1, Fbln2, Filip1, Foxp1, |
| | | | | | | Gabarapl2, Hey 1, Hoxb4, Id4, |
| | | | | | | Junb, Klf11, Klf7, Pdcd4, Pik3r3, |
| | | | | | | Rfx1, Sat1, Sgk1, Smarca2, Tapbp |
| c3 | 6 | down | FOXD3 01 | 0.000 | 0.001 | Ankrd11, Arid4a, Cited2, Cyr61, |
| | | | | | | Id2, Ier3, Junb, Npnt, Nr4a1, Nrp1, |
| | | | | | | Pcdh17, Ppp1cb, Scube3, Sgk1, |
| | | | | | | Tgfb2, Tob1, Utrn |
| c3 | 6 | down | FOXJ2 01 | 0.000 | 0.006 | Cxcl1, Hoxc6, Id1, Id2, Ier3, Mgll, |
| | | | | | | Pcdh17, Ppp1cb, Prex2, Rora, |
| | | | | | | Runx2, Scube3, Tgfb2, Zbtb20 |
| c3 | 6 | down | FOXJ2 02 | 0.000 | 0.001 | Arf6, Atf3, Dnajb9, Edn1, Fchsd2, |
| | | | | | | Foxp1, Id1, Lifr, Mecom, Npnt, |
| | | | | | | Sar1b, Scube3, Srsf7, Tob1, Utrn |
| c3 | 6 | down | FOXO1 01 | 0.007 | 0.035 | Arid4a, Cdkn1c, Cyr61, Dusp1, |
| | | | | | | H3f3b, Hoxb4, Ier5l, Kdm6a, |
| | | | | | | Mgll, Pdcd4, Ppp1cb, Prex2, |
| | | | | | | Rbfox1, Scube3, Thra |
| c3 | 6 | down | FOXO1 02 | 0.000 | 0.003 | Arid4a, Cdkn1a, Cdkn1c, Clstn1, |
| | | | | | | Ddx5, Hlx, Hoxb4, Id2, Jag1, Lifr, |
| | | | | | | Rfx1, Tcf15, Thra, Tspan13 |
| c3 | 6 | down | FOXO3 01 | 0.000 | 0.002 | Arid4a, Cdkn1c, Cited2, Col8a1, |
| | | | | | | Hlx, Id2, Junb, Kitl, Mbnl2, Pdcd4, |
| | | | | | | Polr2l, Ppp1cb, Scube3, Srsf7 |
| c3 | 6 | down | FOXO4 01 | 0.003 | 0.018 | Cited2, Col8a1, Cyr61, Fchsd2, |
| | | | | | | H3f3b, Hoxb4, Id2, Mecom, |
| | | | | | | Nr3c1, Pdcd4, Polr2l, Thra, |
| | | | | | | Tspan13 |
| c3 | 6 | down | FOXO4 02 | 0.009 | 0.038 | Arid4a, Cdkn1c, Cited2, Col8a1, |
| | | | | | | Hlx, Hoxb4, Id2, Jag1, Nr3c1, |
| | | | | | | Pdcd4, Pdgfd, Polr2l, Scube3, |
| | | | | | | Tcf15, Thra |
| c3 | 6 | down | FOX Q2 | 0.000 | 0.003 | Cdkn1a, Fgf13, Junb, Mgll, Nebl, |
| | | | | | | Palmd, Pdcd4, Ppp1cb, Prex2, |
| | | | | | | Scube3, Slc20a1, Smarca2, |
| | | | | | | Tnfsf10, Tob1, Zbtb20 |
| c3 | 6 | down | FREAC2 01 | 0.002 | 0.011 | Arid4a, Cd2ap, Cdkn1a, Cdkn1c, |
| | | | | | | Cited2, Dusp1, Hoxb4, Id2, Mbnl2, |
| | | | | | | Mcc, Mecom, Plpp3, Rora, |
| | | | | | | Scube3, Tcf15, Thra, Tob1 |
| c3 | 6 | down | FREAC3 01 | 0.003 | 0.019 | Arid4a, Cdkn1a, Cdkn1c, Cited2, |
| | | | | | | Gpbp1, Id2, Mbnl2, Pou2f1, |
| | | | | | | Ppp1cb, Ppp1r10, Tcf15, Thap3, |
| | | | | | | Thra, Vps45, Zbtb20 |
| c3 | 6 | down | FREAC4 01 | 0.005 | 0.006 | Cdkn1a, Cited2, Dusp1, Hoxc6, |
| | | | | | | Id2, Ier5, Mast4, Mbnl2, Mcc, |
| | | | | | | Nr4a1, Pls3, Pnrc1, Rarg |
| c3 | 6 | down | FREAC7 01 | 0.002 | 0.019 | Alpl, Cxcl1, Hoxb4, Ier3, Junb, |
| | | | | | | Mgll, Pou2f1, Ppp1cb, Prex2, |
| | | | | | | Rhob, Scube3, Tgfb2, Zbtb20 |
| c3 | 6 | down | GAGCTGG MIR337 | 0.000 | 0.016 | Brd2, Btg2, Cacul1, Efnb1, Fos, |
| | | | | | | Ier5l, Junb, Klf11, Klf13, Mat2a, |
| | | | | | | Thra |
| c3 | 6 | down | GATA1 04 | 0.007 | 0.031 | Aif1l, Gadd45g, Hes1, Id3, Jun, |
| | | | | | | Lmo2, Map4k3, Mbnl2, Mecom, |
| | | | | | | Nr3c1, Pik3r3, Ptprr, S1pr2, |
| | | | | | | Scube3, Zbtb20 |
| c3 | 6 | down | GATA1 05 | 0.002 | 0.012 | Dis3l, Fermt2, Filip1, Gpbp1, |
| | | | | | | Hes1, Hey 1, Id3, Jun, Lmo2, |
| | | | | | | Mecom, Msx1, Nr3c1, Pik3r3, |
| | | | | | | Ptprr, Syne2, Tgfb2, Zbtb20 |
| c3 | 6 | down | GATTGGY NFY Q6 01 | 0.000 | 0.043 | Arid4a, Atf4, B2m, Ccdc85b, Cit, |
| | | | | | | Ddit3, Dlg3, Dnajb1, Ebf1, Fkbp3, |
| | | | | | | Fosb, H3f3b, Hspa1a, Hspa1b, |
| | | | | | | Hspa5, Id4, Ier5, Ier5l, Irx3, Junb, |
| | | | | | | Mbnl2, Mn1, Msx1, Mycbp2, |
| | | | | | | Pcdh17, Pdcd4, Pik3r3, Pradc1, |
| | | | | | | Rgs3, Rhob, Sertad1, Sfr1, Son, |
| | | | | | | Srsf2, Stmn2, Tbx3, Tgfbr2, |
| | | | | | | Tspan13, Uaca, Ubb, Zbtb20 |
| c3 | 6 | down | GCACTTT MIR175P MIR20A | 0.000 | 0.031 | App, Arid4a, Cdkn1a, Clock, |
| | | | MIR106A MIR106B MIR20B | | | Ddx5, Dnajb9, Efnb1, Epas1, |
| | | | MIR519D | | | Epha4, Fam13c, Fgd5, Fnbp1l, |
| | | | | | | Irf1, Klf11, Lima1, Map3k11, |
| | | | | | | Mcl1, Midn, Plcb1, Rsrc2, Sar1b, |
| | | | | | | Srsf2, Ssfa2, Tbx3, Tgfbr2, Thra, |
| | | | | | | Tnfsf12, Topors |
| c3 | 6 | down | GCATTTG MIR105 | 0.012 | 0.044 | Epha4, Gabarapl2, H3f3b, Itm2b, |
| | | | | | | Son, Sptssa, Tpm3, Tra2b, Tulp4 |
| c3 | 6 | down | GCCATNTTG YY1 Q6 | 0.000 | 0.015 | Arid4a, Atf4, Atrx, Dolk, Foxp1, |
| | | | | | | Fus, Gpbp1, Hspa8, Lima1, Maea, |
| | | | | | | Map4k3, Mat2a, Nfe2l1, Pum1, |
| | | | | | | Rsrc2, Srsf2, Srsf7, Syne2, Syt11, |
| | | | | | | Tra2b, Ubc |
| c3 | 6 | down | GCGNNANTTCC UNKNOWN | 0.018 | 0.032 | Amd1, Cdkn1a, Dnaja1, |
| | | | (SEQ ID NO: 20) | | | Gabarapl2, Qpctl |
| c3 | 6 | down | GGATTA PITX2 Q2 | 0.000 | 0.007 | Acvrl1, Arf6, Btg1, Cbx4, Cd2ap, |
| | | | | | | Clock, Ctbp2, Dll4, Gadd45b, |
| | | | | | | Gpbp1, Hoxb4, Irx3, Jund, Kitl, |
| | | | | | | Lifr, Lima1, Mbnl2, Msx1, Nebl, |
| | | | | | | Pou2f1, Ppp1r10, Ppp1r16b, Skil, |
| | | | | | | Smarca2, Tob2, Ubxn4, Utrn, |
| | | | | | | Zfp36l1 |
| c3 | 6 | down | GGCACAT MIR455 | 0.017 | 0.049 | Edn1, Plcxd3, Socs3 |
| c3 | 6 | down | GGCAGTG MIR3243P | 0.032 | 0.042 | Atrx, Cit, Ckb, Ddx5, Efnb2, |
| | | | | | | Filip1, Gpatch8, Nrp1, Slc20a1, |
| | | | | | | Tob2 |
| c3 | 6 | down | GGCNKCCATNK UNKNOWN | 0.004 | 0.013 | Arid4a, Brd2, Nsd1, Pdlim2, |
| | | | (SEQ ID NO: 21) | | | Pum1, Rgs3, Sar1b, Tob2 |
| c3 | 6 | down | GTACAGG MIR486 | 0.013 | 0.035 | Arhgap5, Dynll1, Fgf13, H3f3b, |
| | | | | | | Id4, Tob1 |
| c3 | 6 | down | GTATTAT MIR3693P | 0.002 | 0.025 | Arid4a, Arl2bp, Cbx4, Cd2ap, |
| | | | | | | Cdk19, Efnb2, Eps8l2, Fnbp1l, |
| | | | | | | Hes1, Lrp1, Mbnl2, Nuak1, Pnrc1, |
| | | | | | | Vegfc |
| c3 | 6 | down | GTCNYYATGR UNKNOWN | 0.002 | 0.027 | Azin1, Id1, Jun, Rarg, Scube3, |
| | | | (SEQ ID NO: 22) | | | Skil, Wdr54 |
| c3 | 6 | down | GTGACGY E4F1 Q6 | 0.000 | 0.010 | Amd1, Cd2ap, Cdkn1a, Cyr61, |
| | | | | | | Dnaja1, Dusp1, Egr1, Fosb, |
| | | | | | | Gpbp1, Id1, Irf2bpl, Junb, Jund, |
| | | | | | | Mllt10, Nr4a1, Pdzd8, Pnrc1, |
| | | | | | | Ppp1r10, Ppp1r15a, Pqlc1, Psmb8, |
| | | | | | | S1pr2, Tgfb2, Thra, Tnfsf12, |
| | | | | | | Tra2b, Ubb, Zbtb20 |
| c3 | 6 | down | GTGACTT MIR224 | 0.000 | 0.002 | Azin1, Cdk9, Cit, Fosb, H3f3b, |
| | | | | | | Hoxa5, Id3, Itm2b, Mbnl2, Nr4a1, |
| | | | | | | Nuak1, Plpp3, Rere, Runx2, Tra2b, |
| | | | | | | Zdhhc20 |
| c3 | 6 | down | GTGCAAT MIR25 MIR32 MIR92 | 0.000 | 0.005 | Atrx, Btg2, Cd2ap, Cdkn1c, |
| | | | MIR363 MIR367 | | | Dnajb9, Klf2, Klf4, Mcl1, |
| | | | | | | Mycbp2, Pik3r3, Rfx1, Rgs3, |
| | | | | | | Scube3, Ssfa2, Tob1, Tob2, Tulp4 |
| c3 | 6 | down | HFH3 01 | 0.000 | 0.000 | Ccdc85b, Cyr61, Dusp1, Id1, Id2, |
| | | | | | | Klf7, Mcc, Palmd, Pcdh17, |
| | | | | | | Ppp1cb, Runx2, Scube3, Sgk1, |
| | | | | | | Smarca2, Tob1 |
| c3 | 6 | down | HFH4 01 | 0.000 | 0.003 | Cited2, Eif4e, H3f3b, Id1, Id2, |
| | | | | | | Mgll, Pcdh17, Ppp1cb, Scube3, |
| | | | | | | Sgk1, Tspan13 |
| c3 | 6 | down | HIF1 Q5 | 0.000 | 0.008 | Ak3, Cited2, Dll4, Fchsd2, Fkbp3, |
| | | | | | | Gadd45b, Klf11, Pdgfb, Ppt2, |
| | | | | | | Prdx4, Rora, Slc26a10 |
| c3 | 6 | down | HMX1 01 | 0.005 | 0.040 | Id1, Junb, Kremen2, Slc20a1 |
| c3 | 6 | down | HNF3 ALPHA Q6 | 0.000 | 0.009 | Cdkn1a, Cyr61, Fgf13, Foxp1, Id1, |
| | | | | | | Mcc, Mgll, Nebl, Palmd, Pcdh17, |
| | | | | | | Pdcd4, Ppp1cb, Sgk1, Smarca2, |
| | | | | | | Tob1 |
| c3 | 6 | down | HNF3B 01 | 0.000 | 0.001 | Cited2, Cyr61, Dusp1, Id1, Id2, |
| | | | | | | Ier3, Pdcd4, Plcb1, Ppp1cb, |
| | | | | | | Ppp1r10, Rarg, Scube3, Sprr4, |
| | | | | | | Tob1 |
| c3 | 6 | down | HNF3 Q6 | 0.000 | 0.001 | Cdkn1a, Cyr61, Fgf13, Fosb, Id1, |
| | | | | | | Klf7, Nebl, Nr4a1, Palmd, Pcdh17, |
| | | | | | | Pdcd4, Ppp1cb, Scube3, Sgk1, |
| | | | | | | Smarca2, Tob1 |
| c3 | 6 | down | HSF Q6 | 0.014 | 0.048 | Atf3, Csrnp1, Cyr61, Dnajb1, |
| | | | | | | Edn1, Gpbp1, Hspa1a, Hspa1b, |
| | | | | | | Msx1, Thap3, Ubb, Ube2k |
| c3 | 6 | down | HTF 01 | 0.000 | 0.009 | Dnajb4, Dnm3, Mn1, Sgk1, Srsf2, |
| | | | | | | Syt11, Tspan13 |
| c3 | 6 | down | IK1 01 | 0.000 | 0.002 | Apold1, Arhgap5, Cited2, Ddx5, |
| | | | | | | Dll4, Filip1, Fnbp1l, Foxp1, |
| | | | | | | Gadd45b, Hoxb4, Irf2bpl, Itm2b, |
| | | | | | | Jun, Lifr, Lrp1, Msx1, Nsd1, |
| | | | | | | Pcdh17, Plpp3, Ppp1r10, Prex2, |
| | | | | | | Sema3g, Srsf7 |
| c3 | 6 | down | IK3 01 | 0.000 | 0.003 | Bcam, Cdk9, Cyr61, Egr1, Fos, |
| | | | | | | Hoxb4, Jag1, Lifr, Mbnl2, Msx1, |
| | | | | | | Nfkbia, Nsd1, Plpp3, Ppp1cb, |
| | | | | | | Slc50a1, Timp3, Ubxn4, Usp25, |
| | | | | | | Zeb1 |
| c3 | 6 | down | IRF1 01 | 0.000 | 0.009 | Arhgap5, Arhgef6, Atf3, B2m, |
| | | | | | | Bst2, Ddit3, Epas1, Fam13c, Hlx, |
| | | | | | | Irf9, Isg15, Map3k11, Prdm16, |
| | | | | | | Psmb10, Psmb8, Psmb9, Ptprr, |
| | | | | | | Sat1, Syne2, Tapbp, Xaf1 |
| c3 | 6 | down | IRF1 Q6 | 0.000 | 0.002 | Arhgap5, Ebf1, Fam96b, Hoxb4, |
| | | | | | | Klf6, Lysmd2, Map3k11, Msx1, |
| | | | | | | Prkd2, Psmb10, Sgk1, Slc12a7, |
| | | | | | | Snai1, Syne2, Tapbp, Tcf15, |
| | | | | | | Zbtb20 |
| c3 | 6 | down | IRF2 01 | 0.005 | 0.006 | Arhgap5, Arhgef6, Ash1l, Atf3, |
| | | | | | | B2m, Bst2, Fam13c, Map3k11, |
| | | | | | | Msx1, Prdm16, Psmb8, Psmb9, |
| | | | | | | Syne2, Tapbp, Xaf1 |
| c3 | 6 | down | IRF7 01 | 0.000 | 0.001 | Arf6, Bst2, Cbx4, Dll4, Fos, Grrp1, |
| | | | | | | Irf2, Isg15, Lgals3bp, Msx1, Nrp1, |
| | | | | | | Ppp1r10, Prdm16, Psmb9, Sat1, |
| | | | | | | Scube3, Secisbp2l, Slc12a7, Socs1, |
| | | | | | | Tapbp, Tcf15, Xaf1, Zfp36l1 |
| c3 | 6 | down | IRF Q6 | 0.000 | 0.001 | Arhgap5, Arhgef6, B2m, Bst2, |
| | | | | | | Cbx4, Hoxb4, Irf2, Irf9, Isg15, |
| | | | | | | Lmo2, Map3k11, Msx1, Nrp1, |
| | | | | | | Prdm16, Psmb10, Psmb8, Rarg, |
| | | | | | | Sat1, Socs1, Syne2, Tapbp, Tcf15 |
| c3 | 6 | down | ISRE 01 | 0.000 | 0.016 | Arhgef6, B2m, Bst2, Cbx4, Chka, |
| | | | | | | Irf2, Irf9, Isg15, Map3k11, Msx1, |
| | | | | | | Npnt, Pik3r3, Prdm16, Slc12a7, |
| | | | | | | Tapbp, Tcf15, Xaf1 |
| c3 | 6 | down | LHX3 01 | 0.000 | 0.001 | Dll4, Fbln2, H3f3b, Id3, Junb, |
| | | | | | | Kcns1, Klf7, Lysmd2, Nebl, Nrp1, |
| | | | | | | Pcmtd1, Pik3r3, Srsf7, Tob1, |
| | | | | | | Zbtb20 |
| c3 | 6 | down | LMO2COM 01 | 0.000 | 0.019 | Amd1, Anp32a, Car7, Cited2, |
| | | | | | | Dctn1, Id1, Irf1, Jag1, Pdgfb, |
| | | | | | | Ppp1r16b, Rarg, Stmn2, Tspan13 |
| c3 | 6 | down | MEF2 01 | 0.016 | 0.032 | Aggf1, Anp32a, Car7, Dll4, Fos, |
| | | | | | | Hoxb4, Jun, Mbnl2, Nedd4, |
| | | | | | | Smarca2 |
| c3 | 6 | down | MEF2 02 | 0.010 | 0.042 | Atf3, Car7, Col8a1, Dll4, Foxp1, |
| | | | | | | Hoxb4, Jun, Npnt, Nr4a1, Pcmtd1, |
| | | | | | | Rgs3, Smarca2 |
| c3 | 6 | down | MEF2 03 | 0.002 | 0.012 | Atf3, Car7, Col8a1, Efhd1, Fos, |
| | | | | | | Foxp1, Hlx, Jun, Mbnl2, Nr4a1, |
| | | | | | | Pik3r3, Rhob, Smarca2 |
| c3 | 6 | down | MEF2 Q6 01 | 0.007 | 0.040 | Arl6ip5, Car7, Col8a1, Ctbp2, |
| | | | | | | Dll4, Fgf13, Filip1, Foxp1, |
| | | | | | | Gabarapl2, Jun, Mbnl2, Ppp1r16b, |
| | | | | | | Rhob, Scube3, Smarca2 |
| c3 | 6 | down | MEIS1 01 | 0.007 | 0.037 | Ash1l, Cbx4, Copz2, Ddit3, Fgf13, |
| | | | | | | Fkbp3, Fos, Hlx, Klf7, Nrp1, |
| | | | | | | Pik3r3, Scube3, Snai1, Thra |
| c3 | 6 | down | NFKAPPAB65 01 | 0.008 | 0.022 | Ash1l, Clock, Ebf1, Gadd45b, |
| | | | | | | Gpbp1, Ier3, Ier5, Map3k11, |
| | | | | | | Msx1, Nfkbia, S1pr2, Trim47, |
| | | | | | | Uaca |
| c3 | 6 | down | NFKAPPAB 01 | 0.012 | 0.031 | Arhgap5, Ash1l, Azin1, Cxcl1, |
| | | | | | | Gadd45b, Gpbp1, Ier5, Irf1, Msx1, |
| | | | | | | S1pr2, Trim47, Uaca, Vcam1, |
| | | | | | | Zfp36l2 |
| c3 | 6 | down | NFKB C | 0.000 | 0.000 | Anp32a, Arhgap5, Ash1l, Dnaja1, |
| | | | | | | Ebf1, Fos, Gadd45b, Gpbp1, Id2, |
| | | | | | | Ier5, Irf1, Msx1, Nfkbia, S1pr2, |
| | | | | | | Trim47, Uaca, Vcam1 |
| c3 | 6 | down | NFKB Q6 | 0.000 | 0.019 | Ash1l, Cxcl1, Cyr61, Ebf1, Ier5, |
| | | | | | | Irf2bpl, Lrp1, Nfkbia, Nsd1, Rgs3, |
| | | | | | | Slc50a1, Tcf15, Trim47, Uaca, |
| | | | | | | Vcam1 |
| c3 | 6 | down | NFKB Q6 01 | 0.003 | 0.027 | Ash1l, Ebf1, Gadd45b, Gpbp1, |
| | | | | | | Ier5, Map3k11, Msx1, Nfkbia, |
| | | | | | | Nr3c1, S1pr2, Trim47, Uaca, |
| | | | | | | Vcam1 |
| c3 | 6 | down | NFY 01 | 0.002 | 0.013 | Cit, Dlg3, H3f3b, Hes1, Hspa1a, |
| | | | | | | Ier5, Junb, Klf11, Pik3r3, Pnrc1, |
| | | | | | | Rgs3, Rhob, Slc20a1, Srsf2, |
| | | | | | | Tspan13, Ubb, Zfp36l2 |
| c3 | 6 | down | NKX61 01 | 0.000 | 0.005 | Clock, Hoxc6, Mbnl2, Nsd1, |
| | | | | | | Pcmtd1, Plcb1, Rnf152, Sat1, |
| | | | | | | Slc20a1, Sox18, Tob1, Zbtb20, |
| | | | | | | Zfp36l1 |
| c3 | 6 | down | OCT1 01 | 0.002 | 0.017 | Cited2, Cyr61, Foxp1, H3f3b, |
| | | | | | | Hoxa5, Id3, Id4, Ing1, Mcc, |
| | | | | | | Pou2f1, Rhob, Skil, Thra, Tspan13, |
| | | | | | | Zbtb20 |
| c3 | 6 | down | OCT1 05 | 0.000 | 0.019 | Ebf1, Edn1, Fos, Gadd45g, Hoxa5, |
| | | | | | | Ing1, Nedd9, Pou2f1, Rgs3, Rhob, |
| | | | | | | Rpp21, Stmn2, Thra, Tspan13, |
| | | | | | | Zbtb20, Zfp36l1 |
| c3 | 6 | down | OCT1 06 | 0.000 | 0.001 | Atp13a3, Cyr61, Eif4e, Foxp1, |
| | | | | | | H3f3b, Hoxb4, Id4, Irx3, Mecom, |
| | | | | | | Nsd1, Palmd, Plscr2, Sat1, Scube3, |
| | | | | | | Sox18, Stmn2, Syne2, Zbtb20 |
| c3 | 6 | down | OCT1 B | 0.003 | 0.019 | Csrnp1, Cyr61, Ebf1, Foxp1, |
| | | | | | | H3f3b, Id3, Jund, Med16, Rhob, |
| | | | | | | Skil, Thra, Tspan13 |
| c3 | 6 | down | OCT1 Q5 01 | 0.011 | 0.043 | Arf6, Csrnp1, Cyr61, Ebf1, Foxp1, |
| | | | | | | H3f3b, Hoxa5, Hoxb4, Jund, |
| | | | | | | Med16, Rhob, Rpp21, Srsf2, Thra, |
| | | | | | | Tspan13 |
| c3 | 6 | down | OCT Q6 | 0.000 | 0.035 | Arf6, Csrnp1, Cyr61, Ebf1, Foxp1, |
| | | | | | | H3f3b, Hoxb4, Jund, Med16, |
| | | | | | | Rhob, Rpp21, Thra, Tspan13 |
| c3 | 6 | down | OLF1 01 | 0.000 | 0.002 | Car7, Cdk9, Cmtm8, Cyr61, Dlg3, |
| | | | | | | Egr1, Gabarapl2, Gas6, H3f3b, |
| | | | | | | Mecom, Rgs3, Scube3, Sema3g, |
| | | | | | | Sgk1, Srsf2 |
| c3 | 6 | down | OSF2 Q6 | 0.009 | 0.038 | Amd1, Azin1, Fam13c, Fermt2, |
| | | | | | | Hey 1, Hoxa5, Hoxb4, Id2, Pcsk5, |
| | | | | | | Pik3r3, Pou2f1, Rpp21, Sgk1, |
| | | | | | | Srrm1, Tob1, Tulp4, Vps45 |
| c3 | 6 | down | PAX2 02 | 0.000 | 0.013 | Cited2, Fos, Hoxb4, Mgll, Mn1, |
| | | | | | | Nedd9, Nfkbia, Scube3, Slc20a1, |
| | | | | | | Stmn2, Tob1, Tspan13 |
| c3 | 6 | down | PAX3 B | 0.000 | 0.007 | Dynll1, Fbln5, Hoxc6, Junb, Klf2, |
| | | | | | | Nr4a1, Pnrc1, Ppp1r10, Tbx3 |
| c3 | 6 | down | PAX4 04 | 0.002 | 0.031 | Adcyap1, Arid4a, Ebf1, Foxp1, |
| | | | | | | Hlx, Hoxa5, Hoxb4, Klf7, |
| | | | | | | Map3k11, Plpp3, Ppp1r10, Rgs3, |
| | | | | | | Socs1, Zbtb20 |
| c3 | 6 | down | PAX Q6 | 0.000 | 0.007 | Amd1, Atf3, Btg1, Cyr61, Dlg3, |
| | | | | | | Ebf1, Hoxa5, Ier3, Irx3, Jag1, |
| | | | | | | Pcdh17, Rgs3, S1pr2, Skil, |
| | | | | | | Tnfsf10, Tpm3, Upk2 |
| c3 | 6 | down | PITX2 Q2 | 0.005 | 0.037 | App, Cd2ap, Gadd45g, Hoxb4, |
| | | | | | | Ier5, Kitl, Msx1, Ppt2, Rora, Tapbp |
| c3 | 6 | down | POU3F2 01 | 0.012 | 0.031 | Cxcl1, Fgfr4, Nrp1, Nsd1, Tapbp, |
| | | | | | | Tob1 |
| c3 | 6 | down | POU3F2 02 | 0.000 | 0.014 | Ankrd11, Anp32a, Bst2, Cfap20, |
| | | | | | | Cyp4b1, Cyr61, Foxp1, H3f3b, |
| | | | | | | Ing1, Scube3, Smarca2, Srsf7, |
| | | | | | | Tapbp, Ubb |
| c3 | 6 | down | POU6F1 01 | 0.000 | 0.008 | Dusp1, Fgf13, Foxp1, Hoxa5, |
| | | | | | | Hoxb4, Id2, Nsd1, Pcmtd1, Skil, |
| | | | | | | Tob1, Zbtb20 |
| c3 | 6 | down | RAAGNYNNCTTY UNKNOWN | 0.000 | 0.002 | Atf4, Fbln5, Fchsd2, Hoxa5, Id3, |
| | | | (SEQ ID NO: 23) | | | Klf7, Lmo2, Palmd, Pdgfb, |
| | | | | | | Rnf152, Rora, Scube3, Zfp36l1 |
| c3 | 6 | down | RCGCANGCGY NRF1 Q6 (SEQ | 0.000 | 0.041 | Anp32a, Arf6, Arid4a, Brd2, |
| | | | ID NO: 24) | | | Clock, Ddit3, Dnaja1, Dnajb9, |
| | | | | | | Dnm3, Fchsd2, Fos, Fus, Irf2bpl, |
| | | | | | | Jag1, Lrp1, Magix, Map3k11, |
| | | | | | | Med11, Pim3, Pnrc1, Ppp1cb, |
| | | | | | | Ppp1r10, Prdx4, Sap18, Son, |
| | | | | | | Srrm2, Srsf2, Srsf7, Taf7, Tdp2, |
| | | | | | | Tmed5, Topors |
| c3 | 6 | down | RGAANNTTC HSF1 01 | 0.003 | 0.032 | Dnaja1, Dnajb1, Dusp1, Fbln5, |
| | | | | | | Gpbp1, Hes1, Hspa1a, Hspa1b, |
| | | | | | | Hspa8, Hspb1, Id4, Ier5l, Irf1, |
| | | | | | | Irf2bpl, Jun, Lrp1, Mat2a, Mbnl2, |
| | | | | | | Mgll, Pnrc1, Rsrc2, Scube3, |
| | | | | | | Tspan13, Zbtb20 |
| c3 | 6 | down | RP58 01 | 0.000 | 0.007 | Amd1, Anp32a, Atf3, Dlg3, |
| | | | | | | Foxp1, Klf13, Mbnl2, Nr3c1, |
| | | | | | | Nuak1, Pdgfb, Plpp3, Rarg, S1pr2, |
| | | | | | | Tgfbr2 |
| c3 | 6 | down | RSRFC4 01 | 0.002 | 0.019 | Atf3, Car7, Col8a1, Filip1, Foxp1, |
| | | | | | | Hoxb4, Mbnl2, Nr4a1, Pik3r3, |
| | | | | | | Rgs3, Smarca2, Tob2, Tspan13 |
| c3 | 6 | down | RTAAACA FREAC2 01 | 0.000 | 0.003 | Anp32a, Arid4a, Cd2ap, Cdkn1a, |
| | | | | | | Cdkn1c, Cited2, Clstn1, Cyr61, |
| | | | | | | Ddx5, Dusp1, Edn1, Fam13c, Fos, |
| | | | | | | Gabarapl2, H3f3b, Hoxa5, Hoxb4, |
| | | | | | | Id2, Ing1, Junb, Lifr, Lrp1, Magix, |
| | | | | | | Mapk3, Mbnl2, Mcc, Mecom, |
| | | | | | | Mgll, Mn1, Nebl, Nr3c1, Nr4a1, |
| | | | | | | Pcsk5, Pdgfb, Plpp3, Pls3, Pnrc1, |
| | | | | | | Polr2l, Ppp1cb, Prex2, Rarg, |
| | | | | | | Scube3, Smarca2, Socs1, Tcf15, |
| | | | | | | Thra, Tob1, Zfp36l2 |
| c3 | 6 | down | RYTAAWNNNTGAY | 0.000 | 0.009 | Egr1, H3f3b, Mat2a, Srd5a2, |
| | | | UNKNOWN (SEQ ID NO: 25) | | | Stmn2 |
| c3 | 6 | down | SP1 Q2 01 | 0.007 | 0.035 | Ace, Anp32a, Chka, Clstn1, Klf13, |
| | | | | | | Klf7, Map3k11, Mecom, Mn1, |
| | | | | | | Nfe2l1, Tbx3, Thra, Zfp36, Zfp687 |
| c3 | 6 | down | SP1 Q6 | 0.000 | 0.019 | Ace, Anp32a, Chka, Clstn1, |
| | | | | | | Dynll1, Irx3, Klf11, Klf13, Klf2, |
| | | | | | | Map3k11, Taf11, Thra, Timp3 |
| c3 | 6 | down | SP1 Q6 01 | 0.002 | 0.009 | Ace, Anp32a, Ccdc85b, Chka, |
| | | | | | | Eps8l2, Fdx1, Fosb, Gabarapl2, |
| | | | | | | Irx3, Klf11, Klf2, Lima1, |
| | | | | | | Map3k11, Slc30a3, Srsf2 |
| c3 | 6 | down | SRF C | 0.005 | 0.019 | Col8a1, Cyr61, Efhd1, Egr1, Fos, |
| | | | | | | Fosb, Foxp1, Gadd45g, Hoxa5, |
| | | | | | | Hoxb4, Ier2, Junb, Klf6, Nr4a1, |
| | | | | | | Rgs3, Tpm3 |
| c3 | 6 | down | SRF Q4 | 0.000 | 0.001 | Atrx, Cited2, Cx3cl1, Edn1, Efhd1, |
| | | | | | | Egr1, Fos, Fosb, Foxp1, Gadd45g, |
| | | | | | | Hoxa5, Hoxb4, Ier2, Junb, Klf6, |
| | | | | | | Mcl1, Msx1, Nr4a1, Nuak1, Plpp3, |
| | | | | | | Scube3, Sgk1, Srd5a2, Tpm3 |
| c3 | 6 | down | SRF Q5 01 | 0.000 | 0.003 | Arf6, Atf3, Atf4, Cited2, Efhd1, |
| | | | | | | Egr1, Fos, Fosb, Foxp1, Gadd45g, |
| | | | | | | Gpbp1, Hoxb4, Ier2, Junb, Klf6, |
| | | | | | | Plpp3, Sgk1, Srd5a2, Timp3, Tpm3 |
| c3 | 6 | down | SRF Q6 | 0.000 | 0.008 | Atf4, Cited2, Cx3cl1, Efhd1, Egr1, |
| | | | | | | Fos, Fosb, Foxp1, Gadd45g, |
| | | | | | | Hoxa5, Hoxb4, Junb, Klf6, Msx1, |
| | | | | | | Nr4a1, Plpp3, Srd5a2, Tpm3, Zeb1 |
| c3 | 6 | down | STAT3 02 | 0.004 | 0.018 | Azin1, Bcam, Ddit3, Egr1, Fosb, |
| | | | | | | Hoxb4, Irf1, Nr4a1, Rgs3, Scube3, |
| | | | | | | Socs1, Upk2 |
| c3 | 6 | down | STAT5A 04 | 0.004 | 0.032 | Aif1l, Cited2, Gadd45g, Gpbp1, |
| | | | | | | Hoxc6, Irx3, Mecom, Nr4a1, Pls3, |
| | | | | | | Ppp1r10, Runx2, Slc50a1 |
| c3 | 6 | down | STTTCRNTTT IRF Q6 (SEQ ID | 0.013 | 0.035 | Arhgap5, Arhgef6, B2m, Bst2, |
| | | | NO: 26) | | | Cbx4, Cited2, Isg15, Lgals3bp, |
| | | | | | | Map3k11, Msx1, Pik3r3, Ppp1r10, |
| | | | | | | Psmb10, Psmb8, Psmb9, Slc12a7, |
| | | | | | | Syne2, Tapbp, Tcf15, Xaf1 |
| c3 | 6 | down | TAAYNRNNTCC UNKNOWN | 0.008 | 0.028 | Arhgap5, Atf3, Azin1, Dis3l, Dll4, |
| | | | (SEQ ID NO: 27) | | | Dolk, Fam181b, Id3, Mgll, Npnt |
| c3 | 6 | down | TACAATC MIR508 | 0.020 | 0.044 | Dnajb1, Fchsd2, Fgf7, Klf4 |
| c3 | 6 | down | TAL1BETAITF2 01 | 0.002 | 0.025 | Cd81, Ddit3, Dll4, Fgf13, Fosb, |
| | | | | | | Foxp1, Gabarapl2, Lima1, Mbnl2, |
| | | | | | | Nsd1, Pdgfb, Pdia6, Plcb1, Rgs3, |
| | | | | | | Zbtb20, Zfp36l1 |
| c3 | 6 | down | TATA C | 0.000 | 0.031 | Atf3, Azin1, Ccdc85b, Col8a1, |
| | | | | | | Cyr61, Ddx5, Epas1, Foxp1, |
| | | | | | | Gadd45g, Grrp1, H3f3b, Hoxb4, |
| | | | | | | Jund, Kitl, Lmo2, Mecom, |
| | | | | | | Ppp1r10, Prdm16, Slc50a1, Tbx3, |
| | | | | | | Tgfb2, Tob1 |
| c3 | 6 | down | TATTATA MIR374 | 0.005 | 0.047 | Amd1, Arhgap5, Arl15, Arl2bp, |
| | | | | | | Cbx4, Ccnl1, Fosb, Foxp1, Hes1, |
| | | | | | | Map4k3, Mbnl2, Msx1, Nfkbiz, |
| | | | | | | Nr3c1, Nuak1, Pnrc1, Runx2, |
| | | | | | | Srsf7, Stmn2 |
| c3 | 6 | down | TAXCREB 01 | 0.000 | 0.008 | Braf, Cd2ap, Dusp1, Fos, Fosb, |
| | | | | | | Irf2bpl, Jund, Klf13, Mbnl2, Pnrc1 |
| c3 | 6 | down | TEF Q6 | 0.003 | 0.016 | Amd1, Ccdc85b, Foxp1, Hey1, |
| | | | | | | Hoxc6, Nrp1, Ppp1r10, Rgs3, |
| | | | | | | Slc20a1, Thap3, Tob1 |
| c3 | 6 | down | TGAYRTCA ATF3 Q6 | 0.000 | 0.043 | Cd2ap, Dusp1, Fos, Gpbp1, Id1, |
| | | | | | | Irf2bpl, Itm2b, Jun, Jund, Mbnl2, |
| | | | | | | Nebl, Pnrc1, Ppp1r15a, Pth, |
| | | | | | | Sipa1l2, Tpm3, Zfp36l1 |
| c3 | 6 | down | TGTTTGY HNF3 Q6 | 0.000 | 0.006 | Btg1, Cited2, Cyr61, Dusp1, |
| | | | | | | Eps8l2, Fam13c, Fchsd2, Fosb, |
| | | | | | | Foxp1, Gadd45b, Id2, Itm2b, Jag1, |
| | | | | | | Junb, Klf6, Klf7, Mapk3, Mast4, |
| | | | | | | Mgll, Mgst1, Mmrn2, Npnt, Nsd1, |
| | | | | | | Palmd, Pdcd4, Pdlim2, Pnrc1, |
| | | | | | | Ptprr, Rgs3, Sgk1, Sipa1l2, |
| | | | | | | Smarca2, Sncg, Sox17, Syne2, |
| | | | | | | Tapbp, Thra, Tob1 |
| c3 | 6 | down | TTCNRGNNNNTTC HSF Q6 | 0.002 | 0.010 | Ahsa1, Dnaja1, Edn1, Fosb, |
| | | | (SEQ ID NO: 8) | | | Foxp1, Hspa1a, Hspa1b, Hspa8, |
| | | | | | | Mat2a, Mecom, Pradc1, Rora, Ubb |
| c3 | 6 | down | TTGCCAA MIR182 | 0.002 | 0.035 | Cd2ap, Cited2, Clock, Cnot6, |
| | | | | | | Efnb2, Epas1, Etl4, Fnbp1l, |
| | | | | | | Irf2bpl, Klf13, Klf7, Mbnl2, |
| | | | | | | Mfap3, Nuak1, Pcdh17, Pcmtd1, |
| | | | | | | Rarg, Rdx, Tob1, Zfp36, Zfp36l1, |
| | | | | | | Znrf1 |
| c3 | 6 | down | TTGGAGA MIR5155P MIR519E | 0.006 | 0.032 | Btg2, Cyr61, Id3, Pou2f1, Pum1, |
| | | | | | | Sertad1, Tgfbr2, Zfp36l1 |
| c3 | 6 | down | TTTGCAC MIR19A MIR19B | 0.000 | 0.042 | Arhgap5, Atrx, Btg1, Cbx1, |
| | | | | | | Ccdc88a, Ccnl1, Cdk19, Clock, |
| | | | | | | Dnajb1, Efnb2, Etl4, Fam46b, |
| | | | | | | Foxp1, Id4, Klf13, Mbnl2, Pik3r3, |
| | | | | | | Plcb1, Pnrc1, Rfx1, Rhob, Sgk1, |
| | | | | | | Smarca2, Socs1, Socs3, Syt11, |
| | | | | | | Tgfbr2 |
| c3 | 6 | down | TTTGTAG MIR520D | 0.002 | 0.019 | Atf4, Cbx4, Cited2, Fnbp1l, |
| | | | | | | Gpatch8, H3f3b, Hes1, Ier5, Klf11, |
| | | | | | | Lysmd2, Mbnl2, Mllt10, Nebl, |
| | | | | | | Nedd9, Pcmtd1, Pdcd4, Ppp1cb, |
| | | | | | | Pum1, Rdx, Rfx1, Son, Tpst1 |
| c3 | 6 | down | TTTNNANAGCYR UNKNOWN | 0.002 | 0.028 | Car7, Cnn1, Foxp1, Hoxb4, Id4, |
| | | | (SEQ ID NO: 28) | | | Lima1, Nrp1 |
| c3 | 6 | down | USF C | 0.015 | 0.048 | Ak3, Arf6, Dnajb9, Dolk, Efnb1, |
| | | | | | | Fchsd2, Ier5l, Mat2a, Polr2l, |
| | | | | | | Slc20a1, Suclg2 |
| c3 | 6 | down | VDR Q3 | 0.014 | 0.042 | Bcam, Ebf1, Fos, Jund, Lrp1, |
| | | | | | | Mecom, Pradc1, Rarg, Slc26a10, |
| | | | | | | Tcf15, Thra, Timp3 |
| c3 | 6 | down | WCTCNATGGY UNKNOWN | 0.000 | 0.018 | Id1, Ier2, Jun, Pradc1, Secisbp2l, |
| | | | (SEQ ID NO: 29) | | | Skil |
| c3 | 6 | down | WGGAATGY TEF1 Q6 | 0.003 | 0.038 | Atf4, Bcam, Cyr61, Ddit3, Dlg3, |
| | | | | | | Edn1, Id2, Ier5, Jund, Kcne2, Lifr, |
| | | | | | | Mbnl2, Mgll, Nr4a1, Nsd1, Pls3, |
| | | | | | | Plscr2, Rarg, Rgs3, Smarca2 |
| c3 | 6 | down | WHN B | 0.000 | 0.001 | Amd1, Azin1, Ddx5, Dll4, Dolk, |
| | | | | | | Ebf1, Efnb1, H3f3b, Id2, Jag1, Jun, |
| | | | | | | Junb, Klf2, Slc20a1, Sncb, Ssb, |
| | | | | | | Tbx3, Thra, Tob1, Tob2 |
| c3 | 6 | down | YATGNWAAT OCT C | 0.005 | 0.044 | Bst2, Cyr61, Dll4, Foxp1, H3f3b, |
| | | | | | | Hoxa5, Hoxb4, Id2, Ing1, Jund, |
| | | | | | | Mbnl2, Mcc, Med16, Rhob, Thra, |
| | | | | | | Tspan13 |
| c3 | 6 | down | YATTNATC UNKNOWN | 0.000 | 0.014 | App, Csrnp1, Ctbp2, Fam71a, |
| | | | | | | H3f3b, Hoxb4, Id2, Irx3, Mbnl2, |
| | | | | | | Mn1, Nsd1, Palmd, Pcsk5, |
| | | | | | | Ppp1r10, Sat1, Scube3, Smarca2, |
| | | | | | | Stmn2 |
| c3 | 6 | down | YGCANTGCR UNKNOWN | 0.016 | 0.035 | App, Arhgap5, Clstn1, Fgf13, |
| | | | | | | Mat2a, Slc9a3r2, Topors |
| c3 | 6 | down | YTATTTTNR MEF2 02 | 0.000 | 0.011 | Arl6ip5, Car7, Cdkn1a, Col8a1, |
| | | | | | | Cxcl1, Dll4, Ebf1, Edn1, Fos, |
| | | | | | | Fosb, Foxp1, Gadd45b, Grrp1, |
| | | | | | | Hes1, Hlx, Hoxa5, Id2, Ier5l, Jun, |
| | | | | | | Junb, Lifr, Mbnl2, Mgll, Npnt, |
| | | | | | | Nr4a1, Pcmtd1, Pdcd4, Ppp1r10, |
| | | | | | | Ppp1r16b, Rpp21, Scube3, |
| | | | | | | Smarca2, Tnfsf10, Usp25, Vps45 |
| c3 | 6 | down | YWATTWNNRGCT UNKNOWN | 0.008 | 0.025 | Atf3, Hey1, Nfkbia, Nr4a1, Pik3r3, |
| | | | (SEQ ID NO: 30) | | | Sptssa |
| c3 | 6 | down | YYCATTCAWW UNKNOWN | 0.000 | 0.000 | Atf3, Azin1, Ckb, Cyr61, Ddx5, |
| | | | (SEQ ID NO: 31) | | | Eif4e, Filip1, Fosb, Id1, Mcc, |
| | | | | | | Plpp3, Rgs3, Tcf15, Tob1, Tpm3, |
| | | | | | | Tspan13 |
| c3 | 6 | down | ZF5 B | 0.003 | 0.020 | Car7, Cdk9, Clock, Ddit3, Dll4, |
| | | | | | | Fgf13, Junb, Magix, Mat2a, Pdgfb, |
| | | | | | | Plcb1, Slc26a10, Thra, Tra2b |
| c3 | 6 | down | ZIC2 01 | 0.010 | 0.035 | Adamts1, Ckb, Dll4, Dynll1, |
| | | | | | | Foxp1, Hoxb4, Irf9, Lrp1, Pdlim2, |
| | | | | | | Ppp1r16b, Rarg, Thap3, Timp3, |
| | | | | | | Tob2, Zfp36l1 |
| c4 | 0 | down | GNF2 CD97 | 0.004 | 0.020 | Id2, Iqgap1, Jak1, Ptger2 |
| c4 | 0 | down | MODULE 106 | 0.004 | 0.008 | Cyp2c38, Cyp4b1 |
| c4 | 0 | down | MODULE 123 | 0.000 | 0.001 | Aplnr, Cited2, Egr1, Ets2, Fos, |
| | | | | | | Gpr182, Id4, Jun, Junb, Jund, Klf6, |
| | | | | | | Nr3c1, Pparg, Ptger2 |
| c4 | 0 | down | MODULE 173 | 0.000 | 0.004 | Dusp6, Gadd45g, Rgs3, Rgs4 |
| c4 | 0 | down | MODULE 197 | 0.000 | 0.002 | Atf4, Cited2, Egr1, Ets2, Fos, |
| | | | | | | Hif1a, Id4, Jun, Junb, Smarca2 |
| c4 | 0 | down | MODULE 407 | 0.000 | 0.005 | Dusp6, Gadd45g, Rgs3, Rgs4 |
| c4 | 0 | down | MODULE 465 | 0.000 | 0.014 | Dusp6, Rgs4 |
| c4 | 0 | down | MODULE 537 | 0.000 | 0.015 | Birc3, Ier3, Nrg2, Son |
| c4 | 0 | up | MODULE 153 | 0.000 | 0.017 | Emp1, Fabp5, Plod1 |
| c4 | 0 | up | MODULE 155 | 0.001 | 0.031 | Fkbp1a, Ppic |
| c4 | 0 | up | MODULE 159 | 0.000 | 0.014 | Eef1a1, Eef2, Eif4b, Eif4h, Gnai2, |
| | | | | | | Rab11a, Rab5c |
| c4 | 0 | up | MODULE 245 | 0.004 | 0.040 | Fkbp1a, Ppic |
| c4 | 0 | up | MODULE 306 | 0.000 | 0.011 | Ldha, Pgam1, Pgk1, Sdha, Tpi1 |
| c4 | 0 | up | MODULE 470 | 0.000 | 0.035 | Igfbp5 |
| c4 | 0 | up | MODULE 86 | 0.001 | 0.038 | Kras, Rab11a, Rab5c, Rras |
| c4 | 0 | up | MODULE 87 | 0.000 | 0.007 | Eef1a1, Eef2, Rab11a, Rab5c |
| c4 | 0 | up | MORF SART1 | 0.000 | 0.035 | Clic1, Eef2, Gdi2 |
| c4 | 15 | up | MODULE 191 | 0.004 | 0.044 | Fth1, Mt2 |
| c4 | 16 | up | MORF WNT1 | 0.001 | 0.033 | 1700025G04Rik, Art3, Cul3, |
| | | | | | | Epha7, Foxn2, Hoxd9, Kif1a, |
| | | | | | | Map3k14, Odf2, Phkg2, Syt11 |
| c4 | 1 | down | MODULE 355 | 0.000 | 0.008 | Dnajb1, Hspa5, Hspa8, Tbca |
| c4 | 1 | down | MODULE 433 | 0.000 | 0.000 | Bmp4, Csf1, Cxcl1, Cxcl12, Ereg, |
| | | | | | | Fgf7, Il15, Mdk, Ogn, Vegfc |
| c4 | 1 | up | MODULE 190 | 0.000 | 0.006 | Igfbp3, Igfbp5, Ltbp1, Wisp2 |
| c4 | 1 | up | MODULE 210 | 0.000 | 0.008 | Aebp1, Mmp11, Timp1 |
| c4 | 1 | up | MODULE 562 | 0.002 | 0.007 | Mmp11, Timp1, Timp2 |
| c4 | 2 | down | GCM BMPR2 | 0.005 | 0.026 | Asap2, Calm1, Map4k4, Nfia, |
| | | | | | | Rtn4, Vps26b, Wsb2 |
| c4 | 2 | down | GCM CALM1 | 0.001 | 0.040 | Asap2, Calm1, Clasp2, Map4k4, |
| | | | | | | Naa30, Rtn4, Vps26b, Wsb2, |
| | | | | | | Ywhag |
| c4 | 2 | down | GCM MAP4K4 | 0.000 | 0.008 | 4932438A13Rik, Calm1, Casc4, |
| | | | | | | Ccdc88a, Clasp2, Il17d, Lrrc8a, |
| | | | | | | Map4k4, Marf1, Naa30, Pitpnc1, |
| | | | | | | Rtn4, Ubl3, Wsb1, Wsb2, Ywhag |
| c4 | 2 | down | GCM NCAM1 | 0.000 | 0.008 | Asap2, Calm1, Clasp2, Kat6b, |
| | | | | | | Naa30, Scarb2, Smad7, Ube2n, |
| | | | | | | Vps26b, Wsb2, Zfand5 |
| c4 | 2 | down | MODULE 138 | 0.000 | 0.026 | Erf, Fxyd3, Fzd2, S100a1, Tnni2, |
| | | | | | | Tob1, Zfr |
| c4 | 2 | down | MODULE 174 | 0.000 | 0.003 | Fxyd3, Fzd2, Ier3, Kdm5c, Ninj1, |
| | | | | | | Piezo1, Plod1, Ppib, S100a1, |
| | | | | | | Tnni2, Tob1 |
| c4 | 2 | down | MODULE 241 | 0.000 | 0.043 | Fzd2, Ier3, Kdm5c, Ninj1, Piezo1, |
| | | | | | | Plod1, Ppib, S100a1 |
| c4 | 2 | down | MODULE 257 | 0.001 | 0.038 | Akt1, Dnajc3, Eps8l2, Erf, |
| | | | | | | Fam168a, Gps1, Hmg20b, Inppl1, |
| | | | | | | Kmt2d, Lrrc8a, Myh7b, Ppp1r15a, |
| | | | | | | Sh3glb2, Ski, Slc20a1, Taf6l, |
| | | | | | | Ubtd1 |
| c4 | 2 | down | MODULE 291 | 0.000 | 0.045 | Fzd2, Ier3, Kdm5c, Ninj1, Piezo1, |
| | | | | | | Plod1, Ppib, S100a1 |
| c4 | 2 | down | MODULE 321 | 0.002 | 0.009 | 1110008P14Rik, Actn4, Asap2, |
| | | | | | | Btg3, Ckap4, Ext1, Lrrc8a, Nrp2, |
| | | | | | | Phlda1, Plod1, Prnp, S100a10, |
| | | | | | | Spats2l, Tgfbr2, Twsg1 |
| c4 | 2 | down | MORF AP2M1 | 0.000 | 0.009 | 1110008P14Rik, Actn4, Calm1, |
| | | | | | | Calm2, Clic1, Cox6a1, Eif4g2, |
| | | | | | | Huwe1, Jtb, Ndufa1, Pgam1, |
| | | | | | | Piezo1, Polr2l, Rab1a, Sap18, |
| | | | | | | Slc4a2, Srp9, Tmbim6, Tmed2, |
| | | | | | | Uqcrb, Ywhaq |
| c4 | 2 | down | MORF CDC10 | 0.001 | 0.025 | Arl2bp, Calm2, Hnrnph3, Ppp2ca, |
| | | | | | | Ptp4a2, Sec63, Sept7, Tacc1, |
| | | | | | | Ywhaq |
| c4 | 2 | down | MORF DDB1 | 0.000 | 0.018 | Abr, Actn4, Calm2, Drap1, |
| | | | | | | Huwe1, Mtx1, Pcgf2, Pde6d, |
| | | | | | | Rab1a, Rabac1, Scarb1, Sec62, |
| | | | | | | Sept7, Slc25a11, Slc4a2, Snapc5, |
| | | | | | | Tmed1, Vgll4, Ywhaq |
| c4 | 2 | down | MORF PRKAG1 | 0.000 | 0.000 | Abr, Calm2, Drap1, Huwe1, |
| | | | | | | Kmt2d, Mtx1, P4ha1, Rab1a, |
| | | | | | | Rabac1, Rtn4, Scarb1, Sec62, |
| | | | | | | Sec63, Sept7, Slc25a11, Slc4a2, |
| | | | | | | Tmed1, Vgll4, Yif1a, Ywhaq |
| c4 | 2 | down | MORF PTPN11 | 0.002 | 0.040 | Akr7a5, Atp5j, Baz1b, Cbfb, Cycs, |
| | | | | | | Ifrd1, Ndufc1, Ndufs6, Prrc2c, |
| | | | | | | Ptges3, Snrpd3, Ube2n |
| c4 | 2 | down | MORF SP3 | 0.000 | 0.008 | BC005537, Bzw1, Calm2, Phf3, |
| | | | | | | Rtn4, Srp9, Tmed2, Ubxn4, Vgll4, |
| | | | | | | Ywhaq |
| c4 | 3 | down | GNF2 MSN | 0.002 | 0.039 | Clic1, Emp3, Iqgap1 |
| c4 | 3 | down | MODULE 155 | 0.000 | 0.015 | Dnaja1, Hspa8, Kdelr1, Kdelr2, |
| | | | | | | Pdia6, Ssr1, Ssr2 |
| c4 | 3 | down | MODULE 245 | 0.000 | 0.019 | Dnaja1, Hspa8, Kdelr1, Kdelr2, |
| | | | | | | Pdia6, Ssr1, Ssr2 |
| c4 | 3 | down | MODULE 299 | 0.000 | 0.043 | Atox1, Bag1, Cryab, Dnaja1, |
| | | | | | | Hsp90b1, Hspa5, Hspa8, Pdia6, |
| | | | | | | Serpinh1 |
| c4 | 3 | down | MODULE 355 | 0.000 | 0.011 | Bag1, Cryab, Dnaja1, Hsp90b1, |
| | | | | | | Hspa1l, Hspa5, Hspa8 |
| c4 | 3 | up | GNF2 CD1D | 0.002 | 0.045 | Cd14, Cd302, Mafb, Psap, |
| | | | | | | Tnfrsf1b |
| c4 | 3 | up | GNF2 HLA-C | 0.000 | 0.040 | B2m, H2-M3, Tsc22d3 |
| c4 | 3 | up | MODULE 210 | 0.000 | 0.031 | Aebp1, Mmp14, Mmp2 |
| c4 | 3 | up | MODULE 361 | 0.000 | 0.045 | Bcl2, Btg2, Cd200, Ctsf, Dbp, |
| | | | | | | Prg4, Txnip, Zfp36l2 |
| c4 | 5 | down | MORF AP2M1 | 0.000 | 0.000 | Atox1, Calm1, Calm2, Calr, Clic1, |
| | | | | | | Hspa8, Ldha, Ndufa1, Snx3, |
| | | | | | | Ywhaq |
| c4 | 5 | down | MORF CSNK2B | 0.003 | 0.038 | Atp6ap1, Bzw1, Chd4, Cox6c, |
| | | | | | | Cycs, Dynll1, Ifrd1, Ndufs5, Snx3, |
| | | | | | | Ywhaq |
| c4 | 5 | down | MORF EIF3S2 | 0.000 | 0.049 | Alg3, Atf4, Cdv3, Cycs, Hsp90b1, |
| | | | | | | Ifrd1, Ldha, Ndufs5, Pdia6, Ywhaq |
| c4 | 5 | down | MORF GNB1 | 0.000 | 0.036 | Arpc3, Atf4, Atp6ap1, Calm2, |
| | | | | | | Chd4, Dynll1, Ifrd1, Ndufs5, |
| | | | | | | Pdia6, Snx3, Ywhaq |
| c4 | 5 | down | MORF RAD23A | 0.000 | 0.046 | Atp6ap1, Bzw1, Chd4, Cycs, |
| | | | | | | Dynll1, Ifrd1, Ndufs5, Pdia6, |
| | | | | | | Snx3, Ywhaq |
| c4 | 5 | down | MORF UBE2I | 0.005 | 0.042 | Aatf, Arpc3, Atf4, Calm2, Cdv3, |
| | | | | | | Chd4, Clic1, Ldha, Oaz1, Rhoa, |
| | | | | | | Ywhaq |
| c4 | 5 | up | GNF2 CD14 | 0.004 | 0.030 | Cd302, Fgl2, Psap |
| c4 | 5 | up | GNF2 HLA-C | 0.000 | 0.022 | B2m, Tsc22d3 |
| c4 | 5 | up | GNF2 IGFBP1 | 0.002 | 0.034 | Gm4450, Mmp11, Svep1 |
| c4 | 5 | up | GNF2 KISS1 | 0.002 | 0.023 | Cdkn1c, Egfr, Gm4450, Mmp11, |
| | | | | | | Svep1 |
| c4 | 5 | up | GNF2 TIMP2 | 0.000 | 0.001 | Adm, Cdkn1c, Egfr, Gm4450, |
| | | | | | | Mmp11, Svep1 |
| c4 | 5 | up | MODULE 200 | 0.002 | 0.036 | Cyp1b1, Fgfr1, Pdgfrb |
| c4 | 5 | up | MODULE 210 | 0.000 | 0.006 | Aebp1, Mmp11, Mmp14, Mmp2, |
| | | | | | | Timp1 |
| c4 | 5 | up | MODULE 562 | 0.001 | 0.036 | Mmp11, Mmp14, Mmp2, Timp1 |
| c4 | 6 | down | GNF2 DDX5 | 0.002 | 0.038 | Ccnl1, Ddx5, H3f3b, Rsrc2, |
| | | | | | | Scaf11, Ubxn4 |
| c4 | 6 | down | MODULE 121 | 0.000 | 0.003 | Bst2, Btg1, Cd81, Crip1, Cxcl1, |
| | | | | | | Cyr61, Edn1, Gas6, Klf6, Lrp1, |
| | | | | | | Nrp1, Osmr, Pdgfb, Vegfc, |
| | | | | | | Zfp36l2 |
| c4 | 6 | down | MODULE 123 | 0.000 | 0.004 | Atf3, Cbx4, Cdkn1a, Cited2, |
| | | | | | | Cxcl1, Egr1, Fos, Id4, Irf1, Irf2, |
| | | | | | | Jun, Junb, Jund, Klf11, Klf6, Klf7, |
| | | | | | | Msx1, Nr3c1, Pou2f1, Taf7 |
| c4 | 6 | down | MODULE 169 | 0.000 | 0.003 | Crip2, Fos, Id2, Ier3, Isg15, Lmo2, |
| | | | | | | Psmb8, Rgs16, Son |
| c4 | 6 | down | MODULE 197 | 0.000 | 0.000 | Atf3, Atf4, Cbx4, Cdkn1c, Cited2, |
| | | | | | | Cxcl1, Egr1, Fos, Hoxc6, Id4, Jun, |
| | | | | | | Junb, Klf11, Smarca2, Taf7 |
| c4 | 6 | down | MODULE 198 | 0.000 | 0.004 | Anp32a, Atf4, Brd2, Cbx1, Cbx4, |
| | | | | | | Cdkn1a, Ddx5, Fos, Fus, Jun, Junb, |
| | | | | | | Lmo2, Msx1, Ncbp2, Srsf2, Srsf7, |
| | | | | | | Ssb, Taf7, Tra2b, Zfp36l1 |
| c4 | 6 | down | MODULE 213 | 0.000 | 0.004 | Abca3, Ephx1, Id2, Id4, Junb, |
| | | | | | | Tob1 |
| c4 | 6 | down | MODULE 220 | 0.000 | 0.020 | Bst2, Cxcl1, Efnb1, Efnb2, Emp2, |
| | | | | | | Epas1, Epha4, Hoxa5, Id1, Id3, |
| | | | | | | Jag1, Lmo2, Msx1, Nrp1, Ptprr, |
| | | | | | | Rarg, S100a1, Stmn2, Tnfsf12, |
| | | | | | | Utrn |
| c4 | 6 | down | MODULE 223 | 0.000 | 0.001 | B2m, Bst2, Btg1, Cd81, Crip1, |
| | | | | | | Cxcl1, Cyr61, Edn1, Emp2, Gas6, |
| | | | | | | Hfe, Klf6, Lrp1, Nrp1, Osmr, |
| | | | | | | Pdgfb, Zfp36l2 |
| c4 | 6 | down | MODULE 252 | 0.000 | 0.001 | Cbx1, Cbx4, Cdkn1a, Ddx5, Fos, |
| | | | | | | Jun, Junb, Lmo2, Msx1, Ncbp2, |
| | | | | | | Srsf2, Srsf7, Ssb, Taf7, Tra2b, |
| | | | | | | Zfp36l1 |
| c4 | 6 | down | MODULE 46 | 0.000 | 0.002 | B2m, Bst2, Btg1, Cd81, Crip1, |
| | | | | | | Cx3cl1, Cxcl1, Cyr61, Edn1, |
| | | | | | | Emp2, Gas6, Hfe, Ifitm3, Irf1, Irf7, |
| | | | | | | Isg15, Klf6, Lgals3bp, Nfkbia, |
| | | | | | | Nrp1, Osmr, Pdgfb, Psmb9, S1pr2, |
| | | | | | | Tnfsf10, Tnfsf12, Zfp36l2 |
| c4 | 6 | down | MODULE 75 | 0.000 | 0.000 | B2m, Bst2, Btg1, Btg2, Cd81, |
| | | | | | | Cdkn1a, Crip1, Cx3cl1, Cxcl1, |
| | | | | | | Cyr61, Edn1, Emp2, Ephx1, Gas6, |
| | | | | | | Hfe, Ifitm3, Irf1, Irf7, Isg15, Klf6, |
| | | | | | | Lgals3bp, Lrp1, Nfkbia, Nrp1, |
| | | | | | | Osmr, Pdgfb, Psmb9, Tnfsf10, |
| | | | | | | Tnfsf12, Zfp36l2 |
| c4 | 6 | down | MODULE 76 | 0.000 | 0.025 | Cx3cl1, Cxcl1, Fn1, Fos, Mgll, |
| | | | | | | Tpst1 |
| c4 | 6 | down | MODULE 98 | 0.000 | 0.018 | Anp32a, Atf4, Brd2, Cbx1, Cbx4, |
| | | | | | | Cdkn1a, Cited2, Ddx5, Egr1, Fos, |
| | | | | | | Foxd2, Fus, Jun, Junb, Klf6, Lmo2, |
| | | | | | | Msx1, Son, Srsf2, Srsf7, Ssb, Taf7, |
| | | | | | | Tra2b, Zfp36l1 |
| c4 | 6 | down | MORF DNMT1 | 0.000 | 0.020 | Anp32a, Fus, Pdia6, Ptges3, |
| | | | | | | Srrm1, Tra2b |
| c4 | 6 | down | MORF PRKACA | 0.004 | 0.035 | Clock, Irf2, Jag1, Ltbp4, Msx1, |
| | | | | | | Oplah, Slc22a6, Tnfrsf25 |
| c4 | 6 | up | MODULE 416 | 0.000 | 0.012 | Aqp1 |
| c4 | 7 | up | GNF2 CASP1 | 0.000 | 0.031 | Dusp1, Ifngr1, Junb, Mafb |
| c4 | 7 | up | GNF2 CD14 | 0.002 | 0.011 | Dusp1, Mafb |
| c4 | 7 | up | GNF2 CD1D | 0.000 | 0.012 | Dusp1, Mafb |
| c4 | 7 | up | GNF2 CD33 | 0.000 | 0.024 | Dusp1, Mafb |
| c4 | 7 | up | GNF2 CD48 | 0.003 | 0.034 | Psmb8, Tapbp |
| c4 | 7 | up | GNF2 HLA-C | 0.000 | 0.013 | Tapbp, Tsc22d3 |
| c4 | 7 | up | GNF2 PECAM1 | 0.000 | 0.003 | Dusp1, Ifngr1, Mafb, Zfp36 |
| c4 | 7 | up | MODULE 107 | 0.000 | 0.000 | Ace, Adam9, Aebp1, Htra1, |
| | | | | | | Mmp11, Prss23, Timp1 |
| c4 | 7 | up | MODULE 118 | 0.000 | 0.012 | Adam9, Cdkn1a, Csrp2, Cyp1b1, |
| | | | | | | Ddit4, Emp1, Gas1, Itgb5, Junb, |
| | | | | | | Lox, Loxl2, Olfml2b, Pdgfra, |
| | | | | | | Prss23, Psmb8, Serpine1, Spp1, |
| | | | | | | Tgm2, Timp3 |
| c4 | 7 | up | MODULE 123 | 0.000 | 0.021 | Cdkn1a, Cited2, Cxcl9, Egr2, Jun, |
| | | | | | | Junb, Jund, Klf6, Stat1 |
| c4 | 7 | up | MODULE 199 | 0.001 | 0.034 | Axl, Cdkn1a, Ifngr1, Il13ra1, |
| | | | | | | Pdgfra, Pdgfrb, Pdk4 |
| c4 | 7 | up | MODULE 210 | 0.000 | 0.000 | Ace, Adam9, Aebp1, Mmp11, |
| | | | | | | Timp1 |
| c4 | 7 | up | MODULE 259 | 0.000 | 0.030 | Axl, Cdkn1a, Ifngr1, Il13ra1, |
| | | | | | | Pdgfra, Pdgfrb, Pdk4 |
| c4 | 7 | up | MODULE 474 | 0.002 | 0.038 | Ctgf, Htra1, Qsox1, Socs3, Wisp2 |
| c4 | 7 | up | MODULE 52 | 0.000 | 0.040 | 8430408G22Rik, Acsl3, Adam9, |
| | | | | | | Cap2, Cdkn1a, Cited2, Csrp2, |
| | | | | | | Ddit4, Emp1, Emp2, Fat1, Filip1l, |
| | | | | | | Gas1, Itgb5, Junb, Lox, Loxl2, |
| | | | | | | Pdgfra, Plec, Pon2, Prss23, Qsox1, |
| | | | | | | Sdc1, Serpine1, Spp1, Tgm2, |
| | | | | | | Timp2, Timp3 |
| c4 | 7 | up | MODULE 562 | 0.000 | 0.000 | Adam9, Mmp11, Timp1, Timp2, |
| | | | | | | Timp3 |
| c4 | 7 | up | MODULE 84 | 0.000 | 0.000 | Aebp1, Capg, Cdkn1a, Col6a1, |
| | | | | | | Col6a3, Cxcl16, Cxcl9, Cyp1b1, |
| | | | | | | Ddit4, Enpp1, Fkbp5, Gas1, Gbp2, |
| | | | | | | Ifngr1, Junb, Mafb, Nupr1, |
| | | | | | | Olfml2b, Pdgfra, Plec, Sdc1, |
| | | | | | | Serpine1, Serping1, Spp1, Stat1, |
| | | | | | | Tgm2, Timp2, Tsc22d3, Tspan4, |
| | | | | | | Zbtb20 |
| c4 | 7 | up | MORF TPT1 | 0.001 | 0.042 | Eef2, Jund |
| c4 | 8 | up | GNF2 HPN | 0.000 | 0.046 | Gstm2, Rarres2, Serping1, |
| | | | | | | Tmem176a, Tmem176b |
| c4 | 8 | up | MODULE 24 | 0.000 | 0.043 | Actn1, Apoe, Cited2, Col4a1, |
| | | | | | | Col5a2, Col6a1, Col6a2, Col6a3, |
| | | | | | | Cyr61, Ddit4, Dhrs3, Dok1, Ecm1, |
| | | | | | | Fst, Gadd45g, Gstm2, Hspg2, |
| | | | | | | Igfbp4, Itgb5, Lrp1, Mmp11, Mt1, |
| | | | | | | Qdpr, Rarres2, Rbp1, Sdc1, |
| | | | | | | Serpine1, Spp1, St6galnac4, Tgm2 |
| c4 | 9 | down | MORF AP2M1 | 0.000 | 0.013 | 1110008P14Rik, Atox1, Atp5j2, |
| | | | | | | Bud31, Calm1, Calr, Clic1, Cops5, |
| | | | | | | Ddost, Fkbp1a, Gnb2, Hmgn1, |
| | | | | | | Hsbp1, Hspa8, Ndufa1, Prkar1a, |
| | | | | | | Sod1, Srp14, Stx4a, Tial1, Vamp3, |
| | | | | | | Ywhaz |
| c5 | 0 | down | GO POSITIVE REGULATION OF | 0.000 | 0.027 | Fos, Hif1a, Jun, Pdgfb |
| | | | PRI MIRNA TRANSCRIPTION | | | |
| | | | FROM RNA POLYMERASE II | | | |
| | | | PROMOTER | | | |
| c5 | 0 | down | GO REGULATION OF FAT | 0.000 | 0.017 | Bmp2, Cebpa, Cebpb, Id2, Id4, |
| | | | CELL DIFFERENTIATION | | | Jdp2, Noct, Pparg, Rarres2 |
| c5 | 0 | up | GO CELLULAR RESPONSE TO | 0.000 | 0.024 | App, Aqp1, Igfbp5, Stc1 |
| | | | CAMP | | | |
| c5 | 0 | up | GO FIBRONECTIN BINDING | 0.000 | 0.027 | Igfbp3, Igfbp5, Itgb1 |
| c5 | 0 | up | GO POSITIVE REGULATION OF | 0.000 | 0.029 | Igfbp3, Igfbp5 |
| | | | INSULIN LIKE GROWTH | | | |
| | | | FACTOR RECEPTOR | | | |
| | | | SIGNALING PATHWAY | | | |
| c5 | 11 | up | GO PLASMA LIPOPROTEIN | 0.000 | 0.044 | Cd36, Ehd1 |
| | | | PARTICLE CLEARANCE | | | |
| c5 | 11 | up | GO REGULATION OF | 0.000 | 0.048 | Cd36, Ehd1, Lpl, Pparg |
| | | | CHOLESTEROL STORAGE | | | |
| c5 | 11 | up | GO TRIGLYCERIDE | 0.000 | 0.046 | Fabp4, Lipe, Lpl, Pnpla2 |
| | | | CATABOLIC PROCESS | | | |
| c5 | 12 | down | GO CELL FATE COMMITMENT | 0.000 | 0.022 | Foxc2, Gas1, Hoxc10, Ifrd1, |
| | | | | | | Kdm6b, Klf4, Mef2c, Notch1, |
| | | | | | | Ppdpf, Six1, Tcf7l2, Tgfb2, Trps1 |
| c5 | 12 | up | GO GLIAL CELL PROJECTION | 0.000 | 0.003 | App, Glul, Pink1 |
| c5 | 15 | down | GO CELLULAR RESPONSE TO | 0.000 | 0.010 | Bak1, Crebbp, Eef1d, Egr1, Hyal2, |
| | | | RADIATION | | | Mdm2, Ppp1r15a, Ptprk |
| c5 | 15 | down | GO REGULATION OF GENE | 0.003 | 0.039 | Arid1b, H3f3b, Kat2a, Kmt2d, |
| | | | EXPRESSION EPIGENETIC | | | Rrp8, Slc50a1, Taf1d, Tnrc6c, |
| | | | | | | Ubr2, Xpo5, Zfp36 |
| c5 | 15 | down | GO REGULATION OF MRNA | 0.000 | 0.048 | Btg2, Cdc73, Hspa8, Ncbp2, |
| | | | METABOLIC PROCESS | | | Papola, Srsf10, Srsf7, Syncrip, |
| | | | | | | Tnrc6c, Tra2b, Zfp36 |
| c5 | 15 | up | GO NEGATIVE REGULATION | 0.000 | 0.047 | Atg5, Klf15 |
| | | | OF PROTEIN ACETYLATION | | | |
| c5 | 1 | down | GO ALPHA BETA T CELL | 0.000 | 0.022 | Cebpb, Il15, Irf1, Rora |
| | | | ACTIVATION | | | |
| c5 | 1 | down | GO BONE MINERALIZATION | 0.002 | 0.033 | Fgfr2, Ibsp, Klf10, Rspo2 |
| c5 | 1 | down | GO BRANCH ELONGATION OF | 0.000 | 0.025 | Bmp4, Fgfr2, Spry1, Tnc, Wnt5a |
| | | | AN EPITHELIUM | | | |
| c5 | 1 | down | GO CELLULAR RESPONSE TO | 0.000 | 0.011 | Adipoq, Ccl2, Dpep1, Gas6, Mt2, |
| | | | DRUG | | | Myc, Pde4b, Ppp1r15a |
| c5 | 1 | down | GO CELLULAR RESPONSE TO | 0.000 | 0.041 | Adamts1, Fzd1, Gas6, Il15, Pim1, |
| | | | NUTRIENT | | | Tnc, Usf2 |
| c5 | 1 | down | GO CELLULAR SODIUM ION | 0.004 | 0.044 | Agt, Sgk1 |
| | | | HOMEOSTASIS | | | |
| c5 | 1 | down | GO DENTATE GYRUS | 0.000 | 0.030 | Mdk, Pten, Smo |
| | | | DEVELOPMENT | | | |
| c5 | 1 | down | GO GROWTH FACTOR | 0.000 | 0.023 | Agt, Bmp4, Csf1, Ctgf, Cxcl1, |
| | | | ACTIVITY | | | Cxcl12, Efemp1, Fgf7, Il34, Il7, |
| | | | | | | Kitl, Mdk, Ogn, Vegfc |
| c5 | 1 | down | GO INTERSTITIAL MATRIX | 0.000 | 0.028 | Abi3bp, Smoc2, Tnc, Vit |
| c5 | 1 | down | GO LUNG MORPHOGENESIS | 0.000 | 0.029 | Bmp4, Celsr1, Fgfr2, Id1, Rspo2, |
| | | | | | | Spry1, Tnc |
| c5 | 1 | down | GO MACROMOLECULAR | 0.002 | 0.028 | Agt, Apoe, Lpl |
| | | | COMPLEX REMODELING | | | |
| c5 | 1 | down | GO MESODERMAL CELL | 0.000 | 0.029 | Bmp4, Fgfr2, Kdm6b, Wnt3a |
| | | | DIFFERENTIATION | | | |
| c5 | 1 | down | GO NEGATIVE REGULATION | 0.000 | 0.046 | Adipoq, Cldn18, Derl2, Fzd1, |
| | | | OF CELLULAR PROTEIN | | | Gas6, Lypla1, Nfkbia, Pid1, Snai2, |
| | | | LOCALIZATION | | | Ubac2 |
| c5 | 1 | down | GO NEGATIVE REGULATION | 0.000 | 0.045 | Adipoq, Derl2, Fzd1, Gas6, |
| | | | OF INTRACELLULAR PROTEIN | | | Lypla1, Nfkbia, Snai2, Ubac2 |
| | | | TRANSPORT | | | |
| c5 | 1 | down | GO NEGATIVE REGULATION | 0.000 | 0.029 | Adipoq, Bmp4, Cldn18, Hlx, Irf1, |
| | | | OF LEUKOCYTE | | | Myc, Pik3r1, Runx1, Trib1 |
| | | | DIFFERENTIATION | | | |
| c5 | 1 | down | GO NEGATIVE REGULATION | 0.000 | 0.026 | C5ar2, Ccl2, Cxcl12, Grem1 |
| | | | OF LEUKOCYTE MIGRATION | | | |
| c5 | 1 | down | GO NEGATIVE REGULATION | 0.000 | 0.023 | Bmp4, Cebpb, Erbb2, Grem1, |
| | | | OF LEUKOCYTE | | | Sftpd |
| | | | PROLIFERATION | | | |
| c5 | 1 | down | GO NEGATIVE REGULATION | 0.000 | 0.025 | Adipoq, Apoa1, Gas6 |
| | | | OF TUMOR NECROSIS FACTOR | | | |
| | | | MEDIATED SIGNALING | | | |
| | | | PATHWAY | | | |
| c5 | 1 | down | GO POSITIVE REGULATION OF | 0.000 | 0.050 | Bmp4, Grem1, Wnt3a |
| | | | CARDIAC MUSCLE CELL | | | |
| | | | DIFFERENTIATION | | | |
| c5 | 1 | down | GO POSITIVE REGULATION OF | 0.000 | 0.036 | Bmp4, Cd40, Rgcc, Sfrp4 |
| | | | EPITHELIAL CELL APOPTOTIC | | | |
| | | | PROCESS | | | |
| c5 | 1 | down | GO POSITIVE REGULATION OF | 0.000 | 0.037 | Agt, Apob, Csf1, Lpl |
| | | | MACROPHAGE DERIVED | | | |
| | | | FOAM CELL | | | |
| | | | DIFFERENTIATION | | | |
| c5 | 1 | down | GO POSITIVE REGULATION OF | 0.000 | 0.029 | Csf1, Il34, Trib1 |
| | | | MACROPHAGE | | | |
| | | | DIFFERENTIATION | | | |
| c5 | 1 | down | GO POSITIVE REGULATION OF | 0.000 | 0.024 | Adipoq, Myc, Pdgfa |
| | | | METANEPHROS | | | |
| | | | DEVELOPMENT | | | |
| c5 | 1 | down | GO POSITIVE REGULATION OF | 0.000 | 0.029 | Eda, Grem1 |
| | | | NF KAPPAB IMPORT INTO | | | |
| | | | NUCLEUS | | | |
| c5 | 1 | down | GO POSITIVE REGULATION OF | 0.000 | 0.023 | Agt, Dvl2, Gas6, Grem1 |
| | | | PROTEIN TYROSINE KINASE | | | |
| | | | ACTIVITY | | | |
| c5 | 1 | down | GO POSITIVE REGULATION OF | 0.000 | 0.028 | Eda, Grem1, Pik3r1 |
| | | | TRANSCRIPTION FACTOR | | | |
| | | | IMPORT INTO NUCLEUS | | | |
| c5 | 1 | down | GO PROTEIN TYROSINE | 0.000 | 0.045 | Afap1l2, Gas6, Grem1 |
| | | | KINASE ACTIVATOR | | | |
| | | | ACTIVITY | | | |
| c5 | 1 | down | GO REGULATION OF BLOOD | 0.000 | 0.045 | Adipoq, Agt, Gas6, Gja1, Npr3, |
| | | | PRESSURE | | | P2rx2, Sgk1, Vegfc |
| c5 | 1 | down | GO REGULATION OF | 0.000 | 0.024 | Ccl2, Cxcl12, Icam1 |
| | | | CELLULAR EXTRAVASATION | | | |
| c5 | 1 | down | GO REGULATION OF CELL | 0.004 | 0.040 | Bmp4, Myc, Pdgfa |
| | | | PROLIFERATION INVOLVED | | | |
| | | | IN KIDNEY DEVELOPMENT | | | |
| c5 | 1 | down | GO REGULATION OF | 0.000 | 0.036 | Bmp4, Chadl, Ctgf, Efemp1, Gli2, |
| | | | CHONDROCYTE | | | Grem1, Snai2 |
| | | | DIFFERENTIATION | | | |
| c5 | 1 | down | GO REGULATION OF | 0.000 | 0.029 | Adamts20, Adamts9, Kitl, Zeb2 |
| | | | DEVELOPMENTAL | | | |
| | | | PIGMENTATION | | | |
| c5 | 1 | down | GO REGULATION OF | 0.000 | 0.024 | Adipoq, Bmp4, Pdgfa |
| | | | GLOMERULUS | | | |
| | | | DEVELOPMENT | | | |
| c5 | 1 | down | GO REGULATION OF | 0.000 | 0.029 | Adipoq, Atp11c, Bmp4, Cebpb, |
| | | | HEMOPOIESIS | | | Csf1, Gas6, Hlx, Il15, Il34, Il7, |
| | | | | | | Irf1, Kitl, Myc, Nfkbia, Pik3r1, |
| | | | | | | Runx1, Trib1 |
| c5 | 1 | down | GO REGULATION OF | 0.000 | 0.028 | Adipoq, Atp11c, Bmp4, Cebpb, |
| | | | LEUKOCYTE | | | Cldn18, Csf1, Gas6, Gli2, Hlx, |
| | | | DIFFERENTIATION | | | Il15, Il34, Il7, Irf1, Kitl, Klf10, |
| | | | | | | Myc, Pik3r1, Runx1, Sart1, Trib1 |
| c5 | 1 | down | GO REGULATION OF | 0.000 | 0.043 | Ccl2, Csf1, Cx3cl1 |
| | | | MACROPHAGE CHEMOTAXIS | | | |
| c5 | 1 | down | GO REGULATION OF | 0.000 | 0.001 | Adipoq, Agt, Csf1, Lpl, Nfkbia, |
| | | | MACROPHAGE DERIVED | | | Ppara |
| | | | FOAM CELL | | | |
| | | | DIFFERENTIATION | | | |
| c5 | 1 | down | GO REGULATION OF | 0.000 | 0.002 | Adipoq, Csf1, Il34, Inhba, Trib1 |
| | | | MACROPHAGE | | | |
| | | | DIFFERENTIATION | | | |
| c5 | 1 | down | GO REGULATION OF | 0.000 | 0.027 | Csf1, Hoxa7, Il34, Myc |
| | | | MONOCYTE | | | |
| | | | DIFFERENTIATION | | | |
| c5 | 1 | down | GO REGULATION OF | 0.000 | 0.041 | Adipoq, Cebpb, Cldn18, Csf1, |
| | | | MYELOID CELL | | | Il34, Kitl, Myc, Nfkbia, Pik3r1, |
| | | | DIFFERENTIATION | | | Runx1, Trib1 |
| c5 | 1 | down | GO REGULATION OF | 0.000 | 0.021 | Adipoq, Cebpb, Cldn18, Csf1, |
| | | | MYELOID LEUKOCYTE | | | Il34, Kitl, Myc, Pik3r1, Runx1, |
| | | | DIFFERENTIATION | | | Trib1 |
| c5 | 1 | down | GO REGULATION OF NF | 0.000 | 0.021 | Eda, Grem1, Nfkbia, Nfkbib, |
| | | | KAPPAB IMPORT INTO | | | Ppm1a, Prdx1 |
| | | | NUCLEUS | | | |
| c5 | 1 | down | GO REGULATION OF | 0.000 | 0.030 | Bmp2, Bmp4, Csf1, Rspo2 |
| | | | ODONTOGENESIS | | | |
| c5 | 1 | down | GO REGULATION OF | 0.000 | 0.028 | Bmp2, Bmp4, Csf1, Rspo2 |
| | | | ODONTOGENESIS OF DENTIN | | | |
| | | | CONTAINING TOOTH | | | |
| c5 | 1 | down | GO REGULATION OF PLASMA | 0.000 | 0.028 | Adipoq, Agt, Apoe, Lpl |
| | | | LIPOPROTEIN PARTICLE | | | |
| | | | LEVELS | | | |
| c5 | 1 | down | GO RENAL SYSTEM PROCESS | 0.000 | 0.027 | Cyp4f14, Gas6, Gja1 |
| | | | INVOLVED IN REGULATION | | | |
| | | | OF BLOOD VOLUME | | | |
| c5 | 1 | down | GO RENAL SYSTEM PROCESS | 0.000 | 0.028 | Agt, Cyp4f14, Gas6, Gja1 |
| | | | INVOLVED IN REGULATION | | | |
| | | | OF SYSTEMIC ARTERIAL | | | |
| | | | BLOOD PRESSURE | | | |
| c5 | 1 | down | GO RESPONSE TO ANTIBIOTIC | 0.000 | 0.029 | Ccl2, Hspa5, Hyal1, Id1, Ppp1r15a |
| c5 | 1 | down | GO RESPONSE TO NITRIC | 0.006 | 0.045 | Bmp4, Dpep1, Kcnc2 |
| | | | OXIDE | | | |
| c5 | 1 | down | GO SMOOTH MUSCLE TISSUE | 0.000 | 0.027 | Bmp4, Dlg1, Mylk, Tiparp, Zfand5 |
| | | | DEVELOPMENT | | | |
| c5 | 1 | down | GO SPECIFICATION OF ORGAN | 0.002 | 0.035 | Bmp4, Fgfr2, Gli3 |
| | | | IDENTITY | | | |
| c5 | 1 | down | GO SULFATE TRANSPORT | 0.004 | 0.036 | Slc26a11, Slc26a7 |
| c5 | 1 | up | GO CELLULAR RESPONSE TO | 0.002 | 0.041 | Cdkn1a, Hspa1a |
| | | | HEAT | | | |
| c5 | 1 | up | GO DECIDUALIZATION | 0.000 | 0.004 | Ctsb, Ctsl, Dedd, Ptgis, Vdr |
| c5 | 1 | up | GO EPITHELIAL CELL CELL | 0.000 | 0.044 | Ctnna1, Cyp1b1, Itgb5 |
| | | | ADHESION | | | |
| c5 | 1 | up | GO EXTRACELLULAR MATRIX | 0.000 | 0.040 | Ctsl, Fbn1, Mmp11, Timp1, Timp2 |
| | | | DISASSEMBLY | | | |
| c5 | 1 | up | GO FIBRINOLYSIS | 0.000 | 0.044 | Anxa2, Pros1, Serpine1, Serping1 |
| c5 | 1 | up | GO FIBRONECTIN BINDING | 0.000 | 0.040 | Igfbp3, Igfbp5, Sdc4, Sfrp2, Vegfa |
| c5 | 1 | up | GO INTRAMOLECULAR | 0.000 | 0.042 | Mif, Ptgis, Tpi1 |
| | | | OXIDOREDUCTASE ACTIVITY | | | |
| c5 | 1 | up | GO INTRINSIC APOPTOTIC | 0.000 | 0.042 | Cdkn1a, Ddit4, Nupr1, Sirt1 |
| | | | SIGNALING PATHWAY IN | | | |
| | | | RESPONSE TO DNA DAMAGE | | | |
| | | | BY P53 CLASS MEDIATOR | | | |
| c5 | 1 | up | GO MATERNAL PLACENTA | 0.000 | 0.045 | Ctsb, Ctsl, Dedd, Ptgis, Vdr |
| | | | DEVELOPMENT | | | |
| c5 | 1 | up | GO NADH METABOLIC | 0.002 | 0.039 | Eno1, Pfkl, Pgam1, Tpi1 |
| | | | PROCESS | | | |
| c5 | 1 | up | GO NEGATIVE REGULATION | 0.000 | 0.036 | Anxa2, Kng2, Pros1, Serpine1, |
| | | | OF COAGULATION | | | Serping1 |
| c5 | 1 | up | GO NEGATIVE REGULATION | 0.000 | 0.044 | Cst3, Cygb, Errfi1 |
| | | | OF MULTICELLULAR | | | |
| | | | ORGANISMAL METABOLIC | | | |
| | | | PROCESS | | | |
| c5 | 1 | up | GO POSITIVE REGULATION OF | 0.000 | 0.020 | Fmn1, Itgb1bp1, Rock1, Sdc4, |
| | | | ADHERENS JUNCTION | | | Vegfa, Wnt4 |
| | | | ORGANIZATION | | | |
| c5 | 1 | up | GO POSITIVE REGULATION OF | 0.000 | 0.032 | Mif, Tnfsf11 |
| | | | ORGANIC ACID TRANSPORT | | | |
| c5 | 1 | up | GO PROSTANOID | 0.000 | 0.040 | Mif, Ptgis |
| | | | BIOSYNTHETIC PROCESS | | | |
| c5 | 1 | up | GO PROTEASE BINDING | 0.000 | 0.005 | Anxa2, Cst3, Ecm1, Lrp1, Pink1, |
| | | | | | | Serpine1, Timp1, Timp2 |
| c5 | 1 | up | GO REGULATION OF FATTY | 0.000 | 0.033 | Mif, Tnfsf11 |
| | | | ACID TRANSPORT | | | |
| c5 | 1 | up | GO REGULATION OF | 0.000 | 0.002 | Mif, Tnfsf11 |
| | | | ICOSANOID SECRETION | | | |
| c5 | 1 | up | GO RESPONSE TO INCREASED | 0.000 | 0.019 | Bnip3, Cdkn1a |
| | | | OXYGEN LEVELS | | | |
| c5 | 2 | down | GO KINASE INHIBITOR | 0.000 | 0.001 | Bgn, Cdkn1a, Dnajc3, Hspb1, |
| | | | ACTIVITY | | | Pkig, Socs1, Socs2, Socs3, Socs5, |
| | | | | | | Socs7, Trib1, Wnk1, Ywhag |
| c5 | 2 | down | GO KINASE REGULATOR | 0.000 | 0.000 | Bgn, Calm1, Calm2, Ccnl1, |
| | | | ACTIVITY | | | Cdkn1a, Dnajc3, Hspb1, Klf4, |
| | | | | | | Pkig, Rgcc, Socs1, Socs2, Socs3, |
| | | | | | | Socs5, Socs7, Trib1, Wnk1, |
| | | | | | | Ywhag |
| c5 | 2 | down | GO MIDBRAIN DEVELOPMENT | 0.000 | 0.034 | Atp5j, Calm1, Calm2, Dynll1, |
| | | | | | | Fgfr1, Hes1, Mag, Nr4a2, Rhoa, |
| | | | | | | S100a1, Uqcrq, Ywhaq |
| c5 | 2 | down | GO NEGATIVE REGULATION | 0.000 | 0.001 | Bhlhe40, Ctnnbip1, Foxj1, Id1, |
| | | | OF SEQUENCE SPECIFIC DNA | | | Id2, Klf4, Peli1, Pim1, Prnp, Rlim, |
| | | | BINDING TRANSCRIPTION | | | Rnf2, Sfrp5, Smad7, Trib1, Wwp2 |
| | | | FACTOR ACTIVITY | | | |
| c5 | 2 | down | GO NEGATIVE REGULATION | 0.000 | 0.017 | Bambi, Cd109, Chst11, Htra1, Ski, |
| | | | OF TRANSFORMING GROWTH | | | Skil, Smad7, Snx6, Tgfbr2, Vasn |
| | | | FACTOR BETA RECEPTOR | | | |
| | | | SIGNALING PATHWAY | | | |
| c5 | 3 | down | GO CELLULAR RESPONSE TO | 0.000 | 0.015 | Crip1, Hspa5, Scn3a |
| | | | ANTIBIOTIC | | | |
| c5 | 3 | down | GO ENDOPLASMIC | 0.000 | 0.014 | Dnajb11, Hsp90b1, Hspa5, Pdia6 |
| | | | RETICULUM CHAPERONE | | | |
| | | | COMPLEX | | | |
| c5 | 3 | down | GO ENDOPLASMIC | 0.000 | 0.017 | Arsb, Calr, Col15a1, Col1a1, |
| | | | RETICULUM LUMEN | | | Col1a2, Col3a1, Col5a2, Col6a1, |
| | | | | | | Col6a2, Col6a3, Col8a2, Dnajb11, |
| | | | | | | Dnajb9, Hsp90b1, Hspa5, Mydgf, |
| | | | | | | P3h2, Pdia3, Pdia6, Serpinh1 |
| c5 | 3 | down | GO MITOCHONDRIAL | 0.002 | 0.045 | Cox4i2, Cox6a1, Cycs |
| | | | ELECTRON TRANSPORT | | | |
| | | | CYTOCHROME C TO OXYGEN | | | |
| c5 | 3 | down | GO RESPONSE TO | 0.000 | 0.000 | Atf3, Calr, Dnaja1, Dnajb11, |
| | | | TOPOLOGICALLY INCORRECT | | | Dnajb9, Hsp90b1, Hspa4l, Hspa5, |
| | | | PROTEIN | | | Hspa8, Hspb7, Lmna, Manf, |
| | | | | | | Mydgf, Pdia6, Serpinh1, Srpr, |
| | | | | | | Ssr1, Xbp1 |
| c5 | 3 | up | GO EXTRACELLULAR MATRIX | 0.000 | 0.032 | Ctsk, Ctsl, Gsn, Mmp14, Mmp2, |
| | | | DISASSEMBLY | | | Spp1 |
| c5 | 3 | up | GO OXIDOREDUCTASE | 0.000 | 0.004 | Cygb, Gpx3, Mgst1 |
| | | | ACTIVITY ACTING ON | | | |
| | | | PEROXIDE AS ACCEPTOR | | | |
| c5 | 5 | down | GO ENDOPLASMIC | 0.000 | 0.047 | Arsi, Calr, Col1a1, Col1a2, |
| | | | RETICULUM LUMEN | | | Col3a1, Col5a1, Col6a1, Col6a2, |
| | | | | | | Col8a1, Col8a2, Hsp90b1, Hspa5, |
| | | | | | | P4ha1, Pdia6, Serpinh1, Tmem43 |
| c5 | 5 | down | GO NUCLEOSIDE | 0.000 | 0.011 | Adk, Cox6c, Cycs, Hspa8, Ldha, |
| | | | MONOPHOSPHATE | | | Ndufa1, Ndufs5, Nt5e, Pgm2, |
| | | | METABOLIC PROCESS | | | Uqcr11 |
| c5 | 5 | down | GO RESPONSE TO | 0.000 | 0.001 | Atf4, Atf6, Calr, Dnajb1, Hsp90b1, |
| | | | TOPOLOGICALLY INCORRECT | | | Hspa5, Hspa8, Lmna, Manf, Pdia6, |
| | | | PROTEIN | | | Serpinh1, Thbs4 |
| c5 | 5 | up | GO NEGATIVE REGULATION | 0.000 | 0.047 | Angptl4, Gas6, Ramp2, Serpine1 |
| | | | OF ENDOTHELIAL CELL | | | |
| | | | APOPTOTIC PROCESS | | | |
| c5 | 5 | up | GO RETINA VASCULATURE | 0.000 | 0.029 | Cyp1b1, Nrp1, Pdgfrb, Rhoj |
| | | | DEVELOPMENT IN CAMERA | | | |
| | | | TYPE EYE | | | |
| c5 | 6 | down | GO ACTIVATING | 0.002 | 0.034 | |
| | | | TRANSCRIPTION FACTOR | | | |
| | | | BINDING | | | |
| c5 | 6 | down | GO ACTIVATION OF PROTEIN | 0.006 | 0.044 | |
| | | | KINASE ACTIVITY | | | |
| c5 | 6 | down | GO ADULT BEHAVIOR | 0.004 | 0.015 | App, Dab1, Epha4, Id2, Pcdh17, |
| | | | | | | Prex2, Pum1, Sncg |
| c5 | 6 | down | GO ADULT LOCOMOTORY | 0.000 | 0.006 | App, Dab1, Epha4, Id2, Prex2, |
| | | | BEHAVIOR | | | Pum1, Sncg |
| c5 | 6 | down | GO AGING | 0.000 | 0.005 | Bbc3, Cdkn1a, Cdkn1c, Cx3cl1, |
| | | | | | | Ddit3, Edn1, Fos, Id2, Jun, Jund, |
| | | | | | | Kcne2, Pdcd4, Ppp1r15a, Socs3, |
| | | | | | | Tbx3, Tgfbr2, Timp3, Vcam1 |
| c5 | 6 | down | GO ANTERIOR POSTERIOR | 0.000 | 0.017 | Btg2, Ddit3, Hes1, Hoxa5, Hoxb4, |
| | | | PATTERN SPECIFICATION | | | Hoxc6, Msx1, Nrarp, Pcsk5, Rarg, |
| | | | | | | Sox17, Tbx3, Tcf15 |
| c5 | 6 | down | GO ANTIGEN PROCESSING | 0.004 | 0.033 | |
| | | | AND PRESENTATION OF | | | |
| | | | PEPTIDE ANTIGEN VIA MHC | | | |
| | | | CLASS I | | | |
| c5 | 6 | down | GO ARTERY DEVELOPMENT | 0.006 | 0.048 | |
| c5 | 6 | down | GO ARTERY MORPHOGENESIS | 0.000 | 0.015 | Dll4, Hes1, hey 1, Jag1, Lrp1, |
| | | | | | | Nrp1 |
| c5 | 6 | down | GO ATPASE BINDING | 0.000 | 0.014 | Ar, Bbc3, Dnajb1, Gabarapl2, Rdx, |
| | | | | | | S100a1, Usp25 |
| c5 | 6 | down | GO BODY MORPHOGENESIS | 0.000 | 0.038 | |
| c5 | 6 | down | GO CARDIAC CHAMBER | 0.006 | 0.029 | Adamts1, Cited2, Cyr61, Dll4, |
| | | | MORPHOGENESIS | | | Hes1, hey 1, Id2, Jag1, Sema3c, |
| | | | | | | Tbx3 |
| c5 | 6 | down | GO CARDIAC SEPTUM | 0.000 | 0.009 | Bmp4, Cited2, Cyr61, Hes1, hey 1, |
| | | | DEVELOPMENT | | | Id2, Jag1, Pcsk5, Tbx3 |
| c5 | 6 | down | GO CARDIAC SEPTUM | 0.000 | 0.011 | Cited2, Cyr61, Fzd2, Hes1, hey 1, |
| | | | MORPHOGENESIS | | | Id2, Jag1, Tbx3 |
| c5 | 6 | down | GO CARDIOCYTE | 0.000 | 0.003 | Atg5, Hes1, Jag1, Kdm6b, Mapk3, |
| | | | DIFFERENTIATION | | | Nebl, Sox17, Sox18, Tbx3, Tgfb2 |
| c5 | 6 | down | GO CELLULAR MONOVALENT | 0.020 | 0.049 | |
| | | | INORGANIC CATION | | | |
| | | | HOMEOSTASIS | | | |
| c5 | 6 | down | GO CELLULAR RESPONSE TO | 0.000 | 0.034 | |
| | | | CALCIUM ION | | | |
| c5 | 6 | down | GO CELLULAR RESPONSE TO | 0.000 | 0.000 | B2m, Bst2, Cdk9, Cx3cl1, Cxcl1, |
| | | | CYTOKINE STIMULUS | | | Edn1, Egr1, Gas6, Hfe, Hspa5, |
| | | | | | | Ifitm3, Irf1, Irf2, Irf7, Irf9, Isg15, |
| | | | | | | Klf2, Lifr, Mapk3, Nfkbia, Npnt, |
| | | | | | | Osmr, Plcb1, Psmb10, Psmb8, |
| | | | | | | Psmb9, Rsad2, Sbno2, Socs1, |
| | | | | | | Socs3, Tnfsf12, Ubb, Ubc, Ube2k, |
| | | | | | | Vcam1, Xaf1 |
| c5 | 6 | down | GO CELLULAR RESPONSE TO | 0.000 | 0.009 | Adamts1, Atf3, Atf4, Cdkn1a, |
| | | | EXTRACELLULAR STIMULUS | | | Comt, Fos, Gabarapl2, Gas6, Glul, |
| | | | | | | Hfe, Hspa5, Hspa8, Jun, Ppp1r15a, |
| | | | | | | Rnf152, Sphk1 |
| c5 | 6 | down | GO CELLULAR RESPONSE TO | 0.000 | 0.003 | B2m, Cx3cl1, Edn1, Gbp5, Hfe, |
| | | | INTERFERON GAMMA | | | Irf1, Irf2, Irf7, Irf9, Trim5, Vcam1 |
| c5 | 6 | down | GO CELLULAR RESPONSE TO | 0.000 | 0.012 | Cx3cl1, Edn1, Egr1, Klf2, Mapk3, |
| | | | INTERLEUKIN 1 | | | Plcb1, Psmb9, Rora |
| c5 | 6 | down | GO CELLULAR RESPONSE TO | 0.002 | 0.016 | Arl6ip5, Atf4, Epas1, Fos, Hspa1a, |
| | | | OXIDATIVE STRESS | | | Hspa1b, Kdm6b, Klf2, Klf4, Klf6, |
| | | | | | | Mgst1, Nfe2l1, Pdgfd, Rhob, |
| | | | | | | Sphk1 |
| c5 | 6 | down | GO CELLULAR RESPONSE TO | 0.000 | 0.001 | Atf3, Atf4, Cdkn1a, Comt, |
| | | | STARVATION | | | Gabarapl2, Gas6, Glul, Hfe, |
| | | | | | | Hspa5, Hspa8, Jun, Ppp1r15a, |
| | | | | | | Rnf152, Sphk1 |
| c5 | 6 | down | GO CELLULAR RESPONSE TO | 0.000 | 0.001 | Atf3, Atf4, Dctn1, Ddit3, Dnajb9, |
| | | | TOPOLOGICALLY INCORRECT | | | Hspa5, Pdia6, Ppp1r15a |
| | | | PROTEIN | | | |
| c5 | 6 | down | GO CELL ACTIVATION | 0.000 | 0.014 | Apold1, B2m, Bst2, Cx3cl1, Dll4, |
| | | | | | | Edn1, Egr1, Foxp1, Gas6, Hspb1, |
| | | | | | | Id2, Iif1, Jun, Klf6, Mapk3, Nck1, |
| | | | | | | Plcb1, Plscr1, Psmb10, Rhob, |
| | | | | | | Rora, Rsad2, S100a13, Sbno2, |
| | | | | | | Tgfbr2, Vcam1, Zfp36l1, Zfp36l2 |
| c5 | 6 | down | GO CELL CELL SIGNALING | 0.001 | 0.044 | |
| c5 | 6 | down | GO CELL CHEMOTAXIS | 0.002 | 0.011 | Cx3cl1, Cxcl1, Edn1, Gas6, Nov, |
| | | | | | | Nr4a1, Nrp1, Pdgfb, Tgfb2, Vcam1 |
| c5 | 6 | down | GO CELL CYCLE ARREST | 0.004 | 0.042 | |
| c5 | 6 | down | GO CELL CYCLE G2 M PHASE | 0.004 | 0.014 | Cdkn1a, Dctn1, Dynll1, Plcb1, |
| | | | TRANSITION | | | Ppp1cb, Ubb, Ubc |
| c5 | 6 | down | GO CELL CYCLE PHASE | 0.009 | 0.049 | |
| | | | TRANSITION | | | |
| c5 | 6 | down | GO CELL CYCLE PROCESS | 0.000 | 0.043 | |
| c5 | 6 | down | GO CELL FATE COMMITMENT | 0.003 | 0.013 | Dll4, Epas1, Fgf13, Hes1, Id2, |
| | | | | | | Ifrd1, Jag1, Kdm6b, Klf4, Mcl1, |
| | | | | | | Nrp1, Sox17, Sox18, Tbx3, Tgfb2 |
| c5 | 6 | down | GO CELL MATURATION | 0.002 | 0.021 | App, Cdkn1a, Cdkn1c, Epas1, |
| | | | | | | Hes1, Hoxa5, Id2, Klf2, Maea, |
| | | | | | | Sox18 |
| c5 | 6 | down | GO CELL REDOX | 0.000 | 0.030 | |
| | | | HOMEOSTASIS | | | |
| c5 | 6 | down | GO CHROMATIN | 0.000 | 0.025 | Atrx, Cbx1, Cited2, H1foo, H3f3b, |
| | | | | | | Irf1, Jun, Junb, Jund, Klf4, Plcb1, |
| | | | | | | Ppp1r10, Rarg, Runx2, Sfr1, |
| | | | | | | Smarca2, Sox18 |
| c5 | 6 | down | GO CHROMATIN BINDING | 0.000 | 0.046 | |
| c5 | 6 | down | GO CIRCADIAN RHYTHM | 0.005 | 0.020 | Atf4, Clock, Id1, Id2, Id3, Id4, Jun, |
| | | | | | | Jund, Ppp1cb, Rora |
| c5 | 6 | down | GO CONNECTIVE TISSUE | 0.002 | 0.012 | Arid5b, Crip1, Cyr61, Edn1, Egr1, |
| | | | DEVELOPMENT | | | Hoxa5, Id2, Id4, Mapk3, Msx1, |
| | | | | | | Nov, Pdgfb, Rarg, Snai1, Tgfb2, |
| | | | | | | Tgfbr2, Thra |
| c5 | 6 | down | GO CORE PROMOTER | 0.000 | 0.003 | Atf3, Atf4, Clock, Ddit3, Fos, |
| | | | PROXIMAL REGION DNA | | | Fosb, Hoxa5, Irf1, Jun, Junb, Jund, |
| | | | BINDING | | | Klf13, Klf4, Nr3c1, Rfx1, Skil, |
| | | | | | | Sox18, Tbx3 |
| c5 | 6 | down | GO CYTOKINE MEDIATED | 0.000 | 0.000 | B2m, Bst2, Cx3cl1, Cxcl1, Egr1, |
| | | | SIGNALING PATHWAY | | | Hfe, Ifitm3, Irf1, Irf2, Irf7, Irf9, |
| | | | | | | Isg15, Lifr, Mapk3, Osmr, Plcb1, |
| | | | | | | Psmb10, Psmb8, Psmb9, Rsad2, |
| | | | | | | Socs1, Socs3, Tnfsf12, Ubb, Ubc, |
| | | | | | | Vcam1, Xaf1 |
| c5 | 6 | down | GO DEVELOPMENTAL | 0.007 | 0.040 | |
| | | | MATURATION | | | |
| c5 | 6 | down | GO DNA BIOSYNTHETIC | 0.016 | 0.047 | |
| | | | PROCESS | | | |
| c5 | 6 | down | GO DNA INTEGRITY | 0.013 | 0.044 | |
| | | | CHECKPOINT | | | |
| c5 | 6 | down | GO DNA TEMPLATED | 0.003 | 0.033 | |
| | | | TRANSCRIPTION INITIATION | | | |
| c5 | 6 | down | GO DOUBLE STRANDED DNA | 0.000 | 0.000 | Arid5b, Atf3, Atf4, Cdk9, Clock, |
| | | | BINDING | | | Crip1, Ddit3, Egr1, Fos, Fosb, |
| | | | | | | H3f3b, hey 1, Hoxa5, Irf1, Irf2, |
| | | | | | | Irf7, Jun, Junb, Jund, Klf13, Klf4, |
| | | | | | | Klf6, Lmo2, Msx1, Nfe2l1, Nr3c1, |
| | | | | | | Nsd1, Rarg, Rfx1, Skil, Snai1, |
| | | | | | | Sox13, Sox18, Tbx3, Thra |
| c5 | 6 | down | GO EAR DEVELOPMENT | 0.000 | 0.025 | Edn1, Hes1, Jag1, Mapk3, Msx1, |
| | | | | | | Nrp1, Pls3, Slc9a3r2, Tcf15, Zeb1 |
| c5 | 6 | down | GO EMBRYONIC | 0.000 | 0.005 | Cdkn1c, Col8a1, Cyr61, Dll4, |
| | | | MORPHOGENESIS | | | Dusp1, Edn1, Hes1, Hlx, Hoxa5, |
| | | | | | | Hoxb4, Id2, Irx3, Kdm6b, Klf4, |
| | | | | | | Mapk3, Msx1, Nrp1, Plpp3, Rarg, |
| | | | | | | Snai1, Socs3, Sox17, Sox18, Tbx3, |
| | | | | | | Tgfbr2, Vegfc, Zfp36l1 |
| c5 | 6 | down | GO EMBRYONIC ORGAN | 0.000 | 0.000 | Cdkn1c, Cited2, Cyr61, Ddit3, |
| | | | DEVELOPMENT | | | Dnajb6, Edn1, Epas1, Hes1, hey 1, |
| | | | | | | Hlx, Hoxa5, Hoxb4, Id2, Id3, Junb, |
| | | | | | | Kitl, Mapk3, Msx1, Nrp1, Pcsk5, |
| | | | | | | Pdgfb, Rarg, Snai1, Socs3, Sox17, |
| | | | | | | Sox18, Tbx3, Tgfb2, Tgfbr2, Thra, |
| | | | | | | Zfp36l1 |
| c5 | 6 | down | GO EMBRYONIC ORGAN | 0.000 | 0.021 | Edn1, Hes1, Hlx, Hoxa5, Hoxb4, |
| | | | MORPHOGENESIS | | | Id2, Mapk3, Msx1, Nrp1, Rarg, |
| | | | | | | Runx2, Sox17, Sox18, Tbx3, |
| | | | | | | Tgfbr2, Zeb1 |
| c5 | 6 | down | GO EMBRYONIC PLACENTA | 0.002 | 0.021 | Cdkn1c, Cyr61, Dnajb6, Epas1, |
| | | | DEVELOPMENT | | | Hes1, hey 1, Junb, Pdgfb, Snai1, |
| | | | | | | Socs3, Zfp36l1 |
| c5 | 6 | down | GO EMBRYO DEVELOPMENT | 0.000 | 0.001 | Acvrl1, Ankrd11, Cdkn1c, Cited2, |
| | | | | | | Cyr61, Ddit3, Dll4, Dusp1, Edn1, |
| | | | | | | Efnb1, Egfl8, Epas1, Etl4, Hes1, |
| | | | | | | hey 1, Hlx, Hoxa5, Hoxb4, Id2, |
| | | | | | | Id3, Irx3, Junb, Kdm6b, Kitl, Klf2, |
| | | | | | | Klf4, Mapk3, Msx1, Nrarp, Nrp1, |
| | | | | | | Pcsk5, Pdgfb, Plpp3, Ptprr, Rarg, |
| | | | | | | Skil, Snai1, Socs3, Sox17, Sox18, |
| | | | | | | Tbx3, Tcf15, Tgfb2, Tgfbr2, Thra, |
| | | | | | | Vegfc, Zfp36l1 |
| c5 | 6 | down | GO EMBRYO DEVELOPMENT | 0.000 | 0.002 | Acvrl1, Ankrd11, Cdkn1c, Cyr61, |
| | | | ENDING IN BIRTH OR EGG | | | Edn1, Egfl8, Epas1, Etl4, Hes1, |
| | | | HATCHING | | | hey 1, Hoxa5, Hoxb4, Junb, Klf2, |
| | | | | | | Msx1, Nrarp, Pcsk5, Pdgfb, Ptprr, |
| | | | | | | Rarg, Skil, Snai1, Socs3, Sox17, |
| | | | | | | Sox18, Tbx3, Tcf15, Tgfbr2, |
| | | | | | | Zfp36l1 |
| c5 | 6 | down | GO ENDOCARDIAL CUSHION | 0.000 | 0.035 | |
| | | | DEVELOPMENT | | | |
| c5 | 6 | down | GO ENDOTHELIUM | 0.006 | 0.023 | Acvrl1, Dll4, Gja4, hey 1, Id1, |
| | | | DEVELOPMENT | | | Jag1, Kdm6b, Nrp1, Pecam1, |
| | | | | | | Ppp1r16b, Rapgef2, Rdx, Rhob, |
| | | | | | | Sox17, Sox18 |
| c5 | 6 | down | GO ENZYME LINKED | 0.000 | 0.008 | Acvrl1, Arid5b, Cav2, Cited2, |
| | | | RECEPTOR PROTEIN | | | Csrnp1, Efnb1, Efnb2, Egr1, |
| | | | SIGNALING PATHWAY | | | Epha4, Fermt2, Fos, Gas6, Hfe, |
| | | | | | | Hspb1, Id1, Jun, Lifr, Ltbp4, |
| | | | | | | Mapk3, Mpzl1, Msx1, Nck1, Nrp1, |
| | | | | | | Pdgfb, Pdgfd, Pik3r3, Plcb1, |
| | | | | | | Polr21, Ptprr, Rapgef2, Shoc2, Skil, |
| | | | | | | Tgfb2, Tgfbr2, Tob1, Ubb, Ubc, |
| | | | | | | Vegfc |
| c5 | 6 | down | GO EPITHELIAL CELL | 0.003 | 0.035 | |
| | | | DIFFERENTIATION | | | |
| c5 | 6 | down | GO EPITHELIUM | 0.000 | 0.033 | |
| | | | DEVELOPMENT | | | |
| c5 | 6 | down | GO ERYTHROCYTE | 0.000 | 0.013 | Arid4a, Epas1, Id2, Klf2, Maea, |
| | | | HOMEOSTASIS | | | Nfe2l1, Thra |
| c5 | 6 | down | GO FACE DEVELOPMENT | 0.000 | 0.012 | Ankrd11, Arid5b, Csrnp1, Mapk3, |
| | | | | | | Msx1, Rarg, Tgfb2 |
| c5 | 6 | down | GO FC EPSILON RECEPTOR | 0.000 | 0.001 | Calm1, Fos, Jun, Mapk3, Nfkbia, |
| | | | SIGNALING PATHWAY | | | Psmb10, Psmb8, Psmb9, Ubb, Ubc |
| c5 | 6 | down | GO FC RECEPTOR SIGNALING | 0.011 | 0.031 | |
| | | | PATHWAY | | | |
| c5 | 6 | down | GO G1 DNA DAMAGE | 0.011 | 0.041 | |
| | | | CHECKPOINT | | | |
| c5 | 6 | down | GO GROWTH FACTOR | 0.002 | 0.016 | App, Cd2ap, Pdgfb, Pdgfd, Plscr1, |
| | | | RECEPTOR BINDING | | | Trip6, Vegfc |
| c5 | 6 | down | GO HEART DEVELOPMENT | 0.000 | 0.016 | Adamts1, Cited2, Crip1, Cyr61, |
| | | | | | | Dll4, Edn1, Galnt11, Hes1, |
| | | | | | | Hexim1, hey 1, Id1, Id2, Id3, Jag1, |
| | | | | | | Jun, Kdm6b, Mapk3, Msx1, Nebl, |
| | | | | | | Nrp1, Pcsk5, Pdgfb, Snai1, Sox17, |
| | | | | | | Sox18, Tbx3, Tgfb2, Tgfbr2, |
| | | | | | | Zfp36l1 |
| c5 | 6 | down | GO HEART MORPHOGENESIS | 0.000 | 0.016 | Adamts1, Cited2, Cyr61, Dll4, |
| | | | | | | Hes1, hey 1, Id2, Jag1, Jun, Msx1, |
| | | | | | | Sema3c, Snai1, Sox17, Sox18, |
| | | | | | | Tbx3, Tgfb2 |
| c5 | 6 | down | GO HEPATICOBILIARY | 0.000 | 0.023 | Arf6, Arid5b, Cited2, Hes1, Hfe, |
| | | | SYSTEM DEVELOPMENT | | | Hlx, Jun, Pou2f1, Psmb9, Sox17, |
| | | | | | | Thra |
| c5 | 6 | down | GO HIPPOCAMPUS | 0.004 | 0.025 | Btg2, Fgf13, Id4, Kdm6b |
| | | | DEVELOPMENT | | | |
| c5 | 6 | down | GO IMMUNE RESPONSE | 0.005 | 0.044 | |
| | | | REGULATING CELL SURFACE | | | |
| | | | RECEPTOR SIGNALING | | | |
| | | | PATHWAY | | | |
| c5 | 6 | down | GO IMMUNE SYSTEM | 0.000 | 0.000 | Ace, Arid4a, B2m, Cdkn1c, |
| | | | DEVELOPMENT | | | Cited2, Crip2, Dll4, Egr1, Epas1, |
| | | | | | | Foxp1, Gas6, Herc6, Hes1, Hoxb4, |
| | | | | | | Id2, Irf1, Jag1, Jun, Junb, Kitl, |
| | | | | | | Klf2, Klf4, Klf6, Maea, Mapk3, |
| | | | | | | Mecom, Nfe2l1, Pdgfb, Psmb9, |
| | | | | | | Rsad2, Sbno2, Tgfb2, Tgfbr2, |
| | | | | | | Thra, Vcam1, Zfp36l1, Zfp36l2 |
| c5 | 6 | down | GO INNATE IMMUNE | 0.000 | 0.007 | App, B2m, Bst2, Cx3cl1, Edn1, |
| | | | RESPONSE | | | Egr1, F2rl1, Gbp5, Hfe, Ifitm3, |
| | | | | | | Irf1, Irf2, Irf7, Irf9, Isg15, Nod1, |
| | | | | | | Psmb8, Rsad2, Trim5, Ubb, Ubc, |
| | | | | | | Vcam1, Xaf1 |
| c5 | 6 | down | GO INTERFERON GAMMA | 0.000 | 0.011 | B2m, Hfe, Irf1, Irf2, Irf7, Irf9, |
| | | | MEDIATED SIGNALING | | | Trim5, Vcam1 |
| | | | PATHWAY | | | |
| c5 | 6 | down | GO INTRACELLULAR | 0.004 | 0.035 | |
| | | | RECEPTOR SIGNALING | | | |
| | | | PATHWAY | | | |
| c5 | 6 | down | GO IN UTERO EMBRYONIC | 0.000 | 0.001 | Acvrl1, Ankrd11, Cdkn1c, Cyr61, |
| | | | DEVELOPMENT | | | Edn1, Egfl8, Epas1, Hes1, hey 1, |
| | | | | | | Junb, Klf2, Msx1, Pdgfb, Ptprr, |
| | | | | | | Skil, Snai1, Socs3, Sox17, Sox18, |
| | | | | | | Tbx3, Tgfbr2, Zfp36l1 |
| c5 | 6 | down | GO KINASE BINDING | 0.000 | 0.035 | |
| c5 | 6 | down | GO KINASE INHIBITOR | 0.000 | 0.008 | Cdkn1a, Cdkn1c, Chad, Hexim1, |
| | | | ACTIVITY | | | Hspb1, Nck1, Socs1, Socs3 |
| c5 | 6 | down | GO KINASE REGULATOR | 0.000 | 0.000 | Ccnl1, Cdkn1a, Cdkn1c, Fgf13, |
| | | | ACTIVITY | | | Gas6, Hexim1, Hspbl, Klf4, Nck1, |
| | | | | | | Pik3r3, Socs1, Socs3 |
| c5 | 6 | down | GO LABYRINTHINE LAYER | 0.000 | 0.035 | |
| | | | DEVELOPMENT | | | |
| c5 | 6 | down | GO LEARNING | 0.004 | 0.033 | |
| c5 | 6 | down | GO LEUKOCYTE ACTIVATION | 0.002 | 0.033 | |
| c5 | 6 | down | GO LEUKOCYTE | 0.002 | 0.008 | B2m, Dll4, Egr1, Foxp1, Gas6, |
| | | | DIFFERENTIATION | | | Id2, Irf1, Jun, Junb, Klf6, Rora, |
| | | | | | | Rsad2, Sbno2, Tgfbr2, Vcam1, |
| | | | | | | Zfp36l1, Zfp36l2 |
| c5 | 6 | down | GO MACROMOLECULE | 0.000 | 0.009 | Arid4a, Ash1l, Atrx, Btg2, Fos, |
| | | | METHYLATION | | | Mecom, Nsd1, Pcmtd1, Prdm16, |
| | | | | | | Setd6 |
| c5 | 6 | down | GO MAINTENANCE OF CELL | 0.000 | 0.009 | Epas1, Hes1, Jag1, Klf4, Polr2l, |
| | | | NUMBER | | | Prdm16, Setd6, Tbx3, Zfp3612 |
| c5 | 6 | down | GO MESENCHYMAL CELL | 0.004 | 0.033 | |
| | | | DIFFERENTIATION | | | |
| c5 | 6 | down | GO MESENCHYME | 0.003 | 0.013 | Bmp4, Cited2, Edn1, Edn3, Efnb1, |
| | | | DEVELOPMENT | | | Hes1, hey 1, Hoxa5, Jag1, Kitl, |
| | | | | | | Mapk3, Msx1, Nedd4, Nrp1, |
| | | | | | | Sema3c, Sema3g, Sema7a, Snai1, |
| | | | | | | Tcf15, Tgfb2, Zfp36l1 |
| c5 | 6 | down | GO MESENCHYME | 0.000 | 0.046 | |
| | | | MORPHOGENESIS | | | |
| c5 | 6 | down | GO MESODERM | 0.013 | 0.046 | |
| | | | DEVELOPMENT | | | |
| c5 | 6 | down | GO METANEPHROS | 0.010 | 0.027 | Bmp4, Egr1, Hes1, Id2, Id3, Irx3, |
| | | | DEVELOPMENT | | | Pdgfb |
| c5 | 6 | down | GO METHYLATION | 0.000 | 0.006 | Arid4a, Ash1l, Atrx, Btg2, Comt, |
| | | | | | | Fos, Mat2a, Mecom, Nsd1, |
| | | | | | | Pcmtd1, Prdm16, Setd6 |
| c5 | 6 | down | GO MITOTIC DNA INTEGRITY | 0.012 | 0.049 | |
| | | | CHECKPOINT | | | |
| c5 | 6 | down | GO MORPHOGENESIS OF AN | 0.000 | 0.012 | Acvrl1, Ctsz, Cyr61, Dlg3, Dll4, |
| | | | EPITHELIUM | | | Edn1, Efnb2, Epha4, Hes1, Hoxa5, |
| | | | | | | Hoxb4, Id4, Irx3, Jag1, Klf4, Npnt, |
| | | | | | | Nrarp, Nrp1, Podxl, Rarg, Rhob, |
| | | | | | | Snai1, Socs3, Sox17, Sox18, Tbx3, |
| | | | | | | Tcf15, Tgfb2, Tgfbr2, Vegfc |
| c5 | 6 | down | GO MYELOID CELL | 0.000 | 0.004 | Arid4a, Cdkn1c, Epas1, Foxp1, |
| | | | DIFFERENTIATION | | | Id2, Jun, Junb, Klf2, Maea, Nfe2l1, |
| | | | | | | Sbno2, Tgfbr2, Thra |
| c5 | 6 | down | GO MYELOID CELL | 0.000 | 0.011 | Arid4a, Epas1, Id2, Klf2, Maea, |
| | | | HOMEOSTASIS | | | Nfe2l1, Thra |
| c5 | 6 | down | GO NEGATIVE REGULATION | 0.000 | 0.003 | B2m, Ddit3, hey 1, Hfe, Hspa5, |
| | | | OF BINDING | | | Id1, Id2, Jun, Lhx2, Mapk3, Msx1, |
| | | | | | | Nfkbia, Pdgfb |
| c5 | 6 | down | GO NEGATIVE REGULATION | 0.000 | 0.007 | Ddit3, Egr1, Kremen2, Mcc, |
| | | | OF CANONICAL WNT | | | Psmb10, Psmb8, Psmb9, Sox17, |
| | | | SIGNALING PATHWAY | | | Ubb, Ubc |
| c5 | 6 | down | GO NEGATIVE REGULATION | 0.000 | 0.002 | Cav2, Hspa5, Mecom, Mmrn2, |
| | | | OF CELLULAR RESPONSE TO | | | Prdm16, Skil, Tgfbr2, Tob1, Ubb, |
| | | | GROWTH FACTOR STIMULUS | | | Ubc |
| c5 | 6 | down | GO NEGATIVE REGULATION | 0.000 | 0.027 | Anxa5, Ash1l, Atf3, Cav2, Clock, |
| | | | OF CELL COMMUNICATION | | | Cx3cl1, Ddit3, Dll4, Dnaja1, |
| | | | | | | Dusp1, Dusp26, Edn1, Egr1, |
| | | | | | | Foxp1, Gas6, hey 1, Hspa1a, |
| | | | | | | Hspa1b, Hspa5, Hspb1, Klf4, Mcc, |
| | | | | | | Mcl1, Mecom, Midn, Nck1, |
| | | | | | | Nfkbia, Nov, Nrarp, Nrp1, Pcdh17, |
| | | | | | | Pdcd4, Pim3, Ppp1r15a, Prdm16, |
| | | | | | | Psmb10, Psmb8, Psmb9, Ptprr, |
| | | | | | | Rgs3, Rnf152, S1pr2, Skil, Snai1, |
| | | | | | | Socs1, Socs3, Sox17, Sytl1, |
| | | | | | | Tgfb2, Tgfbr2, Timp3, Tnfsf10, |
| | | | | | | Tob1, Ubb, Ubc |
| c5 | 6 | down | GO NEGATIVE REGULATION | 0.015 | 0.037 | |
| | | | OF CELL CYCLE G1 S PHASE | | | |
| | | | TRANSITION | | | |
| c5 | 6 | down | GO NEGATIVE REGULATION | 0.000 | 0.012 | Anxa5, Atf4, Btg2, Cbx4, Cdkn1a, |
| | | | OF CELL DEATH | | | Cited2, Cx3cl1, Cyr61, Dnaja1, |
| | | | | | | Dusp1, Edn1, Foxp1, Gas6, |
| | | | | | | Hspa1a, Hspa1b, Hspa5, Hspb1, |
| | | | | | | Id1, Ier3, Irf7, Jun, Kitl, Klf4, Lifr, |
| | | | | | | Lims2, Mcl1, Mecom, Msx1, |
| | | | | | | Nck1, Nfkbia, Nov, Nrp1, Pdcd4, |
| | | | | | | Pim3, Ppp1r15a, Rarg, Sgk1, |
| | | | | | | Snai1, Socs3, Son, Tbx3, Tnfsf10, |
| | | | | | | Topors, Ubb, Ubc |
| c5 | 6 | down | GO NEGATIVE REGULATION | 0.000 | 0.013 | Acvrl1, App, Arf6, B2m, Ccdc85b, |
| | | | OF CELL DIFFERENTIATION | | | Ddit3, Dnm3, Epha4, Hes1, Hlx, |
| | | | | | | Hoxa5, Id1, Id2, Id3, Id4, Ifrd1, |
| | | | | | | Irf1, Irx3, Jag1, Klf13, Msx1, |
| | | | | | | Nfkbia, Nov, Nrarp, Nrp1, Pdcd4, |
| | | | | | | Pdgfb, Prdm16, Rapgef2, Rarg, |
| | | | | | | Sema3g, Skil, Snai1, Sox17, |
| | | | | | | Stmn2, Tbx3, Tgfb2, Tob1, Tob2, |
| | | | | | | Zfp36l2 |
| c5 | 6 | down | GO NEGATIVE REGULATION | 0.004 | 0.047 | |
| | | | OF CELL GROWTH | | | |
| c5 | 6 | down | GO NEGATIVE REGULATION | 0.000 | 0.037 | |
| | | | OF DNA BINDING | | | |
| c5 | 6 | down | GO NEGATIVE REGULATION | 0.000 | 0.002 | Bmp4, Hes1, Hlx, Hoxa5, Id2, Irf1, |
| | | | OF HEMOPOIESIS | | | Klf13, Nfkbia, Nrarp, Prdm16, |
| | | | | | | Tob2 |
| c5 | 6 | down | GO NEGATIVE REGULATION | 0.000 | 0.012 | Ash1l, Atf3, Ddit3, Dnaja1, Dusp1, |
| | | | OF INTRACELLULAR SIGNAL | | | Dusp26, Hspa1a, Hspb1, Klf4, |
| | | | TRANSDUCTION | | | Mcl1, Mecom, Nfkbia, Nov, |
| | | | | | | Pdcd4, Ptprr, Rgs3, Rnf152, Snai1, |
| | | | | | | Socs1, Socs3, Tgfb2, Timp3 |
| c5 | 6 | down | GO NEGATIVE REGULATION | 0.002 | 0.012 | Cdkn1a, Cdkn1c, Chad, Dnaja1, |
| | | | OF KINASE ACTIVITY | | | Dusp1, Dusp26, Gadd45b, |
| | | | | | | Hexim1, Hspb1, Midn, Nck1, |
| | | | | | | Pdcd4, Rgs3, Socs1, Socs3, Taf7 |
| c5 | 6 | down | GO NEGATIVE REGULATION | 0.000 | 0.001 | Ash1l, Atf3, Dnaja1, Dusp1, |
| | | | OF MAPK CASCADE | | | Dusp26, F2rl1, Klf4, Mecom, |
| | | | | | | Pdcd4, Ptprr, Rgs3, Timp3 |
| c5 | 6 | down | GO NEGATIVE REGULATION | 0.005 | 0.031 | |
| | | | OF MULTI ORGANISM | | | |
| | | | PROCESS | | | |
| c5 | 6 | down | GO NEGATIVE REGULATION | 0.002 | 0.024 | Ddit3, Id3, Msx1, Nov, Pdgfb, |
| | | | OF MUSCLE CELL | | | Tbx3 |
| | | | DIFFERENTIATION | | | |
| c5 | 6 | down | GO NEGATIVE REGULATION | 0.002 | 0.023 | Ash1l, Atf3, Cdkn1a, Cdkn1c, |
| | | | OF PHOSPHORUS METABOLIC | | | Dlg3, Dnaja1, Dusp1, Dusp26, |
| | | | PROCESS | | | Dynll1, Edn1, Gadd45b, Hexim1, |
| | | | | | | Hspb1, Jun, Klf4, Mecom, Midn, |
| | | | | | | Nck1, Pdcd4, Pdgfb, Plpp3, |
| | | | | | | Ppp1r15a, Ppp1r16b, Ptprr, Rgs3, |
| | | | | | | Socs1, Socs3, Taf7, Tgfb2, Timp3 |
| c5 | 6 | down | GO NEGATIVE REGULATION | 0.000 | 0.017 | Ash1l, Atf3, Cdkn1a, Cdkn1c, |
| | | | OF PHOSPHORYLATION | | | Dnaja1, Dusp1, Dusp26, Dynll1, |
| | | | | | | Gadd45b, Hexim1, Hspb1, Jun, |
| | | | | | | Klf4, Mecom, Midn, Nck1, Pdcd4, |
| | | | | | | Plpp3, Ppp1r15a, Ptprr, Rgs3, |
| | | | | | | Socs1, Socs3, Taf7, Timp3 |
| c5 | 6 | down | GO NEGATIVE REGULATION | 0.004 | 0.013 | Atg5, Dnaja1, Hfe, Hspa1a, |
| | | | OF PROTEIN MODIFICATION | | | Hspa1b, Isg15, Psmb10, Psmb8, |
| | | | BY SMALL PROTEIN | | | Psmb9, Ubb, Ubc |
| | | | CONJUGATION OR REMOVAL | | | |
| c5 | 6 | down | GO NEGATIVE REGULATION | 0.000 | 0.004 | Ash1l, Atf3, Cdkn1a, Cdkn1c, |
| | | | OF PROTEIN MODIFICATION | | | Dlg3, Dnaja1, Dusp1, Dusp26, |
| | | | PROCESS | | | Gadd45b, Hexim1, Hfe, Hspa1a, |
| | | | | | | Hspa1b, Hspb1, Isg15, Jun, Klf4, |
| | | | | | | Mecom, Nck1, Pdcd4, Plpp3, |
| | | | | | | Ppp1r15a, Ppp1r16b, Psmb10, |
| | | | | | | Psmb8, Psmb9, Ptprr, Rgs3, Socs1, |
| | | | | | | Socs3, Taf7, Tgfb2, Timp3, Ubb, |
| | | | | | | Ubc |
| c5 | 6 | down | GO NEGATIVE REGULATION | 0.009 | 0.034 | |
| | | | OF PROTEIN SERINE | | | |
| | | | THREONINE KINASE | | | |
| | | | ACTIVITY | | | |
| c5 | 6 | down | GO NEGATIVE REGULATION | 0.003 | 0.048 | |
| | | | OF REPRODUCTIVE PROCESS | | | |
| c5 | 6 | down | GO NEGATIVE REGULATION | 0.006 | 0.037 | |
| | | | OF SEQUENCE SPECIFIC DNA | | | |
| | | | BINDING TRANSCRIPTION | | | |
| | | | FACTOR ACTIVITY | | | |
| c5 | 6 | down | GO NEGATIVE REGULATION | 0.000 | 0.000 | Arid5b, Atf3, Atrx, Btg2, Cbx4, |
| | | | OF TRANSCRIPTION FROM | | | Cdkn1c, Cited2, Ctbp2, Ddit3, |
| | | | RNA POLYMERASE II | | | Ddx5, Dll4, Dusp26, Edn1, Egr1, |
| | | | PROMOTER | | | Fosb, Hes1, Hexim1, hey 1, |
| | | | | | | Hoxb4, Id1, Id2, Id3, Id4, Irf2, |
| | | | | | | Irf2bpl, Irf7, Jun, Junb, Jund, |
| | | | | | | Klf11, Klf4, Msx1, Nck1, Nrarp, |
| | | | | | | Nsd1, Ppp1r15a, Prdm16, Pth, |
| | | | | | | Rarg, S100a1, Skil, Smarca2, |
| | | | | | | Snai1, Sox17, Sox18, Taf7, Tbx3, |
| | | | | | | Thra, Ubb, Ubc, Zbtb20, Zfp36 |
| c5 | 6 | down | GO NEGATIVE REGULATION | 0.000 | 0.004 | Cdkn1a, Cdkn1c, Dnaja1, Dusp1, |
| | | | OF TRANSFERASE ACTIVITY | | | Dusp26, Gadd45b, Hexim1, |
| | | | | | | Hspb1, Midn, Nck1, Pdcd4, |
| | | | | | | Psmb10, Psmb8, Psmb9, Rgs3, |
| | | | | | | Socs1, Socs3, Taf7, Ubb, Ubc |
| c5 | 6 | down | GO NEGATIVE REGULATION | 0.000 | 0.003 | Cav2, Hspa5, Mecom, Prdm16, |
| | | | OF TRANSFORMING GROWTH | | | Skil, Skor1, Tgfbr2, Ubb, Ubc |
| | | | FACTOR BETA RECEPTOR | | | |
| | | | SIGNALING PATHWAY | | | |
| c5 | 6 | down | GO NEGATIVE REGULATION | 0.000 | 0.000 | Cav2, Hspa5, Mecom, Nov, |
| | | | OF TRANSMEMBRANE | | | Prdm16, Skil, Tgfbr2, Tob1, Ubb, |
| | | | RECEPTOR PROTEIN SERINE | | | Ubc |
| | | | THREONINE KINASE | | | |
| | | | SIGNALING PATHWAY | | | |
| c5 | 6 | down | GO NEGATIVE REGULATION | 0.000 | 0.013 | Bst2, Ifitm3, Isg15, Jun, Plscr1, |
| | | | OF VIRAL PROCESS | | | Rsad2, Trim5 |
| c5 | 6 | down | GO NEGATIVE REGULATION | 0.009 | 0.049 | |
| | | | OF WNT SIGNALING | | | |
| | | | PATHWAY | | | |
| c5 | 6 | down | GO NEURON | 0.000 | 0.041 | |
| | | | DIFFERENTIATION | | | |
| c5 | 6 | down | GO NEURON FATE | 0.000 | 0.024 | Bmp4, Dll4, Hes1, Id2, Tgfb2 |
| | | | COMMITMENT | | | |
| c5 | 6 | down | GO NOTCH SIGNALING | 0.002 | 0.020 | App, Chac1, Dll4, Galnt11, Hes1, |
| | | | PATHWAY | | | hey 1, Jag1, Nrarp, Snai1, Tgfbr2, |
| | | | | | | Ubb, Ubc |
| c5 | 6 | down | GO NUCLEAR BODY | 0.002 | 0.014 | Atrx, Cbx4, Ccnl1, Cdk9, Epas1, |
| | | | | | | Mecom, Plcb1, Sap18, Skil, Son, |
| | | | | | | Srrm1, Srrm2, Srsf2, Tdp2, Topors |
| c5 | 6 | down | GO NUCLEAR CHROMATIN | 0.000 | 0.001 | Atrx, Cbx1, Cited2, H3f3b, Irf1, |
| | | | | | | Jun, Junb, Jund, Klf4, Plcb1, Rarg, |
| | | | | | | Runx2, Sfr1, Smarca2, Sox18 |
| c5 | 6 | down | GO NUCLEAR CHROMOSOME | 0.000 | 0.004 | Atrx, Cbx1, Cited2, H3f3b, |
| | | | | | | Hormad1, Irf1, Jun, Junb, Jund, |
| | | | | | | Klf4, Plcb1, Ppp1cb, Ppp1r10, |
| | | | | | | Rarg, Sfr1, Smarca2, Smchd1, |
| | | | | | | Sox18, Ssb |
| c5 | 6 | down | GO NUCLEAR IMPORT | 0.006 | 0.013 | Hes1, Jun, Mapk3, Nfkbia, |
| | | | | | | Ppp1r10, Rere, Tgfb2, Tob1 |
| c5 | 6 | down | GO NUCLEAR SPECK | 0.002 | 0.008 | Cbx4, Ccnl1, Epas1, Mecom, |
| | | | | | | Plcb1, Sap18, Son, Srrm1, Srrm2, |
| | | | | | | Srsf2, Topors |
| c5 | 6 | down | GO NUCLEIC ACID BINDING | 0.000 | 0.000 | Arid4a, Arid5b, Atf3, Atf4, Btg2, |
| | | | TRANSCRIPTION FACTOR | | | Cited2, Clock, Csrnp1, Ddit3, |
| | | | ACTIVITY | | | Ebf1, Egr1, Epas1, Fos, Fosb, |
| | | | | | | Foxp1, Gpbp1, Hes1, hey 1, |
| | | | | | | Hoxa5, Hoxb4, Id1, Id3, Irf1, Irf2, |
| | | | | | | Irf7, Irf9, Jun, Junb, Jund, Klf11, |
| | | | | | | Klf13, Klf2, Klf4, Klf6, Klf7, |
| | | | | | | Lmo2, Mecom, Mllt10, Msx1, |
| | | | | | | Nfe2l1, Nr3c1, Nr4a1, Plscr1, |
| | | | | | | Rarg, Rfx1, Skil, Snai1, Sox13, |
| | | | | | | Sox17, Sox18, Taf7, Tbx3, Tcf15, |
| | | | | | | Thra, Tulp4, Zbtb20, Zfp36l1, |
| | | | | | | Zfp36l2 |
| c5 | 6 | down | GO PALATE DEVELOPMENT | 0.000 | 0.002 | Arid5b, Csrnp1, Fzd2, Msx1, |
| | | | | | | Prdm16, Snai1, Tbx3, Tgfbr2 |
| c5 | 6 | down | GO POSITIVE REGULATION OF | 0.008 | 0.026 | Atf3, Bbc3, Ddit3, Dynll1, Mcl1, |
| | | | APOPTOTIC SIGNALING | | | Msx1, Nck1, Pias4, Skil, Tgfb2, |
| | | | PATHWAY | | | Timp3, Tnfsf10, Tnfsf12, Ubb |
| c5 | 6 | down | GO POSITIVE REGULATION OF | 0.010 | 0.037 | |
| | | | CANONICAL WNT SIGNALING | | | |
| | | | PATHWAY | | | |
| c5 | 6 | down | GO POSITIVE REGULATION OF | 0.000 | 0.005 | App, Atrx, Btg2, Ccnl1, Cdk9, |
| | | | CELL CYCLE | | | Cdkn1a, Cit, Cited2, Edn1, Hes1, |
| | | | | | | Id2, Msx1, Pdgfb, Plcb1, Rdx, |
| | | | | | | Sphk1, Tbx3, Tgfb2, Ubb, Ubc |
| c5 | 6 | down | GO POSITIVE REGULATION OF | 0.000 | 0.011 | Btg2, Cdkn1a, Cnot6, Id2, Ubb, |
| | | | CELL CYCLE ARREST | | | Ubc |
| c5 | 6 | down | GO POSITIVE REGULATION OF | 0.000 | 0.006 | App, Atrx, Btg2, Cdkn1a, Cit, |
| | | | CELL CYCLE PROCESS | | | Edn1, Id2, Msx1, Pcid2, Pdgfb, |
| | | | | | | Plcb1, Rdx, Ubb, Ubc |
| c5 | 6 | down | GO POSITIVE REGULATION OF | 0.000 | 0.017 | Anxa5, Arhgef6, Arl6ip5, Atf3, |
| | | | CELL DEATH | | | Atf4, Bbc3, Btg1, Cdk19, Cdkn1a, |
| | | | | | | Cyr61, Ddit3, Dnaja1, Dusp1, |
| | | | | | | Dynll1, Gadd45b, Gadd45g, |
| | | | | | | Hoxa5, Id3, Jun, Klf11, Map3k11, |
| | | | | | | Mcl1, Msx1, Nck1, Nod1, Pdcd4, |
| | | | | | | Plekhg2, Ppp1r15a, Rapgef2, Rarg, |
| | | | | | | Rhob, Sap18, Skil, Tgfb2, Timp3, |
| | | | | | | Tnfsf10, Tnfsf12, Ubb, Ubc |
| c5 | 6 | down | GO POSITIVE REGULATION OF | 0.000 | 0.025 | Acvrl1, Adamts1, Btg1, Cx3cl1, |
| | | | CELL DIFFERENTIATION | | | Cyr61, Edn1, Efnb2, Epha4, Fos, |
| | | | | | | Gas6, Hes1, Hlx, Hoxa5, Hoxb4, |
| | | | | | | Id2, Id4, Irx3, Isg15, Jag1, Jun, |
| | | | | | | Junb, Jund, Kitl, Neu1, Npnt, |
| | | | | | | Nrp1, Plcb1, Prdm16, Rapgef2, |
| | | | | | | S1pr2, Sgk1, Skil, Snai1, Socs3, |
| | | | | | | Sox17, Stmn2, Tgfb2, Tgfbr2, |
| | | | | | | Thra, Vegfc |
| c5 | 6 | down | GO POSITIVE REGULATION OF | 0.000 | 0.014 | Acvrl1, Aggf1, Atf3, Cav2, Cd81, |
| | | | CELL PROLIFERATION | | | Cdkn1a, Crip2, Cx3cl1, Cyr61, |
| | | | | | | Dll4, Edn1, Efnb1, Egr1, Emp2, |
| | | | | | | Foxp1, Gas6, Glul, Hes1, Hlx, Id1, |
| | | | | | | Id2, Id4, Jun, Kitl, Lifr, Nck1, |
| | | | | | | Nr4a1, Nrarp, Nrp1, Osmr, Pdgfb, |
| | | | | | | Pdgfd, Ppp1r16b, Rarg, S100a13, |
| | | | | | | S1pr2, Sertad1, Tbx3, Tgfb2, |
| | | | | | | Tgfbr2, Tnfsf12, Vcam1, Vegfc |
| c5 | 6 | down | GO POSITIVE REGULATION OF | 0.000 | 0.035 | |
| | | | EXTRINSIC APOPTOTIC | | | |
| | | | SIGNALING PATHWAY | | | |
| c5 | 6 | down | GO POSITIVE REGULATION OF | 0.000 | 0.017 | B2m, Cd81, Cdkn1a, Cx3cl1, |
| | | | IMMUNE SYSTEM PROCESS | | | Cxcl1, Efnb1, Efnb2, Fos, Gas6, |
| | | | | | | Hes1, Hlx, Hoxa5, Hspa1a, |
| | | | | | | Hspa1b, Id2, Irf7, Isg15, Jag1, Jun, |
| | | | | | | Kitl, Mapk3, Nck1, Nfkbia, Nod1, |
| | | | | | | Pcid2, Pdgfd, Plscrl, Psmb10, |
| | | | | | | Psmb8, Psmb9, Pum1, Rsad2, |
| | | | | | | Tap2, Tgfb2, Tgfbr2, Ubb, Ubc, |
| | | | | | | Vcam1, Vegfc |
| c5 | 6 | down | GO POSITIVE REGULATION OF | 0.001 | 0.046 | |
| | | | INTRACELLULAR SIGNAL | | | |
| | | | TRANSDUCTION | | | |
| c5 | 6 | down | GO POSITIVE REGULATION OF | 0.000 | 0.004 | Ace, Ccdc88a, Ccnl1, Cd81, |
| | | | KINASE ACTIVITY | | | Cdkn1a, Cyr61, Dstyk, Edn1, |
| | | | | | | Emp2, Epha4, Gadd45b, Gadd45g, |
| | | | | | | Gas6, Hspa5, Kitl, Map3k11, |
| | | | | | | Map4k3, Mapk3, Nod1, Pdgfb, |
| | | | | | | Pdgfd, Rapgef2, S1pr2, Tgfb2, |
| | | | | | | Tgfbr2, Ubb, Ubc |
| c5 | 6 | down | GO POSITIVE REGULATION OF | 0.000 | 0.026 | Arl6ip5, Cav2, Cd81, Cx3cl1, |
| | | | MAPK CASCADE | | | Dstyk, Edn1, Epha4, Gadd45b, |
| | | | | | | Gadd45g, Gas6, Jun, Kitl, |
| | | | | | | Map3k11, Map4k3, Mapk3, Nod1, |
| | | | | | | Npnt, Nrp1, Pdgfb, Pdgfd, Plcb1, |
| | | | | | | Rapgef2, S1pr2, Tgfb2, Ubb, Ubc |
| c5 | 6 | down | GO POSITIVE REGULATION OF | 0.000 | 0.040 | |
| | | | MRNA METABOLIC PROCESS | | | |
| c5 | 6 | down | GO POSITIVE REGULATION OF | 0.000 | 0.033 | |
| | | | NEURON APOPTOTIC | | | |
| | | | PROCESS | | | |
| c5 | 6 | down | GO POSITIVE REGULATION OF | 0.011 | 0.032 | |
| | | | NEURON DEATH | | | |
| c5 | 6 | down | GO POSITIVE REGULATION OF | 0.002 | 0.001 | Clock, Hspa1a, Hspa1b, Nfkbia, |
| | | | NF KAPPAB TRANSCRIPTION | | | Nod1, Sphk1, Ubb, Ubc |
| | | | FACTOR ACTIVITY | | | |
| c5 | 6 | down | GO POSITIVE REGULATION OF | 0.000 | 0.041 | |
| | | | PHOSPHORUS METABOLIC | | | |
| | | | PROCESS | | | |
| c5 | 6 | down | GO POSITIVE REGULATION OF | 0.000 | 0.033 | |
| | | | PROTEIN MODIFICATION | | | |
| | | | PROCESS | | | |
| c5 | 6 | down | GO POSITIVE REGULATION OF | 0.000 | 0.014 | Ccnl1, Cd81, Edn1, Epha4, |
| | | | PROTEIN SERINE THREONINE | | | Gadd45b, Gadd45g, Kitl, |
| | | | KINASE ACTIVITY | | | Map3k11, Map4k3, Mapk3, Nod1, |
| | | | | | | Pdgfb, Pdgfd, Rapgef2, S1pr2, |
| | | | | | | Ubb, Ubc |
| c5 | 6 | down | GO POSITIVE REGULATION OF | 0.000 | 0.006 | Arid5b, Clock, Hspa1a, Hspa1b, |
| | | | SEQUENCE SPECIFIC DNA | | | Nfkbia, Nod1, Plpp3, Sphk1, Ubb, |
| | | | BINDING TRANSCRIPTION | | | Ubc |
| | | | FACTOR ACTIVITY | | | |
| c5 | 6 | down | GO POSITIVE REGULATION OF | 0.000 | 0.026 | Bmp4, Cx3cl1, Edn1, Foxp1, Id2, |
| | | | SMOOTH MUSCLE CELL | | | Jun, Pdgfb, Pdgfd, Tgfbr2 |
| | | | PROLIFERATION | | | |
| c5 | 6 | down | GO POSITIVE REGULATION OF | 0.000 | 0.025 | Dab2, Hoxb4, Snai1, Tgfb2, |
| | | | STEM CELL | | | Tgfbr2 |
| | | | DIFFERENTIATION | | | |
| c5 | 6 | down | GO POSITIVE REGULATION OF | 0.000 | 0.006 | Arl6ip5, Edn1, Epha4, F2rl1, |
| | | | STRESS ACTIVATED PROTEIN | | | Gadd45b, Gadd45g, Map3k11, |
| | | | KINASE SIGNALING CASCADE | | | Nod1, Plcb1, Tgfb2 |
| c5 | 6 | down | GO POSITIVE REGULATION OF | 0.000 | 0.000 | Acvrl1, App, Arid4a, Ash1l, Atf3, |
| | | | TRANSCRIPTION FROM RNA | | | Atf4, Atrx, Ccnl1, Cd81, Cdk9, |
| | | | POLYMERASE II PROMOTER | | | Cited2, Clock, Csrnp1, Ctbp2, |
| | | | | | | Cyr61, Ddit3, Ddx5, Ebf1, Edn1, |
| | | | | | | Egr1, Epas1, Fos, Fosb, Hes1, |
| | | | | | | hey 1, Hoxa5, Hoxb4, Hspa5, Id2, |
| | | | | | | Id4, Iif1, Irf2, Irf2bpl, Irf7, Jag1, |
| | | | | | | Jun, Junb, Jund, Kdm6b, Klf13, |
| | | | | | | Klf2, Klf4, Klf6, Lmo2, Mapk3, |
| | | | | | | Mllt10, Msx1, Nck1, Nfe2l1, |
| | | | | | | Nfkbia, Npnt, Nr3c1, Nr4a1, |
| | | | | | | Pdgfb, Plscrl, Rarg, Sbno2, |
| | | | | | | Sertad1, Smarca2, Sox17, Sox18, |
| | | | | | | Taf1, Taf7, Thra, Ubb, Ubc |
| c5 | 6 | down | GO POSITIVE REGULATION OF | 0.000 | 0.001 | Ace, Ccdc88a, Ccnl1, Cd81, |
| | | | TRANSFERASE ACTIVITY | | | Cdkn1a, Cyr61, Dstyk, Edn1, |
| | | | | | | Emp2, Epha4, Gadd45b, Gadd45g, |
| | | | | | | Gas6, Hspa5, Kitl, Klf4, Map3k11, |
| | | | | | | Map4k3, Mapk3, Nod1, Pdgfb, |
| | | | | | | Pdgfd, Psmb10, Psmb8, Psmb9, |
| | | | | | | Rapgef2, S1pr2, Tgfb2, Tgfbr2, |
| | | | | | | Topors, Ubb, Ubc |
| c5 | 6 | down | GO POSITIVE REGULATION OF | 0.016 | 0.038 | |
| | | | TRANSMEMBRANE RECEPTOR | | | |
| | | | PROTEIN SERINE THREONINE | | | |
| | | | KINASE SIGNALING | | | |
| | | | PATHWAY | | | |
| c5 | 6 | down | GO POSTTRANSCRIPTIONAL | 0.003 | 0.040 | |
| | | | REGULATION OF GENE | | | |
| | | | EXPRESSION | | | |
| c5 | 6 | down | GO PROTEASOMAL PROTEIN | 0.003 | 0.049 | |
| | | | CATABOLIC PROCESS | | | |
| c5 | 6 | down | GO PROTEIN ALKYLATION | 0.004 | 0.019 | Arid4a, Ash1l, Btg2, Mecom, |
| | | | | | | Nsd1, Pcmtd1, Prdm16, Setd6 |
| c5 | 6 | down | GO PROTEIN LOCALIZATION | 0.000 | 0.002 | Arl2bp, Dnajb6, Hes1, Jun, |
| | | | TO NUCLEUS | | | Mapk3, Msx1, Nfkbia, Ppp1r10, |
| | | | | | | Sun1, Syne2, Tgfb2, Tob1, Topors |
| c5 | 6 | down | GO PROTEIN LOCALIZATION | 0.000 | 0.001 | Arf6, Arl2bp, Atrx, Bbc3, Cd81, |
| | | | TO ORGANELLE | | | Dnaja1, Dnajb6, Hes1, Hspa5, |
| | | | | | | Hspa8, Jun, Mapk3, Msx1, Nfkbia, |
| | | | | | | Nrp1, Ppp1r10, Ppp1r15a, Ssb, |
| | | | | | | Sun1, Syne2, Tgfb2, Tob1, Topors, |
| | | | | | | Tulp4 |
| c5 | 6 | down | GO PROTEIN SECRETION | 0.008 | 0.028 | Foxp1, Pcsk5, Pdia4, S100a13, |
| | | | | | | Sncg, Ube2q1 |
| c5 | 6 | down | GO PROTEIN TYROSINE | 0.016 | 0.045 | |
| | | | PHOSPHATASE ACTIVITY | | | |
| c5 | 6 | down | GO REGIONALIZATION | 0.000 | 0.020 | Btg2, Cited2, Ddit3, Dll4, Edn1, |
| | | | | | | Hes1, Hoxa5, Hoxb4, Hoxc6, Irx3, |
| | | | | | | Msx1, Nrarp, Nrp1, Pcsk5, Rarg, |
| | | | | | | Snai1, Sox17, Tbx3, Tcf15, Tra2b |
| c5 | 6 | down | GO REGULATION OF | 0.000 | 0.016 | B2m, Hfe, Hlx, Irf1, Irf7, Prkcz, |
| | | | ADAPTIVE IMMUNE | | | Rsad2 |
| | | | RESPONSE | | | |
| c5 | 6 | down | GO REGULATION OF ATPASE | 0.002 | 0.025 | Ahsa1, Dnajb1, Dnajb6, |
| | | | ACTIVITY | | | Gabarapl2, Plscr1 |
| c5 | 6 | down | GO REGULATION OF BINDING | 0.000 | 0.001 | App, B2m, Cdk9, Ctbp2, Ddit3, |
| | | | | | | Epha4, Hes1, hey 1, Hfe, Hspa5, |
| | | | | | | Id1, Id2, Jun, Klf4, Mapk3, Msx1, |
| | | | | | | Nfkbia, Nrp1, Nsd1, Pdgfb, Prmt7, |
| | | | | | | Rapgef2, Tgfb2 |
| c5 | 6 | down | GO REGULATION OF BMP | 0.000 | 0.004 | Acvrl1, Cyr61, Hes1, Msx1, Skor1, |
| | | | SIGNALING PATHWAY | | | Tob1 |
| c5 | 6 | down | GO REGULATION OF | 0.002 | 0.012 | Ddit3, Egr1, Kremen2, Mcc, |
| | | | CANONICAL WNT SIGNALING | | | Nrarp, Psmb10, Psmb8, Psmb9, |
| | | | PATHWAY | | | Sox17, Ubb, Ubc |
| c5 | 6 | down | GO REGULATION OF | 0.000 | 0.001 | Acvrl1, Cav2, Cdkn1c, Cited2, |
| | | | CELLULAR RESPONSE TO | | | Cyr61, Dstyk, Hes1, Hspa5, Lrit3, |
| | | | GROWTH FACTOR STIMULUS | | | Ltbp4, Mecom, Msx1, Npnt, |
| | | | | | | Prdm16, Skil, Tgfbr2, Tob1, Ubb, |
| | | | | | | Ubc, Vegfc |
| c5 | 6 | down | GO REGULATION OF | 0.000 | 0.001 | Arl6ip5, Atf4, Bbc3, Cdk9, Ddit3, |
| | | | CELLULAR RESPONSE TO | | | Ddx5, Dnaja1, Dnajb1, Edn1, |
| | | | STRESS | | | Epha4, Fbln5, Gadd45b, Gadd45g, |
| | | | | | | Hspa1a, Hspa1b, Hspa5, Hspa8, |
| | | | | | | Hspb1, Ier3, Klf4, Map3k11, |
| | | | | | | Mapk3, Mcl1, Mecom, Msx1, |
| | | | | | | Nck1, Nod1, Nuak1, Pdcd4, Plcb1, |
| | | | | | | Ppp1r15a, Ptges3, Skil, Snai1, |
| | | | | | | Tgfb2, Usp25, Ypel3 |
| c5 | 6 | down | GO REGULATION OF | 0.000 | 0.001 | Cav2, Cdkn1c, Cited2, Hspa5, |
| | | | CELLULAR RESPONSE TO | | | Ltbp4, Mecom, Npnt, Prdm16, |
| | | | TRANSFORMING GROWTH | | | Skil, Skor1, Tgfbr2, Ubb, Ubc |
| | | | FACTOR BETA STIMULUS | | | |
| c5 | 6 | down | GO REGULATION OF CELL | 0.000 | 0.000 | App, Atrx, Btg2, Cables2, Cav2, |
| | | | CYCLE | | | Ccnl1, Cdk19, Cdk9, Cdkn1a, |
| | | | | | | Cdkn1c, Cit, Cited2, Ddit3, Dusp1, |
| | | | | | | Edn1, Gadd45b, Gadd45g, Gas6, |
| | | | | | | Hes1, Hexim1, Hspa8, Id2, Id3, |
| | | | | | | Irf1, Jun, Junb, Jund, Maea, |
| | | | | | | Mecom, Msx1, Nr4a1, Pdcd4, |
| | | | | | | Pdgfb, Pim3, Plcb1, Ppp1r15a, |
| | | | | | | Psmb10, Psmb8, Psmb9, Pum1, |
| | | | | | | Rdx, Rhob, Sertad1, Sgk1, Skil, |
| | | | | | | Son, Tbx3, Tgfb2, Ubb, Ubc |
| c5 | 6 | down | GO REGULATION OF CELL | 0.000 | 0.004 | Btg2, Cdkn1a, Cnot6, Id2, Plcb1, |
| | | | CYCLE G1 S PHASE | | | Rdx, Ubb, Ubc |
| | | | TRANSITION | | | |
| c5 | 6 | down | GO REGULATION OF CELL | 0.000 | 0.010 | App, Btg2, Cdkn1a, Dusp1, Id2, |
| | | | CYCLE PHASE TRANSITION | | | Pcid2, Plcb1, Psmb10, Psmb8, |
| | | | | | | Psmb9, Rdx, Ubb, Ubc |
| c5 | 6 | down | GO REGULATION OF CELL | 0.002 | 0.046 | |
| | | | CYCLE PROCESS | | | |
| c5 | 6 | down | GO REGULATION OF CELL | 0.004 | 0.050 | |
| | | | DIVISION | | | |
| c5 | 6 | down | GO REGULATION OF | 0.000 | 0.034 | |
| | | | CYTOKINE PRODUCTION | | | |
| c5 | 6 | down | GO REGULATION OF | 0.020 | 0.044 | |
| | | | DENDRITE MORPHOGENESIS | | | |
| c5 | 6 | down | GO REGULATION OF DNA | 0.000 | 0.000 | Ddit3, Hes1, hey 1, Id1, Id2, Jun, |
| | | | BINDING | | | Klf4, Lhx2, Msx1, Nfkbia, Nsd1 |
| c5 | 6 | down | GO REGULATION OF DNA | 0.000 | 0.008 | Acvrl1, Cdkn1a, Dusp1, Fgfr4, |
| | | | BIOSYNTHETIC PROCESS | | | Klf4, Mapk3, Pdgfb, Prkd2 |
| c5 | 6 | down | GO REGULATION OF DNA | 0.002 | 0.040 | |
| | | | METABOLIC PROCESS | | | |
| c5 | 6 | down | GO REGULATION OF DNA | 0.000 | 0.029 | Acvrl1, Atrx, Bmp4, Ccdc88a, Id3, |
| | | | REPLICATION | | | Jun, Kitl, Klf4, Mapk3, Pdgfb |
| c5 | 6 | down | GO REGULATION OF DNA | 0.000 | 0.001 | Atf3, Atf4, Cited2, Ddit3, Egr1, |
| | | | TEMPLATED TRANSCRIPTION | | | Epas1, Hspa5, Jun, Klf2, Nck1, |
| | | | IN RESPONSE TO STRESS | | | Nedd4, Ppp1r15a, Ubb, Ubc |
| c5 | 6 | down | GO REGULATION OF | 0.004 | 0.028 | Grhl3, Hes1, Klf4, Nrarp, Plcb1, |
| | | | EMBRYONIC DEVELOPMENT | | | Snai1, Sox17 |
| c5 | 6 | down | GO REGULATION OF | 0.000 | 0.011 | Acvrl1, Apold1, Btg1, Clock, |
| | | | EPITHELIAL CELL | | | Hes1, Id1, Kitl, S1pr2, Tbx3 |
| | | | DIFFERENTIATION | | | |
| c5 | 6 | down | GO REGULATION OF | 0.000 | 0.042 | |
| | | | EPITHELIAL CELL | | | |
| | | | PROLIFERATION | | | |
| c5 | 6 | down | GO REGULATION OF | 0.004 | 0.031 | |
| | | | EPITHELIAL TO | | | |
| | | | MESENCHYMAL TRANSITION | | | |
| c5 | 6 | down | GO REGULATION OF ERK1 | 0.000 | 0.002 | Atf3, Atp6ap1, Cx3cl1, Cyr61, |
| | | | AND ERK2 CASCADE | | | Dstyk, Dusp1, Dusp26, F2rl1, |
| | | | | | | Gas6, Jun, Klf4, Mapk3, Nod1, |
| | | | | | | Npnt, Nrp1, Pdgfb, Pdgfd, Ptprr, |
| | | | | | | Rapgef2, Timp3 |
| c5 | 6 | down | GO REGULATION OF | 0.000 | 0.010 | Ar, Atf3, Cx3cl1, Hspa1a, Hspa1b, |
| | | | EXTRINSIC APOPTOTIC | | | Mcl1, Nrp1, Skil, Tgfb2, Timp3, |
| | | | SIGNALING PATHWAY | | | Tnfsf10, Tnfsf12 |
| c5 | 6 | down | GO REGULATION OF FAT | 0.002 | 0.003 | Ccdc85b, Ddit3, Hes1, Id2, Id4, |
| | | | CELL DIFFERENTIATION | | | Jag1, Prdm16, Rora |
| c5 | 6 | down | GO REGULATION OF GLIAL | 0.000 | 0.012 | Dab1, Epha4, Hes1, Id2, Id4 |
| | | | CELL DIFFERENTIATION | | | |
| c5 | 6 | down | GO REGULATION OF | 0.004 | 0.015 | Dab1, Epha4, Hes1, Id2, Id4 |
| | | | GLIOGENESIS | | | |
| c5 | 6 | down | GO REGULATION OF | 0.000 | 0.008 | Ace, Fos, Foxp1, Gas6, Hes1, Hlx, |
| | | | HEMOPOIESIS | | | Hoxa5, Id2, Irf1, Irf7, Isg15, Jag1, |
| | | | | | | Jun, Kitl, Klf13, Nfkbia, Nrarp, |
| | | | | | | Pcid2, Prdm16, Rarg, Sox13, |
| | | | | | | Tgfbr2, Tob2 |
| c5 | 6 | down | GO REGULATION OF IMMUNE | 0.000 | 0.030 | |
| | | | SYSTEM PROCESS | | | |
| c5 | 6 | down | GO REGULATION OF INNATE | 0.000 | 0.011 | Hspa1a, Hspa1b, Irf1, Irf7, Nfkbia, |
| | | | IMMUNE RESPONSE | | | Nod1, Plscr1, Psmb10, Psmb8, |
| | | | | | | Psmb9, Pum1, Rsad2, Socs1, |
| | | | | | | Socs3, Ubb, Ubc, Ube2k |
| c5 | 6 | down | GO REGULATION OF JNK | 0.000 | 0.002 | Dnaja1, Edn1, Epha4, F2rl1, |
| | | | CASCADE | | | Gadd45b, Gadd45g, Map3k11, |
| | | | | | | Mecom, Nod1, Pdcd4, Plcb1 |
| c5 | 6 | down | GO REGULATION OF JUN | 0.004 | 0.013 | Dnaja1, Edn1, Epha4, Map3k11, |
| | | | KINASE ACTIVITY | | | Mapk8ip3, Pdcd4 |
| c5 | 6 | down | GO REGULATION OF KINASE | 0.000 | 0.000 | Ace, App, Ccdc88a, Ccnl1, Cd81, |
| | | | ACTIVITY | | | Cdkn1a, Cdkn1c, Cyr61, Dnaja1, |
| | | | | | | Dstyk, Dusp1, Dusp26, Edn1, |
| | | | | | | Emp2, Epha4, Gadd45b, Gadd45g, |
| | | | | | | Gas6, Hexim1, Hspa5, Hspb1, Kitl, |
| | | | | | | Klf4, Map3k11, Map4k3, Mapk3, |
| | | | | | | Midn, Nck1, Nod1, Pdcd4, Pdgfb, |
| | | | | | | Pdgfd, Pik3r3, Rapgef2, Rgs3, |
| | | | | | | S1pr2, Sertad1, Socs1, Socs3, |
| | | | | | | Taf7, Tgfb2, Tgfbr2, Ubb, Ubc |
| c5 | 6 | down | GO REGULATION OF LIGASE | 0.017 | 0.045 | |
| | | | ACTIVITY | | | |
| c5 | 6 | down | GO REGULATION OF | 0.006 | 0.043 | |
| | | | LYMPHOCYTE | | | |
| | | | DIFFERENTIATION | | | |
| c5 | 6 | down | GO REGULATION OF MAPK | 0.000 | 0.001 | Arl6ip5, Ash1l, Atf3, Cav2, Cd81, |
| | | | CASCADE | | | Cx3cl1, Cyr61, Dnaja1, Dstyk, |
| | | | | | | Dusp1, Dusp26, Edn1, Epha4, |
| | | | | | | Gadd45b, Gadd45g, Gas6, Id1, |
| | | | | | | Jun, Kitl, Klf4, Map3k11, Map4k3, |
| | | | | | | Mapk3, Mecom, Nod1, Npnt, |
| | | | | | | Nrp1, Pdcd4, Pdgfb, Pdgfd, Plcb1, |
| | | | | | | Ptprr, Rapgef2, Rgs3, S1pr2, |
| | | | | | | Tgfb2, Timp3, Ubb, Ubc |
| c5 | 6 | down | GO REGULATION OF MAP | 0.002 | 0.028 | Cd81, Dnaja1, Dusp1, Edn1, |
| | | | KINASE ACTIVITY | | | Epha4, Gadd45b, Gadd45g, Kitl, |
| | | | | | | Map3k11, Map4k3, Mapk3, Nod1, |
| | | | | | | Pdcd4, Pdgfb, Pdgfd, Rgs3, S1pr2, |
| | | | | | | Ubb, Ubc |
| c5 | 6 | down | GO REGULATION OF MITOTIC | 0.000 | 0.011 | App, Atrx, Btg2, Cav2, Cdk9, |
| | | | CELL CYCLE | | | Cdkn1a, Cdkn1c, Dusp1, Edn1, |
| | | | | | | Hes1, Id2, Maea, Pdgfb, Pim3, |
| | | | | | | Plcb1, Psmb10, Psmb8, Psmb9, |
| | | | | | | Rdx, Sphk1, Ubb, Ubc |
| c5 | 6 | down | GO REGULATION OF MRNA | 0.000 | 0.000 | Btg2, Cdk9, Ddx5, Dhx34, |
| | | | METABOLIC PROCESS | | | Hspa1a, Hspa8, Mbnl2, Sap18, |
| | | | | | | Srsf2, Srsf7, Tob1, Tra2b, Zfp36, |
| | | | | | | Zfp36l2 |
| c5 | 6 | down | GO REGULATION OF MRNA | 0.000 | 0.012 | Ddx5, Hspa8, Mbnl2, Rbfox1, |
| | | | SPLICING VIA SPLICEOSOME | | | Sap18, Srsf2, Srsf7, Tra2b |
| c5 | 6 | down | GO REGULATION OF MUSCLE | 0.015 | 0.037 | |
| | | | CELL DIFFERENTIATION | | | |
| c5 | 6 | down | GO REGULATION OF | 0.000 | 0.005 | Atp6ap1, Fos, Foxp1, Hoxa5, Id2, |
| | | | MYELOID CELL | | | Irf7, Isg15, Jag1, Jun, Kitl, Klf13, |
| | | | DIFFERENTIATION | | | Nfkbia, Prdm16, Rarg, Tob2 |
| c5 | 6 | down | GO REGULATION OF | 0.010 | 0.035 | |
| | | | MYELOID LEUKOCYTE | | | |
| | | | DIFFERENTIATION | | | |
| c5 | 6 | down | GO REGULATION OF NEURAL | 0.008 | 0.023 | Adcyap1, Btg2, Cx3cl1, Dll4, Id2, |
| | | | PRECURSOR CELL | | | Lims2, Vegfc |
| | | | PROLIFERATION | | | |
| c5 | 6 | down | GO REGULATION OF NOTCH | 0.004 | 0.033 | |
| | | | SIGNALING PATHWAY | | | |
| c5 | 6 | down | GO REGULATION OF ORGAN | 0.002 | 0.034 | |
| | | | MORPHOGENESIS | | | |
| c5 | 6 | down | GO REGULATION OF | 0.010 | 0.050 | |
| | | | PEPTIDYL TYROSINE | | | |
| | | | PHOSPHORYLATION | | | |
| c5 | 6 | down | GO REGULATION OF | 0.004 | 0.042 | |
| | | | PHOSPHATIDYLINOSITOL 3 | | | |
| | | | KINASE SIGNALING | | | |
| c5 | 6 | down | GO REGULATION OF | 0.011 | 0.050 | |
| | | | PHOSPHOLIPID METABOLIC | | | |
| | | | PROCESS | | | |
| c5 | 6 | down | GO REGULATION OF PROTEIN | 0.000 | 0.007 | Cdk9, Dnaja1, Dusp1, Egr1, Hfe, |
| | | | MODIFICATION BY SMALL | | | Hspa1a, Hspa1b, Hspa5, Isg15, |
| | | | PROTEIN CONJUGATION OR | | | Psmb10, Psmb8, Psmb9, Sphk1, |
| | | | REMOVAL | | | Topors, Ubb, Ubc |
| c5 | 6 | down | GO REGULATION OF PROTEIN | 0.000 | 0.010 | Ccnl1, Cd81, Cdkn1a, Cdkn1c, |
| | | | SERINE THREONINE KINASE | | | Dnaja1, Dusp1, Edn1, Epha4, |
| | | | ACTIVITY | | | Gadd45b, Gadd45g, Hexim1, |
| | | | | | | Hspb1, Kitl, Map3k11, Map4k3, |
| | | | | | | Mapk3, Nod1, Pdcd4, Pdgfb, |
| | | | | | | Pdgfd, Rapgef2, Rgs3, S1pr2, |
| | | | | | | Sertad1, Ubb, Ubc |
| c5 | 6 | down | GO REGULATION OF | 0.000 | 0.001 | Edn1, Gas6, Hspa1a, Hspa1b, Irf7, |
| | | | RESPONSE TO CYTOKINE | | | Klf4, Socs1, Socs3, Sphk1, Ubb, |
| | | | STIMULUS | | | Ubc |
| c5 | 6 | down | GO REGULATION OF RNA | 0.000 | 0.011 | Ddx5, Esrp2, Hspa1a, Hspa8, |
| | | | SPLICING | | | Mbnl2, Sap18, Son, Srsf2, Srsf7, |
| | | | | | | Tra2b |
| c5 | 6 | down | GO REGULATION OF RNA | 0.002 | 0.005 | Anp32a, Hspa1a, Hspa8, Hspb1, |
| | | | STABILITY | | | Pcid2, Psmb10, Psmb8, Psmb9, |
| | | | | | | Pum1, Ubb, Ubc, Zfp36, Zfp36l1, |
| | | | | | | Zfp36l2 |
| c5 | 6 | down | GO REGULATION OF | 0.000 | 0.000 | Arid5b, Clock, Ddit3, Fos, Gas6, |
| | | | SEQUENCE SPECIFIC DNA | | | Hspa1a, Hspa1b, Id1, Id2, Id3, Jun, |
| | | | BINDING TRANSCRIPTION | | | Klf4, Mapk3, Nfkbia, Nod1, Plpp3, |
| | | | FACTOR ACTIVITY | | | Setd6, Sgk1, Sphk1, Thra, Ubb, |
| | | | | | | Ubc |
| c5 | 6 | down | GO REGULATION OF STEM | 0.000 | 0.002 | H1foo, Hes1, Hoxb4, Jag1, Snai1, |
| | | | CELL DIFFERENTIATION | | | Tgfb2, Tgfbr2, Zfp36l2 |
| c5 | 6 | down | GO REGULATION OF STEM | 0.008 | 0.038 | |
| | | | CELL PROLIFERATION | | | |
| c5 | 6 | down | GO REGULATION OF STRESS | 0.000 | 0.001 | Arl6ip5, Dnaja1, Edn1, Epha4, |
| | | | ACTIVATED PROTEIN KINASE | | | F2rl1, Gadd45b, Gadd45g, |
| | | | SIGNALING CASCADE | | | Map3k11, Mapk3, Mecom, Nod1, |
| | | | | | | Pdcd4, Plcb1, Tgfb2 |
| c5 | 6 | down | GO REGULATION OF | 0.000 | 0.042 | |
| | | | TRANSCRIPTION | | | |
| | | | REGULATORY REGION DNA | | | |
| | | | BINDING | | | |
| c5 | 6 | down | GO REGULATION OF | 0.000 | 0.001 | Ace, App, Ccdc88a, Ccnl1, Cd81, |
| | | | TRANSFERASE ACTIVITY | | | Cdkn1a, Cdkn1c, Cyr61, Dnaja1, |
| | | | | | | Dstyk, Dusp1, Dusp26, Edn1, |
| | | | | | | Emp2, Epha4, Gadd45b, Gadd45g, |
| | | | | | | Gas6, Hexim1, Hspa5, Hspb1, Kitl, |
| | | | | | | Klf4, Map3k11, Map4k3, Mapk3, |
| | | | | | | Midn, Nck1, Nod1, Pdcd4, Pdgfb, |
| | | | | | | Pdgfd, Pik3r3, Psmb10, Psmb8, |
| | | | | | | Psmb9, Rapgef2, Rgs3, S1pr2, |
| | | | | | | Sertad1, Socs1, Socs3, Taf7, |
| | | | | | | Tgfb2, Tgfbr2, Topors, Ubb, Ubc |
| c5 | 6 | down | GO REGULATION OF | 0.000 | 0.001 | Acvrl1, Cav2, Cdkn1c, Cited2, |
| | | | TRANSMEMBRANE RECEPTOR | | | Cyr61, Hes1, Hfe, Hspa5, Ltbp4, |
| | | | PROTEIN SERINE THREONINE | | | Mecom, Msx1, Nov, Npnt, |
| | | | KINASE SIGNALING | | | Prdm16, Skil, Tgfb2, Tgfbr2, |
| | | | PATHWAY | | | Tob1, Ubb, Ubc |
| c5 | 6 | down | GO REGULATION OF TYPE I | 0.004 | 0.030 | |
| | | | INTERFERON PRODUCTION | | | |
| c5 | 6 | down | GO REGULATORY REGION | 0.000 | 0.000 | Arid4a, Arid5b, Atf3, Atf4, Cbx4, |
| | | | NUCLEIC ACID BINDING | | | Cdk9, Clock, Ddit3, Egr1, Fos, |
| | | | | | | Fosb, H3f3b, Hes1, Hoxa5, Irf1, |
| | | | | | | Irf2, Irf7, Irf9, Jun, Junb, Jund, |
| | | | | | | Klf11, Klf13, Klf4, Lmo2, Msx1, |
| | | | | | | Nfe2l1, Nr3c1, Nsd1, Rarg, Rfx1, |
| | | | | | | Skil, Smarca2, Snai1, Sox13, |
| | | | | | | Sox17, Sox18, Taf7, Tbx3, Thra |
| c5 | 6 | down | GO RESPONSE TO ANTIBIOTIC | 0.000 | 0.036 | |
| c5 | 6 | down | GO RESPONSE TO BMP | 0.006 | 0.035 | |
| c5 | 6 | down | GO RESPONSE TO CALCIUM | 0.002 | 0.003 | Anxa5, Dusp1, Edn1, Entpd6, Fos, |
| | | | ION | | | Fosb, Fus, Jun, Junb, Jund |
| c5 | 6 | down | GO RESPONSE TO CYTOKINE | 0.000 | 0.000 | B2m, Bst2, Cdk9, Cx3cl1, Cxcl1, |
| | | | | | | Edn1, Egr1, Fos, Gas6, Hfe, |
| | | | | | | Hspa5, Ifitm3, Irf1, Irf2, Irf7, Irf9, |
| | | | | | | Isg15, Jun, Junb, Jund, Klf2, Lifr, |
| | | | | | | Map4k3, Mapk3, Mcl1, Nfkbia, |
| | | | | | | Npnt, Osmr, Plcb1, Plscr1, |
| | | | | | | Psmb10, Psmb8, Psmb9, Rsad2, |
| | | | | | | Sbno2, Skil, Socs1, Socs3, Sphk1, |
| | | | | | | Tnfsf12, Ubb, Ubc, Vcam1, Xaf1 |
| c5 | 6 | down | GO RESPONSE TO | 0.000 | 0.000 | Atf3, Atf4, Bbc3, Dctn1, Ddit3, |
| | | | ENDOPLASMIC RETICULUM | | | Dnajb9, Hspa5, Jun, Nck1, Pdia4, |
| | | | STRESS | | | Pdia6, Ppp1r15a, Tmx4, Ubxn4 |
| c5 | 6 | down | GO RESPONSE TO | 0.000 | 0.022 | Adamts1, Alpl, Atf3, Atf4, |
| | | | EXTRACELLULAR STIMULUS | | | Cdkn1a, Comt, Ddit3, Fos, |
| | | | | | | Gabarapl2, Gas6, Glul, Hfe, |
| | | | | | | Hspa5, Hspa8, Jun, Ppp1r15a, |
| | | | | | | Rnf152, Socs3, Sphk1, Tgfbr2, |
| | | | | | | Thra, Timp3, Vcam1, Zfp36 |
| c5 | 6 | down | GO RESPONSE TO | 0.000 | 0.001 | Dll4, Dstyk, Ier2, Mapk3, Nr4a1, |
| | | | FIBROBLAST GROWTH | | | Polr2l, Shoc2, Ubb, Ubc |
| | | | FACTOR | | | |
| c5 | 6 | down | GO RESPONSE TO GROWTH | 0.000 | 0.007 | Acvrl1, App, Dll4, Dstyk, Edn1, |
| | | | FACTOR | | | Egr1, Fermt2, Fos, Gas6, Hfe, |
| | | | | | | Hspb1, Id1, Ier2, Jun, Klf4, |
| | | | | | | Mapk3, Msx1, Nr4a1, Nrp1, |
| | | | | | | Pdgfb, Pdgfd, Polr2l, Rapgef2, |
| | | | | | | Skil, Sphk1, Stmn2, Tgfb2, Tgfbr2, |
| | | | | | | Ubb, Ubc, Vcam1 |
| c5 | 6 | down | GO RESPONSE TO HEAT | 0.000 | 0.006 | Cdkn1a, Dnaja1, Hspa1a, Hspa1b, |
| | | | | | | Hspb7, Socs3 |
| c5 | 6 | down | GO RESPONSE TO INORGANIC | 0.000 | 0.012 | Anxa5, App, Atf4, Atrx, B2m, |
| | | | SUBSTANCE | | | Crip1, Ddit3, Dusp1, Edn1, Fos, |
| | | | | | | Fosb, Fus, Hfe, Hspa5, Id2, Jun, |
| | | | | | | Junb, Jund, Kdm6b, Klf2, Klf4, |
| | | | | | | Klf6, Pdgfd, Ppp1r15a, Rhob, |
| | | | | | | S100a13, Sphk1, Vcam1 |
| c5 | 6 | down | GO RESPONSE TO | 0.000 | 0.000 | B2m, Bst2, Cx3cl1, Edn1, Gbp5, |
| | | | INTERFERON GAMMA | | | Hfe, Ifitm3, Irf1, Irf2, Irf7, Irf9, |
| | | | | | | Trim5, Vcam1 |
| c5 | 6 | down | GO RESPONSE TO | 0.000 | 0.006 | Cx3cl1, Edn1, Egr1, Klf2, Mapk3, |
| | | | INTERLEUKIN 1 | | | Plcb1, Psmb9, Sphk1 |
| c5 | 6 | down | GO RESPONSE TO KETONE | 0.002 | 0.004 | Adamts1, Cdkn1a, Dusp1, Edn1, |
| | | | | | | Fos, Fosb, Gas6, Junb, Klf2, Klf4, |
| | | | | | | Klf6, Socs1, Socs3, Sphk1, Tgfb2 |
| c5 | 6 | down | GO RESPONSE TO METAL ION | 0.006 | 0.047 | |
| c5 | 6 | down | GO RESPONSE TO ORGANIC | 0.000 | 0.035 | |
| | | | CYCLIC COMPOUND | | | |
| c5 | 6 | down | GO RESPONSE TO | 0.000 | 0.034 | |
| | | | PROGESTERONE | | | |
| c5 | 6 | down | GO RESPONSE TO RADIATION | 0.002 | 0.010 | App, Atf4, Cdkn1a, Clock, Crip1, |
| | | | | | | Crtc1, Dusp1, Egr1, Fos, Id2, Jun, |
| | | | | | | Junb, Jund, Map4k3, Ppp1cb, |
| | | | | | | Ppp1r15a, Rhob, Sgk1, Socs3, |
| | | | | | | Vcam1 |
| c5 | 6 | down | GO RESPONSE TO REACTIVE | 0.009 | 0.040 | |
| | | | OXYGEN SPECIES | | | |
| c5 | 6 | down | GO RESPONSE TO | 0.000 | 0.000 | Atf3, Atf4, Cdkn1a, Comt, Ddit3, |
| | | | STARVATION | | | Gabarapl2, Gas6, Glul, Hfe, |
| | | | | | | Hspa5, Hspa8, Jun, Ppp1r15a, |
| | | | | | | Rnf152, Sphk1, Zfp36 |
| c5 | 6 | down | GO RESPONSE TO | 0.000 | 0.011 | Cdkn1a, Dnaja1, Fos, Hspa1a, |
| | | | TEMPERATURE STIMULUS | | | Hspa1b, Hspb7, Nfkbia, Socs3, |
| | | | | | | Thra |
| c5 | 6 | down | GO RESPONSE TO | 0.000 | 0.000 | Atf3, Atf4, Ddit3, Dnaja1, Dnajb1, |
| | | | TOPOLOGICALLY INCORRECT | | | Dnajb9, Hspa5, Hspa8, Hspb1, |
| | | | PROTEIN | | | Pdia6, Ppp1r15a, Tor1b, Ubxn4 |
| c5 | 6 | down | GO RESPONSE TO | 0.004 | 0.033 | |
| | | | TRANSFORMING GROWTH | | | |
| | | | FACTOR BETA | | | |
| c5 | 6 | down | GO RESPONSE TO TYPE I | 0.000 | 0.000 | Bst2, Egr1, Hfe, Ifitm3, Irf1, Irf2, |
| | | | INTERFERON | | | Irf6, Irf7, Irf9, Isg15, Psmb8, |
| | | | | | | Rsad2, Xaf1 |
| c5 | 6 | down | GO RNA POLYMERASE II | 0.000 | 0.000 | Arid5b, Atf3, Atf4, Btg2, Clock, |
| | | | TRANSCRIPTION FACTOR | | | Csrnp1, Ddit3, Ebf1, Egr1, Epas1, |
| | | | ACTIVITY SEQUENCE | | | Fos, Fosb, Foxp1, Hes1, hey 1, |
| | | | SPECIFIC DNA BINDING | | | Hoxa5, Irf1, Irf2, Irf7, Jun, Junb, |
| | | | | | | Jund, Klf11, Klf13, Klf4, Lmo2, |
| | | | | | | Msx1, Nfe2l1, Nr3c1, Nr4a1, |
| | | | | | | Plscr1, Rarg, Rfx1, Skil, Snai1, |
| | | | | | | Sox17, Sox18, Tbx3, Thra, Zbtb20 |
| c5 | 6 | down | GO RNA POLYMERASE II | 0.000 | 0.004 | Atf4, Bbs5, Dusp26, Id4, Jun, |
| | | | TRANSCRIPTION FACTOR | | | Klf4, Lmo2, Tbx3 |
| | | | BINDING | | | |
| c5 | 6 | down | GO SEQUENCE SPECIFIC DNA | 0.000 | 0.000 | Arid5b, Atf3, Atf4, Cdk9, Clock, |
| | | | BINDING | | | Crip1, Csrnp1, Ddit3, Egr1, Epas1, |
| | | | | | | Fos, Fosb, Foxp1, H3f3b, Hes1, |
| | | | | | | hey 1, Hlx, Hoxa5, Hoxb4, Irf1, |
| | | | | | | Irf2, Irf7, Irx3, Jun, Junb, Jund, |
| | | | | | | Kdm6b, Klf13, Klf2, Klf4, Lmo2, |
| | | | | | | Msx1, Nfe2l1, Nr3c1, Nr4a1, |
| | | | | | | Nsd1, Prdm16, Rarg, Rfx1, Skil, |
| | | | | | | Snai1, Sox13, Sox17, Sox18, |
| | | | | | | Tbx3, Thra |
| c5 | 6 | down | GO SKELETAL SYSTEM | 0.002 | 0.041 | |
| | | | DEVELOPMENT | | | |
| c5 | 6 | down | GO SKELETAL SYSTEM | 0.003 | 0.039 | |
| | | | MORPHOGENESIS | | | |
| c5 | 6 | down | GO SOMATIC STEM CELL | 0.000 | 0.016 | Braf, Epas1, Hes1, Klf4, Polr2l, |
| | | | POPULATION MAINTENANCE | | | Prdm16, Zfp36l2 |
| c5 | 6 | down | GO STEM CELL | 0.000 | 0.012 | Ace, Edn1, Efnb1, Hes1, hey 1, |
| | | | DIFFERENTIATION | | | Hoxb4, Jag1, Kitl, Mapk3, Msx1, |
| | | | | | | Nrp1, Pum1, Sema3g, Setd6, |
| | | | | | | Snai1, Sox17, Sox18, Tgfb2 |
| c5 | 6 | down | GO S ADENOSYLMETHIONINE | 0.004 | 0.013 | Ash1l, Comt, Mecom, Nsd1, |
| | | | DEPENDENT | | | Pcmtd1, Prdm16, Setd6 |
| | | | METHYLTRANSFERASE | | | |
| | | | ACTIVITY | | | |
| c5 | 6 | down | GO TAXIS | 0.000 | 0.039 | |
| c5 | 6 | down | GO TISSUE MORPHOGENESIS | 0.000 | 0.025 | Acvrl1, Ctsz, Cyr61, Dlg3, Dll4, |
| | | | | | | Edn1, Efnb2, Epha4, Hes1, hey 1, |
| | | | | | | Hoxa5, Hoxb4, Id4, Irx3, Jag1, |
| | | | | | | Kdm6b, Klf4, Msx1, Npnt, Nrarp, |
| | | | | | | Nrp1, Podxl, Rarg, Rhob, Snai1, |
| | | | | | | Socs3, Sox17, Sox18, Tbx3, Tcf15, |
| | | | | | | Tgfb2, Tgfbr2, Topors, Vegfc |
| c5 | 6 | down | GO TRANSCRIPTIONAL | 0.000 | 0.000 | Atf4, Ddit3, Ebf1, Egr1, Epas1, |
| | | | ACTIVATOR ACTIVITY RNA | | | Fos, Fosb, Hoxa5, Irf1, Jun, Junb, |
| | | | POLYMERASE II CORE | | | Klf13, Klf4, Nr3c1, Nr4a1, Plscr1, |
| | | | PROMOTER PROXIMAL | | | Runx2, Sox18 |
| | | | REGION SEQUENCE SPECIFIC | | | |
| | | | BINDING | | | |
| c5 | 6 | down | GO TRANSCRIPTIONAL | 0.000 | 0.000 | Atf3, Atf4, Csrnp1, Ddit3, Ebf1, |
| | | | ACTIVATOR ACTIVITY RNA | | | Egr1, Epas1, Fos, Fosb, Hoxa5, |
| | | | POLYMERASE II | | | Irf1, Irf2, Jun, Junb, Klf13, Klf4, |
| | | | TRANSCRIPTION | | | Lmo2, Msx1, Nfe2l1, Nr3c1, |
| | | | REGULATORY REGION | | | Nr4a1, Plscr1, Runx2, Sox17, |
| | | | SEQUENCE SPECIFIC BINDING | | | Sox18 |
| c5 | 6 | down | GO TRANSCRIPTIONAL | 0.000 | 0.014 | Cited2, Clock, Jun, Klf4, Lmo2, |
| | | | REPRESSOR ACTIVITY RNA | | | Nfe2l1, Rere, Smarca2 |
| | | | POLYMERASE II ACTIVATING | | | |
| | | | TRANSCRIPTION FACTOR | | | |
| | | | BINDING | | | |
| c5 | 6 | down | GO TRANSCRIPTIONAL | 0.004 | 0.005 | Atf3, Btg2, Hes1, Mlxipl, Skil, |
| | | | REPRESSOR ACTIVITY RNA | | | Snai1, Tbx3, Zbtb20 |
| | | | POLYMERASE II CORE | | | |
| | | | PROMOTER PROXIMAL | | | |
| | | | REGION SEQUENCE SPECIFIC | | | |
| | | | BINDING | | | |
| c5 | 6 | down | GO TRANSCRIPTIONAL | 0.000 | 0.003 | Cited2, Clock, Jun, Klf4, Lmo2, |
| | | | REPRESSOR ACTIVITY RNA | | | Nfe2l1, Rere, Smarca2 |
| | | | POLYMERASE II | | | |
| | | | TRANSCRIPTION FACTOR | | | |
| | | | BINDING | | | |
| c5 | 6 | down | GO TRANSCRIPTIONAL | 0.000 | 0.002 | Arid5b, Atf3, Btg2, Hes1, Msx1, |
| | | | REPRESSOR ACTIVITY RNA | | | Skil, Snai1, Tbx3, Zbtb20, Zeb1 |
| | | | POLYMERASE II | | | |
| | | | TRANSCRIPTION | | | |
| | | | REGULATORY REGION | | | |
| | | | SEQUENCE SPECIFIC BINDING | | | |
| c5 | 6 | down | GO TRANSCRIPTIONAL | 0.011 | 0.047 | |
| | | | REPRESSOR COMPLEX | | | |
| c5 | 6 | down | GO TRANSCRIPTION | 0.000 | 0.013 | Arid5b, Arl2bp, Cited2, Ctbp2, |
| | | | COACTIVATOR ACTIVITY | | | Ddx5, Fus, Jun, Junb, Jund, Klf7, |
| | | | | | | Med20, Prdm16, Sfr1, Smarca2, |
| | | | | | | Sox17, Taf7 |
| c5 | 6 | down | GO TRANSCRIPTION | 0.000 | 0.002 | Atf3, Cbx4, Cited2, Ctbp2, Ddit3, |
| | | | COREPRESSOR ACTIVITY | | | Id3, Id4, Junb, Nsd1, Pias4, Sap18, |
| | | | | | | Skil, Skor1, Srsf2, Tdp2, Tob1 |
| c5 | 6 | down | GO TRANSCRIPTION FACTOR | 0.000 | 0.000 | Arid5b, Arl2bp, Atf3, Atf4, Btg1, |
| | | | ACTIVITY PROTEIN BINDING | | | Cbx4, Cited2, Clock, Ctbp2, Ddit3, |
| | | | | | | Ddx5, Fus, hey 1, Id3, Id4, Jun, |
| | | | | | | Junb, Jund, Klf4, Klf7, Lmo2, |
| | | | | | | Med11, Nfe2l1, Nfkbiz, Nr3c1, |
| | | | | | | Nsd1, Prdm16, Sap18, Sfr1, Skil, |
| | | | | | | Smarca2, Sox17, Srsf2, Taf7, |
| | | | | | | Tdp2, Tob1 |
| c5 | 6 | down | GO TRANSCRIPTION FACTOR | 0.000 | 0.000 | Atf3, Atf4, Btg2, Clock, Ddit3, |
| | | | ACTIVITY RNA POLYMERASE | | | Ebf1, Egr1, Epas1, Fos, Fosb, |
| | | | II CORE PROMOTER | | | Hes1, Hoxa5, Irf1, Irf7, Jun, Junb, |
| | | | PROXIMAL REGION | | | Klf13, Klf4, Msx1, Nr3c1, Nr4a1, |
| | | | SEQUENCE SPECIFIC BINDING | | | Plscrl, Runx2, Skil, Snai1, Sox18, |
| | | | | | | Tbx3, Zbtb20 |
| c5 | 6 | down | GO TRANSCRIPTION FACTOR | 0.002 | 0.012 | Atf4, Cited2, Clock, Jun, Klf4, |
| | | | ACTIVITY RNA POLYMERASE | | | Lmo2, Med11, Med20, Nfe2l1, |
| | | | II TRANSCRIPTION FACTOR | | | Nr3c1, Smarca2 |
| | | | BINDING | | | |
| c5 | 6 | down | GO TRANSCRIPTION FACTOR | 0.000 | 0.000 | Atf4, Cdk9, Ctbp2, Ddit3, Dusp26, |
| | | | BINDING | | | Epas1, Fos, Fosb, Fus, Hes1, hey 1, |
| | | | | | | Id1, Id3, Id4, Jun, Junb, Jund, Klf4, |
| | | | | | | Lmo2, Nfkbia, Nsd1, Prdm16, |
| | | | | | | Rarg, Setd6, Sox17, Taf7, Tbx3, |
| | | | | | | Thra, Tob2 |
| c5 | 6 | down | GO TRANSCRIPTION FACTOR | 0.003 | 0.026 | Atf4, Clock, Ddit3, Epas1, Fos, |
| | | | COMPLEX | | | Jun, Junb, Jund, Klf4, Lmo2, Rarg, |
| | | | | | | Sox17, Sox18, Taf1, Taf7 |
| c5 | 6 | down | GO TRANSCRIPTION FROM | 0.000 | 0.000 | Arid4a, Ash1l, Atf3, Atf4, Cdk9, |
| | | | RNA POLYMERASE II | | | Clock, Csrnp1, Ddit3, Ebf1, Egr1, |
| | | | PROMOTER | | | Epas1, Fos, Fosb, Hoxa5, Irf1, Irf2, |
| | | | | | | Irf7, Irf9, Jun, Junb, Jund, Klf11, |
| | | | | | | Klf13, Klf4, Lmo2, Med20, Msx1, |
| | | | | | | Nfe2l1, Nr3c1, Nr4a1, Plscr1, |
| | | | | | | Polr2l, Rarg, Sox17, Sox18, Srsf2, |
| | | | | | | Srsf7, Taf7, Thra |
| c5 | 6 | down | GO TRANSCRIPTION | 0.002 | 0.034 | |
| | | | INITIATION FROM RNA | | | |
| | | | POLYMERASE II PROMOTER | | | |
| c5 | 6 | down | GO TRANSFORMING GROWTH | 0.006 | 0.027 | Acvrl1, Eid2, Fermt2, Fos, Id1, |
| | | | FACTOR BETA RECEPTOR | | | Jun, Pdgfb, Skil, Tgfb2, Tgfbr2, |
| | | | SIGNALING PATHWAY | | | Ubb, Ubc |
| c5 | 6 | down | GO TRANSMEMBRANE | 0.000 | 0.006 | Acvrl1, Cited2, Egr1, Fermt2, Fos, |
| | | | RECEPTOR PROTEIN SERINE | | | Hfe, Id1, Jun, Ltbp4, Mapk3, |
| | | | THREONINE KINASE | | | Msx1, Pdgfb, Runx2, Skil, Tgfb2, |
| | | | SIGNALING PATHWAY | | | Tgfbr2, Tob1, Ubb, Ubc |
| c5 | 6 | down | GO TUBE DEVELOPMENT | 0.000 | 0.034 | |
| c5 | 6 | down | GO TUBE MORPHOGENESIS | 0.003 | 0.039 | |
| c5 | 6 | down | GO T CELL DIFFERENTIATION | 0.000 | 0.016 | B2m, Dll4, Egr1, Foxp1, Irf1, |
| | | | | | | Rora, Rsad2, Zfp36l1, Zfp36l2 |
| c5 | 6 | down | GO T CELL RECEPTOR | 0.012 | 0.050 | |
| | | | SIGNALING PATHWAY | | | |
| c5 | 6 | down | GO UNFOLDED PROTEIN | 0.002 | 0.014 | Dnaja1, Dnajb1, Dnajb6, Hspa1a, |
| | | | BINDING | | | Hspa1b, Hspa5, Hspa8, Ptges3, |
| | | | | | | Tapbp |
| c5 | 6 | down | GO UROGENITAL SYSTEM | 0.003 | 0.017 | Ace, Adamts1, Arid5b, Cdkn1c, |
| | | | DEVELOPMENT | | | Crip1, Efnb2, Egr1, Epha4, Hes1, |
| | | | | | | Id2, Id3, Id4, Irx3, Jag1, Npnt, |
| | | | | | | Nrp1, Pcsk5, Pdgfb, Pecam1, |
| | | | | | | Podxl, Rarg, Sox17, Thra |
| c5 | 6 | down | GO VASCULOGENESIS | 0.014 | 0.049 | |
| c5 | 6 | down | GO VENTRICULAR SEPTUM | 0.000 | 0.004 | Cited2, Cyr61, Fzd2, Hes1, hey 1, |
| | | | DEVELOPMENT | | | Id2, Tbx3 |
| c5 | 6 | up | GO BASEMENT MEMBRANE | 0.000 | 0.049 | Col4a1, Nid1, Nid2, Ramp2 |
| | | | ORGANIZATION | | | |
| c5 | 6 | up | GO CELLULAR RESPONSE TO | 0.000 | 0.028 | Aqp1 |
| | | | NITRIC OXIDE | | | |
| c5 | 6 | up | GO CELLULAR RESPONSE TO | 0.000 | 0.023 | Aqp1 |
| | | | OSMOTIC STRESS | | | |
| c5 | 6 | up | GO COLLAGEN TRIMER | 0.000 | 0.046 | C1ql3, C1qtnf6, Col15a1, Col27a1, |
| | | | | | | Col4a1, Col4a2, Col5a1, Col5a3, |
| | | | | | | Emilin1 |
| c5 | 6 | up | GO COMPLEX OF COLLAGEN | 0.000 | 0.027 | Col27a1, Col4a1, Col4a2, Col5a1, |
| | | | TRIMERS | | | Col5a3 |
| c5 | 6 | up | GO EXTRACELLULAR MATRIX | 0.000 | 0.036 | Adam10, Adamts5, Capg, Hspg2, |
| | | | DISASSEMBLY | | | Lamc1, Mmp14, Nid1 |
| c5 | 6 | up | GO FIBRONECTIN BINDING | 0.000 | 0.002 | Igfbp3, Igfbp5, Itgb1, Thbs4 |
| c5 | 6 | up | GO NEGATIVE REGULATION | 0.000 | 0.020 | Igfbp5 |
| | | | OF MUSCLE HYPERTROPHY | | | |
| c5 | 6 | up | GO NEGATIVE REGULATION | 0.000 | 0.016 | Igfbp3, Igfbp5, Sema6d, Serpine1 |
| | | | OF SMOOTH MUSCLE CELL | | | |
| | | | MIGRATION | | | |
| c5 | 6 | up | GO POSITIVE REGULATION OF | 0.000 | 0.019 | Igfbp3, Igfbp5 |
| | | | INSULIN LIKE GROWTH | | | |
| | | | FACTOR RECEPTOR | | | |
| | | | SIGNALING PATHWAY | | | |
| c5 | 6 | up | GO PROTEIN | 0.001 | 0.048 | Anxa2, Insr, S100a10 |
| | | | HETEROTETRAMERIZATION | | | |
| c5 | 6 | up | GO REGULATION OF FEMALE | 0.000 | 0.008 | Insr, Nupr1 |
| | | | GONAD DEVELOPMENT | | | |
| c5 | 6 | up | GO REGULATION OF INSULIN | 0.000 | 0.018 | Igfbp3, Igfbp5 |
| | | | LIKE GROWTH FACTOR | | | |
| | | | RECEPTOR SIGNALING | | | |
| | | | PATHWAY | | | |
| c5 | 6 | up | GO REGULATION OF | 0.003 | 0.019 | Serpine1 |
| | | | MONOCYTE CHEMOTAXIS | | | |
| c5 | 6 | up | GO RESPONSE TO SALT | 0.000 | 0.047 | Aqp1, Hif1a |
| | | | STRESS | | | |
| c5 | 6 | up | GO WATER TRANSPORT | 0.004 | 0.049 | Aqp1 |
| c5 | 7 | up | GO APOPTOTIC CELL | 0.000 | 0.029 | Axl, Itgav, Lrp1, Scarb1, Tgm2 |
| | | | CLEARANCE | | | |
| c5 | 7 | up | GO KIDNEY MESENCHYME | 0.000 | 0.038 | Pdgfrb, Stat1, Wnt4, Wt1 |
| | | | DEVELOPMENT | | | |
| c5 | 7 | up | GO | 0.000 | 0.038 | Timp1, Timp2, Timp3 |
| | | | METALLOENDOPEPTIDASE | | | |
| | | | INHIBITOR ACTIVITY | | | |
| c5 | 7 | up | GO METANEPHRIC | 0.000 | 0.031 | Pdgfrb, Stat1, Wnt4, Wt1 |
| | | | MESENCHYME | | | |
| | | | DEVELOPMENT | | | |
| c5 | 7 | up | GO PLATELET DERIVED | 0.000 | 0.047 | Col6a1, Pdgfra, Pdgfrb |
| | | | GROWTH FACTOR BINDING | | | |
| c5 | 7 | up | GO RETINA VASCULATURE | 0.000 | 0.028 | Cyp1b1, Pdgfra, Pdgfrb |
| | | | DEVELOPMENT IN CAMERA | | | |
| | | | TYPE EYE | | | |
| c5 | 7 | up | GO SCAVENGER RECEPTOR | 0.000 | 0.002 | Ackr3, Cxcl16, Enpp1, Enpp2, |
| | | | ACTIVITY | | | Lgals3bp, Loxl2, Scara3 |
| c5 | 7 | up | GO VIRUS RECEPTOR | 0.000 | 0.042 | Axl, Hspa1a, Itgav, Itgb5, Scarb1 |
| | | | ACTIVITY | | | |
| c5 | 9 | up | GO ATRIAL SEPTUM | 0.006 | 0.027 | Cyr61, Gja5, Sox4 |
| | | | MORPHOGENESIS | | | |
| c5 | 9 | up | GO BONE RESORPTION | 0.000 | 0.046 | Ctsk, Nox4 |
| c5 | 9 | up | GO CHEMOATTRACTANT | 0.000 | 0.003 | Cxcl12, Lgals3, Mif |
| | | | ACTIVITY | | | |
| c5 | 9 | up | GO EMBRYONIC PLACENTA | 0.000 | 0.003 | Adm, Cdkn1c, Cyr61, Socs3 |
| | | | MORPHOGENESIS | | | |
| c6 | 0 | down | GCNP SHH UP EARLY.V1 DN | 0.000 | 0.048 | Cited2, Egr1, Id2, Irf3, Map4k4, |
| | | | | | | Nr5a2, Sox18, Srrm2, Tm4sf1 |
| c6 | 0 | down | HINATA NFKB IMMU INF | 0.004 | 0.011 | Nfkbia, Ptx3, Tnfaip2 |
| c6 | 0 | down | PDGF ERK DN.V1 DN | 0.000 | 0.004 | Btg1, Cebpb, Dnajb1, Dnajb9, |
| | | | | | | Egr1, Hinfp, Jun, Junb, Maff, |
| | | | | | | Nfil3, Nfkbia, Pnrc1, Rhob, |
| | | | | | | Sult1a1, Trib1 |
| c6 | 0 | down | PKCA DN.V1 DN | 0.000 | 0.014 | Apof, Cfd, Chst1, Enpp4, Malat1, |
| | | | | | | Nfil3, Nfkbiz, Sik1, Trib1, Zfp503 |
| c6 | 0 | up | BCAT.100 UP.V1 DN | 0.008 | 0.028 | Igfbp5, Klf10 |
| c6 | 0 | up | WNT UP.V1 DN | 0.000 | 0.049 | Cd93, Csf2rb2, Lbp, Ramp3, |
| | | | | | | Rasd1, Stc1, Vim |
| c6 | 11 | down | BMI1 DN.V1 UP | 0.000 | 0.001 | Csf1, Edn1, Jag1, Ltbp4, Mmp7, |
| | | | | | | Nrp2, Podxl, Sema3g, Vegfc |
| c6 | 11 | down | LTE2 UP.V1 DN | 0.000 | 0.000 | Atf3, Bst2, Btg3, Dlc1, Fto, Irf7, |
| | | | | | | Jag1, Klf4, Notch1, Ppm1e, |
| | | | | | | Psmb9, Tm4sf1 |
| c6 | 12 | down | CYCLIN D1 UP.V1 UP | 0.000 | 0.045 | D430041D05Rik, Id1, Klf4, Msn, |
| | | | | | | Myt1, Slc12a4, Thy1 |
| c6 | 12 | down | HINATA NFKB IMMU INF | 0.011 | 0.039 | Cxcl1, Irf1, Nfkbia, Tnfaip2 |
| c6 | 12 | down | IL15 UP.V1 UP | 0.000 | 0.045 | Cnnm4, Gadd45b, Rgs16, S100a1, |
| | | | | | | Tnfrsf12a, Tnfrsf21 |
| c6 | 12 | down | KRAS.600.LUNG.BREAST | 0.000 | 0.042 | Col5a3, Cryab, Ephx3, Gamt, |
| | | | UP.V1 DN | | | Gas1, Htr1d, Olfml2a |
| c6 | 12 | down | TGFB UP.V1 UP | 0.000 | 0.034 | Atf3, Cryab, Gadd45b, Jmjd6, |
| | | | | | | Kdm6b, Klf4, Ngf, Rrad, Skil, |
| | | | | | | Slc17a3 |
| c6 | 15 | up | CORDENONSI YAP | 0.000 | 0.030 | Agfg2, Axl, Lhfp, Sgk1, Tgm2, |
| | | | CONSERVED SIGNATURE | | | Tns1 |
| c6 | 15 | up | HINATA NFKB MATRIX | 0.008 | 0.028 | Arhgdia, Fn1 |
| c6 | 16 | up | CAHOY NEURONAL | 0.000 | 0.016 | Camk2b, Epha7, Lin7b, Metrn, |
| | | | | | | Nsf, Prss12, Slc25a22 |
| c6 | 1 | down | BCAT.100 UP.V1 UP | 0.000 | 0.002 | Bmp4, Crym, Efemp1, Eno2, |
| | | | | | | Gadd45g, Runx1 |
| c6 | 1 | down | BCAT GDS748 UP | 0.002 | 0.004 | Bambi, Cebpb, Gadd45g, Runx1 |
| c6 | 1 | down | BMI1 DN MEL18 DN.V1 UP | 0.004 | 0.037 | Csf1, Cxcl1, Grem1, March3, Mt1, |
| | | | | | | Pappa, Pim1, Vegfc, Zeb2 |
| c6 | 1 | down | CAMP UP.V1 UP | 0.006 | 0.034 | Azin1, Eln, Hax1, Hspa5, Ifrd1, |
| | | | | | | Mettl17, Mt2, Myc, Ppp1r15a, |
| | | | | | | Sat2, Taf1d |
| c6 | 1 | down | E2F1 UP.V1 DN | 0.004 | 0.039 | Acox1, Adamts1, Angptl4, Ccl2, |
| | | | | | | Csf1, Cxcl1, Ebf1, Ghr, Klf2, Mdk, |
| | | | | | | Myc, Nfkbiz, Notch2, Sema4b, |
| | | | | | | Sgk1 |
| c6 | 1 | down | GCNP SHH UP EARLY.V1 UP | 0.002 | 0.024 | Adamts1, Azin1, Csf1, Dio1, |
| | | | | | | Rbm39, Rgcc |
| c6 | 1 | down | GLI1 UP.V1 UP | 0.038 | 0.037 | Fem1a, Lmo7, Plxna2 |
| c6 | 1 | down | HINATA NFKB IMMU INF | 0.000 | 0.001 | Ccl2, Cxcl1, Cxcl5, Icam1, Irf1, |
| | | | | | | Nfkbia, Ptx3 |
| c6 | 1 | down | IL2 UP.V1 UP | 0.008 | 0.049 | Cxcl12, Klf6, Pim1, Rgcc, Rgs3, |
| | | | | | | Rhob, Slc1a5, Socs1, Trib1, Vsnl1 |
| c6 | 1 | down | KRAS.300 UP.V1 DN | 0.019 | 0.049 | Brdt, C5ar2, Ceacam5, Cldn16, |
| | | | | | | Dpt, Gadd45g, Gkn1, Krt13, |
| | | | | | | Krt16, Krt4, Lfng, Muc5ac, Rrad, |
| | | | | | | Ryr2, Serpinb13, Slc3a1, Sprr3, |
| | | | | | | Tnfsf10, Tnni3, Ybx2 |
| c6 | 1 | down | KRAS.50 UP.V1 DN | 0.006 | 0.028 | C5ar2, Gadd45g, Krt13, Krt16, |
| | | | | | | Krt4, Rrad, Serpinb13, Sprr3 |
| c6 | 1 | down | KRAS.DF.V1 UP | 0.006 | 0.047 | Akt3, Cxcl1, Grem1, Pid1, Rrad, |
| | | | | | | Vegfc |
| c6 | 1 | down | PDGF ERK DN.V1 DN | 0.002 | 0.036 | Cebpb, Dnajb1, Klf10, Myc, |
| | | | | | | Nfkbia, Rhob, Trib1 |
| c6 | 1 | down | PTEN DN.V2 DN | 0.000 | 0.035 | Bambi, Bmp4, Mid1, Mt1, |
| | | | | | | Serpinh1, Sgk1 |
| c6 | 1 | down | RELA DN.V1 UP | 0.011 | 0.046 | Ccl2, Chst1, Clk1, Fosb, Ints6, |
| | | | | | | Klf10, Klf6, Lmo7, Ppp1r15a, Tnc, |
| | | | | | | Zfp36 |
| c6 | 1 | down | SNF5 DN.V1 DN | 0.002 | 0.024 | Angptl4, Dpep1, Efemp1, Malat1, |
| | | | | | | Mylk, Nfkbiz, Nnmt, Npr3, Pappa, |
| | | | | | | Rrad |
| c6 | 1 | down | STK33 NOMO UP | 0.002 | 0.038 | Cxcl1, Dennd1b, Gja1, Icam1, |
| | | | | | | Kitl, Klf6, Pid1, Ppp1r15a, Rhob, |
| | | | | | | Sgk1, St3gal1, Zeb2, Zfp36 |
| c6 | 1 | down | STK33 UP | 0.000 | 0.028 | Cxcl1, Gja1, Gnpda2, Icam1, Kitl, |
| | | | | | | Klf6, Lfng, Pid1, Ppp1r15a, Rhob, |
| | | | | | | Sgk1, Trib1, Zeb2, Zfp36 |
| c6 | 1 | down | TGFB UP.V1 DN | 0.000 | 0.024 | Adamts1, Angptl4, Cxcl1, Id1, |
| | | | | | | Irf1, Kitl, Plxna2, Slc35a1, Slc5a3, |
| | | | | | | Spry1 |
| c6 | 1 | up | CAHOY ASTROGLIAL | 0.000 | 0.000 | Aebp1, Fbln5, Igfbp3, Lox, Prss23, |
| | | | | | | Serpine1, Serping1, Timp1, |
| | | | | | | Tnfrsf12a, Wisp2 |
| c6 | 1 | up | CRX DN.V1 DN | 0.000 | 0.000 | Anxa2, Cdkn1a, Cxcl13, Inhbb, |
| | | | | | | Nupr1, Pcolce, Rnase4, S100a16, |
| | | | | | | Serping1, Tgm2, Timp1, Tnfrsf12a |
| c6 | 1 | up | ESC V6.5 UP EARLY.V1 DN | 0.000 | 0.001 | Aebp1, Anxa2, Anxa3, Axl, |
| | | | | | | Bhlhe40, Cald1, Gbp2, Igfbp3, |
| | | | | | | Inhbb, Lox, Mgp, Nupr1, Pcolce, |
| | | | | | | Scara3, Serpine1, Timp2, |
| | | | | | | Tnfrsf12a |
| c6 | 1 | up | ESC V6.5 UP LATE.V1 DN | 0.002 | 0.044 | Gadd45a, Inhbb, Lox, Mgp, Msc, |
| | | | | | | Nupr1, Pcolce, Serpine1, Timp1 |
| c6 | 1 | up | HINATA NFKB MATRIX | 0.006 | 0.044 | Serpine1 |
| c6 | 1 | up | KRAS.AMP.LUNG UP.V1 DN | 0.000 | 0.022 | Aldh1a2, Cep41, Enpp1, Trf, |
| | | | | | | Wisp2 |
| c6 | 1 | up | LTE2 UP.V1 DN | 0.000 | 0.049 | Anxa3, Gadd45a, Igf1r, Lgals1, |
| | | | | | | Lpgat1, Mgp, Prss23, Tap1, Ugcg, |
| | | | | | | Wisp2 |
| c6 | 1 | up | LTE2 UP.V1 UP | 0.000 | 0.045 | Actg2, Akap12, Atp6v0a4, |
| | | | | | | Atp6v1h, Cd302, Cgrrf1, Cyp1b1, |
| | | | | | | Dab2, Epas1, Epn3, Golm1, Gpx3, |
| | | | | | | Igfbp5, Insig2, Maoa, Mgat4a, |
| | | | | | | Nova1, Nudt4, Pcdh9, Plcb4, |
| | | | | | | Pros1, Scara3, Serpina1a, Stau2 |
| c6 | 1 | up | MEK UP.V1 DN | 0.002 | 0.048 | Cbr4, Cetn3, Fastkd1, Fto, Igf1r, |
| | | | | | | Insig2, Lpgat1, Mgp, Nol8, Tap1, |
| | | | | | | Wisp2 |
| c6 | 1 | up | MEK UP.V1 UP | 0.000 | 0.046 | Adm, Apod, B3gnt3, Camk2n1, |
| | | | | | | Cldn4, Cyp1b1, Epas1, Gpx3, |
| | | | | | | Limch1, Pdk4, Timp2 |
| c6 | 1 | up | RAPA EARLY UP.V1 DN | 0.000 | 0.031 | Axl, Crip2, Hspa1a, Igfbp3, Pink1, |
| | | | | | | Snta1, Tspan4 |
| c6 | 1 | up | SIRNA EIF4GI UP | 0.002 | 0.014 | Nsg1, Txnip, Wisp2 |
| c6 | 1 | up | TBK1.DN.48HRS DN | 0.010 | 0.024 | Hspa1a, Ier3, Psmb8 |
| c6 | 2 | down | CSR EARLY UP.V1 UP | 0.000 | 0.011 | Amd1, Cbfb, Crispld2, Ddit4l, F3, |
| | | | | | | Klf10, Klf13, Lrrc8a, Mnat1, |
| | | | | | | Myo9b, Nrip1, Nt5e, Smad7, |
| | | | | | | Top1, Ube2n, Vgll4, Ywhag |
| c6 | 2 | down | CSR LATE UP.V1 UP | 0.001 | 0.014 | Cbfb, Crispld2, Drap1, Mras, Nmb, |
| | | | | | | Slc25a11, Timp1, Wsb2 |
| c6 | 2 | down | ERB2 UP.V1 DN | 0.000 | 0.002 | 2010111I01Rik, Btg3, Id2, Klf10, |
| | | | | | | Mgp, Npepps, Nrip1, Slc26a2, |
| | | | | | | Slc6a14, Smad7, Tm4sf1, Tmed5 |
| c6 | 2 | down | HOXA9 DN.V1 UP | 0.000 | 0.002 | Acyp2, Atxn1, Crispld2, |
| | | | | | | D5Ertd579e, Dicer1, Fos, Fry, |
| | | | | | | Fzd2, Ier5, Klf4, Map4k4, Rabac1, |
| | | | | | | Scpep1, Slc26a2, St3gal6, Tob1 |
| c6 | 2 | down | LTE2 UP.V1 DN | 0.001 | 0.028 | Atf3, Atp8a2, Bhlhe41, Btg3, |
| | | | | | | Gem, Hes1, Id1, Ifrd1, Jmjd1c, |
| | | | | | | Klf4, Lgr4, Mgp, Nrip1, Stk26, |
| | | | | | | Tm4sf1 |
| c6 | 2 | down | MEK UP.V1 DN | 0.000 | 0.000 | 201011I01Rik, Atf3, Auts2, Id2, |
| | | | | | | Jmjd1c, Marf1, Mgp, Npepps, |
| | | | | | | Nrep, Nrip1, Pcp4, Sec24d, |
| | | | | | | Slc26a2, Smad7, Stxbp3, Tm4sf1 |
| c6 | 2 | down | PRC2 EZH2 UP.V1 DN | 0.000 | 0.000 | Angptl2, Ank, Atxn1, Col13a1, |
| | | | | | | Hs3st3a1, Klf13, Klf4, Npas2, |
| | | | | | | Nr3c2, Nr4a2, Paqr5, Rgcc, Srpx2, |
| | | | | | | Trib1 |
| c6 | 2 | down | TBK1.DF DN | 0.000 | 0.019 | Atp2b1, Cbfb, Dicer1, Dnajc3, |
| | | | | | | Ehf, Ell2, Fos, Ide, Klf13, Ktn1, |
| | | | | | | Letm1, Ncoa3, Ndufa13, Nrip1, |
| | | | | | | Rala, Rbbp5, Rsrc1, Scarb1, |
| | | | | | | Sec63, Sema3c, Slc26a2, Spin1, |
| | | | | | | Stk26, Ubxn4 |
| c6 | 2 | down | TGFB UP.V1 UP | 0.001 | 0.015 | Abtb2, Atf3, Bcor, Bhlhe40, |
| | | | | | | Chst11, Ell2, Gadd45b, Jmjd1c, |
| | | | | | | Kdm6b, Klf10, Klf4, Lmcd1, |
| | | | | | | Rgcc, S100a3, Skil, Slc26a2, |
| | | | | | | Trib1, Ubl3 |
| c6 | 3 | down | CYCLIN D1 KE .V1 UP | 0.000 | 0.030 | Col5a2, Crip1, Fam3c, Htr6, Id1, |
| | | | | | | Kdm5c, Klf4, Lor, Mall, Pfkl, |
| | | | | | | Pkd1, Prph, Reep5, Thbd |
| c6 | 3 | down | EGFR UP.V1 UP | 0.000 | 0.042 | Atf3, Col5a2, Dusp6, Egln3, |
| | | | | | | Hbegf, Klf4, Ktn1, Mall, Maoa, |
| | | | | | | Mgll, Tnfrsf11b |
| c6 | 3 | down | HINATA NFKB IMMU INF | 0.049 | 0.034 | Nfkbia, Rela |
| c6 | 3 | down | IL15 UP.V1 UP | 0.000 | 0.004 | Adam19, Bmp2, Dusp6, Fam3c, |
| | | | | | | Gadd45b, Hs3st1, Maff, Mpp6, |
| | | | | | | Pim1, Plec, Rgcc, Rhob, S100a1, |
| | | | | | | Sh2d4a, Tnfrsf12a |
| c6 | 3 | down | IL2 UP.V1 UP | 0.000 | 0.010 | Bmp2, Dusp6, Ehd2, Flt1, |
| | | | | | | Gadd45b, Maff, Mpp6, Pim1, Plec, |
| | | | | | | Rgcc, Rhob, Sh2d4a, Tnfrsf12a |
| c6 | 3 | down | PDGF ERK DN.V1 UP | 0.000 | 0.029 | Colla1, Hcrtr1, Hdlbp, Hspa4l, |
| | | | | | | Maoa, Nmbr, Pdia3, Pmp22, Raly1, |
| | | | | | | Rrbp1 |
| c6 | 3 | up | ALK DN.V1 UP | 0.002 | 0.046 | Chil1, Ddit4, Enpp2, Fap, Mt1, |
| | | | | | | Olfml3, Scn1a, Spp1 |
| c6 | 3 | up | RB P130 DN.V1 DN | 0.000 | 0.035 | B2m, Bnip3, Ctsb, Ctsk, Hacd2, |
| | | | | | | Lamp2, Neat1 |
| c6 | 3 | up | SIRNA EIF4GI UP | 0.000 | 0.027 | Btg2, Nsg1, Psap, Txnip, Wisp2, |
| | | | | | | Zfp36l2 |
| c6 | 5 | down | PDGF ERK DN.V1 UP | 0.000 | 0.036 | Anxa11, Atf6, Col1a1, Gtf2h5, |
| | | | | | | Pmp22, Rrbp1 |
| c6 | 5 | up | ALK DN.V1 UP | 0.000 | 0.020 | Angptl4, Ddit4, Enpp2, Mt1, |
| | | | | | | Thap3 |
| c6 | 5 | up | BCAT.100 UP.V1 UP | 0.005 | 0.044 | Enpp2, Gadd45g, Lbp |
| c6 | 5 | up | BCAT GDS748 DN | 0.000 | 0.015 | Ddit4, Fam46a |
| c6 | 5 | up | HINATA NFKB MATRIX | 0.006 | 0.030 | Mmp14, Serpine1 |
| c6 | 5 | up | IL15 UP.V1 DN | 0.000 | 0.002 | Ccng2, Cdkn1c, Deptor, Fgfr1, |
| | | | | | | Hbp1, Pik3ip1, Tns3, Tsc22d3, |
| | | | | | | Txnip |
| c6 | 5 | up | IL2 UP.V1 DN | 0.000 | 0.039 | Cacna1e, Ccng2, Cdkn1c, Deptor, |
| | | | | | | Hbp1, Lipf, Pbx1, Tmem140, |
| | | | | | | Tns3, Trpm1, Txnip |
| c6 | 5 | up | RB P130 DN.V1 DN | 0.000 | 0.050 | B2m, Ctsb, Gadd45a, Hacd2, |
| | | | | | | Lamp2, Tmem140 |
| c6 | 5 | up | SIRNA EIF4GI UP | 0.000 | 0.004 | Btg2, Nsg1, Psap, Txnip, Wisp2 |
| c6 | 6 | down | ATF2 S UP.V1 DN | 0.004 | 0.011 | Crip2, Fbln2, Fos, Id3, Id4, Igfbp2, |
| | | | | | | Magix, Mn1, Neat1, Osmr, Sbno2, |
| | | | | | | St6galnac2, Stmn2, Vcam1 |
| c6 | 6 | down | BMI1 DN.V1 DN | 0.000 | 0.008 | Bst2, Efhd1, Egr1, Epha4, Fos, |
| | | | | | | Id4, Pdcd4, Pik3r3, Snai1, Suclg2 |
| c6 | 6 | down | BMI1 DN MEL18 DN.V1 DN | 0.000 | 0.009 | Bst2, Cfap20, Efhd1, Epha4, Fos, |
| | | | | | | Id2, Id4, Irf9, Kcnv1, Neat1, Pdcd4 |
| c6 | 6 | down | BMI1 DN MEL18 DN.V1 UP | 0.006 | 0.025 | Adgre5, Atf3, Cxcl1, Edn1, Jag1, |
| | | | | | | Klf7, Krt75, Mgll, Podxl, Tgfb2, |
| | | | | | | Timp3, Vegfc |
| c6 | 6 | down | CAMP UP.V1 DN | 0.007 | 0.047 | Arhgap5, Dnaja1, Dynll1, Fosb, |
| | | | | | | Hspa1a, Hspa8, Id2, Pls3, Srrm1, |
| | | | | | | Srsf2, Syt11, Zfp36 |
| c6 | 6 | down | DCA UP.V1 DN | 0.006 | 0.045 | Cx3cl1, Nr4a1, Rnpepl1, Skil, |
| | | | | | | Slc6a6, Sncb |
| c6 | 6 | down | EGFR UP.V1 DN | 0.002 | 0.019 | Bst2, Calcr, Efhd1, Efnb2, Herc6, |
| | | | | | | Id1, Id2, Mettl3, Pdcd4, Sh3glb2, |
| | | | | | | Slc27a3, Ssfa2 |
| c6 | 6 | down | ESC J1 UP LATE.V1 DN | 0.005 | 0.043 | Edn1, Egr1, Klf4, Med11, Socs3, |
| | | | | | | Tcf15, Vegfc |
| c6 | 6 | down | GCNP SHH UP EARLY.V1 DN | 0.000 | 0.005 | Calcr, Cbx1, Cited2, Egr1, Etl4, |
| | | | | | | Gbp7, Hes1, Htr1a, Id2, Ier2, |
| | | | | | | Sox18, Srrm2, Tm4sf1 |
| c6 | 6 | down | LEF1 UP.V1 DN | 0.004 | 0.027 | Clec2d, Cxcl1, Efhd1, Efnb2, |
| | | | | | | Gde1, Herc6, Id1, Id3, Tm4sf1 |
| c6 | 6 | down | LTE2 UP.V1 DN | 0.000 | 0.007 | Atf3, Bst2, Herc6, Hes1, Id1, Ifrd1, |
| | | | | | | Irf7, Irf9, Isg15, Jag1, Klf4, |
| | | | | | | Lgals3bp, Nrp1, Plcb1, Plscr1, |
| | | | | | | Psmb9, Tm4sf1, Usp25 |
| c6 | 6 | down | MEL18 DN.V1 DN | 0.000 | 0.000 | Bst2, Cfap20, Crip1, Efhd1, Egr1, |
| | | | | | | Fos, Id2, Id4, Irf9, Plcb1, Sulf1 |
| c6 | 6 | down | MTOR UP.N4.V1 DN | 0.000 | 0.007 | Anxa5, Arid5b, Btg2, Efhc1, Eogt, |
| | | | | | | Fbln5, Hes1, Neat1, Pdcd4, Pnrc1, |
| | | | | | | Syne2, Zbtb20 |
| c6 | 6 | down | PDGF ERK DN.V1 DN | 0.000 | 0.000 | Arf6, Btg1, Dhx34, Dnajb1, |
| | | | | | | Dnajb9, Dusp1, Efnb2, Egr1, Irf2, |
| | | | | | | Jun, Junb, Mapk3, Nfkbia, Pnrc1, |
| | | | | | | Ppp1r10, Rhob, Tmed5, Zfp36l2 |
| c6 | 6 | down | PDGF UP.V1 DN | 0.002 | 0.006 | Acvrl1, Dnmbp, Etl4, Jun, Mapk3, |
| | | | | | | Msx1, Timp3 |
| c6 | 6 | down | PDGF UP.V1 UP | 0.002 | 0.010 | Atf3, Cbx4, Cdkn1a, Chka, |
| | | | | | | Cldnd1, Egr1, Fos, Glod4, Las1l, |
| | | | | | | Nedd9, Parg, Slc20a1, Uap1 |
| c6 | 6 | down | PTEN DN.V2 DN | 0.006 | 0.026 | Apold1, Copz2, Crip2, Kcne2, |
| | | | | | | Loxl1, Mapk3, Sgk1, Sphk1, |
| | | | | | | St6galnac2, Tbx3 |
| c6 | 6 | down | RB DN.V1 UP | 0.000 | 0.006 | Bbc3, Btg2, Crip2, Ephx1, Hes1, |
| | | | | | | Id1, Ier2, Palmd, Sulf2, Thra |
| c6 | 6 | down | RB P107 DN.V1 UP | 0.000 | 0.011 | Cd81, Cdkn1c, Dusp1, Fbln2, |
| | | | | | | Fbln5, Lgals3bp, Loxl1, Prdx4, |
| | | | | | | Sncg |
| c6 | 6 | down | RELA DN.V1 UP | 0.004 | 0.029 | Atf3, Egr1, Fos, Fosb, Gadd45b, |
| | | | | | | Htr1f, Jun, Junb, Klf6, Ppp1r15a, |
| | | | | | | Snai1, Zfp36, Zfp36l1 |
| c6 | 6 | down | SIRNA EIF4GI UP | 0.013 | 0.043 | Atf3, Btg2, Eif3k, Hspb1, Zfp36l2 |
| c6 | 6 | down | STK33 NOMO UP | 0.000 | 0.000 | Arid5b, Btg2, Csrnp1, Cxcl1, |
| | | | | | | Dolk, Dusp1, Efnb2, Epas1, Fos, |
| | | | | | | Ier3, Jun, Junb, Kitl, Klf6, |
| | | | | | | Map4k3, Midn, Neat1, Nrp1, |
| | | | | | | Nuak1, Ppp1r15a, Rhob, Sar1b, |
| | | | | | | Sgk1, Sipa1l2, Tm6sf1, Zfp36, |
| | | | | | | Zfp36l1 |
| c6 | 6 | down | STK33 SKM UP | 0.005 | 0.012 | Arid5b, Atrx, Btg2, Cdkn1a, |
| | | | | | | Cdkn1c, Dusp1, Egr1, Fnbp1l, Fos, |
| | | | | | | Irf1, Irf9, Junb, Kitl, Klf6, Mfap3, |
| | | | | | | Midn, Nfkbia, Nfkbiz, Nov, Rhob, |
| | | | | | | Sgk1, Sipa1l2, Skil, Zfp36 |
| c6 | 6 | down | STK33 UP | 0.000 | 0.007 | Arid5b, Btg2, Cdkn1a, Csrnp1, |
| | | | | | | Cxcl1, Dusp1, Egr1, Fnbp1l, Fos, |
| | | | | | | Irf9, Jun, Junb, Kitl, Klf6, Map4k3, |
| | | | | | | Mfap3, Midn, Nov, Nrp1, Nuak1, |
| | | | | | | Ppp1r15a, Rhob, Sgk1, Sipa1l2, |
| | | | | | | Zfp36, Zfp36l1 |
| c6 | 6 | down | TGFB UP.V1 UP | 0.002 | 0.011 | Atf3, Edn1, Epha4, Fermt2, |
| | | | | | | Fnbp1l, Gadd45b, Hlx, Id3, Jun, |
| | | | | | | Junb, Kdm6b, Klf4, Klf7, Pdgfb, |
| | | | | | | Rapgef2, Rhob, Sgk1, Skil, Snai1, |
| | | | | | | Sphk1, Tbx3, Tulp4, Zfp36l1 |
| c6 | 6 | up | BCAT.100 UP.V1 DN | 0.000 | 0.004 | 8430408G22Rik, Igfbp5 |
| c6 | 6 | up | BRCA1 DN.V1 UP | 0.000 | 0.001 | Cd34, Klk8, Ltc4s, Timp4 |
| c6 | 6 | up | HINATA NFKB MATRIX | 0.000 | 0.008 | Mmp14, Serpine1 |
| c6 | 7 | up | AKT UP.V1 DN | 0.000 | 0.008 | Ackr3, Angptl4, Fam20c, Gas1, |
| | | | | | | Htra1, Id3, Il13ra1, Paqr7, Pdgfra, |
| | | | | | | Plpp3, Scara3, Timp3, Tspan4 |
| c6 | 7 | up | AKT UP.V1 UP | 0.001 | 0.017 | Cxcl16, Insig2, Klf6, Pon2, |
| | | | | | | Tnfrsf12a |
| c6 | 7 | up | ALK DN.V1 UP | 0.013 | 0.041 | Angptl4, Ccl20, Cxcl5, Ddit4, |
| | | | | | | Dmp1, Enpp2, Pdzd7, Spp1, |
| | | | | | | Steap4, Thap3 |
| c6 | 7 | up | CAHOY ASTROGLIAL | 0.000 | 0.005 | Aebp1, Lbp, Lox, Loxl2, Ltbp2, |
| | | | | | | Prss23, Serpine1, Serping1, Timp1, |
| | | | | | | Tnfrsf12a, Wisp2 |
| c6 | 7 | up | CORDENONSI YAP | 0.001 | 0.011 | Ankrd1, Axl, Ctgf, Dusp1, Emp2, |
| | | | CONSERVED SIGNATURE | | | Flna, Itgb5, Serpine1, Tgm2 |
| c6 | 7 | up | CRX DN.V1 DN | 0.000 | 0.001 | Cdkn1a, Fkbp5, Inhbb, Junb, |
| | | | | | | Nupr1, Plec, S100a6, Serping1, |
| | | | | | | Socs3, Steap4, Tgm2, Timp1, |
| | | | | | | Tnfrsf12a |
| c6 | 7 | up | E2F1 UP.V1 DN | 0.000 | 0.013 | Ackr3, Adarb1, Angptl4, Cirbp, |
| | | | | | | Cxcl5, Cyp1b1, Fas, Gbp2, Lbp, |
| | | | | | | Lclat1, Neat1, Pdgfra, Rsrp1, |
| | | | | | | Snai1, Stx4a, Tgm2, Zbtb20, Zfr2, |
| | | | | | | Zmynd8 |
| c6 | 7 | up | EGFR UP.V1 UP | 0.000 | 0.002 | Angptl4, Arid5b, Ctgf, Cyp1b1, |
| | | | | | | Enpp1, Ext1, Gbp2, Inhba, Junb, |
| | | | | | | Loxl2, Rnf19a, Smco4, Tnfrsf11b, |
| | | | | | | Wipi1 |
| c6 | 7 | up | ESC J1 UP EARLY.V1 DN | 0.000 | 0.000 | Angptl4, Bloc1s4, Cirbp, Emp2, |
| | | | | | | Gbp2, Id3, Inhbb, Junb, Klf13, |
| | | | | | | Serpine1, Socs3, Tnfrsf12a, Zfp36, |
| | | | | | | Zfp36l1 |
| c6 | 7 | up | ESC J1 UP LATE.V1 DN | 0.000 | 0.030 | Angptl4, Bhlhe40, Cirbp, Dhodh, |
| | | | | | | Mmp11, Pdgfc, Qser1, Socs3 |
| c6 | 7 | up | ESC V6.5 UP EARLY.V1 DN | 0.000 | 0.001 | Aebp1, Axl, Bhlhe40, Ctgf, Emp1, |
| | | | | | | Gbp2, Htra1, Inhba, Inhbb, Junb, |
| | | | | | | Lbp, Lox, Nupr1, S100a6, Scara3, |
| | | | | | | Serpine1, Socs3, Timp2, |
| | | | | | | Tnfrsf12a, Zfp36l1 |
| c6 | 7 | up | ESC V6.5 UP LATE.V1 DN | 0.000 | 0.001 | Angptl4, Emp1, Inhba, Inhbb, Lox, |
| | | | | | | Nupr1, Pdgfc, S100a6, Serpine1, |
| | | | | | | Socs3, Spp1, Timp1 |
| c6 | 7 | up | HINATA NFKB MATRIX | 0.025 | 0.020 | Arhgdia, Serpine1 |
| c6 | 7 | up | HOXA9 DN.V1 DN | 0.005 | 0.036 | Bhlhe40, Fkbp5, Itgb5, Slc29a1, |
| | | | | | | Spp1, Tsc22d3 |
| c6 | 7 | up | IL15 UP.V1 UP | 0.005 | 0.048 | Ecm1, Egr2, Inhbb, Junb, Plec, |
| | | | | | | Spp1, Tnfrsf12a, Tnfsf11 |
| c6 | 7 | up | IL21 UP.V1 DN | 0.005 | 0.041 | Cacna2d3, Cldn10, Cmklr1, |
| | | | | | | Frmd4a, Gdnf, Hkdc1, Htr3b, Kdr, |
| | | | | | | Ldoc1, Mab21l1, Pde1c, Ptgds, |
| | | | | | | Ramp3, Scn7a, Slc16a2, Slc17a1, |
| | | | | | | Timp3, Wnt4, Zfp286, Zfp358 |
| c6 | 7 | up | KRAS.AMP.LUNG UP.V1 DN | 0.000 | 0.029 | Abcb8, Abcd1, Ace, Cd34, Enpp1, |
| | | | | | | Gdf2, Gnat3, Pde11a, Ppm1h, |
| | | | | | | Wisp2 |
| c6 | 7 | up | LEF1 UP.V1 UP | 0.000 | 0.011 | Arc, Axl, Cda, Col6a1, Edar, Egr2, |
| | | | | | | Emp1, Fads1, Hspa1a, Inhbe, |
| | | | | | | Ltbp2, Ntsr1, Plekhf1, Prkaa2, |
| | | | | | | Ptgds, Scml1, Serpine1, Tgm2, |
| | | | | | | Vim, Wnt5a, Wt1, Zfp236, Zfpm2 |
| c6 | 7 | up | MEK UP.V1 DN | 0.005 | 0.015 | 2010111I01Rik, Amigo2, Ascl1, |
| | | | | | | Atp8b1, Fto, Golga4, Id2, Insig2, |
| | | | | | | Jak2, Kif5c, Mafb, Mtcl1, Nmrk1, |
| | | | | | | Nrep, Rhobtb3, Satb2, Sec24d, |
| | | | | | | Sgcg, Slc26a2, Slc35a5, Smpdl3a, |
| | | | | | | Tap1, Tfpi, Top2b, Wisp2 |
| c6 | 7 | up | MTOR UP.V1 DN | 0.000 | 0.019 | Ackr3, Cirbp, Csrp2, Dusp1, |
| | | | | | | Hspa1a, Htra1, Id2, Prss23, Scara3, |
| | | | | | | Tspan4 |
| c6 | 7 | up | PKCA DN.V1 UP | 0.000 | 0.014 | Fmr1nb, Paqr7, Pdk4, Plekhf1, |
| | | | | | | Ptgds, Wisp2, Zfp865 |
| c6 | 7 | up | PRC2 EZH2 UP.V1 UP | 0.005 | 0.036 | Fkbp5, Gas1, Inhbb, Olfml2b, |
| | | | | | | Ptgds, Rnf19a, Sulf1, Tnfsf11 |
| c6 | 7 | up | PTEN DN.V2 UP | 0.000 | 0.015 | Agpat4, Angptl4, Cpa4, Dnajb5, |
| | | | | | | Emp1, Jun, Kcnj3, Klk10, Lipg, |
| | | | | | | Loxl2, Naalad2, Nav3, Nid2, |
| | | | | | | Pcsk7, Plpp3, Ptgds, Rnd1, Tbx5, |
| | | | | | | Thbs4 |
| c6 | 7 | up | RAPA EARLY UP.V1 DN | 0.000 | 0.002 | Axl, Cit, Fto, Glul, Hspa1a, |
| | | | | | | Lgals3bp, Plpp3, Psma7, Snta1, |
| | | | | | | Tspan4 |
| c6 | 7 | up | SNF5 DN.V1 UP | 0.000 | 0.012 | Egr2, Gas1, Id2, Lgals3bp, Medag, |
| | | | | | | Psmb8, Tapbp |
| c6 | 7 | up | STK33 NOMO UP | 0.000 | 0.011 | Arid5b, Btg2, Col6a3, Dusp1, |
| | | | | | | Egr2, Emp1, Hbp1, Jun, Junb, |
| | | | | | | Klf6, Neat1, Zfp36, Zfp36l1 |
| c6 | 7 | up | STK33 SKM UP | 0.000 | 0.006 | Arid5b, Btg2, Cdkn1a, Dusp1, |
| | | | | | | Fam177a, Fam20c, Gbp2, Junb, |
| | | | | | | Klf6, Lmbrd2, Mafb, Rnf19a, |
| | | | | | | Rnf19b, Tnfrsf11b, Zfp36 |
| c6 | 7 | up | STK33 UP | 0.000 | 0.000 | Arid5b, Btg2, Cdkn1a, Col6a3, |
| | | | | | | Dusp1, Egr2, Emp1, Fam177a, |
| | | | | | | Fam20c, Hbp1, Jun, Junb, Klf6, |
| | | | | | | Lmbrd2, Mafb, Rnf19a, Rnf19b, |
| | | | | | | Zfp36, Zfp36l1 |
| c6 | 7 | up | TBK1.DN.48HRS DN | 0.004 | 0.004 | Atp6v1d, Bphl, Cryz, Gale, |
| | | | | | | Hspa1a, Il13ral, Mapk9, Pdgfa, |
| | | | | | | Psmb8, Terf2ip |
| c6 | 7 | up | TGFB UP.V1 UP | 0.000 | 0.010 | Bhlhe40, Ctgf, Id3, Jun, Junb, |
| | | | | | | Ltbp2, Mafb, Rnf19b, Snai1, |
| | | | | | | Wnt4, Zfp36l1 |
| c6 | 7 | up | VEGF A UP.V1 UP | 0.000 | 0.014 | Adarb1, Ddit4, Ecm1, Enpp2, Id2, |
| | | | | | | Psmb8 |
| c6 | 8 | down | HINATA NFKB IMMU INF | 0.007 | 0.008 | Ccl20, Irf1, Nfkbia |
| c6 | 8 | up | ALK DN.V1 UP | 0.000 | 0.004 | Angptl4, Ddit4, Fmo1, Mt1, Spp1, |
| | | | | | | Vstm4 |
| c6 | 8 | up | E2F1 UP.V1 DN | 0.000 | 0.009 | Adamts1, Adarb1, Angptl4, Cirbp, |
| | | | | | | Cyp1b1, Dok1, Gadd45a, Lama4, |
| | | | | | | Rsrp1, Stat3, Synpo, Tgm2 |
| c6 | 8 | up | ESC V6.5 UP LATE.V1 DN | 0.000 | 0.030 | Angptl4, Cbx7, Gadd45a, Inhbb, |
| | | | | | | Lgals3, Lox, Serpine1, Spp1, |
| | | | | | | Timp1 |
| c6 | 8 | up | P53 DN.V1 DN | 0.003 | 0.039 | Btg2, Ecm1, Fbln5, Gpnmb, Spp1, |
| | | | | | | St3gal6 |
| h | 0 | down | HALLMARK ADIPOGENESIS | 0.003 | 0.024 | Angptl4, Apoe, Cd36, Cyp4b1, |
| | | | | | | Dnajb9, Gadd45a, Lifr, Mylk, |
| | | | | | | Pim3, Pparg, Sult1a1, Tob1, Ubc |
| h | 0 | down | HALLMARK G2M | 0.006 | 0.020 | Ccnt1, Efna5, Hif1a, Prpf4b, Sfpq, |
| | | | CHECKPOINT | | | Top1, Tra2b, Ythdc1 |
| h | 0 | down | HALLMARK HYPOXIA | 0.015 | 0.021 | Akap12, Aldob, Angptl4, Btg1, |
| | | | | | | Chst2, Cited2, Fos, Hspa5, Ier3, |
| | | | | | | Jun, Klf6, Lalba, Maff, Nfil3, |
| | | | | | | Noct, Nr3c1, Pdgfb, Pim1, Pnrc1, |
| | | | | | | Ppp1r15a, Siah2, Tiparp, Wsb1, |
| | | | | | | Zfp36 |
| h | 0 | down | HALLMARK INFLAMMATORY | 0.017 | 0.038 | Aplnr, Axl, Btg2, Chst2, Csf1, |
| | | | RESPONSE | | | Hif1a, Klf6, Nfkbia, Pde4b, Ptger2, |
| | | | | | | Rnf144b, Sele |
| h | 0 | down | HALLMARK TNFA SIGNALING | 0.000 | 0.000 | Birc3, Bmp2, Btg1, Btg2, Ccnl1, |
| | | | VIA NFKB | | | Cebpb, Csf1, Egr1, Fos, Fosb, |
| | | | | | | Gadd45a, Id2, Ier3, Il6st, Jun, |
| | | | | | | Junb, Kdm6b, Klf6, Maff, Nfil3, |
| | | | | | | Nfkbia, Plpp3, Pnrc1, Ppp1r15a, |
| | | | | | | Ptx3, Rhob, Sat1, Sik1, Tiparp, |
| | | | | | | Tnfaip2, Tnip1, Trib1, Zfp36 |
| h | 0 | down | HALLMARK UV RESPONSE DN | 0.006 | 0.023 | Cited2, Dlc1, Id1, Igf1r, Map1b, |
| | | | | | | Nr3c1, Plpp3, Pparg, Rgs4, |
| | | | | | | Sfmbt1, Tfpi, Tgfbr3, Ythdc1 |
| h | 0 | down | HALLMARK UV RESPONSE UP | 0.000 | 0.015 | Bmp2, Btg1, Btg2, Chka, Ctsl, |
| | | | | | | Dnajb1, Fos, Fosb, Il6st, Junb, |
| | | | | | | Nfkbia, Rhob, Sqstm1, Sult1a1 |
| h | 0 | up | HALLMARK ANGIOGENESIS | 0.000 | 0.000 | App, Ccnd2, Slco2a1, Stc1 |
| h | 11 | up | HALLMARK ANDROGEN | 0.000 | 0.040 | Actn1, Fkbp5, Plpp1, Sat1, Sgk1 |
| | | | RESPONSE | | | |
| h | 12 | down | HALLMARK EPITHELIAL | 0.006 | 0.029 | Cap2, Cdh2, Col5a3, Col6a2, |
| | | | MESENCHYMAL TRANSITION | | | Col6a3, Foxc2, Gadd45b, Gas1, |
| | | | | | | Gem, Pvr, Sat1, Serpinh1, Sgcb, |
| | | | | | | Sntb1, Thyl, Tnfrsf12a |
| h | 12 | down | HALLMARK G2M | 0.000 | 0.010 | Atf5, Cdkn2c, Hoxc10, Hspa8, |
| | | | CHECKPOINT | | | Marcks, Notch2, Odf2, Sqle, |
| | | | | | | Srsf10, Tra2b |
| h | 12 | down | HALLMARK MYOGENESIS | 0.006 | 0.043 | Akt2, Ckb, Col6a2, Col6a3, Cryab, |
| | | | | | | Eif4a2, Flii, Gadd45b, Ifrd1, Igf1, |
| | | | | | | Mef2c, Mef2d, Myh3, Myl1, |
| | | | | | | Notch1, Pygm, Speg |
| h | 12 | down | HALLMARK TNFA SIGNALING | 0.000 | 0.012 | Atf3, Btg1, Egr1, Fosb, Gadd45b, |
| | | | VIA NFKB | | | Gem, Ier5, Irf1, Kdm6b, Klf4, |
| | | | | | | Marcks, Nfil3, NEkbia, Pde4b, |
| | | | | | | Phlda1, Sat1, Tiparp, Tnfaip2 |
| h | 12 | down | HALLMARK UV RESPONSE UP | 0.003 | 0.039 | Atf3, Btg1, Cck, Fosb, Irf1, |
| | | | | | | Nfkbia, Rrad, Shox2 |
| h | 1 | down | HALLMARK APICAL | 0.004 | 0.035 | Akt3, Cdh11, Cldn18, Fscn1, |
| | | | JUNCTION | | | Icam1, Mdk, Vcam1 |
| h | 1 | down | HALLMARK G2M | 0.000 | 0.005 | Hspa8, Ilf3, Mt2, Myc, Notch2, |
| | | | CHECKPOINT | | | Prpf4b, Top1, Tra2b |
| h | 1 | down | HALLMARK MYC TARGETS V1 | 0.000 | 0.007 | Cnbp, Eif4h, Fam120a, Ifrd1, Myc, |
| | | | | | | Pgk1, Prdx4, Psma7, Psmd1, Srsf7, |
| | | | | | | Tra2b |
| h | 1 | down | HALLMARK TNFA SIGNALING | 0.000 | 0.018 | Ccl2, Ccnl1, Cebpb, Csf1, Cxcl1, |
| | | | VIA NFKB | | | Fosb, Icam1, Irf1, Kdm6b, Klf2, |
| | | | | | | Klf6, Myc, Nfat5, Nfkbia, |
| | | | | | | Ppp1r15a, Rhob, Sat1, Sgk1, |
| | | | | | | Socs3, Tiparp, Tnc, Trib1, Zfp36 |
| h | 1 | down | HALLMARK UV RESPONSE UP | 0.006 | 0.020 | Cltb, Cxcl1, Dnajb1, Fosb, Icam1, |
| | | | | | | Irf1, Nfkbia, Rhob, Rrad |
| h | 1 | up | HALLMARK APOPTOSIS | 0.000 | 0.023 | Cdkn1a, Ctnnb1, Gadd45a, Gpx3, |
| | | | | | | H1f0, Ier3, Tap1, Timp1, Timp2, |
| | | | | | | Tnfrsf12a, Txnip |
| h | 1 | up | HALLMARK COAGULATION | 0.000 | 0.000 | C1s2, Crip2, Ctsb, Ctsl, Fbn1, |
| | | | | | | Lrp1, Mmp11, Pros1, Prss23, |
| | | | | | | Seipine1, Serping1, Timp1, Trf |
| h | 1 | up | HALLMARK GLYCOLYSIS | 0.002 | 0.021 | Ddit4, Eno1, Ext2, Ier3, Igfbp3, |
| | | | | | | Mif, Pgam1, Polr3k, Tpi1, Vegfa |
| h | 1 | up | HALLMARK HYPOXIA | 0.000 | 0.004 | Adm, Anxa2, Bhlhe40, Cdkn1a, |
| | | | | | | Csrp2, Ddit4, Eno1, Errfi1, Ier3, |
| | | | | | | Igfbp3, Lox, Mif, Pfkl, Sdc4, |
| | | | | | | Selenbp1, Serpine1, Tgm2, Tpi1, |
| | | | | | | Vegfa, Wisp2 |
| h | 1 | up | HALLMARK MYOGENESIS | 0.000 | 0.020 | Aebp1, Bhlhe40, Cdkn1a, Dapk2, |
| | | | | | | Gpx3, Igfbp3, Itgb5, Ptgis |
| h | 1 | up | HALLMARK P53 PATHWAY | 0.002 | 0.036 | Ankra2, Cd82, Cdkn1a, Cgrrf1, |
| | | | | | | Ddit4, Gadd45a, Hdac3, Ier3, |
| | | | | | | Inhbb, Nol8, Nupr1, Rab40c, |
| | | | | | | Rad9a, Sp1, Tap1, Txnip, Vdr, |
| | | | | | | Wrap73 |
| h | 2 | down | HALLMARK ESTROGEN | 0.000 | 0.002 | Abhd2, Bag1, Btg3, Cox6c, Emp2, |
| | | | RESPONSE LATE | | | Fos, Id2, Impa2, Jak1, Klf4, Nab2, |
| | | | | | | Nrip1, Pcp4, Scarb1, Siah2, |
| | | | | | | Slc26a2, Slc29a1, Tob1, Xbp1 |
| h | 2 | down | HALLMARK KRAS SIGNALING | 0.003 | 0.038 | Ank, Dusp6, Emp1, F13a1, |
| | | | UP | | | Gadd45g, Id2, Klf4, Pcp4, |
| | | | | | | Ppp1r15a, Scn1b, Torlaip2, Trib1 |
| h | 2 | down | HALLMARK TNFA SIGNALING | 0.000 | 0.000 | Atf3, Atp2b1, B4galt1, Bhlhe40, |
| | | | VIA NFKB | | | Btg1, Btg3, Ccnl1, Cdkn1a, Cebpb, |
| | | | | | | F3, Fos, Fosb, Gadd45b, Gem, |
| | | | | | | Hes1, Id2, Ier3, Ier5, Kdm6b, |
| | | | | | | Klf10, Klf4, Klf9, Ninj1, Nr4a2, |
| | | | | | | Pde4b, Phlda1, Ppp1r15a, Rcan1, |
| | | | | | | Rela, Sat1, Sdc4, Socs3, Tiparp, |
| | | | | | | Tnip1, Trib1, Vegfa |
| h | 3 | down | HALLMARK EPITHELIAL | 0.000 | 0.002 | Abi3bp, Bgn, Col1a1, Col1a2, |
| | | | MESENCHYMAL TRANSITION | | | Col3a1, Col5a2, Col6a2, Col6a3, |
| | | | | | | Col8a2, Comp, Ecm2, Emp3, |
| | | | | | | Gadd45b, Gas1, Id2, Msx1, Pcolce, |
| | | | | | | Pcolce2, Plod2, Pmepa1, Pmp22, |
| | | | | | | Rhob, Serpinh1, Sparc, Timp3, |
| | | | | | | Tnfrsf11b, Tnfrsf12a, Vcan |
| h | 6 | down | HALLMARK APOPTOSIS | 0.000 | 0.000 | App, Atf3, Btg2, Cdkn1a, Ddit3, |
| | | | | | | Dnaja1, Gadd45b, Hspb1, Ier3, |
| | | | | | | Ifitm3, Irf1, Jun, Mcl1, Nedd9, |
| | | | | | | Pdcd4, Rhob, Sat1, Slc20a1, |
| | | | | | | Tgfb2, Timp3, Tnfsf10 |
| h | 6 | down | HALLMARK E2F TARGETS | 0.000 | 0.032 | Cdkn1a, Cit, Prdx4, Smc6, Srsf2, |
| | | | | | | Tra2b |
| h | 6 | down | HALLMARK ESTROGEN | 0.013 | 0.042 | Ckb, Emp2, Fos, Id2, Klf4, Mettl3, |
| | | | RESPONSE LATE | | | Pdcd4, Sgk1, St6galnac2, Tob1, |
| | | | | | | Tspan13, Zfp36 |
| h | 6 | down | HALLMARK IL6 JAK STAT3 | 0.002 | 0.002 | Acvrl1, Cxcl1, Irf1, Irf9, Jun, |
| | | | SIGNALING | | | Osmr, Socs1, Socs3 |
| h | 6 | down | HALLMARK INTERFERON | 0.000 | 0.000 | B2m, Bst2, Herc6, Ifitm3, Irf1, |
| | | | ALPHA RESPONSE | | | Irf2, Irf7, Irf9, Isg15, Lgals3bp, |
| | | | | | | Plscr1, Psmb8, Psmb9, Rsad2, |
| | | | | | | Trim5 |
| h | 6 | down | HALLMARK INTERFERON | 0.000 | 0.000 | Arid5b, B2m, Bst2, Btg1, Cdkn1a, |
| | | | GAMMA RESPONSE | | | Herc6, Ifitm3, Irf1, Irf2, Irf7, Irf9, |
| | | | | | | Isg15, Lgals3bp, Lysmd2, Nfkbia, |
| | | | | | | Nod1, Plscr1, Psmb10, Psmb8, |
| | | | | | | Psmb9, Rsad2, Socs1, Socs3, |
| | | | | | | Tapbp, Tnfsf10, Tor1b, Vcam1, |
| | | | | | | Xaf1 |
| h | 6 | down | HALLMARK KRAS SIGNALING | 0.009 | 0.034 | Btg2, Copz2, Edn1, Efhd1, Kcne2, |
| | | | DN | | | Magix, Rsad2, Sgk1, Skil, Tgfb2 |
| h | 6 | down | HALLMARK MYC TARGETS VI | 0.004 | 0.019 | Eif4e, Ifrd1, Prdx4, Ptges3, Srsf2, |
| | | | | | | Srsf7, Ssb, Tra2b |
| h | 6 | down | HALLMARK P53 PATHWAY | 0.000 | 0.023 | App, Atf3, Btg1, Btg2, Cd81, |
| | | | | | | Cdkn1a, Ddit3, Ephx1, Eps8l2, |
| | | | | | | Fos, Hexim1, Ier3, Ier5, Jun, Klf4, |
| | | | | | | Ppp1r15a, Sat1, Socs1, Sphk1, |
| | | | | | | Tm4sf1, Tob1, Zfp36l1 |
| h | 6 | down | HALLMARK PI3K AKT MTOR | 0.021 | 0.048 | Cdkn1a, Ddit3, Eif4e, Nck1, Nod1, |
| | | | SIGNALING | | | Pik3r3, Plcb1 |
| h | 6 | down | HALLMARK TNFA SIGNALING | 0.000 | 0.000 | Atf3, Btg1, Btg2, Ccnl1, Cdkn1a, |
| | | | VIA NFKB | | | Cxcl1, Cyr61, Dusp1, Edn1, Egr1, |
| | | | | | | Fos, Fosb, Gadd45b, Hes1, Id2, |
| | | | | | | Ier2, Ier3, Ier5, Irf1, Jag1, Jun, |
| | | | | | | Junb, Kdm6b, Klf2, Klf4, Klf6, |
| | | | | | | Mcl1, Nfkbia, Nr4a1, Plpp3, |
| | | | | | | Pnrc1, Ppp1r15a, Rhob, Sat1, |
| | | | | | | Sgk1, Socs3, Sphk1, Zfp36 |
| h | 6 | down | HALLMARK UNFOLDED | 0.026 | 0.031 | Atf3, Atf4, Cnot6, Dctn1, Dnajb9, |
| | | | PROTEIN RESPONSE | | | Eif4e, Fus, Hspa5, Pdia6 |
| h | 6 | down | HALLMARK UV RESPONSE UP | 0.000 | 0.000 | Amd1, Atf3, Btg1, Btg2, Cdkn1c, |
| | | | | | | Chka, Cxcl1, Dnaja1, Dnajb1, |
| | | | | | | Ephxl, Fos, Fosb, Igfbp2, Irf1, |
| | | | | | | Junb, Msx1, Nfkbia, Nr4a1, Rhob |
| h | 7 | up | HALLMARK ALLOGRAFT | 0.000 | 0.000 | Capg, Cxcl9, Gbp2, Ifngr1, Inhba, |
| | | | REJECTION | | | Inhbb, Stat1, Tapbp, Timp1 |
| h | 7 | up | HALLMARK ANDROGEN | 0.000 | 0.001 | Acsl3, Akt1, Ank, Arid5b, Ccnd1, |
| | | | RESPONSE | | | Fads1, Fkbp5, Idi1, Itgav, Sec24d, |
| | | | | | | Slc26a2, Slc38a2, Steap4 |
| h | 7 | up | HALLMARK APOPTOSIS | 0.001 | 0.006 | Ank, Btg2, Cdkn1a, Emp1, Ifngr1, |
| | | | | | | Jun, Pdgfrb, Timp1, Timp2, |
| | | | | | | Timp3, Tnfrsf12a |
| h | 7 | up | HALLMARK COAGULATION | 0.000 | 0.003 | Adam9, Htra1, Lrp1, Mmp11, |
| | | | | | | Prss23, Serpine1, Serping1, Timp1, |
| | | | | | | Timp3 |
| h | 7 | up | HALLMARK COMPLEMENT | 0.001 | 0.006 | Adam9, Gnb4, Hspa1a, Lrp1, |
| | | | | | | Serpine1, Serping1, Timp1, Timp2 |
| h | 7 | up | HALLMARK EPITHELIAL | 0.000 | 0.006 | Capg, Col12a1, Col6a3, Ctgf, |
| | | | MESENCHYMAL TRANSITION | | | Ecm1, Gas1, Htra1, Id2, Inhba, |
| | | | | | | Itgav, Itgb5, Jun, Lox, Loxl2, Lrp1, |
| | | | | | | Pdgfrb, Qsox1, Sdc1, Serpine1, |
| | | | | | | Spp1, Tgm2, Timp1, Timp3, |
| | | | | | | Tnfrsf11b, Tnfrsf12a, Vim |
| h | 7 | up | HALLMARK GLYCOLYSIS | 0.004 | 0.030 | Angptl4, Cited2, Ddit4, Dsc2, |
| | | | | | | Ext1, Il3ra1, Mif, Qsox1, Sdc1 |
| h | 7 | up | HALLMARK HYPOXIA | 0.000 | 0.000 | Ackr3, Angptl4, Bhlhe40, Btg1, |
| | | | | | | Cdkn1a, Cited2, Csrp2, Ctgf, |
| | | | | | | Ddit4, Dusp1, Ext1, Jun, Klf6, |
| | | | | | | Lox, Mif, Pgf, Scarb1, Serpine1, |
| | | | | | | Tgm2, Wisp2, Zfp36 |
| h | 7 | up | HALLMARK IL2 STAT5 | 0.000 | 0.000 | Bhlhe40, Capg, Col6a1, Ecm1, |
| | | | SIGNALING | | | Emp1, Enpp1, Ifngr1, Itgav, Klf6, |
| | | | | | | Lclat1, Plec, Spp1, Tgm2, Tnfsf11 |
| h | 7 | up | HALLMARK IL6 JAK STAT3 | 0.000 | 0.000 | Cxcl9, Ifngr1, Il13ra1, Il9r, Jun, |
| | | | SIGNALING | | | Pdgfc, Socs3, Stat1, Tnfrsf12a |
| h | 7 | up | HALLMARK INFLAMMATORY | 0.005 | 0.017 | Axl, Btg2, Cdkn1a, Cxcl9, Inhba, |
| | | | RESPONSE | | | Klf6, Serpine1, Tapbp, Timp1 |
| h | 7 | up | HALLMARK INTERFERON | 0.000 | 0.001 | Arid5b, Btg1, Cdkn1a, Cxcl9, Fas, |
| | | | GAMMA RESPONSE | | | Isoc1, Lgals3bp, Psmb8, Serping1, |
| | | | | | | Socs3, Stat1, Tapbp, Trim21, Xaf1 |
| h | 7 | up | HALLMARK KRAS SIGNALING | 0.000 | 0.006 | Ace, Angptl4, Ank, Emp1, Id2, |
| | | | UP | | | Inhba, Mafb, Mmp11, Psmb8, |
| | | | | | | Spp1 |
| h | 7 | up | HALLMARK P53 PATHWAY | 0.001 | 0.004 | Btg1, Btg2, Cdkn1a, Ddit4, Inhbb, |
| | | | | | | Jun, Ldhb, Nupr1, Rnf19b, Sdc1, |
| | | | | | | Zfp36l1 |
| h | 7 | up | HALLMARK TGF BETA | 0.026 | 0.044 | Id2, Id3, Junb, Ltbp2, Nog, |
| | | | SIGNALING | | | Serpine1 |
| h | 7 | up | HALLMARK TNFA SIGNALING | 0.000 | 0.002 | Ackr3, Bhlhe40, Btg1, Btg2, |
| | | | VIA NFKB | | | Cdkn1a, Dusp1, Egr2, Id2, Inhba, |
| | | | | | | Jun, Junb, Klf6, Klf9, Plpp3, |
| | | | | | | Rnf19b, Serpine1, Socs3, Zfp36 |

TABLE 8

| Collect | Cluster | Enrich | Set | pval | qval | Genes |
|---|---|---|---|---|---|---|
| c2 | 1 | down | PID BMP PATHWAY | 0 | 0.027074417 | Bambi, Bmp4, Chrdl1, |
| | | | | | | Fst, Grem1, Ppp1r15a |
| c2 | 1 | down | REACTOME | 0 | 0.0290 | Adipoq, Angpt14, |
| | | | TRANSCRIPTIONAL | | | Cebpb, Ebf1, Lpl, |
| | | | REGULATION OF | | | Med22, Tgs1 |
| | | | WHITE ADIPOCYTE | | | |
| | | | DIFFERENTIATION | | | |
| c2 | 1 | down | KEGG PPAR | 0 | 0.0221 | Acox1, Adipoq, |
| | | | SIGNALING | | | Angptl4, Gk2, Lpl |
| | | | PATHWAY | | | |
| c2 | 1 | up | REACTOME | 0 | 4.53E−04 | Mmp11, Timp1, Timp2 |
| | | | DEGRADATION OF | | | |
| | | | THE | | | |
| | | | EXTRACELLULAR | | | |
| | | | MATRIX | | | |
| h | 1 | up | HALLMARK HYPO | 0 | 0.0040 | Adm, Anxa2, Bhlhe40, |
| | | | XIA | | | Cdkn1a, Csrp2, Ddit4, |
| | | | | | | Eno1, Errfi1, Ier3, |
| | | | | | | Igfbp3, Lox, Mif, Pfkl, |
| | | | | | | Sdc4, Selenbp1, |
| | | | | | | Serpine1, Tgm2, Tpi1, |
| | | | | | | Vegfa, Wisp2 |
| c2 | 7 | up | NABA ECM | 0 | 0.0406 | Adam9, Fam20a, |
| | | | REGULATORS | | | Fam20c, Hpse2, Htra1, |
| | | | | | | Htra3, Kng2, Lox, |
| | | | | | | Loxl2, Mmp11, |
| | | | | | | Pappa2, Serpine1, |
| | | | | | | Serping1, Sulf1, Tgm2, |
| | | | | | | Timp1, Timp2, Timp3 |

### Example 11—Leukemia Broadly Impairs Normal Hematopoiesis-Regulatory Expression

The changes in the stroma may contribute to altered support of normal blood cell growth, through deregulation of the expression of key HSC niche factors, especially Cxcl12 and Kitl across MSCs, aBMECs, the earliest OLCs (sub-cluster 7-0) and EC progenitors (FIGS. 13H-131); conversely, Cxcl12, Kitl and Angpt1 were upregulated in Fibroblast-Is (FIG. 13J). These findings are consistent with prior observations of AML reducing HSC persistence and localization to the bone marrow (Zhang et al., 2012), and that Cxcl12-secreting-CAFs (similar to Fibroblast-is) are associated with a cancer promoting phenotype in breast cancer (Ahirwar et al., 2018; Costa et al., 2018).

Among other factors, Spp1, a negative regulator of HSC pool size (Stier et al., 2005) and HSC proliferation (Nilsson et al., 2005), which is correlated with poor prognosis in AML patients (Chen et al., 2017), was upregulated in MSCs, OLCs, ECs, fibroblasts and pericytes (FIGS. 13H and 14F). MSCs down regulated other HSC niche factors including Angpt1, an agonist of Tek receptor expressed on ECs and HSCs (Arai et al., 2004) (and upregulated its antagonist Angpt2 (Gomei et al., 2010)); factors that promote lymphoid (117) or myeloid (Csf1) differentiation (Muegge et al., 1993; Namen et al., 1988) (Mossadegh-Keller et al., 2013) and Vcam1, a regulator of HSC homing to bone marrow (Papayannopoulou et al., 1995) (FIG. 13H). Thus, the osteogenic differentiation block induced by leukemia in MSCs and OLCs, was further accompanied by a loss of HSC niche factors production by multiple cell types.

### REFERENCES

Abdallah, B. M., Al-Shammary, A., Skagen, P., Abu Dawud, R., Adjaye, J., Aldahmash, A., and Kassem, M. (2015). CD34 defines an osteoprogenitor cell population in mouse bone marrow stromal cells. Stem cell research 15, 449-458.
Acar, M., Kocherlakota, K. S., Murphy, M. M., Peyer, J. G., Oguro, H., Inra, C. N., Jaiyeola, C., Zhao, Z., Luby-Phelps, K., and Morrison, S. J. (2015). Deep imaging of bone marrow shows non-dividing stem cells are mainly perisinusoidal. Nature 526, 126-130.
Ahirwar, D. K., Nasser, M. W., Ouseph, M. M., Elbaz, M., Cuitino, M. C., Kladney, R. D., Varikuti, S., Kaul, K., Satoskar, A. R., Ramaswamy, B., et al. (2018). Fibroblast-derived CXCL12 promotes breast cancer metastasis by facilitating tumor cell intravasation. Oncogene.
Angerer, P., Haghverdi, L., Buttner, M., Theis, F. J., Marr, C., and Buettner, F. (2016). destiny: diffusion maps for large-scale single-cell data in R. Bioinformatics 32, 1241-1243.
Arai, F., Hirao, A., Ohmura, M., Sato, H., Matsuoka, S., Takubo, K., Ito, K., Koh, G. Y., and Suda, T. (2004). Tie2/angiopoietin-1 signaling regulates hematopoietic stem cell quiescence in the bone marrow niche. Cell 118, 149-161.
Armulik, A., Genove, G., and Betsholtz, C. (2011). Pericytes: developmental, physiological, and pathological perspectives, problems, and promises. Developmental cell 21, 193-215.
Arnold, M. A., Kim, Y., Czubryt, M. P., Phan, D., McAnally, J., Qi, X., Shelton, J. M., Richardson, J. A., Bassel-Duby, R., and Olson, E. N. (2007). MEF2C transcription factor controls chondrocyte hypertrophy and bone development. Developmental cell 12, 377-389.
Arranz, L., Sanchez-Aguilera, A., Martin-Perez, D., Isern, J., Langa, X., Tzankov, A., Lundberg, P., Muntion, S., Tzeng, Y. S., Lai, D. M., et al. (2014). Neuropathy of haematopoietic stem cell niche is essential for myeloproliferative neoplasms. Nature 512, 78-81.
Asahara, T., Murohara, T., Sullivan, A., Silver, M., van der Zee, R., Li, T., Witzenbichler, B., Schatteman, G., and Isner, J. M. (1997). Isolation of putative progenitor endothelial cells for angiogenesis. Science 275, 964-967.
Bastian, M., Heymann, S., and Jacomy, M. (2009). Gephi: an open source software for exploring and manipulating networks. Icwsm 8, 361-362.
Bauche, I. B., El Mkadem, S. A., Pottier, A. M., Senou, M., Many, M. C., Rezsohazy, R., Penicaud, L., Maeda, N., Funahashi, T., and Brichard, S. M. (2007). Overexpression of adiponectin targeted to adipose tissue in transgenic mice: impaired adipocyte differentiation. Endocrinology 148, 1539-1549.
Benjamini, Y., and Hochberg, Y. (1995). Controlling the false discovery rate: a practical and powerful approach to multiple testing. Journal of the royal statistical society Series B (Methodological), 289-300.
Bernad, A., Kopf, M., Kulbacki, R., Weich, N., Koehler, G., and Gutierrez-Ramos, J. C. (1994). Interleukin-6 is required in vivo for the regulation of stem cells and committed progenitors of the hematopoietic system. Immunity 1, 725-731.
Beygelzimer, A., Kakadet, S., Langford, J., Arya, S., Mount, D., Li, S., and Li, M. S. (2015). Package ‘FNN’. Accessed June 1.
Blondel, V. D., Guillaume, J.-L., Lambiotte, R., and Lefebvre, E. (2008). Fast unfolding of communities in large networks. Journal of statistical mechanics: theory and experiment 2008, P10008.
Boyd, A. L., Reid, J. C., Salci, K. R., Aslostovar, L., Benoit, Y. D., Shapovalova, Z., Nakanishi, M., Porras, D. P., Almakadi, M., Campbell, C. J. V., et al. (2017). Acute myeloid leukaemia disrupts endogenous myelo-erythropoiesis by compromising the adipocyte bone marrow niche. Nature cell biology 19, 1336-1347.
Breitbach, M., Kimura, K., Luis, T. C., Fuegemann, C. J., Woll, P. S., Hesse, M., Facchini, R., Rieck, S., Jobin, K., Reinhardt, J., et al. (2018). In Vivo Labeling by CD73 Marks Multipotent Stromal Cells and Highlights Endothelial Heterogeneity in the Bone Marrow Niche. Cell stem cell 22, 262-276 e267.
Butler, J. M., Nolan, D. J., Vertes, E. L., Varnum-Finney, B., Kobayashi, H., Hooper, A. T., Seandel, M., Shido, K., White, I. A., Kobayashi, M., et al. (2010). Endothelial cells are essential for the self-renewal and repopulation of Notch-dependent hematopoietic stem cells. Cell stem cell 6, 251-264.
Calvi, L. M., Adams, G. B., Weibrecht, K. W., Weber, J. M., Olson, D. P., Knight, M. C., Martin, R. P., Schipani, E., Divieti, P., Bringhurst, F. R., et al. (2003). Osteoblastic cells regulate the haematopoietic stem cell niche. Nature 425, 841-846.
Chan, C. K., Seo, E. Y., Chen, J. Y., Lo, D., McArdle, A., Sinha, R., Tevlin, R., Seita, J., Vincent-Tompkins, J., Wearda, T., et al. (2015). Identification and specification of the mouse skeletal stem cell. Cell 160, 285-298.
Chan, D., Cole, W. G., Chow, C. W., Mundlos, S., and Bateman, J. F. (1995). A COL2A1 mutation in achondrogenesis type II results in the replacement of type II collagen by type I and III collagens in cartilage. The Journal of biological chemistry 270, 1747-1753.
Chen, Y. B., Ren, S. M., Li, S. D., and Du, Z. (2017). Prognostic significance of osteopontin in acute myeloid leukemia: A meta-analysis. Molecular and clinical oncology 7, 275-280.
Corada, M., Liao, F., Lindgren, M., Lampugnani, M. G., Breviario, F., Frank, R., Muller, W. A., Hicklin, D. J., Bohlen, P., and Dejana, E. (2001). Monoclonal antibodies directed to different regions of vascular endothelial cadherin extracellular domain affect adhesion and clustering of the protein and modulate endothelial permeability. Blood 97, 1679-1684.
Corral, J., Lavenir, I., Impey, H., Warren, A. J., Forster, A., Larson, T. A., Bell, S., McKenzie, A. N., King, G., and Rabbitts, T. H. (1996). An Mll-AF9 fusion gene made by homologous recombination causes acute leukemia in chimeric mice: a method to create fusion oncogenes. Cell 85, 853-861.
Costa, A., Kieffer, Y., Scholer-Dahirel, A., Pelon, F., Bourachot, B., Cardon, M., Sirven, P., Magagna, I., Fuhrmann, L., Bernard, C., et al. (2018). Fibroblast Heterogeneity and Immunosuppressive Environment in Human Breast Cancer. Cancer cell 33, 463-479 e410.
Coutu, D. L., Kokkaliaris, K. D., Kunz, L., and Schroeder, T. (2017). Three-dimensional map of nonhematopoietic bone and bone-marrow cells and molecules. Nature biotechnology 35, 1202-1210.
Delmas, P. D., Demiaux, B., Malaval, L., Chapuy, M. C., and Meunier, P. J. (1986). [Osteocalcin (or bone gla-protein), a new biological marker for studying bone pathology]. Presse medicale 15, 643-646.
Ding, L., and Morrison, S. J. (2013). Haematopoietic stem cells and early lymphoid progenitors occupy distinct bone marrow niches. Nature 495, 231-235.
Ding, L., Saunders, T. L., Enikolopov, G., and Morrison, S. J. (2012). Endothelial and perivascular cells maintain haematopoietic stem cells. Nature 481, 457-462.
Doan, P. L., Russell, J. L., Himburg, H. A., Helms, K., Harris, J. R., Lucas, J., Holshausen, K. C., Meadows, S. K., Daher, P., Jeffords, L. B., et al. (2013). Tie2(+) bone marrow endothelial cells regulate hematopoietic stem cell regeneration following radiation injury. Stem cells 31, 327-337.
Doege, K. J., Sasaki, M., Kimura, T., and Yamada, Y. (1991). Complete coding sequence and deduced primary structure of the human cartilage large aggregating proteoglycan, aggrecan. Human-specific repeats, and additional alternatively spliced forms. The Journal of biological chemistry 266, 894-902.
Dominici, M., Le Blanc, K., Mueller, I., Slaper-Cortenbach, I., Marini, F., Krause, D., Deans, R., Keating, A., Prockop, D., and Horwitz, E. (2006). Minimal criteria for defining multipotent mesenchymal stromal cells. The International Society for Cellular Therapy position statement. Cytotherapy 8, 315-317.
Dong, L., Yu, W. M., Zheng, H., Loh, M. L., Bunting, S. T., Pauly, M., Huang, G., Zhou, M., Broxmeyer, H. E., Scadden, D. T., et al. (2016). Leukaemogenic effects of Ptpnl 1 activating mutations in the stem cell microenvironment. Nature 539, 304-308.
Duarte, D., Hawkins, E. D., Akinduro, O., Ang, H., De Filippo, K., Kong, I. Y., Haltalli, M., Ruivo, N., Straszkowski, L., Vervoort, S. J., et al. (2018). Inhibition of Endosteal Vascular Niche Remodeling Rescues Hematopoietic Stem Cell Loss in AML. Cell stem cell 22, 64-77 e66.
Foster, J. W., Dominguez-Steglich, M. A., Guioli, S., Kwok, C., Weller, P. A., Stevanovic, M., Weissenbach, J., Mansour, S., Young, I. D., Goodfellow, P. N., et al. (1994). Campomelic dysplasia and autosomal sex reversal caused by mutations in an SRY-related gene. Nature 372, 525-530.
Frisch, B. J., Ashton, J. M., Xing, L., Becker, M. W., Jordan, C. T., and Calvi, L. M. (2012). Functional inhibition of osteoblastic cells in an in vivo mouse model of myeloid leukemia. Blood 119, 540-550.
Fuady, J. H., Bordoli, M. R., Abreu-Rodriguez, I., Kristiansen, G., Hoogewijs, D., Stiehl, D. P., and Wenger, R. H. (2014). Hypoxia-inducible factor-mediated induction of WISP-2 contributes to attenuated progression of breast cancer. Hypoxia 2, 23-33.
Gelse, K., Poschl, E., and Aigner, T. (2003). Collagens—structure, function, and biosynthesis. Advanced drug delivery reviews 55, 1531-1546.
Geyh, S., Rodriguez-Paredes, M., Jager, P., Khandanpour, C., Cadeddu, R. P., Gutekunst, J., Wilk, C. M., Fenk, R., Zilkens, C., Hermsen, D., et al. (2016). Functional inhibition of mesenchymal stromal cells in acute myeloid leukemia. Leukemia 30, 683-691.
Gomei, Y., Nakamura, Y., Yoshihara, H., Hosokawa, K., Iwasaki, H., Suda, T., and Arai, F. (2010). Functional differences between two Tie2 ligands, angiopoietin-1 and -2, in regulation of adult bone marrow hematopoietic stem cells. Exp Hematol 38, 82-89.
Greenbaum, A., Hsu, Y. M., Day, R. B., Schuettpelz, L. G., Christopher, M. J., Borgerding, J. N., Nagasawa, T., and Link, D. C. (2013). CXCL12 in early mesenchymal progenitors is required for haematopoietic stem-cell maintenance. Nature 495, 227-230.
Grunberg, J. R., Hammarstedt, A., Hedjazifar, S., and Smith, U. (2014). The Novel Secreted Adipokine WNT1-inducible Signaling Pathway Protein 2 (WISP2) Is a Mesenchymal Cell Activator of Canonical WNT. The Journal of biological chemistry 289, 6899-6907.
Hammarstedt, A., Hedjazifar, S., Jenndahl, L., Gogg, S., Grunberg, J., Gustafson, B., Klimcakova, E., Stich, V., Langin, D., Laakso, M., et al. (2013). WISP2 regulates preadipocyte commitment and PPARgamma activation by BMP4. Proceedings of the National Academy of Sciences of the United States of America 110, 2563-2568.
Hanoun, M., Zhang, D., Mizoguchi, T., Pinho, S., Pierce, H., Kunisaki, Y., Lacombe, J., Armstrong, S. A., Duhrsen, U., and Frenette, P. S. (2014). Acute myelogenous leukemia-induced sympathetic neuropathy promotes malignancy in an altered hematopoietic stem cell niche. Cell stem cell 15, 365-375.
Hawkins, E. D., Duarte, D., Akinduro, O., Khorshed, R. A., Passaro, D., Nowicka, M., Straszkowski, L., Scott, M. K., Rothery, S., Ruivo, N., et al. (2016). T-cell acute leukaemia exhibits dynamic interactions with bone marrow microenvironments. Nature 538, 518-522.
Himburg, H. A., Harris, J. R., Ito, T., Daher, P., Russell, J. L., Quarmyne, M., Doan, P. L., Helms, K., Nakamura, M., Fixsen, E., et al. (2012). Pleiotrophin regulates the retention and self-renewal of hematopoietic stem cells in the bone marrow vascular niche. Cell reports 2, 964-975.
Hooper, A. T., Butler, J. M., Nolan, D. J., Kranz, A., lida, K., Kobayashi, M., Kopp, H. G., Shido, K., Petit, I., Yanger, K., et al. (2009). Engraftment and reconstitution of hematopoiesis is dependent on VEGFR2-mediated regeneration of sinusoidal endothelial cells. Cell stem cell 4, 263-274.
Huang, W., Yang, S., Shao, J., and Li, Y. P. (2007). Signaling and transcriptional regulation in osteoblast commitment and differentiation. Frontiers in bioscience: a journal and virtual library 12, 3068-3092.
Inada, M., Yasui, T., Nomura, S., Miyake, S., Deguchi, K., Himeno, M., Sato, M., Yamagiwa, H., Kimura, T., Yasui, N., et al. (1999). Maturational disturbance of chondrocytes in Cbfal-deficient mice. Developmental dynamics: an official publication of the American Association of Anatomists 214, 279-290.
Itkin, T., Gur-Cohen, S., Spencer, J. A., Schajnovitz, A., Ramasamy, S. K., Kusumbe, A. P., Ledergor, G., Jung, Y., Milo, I., Poulos, M. G., et al. (2016). Distinct bone marrow blood vessels differentially regulate haematopoiesis. Nature 532, 323-328.
Jacomy, M., Venturini, T., Heymann, S., and Bastian, M. (2014). ForceAtlas2, a continuous graph layout algorithm for handy network visualization designed for the Gephi software. PloS one 9, e98679.
Kalluri, R. (2016). The biology and function of fibroblasts in cancer. Nature reviews Cancer 16, 582-598.
Kanatani, M., Sugimoto, T., Nishiyama, K., and Chihara, K. (2000). Stimulatory effect of insulin-like growth factor binding protein-5 on mouse osteoclast formation and osteoclastic bone-resorbing activity. Journal of bone and mineral research: the official journal of the American Society for Bone and Mineral Research 15, 902-910.
Kfoury, Y., and Scadden, D. T. (2015). Mesenchymal cell contributions to the stem cell niche. Cell stem cell 16, 239-253.
Kiel, M. J., Yilmaz, O. H., Iwashita, T., Yilmaz, O. H., Terhorst, C., and Morrison, S. J. (2005). SLAM family receptors distinguish hematopoietic stem and progenitor cells and reveal endothelial niches for stem cells. Cell 121, 1109-1121.
Kim, Y., Lin, Q., Glazer, P. M., and Yun, Z. (2009). Hypoxic tumor microenvironment and cancer cell differentiation. Current molecular medicine 9, 425-434.
Kobayashi, H., Butler, J. M., O'Donnell, R., Kobayashi, M., Ding, B. S., Bonner, B., Chiu, V. K., Nolan, D. J., Shido, K., Benjamin, L., et al. (2010). Angiocrine factors from Akt-activated endothelial cells balance self-renewal and differentiation of haematopoietic stem cells. Nature cell biology 12, 1046-1056.
Kode, A., Manavalan, J. S., Mosialou, I., Bhagat, G., Rathinam, C. V., Luo, N., Khiabanian, H., Lee, A., Murty, V. V., Friedman, R., et al. (2014). Leukaemogenesis induced by an activating beta-catenin mutation in osteoblasts. Nature 506, 240-244.
Kowalczyk, M. S., Tirosh, I., Heckl, D., Rao, T. N., Dixit, A., Haas, B. J., Schneider, R. K., Wagers, A. J., Ebert, B. L., and Regev, A. (2015). Single-cell RNA-seq reveals changes in cell cycle and differentiation programs upon aging of hematopoietic stem cells. Genome research 25, 1860-1872.
Krevvata, M., Silva, B. C., Manavalan, J. S., Galan-Diez, M., Kode, A., Matthews, B. G., Park, D., Zhang, C. A., Galili, N., Nickolas, T. L., et al. (2014). Inhibition of leukemia cell engraftment and disease progression in mice by osteoblasts. Blood 124, 2834-2846.
Kumar, B., Garcia, M., Weng, L., Jung, X., Murakami, J. L., Hu, X., McDonald, T., Lin, A., Kumar, A. R., DiGiusto, D. L., et al. (2018). Acute myeloid leukemia transforms the bone marrow niche into a leukemia-permissive microenvironment through exosome secretion. Leukemia 32, 575-587.
Kunisaki, Y., Bruns, I., Scheiermann, C., Ahmed, J., Pinho, S., Zhang, D., Mizoguchi, T., Wei, Q., Lucas, D., Ito, K., et al. (2013). Arteriolar niches maintain haematopoietic stem cell quiescence. Nature 502, 637-643.
Kusumbe, A. P., Ramasamy, S. K., Itkin, T., Mae, M. A., Langen, U. H., Betsholtz, C., Lapidot, T., and Adams, R. H. (2016). Age-dependent modulation of vascular niches for haematopoietic stem cells. Nature 532, 380-384.
Lee, N. K., Sowa, H., Hinoi, E., Ferron, M., Ahn, J. D., Confavreux, C., Dacquin, R., Mee, P. J., McKee, M. D., Jung, D. Y., et al. (2007). Endocrine regulation of energy metabolism by the skeleton. Cell 130, 456-469.
Lo Celso, C., Fleming, H. E., Wu, J. W., Zhao, C. X., Miake-Lye, S., Fujisaki, J., Cote, D., Rowe, D. W., Lin, C. P., and Scadden, D. T. (2009). Live-animal tracking of individual haematopoietic stem/progenitor cells in their niche. Nature 457, 92-96.
Luo, G., Ducy, P., McKee, M. D., Pinero, G. J., Loyer, E., Behringer, R. R., and Karsenty, G. (1997). Spontaneous calcification of arteries and cartilage in mice lacking matrix GLA protein. Nature 386, 78-81.
Maaten, L. v. d., and Hinton, G. (2008). Visualizing data using t-SNE. Journal of machine learning research 9, 2579-2605.
Mabuchi, Y., Morikawa, S., Harada, S., Niibe, K., Suzuki, S., Renault-Mihara, F., Houlihan, D. D., Akazawa, C., Okano, H., and Matsuzaki, Y. (2013). LNGFR(+)THY-1(+)VCAM-1(hi+) cells reveal functionally distinct subpopulations in mesenchymal stem cells. Stem cell reports 1, 152-165.
Mendez-Ferrer, S., Michurina, T. V., Ferraro, F., Mazloom, A. R., Macarthur, B. D., Lira, S. A., Scadden, D. T., Ma'ayan, A., Enikolopov, G. N., and Frenette, P. S. (2010). Mesenchymal and haematopoietic stem cells form a unique bone marrow niche. Nature 466, 829-834.
Mercier, F. E., Sykes, D. B., and Scadden, D. T. (2016). Single Targeted Exon Mutation Creates a True Congenic Mouse for Competitive Hematopoietic Stem Cell Transplantation: The C57BL/6-CD45.1(STEM) Mouse. Stem cell reports 6, 985-992.
Morrison, S. J., and Scadden, D. T. (2014). The bone marrow niche for haematopoietic stem cells. Nature 505, 327-334.
Morrissy, A. S., Garzia, L., Shih, D. J., Zuyderduyn, S., Huang, X., Skowron, P., Remke, M., Cavalli, F. M., Ramaswamy, V., Lindsay, P. E., et al. (2016). Divergent clonal selection dominates medulloblastoma at recurrence. Nature.
Mossadegh-Keller, N., Sarrazin, S., Kandalla, P. K., Espinosa, L., Stanley, E. R., Nutt, S. L., Moore, J., and Sieweke, M. H. (2013). M-CSF instructs myeloid lineage fate in single haematopoietic stem cells. Nature 497, 239-243.
Muegge, K., Vila, M. P., and Durum, S. K. (1993). Interleukin-7: a cofactor for V(D)J rearrangement of the T cell receptor beta gene. Science 261, 93-95.
Muramoto, G. G., Chen, B., Cui, X., Chao, N. J., and Chute, J. P. (2006). Vascular endothelial cells produce soluble factors that mediate the recovery of human hematopoietic stem cells after radiation injury. Biology of blood and marrow transplantation: journal of the American Society for Blood and Marrow Transplantation 12, 530-540.
Nakashima, K., Zhou, X., Kunkel, G., Zhang, Z., Deng, J. M., Behringer, R. R., and de Crombrugghe, B. (2002). The novel zinc finger-containing transcription factor osterix is required for osteoblast differentiation and bone formation. Cell 108, 17-29.
Namen, A. E., Lupton, S., Hjerrild, K., Wignall, J., Mochizuki, D. Y., Schmierer, A., Mosley, B., March, C. J., Urdal, D., and Gillis, S. (1988). Stimulation of B-cell progenitors by cloned murine interleukin-7. Nature 333, 571-573.
Nilsson, S. K., Johnston, H. M., Whitty, G. A., Williams, B., Webb, R. J., Denhardt, D. T., Bertoncello, I., Bendall, L. J., Simmons, P. J., and Haylock, D. N. (2005). Osteopontin, a key component of the hematopoietic stem cell niche and regulator of primitive hematopoietic progenitor cells. Blood 106, 1232-1239.
Nishimura, R., Hata, K., Nakamura, E., Murakami, T., and Takahata, Y. (2018). Transcriptional network systems in cartilage development and disease. Histochemistry and cell biology.
Nishimura, R., Wakabayashi, M., Hata, K., Matsubara, T., Honma, S., Wakisaka, S., Kiyonari, H., Shioi, G., Yamaguchi, A., Tsumaki, N., et al. (2012). Osterix regulates calcification and degradation of chondrogenic matrices through matrix metalloproteinase 13 (MMP13) expression in association with transcription factor Runx2 during endochondral ossification. The Journal of biological chemistry 287, 33179-33190.
Omatsu, Y., Sugiyama, T., Kohara, H., Kondoh, G., Fujii, N., Kohno, K., and Nagasawa, T. (2010). The essential functions of adipo-osteogenic progenitors as the hematopoietic stem and progenitor cell niche. Immunity 33, 387-399.
Papayannopoulou, T., Craddock, C., Nakamoto, B., Priestley, G. V., and Wolf, N. S. (1995). The VLA4/VCAM-1 adhesion pathway defines contrasting mechanisms of lodgement of transplanted murine hemopoietic progenitors between bone marrow and spleen. Proceedings of the National Academy of Sciences of the United States of America 92, 9647-9651.
Passaro, D., Di Tullio, A., Abarrategi, A., Rouault-Pierre, K., Foster, K., Ariza-McNaughton, L., Montaner, B., Chakravarty, P., Bhaw, L., Diana, G., et al. (2017). Increased Vascular Permeability in the Bone Marrow Microenvironment Contributes to Disease Progression and Drug Response in Acute Myeloid Leukemia. Cancer cell 32, 324-341 e326.
Pittenger, M. F., Mackay, A. M., Beck, S. C., Jaiswal, R. K., Douglas, R., Mosca, J. D., Moorman, M. A., Simonetti, D. W., Craig, S., and Marshak, D. R. (1999). Multilineage potential of adult human mesenchymal stem cells. Science 284, 143-147.
Raaijmakers, M. H., Mukherjee, S., Guo, S., Zhang, S., Kobayashi, T., Schoonmaker, J. A., Ebert, B. L., Al-Shahrour, F., Hasserjian, R. P., Scadden, E. O., et al. (2010). Bone progenitor dysfunction induces myelodysplasia and secondary leukaemia. Nature 464, 852-857.
Rafii, S., Butler, J. M., and Ding, B. S. (2016). Angiocrine functions of organ-specific endothelial cells. Nature 529, 316-325.
Rauner, M., Franke, K., Murray, M., Singh, R. P., Hiram-Bab, S., Platzbecker, U., Gassmann, M., Socolovsky, M., Neumann, D., Gabet, Y., et al. (2016). Increased EPO Levels Are Associated With Bone Loss in Mice Lacking PHD2 in EPO-Producing Cells. Journal of bone and mineral research: the official journal of the American Society for Bone and Mineral Research 31, 1877-1887.
Satija, R., Farrell, J. A., Gennert, D., Schier, A. F., and Regev, A. (2015). Spatial reconstruction of single-cell gene expression data. Nature biotechnology 33, 495-502.
Scadden, D. T. (2014). Nice neighborhood: emerging concepts of the stem cell niche. Cell 157, 41-50.
Schepers, K., Pietras, E. M., Reynaud, D., Flach, J., Binnewies, M., Garg, T., Wagers, A. J., Hsiao, E. C., and Passegue, E. (2013). Myeloproliferative neoplasia remodels the endosteal bone marrow niche into a self-reinforcing leukemic niche. Cell stem cell 13, 285-299.
Schiebinger, G., Shu, J., Tabaka, M., Cleary, B., Subramanian, V., Solomon, A., Liu, S., Lin, S., Berube, P., and Lee, L. (2017). Reconstruction of developmental landscapes by optimal-transport analysis of single-cell gene expression sheds light on cellular reprogramming. BioRxiv, 191056.
Schmidt, T., Kharabi Masouleh, B., Loges, S., Cauwenberghs, S., Fraisl, P., Maes, C., Jonckx, B., De Keersmaecker, K., Kleppe, M., Tjwa, M., et al. (2011). Loss or inhibition of stromal-derived PlGF prolongs survival of mice with imatinib-resistant Bcr-Abl1(+) leukemia. Cancer cell 19, 740-753.
Schofield, R. (1978). The relationship between the spleen colony-forming cell and the haemopoietic stem cell. Blood cells 4, 7-25.
Silha, J. V., Mishra, S., Rosen, C. J., Beamer, W. G., Turner, R. T., Powell, D. R., and Murphy, L. J. (2003). Perturbations in bone formation and resorption in insulin-like growth factor binding protein-3 transgenic mice. Journal of bone and mineral research: the official journal of the American Society for Bone and Mineral Research 18, 1834-1841.
Soundararajan, M., and Kannan, S. (2018). Fibroblasts and mesenchymal stem cells: Two sides of the same coin? J Cell Physiol 233, 9099-9109.
Stier, S., Ko, Y., Forkert, R., Lutz, C., Neuhaus, T., Grunewald, E., Cheng, T., Dombkowski, D., Calvi, L. M., Rittling, S. R., et al. (2005). Osteopontin is a hematopoietic stem cell niche component that negatively regulates stem cell pool size. The Journal of experimental medicine 201, 1781-1791.
Strutz, F., Okada, H., Lo, C. W., Danoff, T., Carone, R. L., Tomaszewski, J. E., and Neilson, E. G. (1995). Identification and characterization of a fibroblast marker: FSP1. The Journal of cell biology 130, 393-405.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proceedings of the National Academy of Sciences of the United States of America 102, 15545-15550.
Sugimoto, Y., Takimoto, A., Akiyama, H., Kist, R., Scherer, G., Nakamura, T., Hiraki, Y., and Shukunami, C. (2013). Scx+/Sox9+ progenitors contribute to the establishment of the junction between cartilage and tendon/ligament. Development 140, 2280-2288.
Sugiyama, T., Kohara, H., Noda, M., and Nagasawa, T. (2006). Maintenance of the hematopoietic stem cell pool by CXCL12-CXCR4 chemokine signaling in bone marrow stromal cell niches. Immunity 25, 977-988.
Tontonoz, P., Hu, E., and Spiegelman, B. M. (1994). Stimulation of adipogenesis in fibroblasts by PPAR gamma 2, a lipid-activated transcription factor. Cell 79, 1147-1156.
Vortkamp, A., Lee, K., Lanske, B., Segre, G. V., Kronenberg, H. M., and Tabin, C. J. (1996). Regulation of rate of cartilage differentiation by Indian hedgehog and PTH-related protein. Science 273, 613-622.
Wagner, T., Wirth, J., Meyer, J., Zabel, B., Held, M., Zimmer, J., Pasantes, J., Bricarelli, F. D., Keutel, J., Hustert, E., et al. (1994). Autosomal sex reversal and campomelic dysplasia are caused by mutations in and around the SRY-related gene SOX9. Cell 79, 1111-1120.
Wolf, F., Hamey, F., and Plass, M. (2017a). Graph abstraction reconciles clustering with trajectory inference through a topology preserving map of single cells. bioRxiv 2017.
Wolf, F. A., Angerer, P., and Theis, F. J. (2018). SCANPY: large-scale single-cell gene expression data analysis. Genome Biol 19, 15.
Wolf, F. A., Hamey, F., Plass, M., Solana, J., Dahlin, J. S., Gottgens, B., Rajewsky, N., Simon, L., and Theis, F. J. (2017b). Graph abstraction reconciles clustering with trajectory inference through a topology preserving map of single cells. bioRxiv.
Worthley, D. L., Churchill, M., Compton, J. T., Tailor, Y., Rao, M., Si, Y., Levin, D., Schwartz, M. G., Uygur, A., Hayakawa, Y., et al. (2015). Gremlin 1 identifies a skeletal stem cell with bone, cartilage, and reticular stromal potential. Cell 160, 269-284.
Wozney, J. M., Rosen, V., Celeste, A. J., Mitsock, L. M., Whitters, M. J., Kriz, R. W., Hewick, R. M., and Wang, E. A. (1988). Novel regulators of bone formation: molecular clones and activities. Science 242, 1528-1534.
Xu, C., Gao, X., Wei, Q., Nakahara, F., Zimmerman, S. E., Mar, J., and Frenette, P. S. (2018). Stem cell factor is selectively secreted by arterial endothelial cells in bone marrow. Nature communications 9, 2449.
Yang, L., Tsang, K. Y., Tang, H. C., Chan, D., and Cheah, K. S. (2014). Hypertrophic chondrocytes can become osteoblasts and osteocytes in endochondral bone formation. Proceedings of the National Academy of Sciences of the United States of America 111, 12097-12102.
Yoder, M. C. (2012). Human endothelial progenitor cells. Cold Spring Harbor perspectives in medicine 2, a006692.
Young, M. F., Kerr, J. M., Termine, J. D., Wewer, U. M., Wang, M. G., McBride, O. W., and Fisher, L. W. (1990). cDNA cloning, mRNA distribution and heterogeneity, chromosomal location, and RFLP analysis of human osteopontin (OPN). Genomics 7, 491-502.
Yu, V. W., Lymperi, S., Oki, T., Jones, A., Swiatek, P., Vasic, R., Ferraro, F., and Scadden, D. T. (2016). Distinctive Mesenchymal-Parenchymal Cell Pairings Govern B Cell Differentiation in the Bone Marrow. Stem cell reports 7, 220-235.
Yu, V. W., Saez, B., Cook, C., Lotinun, S., Pardo-Saganta, A., Wang, Y. H., Lymperi, S., Ferraro, F., Raaijmakers, M. H., Wu, J. Y., et al. (2015). Specific bone cells produce DLL4 to generate thymus-seeding progenitors from bone marrow. The Journal of experimental medicine 212, 759-774.
Zambetti, N. A., Ping, Z., Chen, S., Kenswil, K. J., Mylona, M. A., Sanders, M. A., Hoogenboezem, R. M., Bindels, E. M., Adisty, M. N., Van Strien, P. M., et al. (2016). Mesenchymal Inflammation Drives Genotoxic Stress in Hematopoietic Stem Cells and Predicts Disease Evolution in Human Pre-leukemia. Cell stem cell 19, 613-627.
Zhang, B., Ho, Y. W., Huang, Q., Maeda, T., Lin, A., Lee, S. U., Hair, A., Holyoake, T. L., Huettner, C., and Bhatia, R. (2012). Altered microenvironmental regulation of leukemic and normal stem cells in chronic myelogenous leukemia. Cancer cell 21, 577-592.
Zhang, J., Niu, C., Ye, L., Huang, H., He, X., Tong, W. G., Ross, J., Haug, J., Johnson, T., Feng, J. Q., et al. (2003). Identification of the haematopoietic stem cell niche and control of the niche size. Nature 425, 836-841.
Zhou, B. O., Yue, R., Murphy, M. M., Peyer, J. G., and Morrison, S. J. (2014a). Leptin-receptor-expressing mesenchymal stromal cells represent the main source of bone formed by adult bone marrow. Cell stem cell 15, 154-168.
Zhou, X., von der Mark, K., Henry, S., Norton, W., Adams, H., and de Crombrugghe, B. (2014b). Chondrocytes transdifferentiate into osteoblasts in endochondral bone during development, postnatal growth and fracture healing in mice. PLoS genetics 10, e1004820.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of remodeling a stromal cell landscape comprising:
administering a modulating agent to a subject or a cell population that induces a shift in the stromal cell landscape from a disease-associated stromal cell landscape to a homeostatic stromal cell landscape.

2. The method of claim 1, wherein the shift in stromal cells from a disease-associated stromal cell landscape to a homeostatic stromal cell landscape comprises a change in the proportion of preosteoblasts.

3. The method of claim 2, wherein the change in the proportion of preosteoblasts comprises a change in the relative proportion of OLC-1 cells to OLC-2 cells.

4. The method of claim 3, wherein the change in the relative proportion of OLC-1 cells to OLC-2 cells comprises a decrease in OLC-1 cells and an increase in OLC-2 cells.

5. The method of claim 1, wherein the shift in stromal cells from a disease-associated stromal cell landscape to a homeostatic stromal cell landscape comprises a change in the relative proportion of bone marrow derived endothelial cell subtypes.

6. The method of claim 5, wherein the change in the change in the relative proportion of bone marrow derived endothelial cell subtypes comprises an increase in sinusoidal bone marrow derived endothelial cells and a decrease in arterial bone marrow derived endothelial cells.

7. The method of claim 1, wherein the shift in stromal cells from a disease-associated stromal cell landscape to a homeostatic stromal cell landscape comprises a change in the relative proportion of chondrocyte subtypes.

8. The method of claim 7, wherein the change in the relative proportion of chondrocyte subtypes comprises a decrease in chondrocyte hypertrophic cell subtype and an increase in chondrocyte progenitor cell subtype.

9. The method of claim 1, wherein the shift in stromal cells from a disease-associated stromal cell landscape to a homeostatic stromal cell landscape comprises a change in the relative proportion of fibroblast subtypes.

10. The method of claim 9, wherein the change in the relative proportion of fibroblast subtypes comprises an increase in fibroblast subtype-3 and a decrease in fibroblast subtype-4.

11. The method of claim 1, wherein the shift in stromal cells from a disease-associated stromal cell landscape to a homeostatic stromal cell landscape comprises a change in the relative proportion in mesenchymal stem/stromal cell (MSC) subtypes.

12. The method of claim 11, wherein the change in the relative proportion in mesenchymal stem/stromal cell (MSC) sub-types comprises a decrease in MSC-2 subtype and an increase in MSC-3 and MSC-4 subtypes.

13. The method of claim 1, wherein the shift in the stromal cell landscape comprises a change in the distance in gene expression space between OLC-1, OLC-2, bone marrow derived endothelial cell subtypes, chondrocyte subtypes, fibroblast subtypes, mesenchymal stem/stromal cell (MSC) subtypes, or a combination thereof.

14. The method of claim 13, wherein the distance is measured by a Euclidean distance, Pearson coefficient, Spearman coefficient, or a combination thereof.

15. The method of claim 14, wherein the gene expression space comprises 10 or more genes, 20 or more genes, 30 or more genes, 40 or more genes, 50 or more genes, 100 or more genes, 500 or more genes, or 1000 or more genes.

16. The method of claim 15, wherein remodeling the stromal cell landscape comprises increasing or decreasing the expression of one or more genes, gene programs, gene expression cassettes, gene expression signatures, or a combination thereof.

17. The method of claim 16, wherein the change in the gene expression space is characterized by a change in the expression of one or more genes as in any one of Tables 1-8 or a combination thereof or an expression signature derived therefrom.

18. The method of claim 15, wherein identifying differences in stromal cell states in the shift in the stromal cell landscape comprises comparing a gene expression distribution of a stromal cell type or subtype in the diseased stromal cell landscape with a gene expression distribution of the stromal cell type or subtype in the homeostatic stromal cell landscape as determined by single cell RNA-sequencing (scRNA-seq).

19. The method of claim 1, wherein the shift in the stromal cell landscape from a disease-associated stromal cell landscape to a homeostatic stromal cell landscape increases committed MSCs and decreases osteoprogenitor cells.

20. The method of claim 1, wherein the subject suffers from a hematological disease.

21. The method of claim 20, wherein the hematological disease is a blood cancer.

22. The method of claim 21, wherein the blood cancer is a leukemia.

23. The method of claim 20, wherein the blood cancer is acute lymphocytic leukemia, acute myeloid leukemia, chronic lymphocytic leukemia, chronic myeloid leukemia, hairy cell leukemia, myelodysplastic syndromes, acute promyelocytic leukemia, or myeloproliferative neoplasm.

24. The method of claim 1, wherein the cell population comprises a single cell type and/or subtype, a combination of cell types and/or subtypes, a cell-based therapeutic, an explant, or an organoid.

25. The method of claim 24, wherein the cell population is a non-hematopoietic stromal cell or cell population.

26. The method of claim 24, wherein the cell or cell population is a MSC, OLC, bone marrow derived endothelial cell, chondrocyte, or a fibroblast cell or cell population.

27. The method of claim 1, wherein the modulating agent is a therapeutic antibody, antibody fragment, antibody-like protein scaffold, aptamer, polypeptide, protein, genetic modifying agent, small molecule, small molecule degrader, or combination thereof.

28. The method of claim 27, wherein the genetic modifying agent is a CRISPR-Cas system, a TALEN, a Zn-finger nuclease, or a meganuclease.

29. An isolated or engineered stromal cell or cell population prepared by a method as in any one of claims 1-28.

30. An isolated or engineered mesenchymal stem/stromal cell (MSC) or MSC cell population, wherein the MSC or MSC cell population is characterized by a gene signature comprised of one or more genes of Table 1.

31. The isolated or engineered MSC or MSC cell population of claim 30, wherein the MSC or MSC cell population is characterized by a gene signature comprised of one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4.

32. The isolated or engineered MSC or MSC cell population of claim 30, wherein the MSC or MSC cell population does not express one or more of Thy1, Ly6a (Sca-1), NG2 (Cspg4) or Nestin (Nes).

33. The isolated or engineered MSC or MSC cell population of claim 30, wherein the gene signature further comprises one or more of Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxc1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1.

34. An isolated or engineered osteolineage cell (OLC) or OLC population, wherein the isolated or engineered OLC or OLC population is characterized by a gene signature comprising one or more genes of Table 2.

35. The isolated or engineered OLC or OLC population, of claim 34, wherein the OLC or OLC population is characterized by a gene signature comprising one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1.

36. The isolated or engineered OLC or OLC population of claim 34, wherein the OLC or OLC population expresses Bglap and Spp1.

37. The isolated or engineered OLC or OLC population of claim 34, wherein the gene signature further comprises one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmem119, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbp11, Col3a1, Ptn, Col6a2, Tnn, Npy, Col6a1, Omd, Dcn, Tgfbi, Col6a3, or Acan.

38. The isolated or engineered OLC or OLC population of claim 34, wherein the gene signature further comprises one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdm1, Mia, Myoc, Nrtn, Pdzrn4, Spp1, Pth1r, Sox9, Acan, or Mmp13.

39. An isolated or engineered pericyte or pericyte population, wherein the isolated or engineered pericyte is characterized by a gene signature comprising one or more genes in Table 3.

40. The isolated or engineered pericyte or pericyte population of claim 39, wherein the gene signature further comprises one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, or Cygp.

41. The isolated or engineered pericyte or pericyte population of claim 39, wherein the gene signature further comprises one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11.

42. The isolated or engineered pericyte or pericyte population of claim 39, wherein the gene signature further comprises one or more Acta2, Myh11, Mcam, Jag1, and Il6.

43. An isolated or engineered chondrocyte or chondrocyte population, wherein the isolated or engineered chondrocyte population is characterized by a gene signature comprising one or more genes in Table 4.

44. The isolated or engineered chondrocyte or chondrocyte population of claim 43, wherein the gene signature further comprises one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst11, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, I17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a.

45. The isolated or engineered chondrocyte or chondrocyte population of claim 43, wherein the gene signature further comprises one or more of Sox9, Col11a2, Acan, or Col2a1.

46. The isolated or engineered chondrocyte or chondrocyte population of claim 43, wherein the gene signature further comprises one or more of Runx2, Ihh, Mef2c, or Col10a1.

47. The isolated or engineered chondrocyte or chondrocyte population of claim 43, wherein the gene signature further comprises one or more of Grem1, Runx2, Sp7, Alp1, or Spp1.

48. The isolated or engineered chondrocyte or chondrocyte population of claim 43, wherein the chondrocyte expresses one or more of Ihh, Pth1r, Mef2c, Col10a1, Ibsp, Mmp13, or Grem 1.

49. The isolated or engineered chondrocyte or chondrocyte population of claim 43, wherein the gene signature further comprises one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst1, Epyc, Serpinh1, Gnb211, Fscn1, Pla2g5, Rcn1, Sox9, Bglap, Sp7, Fn1, Ube2s, Hmgb1, Ckap4, Clec11a, I17b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

50. An isolated or engineered fibroblast or fibroblast population, wherein the isolated or engineered fibroblast or fibroblast population is characterized by a gene signature comprising one or more genes of Table 5.

51. The isolated or engineered fibroblast or fibroblast population of claim 50, wherein the gene signature further comprises one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc.

52. The isolated or engineered fibroblast or fibroblast population of claim 50, wherein the gene signature comprises one or more of Fibronectin-1 (Fn1), Fibroblast Specific Protein-1 (S100a4), Col1a1, Col1a2, Lum, Col22a1, or Twist2.

53. The isolated or engineered fibroblast or fibroblast population of claim 50, wherein the gene signature comprises one or more of Sox9, Acan, and Col2a1.

54. The isolated or engineered fibroblast or fibroblast population of claim 50, wherein the gene signature comprises one or more of Cd34, Ly6a, Pdgfra, Thy1 and Cd44, and not Cdh5, or Acta2.

55. The isolated or engineered fibroblast or fibroblast population of claim 50, wherein the gene signature comprises one or more of Sox-9, Scleraxis (Scx), Spp1, Cspg4, CD73 (Nt5e), or Cartilage Intermediate Layer Protein (Cilp).

56. The isolated or engineered fibroblast or fibroblast population of claim 50, wherein the gene signature further comprises one or more of S1004a, Dcn, Sema3c, or Cxcl12.

57. An isolated or engineered bone marrow derived endothelial cell (BMEC) or BMEC population, wherein the isolated or engineered fibroblast or fibroblast population is characterized by a gene signature comprising one or more genes of Table 6.

58. The isolated or engineered BMEC or BMEC population of claim 57, wherein the gene signature comprises one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x.

59. The isolated or engineered BMEC or BMEC population of claim 57, wherein the gene signature comprises one or more of Flt4 (Vegfr-3) and Ly6a (Sca-1), wherein Ly6a expression, when present in the gene signature, is reduced as compared to a suitable control.

60. The isolated or engineered BMEC or BMEC population of claim 57, wherein the gene signature comprises one or more of Pecam1, Cdh5, Cd34, Tek, Lepr, Cxcl12, or Kitl.

61. The isolated or engineered BMEC or BMEC population of claim 57, wherein the gene signature comprises one or more of Flt4, Ly6a, Icam1, or Sele.

62. The isolated or engineered BMEC or BMEC population of claim 57, wherein the gene signature comprises one or more of Mafb, Cebpb, Xbp1, Nr2f2, Irf8, Ybx1, Ebf1, Sox17, Mxd4, Id1, Meox2, Tshz2, Tcf15, Meox1, Tfpi, I116stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

63. A method of treating a hematological disease comprising:
administering to a subject in need thereof the isolated or engineered cell or cell population as in any one of claims 29-62.

64. A method of screening for one or more agents capable of modulating a stromal cell state, comprising:
contacting a stromal cell population having an initial cell state with a test modulating agent or library of modulating agents, wherein the stromal cell population optionally contains leukemia cells;
determining one or more fractions of stromal cell states including one or more fraction(s) of a mesenchymal stem/stromal cell (MSC), an OLC, a chondrocyte, a fibroblast, a pericyte, a bone marrow derived endothelial cell (BMEC), or a combination thereof; and
selecting modulating agents that shifts the initial stromal cell state to a desired stromal cell state, wherein the desired stromal cell fraction in the stromal cell population is above a set cutoff limit.

65. The method of claim 64, wherein determining one or more fractions of stromal cell states further comprises determining one or more MSC subtype, one or more OLC types, one or more chondrocyte types, one or more fibroblast types, one or more BMEC types, one or more pericyte subtype, or a combination thereof.

66. The method of claim 64 or 65, wherein the stromal cell population is obtained from a subject to be treated.

67. The method of claim 64 or 65, wherein determining one or more fractions of stromal cell states comprises identifying a MSC gene signature, an OLC gene signature, a chondrocyte gene signature, a fibroblast gene signature, a BMEC gene signature, a pericyte gene signature.

68. The method of claim 67, wherein the MSC gene signature comprises:
a. one or more genes of Table 1;
b. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or
c. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxc1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1,
and wherein the MCS optionally does not express one or more of Thy1, Ly6a (Sca-1), NG2 (Cspg4) or Nestin (Nes).

69. The method of claim 67, wherein the OLC gene signature comprises:
a. one or more genes of Table 2;
b. one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;
c. one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmeml19, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbp11, Col3a1, Ptn, Col6a2, Tnn, Npy, Col6a1, Omd, Dcn, Tgfbi, Col6a3, or Acan; or
d. one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13,
and wherein the OLC optionally expresses Bglap and Spp1.

70. The method of claim 67, wherein the chondrocyte gene signature comprises:
a. one or more genes of Table 4;
b. one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst1, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, I17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;
c. one or more of Sox9, Col11a2, Acan, or Col2a1;
d. one or more of Runx2, Ihh, Mef2c, or Col10a1;
e. one or more of Grem, Runx2, Sp7, Alp1, or Spp1;
f. one or more of Ihh, Pth1r, Mef2c, Col10a1, Ibsp, Mmp13, Grem 1; or
g. one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, Il17b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

71. The method of claim 67, wherein the fibroblast gene signature comprises:
a. one or more genes of Table 5;
b. one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;
c. Fibronectin-1 (Fn1), Fibroblast Specific Protein-1 (S100a4), Col1a1, Col1a2, Lum, Col22a1, or Twist2;
d. one or more of Sox9, Acan, and Col2a1;
e. Cd34, Ly6a, Pdgfra, Thy1 and Cd44, and not Cdh5, or Acta2;
f. one or more of Sox-9, Scleraxis (Scx), Spp1, Cspg4, CD73 (Nt5e), and Cartilage Intermediate Layer Protein (Cilp); or
g. one or more of S1004a, Dcn, Sema3c, or Cxcl12.

72. The method of claim 67, wherein the BMEC gene signature comprises:
a. one or more genes of Table 6;
b. one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x;
c. one or more of Flt4 (Vegfr-3) or Ly6a (Sca-1);
d. one or more of Pecam1, Cdh5, Cd34, Tek, Lepr, Cxcl12, or Kitl;
e. one or more of Flt4, Ly6a, Icam1, or Sele;
f. one or more of Mafb, Cebpb, Xbp1, Nr2f2, Irf8, Ybx1, Ebf1, Sox17, Mxd4, Id1, Meox2, Tshz2, Tcf15, Meox1, Tfpi, I1l6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, lfitm2; or
g. one or more of Mafb, Cebpb, Xbp1, Nr2f2, Irf8, Ybx1, Ebf1, Sox17, Mxd4, Id1, Meox2, Tshz2, Tcf15, Meox1, Tfpi, I1l6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

73. The method of claim 67, wherein the pericyte gene signature comprises:
a. one or more genes in Table 3;
b. one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, Cygp;
c. one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11; or
d. one or more of Acta2, Myh11, Mcam, Jag1, or Il6.

74. The method of claim 64, wherein the modulating agent that shifts the initial stromal cell state to the desired stromal cell state is capable of remodeling in a hematological disease.

75. A method of screening for one or more agents capable of modulating osteogenic and/or adipogenic differentiation in a hematological disease comprising:
contacting a cell population with a test modulating agent, wherein the cell population comprises MSC(s), OLC(s), and leukemia cells; and
selecting modulating agents that change the regulation of one or more of Grem1, Bmp4, Sp7, Runx2, Bglap1, Bglap2, Bglap3, Adipoq, Wisp2, Mgp, Igbfp5, Igbfp3, Mmp2, Mmp11, or Mmp13.

76. A method of screening for one or more agents capable of remodeling in a hematological disease comprising:
contacting a cell population with a test modulating agent, wherein the cell population comprises MSC(s), OLC(s), and leukemia cells; and
selecting one or more modulating agents that
a. change the proportion of prerosteoblasts in the cell population;
b. change the relative proportion of OLC-1 to OLC-2 in the cell population;
c. change the relative proportion of hypertrophic chondrocytes to progenitor chondrocytes in the cell population;
d. change the relative proportion of subtype-3 (Cluster 16) fibroblasts to subtype-4 fibroblasts (Cluster 3); or
e. a combination thereof.

77. A method of detecting a mesenchymal stem/stromal cell (MSC) from a population of stromal cells comprising:
detecting in a sample the expression or activity of a MSC gene expression signature,
wherein detection of the MSC gene expression signature indicates MSCs in the sample, and
wherein the MSC gene expression signature comprises:
a. one or more genes of Table 1;
b. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or
c. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxc1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1;
and wherein the MCS optionally does not express one or more of Thy1, Ly6a (Sca-1), NG2 (Cspg4) or Nestin (Nes).

78. A method of detecting an osteolineage cell (OLC) from a population of stromal cells comprising:
detecting in a sample the expression or activity of an OLC gene expression signature,
wherein detection of the OLC gene expression signature indicates OLCs in the sample, and
wherein the OLC gene expression signature comprises
a. one or more genes of Table 2;
b. one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;
c. one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmeml19, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbp11, Col3a1, Ptn, Col6a2, Tnn, Npy, Col6a1, Omd, Dcn, Tgfbi, Col6a3, or Acan; or
d. one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13;
and wherein the OLC optionally expresses Bglap and Spp1.

79. A method of detecting a chondrocyte from a population of stromal cells comprising:
detecting in a sample the expression or activity of a chondrocyte gene expression signature,
wherein detection of the chondrocyte gene expression signature indicates chondrocytes in the sample, and
wherein the chondrocyte gene expression signature comprises
a. one or more genes of Table 4;
b. one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst1, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, I17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;
c. one or more of Sox9, Col11a2, Acan, or Col2a1;
d. one or more of Runx2, Ihh, Mef2c, or Col10a1;
e. one or more of Grem, Runx2, Sp7, Alp1, or Spp1;
f. one or more of Ihh, Pth1r, Mef2c, Col10a1, Ibsp, Mmp13, or Grem 1; or
g. one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, Il17b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

80. A method of detecting a fibroblast from a population of stromal cells comprising:
detecting in a sample the expression or activity of a fibroblast gene expression signature,
wherein detection of the fibroblast gene expression signature indicates fibroblasts in the sample, and
wherein the fibroblast gene expression signature comprises
a. one or more genes of Table 5;
b. one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;
c. Fibronectin-1 (Fn1), Fibroblast Specific Protein-1 (S100a4), Col1a1, Col1a2, Lum, Col22a1, or Twist2;
d. one or more of Sox9, Acan, and Col2a1;
e. Cd34, Ly6a, Pdgfra, Thy1 and Cd44, and not Cdh5, or Acta2;
f. one or more of Sox-9, Scleraxis (Scx), Spp1, Cspg4, CD73 (Nt5e), and Cartilage Intermediate Layer Protein (Cilp); or
g. one or more of S1004a, Dcn, Sema3c, or Cxcl12.

81. A method of detecting a bone marrow derived endothelial cell (BMEC) from a population of stromal cells comprising:
detecting in a sample the expression or activity of a BMEC gene expression signature,
wherein detection of the BMEC gene expression signature indicates BMECs in the sample, and
wherein the fibroblast gene expression signature comprises
a. one or more genes of Table 6;
b. one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x;
c. one or more of Flt4 (Vegfr-3) or Ly6a (Sca-1);
d. one or more of Pecam1, Cdh5, Cd34, Tek, Lepr, Cxcl12, or Kitl;
e. one or more of Flt4, Ly6a, Icam1, or Sele;
f. one or more of Mafb, Cebpb, Xbp1, Nr2f2, Irf8, Ybx1, Ebf1, Sox17, Mxd4, Id1, Meox2, Tshz2, Tcf15, Meox1, Tfpi, I1l6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, lfitm2; or
g. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

82. A method of detecting a pericyte from a population of stromal cells comprising:
detecting in a sample the expression or activity of a pericyte gene expression signature,
wherein detection of the pericyte gene expression signature indicates pericyte s in the sample, and
wherein the fibroblast gene expression signature comprises
a. one or more genes in Table 3;
b. one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, or Cygp;
c. one or more of Cspg4, Ngfr, Des, Myh11, Acta2, Rgs5, Thy1, Pdgtfrb, Nes, Lepr, Cdh2, Cxcl12, Kitl, Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11; or
d. one or more of Acta2, Myh11, Mcam, Jag1, or Il6.

83. The method of any one of claims 77-82, wherein the sample is obtained from the blood or bone marrow.

84. A method of preparing a mesenchymal stem/stromal cell (MSC) enriched cell population a stromal cell population comprising:
enriching the population of stromal cells for cells that have an MSC gene signature, wherein the gene signature comprises
a. one or more genes of Table 1;
b. one or more of Cebpa, Zeb2, Runx2, Ebf1, Foxc1, Cebpb, Ar, Fos, Id4, Klf6, Irf1, Runx2, Jun, Snaj2, Maf, Zthx4, Id3, Egr1, Junb, Hp, Lpl, Gdpd2, Serping, Dpep1, Grem1, Pappa, Chrdl1, Fbln5, Vcam1, Kng1, H2-Q10, Cdh11, Mme, Tmem176b, Csf1, H2-K1, Serpine2, H2-D1, Tnc, Cdh2, Pdgtra, Esm1, Gas6, Cxcl14, Sfrp4, Wisp2, Agt, Il34, Fst, Fgf7, Il1rn, C2, Igfpb4, Serpina1, Cbln1, Apoe, Ibsp, Igfbp5, Gpx3, Pdzrn4, Rarres2, Vegfa, 1500009L16Rik, Serpina3g, Cyp1b1, Ebt3, Arrdc4, Kng2, Slc26a7, Marc1, Ms4ad4, Wdr86, Serpina3c, Tmem176a, Cldn10, Trt, Gpr88, Nnmt, Gm4951, Cd1d1, Plpp3, or Ackr4; or
c. Nte5, Vcam1, Eng, Thy1, Ly6a, Grem1, Cspg4, Nes, Runx2, Col1A1, Erg1, Junb, Fosb, Cebpb, Klf6, Nr4a1, Klf2, Atf3, Klf4, Maff, Nfia, Smad6, Hey1, Sp7, Id1, Ifrd1, Trib1, Rrad, Odc1, Actb, Notch2, AlpI, Mmp13, Raph1, Tnfsf11, Cxc1, Adamts1, Cc17, Serpine1, Cc12, Apod, Cbln1, Pam, Col8a1, Wif1, Olfml3, Gdf10, Cyr61, Nog, Angpt4, Metrn1, Trabd2b, Adamts5, Igfbp4, Cxcl12, Igfbp5, Lepr, Cxcl12, Kit1, Grem1, or Angpt1,
and wherein the MCS optionally does not express one or more of Thy1, Ly6a (Sca-1), NG2 (Cspg4) or Nestin (Nes).

85. A method of preparing an osteolineage (OLC) enriched cell population a stromal cell population comprising:
enriching the population of stromal cells for cells that have an OLC gene signature, wherein the gene signature comprises
a. one or more genes of Table 2;
b. one or more of Vdr, Satb2, Sp7, Runx2, Tbx2, Zeb2, Dlx5, Dlx6, Zfhx4, Hey1, Irx5, Id3, Mxd4, Mef2c, Esr1, Maf, Smad6, Sox4, Cebpb, Meis3, Mmp13, Tnc, Cfh, Alp1, Lrp4, Cdh11, Casm1, Cdh2, Slit2, Bmp3, Cdh15, Fat3, Pard6g, Litr, Cp, Ptprd, Olfml3 Fign, Cd63, Fap, Dmp1, Angpt4, Chn1, Ibsp, Wisp1, Wif1, Metrn1, Vldlr, Podnl1, Col22a1, Ndnf, Mmp14, Pgf, Lox11, Mfap2, Srpx2, Agt, Tmem59, Vstm4, Col8a1, Cxcl12, Bglap2, Car3, Kcnk2, Slc36a2, Ifitm5, Hpgd, Limch1, Gm44029, Hvcn1, Tnfrsf19, Col13a1, Fam78b, Gja1, Cnn2, Ppfibp2, Cldn10, Dapk2, Tmp1, Bglap3, or Ramp1;
c. one or more of Runx2, Sp7, Grem1, Lepr, Cxcl12, Kit1, Bglap, Cd200, Spp1, Sox9, Id4, Ebf1, Ebf3, Cebpa, Foxc1, Snai2, Maf, Runx1, Thra, Plagl1, Mafb, Vdr, Cebpb, Tcf712, Bhlhe40, Snai1, Creb311, Zbtb7c, Gm22, Tcf7, Nr4a2, Atf3, Prrx2, Fbln5, H2-K1, H2-D1, Hp, Fstl1, Tmem176b, B2m, Pappa, Dpep1, Islr, Vcam1, Lepr, Mmp13, Cd200, Itgb5, Lifr, Postn, Slit2, Timp1, Lrp4, Tspan6, Ctsc, Cpz, Prss35, Tmeml19, Lox, Cryab, Pdzd2, Fyn, Gucala, Rerg, Sema4d, Vcam, Aspn, Slc20a2, Plat, Fmod, Fn1, Aebop1, Angpt12, Prkcdbp, Prelp, Cxcl12, Igfbp4, Cxcl14, Gas6, Apoe, Igfbp7, Col8a1, Serping1, Igfbp5, Igf1, Kit1, Spp1, Serpine2, Fam20c, Bmp8a, Dmp1, Ibsp, Pros1, Srpx2, Mgll, Timp3, Col11a2, Cgref1, Col1a1, Cthrc1, Sparc, Col22a1, Col5a2, Fkbp11, Col3a1, Ptn, Col6a2, Tnn, Npy, Col6a1, Omd, Dcn, Tgfbi, Col6a3, or Acan; or d. one or more of Runx2, Sp7, Grem1, Bglap, Cxcl12, Kit1, Osr1, Foxd1, Sox5, Osr2, Erg, Nfatc2, Mef2c, Sp7, Zbtb7c, Runx2, Snai2, Zfhx4, Dlx6, Meox1, Prrx1, Scx, Hic1, Peg3, Etv5, Ltbp1, Tspan8, Emb, Slc16a2, Tspan13, Creb5, Scara3, Prg4, Clu, plxdc1, Cdon, Fbln7, Ntn1, Nt5e, Thbd, Pth1r, Alp1, Cadm1, Cd200, Susd5, Rarres1, Ptprz1, Plat, Tnfrsf11b, Lpar3, Cspg4, Postn, S1pr1, Enah, Aspn, Cald1, Wnt5b, Adam12, Tnc, Pak1, Lpl, Mfap4, Cntfr, Fbln2, Fgl2, Gpc3, Ogn, Slc1a3, Spock2, Fbln5, Rgp1, Smoc1, C5ar1, Fzd9, Npr2, Fzd10, Cxcl14, Wif1, Arsi, Col12a1, Mgp, Itgbl1, Igf1, Smoc2, Spon2, Fst, Sbsn, Gas1, Sod3, Mmp3, Cilp, Pla2g2e, Fam213a, Acp5, Col15a1, Bglap2, Bglap3, Ibsp, Thbs4, Frzb, Bmp8a, Dkk1, Scube1, Chad, Spp1, Col11a2, Ptn, Ostn, Tnn, Mmp14, Gpx3, Cthrc1, Cxcl12, Prss12, Rbln1, Penk, Col8a1, Vipr2, Apod, Cpxm2, Rarres2, C4b, Sparcl1, Ly6e, R3hdml, Mia, Myoc, Nrtn, Pdzrn4, Spp1, Pth1r, Sox9, Acan, or Mmp13;
and wherein the OLC optionally expresses Bglap and Spp1.

86. A method of preparing a chondrocyte enriched cell population a stromal cell population comprising:
enriching the population of stromal cells for cells that have a chondrocyte gene signature, wherein the gene signature comprises
a. one or more genes of Table 4;
b. one or more of Barx1, Pitx1, Foxd1, Osr2, Tbx18, Runx3, Osr2, Tbx18, Runx3, Peg3, Bhlhe41, Batf3, Plagl1, Sp7, Sox8, Lef1, Shox2, Zbtb20, Foxa3, Mef2c, Egr2, Pax1, Runx2, Prg4, Cpe, Mfi2, Scara3, Cpm, Chst11, Unc5q, Col11a1, Slc2a5, Slc26a2, Cspg4, Prc1, Fgfr3, Nid2, Spon1, Slc40a, Efemp1, Susd5, Fxyd3, Alp1, Corin, Tpd5211, Sema3d, F5, Slc38a3, Cytl1, Rbp4, Vit, Clip, Fam19a5, Col9a3, Col9a1, Col9a2, Matn3, Hapln1, Sfrp5, Notum, Mia, lhh, Mgst2, Rarres1, Gpld1, I17b, Bglap, 1500015010Rik, Itm2a, Crispld1, Meg3, Cenpp, Fxyd2, 3110079O15Rik, Lect1, Papss2, SAyt8, Stmn1, Lockd, Chil1, Calml3, Ncmap, Serpina1d, Serpina 1b, Serpina 1c, Sic6a1, or Serpina1a;
c. one or more of Sox9, Col11a2, Acan, or Col2a1;
d. one or more of Runx2, Ihh, Mef2c, or Col10a1;
e. one or more of Grem, Runx2, Sp7, Alp1, or Spp1;
f. one or more of Ihh, Pth1r, Mef2c, Col10a1, Ibsp, Mmp13, Grem 1; or
g. one or more of Prg4, Gas1, Clu, Dcn, Cilp, Scara3, Cytl1, Igfbp7, Cilp2, Cpe, Sod3, Cd81, Abi3 bp, Creb5, Gsn, Crip2, Vit, Fhl1, Pam, Cd9, Prrx1, Vim, Col11a2, Col9a1, Col2a1, Col9a2, Col27a1, Col9a3, Hapln1, Acan, Matn3, Col11a1, Pth1r, Mia, Pcolce2, Chst11, Epyc, Serpinh1, Gnb211, Fscn1, Pla2g5, Rcn1, Sox9, Bglap, Sp7, Fn1, Ube2s, Hmgb1, Ckap4, Clec11a, Il17b, Ybx1, Tmem97, Rbm3, Slc26a2, C1qtnf3, Fkbp2, Prelp, Apoe, Cst3, Spon1, Olfml3, Wif1, Lef1, Notum, Emb, Col1a2, Sfrp5, Omd, Ctsd, Zbtb20, Islr, B2m, Ly6e, Alp1, Spp1, Chad, Timp3, Mef2c, Sparc, Ihh, Junb, Txnip, Rarres1, Scrg1, Sema3d, Colgalt2, Serinc5, Slc38a2, Ddit41, Egr1, Runx2, or Cxcl12.

87. A method of preparing a fibroblast enriched cell population a stromal cell population comprising:
enriching the population of stromal cells for cells that have a fibroblast gene signature, wherein the gene signature comprises
a. one or more genes of Table 5;
b. one or more of Scx, Barx1, Trps1, Hoxd9, Pitx1, Prrx1, Rora, Prrx2, Meox2, Ebf2, Osr2, Ebf1, Dlx3, Zfhx2, Meox1, Etv4, Mkx, Dcn, Clu, Abi3 bp, Prelp, Lox, Tnxb, Col3a1, Vcan, Vi, Mfap5, Col14a1, Aspn, Pdpn, Pdgfra, F13a1, Clic5, Gpr1, Emilin2, Has1, Mtap4, Gas2, Ntng1, Serpinf1, Postn, Angpt17, Clip2, Clip, Sod3, Slurp1, Spp1, Clec3b, Igfbp6, Thds4, Dpt, Gsn, Fndc1, Pla1a, Adamts15, Figf, Htra4, Rspo2, Mstn, Ptx4, Spock3, Cpxm2, Itgbl1, Anxa8, Fxyd5, Fxyd6, Egln3, Ptgis, I133, Fgf9, Tppp3, Crlp1, Mustn1, Celf2, Tmod2, Ly6a, Fez1, Lysmd2, Pcsk6, 2210407C18Rik, Aldh1a3, Rtn1, Rab37, Lnmd, Chod1, Fam159b, Prph, or Insc;
c. Fibronectin-1 (Fn1), Fibroblast Specific Protein-1 (S100a4), Col1a1, Col1a2, Lum, Col22a1, or Twist2;
d. one or more of Sox9, Acan, and Col2a1;
e. Cd34, Ly6a, Pdgfra, Thy1 and Cd44, and not Cdh5, or Acta2;
f. one or more of Sox-9, Scleraxis (Scx), Spp1, Cspg4, CD73 (Nt5e), and Cartilage Intermediate Layer Protein (Cilp); or
g. one or more of S1004a, Dcn, Sema3c, or Cxcl12.

88. A method of preparing a bone marrow derived endothelial cell (BMEC) enriched cell population a stromal cell population comprising:
enriching the population of stromal cells for cells that have a BMEC gene signature, wherein the gene signature comprises
a. one or more genes of Table 6;
b. one or more of Mafb, Pparg, Nr2f2, Irf8, Ets1, Sox17, Sox11, Bcl6b, Gata2, Tcf15, Meox1, Sox7, Tshz2, Tfpi, Gpm6a, Ackr1, Mrc1, Stab1, Vcam1, Tek, Flt1, Ramp3, Icam2, Podx1, Cd34, Mcam, Sdpr, Bcam, Tspan13, Fabp5, Vim, Kit1, Lrg1, Dnasel13, Sepp1, Egfl7, Pde2a, Gpihbp1, Sema3g, Ramp2, Cd3001g, C1qtnf9, Sparcl1, Tinagl1, Pdgfb, Ubd, Stab2, Fabp4, Cldn5, Rgs4, Ecscr, Cyyr1, Ly6c1, Magix, Cav1, Gngt2, Myct1, or Tmsb4x;
c. one or more of Flt4 (Vegfr-3) or Ly6a (Sca-1);
d. one or more of Pecam1, Cdh5, Cd34, Tek, Lepr, Cxcl12, or Kitl;
e. one or more of Flt4, Ly6a, Icam1, or Sele;
f. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, lfitm2; or
g. one or more of Mafb, Cebpb, Xbp1, Nr2f2, Irf8, Ybx1, Ebf1, Sox17, Mxd4, Id1, Meox2, Tshz2, Tcf15, Meox1, Tfpi, Il6stm Angpt4, Gpm6a, Vcam1, Emp1, Cd34, Gnas, Slc9a3r2, Cald1, Mcam, Tspan13, Vim, Cd9, Ptrf, Crip2, Sepp1, Ctsl, Adamts5, Apoe, Igfbp4, Sparc, Col4a2, Col4a1, Serpinh1, Ppic, Cxcl12, Cst3, Sparcl1, C1qtnf9, Tinagl1, Mgll, Kit1, Stab2, Ubd, Gm1673, Abcc9, Rgs4, Ly6c1, Actg1, Tsc22d1, Glu1, Fxyd5, Crip1, Cav1, S100a6, S100a10, or lfitm2.

89. A method of preparing a pericyte enriched cell population a stromal cell population comprising:
enriching the population of stromal cells for cells that have a pericyte gene signature, wherein the gene signature comprises
a. one or more genes in Table 3;
b. one or more of Hey1, Nr2f2, Tbx2, Ebf1, Ebf2, Foxsl, Id3, Met2c, Cebpb, Zfxh3, Nr4a1, Klf9, Zeb2, Prrx1, Meox2, Junb, Id4, Zfp467, Irf1, Arid5b, Atp1b2, Aoc3, Sncq, Itga7, Aspn, Steap4, Thy1, Filip1I, Parm1, Agtr1a, Olfml2a, Cald1, Ednra, Col18a1, Serpini1, Bcam, Rrad, Pdgfrb, Col5a3, Pde5a, Notch3, Myl1, Tinagl1, Art3, Ngf, Sparcl1, 116, Rarres2, Vstm4, Pgf, Pdgfa, Col4a2, Igfbp7, Col4a1, Fst, Rtn4lrl1, Adamts1, 1134, Gpc6, Cscll, Bgs5, Tagln, Higd1p, Nrip2, Gucv1a3, H2-M9, Des, Olfr558, Lmod1, Gucy1b3, Kcnk3, Pdlim3, Gm13861, Mrvi1, Pln, Gm13889, Ral11a, or Cygp;
c. one or more of Cspg4, Ngfr, Des, Myh11, Acta2, Rgs5, Thy1, Pdgtfrb, Nes, Lepr, Cdh2, Cxcl12, Kitl. Ebf1, Sox4, Dlx5, Mxd4, Smad6, Hey1, Tcf15, Klf2, Mef2c, Atf3, Meox2, Steap4, Olfml2a, H2-M9, Tspan15, Cd24a, Marcks, Fbn1, Tnfrsf21, Slc12a2, Cfh, Cdh2, Vcam1, Sncg, Rasd1, Bcam, Rrad, Prkcdbp, Susd5, Csrrp1, Ptrf, Lama5, Ppp1r12b, Fhl1, Vim, Sdpr, Vtn, Angpt12, Cd44, Htra1, Mfap5, Anxa2, Procr, Igf1, Mgp, Col5a3, col4a2, Vstm4, Col3a1, Col4a1, Emcn, Gas1, Col6a2, Kit1, Sparcl1, Igfbp5, Ntf3, Inhba, Ccdc3, Fst, Timp3, Col1a1, Nbl1, Nov, Ccl11, Lga1s1, Dpt, Ctsl, Col6a3, Cxcl12, Rgs5, Abcc9, Phlda1, Tgs2, Cygb, Marcksl1, Apbb2, Ifitm3, Tmsb4x, Fam162a, Tagln, Pcp411, Crip1, Myl6, Acta2, Pln, Nrip2, Mustn1, Dstn, Mul9, Myh11, S100a6, Tppp3, Enpp2, S100a10, Cav1, Gstm1, Lysmd2, Myl12a, Nnmt, or S100a11; or
d. one or more of Acta2, Myh11, Mcam, Jag1, or Il6.

90. The method of any one of claims 84-89, wherein enriching the population of stromal cells comprises determining an MSC, an OLC, a chondrocyte, a BMEC, a fibroblast, a pericyte gene signature, or a combination thereof, wherein the gene signature(s) are determined by single cell RNA sequencing.

91. A method of detecting a hematological disease comprising:
a. determining a fraction of:
i. OLC-1 cells,
ii. OLC-2 cells,
iii. bone marrow derived endothelial cells (BMECs);
iv. chondrocytes;
v. fibroblasts; and
b. diagnosing the neurodegenerative disease in the subject when
i. the relative proportion of OLC-1 cells to OLC-2 cells is changed as compared to a suitable control;
ii. the fraction of OLC-1 cells is increased as compared to a suitable control;
iii. the fraction of OLC-2 cells is decreased as compared to a suitable control;
iv. the relative proportion of bone marrow derived endothelial fractions is changed as compared to a suitable control;
v. a fraction of sinusoidal BMECs is decreased as compared to a suitable control;
vi. a fraction of arterial BMECs is increased as compared to a suitable control;
vii. the relative proportion of chondrocyte fractions is changed as compared to a suitable control;
viii. a chondrocyte hypertorphic cell subtype is increased as compared to a suitable control;
ix. a chondrocyte progenitor cell subtype is decreased as compared to a suitable control;
x. a fibroblast subtype is changed as compared to a suitable control;
xi. a fibroblast subtype-3 is decreased; as compared to a suitable control
xii. a fibroblast subtype-4 is increased as compared to a suitable control;
xiii. the relative proportion of MSC fractions is changed as compared to a suitable control;
ixx. a MSC-2 fraction is increased as compared to a suitable control;
xx. a MSC-3 fraction is decreased as compared to a suitable control;
xxi. a MSC-4 fraction is decreased as compared to a suitable control; or
xxii. a combination thereof.

92. The method of claim 91, wherein the hematological disease is a blood cancer.

93. The method of claim 92, wherein the blood cancer is a leukemia.

94. The method of claim 93, wherein the blood cancer is acute lymphocytic leukemia, acute myeloid leukemia, chronic lymphocytic leukemia, chronic myeloid leukemia, hairy cell leukemia, myelodysplastic syndromes, acute promyelocytic leukemia, or myeloproliferative neoplasm.

95. A method of treating a hematological disease in a subject in need thereof, comprising:
detecting a hematological disease as in a subject according a method as in any one of claims 91-94; and
administering an effective amount of a hematological disease treatment to the subject.

96. The method of claim 95, wherein the hematological disease treatment comprises an agent selected from the group consisting of: cladribine, brentuximab vedotin, polatuzumab vedotin-piiq, fludarabine, fludarabine phosphate, mitoxantorone, etoposide, 6-thioguanine, hydroxyurea, methotrexate, 6-mercaptopurine, azacytidine, decitabine, daunorubicin, cyclophosphamide, daurismo, dexamethasome, cytarabine, arsenic trioxide, nelarabine, asparginase Erwinia chrysanthemi, calaspargase Pegol-mknl, inotuzumab ozogamicin, blinatumomab, clofarbine, dasatinib, dexamethasone, doxorubicin, imatinib mesylate, ponatinib, tisagenlecleucel, vincristine sulfate liposome, vincristine sulfate, mercaptopurine, methotrexate, pegaspargase, prednisone, hyper-CVAD, glasdegib maleate, enasidenib mesylate, gemtuzumab ozogamicin, gilteritinib fumarate, idarubicin, ivosidenib midostaurin, mitoxantrone, thioguanine, venetoclax, gilteritinib fumarate, tagraxofusp-erzs, acalabrutinib, alemtuzumab, ofatumumab, bendamustine HCl, chlorambucil, duvelisib, ibrutinib, idelalisib, mechlorethamine HCl, obinutuzumab, rituximab, hyaluronidase, idelalisib, bosutinib, hydroxyurea, busulfan, nilotinib, omacetaxine mepesuccinate, interferon alpha-2b, moxetumomab pasudotox-tdfk, bortezomib, romidepsin, belinostat, an immune checkpoint inhibitor (e.g. PD-1 inhibitors (e.g. pembrolizumab, nivolumab, and cemiplimab), PD-L1 inhibitors (e.g. atezolizumab, avelumab, and durvalumab), CTLA-4 targeting agents (e.g. ipilimumab), an immunomodulating agent (e.g. thalidomide and lenalidomide), a chimeric antigen receptor (CAR)-T cell therapy (e.g. axicabtagene ciloleucel and tisagenlecleucel), carboplatin, oxaliplatin, pentostatin, gemcitabine, pralatrexate, bleomycin, campath, acalabrutinib, zanubrutinib, idelalisib, copanlisib, duvelisib, and combinations thereof.

97. The method of claim 96, wherein the hematological disease treatment further comprises a stromal cell or cell population of any one of clusters 1-17 or a subtype thereof.

98. The method of claim 95, wherein the hematological disease treatment further comprises a stromal cell or cell population of any one of clusters 1-17 or a subtype thereof.


SEQUENCE LISTING (31 sequences)

SEQ ID NO: 1 - DNA, 37 bp, Artificial Sequence
  Synthetic
gcctgtccgcggaagcagtggtatcaacgcagagtac

SEQ ID NO: 2 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 3 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 4 - protein, 15 aa, Artificial Sequence
  Synthetic
GGGGGGGGGGGGGGG

SEQ ID NO: 5 - protein, 15 aa, Artificial Sequence
  Synthetic
GGGGSGGGGSGGGGS

SEQ ID NO: 6 - DNA, 10 bp, Artificial Sequence
  Synthetic
  misc_feature (5)..(5): n is a, c, g, or t
kccgnswttt

SEQ ID NO: 7 - DNA, 10 bp, Artificial Sequence
  Synthetic
yycattcaww

SEQ ID NO: 8 - DNA, 13 bp, Artificial Sequence
  Synthetic
  misc_feature (4)..(4): n is a, c, g, or t
  misc_feature (7)..(10): n is a, c, g, or t
ttcnrgnnnnttc

SEQ ID NO: 9 - DNA, 16 bp, Artificial Sequence
  Synthetic
  misc_feature (2)..(2): n is a, c, g, or t
  misc_feature (6)..(7): n is a, c, g, or t
  misc_feature (9)..(10): n is a, c, g, or t
  misc_feature (12)..(12): n is a, c, g, or t
rntcannrnnynattw

SEQ ID NO: 10 - DNA, 12 bp, Artificial Sequence
  Synthetic
  misc_feature (4)..(4): n is a, c, g, or t
  misc_feature (6)..(6): n is a, c, g, or t
ggcnrnwcttys

SEQ ID NO: 11 - DNA, 10 bp, Artificial Sequence
  Synthetic
  misc_feature (4)..(5): n is a, c, g, or t
gggnntttcc

SEQ ID NO: 12 - DNA, 10 bp, Artificial Sequence
  Synthetic
yycattcaww

SEQ ID NO: 13 - DNA, 14 bp, Artificial Sequence
  Synthetic
  misc_feature (4)..(9): n is a, c, g, or t
tggnnnnnnkccar

SEQ ID NO: 14 - DNA, 12 bp, Artificial Sequence
  Synthetic
  misc_feature (5)..(5): n is a, c, g, or t
  misc_feature (7)..(7): n is a, c, g, or t
acawnrnsrcgg

SEQ ID NO: 15 - DNA, 10 bp, Artificial Sequence
  Synthetic
  misc_feature (5)..(5): n is a, c, g, or t
atcmntccgy

SEQ ID NO: 16 - DNA, 14 bp, Artificial Sequence
  Synthetic
  misc_feature (6)..(7): n is a, c, g, or t
  misc_feature (9)..(11): n is a, c, g, or t
ccaatnnsnnngcg

SEQ ID NO: 17 - DNA, 13 bp, Artificial Sequence
  Synthetic
  misc_feature (5)..(5): n is a, c, g, or t
  misc_feature (8)..(10): n is a, c, g, or t
ccawnwwnnnggc

SEQ ID NO: 18 - DNA, 10 bp, Artificial Sequence
  Synthetic
  misc_feature (6)..(6): n is a, c, g, or t
ccawwnaagg

SEQ ID NO: 19 - DNA, 11 bp, Artificial Sequence
  Synthetic
  misc_feature (6)..(7): n is a, c, g, or t
ccawynngaar

SEQ ID NO: 20 - DNA, 11 bp, Artificial Sequence
  Synthetic
  misc_feature (4)..(5): n is a, c, g, or t
  misc_feature (7)..(7): n is a, c, g, or t
gcgnnanttcc

SEQ ID NO: 21 - DNA, 11 bp, Artificial Sequence
  Synthetic
  misc_feature (4)..(4): n is a, c, g, or t
  misc_feature (10)..(10): n is a, c, g, or t
ggcnkccatnk

SEQ ID NO: 22 - DNA, 10 bp, Artificial Sequence
  Synthetic
  misc_feature (4)..(4): n is a, c, g, or t
gtcnyyatgr

SEQ ID NO: 23 - DNA, 12 bp, Artificial Sequence
  Synthetic
  misc_feature (5)..(5): n is a, c, g, or t
  misc_feature (7)..(8): n is a, c, g, or t
raagnynnctty

SEQ ID NO: 24 - DNA, 10 bp, Artificial Sequence
  Synthetic
  misc_feature (6)..(6): n is a, c, g, or t
rcgcangcgy

SEQ ID NO: 25 - DNA, 13 bp, Artificial Sequence
  Synthetic
  misc_feature (7)..(9): n is a, c, g, or t
rytaawnnntgay

SEQ ID NO: 26 - DNA, 10 bp, Artificial Sequence
  Synthetic
  misc_feature (7)..(7): n is a, c, g, or t
stttcrnttt

SEQ ID NO: 27 - DNA, 11 bp, Artificial Sequence
  Synthetic
  misc_feature (5)..(5): n is a, c, g, or t
  misc_feature (7)..(8): n is a, c, g, or t
taaynrnntcc

SEQ ID NO: 28 - DNA, 12 bp, Artificial Sequence
  Synthetic
  misc_feature (4)..(5): n is a, c, g, or t
  misc_feature (7)..(7): n is a, c, g, or t
tttnnanagcyr

SEQ ID NO: 29 - DNA, 10 bp, Artificial Sequence
  Synthetic
  misc_feature (5)..(5): n is a, c, g, or t
wctcnatggy

SEQ ID NO: 30 - DNA, 12 bp, Artificial Sequence
  Synthetic
  misc_feature (7)..(8): n is a, c, g, or t
ywattwnnrgct

SEQ ID NO: 31 - DNA, 10 bp, Artificial Sequence
  Synthetic
yycattcaww